(19) United States
(12) Patent Application Publication
(10) Pub. No.: US 2026/0000642 A1
(43) Pub. Date: Jan. 1, 2026

(54) PYRROLES AND IMIDAZOLES AS BET PROTEIN INHIBITORS

(71) Applicant: Tay Therapeutics Limited, Dundee (GB)

(72) Inventors: Mark BELL, Dundee (GB); David William FOLEY, Dundee (GB); Christopher Andrew WOODLAND, Dundee (GB)

(21) Appl. No.: 19/023,992

(22) PCT Filed: Jul. 20, 2023
(86) PCT No.: PCT/IB2023/057419

§ 371 Date: Jan. 16, 2025

(30) Foreign Application Priority Data
Jul. 21, 2022 (GB) 2210714.8
Feb. 27, 2023 (GB) 2302871.5

(51) Int. Cl. A61K 31/401; A61K 31/4025; A61K 31/4439; A61P 13/02; A61P 19/02; C07D 207/36; C07D 401/12; C07D 403/04; C07D 405/12

(52) U.S. Cl. CPC A61K 31/401; A61K 31/4025; A61K 31/4439; A61P 13/02; A61P 19/02; C07D 207/36; C07D 401/12; C07D 403/04; C07D 405/12

(57) ABSTRACT

The disclosure relates to compounds of formula (I)

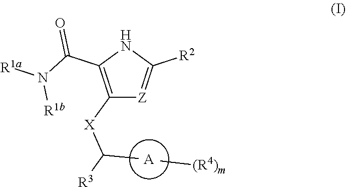

comprising a pyrrole or imidazole core, and pharmaceutically acceptable salts and compositions of such compounds. The compounds disclosed are useful as anti-inflammatory and/or other therapies.

### CROSS-REFERENCE TO RELATED APPLICATIONS

This application claims priority to United Kingdom Patent Application No. GB2210714.8, filed Jul. 21, 2022 and to United Kingdom Patent Application No. GB2302871.5, filed Feb. 27, 2023. The content of these applications are each incorporated herein by reference in their entirety.

This disclosure relates to compounds comprising a pyrrole or imidazole core, and pharmaceutically acceptable salts and compositions of such compounds. The compounds provided herein are useful as anti-inflammatory and/or other therapies. Therefore, the disclosure provides compounds for use as medicaments, for the treatment of diseases involving inflammation and or other disorders.

### BACKGROUND

Diseases and disorders may be multifactorial. They can involve inflammation or can result in inflammation related disorders. Autoimmune diseases and disorders may result in inflammation or may result in inflammation related disorders. An inflammatory or autoimmune disease or disorder may cause or result in changes, damage and/or wounds. A significant aspect of treatment of many diseases or disorders is to facilitate correct healing. A failed or failing healing process, a poor healing process, or an exaggerated healing process may, for example, leave lesions, erosion, wounds and/or fibrosis, and/or other damage.

The present disclosure is directed to methods for the treatment of arthritic diseases and disorders (e.g., joint related diseases and disorders) using potent and selective Bromodomain and Extra-Terminal (BET) inhibitors, and pharmaceutically acceptable salts thereof, their use for the treatment of diseases, and compositions/formulations comprising the BET inhibitors.

Joint or joint related disorders or diseases are diseases that affect human joints. Arthritis is one example of a well-known joint disease. Osteoarthritis is the most common form of arthritis and involves the wearing away of the cartilage that caps the bones in a person's joints. It is a degenerative joint disease characterized by joint pain and a progressive loss of articular cartilage. Although its etiology is still unknown, it is now acknowledged that during the inflammatory process of arthritis there are three key mediators, the proinflammatory cytokines TNF-α, IL-1β and IL-6 (see Mori T. et al., IL-1β and TNFα-initiated IL-6-STAT3 pathway is critical in mediating inflammatory cytokines and RANKL expression in inflammatory arthritis, International Immunology, Volume 23, Issue 11, 2011, pp. 701-712).

Regardless of the cause, inflammation of the joints may cause pain, stiffness, swelling, and some redness of the skin about the joint. Steroids (i.e., corticosteroids) are synthetic drugs that are used to treat a variety of inflammatory diseases and conditions. But the administration of corticosteroids, particularly for extended periods of time, can have a number of unwanted side effects or adverse reactions. The effectiveness of corticosteroids generally diminishes with time and there are disadvantages in their use, including a greater susceptibility to infection and peptic ulcers and corticosteroid injection directly into joint tissues may in some subjects worsen joint damage. For example, the unwanted adverse reactions of triamcinolone (which is a corticosteroid) injections include, hypersensitivity reactions, such as anaphylaxis, joint infection and damage, increased risk of infections, alterations in endocrine function, cardiovascular and renal effects, increased intraocular pressure, gastrointestinal perforation, alternations in bone density and behavioral and mood disturbances. As yet another example, the unwanted adverse reactions of dexamethasone (another corticosteroid) include, fluid and electrolyte disturbances, musculoskeletal, gastrointestinal, neurologic, dermatologic, endocrine, ophthalmic, metabolic cardiovascular, anaphylactoid or hypersensitivity reactions, thromboembolism, weight gain, increased appetite, and nausea (see, e.g., Brinks, A. et al. “Adverse effects of extra-articular corticosteroid injections: a systematic review.” BMC musculoskeletal disorders, 11:206, 2010).

Many disorders involve inflammation and share similar biomarker patterns and a product which is capable of reducing inflammatory cytokines involved in inflammation and treats or ameliorates the disorder while avoiding or minimizing side effects or adverse reactions would be advantageous and could improve patient compliance with treatment.

In addition to inflammation, fibrosis can be a problem throughout the body including in organs, such as the kidney, lungs, liver, heart, lymph nodes (e.g., mediastinal fibrosis), bone marrow, skin, tendons, joints, connective tissue, soft tissues, and cavities e.g., retroperitoneal. Fibrosis may be local or systemic.

Defined by the pathological accumulation of extracellular matrix (ECM) proteins, fibrosis results in scarring which may be coupled with thickening of the affected tissue—it is in essence an exaggerated wound healing response which interferes with normal organ function. Fibrosis of the lung is generally characterized by alveolar epithelial cell injury, areas of type II cell hyperplasia, accumulation of fibroblasts and myofibroblasts, and the deposition of extracellular matrix proteins. The result is a progressive loss of normal lung architecture and impairment in gas exchange. Accordingly, symptoms can include shortness of breath, a dry cough, feeling tired, weight loss, and nail clubbing (e.g., due to low oxygen in the blood). Fibrosis of the kidney is generally characterized by tubulointerstitial fibrosis, i.e., the deposition of connective tissue in the kidney parenchyma, and glomerulosclerosis, i.e., scarring of the glomerulus. The result is the progressive formation of internal scar tissue that leads to end-stage kidney failure. Accordingly, symptoms can include weight loss and poor appetite, edema (i.e., water retention), shortness of breath, fatigue, frequent urination, hematuria, and itchy skin.

Bromodomain and Extra-Terminal (BET) proteins are a family of four bromodomain-containing (BRD) proteins (BRD2, BRD3, BRD4 and BRDT). Bromodomain and Extra-Terminal (BET) proteins are a family of four bromodomain-containing (BRD) proteins (BRD2, BRD3, BRD4 and BRDT). All four members contain two BRDs (located next to each other toward the N-terminal of the proteins) and an extra-terminal domain (Shi, J. et al. Cancer Cell 25(2):210-225 (2014)). The two BRDs in each BET protein are designated bromodomain I (BDI) and bromodomain II (BDII). The BRD is a functional protein domain that contains a defined and predominantly hydrophobic pocket that binds to acetylated lysine residues, typically those found on transcription factors (Shi, J. et al. Cancer Cell 25(2):210-225 (2014)) or on the N-terminal tails of histone proteins. BRDs function as epigenetic regulators, i.e., they functionally alter gene activity and expression without altering the DNA sequence. For example, BRD4 recruits the transcription factor P-TEFb to promoters leading to altered expression of genes involved in the cell cycle (Yang et al., Mol. Cell Biol. 28: 967-976 (2008)). BRD2 and BRD3 also regulate growth promoting genes (LeRoy et al., Mol Cell 30:51-60 (2008)). Therefore, BRDs are responsible for transducing the signals carried by acetylated lysine residues into various phenotypes. BETs are considered in the art to be ubiquitously expressed in humans except for BRDT, which is normally expressed in the testes but is also expressed by some cancers (Ekaterina B. F. et al. Cell J. 19 (Suppl 1): 1-8 (2017)).

BET proteins have roles in the regulation of biochemical pathways such as MYC, BCL2, FOSL1, P-TEFb, NFkB, Glucocorticoid signalling and others (Shi J. et al. Mol Cell. June 5; 54(5):728-36 (2014)), (Hajmirza A. Biomedicines. February 6; 6(1). pii: E16 (2018)), (Shan N. Elife. September 11; 6. pii: e27861. (2017)), (Huang B. Mol Cell Biol. March; 29(5):1375-87 (2009)). As such, BET inhibitors are considered to have potential uses in a range of inflammatory diseases, cancers, infections, metabolic diseases, CNS disorders, fibrotic diseases, and cardiac diseases (Deanna A. M. et al. J Exp Med. October 21; 210(11): 2181-2190 (2013)), (Rab K. P. et al. Trends Pharmacol. Sci. March; 33(3):146-53 (2012)), (Anna C. B. et al. J Immunol. April 1; 190(7): 3670-3678 (2013)), (Zuber J. et al. Nature. August 3; 478(7370):524-8. (2011)), (Montserrat P. S. et al. Epigenetics; 12(5): 323-339 (2017)), (Qiming D. et al. Sci Transl Med. May 17; 9(390): eaah5084. (2017)), (Kristin M. K et al. J Biol Chem. August 11; 292 (32): 13284-13295 (2017)), (Ning D. et al. PNAS December 22, 112 (51) 15713-15718 (2015)).

The inhibition of BDII domain of BET proteins has been shown to effect inflammatory diseases, metabolic disease, cancers, and fibrotic diseases (Gilan et. al., Science 368, 387-394 (2020)), (L. M Tsujikawa et. al. Clin Epigenetics. 2019; 11(1):102), (E. Faivre et al. Nature 578, 306-310 (2020)), (M. Zhang, et al., Cellular Signalling 61 (2019) 20-29).

Compounds that can inhibit or affect the function of BET proteins have the potential to modulate gene expression and treat diseases that are at least in part caused by abnormal regulation of BET protein activity. Several small molecules have been reported to be effective in BET inhibition, including diazepine-, 3,5-dimethylisoxazole-, thiazol-2-one-, diazobenzene-, and 4-acylpyrrole-based compounds (see M. Brand et al, ACS Chem. Biol. 2015, 10, 22-39, WO2011054553, WO2011054845). Compounds that can selectively inhibit the function of BDII over BDI have the potential to modulate gene expression and treat diseases that are at least in part caused by abnormal regulation of BET protein activity, while offering the potential of an improved therapeutic index. Several small molecules have been reported to be effective in selectively inhibiting the function of BET BDII over BDI, including (BY27, RVX-297, ABBV744, GSK046, GSK620, GSK549 (Chen D. et. al. Eur J Med Chem 182, 2019, 111633), (Wells P. S. et. al. Proc. Natl. Acad. Sci. U.S.A 2013, 110, 19754-19759) (Sheppard G. S. et. al. J. Med. Chem. 2020, 63, 10, 5585-5623), (Preston A. et. al. J. Med. Chem. 2020, 63, 17, 9070-9092), (Seal J. T. et. al. J. Med. Chem. 2020, 63, 17, 9093-9126). Improved therapeutic index and pre-clinical safety of BDII selective BET inhibitors verses pan-BET inhibitors have been demonstrated (E. Faivre et al. Nature 578, 306-310 (2020)).

WO2018158212A1 discloses pyrazole derivatives which are bromodomain inhibitors. WO2020043821A1 discloses furan derivatives which are bromodomain inhibitors.

A product that is safe, well-tolerated, and prevents occurrence and/or reduces the grade of severity or the incidences, for example, of a joint or joint related disorders or diseases and/or fibrotic or fibrotic related disorders or diseases, while avoiding unwanted side effects and adverse reactions would be advantageous and could improve patient compliance with treatment. Accordingly, there is a medical need to replace corticosteroids with safer and better drugs in order to reduce the systemic side effects associated with the administration of corticosteroids. In addition, there is a medical need to slow, arrest, reverse, or otherwise inhibit structural damage to tissues caused by inflammatory diseases, such as damage to articular tissues resulting from, for example, osteoarthritis or rheumatoid arthritis. Involvement of bursas, tendons, and tendon sheaths can be part of arthritic disease. Similarly, there is a medical need to slow, arrest, reverse, or otherwise inhibit fibrosis and the negative consequences thereof including reduced organ function and ultimately failure. Compounds that can inhibit or affect the function of BET proteins have the potential to modulate gene expression and treat diseases that are at least in part caused by abnormal regulation of BET protein activity.

The present disclosure provides novel BET protein inhibitors.

### BRIEF SUMMARY OF THE DISCLOSURE

In accordance with a first aspect, the present disclosure provides a compound of formula (I), a tautomer, a stereoisomer or a mixture of stereoisomers, a pharmaceutically acceptable salt, a hydrate, a deuterated derivative, or N-oxide thereof:

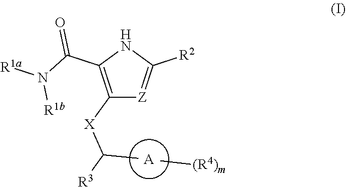

Ring A is independently selected from phenyl, 5-membered heterocyclyl, 6-membered heterocyclyl, 9-membered bicyclic heterocyclyl group, and 10-membered bicyclic heterocyclyl group;
X is independently selected from O and NR⁹;
Z is independently selected from N and CR¹⁰;
R^{1a} and R^{1b} are each independently selected from H, C₁-C₄-alkyl, C₂-C₄-alkenyl, C₂-C₄-alkynyl, C₁-C₄-haloalkyl, C₀-C₄-alkylene-R^{1c}, wherein R^{1c} is independently selected from C₃-C₆ cycloalkyl, 4-membered heterocyclyl, 5-membered heterocyclyl, and 6-membered heterocyclyl; wherein R^{1c} is optionally substituted with from 1 to 4 R^{1d};
or R^{1a} and R^{1b} together with the nitrogen atom to which they are attached form a 5- to 8-membered heterocycloalkyl group optionally substituted with from 1 to 4 R^{1e};
R² is independently selected from —CONR^{2a}R^{2b}, —NR^{2a}COR^{2g}, 5-membered heterocyclyl, 6-membered heterocyclyl, and phenyl, wherein the 5-membered heterocyclyl, and 6-membered heterocyclyl groups may be optionally substituted with from 1 to 4 R^{2c} and wherein the phenyl group may be optionally substituted with from 1 to 5 R^{2c};
wherein R^{2a} and R^{2b} are each independently selected from H, C₁-C₄-alkyl, C₂-C₄-alkenyl, C₂-C₄-alkynyl, C₁-C₄-haloalkyl, C₀-C₄-alkylene-R^{2d}; wherein R^{2d} is independently selected from C₃-C₆ cycloalkyl, 4-membered heterocyclyl, 5-membered heterocyclyl, and 6-membered heterocyclyl, wherein R^{2d} is optionally substituted with from 1 to 4 R^{2e};
or R^{2a} and R^{2b} together with the nitrogen atom to which they are attached form a 5- to 8-membered heterocycloalkyl group optionally substituted with from 1 to 4 R^{2f};
wherein R^{2g} is independently selected from C₁-C₄-alkyl, C₂-C₄-alkenyl, C₂-C₄-alkynyl, C₁-C₄-haloalkyl, C₀-C₄-alkylene-R^{2d}; wherein R^{2d} is independently selected from C₃-C₆cycloalkyl, 4-membered heterocyclyl, 5-membered heterocyclyl, and 6-membered heterocyclyl, wherein R^{2d} is optionally substituted with from 1 to 4 R^{2e};
or R^{2a} and R^{2g} together with the atoms to which they are attached form a 5- to 8-membered heterocycloalkyl group optionally substituted with from 1 to 4 R^{2f};
R^{1d}, R^{1e}, R^{2c}, R^{2e} and R^{2f} are each independently at each occurrence selected from ═O, ═S, halo, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₄-cycloalkyl, 4-membered heterocyclyl, 5-membered heterocyclyl, and 6-membered heterocyclalkyl;
R³ is independently selected from H, C₁-C₄-alkyl, C₂-C₄-alkenyl, C₂-C₄-alkynyl, C₁-C₄-haloalkyl, C₂-C₄-haloalkenyl, C₁-C₄-alkylene-OR⁷, C₀-C₄-alkylene-S(O)₂R⁶, C₀-C₄-alkylene-CONR⁶R⁶, C₃-C₄-cycloalkyl, 4-membered heterocyclyl, 5-membered heterocyclyl, and 6-membered heterocyclyl;
R⁴ is independently at each occurrence selected from ═O, ═S, halo, nitro, cyano, C₀-C₄-alkylene-NR⁵R⁶, C₀-C₄-alkylene-OR⁷, SR⁶, SOR⁶, C₀-C₄-alkylene-S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl, C₁-C₄-alkyl-S(O)₂R⁶, C₂-C₄-alkenyl, C₂-C₄-alkynyl, C₁-C₄-haloalkyl, C₃-C₄-cycloalkyl and 4-membered heterocycloalkyl;
R⁵ is independently at each occurrence selected from H, C₁-C₄-alkyl, C(O)—C₁-C₄-alkyl and S(O)₂—C₁-C₄-alkyl; and
R⁶ is independently at each occurrence selected from H and C₁-C₄-alkyl; or where two R⁶ groups are attached to the same nitrogen, those two R⁶ groups together with the nitrogen atom to which they are attached optionally form a 5- to 8-membered-heterocycloalkyl group optionally substituted with from 1 to 4 R⁸;
or R⁵ and R⁶ together with the nitrogen atom to which they are attached form a 5- to 8-membered heterocycloalkyl group optionally substituted with from 1 to 4 R⁸;
R⁷ is independently at each occurrence selected from H, C₁-C₄-alkyl, C(O)—C₁-C₄-alkyl and C₁-C₄-haloalkyl;
R⁸ is independently at each occurrence selected from ═O, ═S, fluoro, nitro, cyano, NR—R⁶, OR⁷, SR⁶, SOR⁶, S(O)₂R⁶, SO₂NR⁶R⁶, CO₂R, C(O)R⁶, CONR⁶R⁶, C₁-C₄-alkyl, C₂-C₄-alkenyl, C₂-C₄-alkynyl, C₁-C₄-haloalkyl, C₃-C₄-cycloalkyl and 4-membered heterocycloalkyl;
R⁹ is independently selected from H, C₁-C₄-alkyl, C₂-C₄-alkenyl, C₂-C₄-alkynyl, C₁-C₄-haloalkyl, C₂-C₄-haloalkenyl and C₃-C₄-cycloalkyl;
R¹⁰ is independently selected from H, halo, C₁-C₄-alkyl, C₂-C₄-alkenyl, C₂-C₄-alkynyl, C₁-C₄-haloalkyl, C₂-C₄-haloalkenyl, C₀-C₄-alkylene-OR⁷ and C₃-C₆-cycloalkyl; and
m is an integer selected from 0, 1, 2, 3 and 4;
wherein any of the aforementioned alkyl, alkylene, alkenyl, or cyclopropyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a}, and S(O)₂R^{a}; wherein R^{a} is independently at each occurrence selected from H and C₁-C₄-alkyl; and R^{b} is independently at each occurrence selected from H, C₁-C₄-alkyl, C(O)—C₁-C₄-alkyl and S(O)₂—C₁-C₄-alkyl.

The compounds of formula (I) may be an enantiomer, a mixture of enantiomers, a racemate, a diastereoisomer, a mixture of diastereoisomers, a geometric isomer, a mixture of geometric isomers, a tautomer or a mixture of tautomers. The compound of formula (I) may also be in the form of a solvate or hydrate. The compounds of formula (I) may also be in the form of a deuterated derivative.

In an embodiment, the compound of formula (I), or a pharmaceutically acceptable salt or N-oxide thereof, is a compound of formula (IIA) or (IIB):

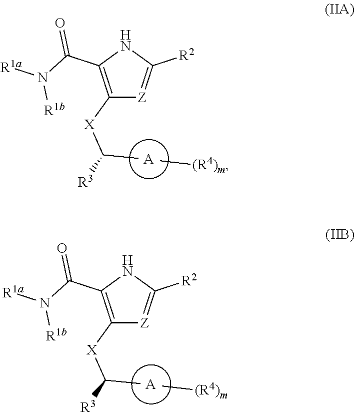

or a pharmaceutically acceptable salt or N-oxide thereof,
wherein Ring A, X, Z, R^{1a}, R², R³, R⁴ and m are as described above for compounds of formula (I).

In an embodiment, the compound of formula (I) is a compound of formula (IIIA) or (IIIB):

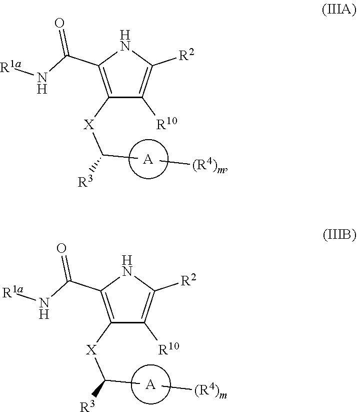

wherein Ring A, X, R^{1a}, R², R³, R⁴, R¹⁰ and m are as described above for compounds of formula (I).

In an embodiment, the compound of formula (I) is a compound of formula (IVA) or (IVB):

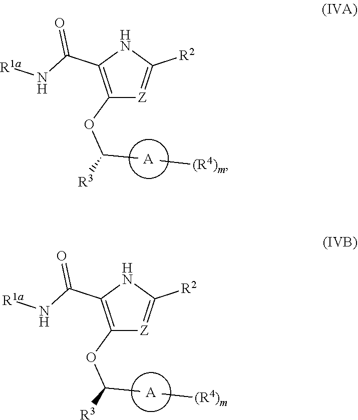

wherein Ring A, Z, R^{1a}, R², R³, R⁴ and m are as described above for compounds of formula (I).

In an embodiment, the compound of formula (I) is a compound of formula (VA) or (VB):

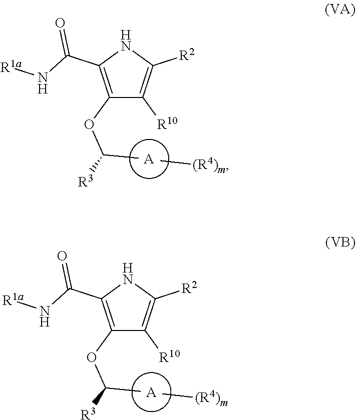

wherein Ring A, R^{1a}, R², R³, R⁴, R¹⁰ and m are as described above for compounds of formula (I).

In an embodiment, the compound of formula (I) is a compound of formula (VIA) or (VIB):

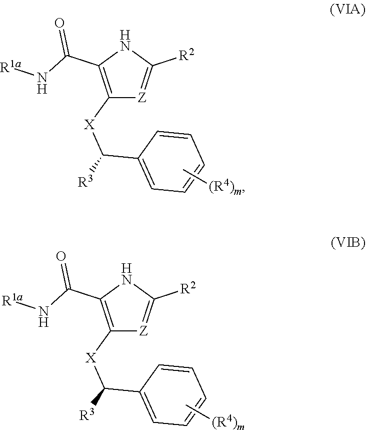

wherein X, Z, R^{1a}, R², R³, R⁴ and m are as described above for compounds of formula (I).

In an embodiment, the compound of formula (I) is a compound of formula (VIIA) or VIIB)

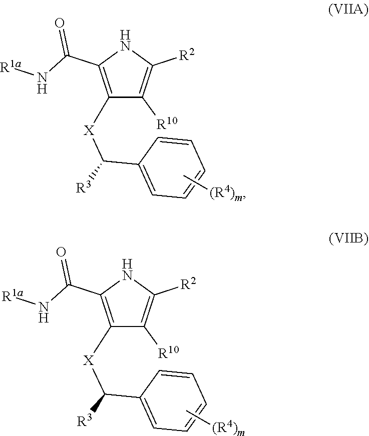

wherein X, R^{1a}, R², R³, R⁴, R¹⁰ and m are as described above for compounds of formula (I).

In an embodiment, the compound of formula (I) is a compound of formula (VIIIA) or (VIIIB):

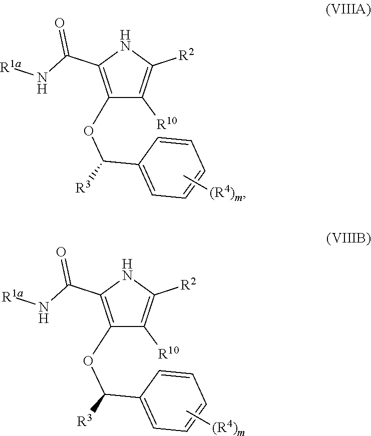

wherein R^{1a}, R², R³, R⁴, R¹⁰ and m are as described above for compounds of formula (I).

In an embodiment, the compound of formula (I) is a compound of formula (IXA) or (IXA):

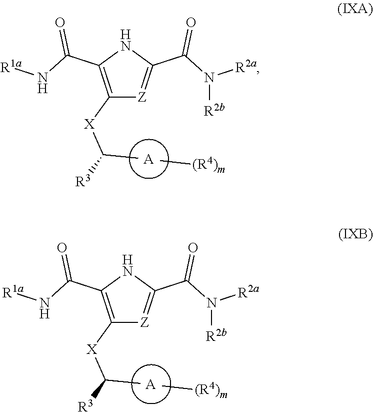

wherein Ring A, X, Z, R^{1a}, R^{2a}, R^{2b}, R³, R⁴ and m are as described above for compounds of formula (I).

In an embodiment, the compound of formula (I) is a compound of formula (XA) or (XB):

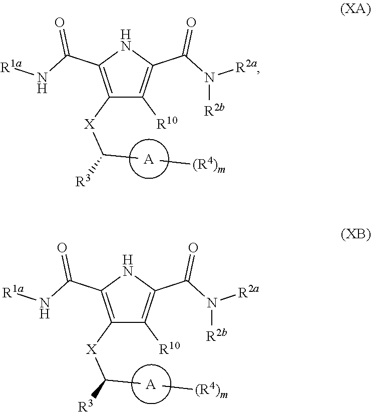

wherein Ring A, X, R^{1a}, R^{2a}, R^{2b}, R³, R⁴, R¹⁰ and m are as described above for compounds of formula (I).

In an embodiment, the compound of formula (I) is a compound of formula (XIA) or (XIB):

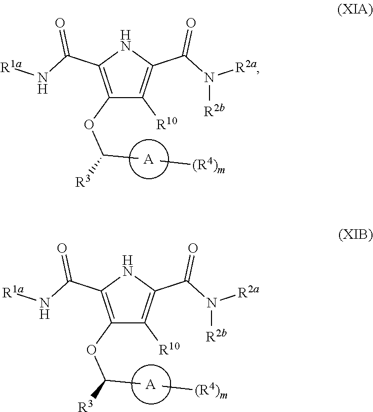

wherein Ring A, R^{1a}, R^{2a}, R^{2b}, R³, R⁴, R¹⁰ and m are as described above for compounds of formula (I).

In an embodiment, the compound of formula (I) is a compound of formula (XIIA) or (XIIB):

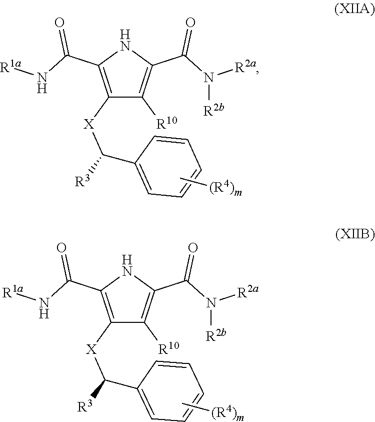

wherein X, R^{1a}, R^{2a}, R^{2b}, R³, R⁴, R¹⁰ and m are as described above for compounds of formula (I).

In an embodiment, the compound of formula (I) is a compound of formula (XIIIA) or (XIIIB):

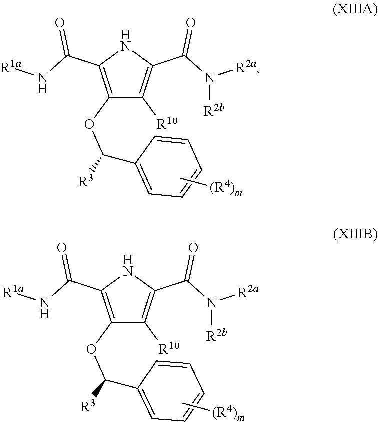

wherein R^{1a}, R^{2a}, R^{2b}, R³, R⁴, R¹⁰ and m are as described above for compounds of formula (I).

In an embodiment, the compound of formula (I) is a compound of formula (XIVA) or (XIVB):

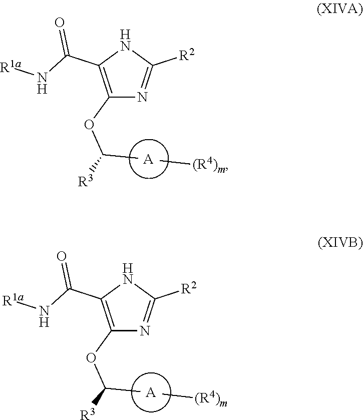

wherein Ring A, R^{1a}, R², R³, R⁴, and m are as described above for compounds of formula (I).

In an embodiment, the compound of formula (I) is a compound of formula (XVA) or (XVB):

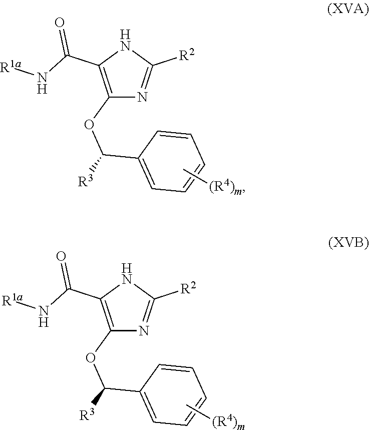

wherein R^{1a}, R², R³, R⁴, and m are as described above for compounds of formula (I).

In an embodiment, the compound of formula (I) is a compound of formula (XVIA) or (XVIB):

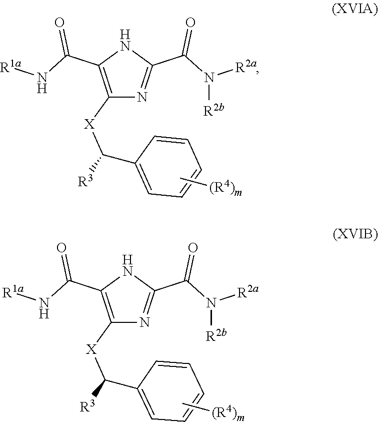

wherein X, R^{1a}, R^{2a}, R^{2b}, R³, R⁴, and m are as described above for compounds of formula (I).

In an embodiment, the compound of formula (I) is a compound of formula (XVIIA) or (XVIIB):

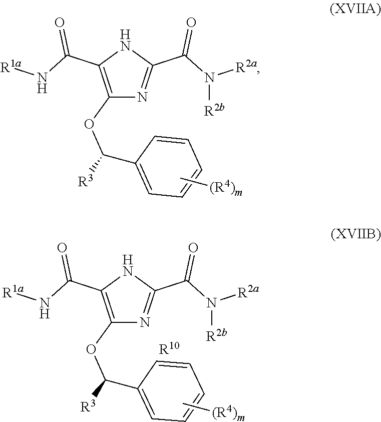

wherein R^{1a}, R^{2a}, R^{2b}, R³, R⁴, and m are as described above for compounds of formula (I).

The following embodiments apply to compounds of any of formulae (I)-(XVII), including both A and B configurations of formulae (II) to (XVII). These embodiments are independent and interchangeable. Any one embodiment may be combined with any other embodiment, where chemically allowed. In other words, any of the features described in the following embodiments may (where chemically allowable) be combined with the features described in one or more other embodiments. In particular, where a compound is exemplified or illustrated in this specification, any two or more of the embodiments listed below, expressed at any level of generality, which encompass that compound may be combined to provide a further embodiment which forms part of the present disclosure.

Where R³ is not H, the compound of formula (I) contains at least one chiral centre, i.e., the carbon to which R³ and Ring A are attached. Where the compound contains no further chiral centres, a molecule of formula (I) will be one of two enantiomers, varying in the configuration at this position. Where the compound of formula (I) contains one or more additional chiral centres, each molecule of formula (I) will be one of at least two diastereoisomers (varying in terms of the relative configurations of the two or more chiral centres) and one of two enantiomers (varying at the configuration at the carbon to which R³ and Ring A are attached. The compound of formula (I) may be in the form of a mixture of two enantiomers. The compound of formula (I) may be in the form of a racemic mixture of two enantiomers. The compound of formula (I) may be substantially in the form of a single enantiomer. The compound of formula (I) may be in the form of a single enantiomer. The compound of formula (I) may be in the form of a mixture of diastereoisomers. The compound of formula (I) may be substantially in the form of a single diastereoisomer. The compound of formula (I) may be in the form of a single diastereoisomer.

The compound of formula (I) may be in the form of a mixture of enantiomers or epimers that vary at the carbon to which R³ and Ring A are attached. Where the compound of formula (I) is a mixture of enantiomers that vary at this position, it may be in the form of a racemic mixture. The compound of formula (I) may be substantially in the form of a single enantiomer having the R configuration at the carbon to which R³ and Ring A are attached. The compound of formula (I) may be substantially in the form of a single enantiomer having the S configuration at the carbon to which R³ and Ring A are attached.

The compound of formula (I) may be substantially in the form of a single enantiomer having the configuration at the carbon to which R³ and Ring A are attached shown in formula (IIA). The compound of formula (I) may be substantially in the form of a single enantiomer having the configuration at the carbon to which R³ and Ring A are attached to that shown in formula (IIB). Where R³ has a lower priority assignment than Ring A under the Cahn-Ingold-Prelog rules, the compounds of formula (IIA) have the S configuration at the carbon to which R³ and Ring A are attached. Where R³ has a higher priority assignment than Ring A under the Cahn-Ingold-Prelog rules, the compounds of formula (IIA) have the R configuration at the carbon to which R³ and Ring A are attached.

The word ‘substantially’ may mean that the compound of formula (I) has an enantiomeric excess of about 90% or greater. The word ‘substantially’ typically means that the compound of formula (I) has an enantiomeric excess of about 95% or greater. It may mean that the compound of formula (I) has an enantiomeric excess of about 98% or greater, about 99% or greater or about 99.5% or greater.

In one or more embodiments certain pyrrole or imidazole compounds are provided in which the carbon to which R³ and Ring A are attached has the down configuration shown in e.g., formula (IIA). In one or more embodiments certain pyrrole or imidazole compounds are provided in which the carbon to which R³ and Ring A are attached has the up configuration shown in e.g., formula (IIB).

In one or more embodiments certain pyrrole or imidazole compounds are provided as S-enantiomers having the S configuration at the carbon to which R³ and Ring A are attached. In one or more embodiments certain pyrrole or imidazole compounds are provided as R-enantiomers having the R configuration at the carbon to which R³ and Ring A are attached.

In one or more embodiments certain pyrrole or imidazole compounds have a higher activity where the carbon to which R³ and Ring A are attached has the down absolute configuration (e.g., as depicted in formula (IIA)) than where the carbon to which R³ and Ring A are attached has the up absolute configuration (e.g., as depicted in formula (IIB)). In one or more embodiments certain pyrrole or imidazole compounds have a higher selectivity where the carbon to which R³ and Ring A are attached has the down absolute configuration (e.g., as depicted in formula (IIA)) than where the carbon to which R³ and Ring A are attached has the up absolute configuration (e.g., as depicted in formula (IIB)). In one or more embodiments certain pyrrole or imidazole compounds have a higher bioavailability where the carbon to which R³ and Ring A are attached has the down absolute configuration (e.g., as depicted in formula (IIA)) than where the carbon to which R³ and Ring A are attached has the up absolute configuration (e.g., as depicted in formula (IIB)).

The use of a compound as a drug is multifactorial and depends on many factors, including the disease or disorder, the mode of administration, activity, selectivity, pharmacokinetics, clearance, bioavailability, ability to modulate biomarkers effectively to ameliorate or treat a disease or disorder and the carrier selected to deliver the drug. In one or more embodiments certain pyrrole or imidazole compounds disclosed herein having a higher bioavailability and selectivity may be useful drugs, for example, where delivery is oral. In some other embodiments, certain other pyrrole or imidazole compounds having a lower bioavailability and or selectivity can be useful drugs, for example where delivery is by injection.

In one or more embodiments compounds in which the carbon to which R³ and Ring A are attached has the down configuration are useful drugs. In one or more other embodiments compounds in which the carbon to which R³ and Ring A are attached has the up configuration are useful drugs.

In one or more embodiments compounds in which the carbon to which R³ and Ring A are attached has the down configuration are useful tools e.g., diagnostic tools. In one or more other embodiments compounds in which the carbon to which R³ and Ring A are attached has the up configuration are useful tools e.g., diagnostic tools.

In an embodiment, Ring A is a 9-membered bicyclic heterocyclyl group. In an embodiment, Ring A is a 9-membered bicyclic heteroaryl group. In an embodiment, Ring A is a 5- or 6-membered heterocyclyl. In an embodiment, Ring A is a 5- or 6-membered heteroaryl. In an embodiment, Ring A is a 5-membered heteroaryl ring. In an embodiment, Ring A is a 6-membered heterocyclyl ring. In an embodiment, Ring A is a 6-membered heteroaryl ring. In an embodiment, Ring A is pyridyl. In an embodiment, Ring A is an indolyl group. In an embodiment, Ring A is a phenyl ring. Ring A may be unsubstituted, i.e., m may be 0. Ring A may be substituted with from 1 to 4 R⁴ groups, i.e., m may be from 1 to 4. In an embodiment, Ring A is not pyridazinyl. In an embodiment, Ring A is not a pyridazin-4-yl.

In an embodiment, where Ring A is pyridyl, Ring A is pyrid-3-yl, e.g.,

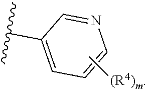

In an embodiment, where Ring A is pyridyl, Ring A is pyrid-4-yl, e.g.,

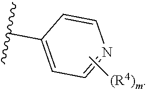

In an embodiment, Ring A is not pyrid-2-yl. In an embodiment, Ring A is not

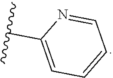 [SMILES: *c1ccccn1]

In an embodiment, where Ring A is phenyl, and n is 2, at least one R⁴ is not halo. In an embodiment, where Ring A is phenyl, and n is 2, at least one R⁴ is not F. In an embodiment, Ring A is not

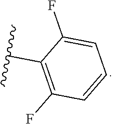 [SMILES: *c1c(F)cccc1F]

In an embodiment, Ring A is not

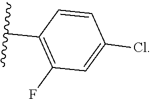 [SMILES: *c1ccc(Cl)cc1F]

In an embodiment, X is O. In an embodiment, X is NR⁹.

In an embodiment, Z is N. In an embodiment, Z is CR¹⁰.

In an embodiment, R^{1a} and R^{1b} are each independently selected from H, C₁-C₄-alkyl, C₁-C₄-haloalkyl and C₀-C₄-alkylene-R^{1c}. In an embodiment, R^{1a} is C₁-C₄-alkyl, e.g., methyl. In an embodiment, R^{1b} is H. In an embodiment, R^{1a} is methyl and R^{1b} is H. R^{1c} may be C₃-C₆ cycloalkyl, e.g., cyclopropyl.

In an embodiment, R^{1a} and R^{1b} together with the nitrogen atom to which they are attached form a 5- to 8-membered-heterocycloalkyl group optionally substituted with from 1 to 4 R^{1e} groups.

In an embodiment, R² is independently selected from —CONR^{2a}R^{2b}, 5-membered heterocyclyl, 6-membered heterocyclyl, and phenyl, wherein the 5-membered heterocyclyl, and 6-membered heterocyclyl groups may be optionally substituted with from 1 to 4 R^{2c} and wherein the phenyl group may be optionally substituted with from 1 to 5 R^{2c}.

In an embodiment, R² is —CONR^{2a}R^{2b}. In an embodiment, R² is a 5- or 6-membered heterocyclyl group e.g., imidazolyl, optionally substituted with from 1 to 4 R^{2c} groups. In an embodiment, R² is imidazolyl. In an embodiment, R² is phenyl, optionally substituted with from 1 to 4 R^{2c} groups. In an embodiment, R² is phenyl.

In an embodiment, R^{2a} and R^{2b} are each independently selected from H, C₁-C₄-alkyl, C₁-C₄-haloalkyl, C₀-C₄-alkylene-R^{2d}; wherein R^{2d} is independently selected from: C₃-C₆ cycloalkyl, and 4- to 6-membered heterocyclyl; wherein where R^{2d} is cycloalkyl or heterocycloalkyl, R^{2d} is optionally substituted with from 1 to 4 R^{2e} groups. In an embodiment, R^{2a} is C₁-C₄-alkyl, e.g., methyl or ethyl. In an embodiment, R^{2b} is H. Thus, in an embodiment, R^{2a} is ethyl and R^{2b} is H. In an embodiment, R^{2a} is C₃-C₆ cycloalkyl, e.g., cyclopropyl, cyclobutyl, bicyclobutyl, cyclopentyl or bicyclopentyl. In an embodiment, R^{2a} is a 4- to 6-membered heterocyclyl group e.g., oxetane or tetrahydrofuran. In an embodiment, R^{2a} is C₁-C₄-haloalkyl, e.g., trifluoropropyl.

In an embodiment, R^{2a} and R^{2b} together with the nitrogen atom to which they are attached form a 5- to 8-membered-heterocycloalkyl group optionally substituted with from 1 to 4 R^{2f} groups.

In an embodiment, R² is —NR^{2a}COR^{2g}.

In an embodiment, R³ is independently selected from H, C₁-C₄-alkyl, C₂-C₄-alkenyl, C₂-C₄-alkynyl, C₁-C₄-haloalkyl, C₂-C₄-haloalkenyl, C₁-C₄-alkylene-OR⁷, C₃-C₄-cycloalkyl. In an embodiment, R³ is independently selected from C₁-C₄-alkyl, C₂-C₄-alkenyl, C₂-C₄-alkynyl, C₁-C₄-haloalkyl, C₂-C₄-haloalkenyl, C₁-C₄-alkylene-OR⁷, C₀-C₄-alkylene-S(O)₂R⁶, C₀-C₄-alkylene-CONR⁶R⁶, C₃-C₄-cycloalkyl and 4- to 6-membered heterocyclyl. In an embodiment, R³ is independently selected from C₁-C₄-alkyl, C₂-C₄-alkenyl, C₂-C₄-alkynyl, C₁-C₄-haloalkyl, C₂-C₄-haloalkenyl, C₁-C₄-alkylene-OR⁷ and C₃-C₄-cycloalkyl. R³ may be H. In an embodiment, R³ is C₁-C₄-alkyl, e.g., methyl, ethyl, propyl, butyl. In an embodiment, R³ is methyl. In an embodiment, R³ is ethyl. In an embodiment, R³ is C₁-C₄-haloalkyl, e.g., trifluoromethyl. In an embodiment, R³ is C₁-C₄-alkylene-OR⁷, e.g., methoxymethyl.

In an embodiment, R⁴ is independently at each occurrence selected from C₁-C₄-alkyl, halo, cyano, C₁-C₄-haloalkyl, and C₁-C₄-alkylene-OR⁷. In an embodiment, R⁴ is C₁-C₄-alkyl e.g., methyl. In an embodiment, R⁴ is halo, e.g., F or C₁. In an embodiment, R⁴ is cyano. In an embodiment, R⁴ is C₁-C₄-haloalkyl, e.g., CF₃. In an embodiment, R⁴ is C₁-C₄-alkylene-OR⁷, e.g., OMe.

In an embodiment, m is an integer selected from 0, 1, and 2. In an embodiment, m is 2. In an embodiment, m is 1. In an embodiment, m is 0.

In an embodiment, R⁵ is independently at each occurrence selected from H, C₁-C₄-alkyl, and S(O)₂—C₁-C₄-alkyl.

In an embodiment, R⁵ is S(O)₂—C₁-C₄-alkyl; optionally wherein R⁵ is S(O)₂—C-alkyl. In an embodiment, R⁵ is H. In an embodiment, R⁵ is methyl.

In an embodiment, R⁶ is independently at each occurrence selected from H and C₁-C₄-alkyl. In an embodiment, R⁶ is H. In an embodiment, R⁶ is methyl.

In an embodiment, R⁷ is independently at each occurrence selected from H, C₁-C₄-alkyl, and C₁-C₄-haloalkyl.

In an embodiment, R⁷ is independently at each occurrence selected from H, and C₁-C₄-alkyl.

In an embodiment, R⁷ is independently at each occurrence selected from H, C₁-C₂-alkyl, and C₁-C₂-haloalkyl.

In an embodiment, R⁷ is independently at each occurrence selected from H, and C₁-C₂-alkyl.

In an embodiment, R⁷ is independently at each occurrence H.

In an embodiment, R⁸ is independently at each occurrence selected from ═O, fluoro, nitro, cyano, NR⁵R⁶, OR⁷, C(O)R⁶, C₁-C₄-alkyl, C₁-C₄-haloalkyl and cyclopropyl.

In an embodiment, R⁸ is independently at each occurrence selected from ═O, fluoro, C(O)R⁶, C₁-C₂-alkyl, and C₁-C₂-haloalkyl.

In an embodiment, R⁸ is independently at each occurrence selected from ═O, fluoro, and C(O)R⁶. In an embodiment, R⁸ is independently at each occurrence selected from ═O, fluoro, and C(O)Me.

In an embodiment, R⁹ is independently at each occurrence selected from H, C₁-C₄-alkyl, C₁-C₄-haloalkyl and cyclopropyl.

In an embodiment, R⁹ is independently at each occurrence selected from H, C₁-C₄-alkyl, and C₁-C₄-haloalkyl.

In an embodiment, R⁹ is independently at each occurrence selected from H, C₁-C₂-alkyl, and C₁-C₂-haloalkyl.

In an embodiment, R⁹ is independently at each occurrence selected from H, and C₁-C₂-alkyl. In an embodiment, R⁹ is H. In an embodiment, R⁹ is methyl.

In an embodiment, R¹⁰ is independently selected from H, halo, C₁-C₄-alkyl, C₂-C₄-alkenyl, C₂-C₄-alkynyl, C₁-C₄-haloalkyl, C₂-C₄-haloalkenyl, C₀-C₄-alkylene-OR⁷ and C₃-C₆-cycloalkyl.

In an embodiment, R¹⁰ is independently at each occurrence selected from H, halo, C₁-C₄-alkyl, C₁-C₄-haloalkyl and cyclopropyl.

In an embodiment, R¹⁰ is independently at each occurrence selected from H, C₁-C₄-alkyl, C₁-C₄-haloalkyl and cyclopropyl.

In an embodiment, R¹⁰ is independently at each occurrence selected from H, halo, C₁-C₄-alkyl, and C₁-C₄-haloalkyl.

In an embodiment, R¹⁰ is independently at each occurrence selected from H, C₁-C₄-alkyl, and C₁-C₄-haloalkyl.

In an embodiment, R¹⁰ is independently at each occurrence selected from H, halo, C₁-C₂-alkyl, and C₁-C₂-haloalkyl.

In an embodiment, R¹⁰ is independently at each occurrence selected from H, C₁-C₂-alkyl, and C₁-C₂-haloalkyl.

In an embodiment, R¹⁰ is independently at each occurrence selected from H, Br, and C₁-C₂-alkyl. In an embodiment, R¹⁰ is independently at each occurrence selected from H, and C₁-C₂-alkyl. In an embodiment, R¹⁰ is H. In an embodiment, R¹⁰ is methyl. In an embodiment, R¹⁰ is Br.

In an embodiment, any of the alkyl or alkenyl groups are optionally substituted, where chemically possible, by 1 to 5 substituents which are each independently at each occurrence selected from the group consisting of: oxo, fluoro, NR^{a}R^{b}, OR^{a}, and S(O)₂R^{a}; wherein R^{a} is independently at each occurrence selected from H, and C₁-C₄-alkyl; and R^{b} is independently at each occurrence selected from H, C₁-C₄-alkyl, C(O)—C₁-C₄-alkyl and S(O)₂—C₁-C₄-alkyl.

In an embodiment, the compound according to formula (I) is selected from:

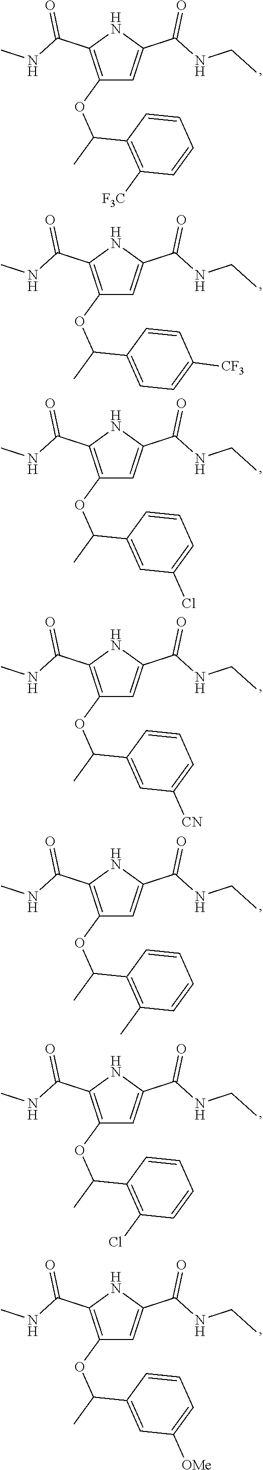

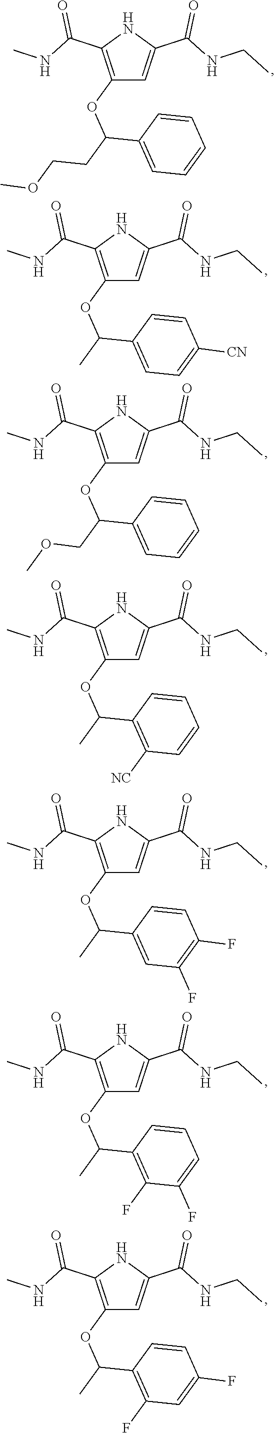

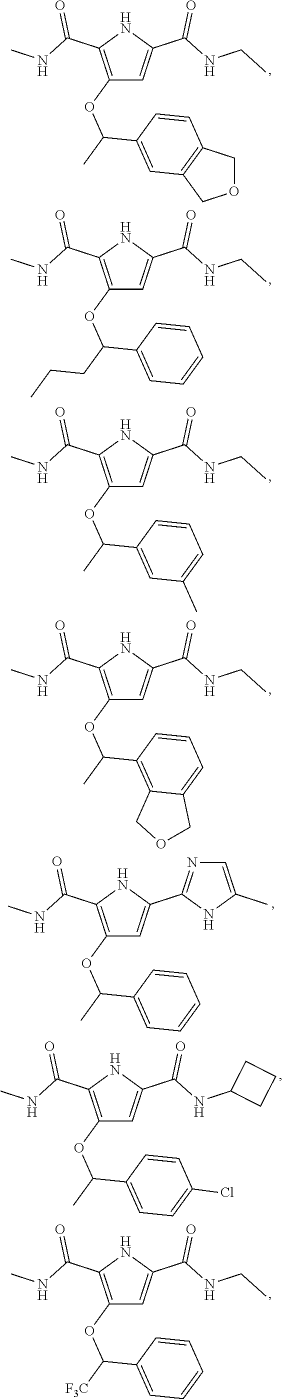

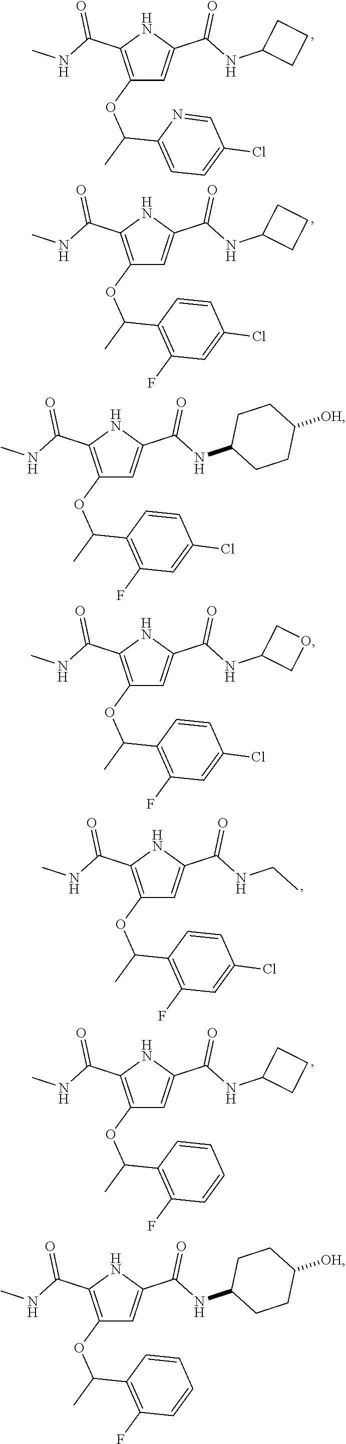

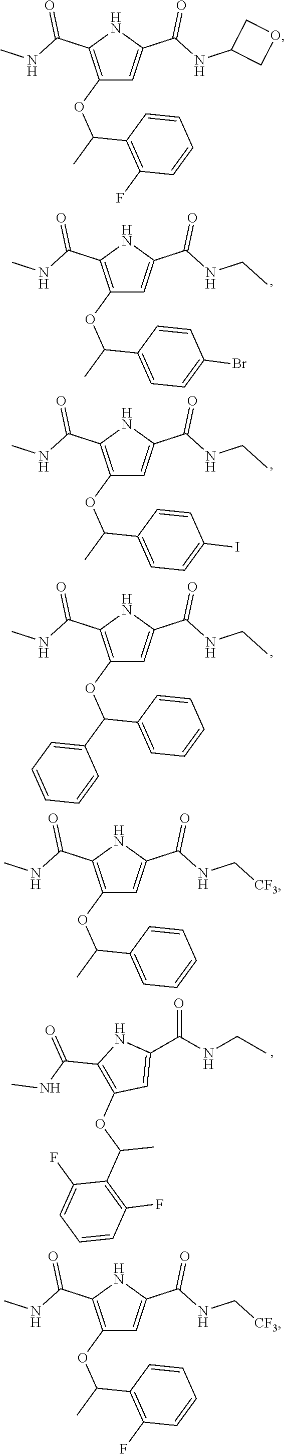

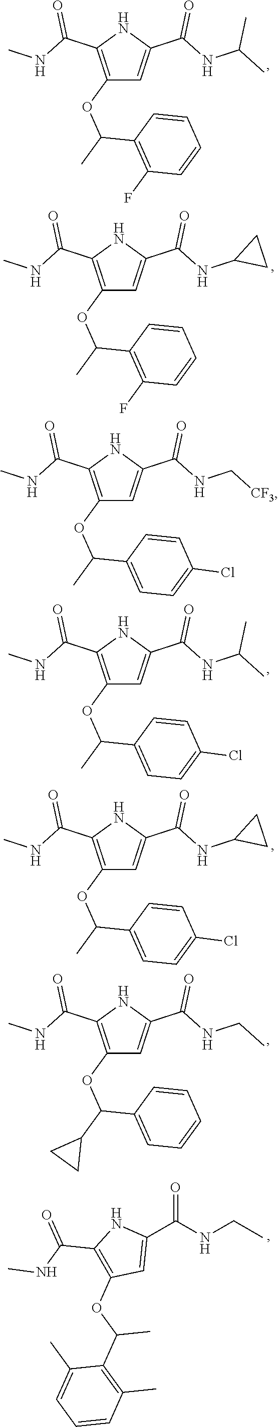

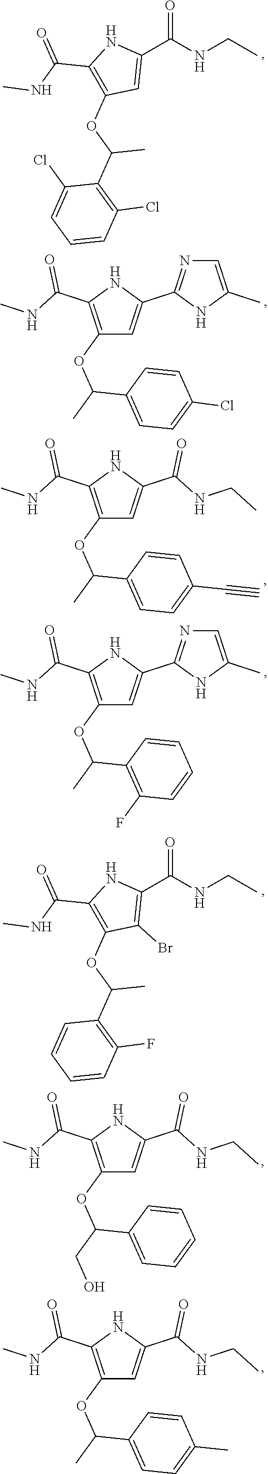

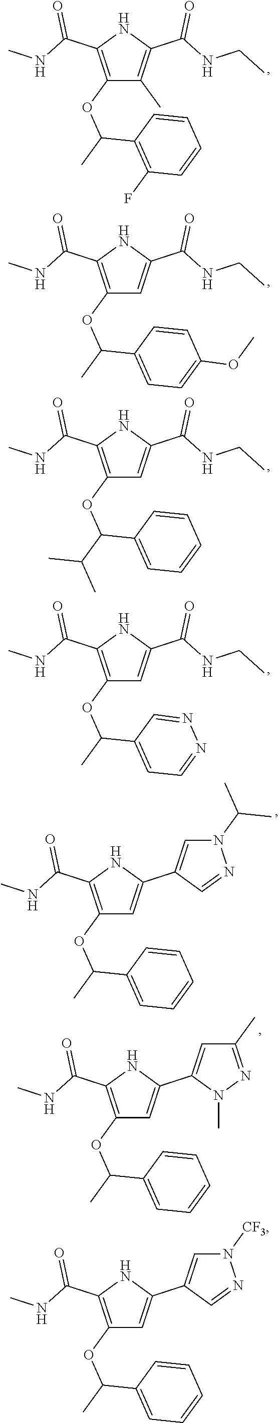

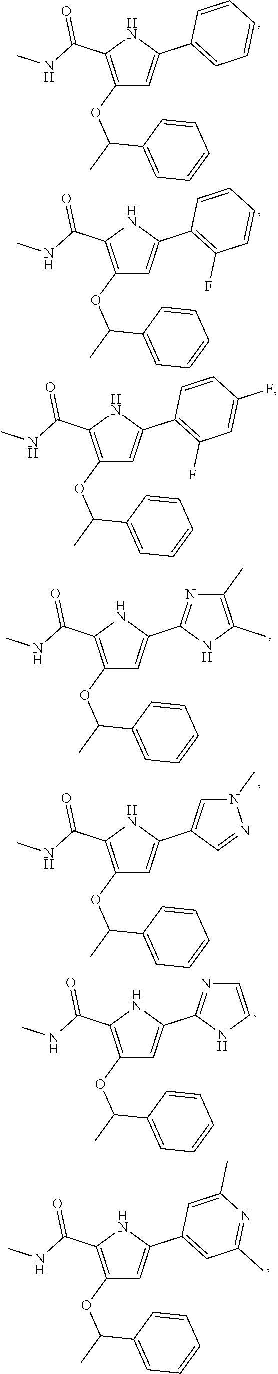

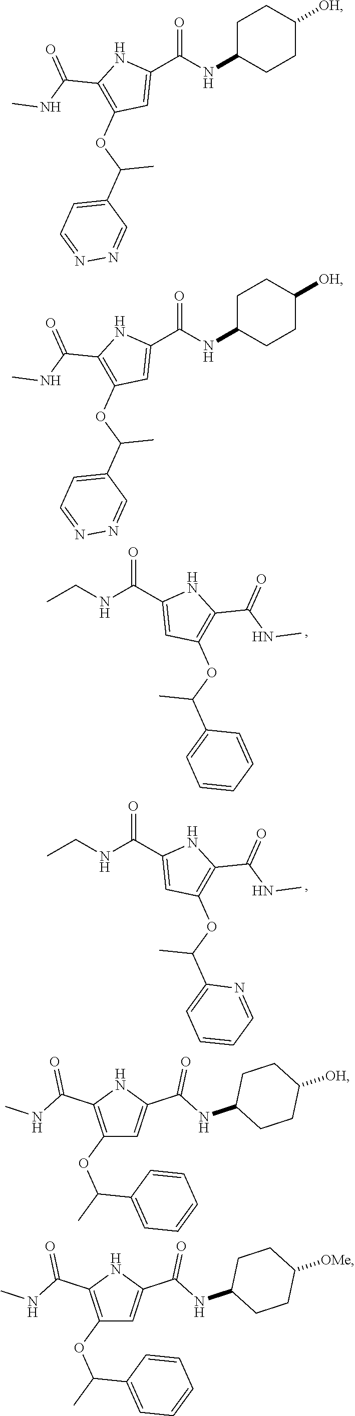

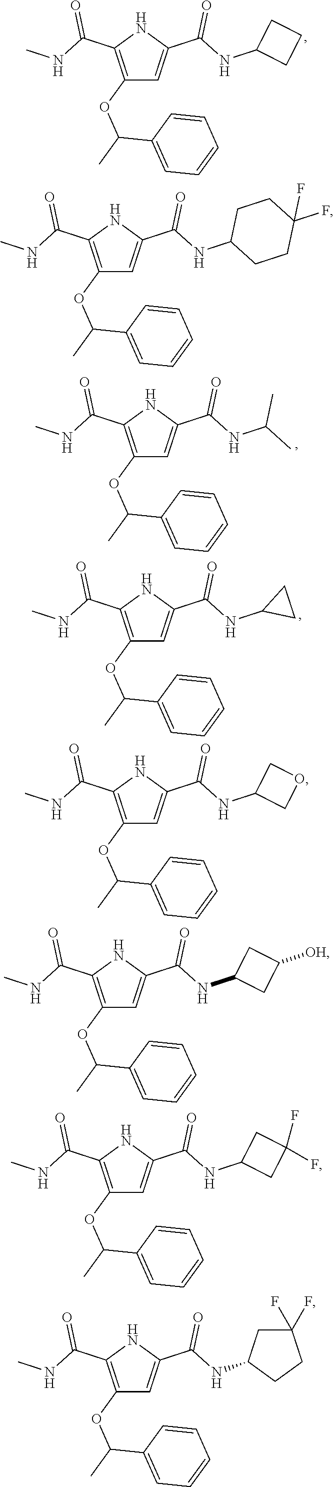

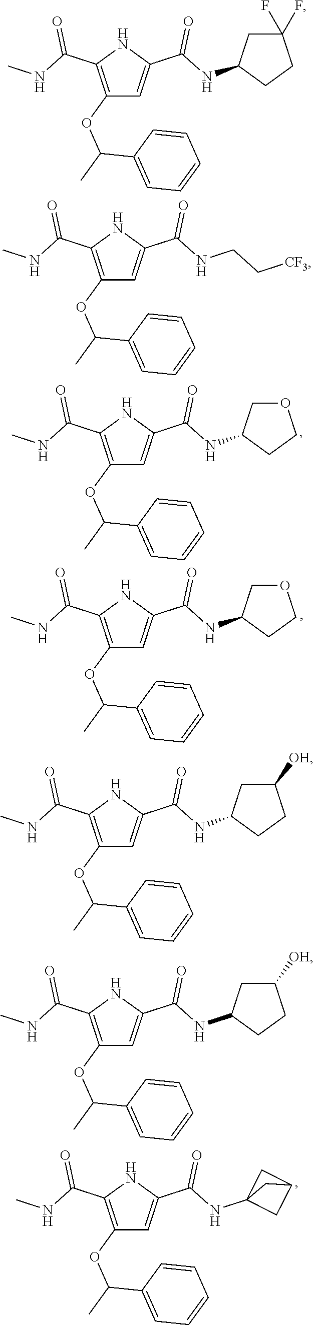

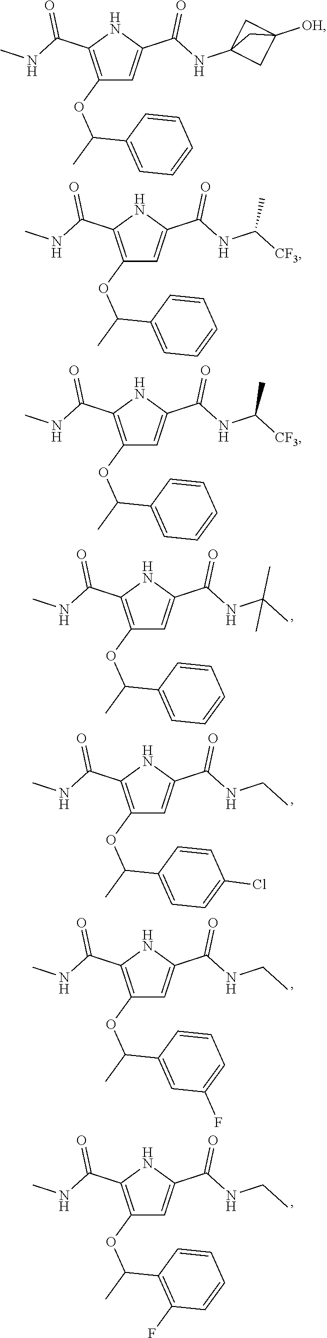

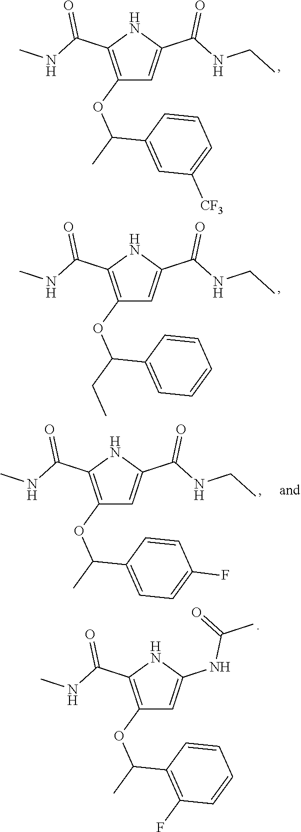

In an embodiment, the compound according to formula (I) or formula (IIA) is selected from:

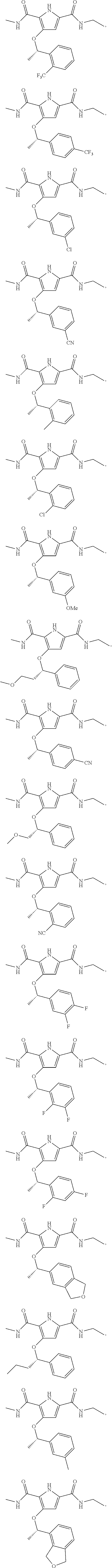

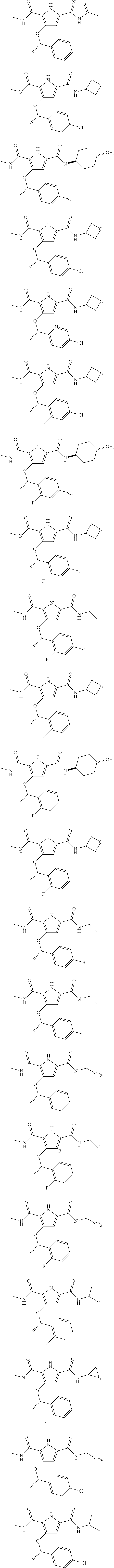

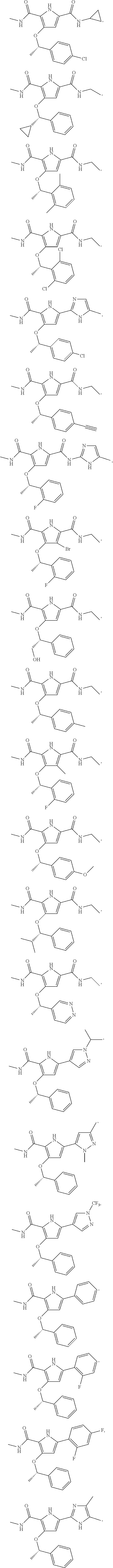

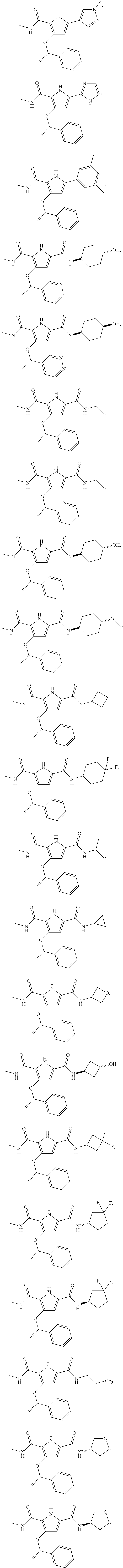

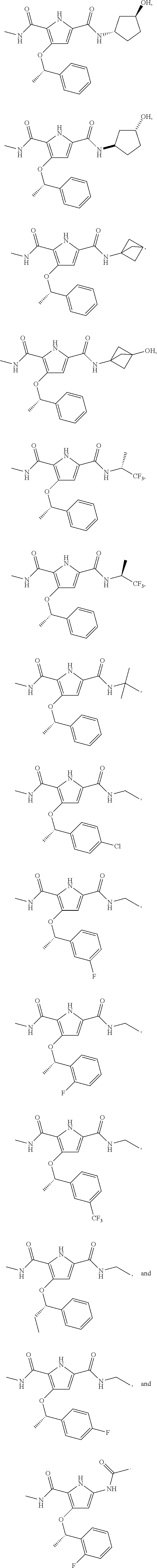

This invention also encompasses stereoisomers of any of the foregoing.

In an embodiment, the compound according to formula (I) or formula (IIB) is selected from:

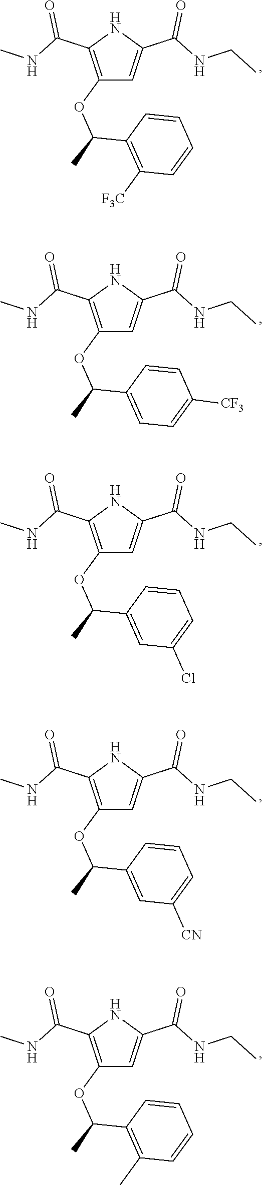

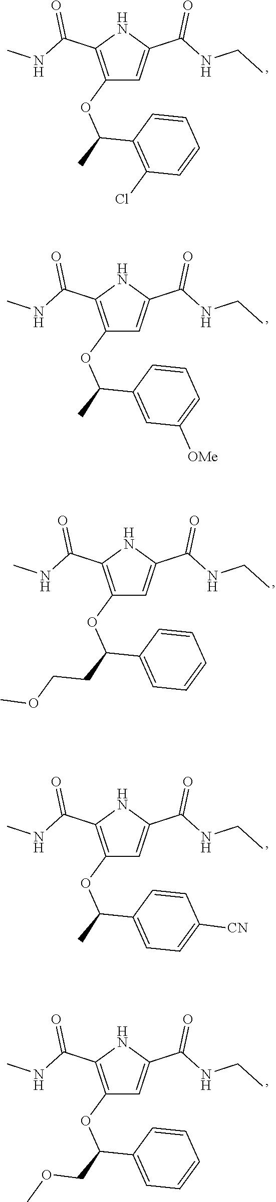

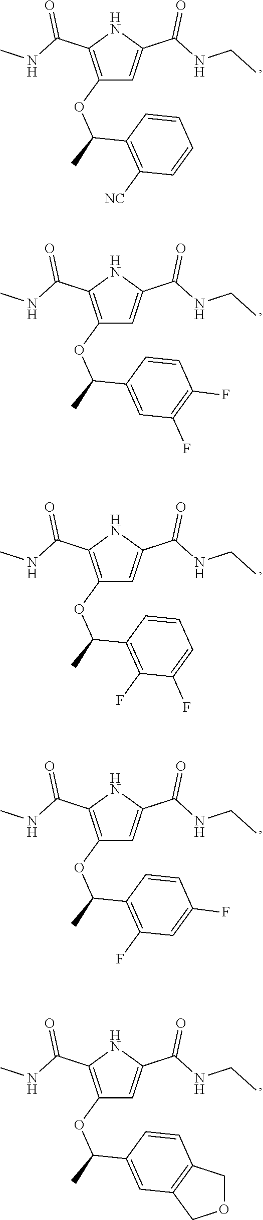

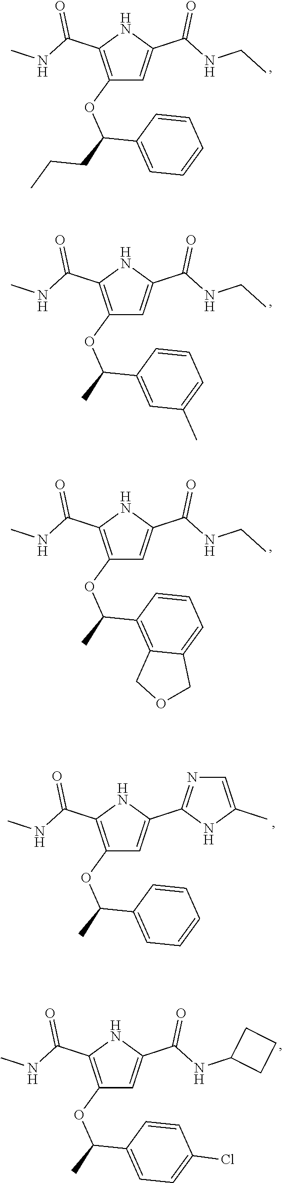

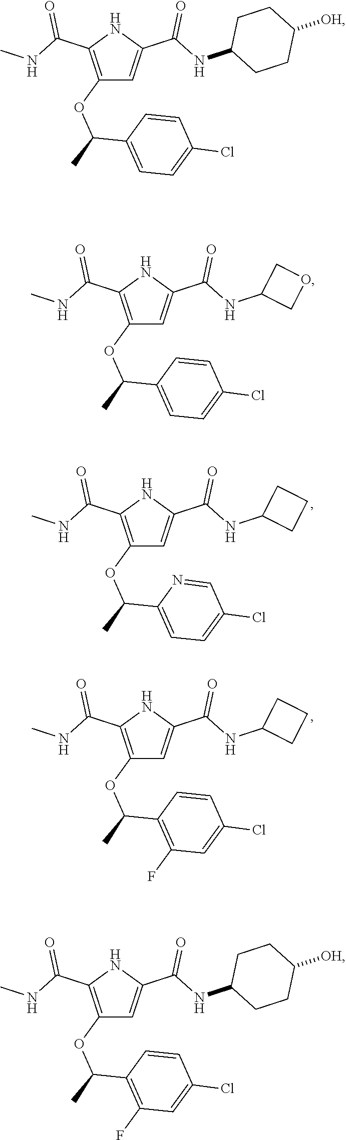

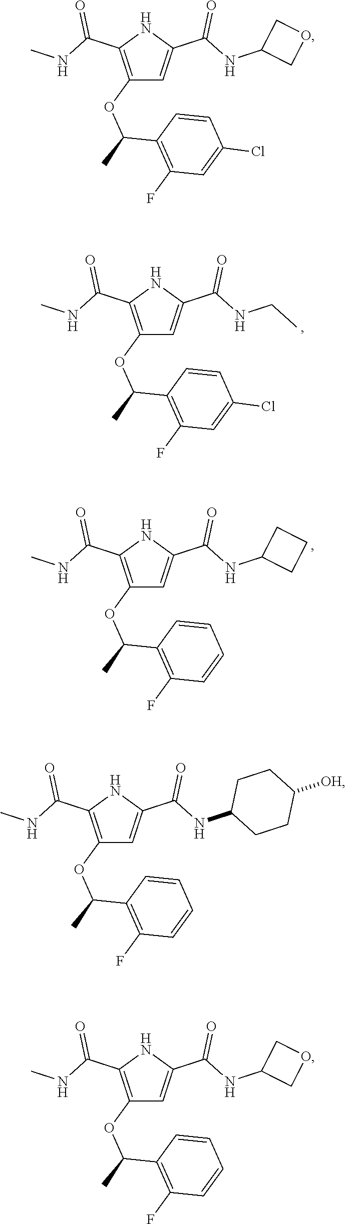

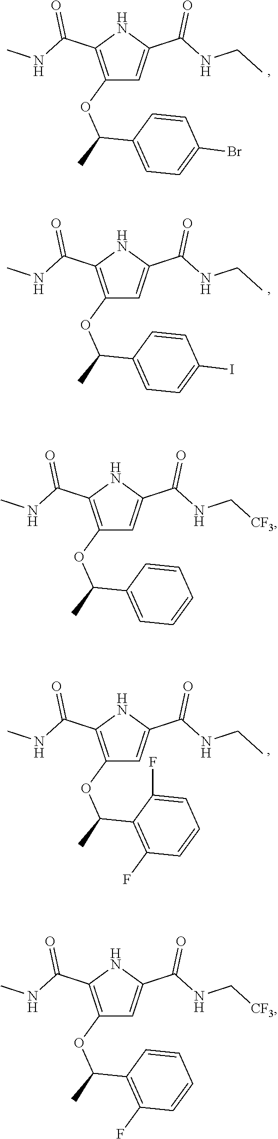

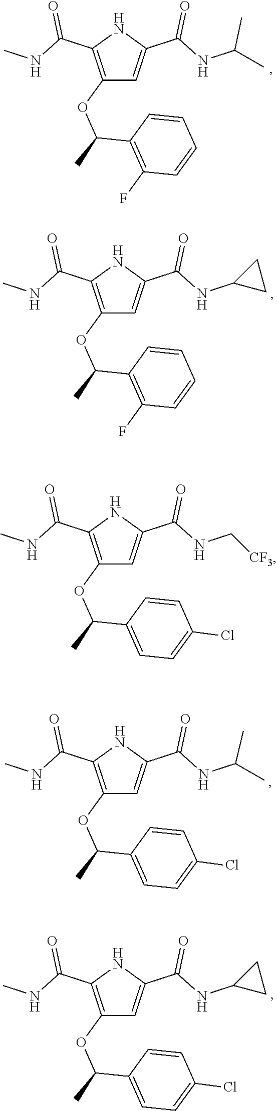

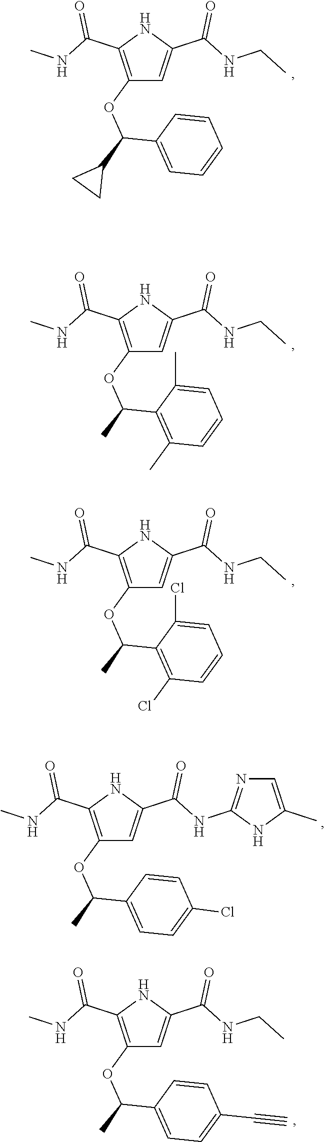

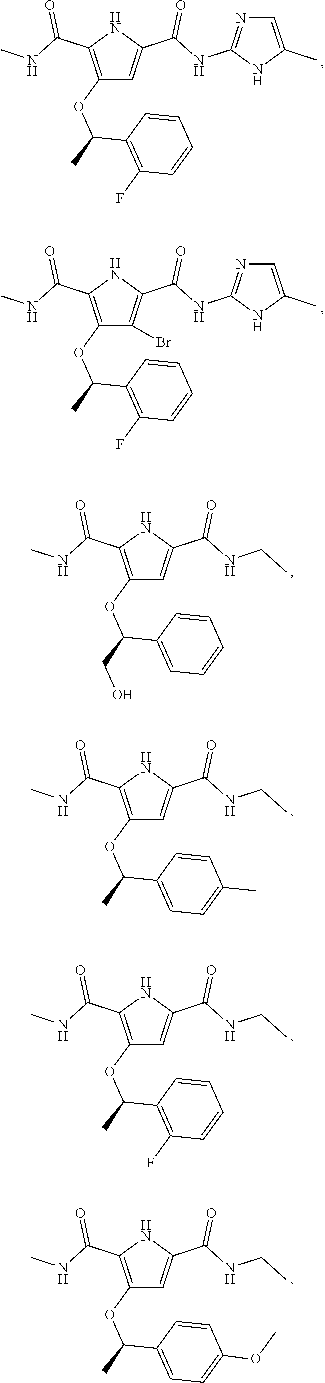

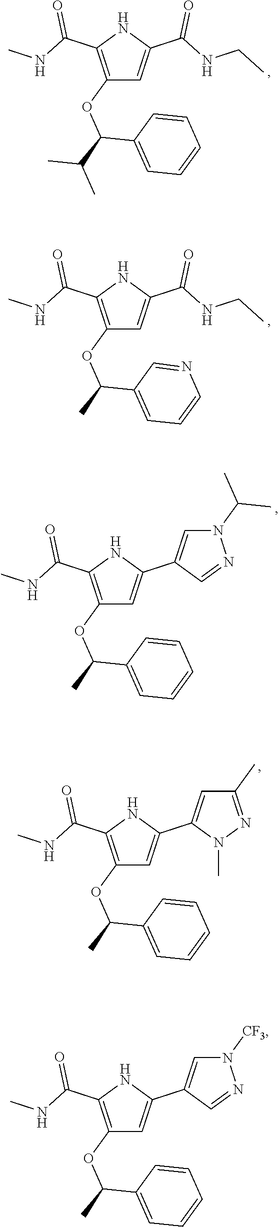

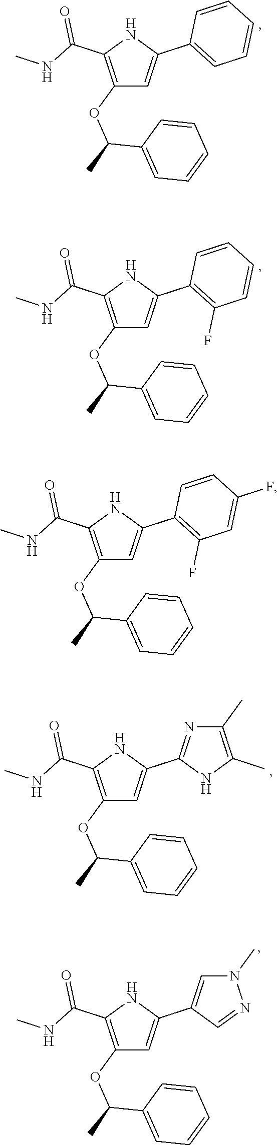

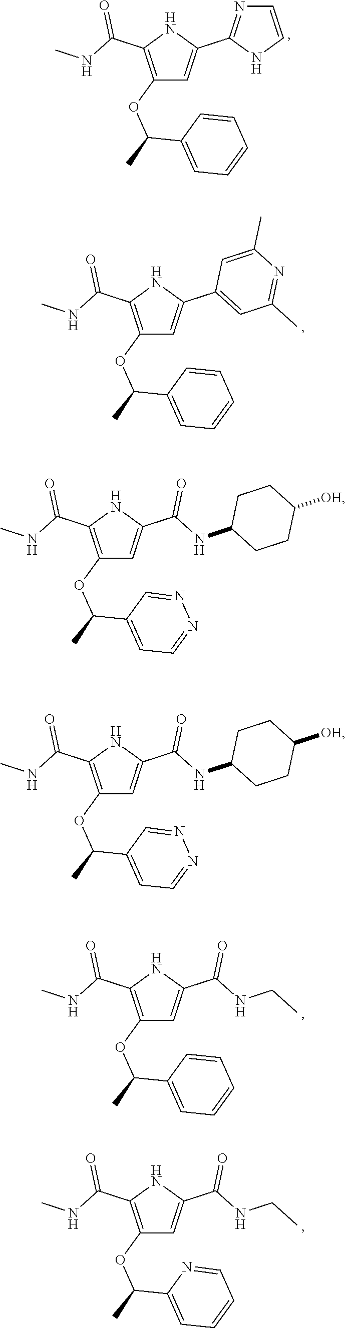

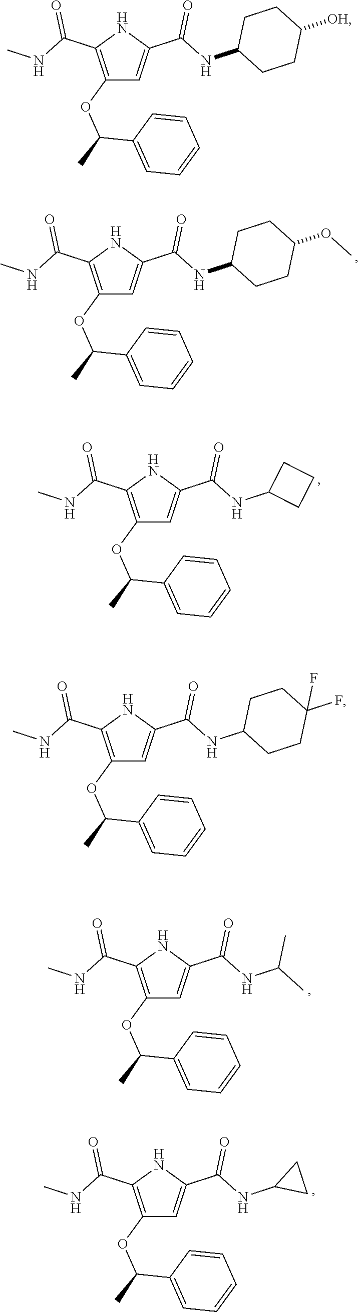

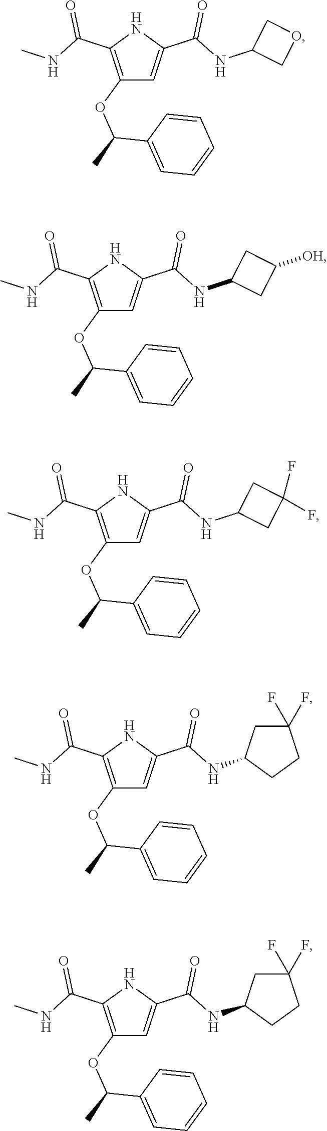

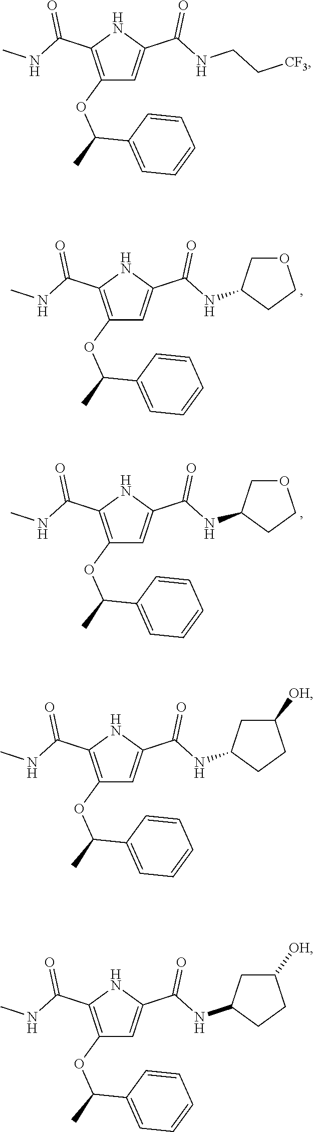

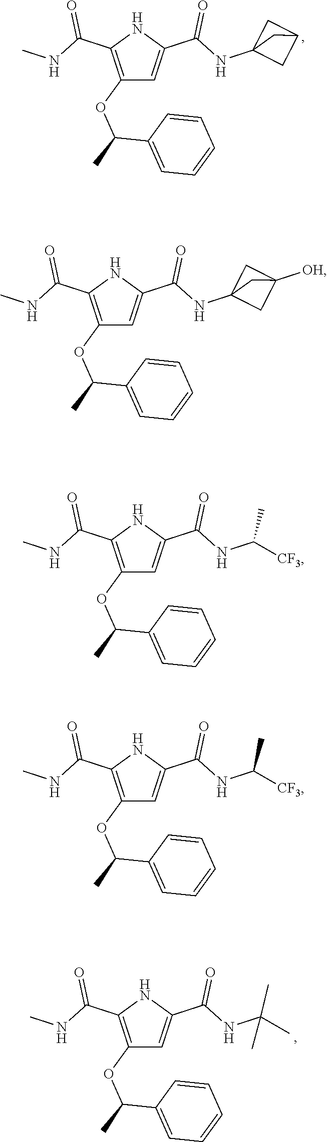 [SMILES: CNC(=O)c1[nH]c(C(=O)NC23CC(C2)C3)cc1O[C@H](C)c1ccccc1 ; CNC(=O)c1[nH]c(C(=O)NC23CC(O)(C2)C3)cc1O[C@H](C)c1ccccc1 ; CNC(=O)c1[nH]c(C(=O)N[C@H](C)C(F)(F)F)cc1O[C@H](C)c1ccccc1 ; CNC(=O)c1[nH]c(C(=O)N[C@@H](C)C(F)(F)F)cc1O[C@H](C)c1ccccc1 ; CNC(=O)c1[nH]c(C(=O)NC(C)(C)C)cc1O[C@H](C)c1ccccc1]

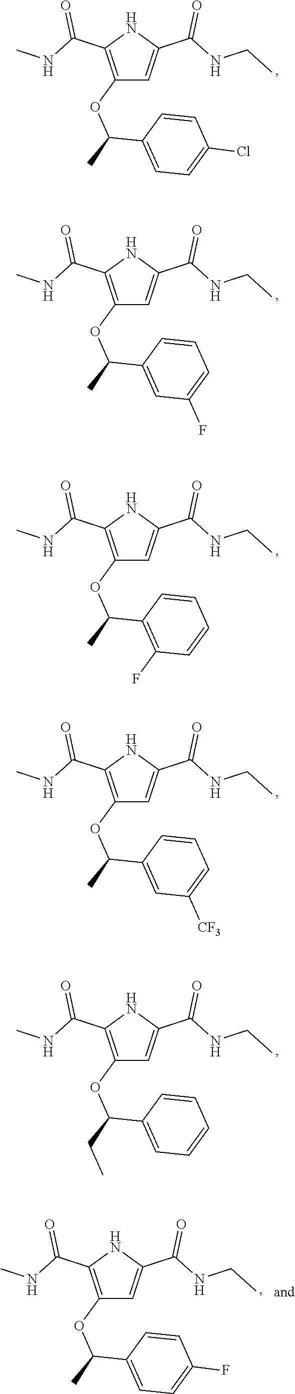

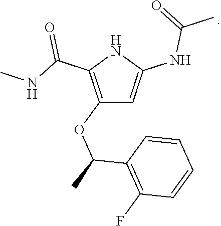 [SMILES: CNC(=O)c1[nH]c(NC(C)=O)cc1O[C@H](C)c1ccccc1F]

This invention also encompasses stereoisomers of any of the foregoing.

In the preceding three paragraphs, and throughout the specification, a down or up wedge bond (i.e., [symbol] or [symbol] ) is used to depict absolute configuration. Where a down or up wedge bond is used at a particular position, the compound has substantially a single configuration (either R or S) at the indicated position. A down or up rectangular bond (i.e., [symbol] or [symbol] ) is used to depict relative stereochemistry between two positions. Where a down or up rectangular bond is used at a chiral centre, the compound is in the form of a mixture (typically a 1:1 mixture) of R and S configurations at the indicated position.

In some embodiments, the present disclosure is directed to methods of using a compound of formula (I), or a tautomer, a stereoisomer or a mixture of stereoisomers, a pharmaceutically acceptable salt, a hydrate, a deuterated derivative, or an N-oxide thereof:

[symbol]

wherein Ring A, X, Z, R^{1a}, R^{1b}, R², R³, R⁴ and m are as herein described.

In some embodiments, the compound of formula (I) is a compound of formula (IIA), or a tautomer, a stereoisomer or a mixture of stereoisomers, a pharmaceutically acceptable salt, a hydrate, a deuterated derivative, or an N-oxide thereof:

[symbol]

wherein Ring A, X, Z, R^{1a}, R^{1b}, R², R³, R⁴ and m are as herein described.

In some embodiments, the compound of formula (I) is a compound of formula (IIB), or a tautomer, a stereoisomer or a mixture of stereoisomers, a pharmaceutically acceptable salt, a hydrate, a deuterated derivative, or an N-oxide thereof:

[symbol]

wherein Ring A, X, Z, R^{1a}, R^{1b}, R², R³, R⁴ and m are as herein described.

In some embodiments, the compound of formula (I) is a compound of formula (XIIIA), or a tautomer, a stereoisomer or a mixture of stereoisomers, a pharmaceutically acceptable salt, a hydrate, a deuterated derivative, or an N-oxide thereof:

[symbol]

wherein R^{1a}, R^{2a}, R^{2b}, R³, R⁴, R¹⁰ and m are as herein described.

In some embodiments, the compound of formula (I) is a compound of formula (XIIIB), or a tautomer, a stereoisomer or a mixture of stereoisomers, a pharmaceutically acceptable salt, a hydrate, a deuterated derivative, or an N-oxide thereof:

[symbol]

wherein R^{1a}, R^{2a}, R^{2b}, R³, R⁴, R¹⁰ and m are as herein described.

In some embodiments, the compound of formula (I) is selected from Examples 1-128 as disclosed herein, or a tautomer, a stereoisomer or a mixture of stereoisomers, a pharmaceutically acceptable salt, a hydrate, a deuterated derivative, or an N-oxide thereof.

In some embodiments, the present disclosure provides methods of using a pharmaceutical composition comprising a compound disclosed herein, and one or more pharmaceutically acceptable excipients.

In accordance with a second aspect, the present disclosure provides a pharmaceutical composition comprising a compound defined in the first aspect, and one or more pharmaceutically acceptable excipients.

In accordance with a third aspect, the present disclosure provides a compound as defined in the first aspect or a pharmaceutical composition as defined in the second aspect, for use as a medicament.

In accordance with a fourth aspect, the present disclosure provides the use of a compound as defined in the first aspect or a pharmaceutical composition as defined in the second aspect, for the manufacture of a medicament.

In accordance with a fifth aspect, the present disclosure provides a compound as defined in the first aspect, or a pharmaceutical composition as defined in the second aspect, for use in a method of treatment or prophylaxis of an inflammatory disease, e.g., inflammatory skin disorders, respiratory diseases, gastrointestinal diseases, eye diseases, cancers, rheumatic diseases, demyelinating diseases and fibrotic diseases.

In accordance with a sixth aspect, the present disclosure provides a method for the treatment or prophylaxis of an inflammatory disease, e.g., inflammatory skin disorders, respiratory diseases, gastrointestinal diseases, eye diseases, cancers, rheumatic diseases, demyelinating diseases, and fibrotic diseases, said method comprising administering to a subject, an effective amount of a compound as defined in the first aspect, or a pharmaceutical composition as defined in the second aspect.

In accordance with a seventh aspect, the present disclosure provides the use of a compound as defined in the first aspect, or a pharmaceutical composition as defined in the second aspect for the manufacture of a medicament for the treatment or prophylaxis of an inflammatory disease, e.g., inflammatory skin disorders, respiratory diseases, gastrointestinal diseases, eye diseases, cancers, rheumatic diseases, demyelinating diseases, and fibrotic diseases, said method comprising administering to a subject, an effective amount of a compound as defined in the first aspect, or a pharmaceutical composition as defined in the second aspect.

In accordance with an eighth aspect, the present disclosure provides a method of inhibiting Bromodomain and Extra-Terminal protein activity in a subject, said method comprising administering to a subject an effective amount of a compound as defined in the first aspect, or a pharmaceutical composition as defined in the second aspect.

In accordance with a ninth aspect, the present disclosure a method of treating a disorder associated with Bromodomain and Extra-Terminal protein activity in a subject, said method comprising administering to a subject an effective amount of a compound as defined in the first aspect, or a pharmaceutical composition as defined in the second aspect.

Additional aspects of the present disclosure are disclosed throughout the specification.

Selective BET BDII inhibitors, such as the compounds disclosed herein, may in one or more embodiments, be of value and used in the treatment or amelioration of the following non-limiting examples of disorders and diseases.

The present disclosure provides selective BET inhibitors that can provide new and effective treatment and relief for joint related diseases and disorders. Joints may be infected by many types of microorganisms (bacteria, fungi, viruses) and occasionally by animal parasites. Infection related joint diseases and disorders include infection by direct contamination, by way of the bloodstream e.g., through the synovial blood vessels, and by extension from adjacent bony infections (osteomyelitis). Infectious arthritis may affect one joint (monarthritis) or a few joints (oligoarthritis) rather than many (polyarthritis). Joints or parts thereof can be damaged e.g., cartilage by for example through staphylococci, hemolytic streptococci, and pneumococci infections, e.g., bone through tuberculosis such as tuberculous spondylitis (Pott disease), or through Coccidioides immitis, brucellosis, such as Brucella suis, leprosy (Hansen disease), rubella (German measles) and serum hepatitis, viral synovitis, dranunculiasis (Guinea worm disease), sexually transmitted diseases, including gonorrhea, reactive arthritis (Reiter disease), congenital syphilis such as Clutton joint lesion, and Yaws, which leads to skeletal lesions. Inflammation may destroy the joint cartilage and underlying bone and cause irreparable deformities. Adhesions between the articulating members are frequent in such cases, and the resulting fusion with loss of mobility is called ankylosis such as ankylosing spondylitis, (Marie-Strümpell disease or Bechterew disease). Another type of arthritis is associated with chronic intestinal diseases—ulcerative colitis, regional enteritis, inflammatory bowel disease, cirrhosis, and Whipple disease.

In addition to joint disorders and diseases resulting from any of the above, the present disclosure provides potent and selective BET inhibitors (e.g., compounds of formula (I)) that may also provide new and effective treatment or relief for noninflammatory joint diseases, injury and degenerative disorders. Trauma to joints includes blunt injuries, mild sprains, fractures and dislocations. ligamentous, tendinous, and capsular tears, tears in the semilunar cartilages (menisci), and hemarthrosis. Degenerative joint disease includes osteoarthritis, arthrosis deformans, precocious osteoarthritis congenital dysplasia malum coxae senilis, spondylosis, chrondromalacia patellae, metabolic diseases such gouty arthritis, podagra, ochronotic arthropathy, chondrocalcinosis, or pseudogout, mucopolysaccharidoses, Hurler syndrome, Morquio disease, and polyepiphyseal dysplasias.

The present disclosure provides potent and selective BET inhibitors (e.g., compounds of formula (I)) that may also provide new and effective treatment or relief for secondary joint diseases and disorders, including hemorrhagic joints, hemarthrosis, villonodular synovitis, joint diseases that arise in association with aseptic necrosis e.g., can occur with fractures, osteochondritis dissecans, slipped epiphysis, Osgood-Schlatter, Legg-Calve-Perthes, endocrine-malfunctioning resultant joint disorders, acromegaly, neurogenic arthropathy, Charcot joint, hypertrophic osteoarthropathy, reflex sympathetic dystrophy, joint tumors, synovial chondromatosis, cartilaginous nodules, synovial osteochondromatosism, synoviomas, synovial sarcomas, and polymyalgia rheumatica.

The present disclosure provides selective BET inhibitors that can provide new and effective treatment and relief for a fibrosis or fibrosis-associated condition. The present disclosure provides specific BET inhibitors that can retard the progression or severity of indicators of fibrosis e.g., kidney fibrosis.

The methods and compositions of the present disclosure can in some embodiments be useful therapeutically for a fibrosis or fibrosis-associated conditions affecting any tissue including, for example, fibrosis of an internal organ, a cutaneous or dermal fibrosing disorder, and fibrotic conditions of the eye. In some embodiments, the fibrosis or fibrosis-associated conditions include fibrosis of internal organs (e.g., liver, lung, kidney, heart blood vessels, gastrointestinal tract). In some embodiments, the fibrosis or fibrosis-associated conditions include pulmonary fibrosis, idiopathic fibrosis, autoimmune fibrosis, myelofibrosis, liver cirrhosis, veno-occlusive disease, mesangial proliferative glomerulonephritis, crescentic glomerulonephritis, diabetic nephropathy, renal interstitial fibrosis, renal fibrosis in subjects receiving cyclosporin, allograft rejection, HIV associated nephropathy. In some embodiments, the fibrosis-associated disorders include systemic sclerosis, eosinophilia-myalgia syndrome, and fibrosis-associated CNS disorders such as intraocular fibrosis. In some embodiments, dermal fibrosis disorders include, for example, scleroderma, morphea, keloids, hypertrophic scars, familial cutaneous collagenoma, and connective tissue nevi of the collagen type. In some embodiments, fibrotic conditions of the eye include conditions such as diabetic retinopathy, post-surgical scarring (for example, after glaucoma filtering surgery and after crossed-eyes (strabismus) surgery), and proliferative vitreoretinopathy. In some embodiments, fibrotic conditions that may be treated by the methods of the present invention may result, for example, from rheumatoid arthritis, diseases associated with prolonged joint pain and deteriorated joints, progressive systemic sclerosis, polymyositis, dermatomyositis, eosinophilic fascitis, morphea, Raynaud's syndrome, and nasal polyposis.

Organ disease often leads to organ fibrosis and which, in turn can lead to death. Fibrosis may follow a path independent of the organ. Fibrosis may be the result of excessive wound healing. In the kidney, this results mainly in glomerulosclerosis, tubular atrophy and dilation, tubulointerstitial fibrosis and capillary rarefaction. Renal fibrosis can be characterized by an excessive accumulation and deposition of extracellular matrix components. Renal fibrosis is not a simple, uniform scarring, but a dynamic process involving many, if not all, renal and infiltrating cell types. Kidneys often fail to repair themselves completely. Kidney cells can facilitate and increase the secretion of pro-fibrosis factors. When a normal healing response fails, scarring continues, and this can cause chronic kidney disease (CKD). Progressive scarring replaces normal kidney tissue with fibrotic tissue and kidney function is lost, which may lead to kidney failure. MMP-2, MCP-1 and TGF-β have been shown to identify patients with fibrosis and future poor renal outcomes.

The present disclosure provides specific BET inhibitors (e.g., Example 101) that have been found to be surprisingly effective against renal fibrosis and renal fibrosis-related conditions and or may provide a suitable treatment in limiting or slowing its progression. In some embodiments, the present disclosure provides potent and selective BET inhibitors (e.g., compounds of formula (I)) that can provide a new and effective treatment and relief for fibrosis and fibrosis-related conditions e.g., renal fibrosis and renal fibrosis-related conditions and/or limit or slow its progression. The present disclosure provides potent and selective BET inhibitors (e.g., compounds of formula (I)) that can provide new and effective treatment or relief for inflammatory fibrosis (e.g., renal fibrosis) and/or limit or slow its progression.

The present disclosure provides potent and selective BET inhibitors (e.g., compounds of formula (I)) that may also provide new and effective treatment or relief for noninflammatory fibrosis (e.g., renal fibrosis) diseases, injury, and degenerative disorders and/or limit or slow their progression.

Selective BET BDII inhibitors, such as the compounds disclosed herein, may in one or more embodiments, be of value and used in the treatment or amelioration of inflammatory disorders, immune disorders, and autoimmune disorders, which include diseases that have or may have an inflammatory or autoimmune component.

The inflammatory disorder, immune disorder, or autoimmune disorder may be a skin disorder selected from acne, inflammatory acne, acne fulminans, angiofibroma, nodular papulopustular acne, acne conglobata, acute erysipelas, alopecia, alopecia areata, alopecia totalis, atopic dermatitis, alopecia universalis, autoimmune bullous skin disorder such as pemphigus vulgaris (PV) or bullous pemphigoid (BP), bacterial skin infections, viral skin infections, bullous diseases, cellulitis, cutaneous abscesses, carbuncles, chronic hand eczema, cutaneous mastocytosis, Dercum disease, dermatological pain, dermatological inflammation, contact dermatitis, dermatitis, dermatitis herpetiformis, dermatomyositis, chronic atypical neutrophilic dermatosis with lipodystrophy and elevated temperature (CANDLE), neutrophilic dermatoses, such as pyoderma gangrenosum and Sweets syndrome, paronychial infections, pustulosis palmoplantaris edematous, erythema multiforme, erythema nodosum, granuloma annulare, pemphigus, epidermal necrolysis pemphigus, paraneoplastic pemphigus, erythrasma, ecthyma, eczema, folliculitis, furuncles, gustatory sweating, hyperhidrosis, Hailey-Hailey disease, hives, hidradenitis suppurativa, hypertrophic scars, impetigo, ichthyosis, ischemic necrosis, keloids, necrotizing subcutaneous infections, actinic keratosis, keratosis pilaris, miliaria, molluscum contagiosum, lichen planus, netherton syndrome, Pityriasis rubra pilaris, psoriasis, pruritus, prurigo nodularis, rashes, rosacea, pediculosis, Pityriasis rosea, scleroderma, scalded skin syndrome, skin rash, skin irritation, skin sensitization (e.g., contact dermatitis or allergic contact dermatitis), trauma or injury to the skin, post-operative or post-surgical skin conditions, wounds, burns (including chemical, electrical fire, friction, radiation, temperature related, thermal and cold), sunburn, scarring, scabies, skin ulcers, urticaria pigmentosa, urticarial and chronic idiopathic pruritus, vitiligo, warts, and xerosis.

The inflammatory disorder, immune disorder, or autoimmune disorder may be a respiratory disease selected from asthma, bronchiectasis, bronchiolitis, byssinosis, chronic obstructive pulmonary disease (COPD), fibrosis, cystic fibrosis, hypersensitivity pneumonitis, mesothelioma, pneumoconiosis, (idiopathic) pulmonary fibrosis, rhinitis, rhinosinusitis and sarcoidosis.

The inflammatory disorder, immune disorder, or autoimmune disorder may be a gastrointestinal disease selected from celiac disease, Crohn's disease, eosinophilic esophagitis, inflammatory bowel disease, retroperitoneal fibrosis, and ulcerative colitis.

The inflammatory disorder, immune disorder, or autoimmune disorder may be an eye disease selected from conjunctivitis, dry eye syndrome, iritis, keratitis, macular degeneration, myasthenia gravis, scleritis, Sjögran's syndrome, and uveitis.

The inflammatory disorder, immune disorder, or autoimmune disorder may be a cardiovascular disease or associated disorder, selected from cerebrovascular disease, aorta disease, arrhythmias, atherosclerosis, aneurysm, angina, stroke, carditis, cardiac hypertrophy, cardiomyopathy, endocarditis, coronary artery disease, deep vein thrombosis, heart attack, heart disease, heart failure, Marfan syndrome, myocarditis, peripheral artery disease, pericarditis. pulmonary embolism, rheumatic heart disease, thrombosis, valvular heart disease, ventricular heart disease, ventricle dysfunction, and vascular diseases.

The inflammatory disorder, immune disorder, or autoimmune disorder may be a systemic indication selected from Addison's disease, AIDS, ankylosing spondylitis, atherosclerosis, arthritis, Behcet's disease, cryopyrin-associated periodic syndromes (CAPS), chronic kidney diseases (including, but not limited to nephritis, nephropathy, hypertensive nephropathy, HIV-associated nephropathy, IgA nephropathy, familial Mediterranean fever, focal segmental glomerulosclerosis, Grave's disease, juvenile arthritis, lymphangitis, lymphadenitis, lupus nephritis, minimal change disease, neurofibromatoses, polycystic kidney disease and tubular interstitial nephritis), acute kidney injury disease or condition (including, but are not limited to ischemia-reperfusion induced, cardiac and major surgery induced, percutaneous coronary intervention induced, radio-contrast agent induced, sepsis induced, pneumonia induced, and drug toxicity induced), giant cell arthritis, glomerulonephritis, gout, hepatitis, hepatitis B, hepatitis C, hypophysitis, Kawasaki disease, liver fibrosis, multiple sclerosis, myositis, osteoarthritis, pancreatitis, pneumonitis, polyarteritis nodosa, primary biliary cirrhosis, prostate disease, prostatitis, benign prostatic hyperplasia (BPH), psoriatic arthritis, rheumatoid arthritis, scleritis, scleroderma (cutaneous or systemic), sclerosing cholangitis, sepsis, systemic lupus erythematosus, systemic mastocytosis, Takayasu's arteritis, thyroiditis, toxic shock, vasculitis, warm autoimmune hemolytic anemia, and Wegener's granulomatosis.

The inflammatory disorder, immune disorder, or autoimmune disorder may be an autoimmune disease or indication where immunosuppression would be desirable, for instance, to avoid organ transplant rejection and graft versus host disease (chronic or acute).

Selective BET BDII inhibitors, such as the compounds disclosed herein, may in one or more embodiments, be of value and used in the treatment or amelioration of cancers.

The cancer may be a skin or systemic cancer, selected from acoustic neuroma, anal cancer, bladder cancer, Bowen's disease, brain cancer, breast cancer, carcinomas including basal cell carcinoma, bile duct carcinoma, bronchogenic carcinoma, choriocarcinoma, embryonal carcinoma, cystadenocarcinoma, epithelial carcinoma, medullary carcinoma, NUT midline carcinoma (NMC), papillary carcinoma, papillary adenocarcinomas, renal cell carcinoma, sebaceous gland carcinoma, small cell lung carcinoma, squamous cell carcinoma, and sweat gland carcinoma, cervical cancer, chordoma, colon cancer, colorectal cancer, craniopharyngioma, dysproliferative changes (dysplasias and metaplasias), endometrial cancer, ependymoma, esophageal cancer, essential thrombocythemia, estrogen-receptor positive breast cancer, Ewing's tumour, genital cancer, cancer of the cervix, cancer of the vulva, vulvar intraepithelial neoplasia (VIN), cancer of the vagina, germ cell testicular cancer, gastrointestinal cancers, gastric cancer, glioblastoma, glioma, heavy chain disease, hemangioblastoma, hepatocellular cancer, hepatoma, hormone insensitive prostate cancer, keratinocyte carcinomas, kidney cancer, leukaemias including acute leukaemia, acute lymphocytic leukaemia, acute myeloid leukaemia, acute myelocytic leukaemia (monocyctic, myeloblastic, adenocarcinoma, angiosarcoma, astrocytoma, myelomonocytic and promyelocytic), acute t-cell leukaemia, chronic leukaemia, chronic lymphocytic leukaemia, chronic myelocytic (granulocytic) leukaemia, chronic myelogenous leukaemia, erythroleukemia, lymphoblastic leukaemia, and myelogenous leukaemia, liver cancer, lung cancer, lymphoid malignancies of T-cell or B-cell origin, lymphomas (Hodgkin's and non-Hodgkin's) including cutaneous T-cell lymphoma, diffuse large B-cell lymphoma, and follicular lymphoma, cutaneous (skin) lymphomas, malignancies and hyperproliferative disorders including of the bladder, breast, colon, lung, ovaries, pancreas, prostate, skin and uterus, advanced malignancies, medulloblastoma, melanoma, meningioma, Merkel cell cancer mesothelioma, metastatic cancer, multiple myeloma, myeloma, pancreatic cancer, myelofibrosis, myeloproliferative neoplasms, neuroblastoma, non-small cell lung cancer, head and neck cancer, oligodendroglioma, oral cancer, ovarian cancer, pancreatic cancer, pinealoma, polycythemia vera, prostate cancer, rectal cancer, retinoblastoma, sarcomas including chondrosarcoma, endotheliosarcoma, fibrosarcoma, gliosarcoma, leiomyosarcoma, liposarcoma, lymphagioendotheliosarcoma, lymphangiosarcoma, myxosarcoma, Castleman's disease and Kaposi's sarcoma, osteogenic sarcoma, and rhabdomyosarcoma, seminoma, skin cancer, skin adnexal tumors, and sarcomas, small cell lung cancer, solid tumors, stomach cancer, synovioma, testicular tumours, thyroid cancer, uterine cancer, Waldenstrom's macroglobulinemia, and Wilms' tumour.

Selective BET BDII inhibitors, such as the compounds disclosed herein, may in one or more embodiments, be used to provide male contraception.

Selective BET BDII inhibitors, such as the compounds disclosed herein, may in one or more embodiments, be of use in the treatment or amelioration of obesity, dyslipidaemia, cholesteatoma, hypercholesterolemia, Alzheimer's disease, metabolic syndrome, hepatic steatosis, type I diabetes, type II diabetes, and complications from diabetes, insulin resistance, and diabetic retinopathy or diabetic neuropathy.

Selective BET BDII inhibitors, such as the compounds disclosed herein, may in one or more embodiments, be of use in the treatment or amelioration of an immune system dysfunction, a viral disease, a bacterial disease, a yeast disease, non-inflammatory acne, an allergic disease, asthma, food allergy, rhinitis, an IL-6 pathway-related disease, an immune response, and a hyperproliferative disorder;

Selective BET BDII inhibitors, such as the compounds disclosed herein, may in one or more embodiments, be of use in the treatment or amelioration of Aicardi-Goutières syndrome, chilblain lupus, stimulator of interferon genes-Associated Vasculopathy with onset in Infancy (SAVI), Singleton-Merten syndrome, retinal vasculopathy with cerebral leukodystrophy, autoimmune uveitis, lupus, systemic sclerosis, an autoimmune thyroid disease, an allograft rejection, a graft-versus-host disease, an allograft rejection reaction, and a graft-versus-host reaction.

Selective BET BDII inhibitors, such as the compounds disclosed herein, may in one or more embodiments, be of use in the treatment or amelioration of disorders caused by a virus, such as Epstein-Barr virus (EBV), HIV, HTLV 1, chickenpox, herpes simplex virus infections, herpes zoster virus (VZV), and human papillomavirus (HPV) disease.

Selective BET BDII inhibitors, such as the compounds disclosed herein, may in one or more embodiments, be of use in the treatment or amelioration of mucopurulent cervicitis (MPC), urethritis, nongonococcal urethritis (NGU), vulvar disorders, vulvodynia, vulvar pain, vulvar dystrophy, pelvic inflammation, endometritis, salpingitis, oophoritis, dyspareunia, anal and rectal disease, anal abscess/fistula, anal fissure, anal warts, hemorrhoids, anal itch, pruritus ani, fecal incontinence, constipation, and polyps of the colon and rectum.

Selective BET BDII inhibitors, such as the compounds disclosed herein, may in one or more embodiments, be of use in the restoration of integrity or acceleration of the restoration of the integrity of an area of broken or damaged tissue, skin or mucosa, and in the reduction and amelioration of scar formation or scars.

Selective BET BDII inhibitors, such as the compounds disclosed herein, may in one or more embodiments, be of use in the treatment or amelioration of pyoderma gangrenosum (PG), palmar plantar pustulosis (PPP), and generalized pustular psoriasis (GPP).

Selective BET BDII inhibitors, such as the compounds disclosed herein, may in one or more embodiments, be of use in the treatment or amelioration of Crohn's disease, multiple sclerosis, rheumatoid arthritis, rhinosinusitis, and ulcerative colitis.

Selective BET BDII inhibitors, such as the compounds disclosed herein, may in one or more embodiments, be of use in the treatment or amelioration of cryopyrin-associated periodic syndromes (CAPS), cardiovascular disease, cerebrovascular disease, familial mediterranean fever, Grave's disease, liver fibrosis, neurofibromatoses, myocarditis, pericarditis, prostate disease, prostatitis, benign prostatic hyperplasia (BPH), systemic mastocytosis, and warm autoimmune hemolytic anemia.

Selective BET BDII inhibitors, such as the compounds disclosed herein, may in one or more embodiments, be of use in the treatment or amelioration of angiofibroma, chronic hand eczema, cutaneous mastocytosis, urticaria pigmentosa, neutrophilic dermatoses such as pyoderma gangrenosum and Sweets syndrome, chronic atypical neutrophilic dermatosis with lipodystrophy and elevated temperature (CANDLE), ichthyosis, keloids, scars, hypertrophic scars, netherton syndrome, pruritus, prurigo nodularis, and urticaria pigmentosa.

Selective BET BDII inhibitors, such as the compounds disclosed herein, may in one or more embodiments, also be of value and used in the palliation, diagnosis or prevention of any disease, disorder or condition in humans of one or more of the aforesaid non-limiting examples of disorders and diseases.

In some embodiments, use of the compounds to treat a disease as disclosed herein results in a therapeutic effect associated with a reduction in disease. In some embodiments, use of the compounds to treat a disease or disorder as disclosed herein results in a reduction of one or more tissue inflammation biomarkers selected from Col1A, TGF-b1, MCP-1, IL-1b, IL-6, IL-17, TNF-a, and Timp1. In some embodiments, use of the compounds to treat a disease or disorder as disclosed herein results in a reduction of one or more tissue inflammation biomarkers selected from Col1A, TGF-b1, MCP-1, IL-1b, IL-6, and Timp1. In some embodiments, use of the compounds to treat a disease or disorder as herein disclosed results in IL-17 and/or TNF-a being relatively unchanged. In some embodiments, use of the compounds to treat a disease or disorder as herein disclosed results in a small reduction in IL-17 and/or TNF-a,

Treatment or amelioration with selective BET BDII inhibitors, such as compositions comprising the compounds disclosed herein or salts thereof (or combinations thereof), in some embodiments may be effective if applied orally, in some other embodiments may be effective if applied by injection, in some other embodiments may be effective if applied topically, and in some further embodiments may be effective if applied topically and orally or by injection and topically or by orally and injection. In one or more embodiments treatment or amelioration with selective BET BDII inhibitors, such as compositions comprising the compounds disclosed herein or salts thereof (or combinations thereof), may be effective orally where the compounds have good bioavailability e.g., >about 25%. In some embodiments bioavailability is between about 20% to about 70%, or between about 20% to about 50%, or between about 20% to about 30%.

In some embodiments one or more compounds disclosed herein are applied orally, for example as a solid dose form e.g., as a tablet, or a capsule, or as a semisolid or fluid dose form e.g., as a gel, or as liquid. In a fluid or semisolid dosage form the compound may in one or more embodiments be delivered as a suspension or as a solution.

In some embodiments one or more compounds disclosed herein are applied by injection, e.g., as a solution or as a suspension. The solution or suspension may be in one or more embodiments, e.g., aqueous based, oil based, waterless, hydrophilic, hydrophobic, amphiphilic and or an emulsion.

In some embodiments one or more compounds disclosed herein are applied by inhalation, e.g., as a powder, spray or mist. In a fluid or liquid form, which can be used to form a mist (e.g., with a nebulizer) or spray (e.g., with an aerosol) the compound may in one or more embodiments be delivered as a suspension or as a solution.

In some embodiments one or more compounds disclosed herein are applied topically e.g., as a cream, emulsion, lotion, gel, ointment, mousse, foam, spray or other topical dosage formats known in the art. In some embodiments when applied topically, the compounds disclosed herein may be effective where the compound is delivered primarily or substantially into the skin with low levels of transdermal penetration. In some embodiments when applied topically the compounds disclosed herein may be effective where the compound is delivered primarily or substantially transdermally. In some embodiments when applied topically the compounds disclosed herein may be effective where the compound is delivered intradermally and transdermally. In some embodiments the penetration of the compound in the epidermis can be higher than that in the dermis. In some embodiments the penetration of the compound in the dermis can be higher than in the epidermis. In some embodiments the penetration of the compound in the dermis is similar to that in the epidermis. In some embodiments the concentration of the compound per unit volume in the epidermis can be higher than that in the dermis. In some embodiments the concentration of the compound per unit volume in the dermis can be higher than in the epidermis. In some embodiments the concentration of the compound per unit volume in the dermis is similar to that in the epidermis.

Compositions comprising a compound disclosed herein or salt thereof (or combinations thereof) may in one or more embodiments be administered buccally, by inhalation (e.g., spray, nebulizer, or powder puff), epidural, by injection (including intraarticular, intravenous, intracoronary, subcutaneous, intramyocardial, intraperitoneal, intramuscular, intravascular or infusion), intradermal, intraperitoneal, intrapulmonary, intraarticular (e.g., injection), nasally, orally, parenterally, rectally, sublingually, topically, transdermally, vaginally, or via an implanted reservoir.

Pharmaceutical compositions of the disclosure may be suitable for topical or transdermal administration.

Examples of dosage forms for topical or transdermal administration of a compound disclosed herein or salt thereof include creams, drops, lotions, emulsions, foams, gels, inhalants, mousses, ointments, pastes, patches, powders, solutions, or sprays.

In some embodiments the compound is micronized when provided as a powder or as a suspension. In some embodiments the compound comprises nanoparticles.

In some embodiments, compositions comprising a novel compound disclosed herein or salt thereof (or combinations thereof) may be administered to young children. In some embodiments, compositions comprising a compound of the disclosure or salt thereof (or combinations thereof) may be administered to adolescents or teenagers. In some embodiments, compositions comprising a compound of the disclosure or salt thereof (or combinations thereof) may be administered to adults.

In some embodiments, compounds of the disclosure exhibit one, two or more of the following characteristics ++++ or +++++ BRD4 BD2 IC₅₀, # or ## BRD4 BD1 IC₅₀ and XX or XXX fold. In some embodiments, compounds of the disclosure exhibit one, two or more of the following characteristics ++++ or +++++ BRD4 BD2 IC₅₀, # BRD4 BD1 IC₅₀ and XX or XXX fold. In some embodiments, compounds of the disclosure exhibit the following characteristics ++++ BRD4 BD2 IC₅₀, # BRD4 BD1 IC₅₀ and XX fold. In some embodiments, compounds of the disclosure exhibit the following characteristics +++++ BRD4 BD2 IC₅₀, # BRD4 BD1 IC₅₀ and XX fold. In some embodiments, compounds of the disclosure exhibit the following characteristics ++++ BRD4 BD2 IC₅₀, # BRD4 BD1 IC₅₀ and XXX fold. In some embodiments, compounds of the disclosure exhibit the following characteristics +++++ BRD4 BD2 IC₅₀, # BRD4 BD1 IC₅₀ and XXX fold.

In one or more embodiments, compounds of the disclosure exhibit a plasma stability of >about 80%, >about 85%, or >about 90% at 120 minutes. In some embodiments plasma stability is between about 85% to about 95%, or between about 75% to about 95% at 120 minutes.

In one or more embodiments compounds of the disclosure exhibit a microsomal half-life of >about 20, >about 30, >about 40, >about 50 or >about 60 minutes. In some embodiments microsomal half-life is between about 40 to about 70 minutes or between about 15 to about 70 minutes.

In one or more embodiments compounds of the disclosure exhibit a thermodynamic solubility in FaSSIF pH 6.5 buffer of >about 10, >about 50, >about 100, >about 150, or >about 200 μM. In some embodiments the thermodynamic solubility is between about 200 to about 1250 μM, or between about 5 to about 1250 μM.

In one or more embodiments, compounds of the disclosure exhibit a T_{1/20}f >about 0.25 hr, >about 0.5 hr, >about 1 hr, or >about 2 hr. In some embodiments the T_{1/2} is between about 0.5 hr. to about 2.5 hr., or between about 0.20 hr. and 2.5 hr.

In one or more embodiments compounds of the disclosure exhibit a bioavailability of >about 10%, >about 20%, >about 25%, >about 30%, >about 40%, >about 50%, >about 60%>about 70%, >about 80%, >about 90%, or >about 95. In some embodiments bioavailability is between about 20% to about 70%, or between about 20% to about 50%, or between about 20% to about 30%.

In one or more embodiments compounds of the disclosure are active against BRD4 BD2 and are selective for BD2 over BRD4 BD1. In one or more embodiments compounds of the disclosure may also exhibit two or more of the following: plasma stability, not rapidly cleared, thermodynamic solubility and bioavailability. In some embodiments a higher bioavailability can translate into a lower dosage and potentially fewer side effects e.g., in the alimentary canal. In one or more embodiments compounds of the disclosure exhibit a selectivity e.g., >about 35 Fold, >about 70 Fold, >about 100 Fold, >about 140 Fold, >about 200 Fold selectivity, or >about 250 Fold and or an IC₅₀ of e.g., <about 0.2 μM, <about 0.15 μM, <about 0.1 μM, <about 0.08 μM, <about 0.05 μM, < or about 0.04 μM for BRD4 BD2 and or a bioavailability of e.g., >about 25%>about 35%>about 45%, or >about 55% and or a plasma stability e.g., of >about 80%, >about 85%, or >about 90% at 120 minutes and or a microsomal half-life of e.g., >about 20, >about 30, >about 40, >about 50, or >about 60, minutes and or a thermodynamic solubility of e.g., >about 10, >about 50, >about 100, >about 150, or >about 200 μM. In some embodiments there is provided a range between any two numbers of the same type of measurement.

The invention may also be defined according to any one of the following numbered clauses:
1. A compound of formula (I), or a pharmaceutically acceptable salt or N-oxide thereof: [symbol]
wherein:
Ring A is independently selected from phenyl, 5-membered heterocyclyl, 6-membered heterocyclyl, 9-membered bicyclic heterocyclyl, and 10-membered bicyclic heterocyclyl;
X is independently selected from O and NR⁹;
Z is independently selected from N and CR¹⁰;
R^{1a} and R^{1b} are each independently selected from H, C₁-C₄-alkyl, C₂-C₄-alkenyl, C₂-C₄-alkynyl, C₁-C₄-haloalkyl, C₀-C₄-alkylene-R¹⁰, wherein R^{1c} is independently selected from C₃-C₆ cycloalkyl, 4-membered heterocyclyl, 5-membered heterocyclyl, and 6-membered heterocyclyl; wherein where R¹⁰ is C₃-C₆ cycloalkyl or a 5- to 10-membered heterocycloalkyl, R^{1c} is optionally substituted with from 1 to 4 R^{1d};
or R^{1a} and R^{1b} together with the nitrogen atom to which they are attached form a 5- to 8-membered heterocycloalkyl group optionally substituted with from 1 to 4 R^{1e};
R² is independently selected from —CONR^{2a}R^{2b}, 5-membered heterocyclyl, 6-membered heterocyclyl, and phenyl, wherein the 5-membered heterocyclyl, and 6-membered heterocyclyl groups may be optionally substituted with from 1 to 4 R^{2c} and wherein the phenyl group may be optionally substituted with from 1 to 5 R^{2c};
wherein R^{2a} and R^{2b} are each independently selected from H, C₁-C₄-alkyl, C₂-C₄-alkenyl, C₂-C₄-alkynyl, C₁-C₄-haloalkyl, C₀-C₄-alkylene-R^{2d}; wherein R^{2d} is independently selected from C₃-C₆ cycloalkyl, 4-membered heterocyclyl, 5-membered heterocyclyl, and 6-membered heterocyclyl, wherein where R^{2d} is C₃-C₆ cycloalkyl or a 5- to 10-membered heterocycloalkyl, R^{2d} is optionally substituted with from 1 to 4 R^{2e};
   or R^{2a} and R^{2b} together with the nitrogen atom to which they are attached form a 5- to 8-membered heterocycloalkyl group optionally substituted with from 1 to 4 R^{2f};
R^{1d}, R^{1e}, R^{2c}, R^{2e} and R^{2f} are each independently at each occurrence selected from ═O, ═S, halo, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₄-cycloalkyl, 4-membered heterocyclyl, 5-membered heterocyclyl, and 6-membered heterocycloalkyl;
R³ is independently selected from H, C₁-C₄-alkyl, C₂-C₄-alkenyl, C₂-C₄-alkynyl, C₁-C₄-haloalkyl, C₂-C₄-haloalkenyl, C₁-C₄-alkylene-OR⁷, C₀-C₄-alkylene-S(O)₂R⁶, C₀-C₄-alkylene-CONR⁶R⁶, C₃-C₄-cycloalkyl, 4-membered heterocyclyl, 5-membered heterocyclyl, and 6-membered heterocyclyl;
R⁴ is independently at each occurrence selected from ═O, ═S, halo, nitro, cyano, C₀-C₄-alkylene-NR⁵R⁶, C₀-C₄-alkylene-OR⁷, SR⁶, SOR⁶, C₀-C₄-alkylene-S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl, C₁-C₄-alkyl-S(O)₂R⁶, C₂-C₄-alkenyl, C₂-C₄-alkynyl, C₁-C₄-haloalkyl, C₃-C₄-cycloalkyl and 4-membered heterocycloalkyl;
R⁵ is independently at each occurrence selected from H, C₁-C₄-alkyl, C(O)—C₁-C₄-alkyl and S(O)₂—C₁-C₄-alkyl; and
R⁶ is independently at each occurrence selected from H and C₁-C₄-alkyl; or where two R⁶ groups are attached to the same nitrogen, those two R⁶ groups together with the nitrogen atom to which they are attached optionally form a 5- to 8-membered-heterocycloalkyl group optionally substituted with from 1 to 4 R⁸;
or R⁵ and R⁶ together with the nitrogen atom to which they are attached form a C₅-C₈-heterocycloalkyl group optionally substituted with from 1 to 4 R⁸;
R⁷ is independently at each occurrence selected from H, C₁-C₄-alkyl, C(O)—C₁-C₄-alkyl and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₄-cycloalkyl and 4-membered heterocycloalkyl;
R⁹ is independently selected from H, C₁-C₄-alkyl, C₂-C₄-alkenyl, C₂-C₄-alkynyl, C₁-C₄-haloalkyl, C₂-C₄-haloalkenyl and C₃-C₄-cycloalkyl;
R¹⁰ is independently selected from H, C₁-C₄-alkyl, C₂-C₄-alkenyl, C₂-C₄-alkynyl, C₁-C₄-haloalkyl, C₂-C₄-haloalkenyl, C₀-C₄-alkylene-OR⁷ and C₃-C₆-cycloalkyl; and
m is an integer selected from 0, 1, 2, 3 and 4;
wherein any of the aforementioned alkyl, alkylene, alkenyl, or cyclopropyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a}, and S(O)₂R^{a}; wherein R^{a} is independently at each occurrence selected from H and C₁-C₄-alkyl; and R^{b} is independently at each occurrence selected from H, C₁-C₄-alkyl, C(O)—C₁-C₄-alkyl and S(O)₂—C₁-C₄-alkyl.
2. The compound of clause 1, or a pharmaceutically acceptable salt or N-oxide thereof, having a structure according to Formula (IIA): [symbol]
3. A compound of clause 1 or clause 2, or a pharmaceutically acceptable salt or N-oxide thereof, wherein Ring A is 5-membered heteroaryl.
4. A compound of clause 1 or clause 2, or a pharmaceutically acceptable salt or N-oxide thereof, wherein when Ring A is phenyl.
5. A compound of any preceding clause, or a pharmaceutically acceptable salt or N-oxide thereof, wherein Z is CR¹⁰.
6. A compound of any preceding clause, or a pharmaceutically acceptable salt or N-oxide thereof, wherein Z is N.
7. A compound of any preceding clause, or a pharmaceutically acceptable salt or N-oxide thereof, wherein X is O.
8. A compound of any preceding clause, or a pharmaceutically acceptable salt or N-oxide thereof, wherein R^{1a} is C₁-C₄-alkyl and R^{1b} is H.
9. A compound of any preceding clause, or a pharmaceutically acceptable salt or N-oxide thereof, wherein R² is —CONR^{2a}R^{2b}.
10. A compound of clause 9, or a pharmaceutically acceptable salt or N-oxide thereof, wherein R^{2a} is C₁-C₄-alkyl and R^{2b} is H.
11. A compound of any preceding clause, or a pharmaceutically acceptable salt or N-oxide thereof, wherein R³ is C₁-C₄-alkyl.
12. A compound of any preceding clause, or a pharmaceutically acceptable salt or N-oxide thereof, wherein R⁴ is independently selected at each occurrence from C₁-C₄-alkyl, halo, cyano, C₁-C₄-haloalkyl, and C₀-C₄-alkylene-OR⁷.
13. A compound of any preceding clause, or a pharmaceutically acceptable salt or N-oxide thereof, wherein m is an integer selected from 0 or 1.
14. A pharmaceutical composition comprising a compound of any one of clauses 1 to 13, or a pharmaceutically acceptable salt or N-oxide thereof, and one or more pharmaceutically acceptable excipients.
15. A compound of any one of clauses 1 to 13, or a pharmaceutically acceptable salt or N-oxide thereof, for use as a medicament.
16. A compound of any one of clauses 1 to 13, or a pharmaceutically acceptable salt or N-oxide thereof, for use in treating a disease or disorder selected from an inflammatory disorder, an immune disorder, and an autoimmune disorder.
17. A compound of any one of clauses 1 to 13, or a pharmaceutically acceptable salt or N-oxide thereof, for use in treating a cancer.

### BRIEF DESCRIPTION OF THE DRAWINGS

FIGS. 1A-1E show plasma concentration of Example 101 in beagle dogs after intravenous or oral administration.

FIGS. 2A-2B depict body weight of Lewis rats over 21-day following induction of collagen-induced arthritis under various treatment conditions. Actual body weight (FIG. 2A) and percent change in body weight (FIG. 2B) are depicted.

FIGS. 3A-3C depict hind limb paw volume of Lewis rats over 21-day following induction of collagen-induced arthritis under various treatment conditions in left hind paw (FIG. 3A), right hind paw (FIG. 3B), and average of left and right hind paw volume (FIG. 3C).

FIGS. 4A-4B show clinical scoring of arthritis symptoms in Lewis rats over 21-day following induction of collagen-induced arthritis under various treatment conditions.

FIGS. 5A-5B show mean levels of rat anti-collagen IgG1 antibodies in animals at the end of the 21-day study. FIG. 5A also illustrates individual levels of rat anti-collagen IgG1 antibodies in animals at the end of the 21-day study.

FIGS. 6A-6C depict the pharmacokinetic profile of Example 101 in 3 individual Lewis rats after 10 mg/kg oral administration BID at 0 and 8 hours on Day 0 (FIG. 6A) and Day 20 (FIG. 6B) with an overlay of the average of these two studies depicted in FIG. 6C.

FIG. 7 depicts mean histopathological scores for tissue samples from animals in Groups 1-7. Animals in the vehicle group are represented by the solid white bar, animals in the CIA+Vehicle group are represented by the solid black bar, animals in the CIA+Dexamethasone group are represented by the grey bar with white checkers, animals in the CIA+GSK620 group are represented by the grey bar with black checkers, animals in the CIA+Example 101 (3 mg/kg) group are represented by the grey bar with the white diagonal slash, animals in the CIA+Example 101 (10 mg/kg) group are represented by the grey bar with the black diagonal slash, and animals in the CIA+Example 101 (30 mg/kg) group are represented by the grey bar with the brick pattern.

FIGS. 8A-8G depict representative tissue samples from animals in Groups 1-7 analyzed in the histopathological analysis.

FIGS. 9A-9B shows the study design for the CIA rat model (FIG. 9A) and UUO rat renal fibrosis model (FIG. 9B).

FIGS. 10A-10C depicts mean body weight percent change (FIG. 10A), mean histopathology score (including interstitial nephritis, collagen fiber deposition, and nephropathology) (FIG. 10B), and mean serum urea levels (FIG. 10C) of sham rats (solid bar in FIG. 10C), rats with UUO treated with vehicle (diagonally striped bar in FIGS. 10B-10C), and rats with UUO treated with Example 101 (30 mg/kg) (checkered bar in FIGS. 10B-10C).

FIGS. 11A-11B show mean fold change of mRNA levels of tissue biomarkers (Col1a1, TGF-b1, MCP-1, IL-1b, IL-6, IL-17, TNF-a, and Timp1) in sham rats (solid bar), rats with UUO treated with vehicle (diagonally striped bar), and rats with UUO treated with Example 101 (30 mg/kg) (checkered bar).

FIGS. 12A-12B depict individual hydroxyproline levels in tissue of sham rats, rats with UUO treated with vehicle, and rats with UUO treated with Example 101 (30 mg/kg). In FIG. 12A, mean hydroxyproline levels are depicted for sham rats (solid bar), rats with UUO treated with vehicle (diagonally striped bar), and rats with UUO treated with Compound A (10 mg/kg) (checkered bar).

FIGS. 13A-13B depict representative staining samples of tissue from rats with UUO treated with vehicle (FIG. 13A) and from rats with UUO treated with Example 101 (30 mg/kg) (FIG. 13B).

### DETAILED DESCRIPTION

As used herein, the term “about” has its usual meaning in the context of pharmaceutical and cosmetic formulations to allow for reasonable variations in amounts that can achieve the same effect, typically plus or minus up to 30%. For example, if an amount of “about 1” is provided, then the amount can be up to 1.3 or from 0.70. In cases where “about X” will lead to a figure of above 100%, the term in some embodiments can be read as reflecting up to 100% by weight less the total of the minimum amount of the other ingredients. Likewise, it will be appreciated by one skilled in the art to the extent X is reduced from that upper level the amounts of the other ingredients are increased appropriately. As will be appreciated by one of skill in the art, there is some reasonable flexibility in formulating compositions such that where one or more ingredients are varied, successful formulations can still be made even if an amount falls slightly outside the range. Therefore, to allow for this possibility, amounts are qualified by about. In some embodiments, the examples e.g., amounts of formulation ingredients can be read as if prefixed with the term “about.” In one or more other embodiments, the examples can be read without the term “about.” In some embodiments, the figures can be read with the term “about.” In one or more other embodiments, the figures can be read without the term “about.” In one or more narrower embodiments “about” can be plus or minus up to 15% unless the context indicates otherwise. Where “about” is used in connection with “>X” or “<X” or a series of such alternatives, it can in some embodiments, include about X. Where “about” is used just at the beginning of a series of alternative amounts of “>about X” or “<about X” or “about >X” or “about <X”, it can in some embodiments be understood to include “about” before all the other alternatives of the series.

The term Cₘ-Cₙ refers to a group with m to n carbon atoms. For the absence of doubt, the term “C₀” refers to a group with 0 carbon atoms.

The term “alkyl” refers to a monovalent linear or branched saturated hydrocarbon chain. For example, C₁-C₆-alkyl may refer to methyl, ethyl, n-propyl, iso-propyl, n-butyl, sec-butyl, tert-butyl, n-pentyl and n-hexyl. The alkyl groups may be unsubstituted or substituted by one or more substituents.

The term “alkylene” refers to a bivalent linear saturated hydrocarbon chain. For example, C₁-C₃-alkylene may refer to methylene, ethylene or propylene. The alkylene groups may be unsubstituted or substituted by one or more substituents. For the absence of doubt, the term “C₀-alkylene” refers to a group in which an alkylene chain is absent. For example, “C₀-alkylene-R^{a}” refers to an R^{a}.

The term “haloalkyl” refers to a hydrocarbon chain substituted with at least one halogen atom independently chosen at each occurrence from: fluorine, chlorine, bromine and iodine. The halogen atom may be present at any position on the hydrocarbon chain. For example, C₁-C₆-haloalkyl may refer to chloromethyl, fluoromethyl, trifluoromethyl, chloroethyl e.g., 1-chloromethyl and 2-chloroethyl, trichloroethyl e.g., 1,2,2-trichloroethyl, 2,2,2-trichloroethyl, fluoroethyl e.g., 1-fluoromethyl and 2-fluoroethyl, trifluoroethyl e.g., 1,2,2-trifluoroethyl and 2,2,2-trifluoroethyl, chloropropyl, trichloropropyl, fluoropropyl, trifluoropropyl.

A haloalkyl group may be a fluoroalkyl group, i.e., a hydrocarbon chain substituted with at least one fluorine atom. Thus, a haloalkyl group may have any amount of halogen substituents. The group may contain a single halogen substituent, it may have two or three halogen substituents, or it may be saturated with halogen substituents.

The term “alkenyl” refers to a branched or linear hydrocarbon chain containing at least one double bond. The double bond(s) may be present as the E or Z isomer. The double bond may be at any possible position of the hydrocarbon chain. For example, “C₂-C₆-alkenyl” may refer to ethenyl, propenyl, butenyl, butadienyl, pentenyl, pentadienyl, hexenyl and hexadienyl. The alkenyl groups may be unsubstituted or substituted by one or more substituents.

The term “alkynyl” refers to a branched or linear hydrocarbon chain containing at least one triple bond. The triple bond may be at any possible position of the hydrocarbon chain. For example, “C₂-C₆-alkynyl” may refer to ethynyl, propynyl, butynyl, pentynyl and hexynyl. The alkynyl groups may be unsubstituted or substituted by one or more substituents.

The term “cycloalkyl” refers to a saturated hydrocarbon ring system containing 3, 4, 5 or 6 carbon atoms. For example, “C₃-C₆-cycloalkyl” may refer to cyclopropyl, cyclobutyl, cyclopentyl, cyclohexyl. The cycloalkyl groups may be unsubstituted or substituted by one or more substituents. The cycloalkyl groups may be monocyclic or bicyclic. Bicyclic groups may be fused, spirofused or bridged.

The term “y- to z-membered heterocycloalkyl” refers to a monocyclic or bicyclic saturated or partially saturated group having from y to z atoms in the ring system and comprising 1 or 2 heteroatoms independently selected from O, S and N in the ring system (in other words 1 or 2 of the atoms forming the ring system are selected from O, S and N). By partially saturated it is meant that the ring may comprise one or two double bonds. This applies particularly to monocyclic rings with from 5 to 6 members. The double bond will typically be between two carbon atoms but may be between a carbon atom and a nitrogen atom. Examples of heterocycloalkyl groups include; oxirane, aziridine, thiirane, oxetane, azetidine, thietane, piperidine, piperazine, morpholine, thiomorpholine, pyrrolidine, tetrahydrofuran, tetrahydrothiophene, dihydrofuran, tetrahydropyran, dihydropyran, dioxane, and azepine. A heterocycloalkyl group may be unsubstituted or substituted by one or more substituents. The cycloalkyl groups may be monocyclic or bicyclic. Bicyclic groups may be fused, spirofused or bridged.

Aryl groups may be any aromatic carbocyclic ring system (i.e., a ring system containing 2(2n+1)π electrons). Aryl groups may have from 6 to 10 carbon atoms in the ring system. Aryl groups will typically be phenyl groups. Aryl groups may be naphthyl groups or biphenyl groups.

The term “heterocyclyl” groups refers to rings comprising from 1 to 4 heteroatoms independently selected from O, S and N. The rings may be heterocycloalkyl rings (including both saturated and partially saturated rings) or heteroaryl rings. The term “heterocyclyl” also encompasses groups that are tautomers of hydroxy heteroaryl groups, such pyridones, and tautomers of hydroxy heteroaryl groups that are substituted on the nitrogen, such as N-alkyl pyridones. The term “heterocyclyl” includes a saturated, unsaturated or aromatic ring system containing at least one heteroatom selected from N, O or S. A “heterocyclic” system may contain 1, 2, 3 or 4 heteroatoms, for example 1 or 2. A “heterocyclic” system may be monocyclic or a fused polycyclic ring system, for example, bicyclic or tricyclic. A “heterocyclic” moiety may contain from 3 to 14 carbon atoms, for example, 3 to 8 carbon atoms in a monocyclic system and 7 to 14 carbon atoms in a polycyclic system. “Heterocyclic” encompasses heterocycloalkyl moieties, heterocycloalkenyl moieties and heteroaryl moieties. “Heterocyclic” also encompasses bicyclic groups in which one ring is phenyl and the other ring is a heterocycloalkyl or heterocycloalkenyl ring. “Heterocyclic” also encompasses bicyclic groups in which one ring is heteroaryl and the other ring is a cycloalkyl or cycloalkenyl ring.

For example, the heterocyclic group may be: oxirane, aziridine, azetidine, oxetane, tetrahydrofuran, pyrrolidine, imidazolidine, succinimide, pyrazolidine, oxazolidine, isoxazolidine, thiazolidine, isothiazolidine, piperidine, morpholine, thiomorpholine, piperazine, and tetrahydropyran. Heteroaryl includes groups such as pyridones and N-alkyl-pyridones.

The term “heterocycloalkenyl” refers to partially saturated rings comprising from 1 to 2 heteroatoms independently selected from O, S and N.

The term “heteroaryl” refers to any aromatic (i.e., a ring system containing 2(2n+1)π electrons) 5 or 6 membered ring system comprising from 1 to 4 heteroatoms independently selected from O, S and N (in other words from 1 to 4 of the atoms forming the ring system are selected from O, S and N). Thus, any heteroaryl groups may be independently selected from: 5 membered heteroaryl groups in which the heteroaromatic ring is substituted with 1-4 heteroatoms independently selected from O, S and N; and 6-membered heteroaryl groups in which the heteroaromatic ring is substituted with 1-3 (e.g., 1-2) nitrogen atoms. Specifically, heteroaryl groups may be independently selected from: pyrrole, furan, thiophene, pyrazole, imidazole, oxazole, isoxazole, triazole, oxadiazole, thiadiazole, tetrazole; pyridine, pyridazine, pyrimidine, pyrazine, triazine.

For variables which may be selected from “carbon” and “nitrogen” (i.e., X¹, X², X³, X⁴, X⁵, etc.) it is understood that the carbon or nitrogen may additionally comprise hydrogen and/or a designated substituent to the ring system (i.e., —R^{2a}, R⁴).

On ring systems designating an optional substituent (i.e., —R^{2a}, R⁴), it is understood that the substituent, if present, may replace a hydrogen on any carbon or nitrogen of the ring system.

Compounds disclosed herein containing one or more asymmetric carbon atoms can exist as two or more stereoisomers. Certain compounds of the disclosure may exist in particular geometric and/or stereoisomeric forms and the present disclosure contemplates all such compounds, including cis- and trans-isomers, R- and S-enantiomers, diastereomers, the racemic mixtures thereof, and other mixtures thereof, as falling within the scope of the disclosure. Additional asymmetric carbon atoms may be present in a substituent such as an alkyl group. All such isomers, as well as mixtures thereof, are included in this disclosure.

Where a compound disclosed herein contains a double bond such as a C═C or C═N group, geometric cis/trans (or Z/E) isomers are possible. Where structural isomers are interconvertible via a low energy barrier, tautomeric isomerism (‘tautomerism’) can occur. This can take the form of proton tautomerism in compounds disclosed herein containing, for example, an imino, keto, or oxime group, or so-called valence tautomerism in compounds which contain an aromatic moiety. It follows that a single compound may exhibit more than one type of isomerism.

Included within the scope of the present disclosure are all stereoisomers, geometric isomers and tautomeric forms of the compounds disclosed herein, including compounds exhibiting more than one type of isomerism, and mixtures of one or more thereof. Also included are acid addition or base salts wherein the counter ion is optically active, for example, d-lactate or I-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high pressure liquid chromatography (HPLC). Thus, chiral compounds of the disclosure (and chiral precursors thereof) may be obtained in enantiomerically-enriched form using chromatography, typically HPLC, on an asymmetric resin with a mobile phase consisting of a hydrocarbon, typically heptane or hexane, containing from about 0 to about 50% by volume of isopropanol, typically from about 2% to about 20%, and for specific examples, about 0 to about 5% by volume of an alkylamine e.g., about 0.1% diethylamine. Concentration of the eluate affords the enriched mixture.

The compound may substantially be the faster eluting enantiomer in chiral HPLC performed using any one of the columns outlined herein under Purification Methods. In particular, the compound may substantially be the faster eluting enantiomer prepared as Example 97 or 101 in the Examples below. The compound may substantially be the slower eluting enantiomer in chiral HPLC performed using any one of the columns outlined herein under Purification Methods. The column may comprise immobilised cellulose tris(4-methylbenzoate) (e.g., CHIRAL ART Cellulose-SJ) or cellulose tris(3,5-dimethylphenylcarbamate) (e.g., CHIRAL ART Cellulose SB). The solvent system used as the mobile phase in the HPLC may be any of those described below under Purification Methods.

Alternatively, the racemate (or a racemic precursor) may be reacted with a suitable optically active compound, for example, an alcohol, or, in the case where the compound disclosed herein contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

When needing to identify which enantiomer of a compound is present in a highly enantiopure sample, retention time on a chiral column may sometime be used. It will be appreciated, however, that such retention times may vary according to the particular chromatography conditions utilised. For example, the skilled person will appreciate that the temperature at which the separation is performed and/or the age and condition of the column used may impact retention time. In some circumstances, therefore, it is preferable to identify which enantiomer of a compound is by making one of the two enantiomers via a known method, e.g., the methods described in the Examples of this specification, and comparing the relative retention times on a chiral column of the resultant compound with the sample.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts.

The present disclosure also includes all pharmaceutically acceptable isotopically-labelled compounds of formula (I), wherein one or more atoms are replaced by atoms having the same atomic number, but an atomic mass or mass number different from the atomic mass or mass number usually found in nature.

Examples of isotopes suitable for inclusion in the compounds of the disclosure include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Isotopically-labelled compounds can generally be prepared by conventional techniques known to those skilled in the art or by processes analogous to those described using an appropriate isotopically-labelled reagent in place of the non-labelled reagent previously employed.

The activity of the compounds of the present disclosure can be assessed by a variety of in silico, in vitro and in vivo assays. In silico analysis of a variety of compounds has been demonstrated to be predictive of ultimate in vitro and even in vivo activity.

As used herein, the term “appendage” includes a hand, a foot, a wrist, an ankle, and/or a joint.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” includes the amount of a compound that, when administered to a mammal for treating a disease, is sufficient to affect such treatment for the disease. The “therapeutically effective amount” will vary depending on the compound, the disease and its severity and the age, weight, etc., of the mammal to be treated.

A compound disclosed herein, or pharmaceutically acceptable salt thereof, may be used on their own but will generally be administered in the form of a pharmaceutical composition in which the compounds disclosed herein, or pharmaceutically acceptable salt thereof, is in association with a pharmaceutically acceptable adjuvant, diluent or carrier.

Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration of the compounds disclosed herein, the pharmaceutical composition which is used to administer the compounds disclosed herein will in some embodiments comprise from about 0.005 to about 99% w/w compounds disclosed herein, or comprise from about 0.05 to about 80% w/w compounds disclosed herein, or comprise from about 0.10 to about 70% w/w compounds disclosed herein, or comprise from about 0.10 to about 50% w/w compounds disclosed herein (all percentages by weight being based on total composition). In some embodiments the pharmaceutical composition which is used to administer the compounds disclosed herein will comprise from about 0.005 to about 40% w/w compounds disclosed herein, or comprise from about 0.005 to about 30% w/w compounds disclosed herein, or comprise from about 0.010 to about 20% w/w compounds disclosed herein, or comprise from about 0.010 to about 10% w/w compounds disclosed herein or comprise from about 0.005 to about 5% w/w compounds disclosed herein, or comprise from about 0.005 to about 2% w/w compounds disclosed herein, or comprise from about 0.005 to about 1% w/w compounds disclosed herein, or comprise from about 0.005 to about 0.5% w/w compounds disclosed herein, or comprise from about 0.010 to about 1% w/w compounds disclosed herein, or comprise from about 0.010 to about 0.5% w/w compounds disclosed herein (all percentages by weight being based on total composition).

The pharmaceutical compositions may be administered topically (e.g., to the skin) in the form, e.g., of creams, ointments, gels, lotions, solutions, suspensions; or systemically, e.g., by oral administration in the form of tablets, lozenges, hard or soft capsules, solutions, aqueous or oily suspensions, emulsions, dispersible powders or granules, syrups or elixirs; or by parenteral administration in the form of a sterile aqueous or oily solution, suspension or emulsion for injection (including intraarticular, intravenous, intracoronary, subcutaneous, intramyocardial, intraperitoneal, intramuscular, intravascular or infusion); by rectal administration in the form of suppositories or enemas; by inhalation for example as a finely divided powder or a liquid aerosol or mist; or for administration by insufflation (for example as a finely divided powder).

For oral administration the compounds disclosed herein may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent. Thus, compositions intended for oral use may contain, for example, one or more colouring, sweetening, flavouring and/or preservative agents.

For the preparation of soft gelatine capsules, the compounds disclosed herein may be admixed with, for example, a vegetable oil or polyethylene glycol. Hard gelatine capsules may contain granules of the compound using either of the above-mentioned excipients for tablets. Also, liquid or semisolid formulations of the compound disclosed herein may be filled into hard gelatine capsules. Liquid preparations for oral application may be in the form of syrups or suspensions, for example, solutions containing the compound disclosed herein, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in art.

For intravenous (parenteral) administration the compounds disclosed herein may be administered as a sterile aqueous or oily solution.

The size of the dose for therapeutic or prophylactic purposes of a compound disclosed herein will naturally vary according to the nature and severity of the conditions, the concentration of the compound required for effectiveness in isolated cells, the concentration of the compound required for effectiveness in experimental animals, the age and sex of the animal or patient and the route of administration, according to well known principles of medicine.

Dosage levels, dose frequency, and treatment durations of compounds disclosed herein are expected to differ depending on the formulation and clinical indication, age, and co-morbid medical conditions of the patient.

An effective amount of a compound of the present disclosure for use in therapy of a condition is an amount sufficient to achieve symptomatic relief in a warm-blooded animal, particularly a human of the symptoms of the condition, to mitigate the physical manifestations of the condition, or to slow the progression of the condition.

The amount of active ingredient that is combined with one or more excipients to produce a single dosage form will necessarily vary depending upon the host treated and the particular route of administration. For example, a formulation intended for oral administration to humans will generally contain, for example, from about 0.5 mg to about 0.5 g of active agent (more suitably from about 0.5 to about 100 mg, for example from about 1 to about 30 mg) compounded with an appropriate and convenient amount of excipients which may vary from about 5 to about 98 or about 99 percent by weight of the total composition.

For the above-mentioned compounds disclosed herein the dosage administered will, of course, vary with the compound employed, the mode of administration, the treatment desired and the disorder indicated. In using a compound disclosed herein for therapeutic or prophylactic purposes it will generally be administered so that a daily dose in the range, for example, a daily dose selected from about 0.1 mg/kg to about 100 mg/kg, about 1 mg/kg to about 75 mg/kg, about 1 mg/kg to about 50 mg/kg, about 1 mg/kg to about 20 mg/kg or about 5 mg/kg to about 10 mg/kg body weight is received, given if required in divided doses. In general, lower doses will be administered when a parenteral route is employed. Thus, for example, for intravenous or intraperitoneal administration, a dose in the range, for example, about 0.1 mg/kg to about 30 mg/kg body weight will generally be used. Similarly, for administration by intraarticular, a dose in the range, for example, about 0.01 mg/kg to about 30 mg/kg body weight may generally be used. For administration by inhalation, a dose in the range, for example, about 0.05 mg/kg to about 25 mg/kg body weight may be used. Suitably the compound disclosed herein is administered orally, for example in the form of a tablet, or capsule dosage form. The daily dose administered orally may be, for example a total daily dose selected from about 1 mg to about 1000 mg, about 5 mg to about 1000 mg, about 10 mg to about 750 mg or about 25 mg to about 500 mg. Typically, unit dosage forms will contain about 0.5 mg to about 0.5 g of a compound of this disclosure.

The compounds disclosed herein may be administered along with other active compounds as part of a treatment regime. The other active compounds may be administered simultaneously with, subsequently to or previously to the administration of the compounds disclosed herein. It may be that the pharmaceutical formulation comprising the compounds disclosed herein also comprises one or more other active compounds. The other active compounds may be anticancer, anti-inflammatory, antibacterial, antiviral, antiemetic, antithrombotic or compounds that alter the metabolism.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. Where a point is provided, or a data point was determined, that data point can be considered in one embodiment as a single data point and in another embodiment as a mean of two or more data points. Similarly, where a range between two points is provided, each one of those two data points can be considered in one embodiment as a single data point and in another embodiment as a mean of two or more data points. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example disclosed herein are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

It will be understood that various modifications may be made to the embodiments disclosed herein. Therefore, the above description should not be construed as limiting, but merely as exemplifications of embodiments. For example, the functions described above and implemented as the best mode for operating the present disclosure are for illustration purposes only. Other arrangements and methods may be implemented by those skilled in the art without departing from the scope and spirit of this disclosure. Moreover, those skilled in the art will envision other modifications within the scope and spirit of the specification appended hereto.

The reader's attention is directed to all papers and documents which are filed concurrently with or previous to this specification in connection with this application and which are open to public inspection with this specification, and the contents of all such papers and documents are incorporated herein by reference.

In some embodiments, the present disclosure is directed to methods of using a compound of formula (I), or a tautomer, a stereoisomer or a mixture of stereoisomers, a pharmaceutically acceptable salt, a hydrate, a deuterated derivative, or an N-oxide thereof:

[symbol]

wherein Ring A, X, Z, R^{1a}, R^{1b}, R², R³, R⁴ and m are as herein described.

In some embodiments, the compound of formula (I) is a compound of formula (IIA), or a tautomer, a stereoisomer or a mixture of stereoisomers, a pharmaceutically acceptable salt, a hydrate, a deuterated derivative, or an N-oxide thereof:

[symbol]

wherein Ring A, X, Z, R^{1a}, R^{1b}, R², R³, R⁴ and m are as herein described.

In some embodiments, the compound of formula (I) is a compound of formula (IIB), or a tautomer, a stereoisomer or a mixture of stereoisomers, a pharmaceutically acceptable salt, a hydrate, a deuterated derivative, or an N-oxide thereof:

[symbol]

wherein Ring A, X, Z, R^{1a}, R^{1b}, R², R³, R⁴ and m are as herein described.

In some embodiments, the compound of formula (I) is a compound of formula (XIIIA), or a tautomer, a stereoisomer or a mixture of stereoisomers, a pharmaceutically acceptable salt, a hydrate, a deuterated derivative, or an N-oxide thereof:

[symbol]

wherein R^{1a}, R^{2a}, R^{2b}, R³, R⁴, R¹⁰ and m are as herein described.

In some embodiments, the compound of formula (I) is a compound of formula (XIIIB), or a tautomer, a stereoisomer or a mixture of stereoisomers, a pharmaceutically acceptable salt, a hydrate, a deuterated derivative, or an N-oxide thereof:

[symbol]

wherein R^{1a}, R^{2a}, R^{2b}, R³, R⁴, R¹⁰ and m are as herein described.

In some embodiments, the compound of formula (I) is selected from Examples 1-128 as disclosed herein, or a tautomer, a stereoisomer or a mixture of stereoisomers, a pharmaceutically acceptable salt, a hydrate, a deuterated derivative, or an N-oxide thereof.

In some embodiments, the present disclosure provides methods of using a pharmaceutical composition comprising a compound disclosed herein and one or more pharmaceutically acceptable excipients.

In some embodiments, the present disclosure provides a compound or pharmaceutical composition of any of the embodiments disclosed herein for use in a method of treatment of an immuno or autoimmune disease, e.g., arthritis, rheumatoid arthritis, psoriasis/psoriatic arthritis, and/or an inflammatory disease, e.g., inflammatory skin disorders, respiratory diseases, gastrointestinal diseases, eye diseases, cancers, rheumatic diseases, demyelinating diseases, fibrotic diseases, and/or a myeloproliferative neoplastic disorder.

In one or more embodiments, the present disclosure provides a compound or pharmaceutical composition of any of the embodiments disclosed herein, for the treatment of an immuno or autoimmune disease, e.g., arthritis, rheumatoid arthritis, psoriasis/psoriatic arthritis, and/or an inflammatory disease, e.g., inflammatory skin disorders, respiratory diseases, gastrointestinal diseases, eye diseases, cancers, rheumatic diseases, demyelinating diseases, fibrotic diseases, and/or a myeloproliferative neoplastic disorder, wherein said treatment comprises administering to a subject, an effective amount of a compound or a pharmaceutical composition disclosed herein.

In some embodiments, the present disclosure provides a compound or pharmaceutical composition of any of the embodiments disclosed herein, for the manufacture of a medicament for the treatment of an immuno or autoimmune disease, e.g., arthritis, rheumatoid arthritis, psoriasis/psoriatic arthritis, and/or an inflammatory disease, e.g., inflammatory skin disorders, respiratory diseases, gastrointestinal diseases, eye diseases, cancers, rheumatic diseases, demyelinating diseases, fibrotic diseases, and/or a myeloproliferative neoplastic disorder, wherein said treatment comprises administering to a subject an effective amount of the compound or pharmaceutical composition disclosed herein.

In some embodiments, the present disclosure provides a compound or pharmaceutical composition of any of the embodiments disclosed herein, for use in a method of inhibiting Bromodomain and Extra-Terminal protein activity in a subject comprising administering to a subject an effective amount of the compound or pharmaceutical composition disclosed herein.

In some embodiments, the present disclosure provides a compound or pharmaceutical composition of any of the embodiments disclosed herein, for use in a method of treating a disorder associated with Bromodomain and Extra-Terminal protein activity in a subject, said method comprising administering to a subject an effective amount of the compound disclosed herein, or a pharmaceutical composition disclosed herein.

In some embodiments, the BET BDII protein inhibitors disclosed herein are active and selective for BDII over BD1. In some embodiments, BET BDII selective protein inhibitors disclosed herein exhibit greater than about 200-Fold selectivity. In some embodiments, BET BDII selective protein inhibitors disclosed herein exhibit greater than about 300-Fold, greater than about 400-Fold, greater than about 500-Fold, or greater than about 600-Fold, selectivity for BDII over BD1. In some embodiments, BET BDII selective protein inhibitors disclosed herein exhibit greater than about 1000-Fold selectivity for BDII over BD1. In some embodiments, they exhibit greater than about 2000-Fold selectivity. In some embodiments, they exhibit greater than about 5000-Fold selectivity for BDII over BD1. In some embodiments the selectivity is between about 200 to about 10,000, or between about 200 to about 6000, or between about 200 and about 1000. In one or more embodiments, BET BDII selective protein inhibitors disclosed herein exhibit an IC50 of <about 250 nM, <about 200 nM, <about 150 nM, <about 100 nM, <about 75 nM, <about 50 nM or <about 25 nM for BRD4 BDII. In some embodiments, the IC50 is <about 20 nM or <about 15 nM for BRD4 BDII. In one or more embodiments, BET BDII selective protein inhibitors disclosed herein exhibit an IC50 ranging from <about 200 nM to <about 10 nM for BRD4 BDII. In some embodiments, BET BDII selective protein inhibitors disclosed herein exhibit an IC50 ranging from about 50 nM to about 5 nM for BRD4 BDII. In some embodiments, the IC50 is a mean value of two or more measurements.

In some embodiments one or more compounds described herein are more selective for IL-22 than GSK620. In some embodiments one or more compounds described herein are two-fold, three-fold, four-fold or five-fold more selective for IL-22 than GSK620. In some embodiments one or more compounds described herein are ten-fold or twenty-fold more selective for IL-22 than GSK620. In some embodiments one or more compounds described herein are more selective for IL-17A than GSK620. In some embodiments one or more compounds described herein are two-fold, or three-fold more selective for IL-17A than GSK620.

A BET-inhibiting compound, such as the compounds disclosed herein, may in one or more embodiments, be of value and used in the treatment of the following non-limiting examples of disorders and diseases.

A BET-inhibiting compound, such as the compounds disclosed herein, may in one or more embodiments, be of value and used in the treatment of inflammatory disorders, immune disorders, and autoimmune disorders, which include diseases that have or may have an inflammatory or autoimmune component.

In some embodiments, the present disclosure provides a compound or a pharmaceutical composition as defined in this disclosure for use in a method of treatment of an inflammatory disease, e.g., inflammatory skin disorders, respiratory diseases, gastrointestinal diseases, eye diseases, cancers, rheumatic diseases, demyelinating diseases and fibrotic diseases and/or an immuno or autoimmune disease, e.g., arthritis, rheumatoid arthritis, psoriasis/psoriatic arthritis, multiple sclerosis, lupus, systemic lupus erythematosus, inflammatory bowel disease, Addison's disease, Graves' disease, Sjögren's syndrome, thyroiditis, myasthenia gravis, Guillain-Barre syndrome, chronic inflammatory demyelinating polyneuropathy, scleroderma and autoimmune vasculitis, and or a myeloproliferative neoplastic disorder, e.g., chronic myelogenous leukemia, polycythemia vera, primary myelofibrosis (also called chronic idiopathic myelofibrosis), essential thrombocythemia, chronic neutrophilic leukemia, chronic eosinophilic leukemia, and acute leukemia. Immuno-inflammatory indications include rheumatoid arthritis, systemic lupus erythematosus, ulcerative colitis/Crohn's and multiple sclerosis.

In some embodiments, the present disclosure provides a method for the treatment of an inflammatory disease, e.g., inflammatory skin disorders, respiratory diseases, gastrointestinal diseases, eye diseases, cancers, rheumatic diseases, demyelinating diseases, and fibrotic diseases, and/or an immuno or autoimmune disease, e.g., arthritis, rheumatoid arthritis, psoriasis/psoriatic arthritis, multiple sclerosis, lupus, systemic lupus erythematosus, inflammatory bowel disease, Addison's disease, Graves' disease, Sjögren's syndrome, thyroiditis, myasthenia gravis, Guillain-Barre syndrome, chronic inflammatory demyelinating polyneuropathy, scleroderma and autoimmune vasculitis, and or a myeloproliferative neoplastic disorder, e.g., chronic myelogenous leukemia, polycythemia vera, primary myelofibrosis (also called chronic idiopathic myelofibrosis), essential thrombocythemia, chronic neutrophilic leukemia, chronic eosinophilic leukemia, and acute leukemia. Immuno-inflammatory indications include rheumatoid arthritis, systemic lupus erythematosus, ulcerative colitis/Crohn's and multiple sclerosis. said method comprising administering to a subject, an effective amount of a compound or a pharmaceutical composition as defined in this disclosure.

In some embodiments, the present disclosure provides the use of a compound or a pharmaceutical composition as defined in this disclosure for the manufacture of a medicament for the treatment of an inflammatory disease, e.g., inflammatory skin disorders, respiratory diseases, gastrointestinal diseases, eye diseases, cancers, rheumatic diseases, demyelinating diseases, and fibrotic diseases and/or an immuno or autoimmune disease, e.g., arthritis, rheumatoid arthritis, psoriasis/psoriatic arthritis, multiple sclerosis, lupus, systemic lupus erythematosus, inflammatory bowel disease, Addison's disease, Graves' disease, Sjögren's syndrome, thyroiditis, myasthenia gravis, Guillain-Barre syndrome, chronic inflammatory demyelinating polyneuropathy, scleroderma and autoimmune vasculitis, and or a myeloproliferative neoplastic disorder, e.g., chronic myelogenous leukemia, polycythemia vera, primary myelofibrosis (also called chronic idiopathic myelofibrosis), essential thrombocythemia, chronic neutrophilic leukemia, chronic eosinophilic leukemia, and acute leukemia. Immuno-inflammatory indications include rheumatoid arthritis, systemic lupus erythematosus, ulcerative colitis/Crohn's and multiple sclerosis, said method comprising administering to a subject, an effective amount of a compound or a pharmaceutical composition as defined in this disclosure.

The present disclosure provides BET inhibitors (e.g., Example 101) that can provide a new and effective treatment and relief for diseases with an inflammatory and/or autoimmune component, such as joint related diseases and disorders. Joints may be infected by many types of microorganisms (bacteria, fungi, viruses) and occasionally by animal parasites. Infection related joint diseases and disorders include infection by direct contamination, by way of the bloodstream e.g., through the synovial blood vessels, and by extension from adjacent bony infections (osteomyelitis). Infectious arthritis may affect one joint (monarthritis) or a few joints (oligoarthritis) rather than many (polyarthritis). Joints or parts thereof can be damaged e.g., cartilage by for example through staphylococci, hemolytic streptococci, and pneumococci infections, e.g., bone through tuberculosis such as tuberculous spondylitis (Pott disease), or through Coccidioides immitis, brucellosis, such as Brucella suis, leprosy (Hansen disease), rubella (German measles) and serum hepatitis, viral synovitis, dranunculiasis (Guinea worm disease), sexually transmitted diseases, including gonorrhea, reactive arthritis (Reiter disease), congenital syphilis such as Clutton joint lesion, and Yaws, which leads to skeletal lesions. Inflammation may destroy the joint cartilage and underlying bone and cause irreparable deformities. Adhesions between the articulating members are frequent in such cases, and the resulting fusion with loss of mobility is called ankylosis such as ankylosing spondylitis, (Marie-Strümpell disease or Bechterew disease). Another type of arthritis is associated with chronic intestinal diseases—ulcerative colitis, regional enteritis, inflammatory bowel disease, cirrhosis, and Whipple disease. In addition to joint disorders and diseases resulting from any of the above the present disclosure provides a BET inhibitor (Example 101) that may also provide a new and effective treatment or relief for noninflammatory joint diseases, injury and degenerative disorders. Trauma to joints includes blunt injuries, mild sprains, fractures and dislocations, ligamentous, tendinous, and capsular tears, tears in the semilunar cartilages (menisci), and hemarthrosis. Degenerative joint disease includes osteoarthritis, arthrosis deformans, precocious osteoarthritis congenital dysplasia malum coxae senilis, spondylosis, chrondromalacia patellae, metabolic diseases such gouty arthritis, podagra, ochronotic arthropathy, chondrocalcinosis, or pseudogout, mucopolysaccharidoses, Hurler syndrome, Morquio disease, and polyepiphyseal dysplasias.

The present disclosure also provides BET inhibitors (e.g., Example 101) that may provide a new and effective treatment or relief for secondary joint diseases and disorders, including hemorrhagic joints, hemarthrosis, villonodular synovitis, joint diseases that arise in association with aseptic necrosis e.g., can occur with fractures, osteochondritis dissecans, slipped epiphysis, Osgood-Schlatter, Legg-Calve-Perthes, endocrine-malfunctioning resultant joint disorders, acromegaly, neurogenic arthropathy, Charcot joint, hypertrophic osteoarthropathy, reflex sympathetic dystrophy, joint tumors, synovial chondromatosis, cartilaginous nodules, synovial osteochondromatosism, synoviomas, synovial sarcomas, and polymyalgia rheumatica.

The present disclosure also provides BET inhibitors (e.g., Example 101) that may provide a new and effective treatment or relief for fibrosis or fibrosis-associated conditions affecting any tissue including, for example, fibrosis of an internal organ, a cutaneous or dermal fibrosing disorder, and fibrotic conditions of the eye. In some embodiments, the fibrosis or fibrosis-associated conditions include fibrosis of internal organs (e.g., liver, lung, kidney, heart blood vessels, gastrointestinal tract). In some embodiments, the fibrosis or fibrosis-associated conditions include pulmonary fibrosis, idiopathic fibrosis, autoimmune fibrosis, myelofibrosis, liver cirrhosis, veno-occlusive disease, mesangial proliferative glomerulonephritis, crescentic glomerulonephritis, diabetic nephropathy, renal interstitial fibrosis, renal fibrosis in subjects receiving cyclosporin, allograft rejection, HIV associated nephropathy. In some embodiments, the fibrosis-associated disorders include systemic sclerosis, eosinophilia-myalgia syndrome, and fibrosis-associated CNS disorders such as intraocular fibrosis. In some embodiments, dermal fibrosis disorders include, for example, scleroderma, morphea, keloids, hypertrophic scars, familial cutaneous collagenoma, and connective tissue nevi of the collagen type. In some embodiments, fibrotic conditions of the eye include conditions such as diabetic retinopathy, post-surgical scarring (for example, after glaucoma filtering surgery and after crossed-eyes (strabismus) surgery), and proliferative vitreoretinopathy. In some embodiments, fibrotic conditions that may be treated by the methods of the present invention may result, for example, from rheumatoid arthritis, diseases associated with prolonged joint pain and deteriorated joints, progressive systemic sclerosis, polymyositis, dermatomyositis, eosinophilic fascitis, morphea, Raynaud's syndrome, and nasal polyposis.

In some embodiments, there is provided one or more compounds and compounds that can function as a selective BD BET inhibitor. In some embodiments, the one or more compounds that can impact positively a joint or joint related disease and disorder and/or a fibrosis or fibrosis related disease and disorder involving multiple, diverse inflammatory cell signaling pathways. In some embodiments, there is provided one or more compounds that are applicable to and can have therapeutic activity, to specific joint or joint related diseases and disorders and/or fibrosis or fibrosis related diseases and disorders. In some embodiments, there is provided at least one compound applicable to and having therapeutic activity to specific joint or joint related secondary diseases and disorders. In some embodiments, the disclosed compounds and compositions reduce inflammation in the joint and or in the surrounding tissues. In some embodiments, the joint or joint related diseases and disorders are chosen from arthritis, bursitis, Ehlers-Danlos syndrome, epicondylitis, Felty Syndrome, Sgouty arthritis, psoriatic arthritis, osteoarthritis, rheumatoid arthritis, Sill's disease, tenosynovitis, synovitis, Sjögren's Syndrome, Lyme disease, Whipple disease, bone cancer, lupus, and other autoimmune joint disorders. In some embodiments, the disease is rheumatoid arthritis.

In some embodiments, the present disclosure provides BET inhibitors that can provide a new and effective treatment and relief for fibrotic diseases or fibrosis (e.g., kidney or renal fibrosis). For example, in some embodiments, the present disclosure provides specific BET inhibitors that can retard the progression or severity of indicators of fibrosis, e.g., kidney fibrosis.

The methods and compositions of the present disclosure can, in some embodiments, be useful therapeutically for a fibrosis or fibrosis-associated conditions affecting any tissue including, for example, fibrosis of an internal organ, a cutaneous or dermal fibrosing disorder, and fibrotic conditions of the eye. In some embodiments, the fibrosis or fibrosis-associated conditions include fibrosis of internal organs (e.g., liver, lung, kidney, heart blood vessels, gastrointestinal tract). In some embodiments, the fibrosis or fibrosis-associated conditions include pulmonary fibrosis, idiopathic fibrosis, autoimmune fibrosis, myelofibrosis, liver cirrhosis, veno-occlusive disease, mesangial proliferative glomerulonephritis, crescentic glomerulonephritis, diabetic nephropathy, renal interstitial fibrosis, renal fibrosis in subjects receiving cyclosporin, allograft rejection, HIV associated nephropathy. In some embodiments, the fibrosis-associated disorders include systemic sclerosis, eosinophilia-myalgia syndrome, and fibrosis-associated CNS disorders such as intraocular fibrosis. In some embodiments, dermal fibrosis disorders include, for example, scleroderma, morphea, keloids, hypertrophic scars, familial cutaneous collagenoma, and connective tissue nevi of the collagen type. In some embodiments, fibrotic conditions of the eye include conditions such as diabetic retinopathy, post-surgical scarring (for example, after glaucoma filtering surgery and after crossed-eyes (strabismus) surgery), and proliferative vitreoretinopathy. In some embodiments, fibrotic conditions that may be treated by the methods of the present invention may result, for example, from rheumatoid arthritis, diseases associated with prolonged joint pain and deteriorated joints, progressive systemic sclerosis, polymyositis, dermatomyositis, eosinophilic fascitis, morphea, Raynaud's syndrome, and nasal polyposis.

In some embodiments, the present disclosure provides specific BET inhibitors that have been found to be surprisingly effective against renal fibrosis and renal fibrosis-related conditions and/or may provide a suitable treatment in limiting or slowing its progression. In some embodiments, the present disclosure provides potent and selective BET inhibitors that can provide a new and effective treatment and relief for fibrosis and fibrosis-related conditions, e.g., renal fibrosis and renal fibrosis-related conditions and/or limit or slow its progression. In some embodiments, the present disclosure provides potent and selective BET inhibitors that can provide new and effective treatment or relief for inflammatory fibrosis (e.g., renal fibrosis) and/or limit or slow its progression.

In some embodiments, the present disclosure provides potent and selective BET inhibitors (e.g., compounds of formula (I)) that may also provide new and effective treatment or relief for noninflammatory fibrosis (e.g., renal fibrosis) diseases, injury, and degenerative disorders and/or limit or slow their progression.

In some embodiments, use of the compounds to treat a disease as disclosed herein results in a therapeutic effect associated with a reduction in disease. In some embodiments, use of the compounds to treat a disease or disorder as disclosed herein results in a reduction of one or more tissue inflammation biomarkers selected from Col1A, TGF-b1, MCP-1, IL-1b, IL-6, IL-17, TNF-a, and Timp1. In some embodiments, use of the compounds to treat a disease or disorder as disclosed herein results in a reduction of one or more tissue inflammation biomarkers selected from Col1A, TGF-b1, MCP-1, IL-1b, IL-6, and Timp1. In some embodiments, use of the compounds to treat a disease or disorder as herein disclosed results in IL-17 and/or TNF-a being relatively unchanged. In some embodiments, use of the compounds to treat a disease or disorder as herein disclosed results in a small reduction in IL-17 and/or TNF-a,

Treatment with a BET-inhibiting compound, such as compositions comprising the compounds disclosed herein or salts thereof (or combinations thereof), in some embodiments may be effective if applied orally, in some other embodiments may be effective if applied by injection, in some other embodiments may be effective if applied topically, and in some further embodiments may be effective if applied topically and orally or by injection and topically or by orally and injection. In some embodiments, treatment with a BET-inhibiting compound, such as compositions comprising the compounds disclosed herein or salts thereof (or combinations thereof), may be effective orally where the compounds have a useful, e.g., >about 20% or good bioavailability e.g., >about 25%.

In some embodiments, one or more compounds disclosed herein are applied orally, for example as a solid dose form e.g., as a tablet, or a capsule, or as a semisolid or fluid dose form e.g., as a gel, or as liquid. In a fluid or semisolid dosage form the compound may in one or more embodiments be delivered as a suspension or as a solution.

In some embodiments, one or more compounds disclosed herein are applied by injection, e.g., as a solution or as a suspension. The solution or suspension may be in one or more embodiments, e.g., aqueous based, oil based, waterless, hydrophilic, hydrophobic, amphiphilic and or an emulsion.

In some embodiments, one or more compounds disclosed herein are applied by inhalation, e.g., as a powder, spray or mist. In a fluid or liquid form, which can be used to form a mist (e.g., with a nebulizer) or spray (e.g., with an aerosol) the compound may in one or more embodiments be delivered as a suspension or as a solution.

In some embodiments, one or more compounds disclosed herein are applied topically e.g., as a cream, emulsion, lotion, gel, ointment, mousse, foam, spray or other topical dosage formats known in the art. In some embodiments, when applied topically, the compounds disclosed herein may be effective where the compound is delivered primarily or substantially into the skin with low levels of transdermal penetration. In some embodiments, when applied topically the compounds disclosed herein may be effective where the compound is delivered primarily or substantially transdermally. In some embodiments, when applied topically the compounds disclosed herein may be effective where the compound is delivered intradermally and transdermally. In some embodiments, the penetration of the compound in the epidermis can be higher than that in the dermis. In some embodiments, the penetration of the compound in the dermis can be higher than in the epidermis. In some embodiments the penetration of the compound in the dermis is similar to that in the epidermis. In some embodiments, the concentration of the compound per unit volume in the epidermis can be higher than that in the dermis. In some embodiments, the concentration of the compound per unit volume in the dermis can be higher than in the epidermis. In some embodiments, the concentration of the compound per unit volume in the dermis is similar to that in the epidermis.

Compositions comprising a compound disclosed herein or salt thereof (or combinations thereof) may, in one or more embodiments, be administered buccally, by inhalation (e.g., spray, nebulizer, or powder puff), epidural, by injection (including intraarticular, intravenous, intracoronary, subcutaneous, intramyocardial, intraperitoneal, intramuscular, intravascular or infusion), intradermal, intraperitoneal, intrapulmonary, intraarticular (e.g., injection), nasally, orally, parenterally, rectally, sublingually, topically, transdermally, vaginally, or via an implanted reservoir.

In some embodiments, pharmaceutical compositions of the disclosure may be suitable for topical or transdermal administration.

Non-limiting examples of dosage forms for topical or transdermal administration of a compound disclosed herein or salt thereof include creams, drops, lotions, emulsions, foams, gels, inhalants, mousses, ointments, pastes, patches, powders, solutions, or sprays.

In some embodiments the compound is micronized when provided as a powder or as a suspension. In some embodiments, the compound comprises nanoparticles.

In some embodiments, compositions comprising a novel compound disclosed herein or salt thereof (or combinations thereof) may be administered to young children. In some embodiments, compositions comprising a compound of the disclosure or salt thereof (or combinations thereof) may be administered to adolescents or teenagers. In some embodiments, compositions comprising a compound of the disclosure or salt thereof (or combinations thereof) may be administered to adults.

For drug candidates for oral delivery, a higher bioavailability can translate into a lower dosage and potentially fewer side effects, e.g., in the alimentary canal. For drug candidates for oral delivery, in some embodiments, a plasma concentration higher than the free EC50 for BD 2 for a sufficient period to have a therapeutic effect, e.g., in some embodiments a period of several hours can translate into an effective drug. In some embodiments, oral delivery provides a plasma concentration over the free EC50 for BD 2 for a period of about 4 or more hours. In some embodiments, it is for a period of about 6 or more hours, or for a period of about 8 or more hours, or for a period of about 12 or more hours, or for a period of about 15 or more hours. In some embodiments the plasma concentration over the free EC50 for BD 2 is between about 4 to about 15 hours or about 6 to about 12 hours. In one or more embodiments, a therapeutically effective amount of drug is applied once a day. In some embodiments, it is applied two times a day, e.g., where the period in which the plasma concentration is higher than the free EC50 is less than 12 hours or less than 9 hours or less than 6 hours. In some embodiments, it is applied 3 times a day.

In some embodiments, compounds of the disclosure exhibit a microsomal half-life of >about 20, >about 30, >about 40, >about 50, >about 60, >about 80, >about 100, or >about 120 minutes. In some embodiments, compounds of the disclosure exhibit a plasma half-life following IV dosing of >about 20, >about 30, >about 40, >about 50, >about 60, >about 80, >about 100, or >about 120 minutes. In some embodiments microsomal half-life is between about 40 to about 70 minutes or between about 15 to about 70 minutes.

In some embodiments, compounds of the disclosure exhibit a thermodynamic solubility in FaSSIF pH 6.5 buffer of >about 10, >about 50, >about 100, >about 150, >about 200, >about 250, >about 300, >about 400, >about 500, or >about 600 PM. In some embodiments the thermodynamic solubility is between about 200 to about 1250 PM, or between about 5 to about 1250 μM.

In some embodiments, compounds of the disclosure exhibit a bioavailability of >about 10%, >about 12%, >about 20%, >about 25%, >about 30%, >about 40%, >about 50%, >about 55%>about 60%, >about 65%, or >about 70%. As used herein, bioavailability is the fraction of administered drug that reaches the systemic circulation (blood). In some embodiments, BET BDII selective protein inhibitors exhibit an IL-22 IC50 of <about 250 nM, <about 100 nM, <about 50 nM, <about 25 nM, or <about 20 nM and/or an IL-17A IC50 of <about 250 nM, <about 100 nM, <about 75 nM, <about 50 nM, or <about 20 nM. In some embodiments, BET BDII selective protein inhibitors exhibit an IL-22 IC50 of <about 50 nM and or an IL-17A IC50 of <about 10 nM. In some embodiments, BET BDII selective protein inhibitors exhibit an IL-22 IC50 of <about 20 nM and or an IL-17A IC50 of <about 7 nM. In some embodiments, BET BDII selective protein inhibitors exhibit an IL-22 IC50 of <about 20 nM and or an IL-17A IC50 of <about 40 nM. In some embodiments, BET BDII selective protein inhibitors exhibit an IL-22 IC50 of <about 15 nM and or an IL-17A IC50 of <about 40 nM. In some embodiments, BET BDII selective protein inhibitors exhibit an IL-22 IC50 of between about 100 nM and about 10 nM. In some embodiments, BET BDII selective protein inhibitors exhibit an IL-17A IC50 of between about 125 nM and about 10 nM. In some embodiments, the IC50 is a mean of two or more measurements. In some embodiments there is provided a range between any two numbers of the same type of measurement.

Non-limiting embodiments disclosed herein include:
1. A compound of formula (I), or a pharmaceutically acceptable salt or N-oxide thereof: [symbol]
wherein:
Ring A is independently selected from phenyl, 5-membered heterocyclyl, 6-membered heterocyclyl, 9-membered bicyclic heterocyclyl, and 10-membered bicyclic heterocyclyl;
X is independently selected from O and NR⁹;
Z is independently selected from N and CR¹⁰;
R^{1a} and R^{1b} are each independently selected from H, C₁-C₄-alkyl, C₂-C₄-alkenyl, C₂-C₄-alkynyl, C₀-C₄-haloalkyl, C₀-C₄-alkylene-R^{1c}, wherein R^{1c} is independently selected from C₃-C₆ cycloalkyl, 4-membered heterocyclyl, 5-membered heterocyclyl, and 6-membered heterocyclyl; wherein R^{1c} is optionally substituted with from 1 to 4 R^{1d}; or R^{1a} and R^{1b} together with the nitrogen atom to which they are attached form a 5- to 8-membered heterocycloalkyl group optionally substituted with from 1 to 4 R^{1e};
R² is independently selected from —CONR^{2a}R^{2b}, —NR^{2a}COR^{2g}, 5-membered heterocyclyl, 6-membered heterocyclyl, and phenyl, wherein the 5-membered heterocyclyl, and 6-membered heterocyclyl groups may be optionally substituted with from 1 to 4 R^{2c} and wherein the phenyl group may be optionally substituted with from 1 to 5 R^{2c};
   wherein R^{2a} and R^{2b} are each independently selected from H, C₁-C₄-alkyl, C₂-C₄-alkenyl, C₂-C₄-alkynyl, C₁-C₄-haloalkyl, C₀-C₄-alkylene-R^{2d}; wherein R^{2d} is independently selected from C₃-C₆ cycloalkyl, 4-membered heterocyclyl, 5-membered heterocyclyl, and 6-membered heterocyclyl, wherein R^{2d} is optionally substituted with from 1 to 4 R^{2e};
   or R^{2a} and R^{2b} together with the nitrogen atom to which they are attached form a 5- to 8-membered heterocycloalkyl group optionally substituted with from 1 to 4 R²;
   wherein R^{2g} is independently selected from C₁-C₄-alkyl, C₂-C₄-alkenyl, C₂-C₄-alkynyl, C₁-C₄-haloalkyl, C₀-C₄-alkylene-R^{2d}; wherein R^{2d} is independently selected from C₃-C₆ cycloalkyl, 4-membered heterocyclyl, 5-membered heterocyclyl, and 6-membered heterocyclyl, wherein R^{2d} is optionally substituted with from 1 to 4 R^{2e};
   or R^{2a} and R^{2g} together with the atoms to which they are attached form a 5- to 8-membered heterocycloalkyl group optionally substituted with from 1 to 4 R^{2f};
R^{1d}, R^{1e}, R^{2c}, R^{2e} and R^{2f} are each independently at each occurrence selected from ═O, ═S, halo, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₄-cycloalkyl, 4-membered heterocyclyl, 5-membered heterocyclyl, and 6-membered heterocycloalkyl;
R³ is independently selected from H, C₁-C₄-alkyl, C₂-C₄-alkenyl, C₂-C₄-alkynyl, C₁-C₄-haloalkyl, C₂-C₄-haloalkenyl, C₁-C₄-alkylene-OR⁷, C₀-C₄-alkylene-S(O)₂R⁶, C₀-C₄-alkylene-CONR⁶R⁶, C₃-C₄-cycloalkyl, 4-membered heterocyclyl, 5-membered heterocyclyl, and 6-membered heterocyclyl;
R⁴ is independently at each occurrence selected from ═O, ═S, halo, nitro, cyano, C₀-C₄-alkylene-NR⁵R⁶, C₀-C₄-alkylene-OR⁷, SR⁶, SOR⁶, C₀-C₄-alkylene-S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl, C₁-C₄-alkyl-S(O)₂R⁶, C₂-C₄-alkenyl, C₂-C₄-alkynyl, C₁-C₄-haloalkyl, C₃-C₄-cycloalkyl and 4-membered heterocycloalkyl;
R⁵ is independently at each occurrence selected from H, C₁-C₄-alkyl, C(O)—C₁-C₄-alkyl and S(O)₂—C₁-C₄-alkyl; and
R⁶ is independently at each occurrence selected from H and C₁-C₄-alkyl; or where two R⁶ groups are attached to the same nitrogen, those two R⁶ groups together with the nitrogen atom to which they are attached optionally form a 5- to 8-membered-heterocycloalkyl group optionally substituted with from 1 to 4 R¹;
or R⁵ and R⁶ together with the nitrogen atom to which they are attached form a C₅-C₈-heterocycloalkyl group optionally substituted with from 1 to 4 R¹;
R⁷ is independently at each occurrence selected from H, C₁-C₄-alkyl, C(O)—C₁-C₄-alkyl and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, CO₂R, C(O)R⁶, CONR⁶R⁶, C₁-C₄-alkyl, C₂-C₄-alkenyl, C₂-C₄-alkynyl, C₁-C₄-haloalkyl, C₃-C₄-cycloalkyl and 4-membered heterocycloalkyl;
R⁹ is independently selected from H, C₁-C₄-alkyl, C₂-C₄-alkenyl, C₂-C₄-alkynyl, C₁-C₄-haloalkyl, C₂-C₄-haloalkenyl and C₃-C₄-cycloalkyl;
R¹⁰ is independently selected from H, halo, C₁-C₄-alkyl, C₂-C₄-alkenyl, C₂-C₄-alkynyl, C₁-C₄-haloalkyl, C₂-C₄-haloalkenyl, C₀-C₄-alkylene-OR⁷ and C₃-C₆-cycloalkyl; and
m is an integer selected from 0, 1, 2, 3 and 4;
wherein any of the aforementioned alkyl, alkylene, alkenyl, or cyclopropyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a}, and S(O)₂R^{a}; wherein R^{a} is independently at each occurrence selected from H and C₁-C₄-alkyl; and R^{b} is independently at each occurrence selected from H, C₁-C₄-alkyl, C(O)—C₁-C₄-alkyl and S(O)₂-C₁-C₄-alkyl.
2. The compound of embodiment 1, or a pharmaceutically acceptable salt or N-oxide thereof, having a structure according to Formula (IIA): [symbol]
3. The compound of embodiment 1 or embodiment 2, or a pharmaceutically acceptable salt or N-oxide thereof, wherein Ring A is 5-membered heteroaryl.
4. A compound of embodiment 1 or embodiment 2, or a pharmaceutically acceptable salt or N-oxide thereof, wherein when Ring A is phenyl.
5. A compound of any preceding embodiment, or a pharmaceutically acceptable salt or N-oxide thereof, wherein Z is CR¹⁰.
6. A compound of any preceding embodiment, or a pharmaceutically acceptable salt or N-oxide thereof, wherein Z is N.
7. A compound of any preceding embodiment, or a pharmaceutically acceptable salt or N-oxide thereof, wherein X is O.
8. A compound of any preceding embodiment, or a pharmaceutically acceptable salt or N-oxide thereof, wherein R^{1a} is C₁-C₄-alkyl and R^{1b} is H.
9. A compound of any preceding embodiment, or a pharmaceutically acceptable salt or N-oxide thereof, wherein R² is —CONR^{2a}R^{2b}.
10. A compound of embodiment 9, or a pharmaceutically acceptable salt or N-oxide thereof, wherein R^{2a} is C₁-C₄-alkyl and R^{2b} is H.
11. A compound of any preceding embodiment, or a pharmaceutically acceptable salt or N-oxide thereof, wherein R³ is C₁-C₄-alkyl.
12. A compound of any preceding embodiment, or a pharmaceutically acceptable salt or N-oxide thereof, wherein R⁴ is independently selected at each occurrence from C₁-C₄-alkyl, halo, cyano, C₁-C₄-haloalkyl, and C₀-C₄-alkylene-OR⁷.
13. A compound of any preceding embodiment, or a pharmaceutically acceptable salt or N-oxide thereof, wherein m is an integer selected from 0 or 1.
14. A compound of embodiment 1, or a pharmaceutically acceptable salt or N-oxide thereof, wherein the compound according to formula (I) is selected from:

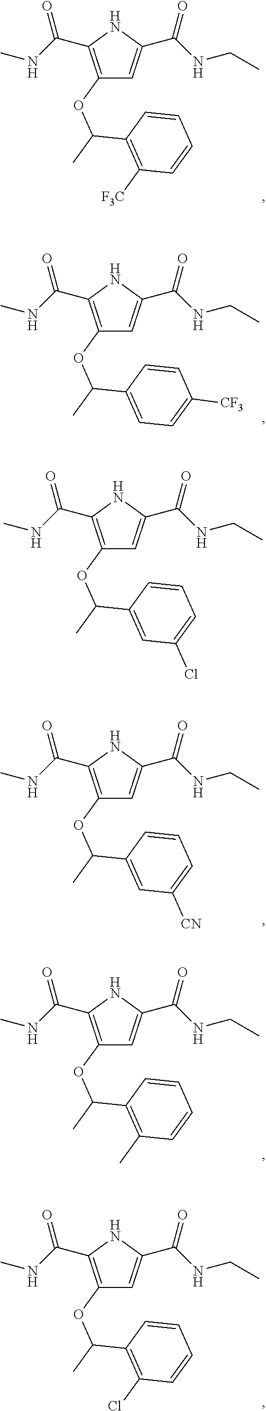

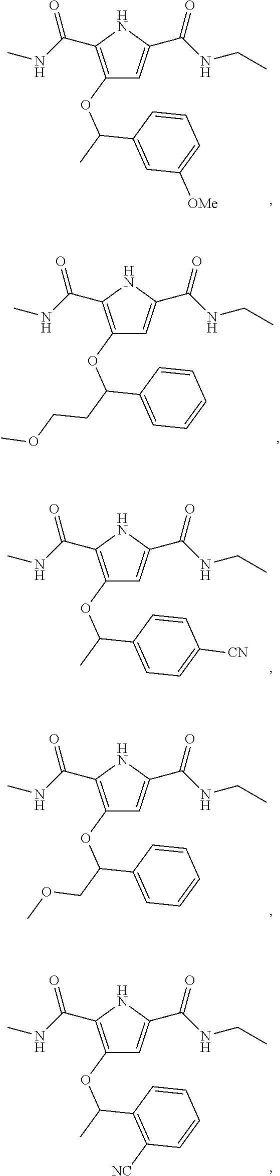

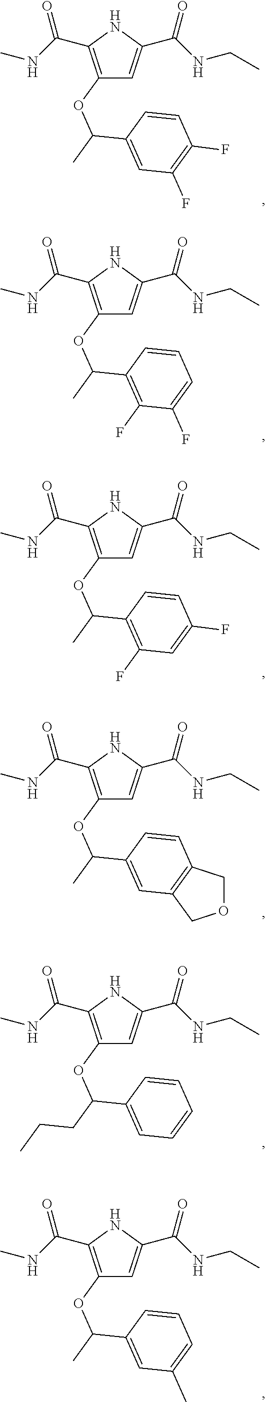

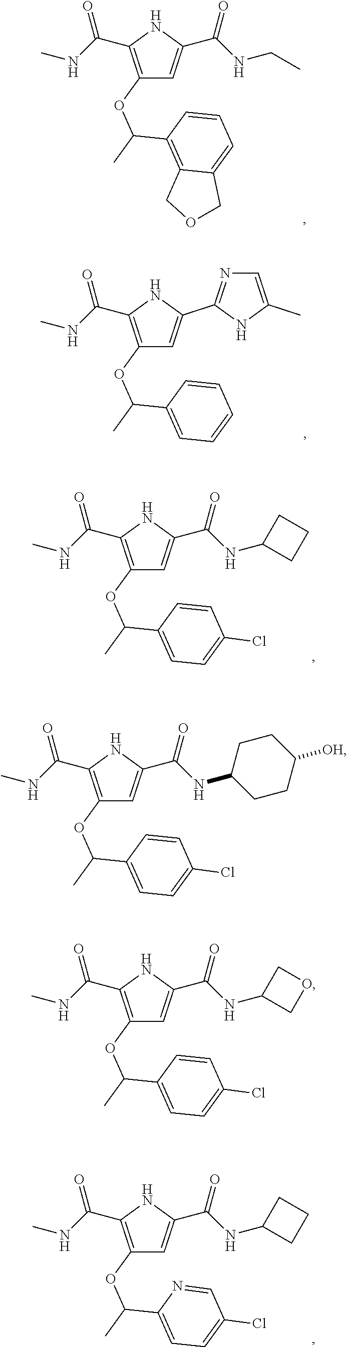

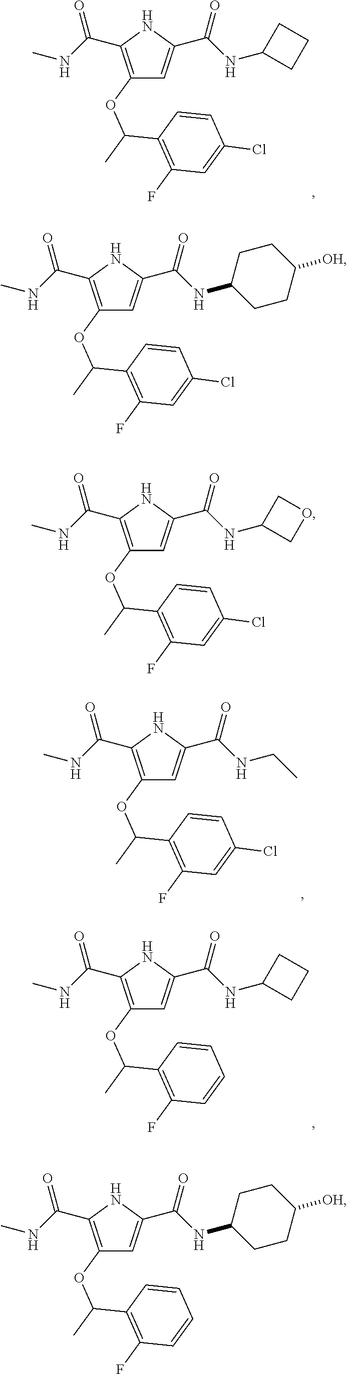

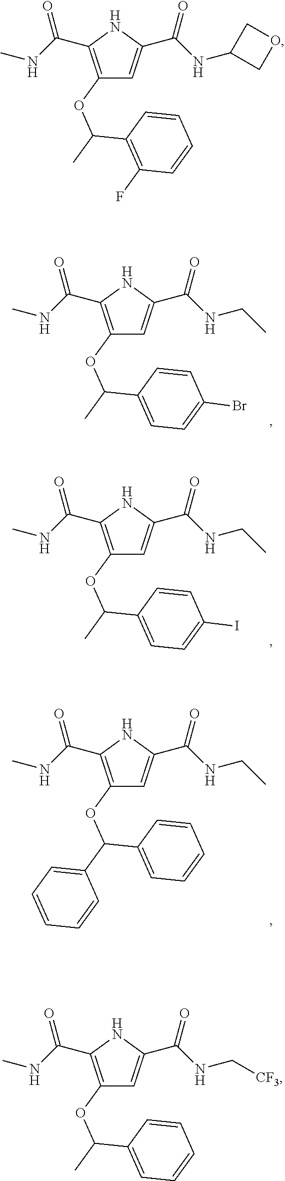

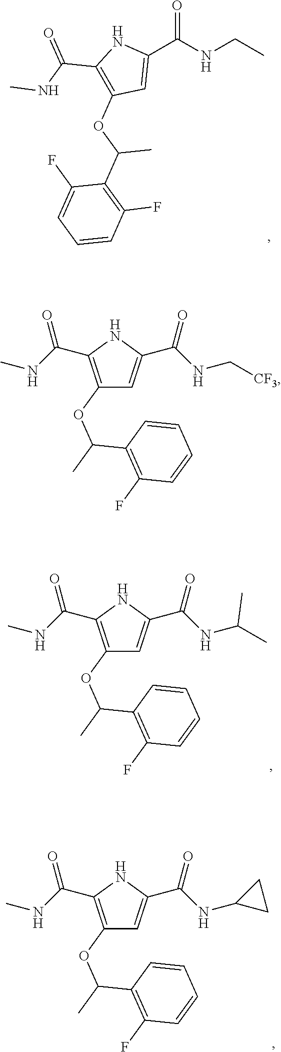

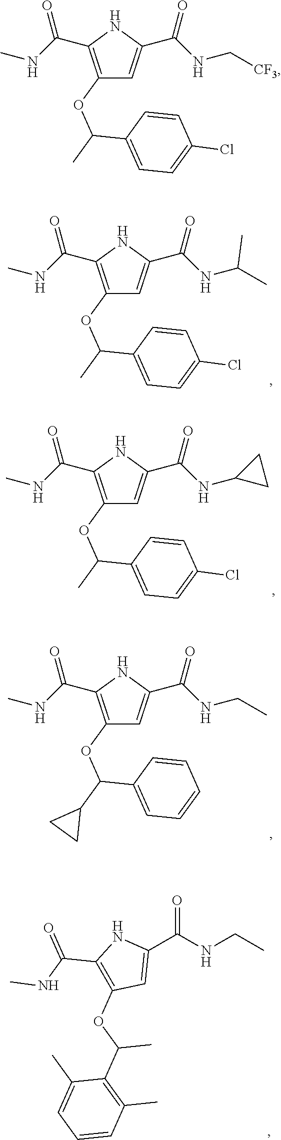

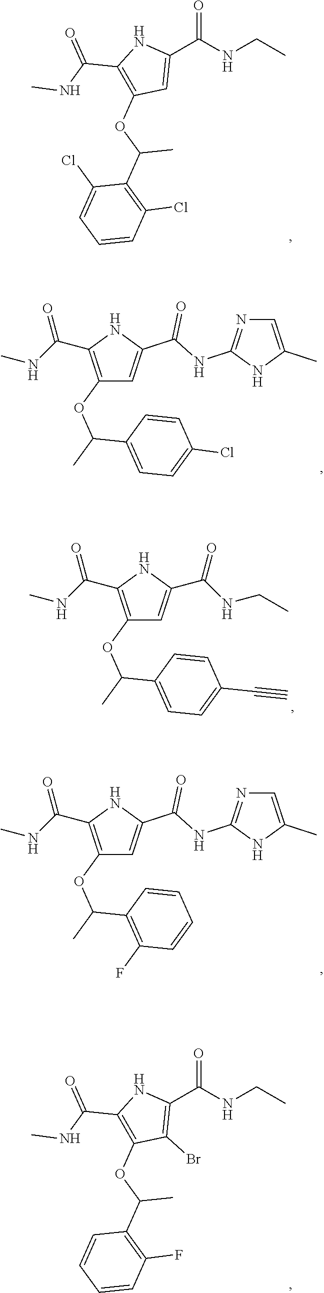

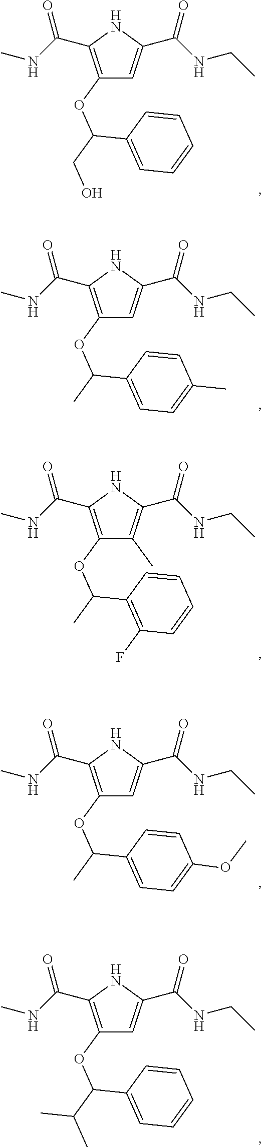

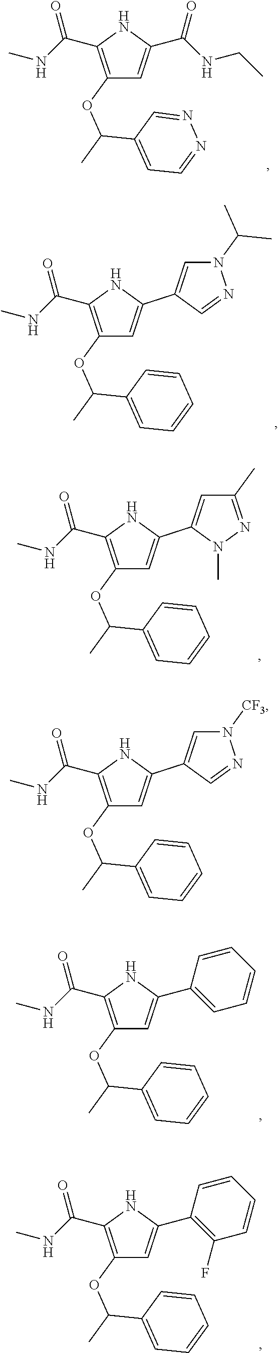

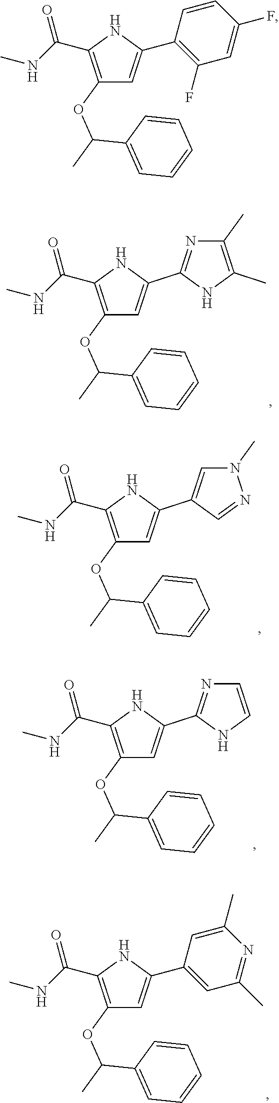

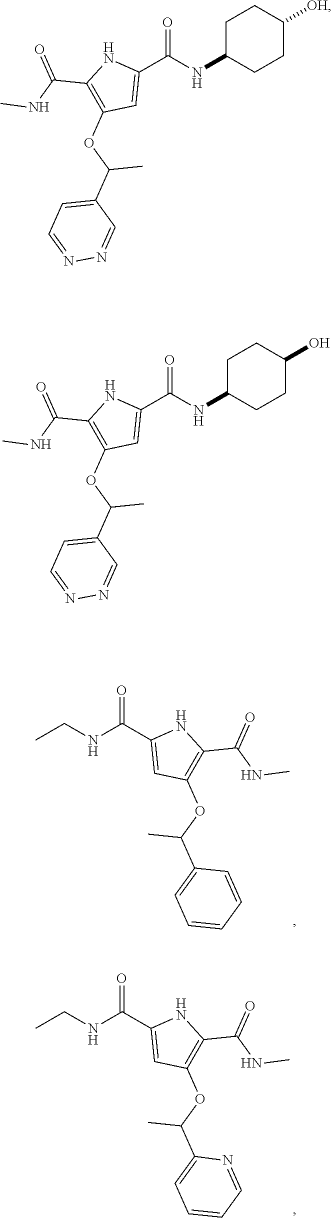

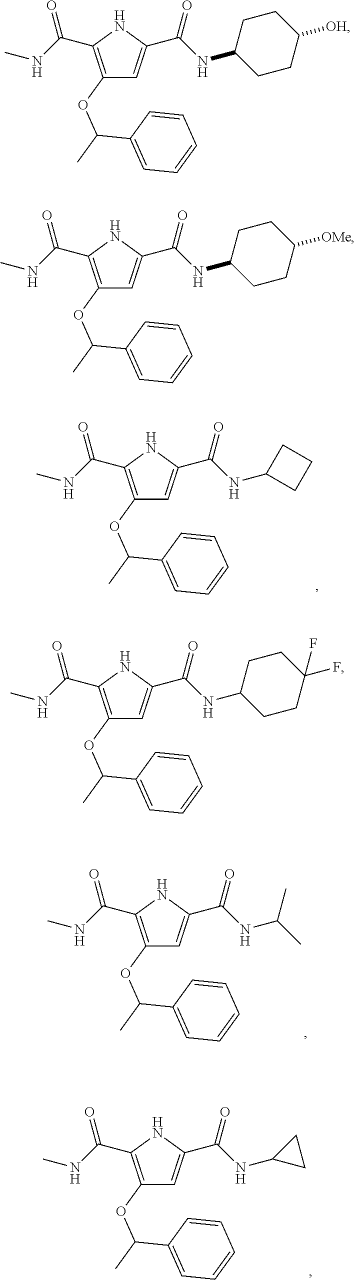

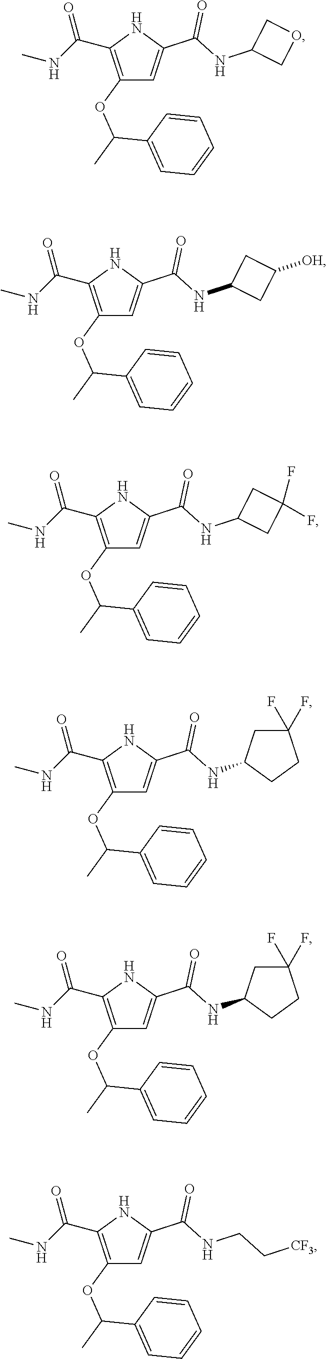

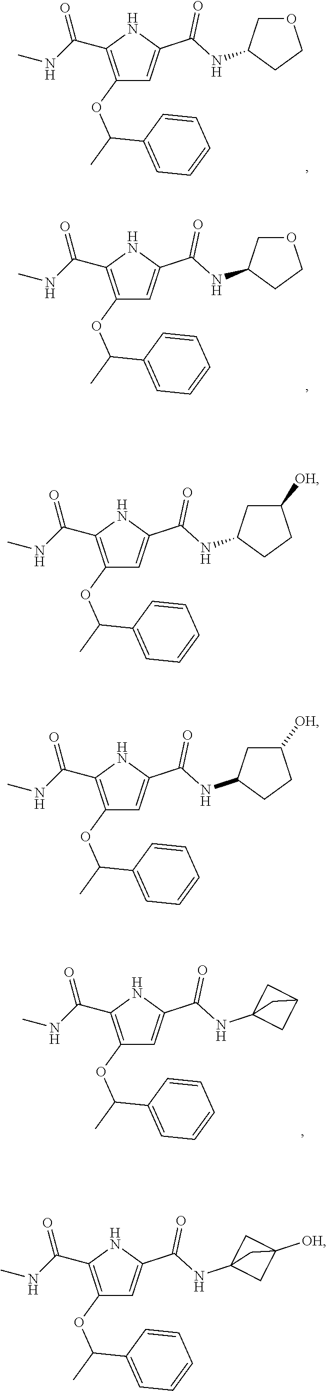

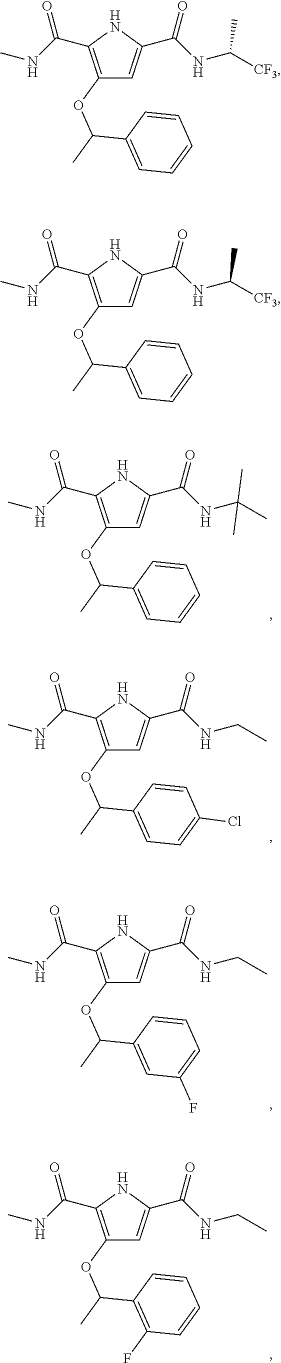

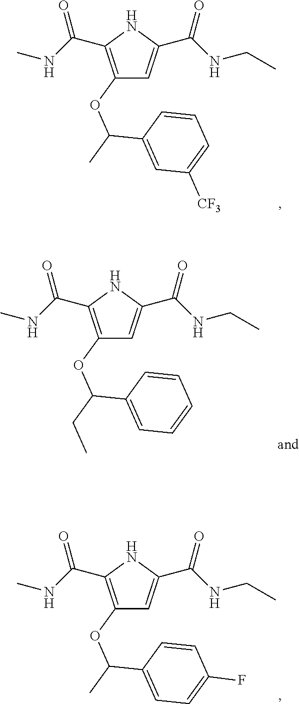

or a stereoisomer or a mixture of stereoisomers thereof.
15. A compound of embodiment 2, or a pharmaceutically acceptable salt or N-oxide thereof, wherein the compound according to formula (IIA) is selected from:

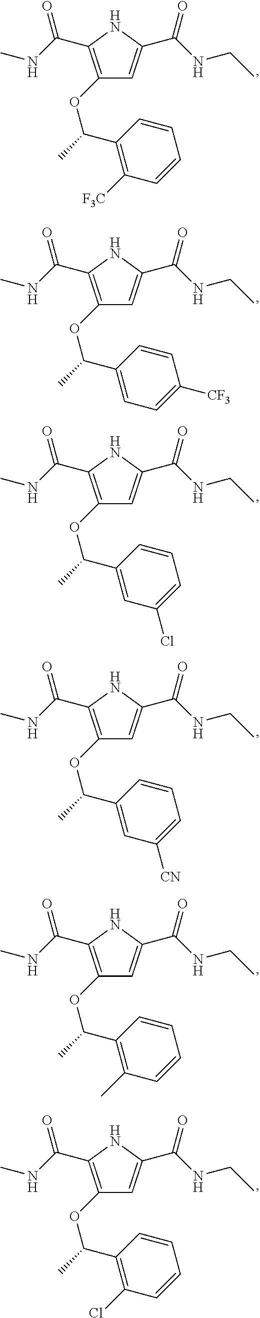

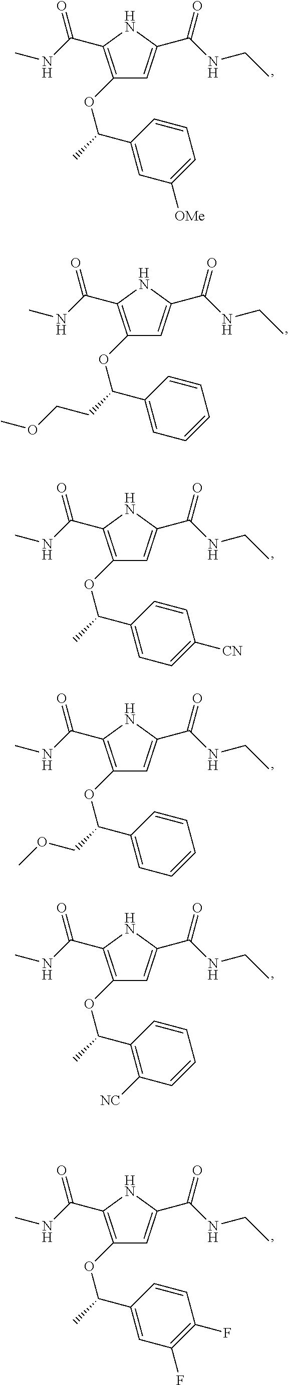

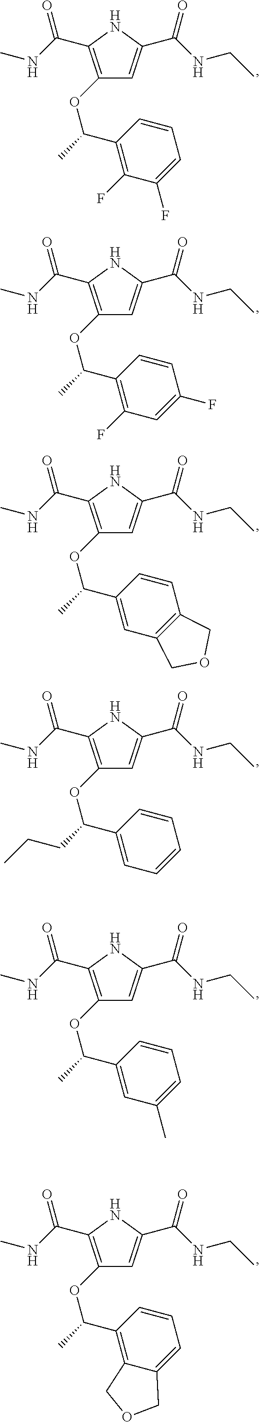

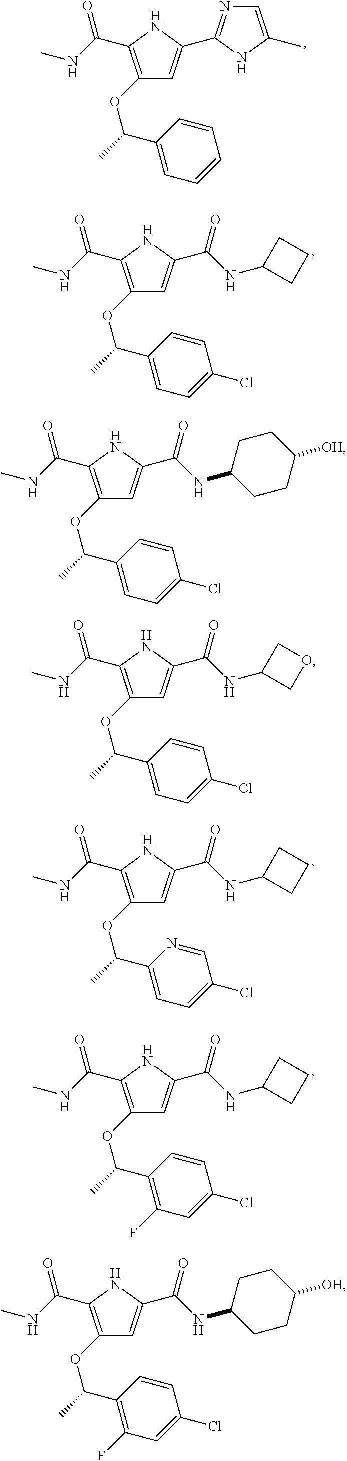

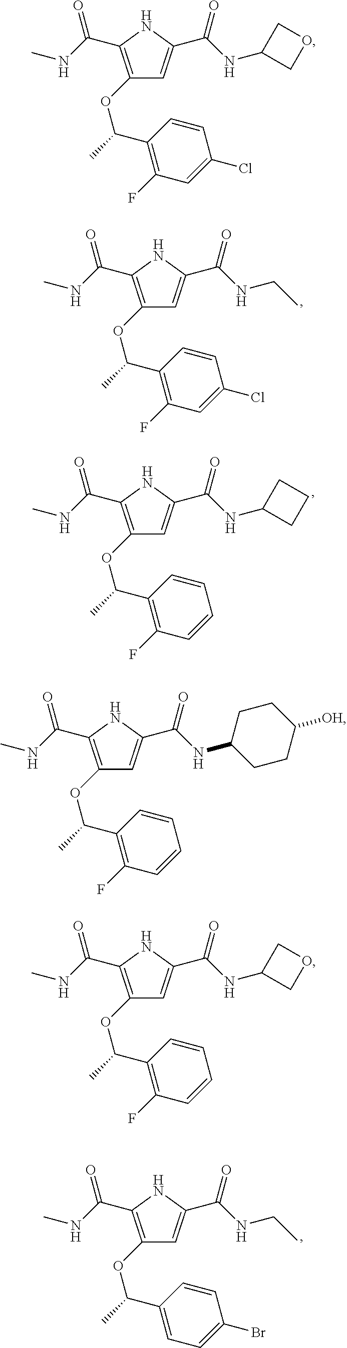

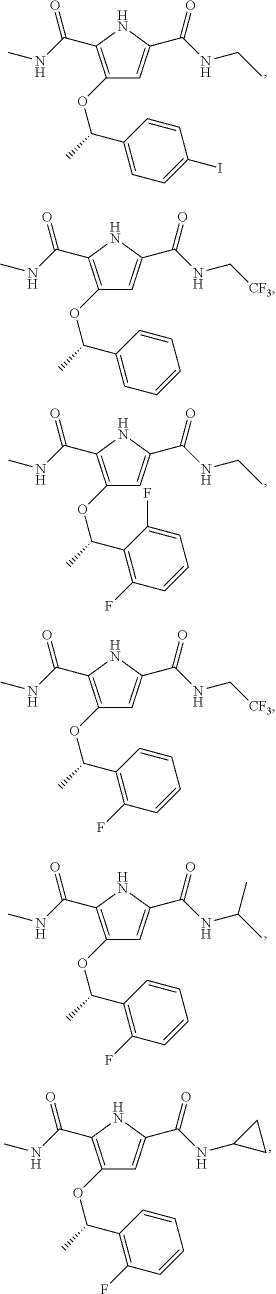

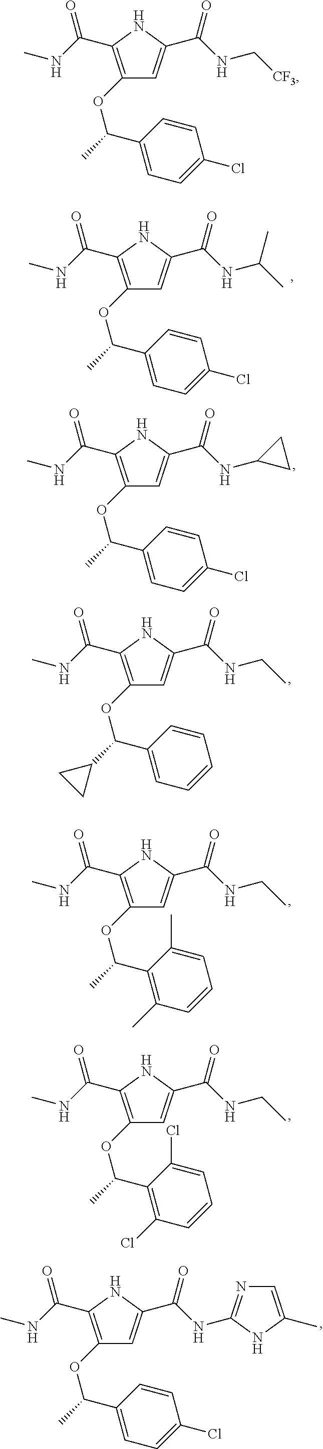

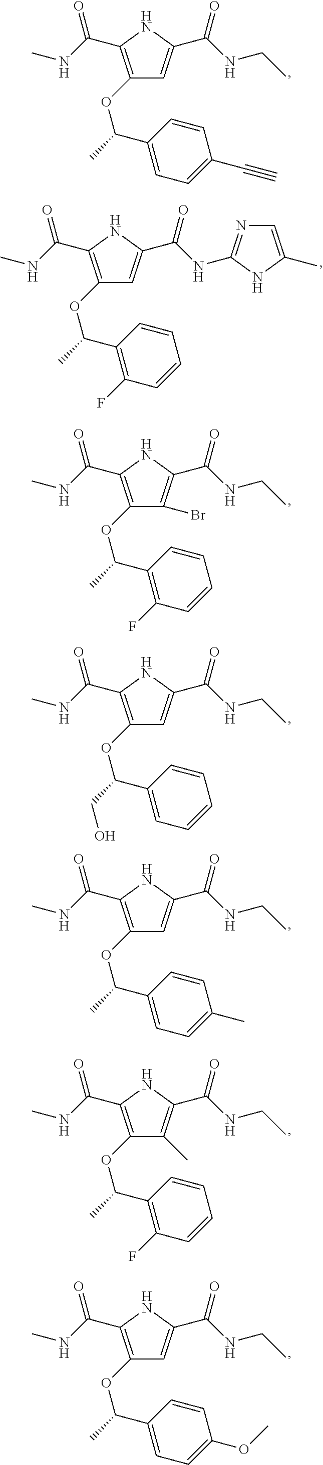

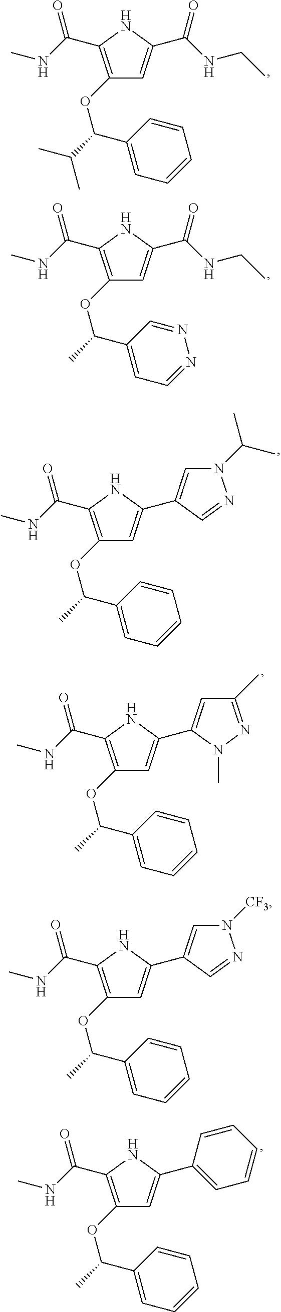

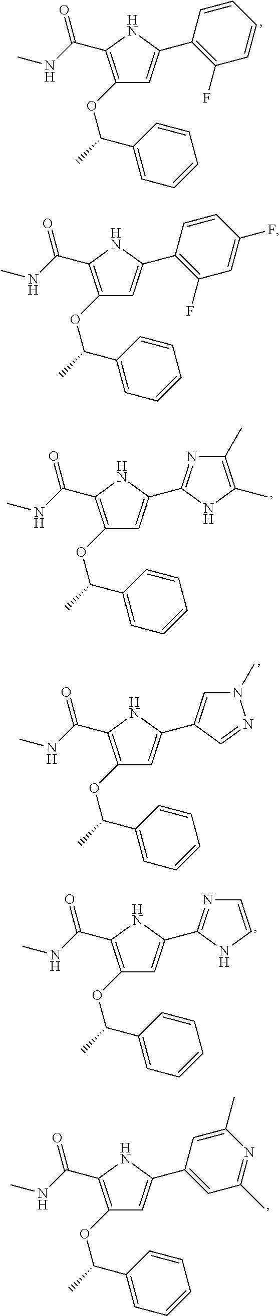

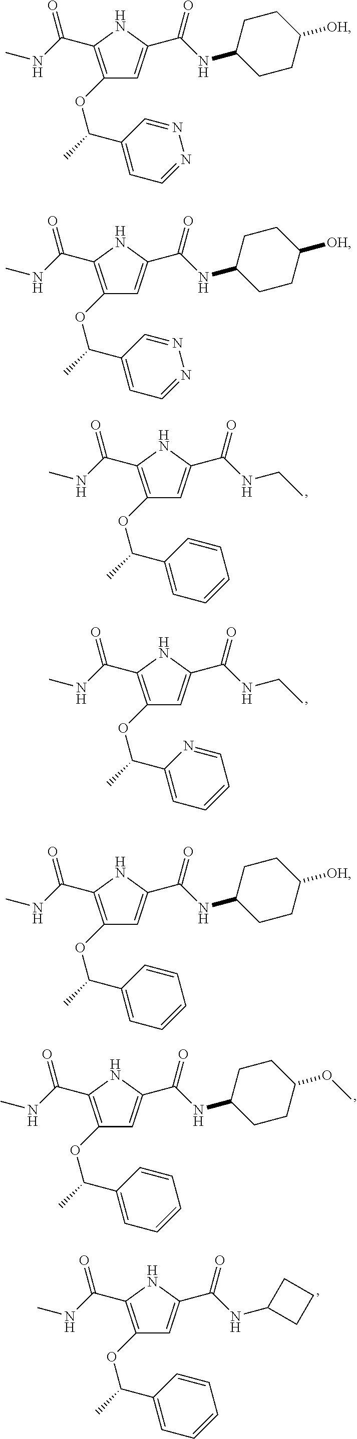

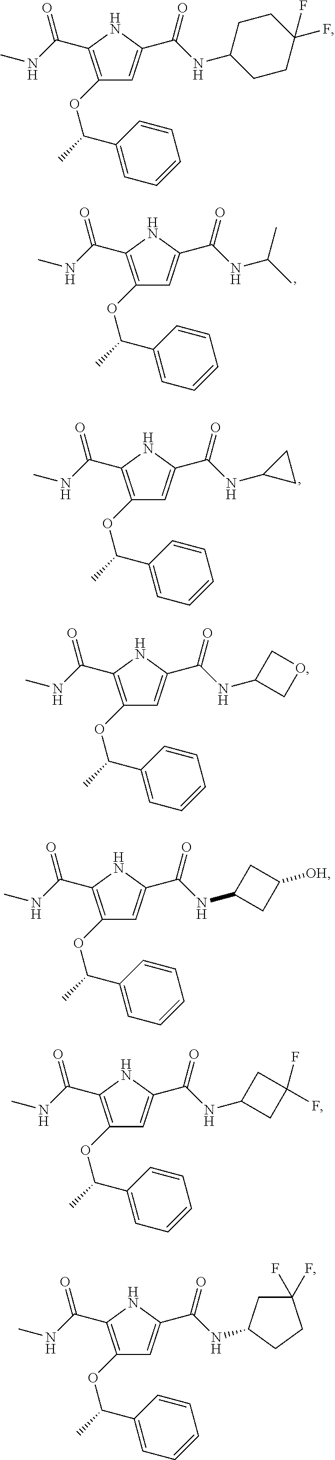

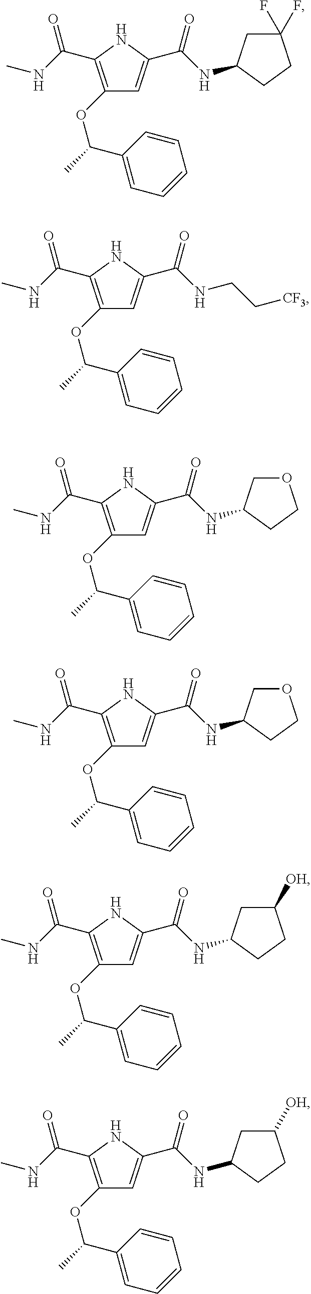

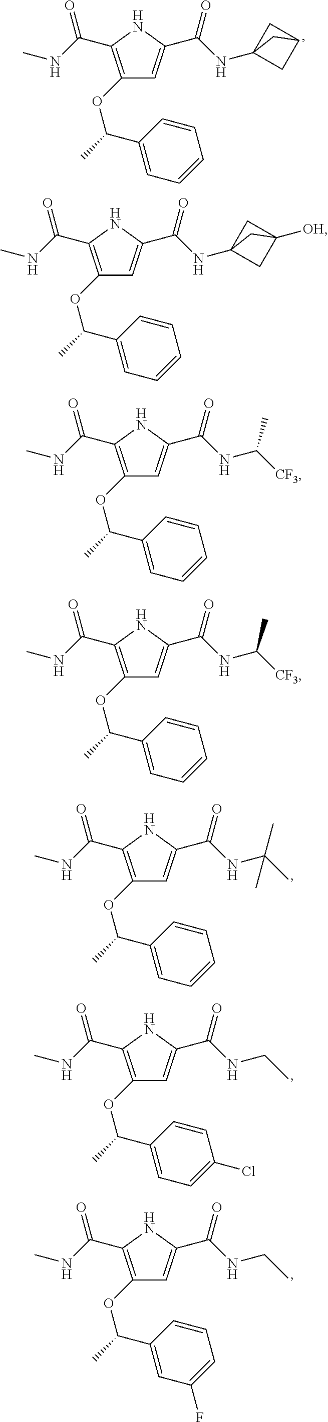

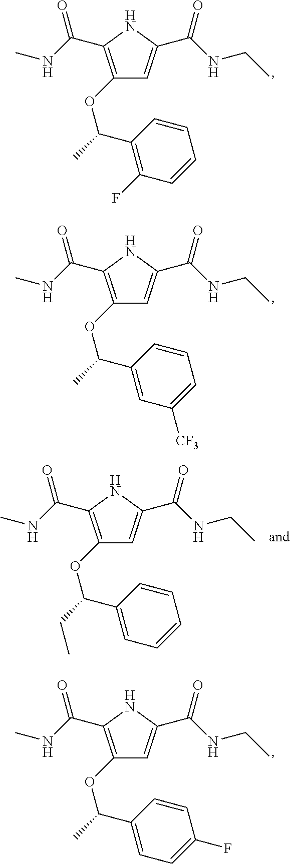

or a stereoisomer thereof.
16. A pharmaceutical composition comprising a compound of any one of embodiments 1 to 15, or a pharmaceutically acceptable salt or N-oxide thereof, and one or more pharmaceutically acceptable excipients.
17. A compound of any one of embodiments 1 to 15, or a pharmaceutically acceptable salt or N-oxide thereof, for use as a medicament.
18. A compound of any one of embodiments 1 to 15, or a pharmaceutically acceptable salt or N-oxide thereof, for use in treating a disease or disorder selected from an inflammatory disorder, an immune disorder, and an autoimmune disorder.
19. A compound of any one of embodiments 1 to 15, or a pharmaceutically acceptable salt or N-oxide thereof, for use in treating a cancer.
20. A compound of any one of embodiments 1 to 15, a tautomer, a stereoisomer or a mixture of stereoisomers, a pharmaceutically acceptable salt, a hydrate, a deuterated derivative, or N-oxide thereof, for use in treating a disease or disorder, wherein the disease or disorder is a joint or joint-related disease or disorder.
21. The compound for use according to embodiment 20, wherein the joint or joint-related disease or disorder is selected from arthritis, bursitis, Ehlers-Danlos syndrome, epicondylitis, Felty Syndrome, gouty arthritis, psoriatic arthritis, osteoarthritis, rheumatoid arthritis, Still's disease, tenosynovitis, synovitis, Sjögren's Syndrome, Lyme disease, Whipple disease, bone cancer, lupus, and other autoimmune joint disorders.
22. The compound for use according to embodiment 20 or 21, wherein the joint or joint-related disease or disorder comprises an arthritis.
23. The compound for use according to embodiment 22, wherein the arthritis comprises rheumatoid arthritis.
24. The compound for use according to any one of embodiments 20-23, wherein the disorder is an arthritis and upon administration of a therapeutically effective amount of the compound, tautomer, stereoisomer or mixture of stereoisomers, pharmaceutically acceptable salt, hydrate, deuterated derivative, or N-oxide thereof there is a therapeutic effect associated with reduction in inflammation.
25. The compound for use according to embodiment 24, wherein the therapeutic effect associated with a reduction in inflammation is a reduction in thickness or girth of a joint or limb.
26. The compound for use according to embodiment 24 or 25, wherein there is reduction in arthritic scoring or severity, and
   wherein the reduction in arthritic scoring or severity is a reduction in:
   (a) definite redness and swelling of an ankle/wrist or apparent redness and swelling limited to individual digits, regardless of the number of affected digits;
   (b) severe redness and swelling of an ankle/wrist;
   (c) redness and swelling of the entire appendage including digits; and/or
   (d) maximally inflamed limb with involvement of multiple joints.
27. The compound for use according to any one of embodiments 24 to 26, wherein the reduction is dose dependent.
28. The compound for use according to any one of embodiments 24 to 27, wherein the reduction is by >about 50%, or the reduction is about 50%, or the reduction is between about 20% to about 70%.
29. The compound for use according to any one of embodiments 18 and 20 to 28, wherein the compound, tautomer, stereoisomer or mixture of stereoisomers, pharmaceutically acceptable salt, hydrate, deuterated derivative, or N-oxide thereof is in the form of a pharmaceutical composition which further comprises a pharmaceutically acceptable carrier.
30. The compound for use according to embodiment 29, wherein the compound, tautomer, stereoisomer, pharmaceutically acceptable salt, hydrate, and/or deuterated derivative thereof is formulated as a suspension or partial suspension in the composition.
31. The compound for use according to embodiment 30, wherein the compound, tautomer, stereoisomer or mixture of stereoisomers, pharmaceutically acceptable salt, hydrate, deuterated derivative, or N-oxide thereof is micronized.
32. The compound for use according to embodiment 30 or 31, wherein the compound, tautomer, stereoisomer or mixture of stereoisomers, pharmaceutically acceptable salt, hydrate, deuterated derivative, or N-oxide thereof is in the form of nanoparticles.
33. The compound for use according to any one of embodiments 18 and 20 to 32, wherein the compound, tautomer, stereoisomer or mixture of stereoisomers, pharmaceutically acceptable salt, hydrate, deuterated derivative, or N-oxide thereof is solubilized or partially solubilized in the composition.
34. The compound for use according to of any one of embodiments 18 and 20 to 33, wherein the compound, tautomer, stereoisomer or mixture of stereoisomers, pharmaceutically acceptable salt, hydrate, deuterated derivative, or N-oxide thereof or pharmaceutical composition is administered locally, topically or systemically.
35. The compound for use according to of any one of embodiments 18 and 20 to 34, wherein the compound, tautomer, stereoisomer or mixture of stereoisomers, pharmaceutically acceptable salt, hydrate, deuterated derivative, or N-oxide thereof or pharmaceutical composition is administered orally.
36. The compound for use according to of any one of embodiments 18 and 20 to 35, wherein the compound, tautomer, stereoisomer or mixture of stereoisomers, pharmaceutically acceptable salt, hydrate, deuterated derivative, or N-oxide thereof or pharmaceutical composition has activity against one or more BET domains.
37. A compound of any one of embodiments 1 to 15, a tautomer, a stereoisomer or a mixture of stereoisomers, a pharmaceutically acceptable salt, a hydrate, a deuterated derivative, or N-oxide thereof, for use in the treatment of a joint or joint-related disease in which a therapeutic effect associated with a reduction in inflammation is achieved.
38. A compound of any one of embodiments 1 to 15, a tautomer, a stereoisomer or a mixture of stereoisomers, a pharmaceutically acceptable salt, a hydrate, a deuterated derivative, or N-oxide thereof, for use in the treatment of an arthritic disease in which a therapeutic effect associated with a reduction in inflammation is achieved.
39. A compound of any one of embodiments 1 to 15, a tautomer, a stereoisomer or a mixture of stereoisomers, a pharmaceutically acceptable salt, a hydrate, a deuterated derivative, or N-oxide thereof, for use in the treatment of a fibrotic disease or disorder.
40. The compound for use according to embodiment 39, wherein the disease or disorder is renal fibrosis.
41. The compound for use according to embodiment 39 or 40, wherein, upon administration of a therapeutically effective amount of the compound, tautomer, stereoisomer or mixture of stereoisomers, pharmaceutically acceptable salt, hydrate, deuterated derivative, or N-oxide thereof there is a therapeutic effect associated with a reduction in fibrosis.
42. The compound for use according to embodiment 41, wherein the reduction in fibrosis comprises a reduction in pathology in the kidneys.
43. The compound for use according to embodiment 42, wherein the reduction in pathology in the kidney comprises a reduction in interstitial nephritis, collagen fiber deposition, and nephropathy.
44. The compound for use according to any one of embodiments 41 to 43, wherein the reduction in fibrosis comprises a reduction in inflammatory tissue biomarkers.
45. The compound for use according to embodiment 44, wherein the inflammatory tissue biomarkers include Col1A1, TGF-b1, MCP-1, IL-1b, IL-6, and Timp1.
46. A compound of any one of embodiments 1 to 15, a tautomer, a stereoisomer or a mixture of stereoisomers, a pharmaceutically acceptable salt, a hydrate, a deuterated derivative, or N-oxide thereof, for use in the treatment of a fibrotic disease in which a therapeutic effect associated with a reduction in fibrosis is achieved.
47. A compound of any one of embodiments 1 to 15, a tautomer, a stereoisomer or a mixture of stereoisomers, a pharmaceutically acceptable salt, a hydrate, a deuterated derivative, or N-oxide thereof, for use in the treatment of renal fibrosis in which a therapeutic effect associated with a reduction in fibrosis is achieved.
48. The compound for use of any of embodiments 40 to 47, wherein the progression of fibrosis severity is slowed or retarded.
49. The compound for use of any of embodiments 40 to 47, wherein the appearance or increase of one or more indicators of fibrosis severity is slowed or retarded.

Non-limiting embodiments disclosed herein additionally include:
1. A compound of formula (I), or a pharmaceutically acceptable salt or N-oxide thereof:

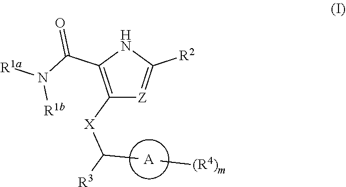

wherein:
Ring A is independently selected from phenyl, 5-membered heterocyclyl, 6-membered heterocyclyl, 9-membered bicyclic heterocyclyl, and 10-membered bicyclic heterocyclyl;
X is independently selected from O and NR⁹;
Z is independently selected from N and CR¹⁰;
R^{1a} and R^{1b} are each independently selected from H, C₁-C₄-alkyl, C₂-C₄-alkenyl, C₂-C₄-alkynyl, C₁-C₄-haloalkyl, C₀-C₄-alkylene-R^{1c}, wherein R^{1c} is independently selected from C₃-C₆ cycloalkyl, 4-membered heterocyclyl, 5-membered heterocyclyl, and 6-membered heterocyclyl; wherein R^{1c} is optionally substituted with from 1 to 4 R^{1d};
or R^{1a} and R^{1b} together with the nitrogen atom to which they are attached form a 5- to 8-membered heterocycloalkyl group optionally substituted with from 1 to 4 R^{1e};
R² is independently selected from —CONR^{2a}R^{2b}, —NR^{2a}COR^{2g}, 5-membered heterocyclyl, 6-membered heterocyclyl, and phenyl, wherein the 5-membered heterocyclyl, and 6-membered heterocyclyl groups may be optionally substituted with from 1 to 4 R^{2c} and wherein the phenyl group may be optionally substituted with from 1 to 5 R^{2c}:
   wherein R^{2a} and R^{2b} are each independently selected from H, C₁-C₄-alkyl, C₂-C₄-alkenyl, C₂-C₄-alkynyl, C₁-C₄-haloalkyl, C₀-C₄-alkylene-R^{2d}; wherein R^{2d} is independently selected from C₃-C₆ cycloalkyl, 4-membered heterocyclyl, 5-membered heterocyclyl, and 6-membered heterocyclyl, wherein R^{2d} is optionally substituted with from 1 to 4 R^{2e};
   or R^{2a} and R^{2b} together with the nitrogen atom to which they are attached form a 5- to 8-membered heterocycloalkyl group optionally substituted with from 1 to 4 R^{2e};
   wherein R^{2g} is independently selected from C₁-C₄-alkyl, C₂-C₄-alkenyl, C₂-C₄-alkynyl, C₁-C₄-haloalkyl, C₀-C₄-alkylene-R^{2d}; wherein R^{2d} is independently selected from C₃—C cycloalkyl, 4-membered heterocyclyl, 5-membered heterocyclyl, and 6-membered heterocyclyl, wherein R^{2d} is optionally substituted with from 1 to 4 R^{2e};
   or R^{2a} and R^{2g} together with the atoms to which they are attached form a 5- to 8-membered heterocycloalkyl group optionally substituted with from 1 to 4 R^{2f};
R^{1d}, R^{1e}, R^{2c}, R^{2e} and R^{2f} are each independently at each occurrence selected from ═O, ═S, halo, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₄-cycloalkyl, 4-membered heterocyclyl, 5-membered heterocyclyl, and 6-membered heterocycloalkyl;
R³ is independently selected from H, C₁-C₄-alkyl, C₂-C₄-alkenyl, C₂-C₄-alkynyl, C₁-C₄-haloalkyl, C₂-C₄-haloalkenyl, C₁-C₄-alkylene-OR⁷, C₀-C₄-alkylene-S(O)₂R⁶, C₀-C₄-alkylene-CONR⁶R⁶, C₃-C₄-cycloalkyl, 4-membered heterocyclyl, 5-membered heterocyclyl, and 6-membered heterocyclyl;
R⁴ is independently at each occurrence selected from ═O, ═S, halo, nitro, cyano, C₀-C₄-alkylene-NR⁵R⁶, C₀-C₄-alkylene-OR⁷, SR⁶, SOR⁶, C₀-C₄-alkylene-S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl, C₁-C₄-alkyl-S(O)₂R⁶, C₂-C₄-alkenyl, C₂-C₄-alkynyl, C₁-C₄-haloalkyl, C₃-C₄-cycloalkyl and 4-membered heterocycloalkyl;
R⁵ is independently at each occurrence selected from H, C₁-C₄-alkyl, C(O)—C₁-C₄-alkyl and S(O)₂—C₁-C₄-alkyl; and
R⁶ is independently at each occurrence selected from H and C₁-C₄-alkyl; or where two R⁶ groups are attached to the same nitrogen, those two R⁶ groups together with the nitrogen atom to which they are attached optionally form a 5- to 8-membered-heterocycloalkyl group optionally substituted with from 1 to 4 R⁸;
or R⁵ and R⁶ together with the nitrogen atom to which they are attached form a 5- to 8-membered heterocycloalkyl group optionally substituted with from 1 to 4 R⁸;
R⁷ is independently at each occurrence selected from H, C₁-C₄-alkyl, C(O)—C₁-C₄-alkyl and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₄-cycloalkyl and 4-membered heterocycloalkyl;
R⁹ is independently selected from H, C₁-C₄-alkyl, C₂-C₄-alkenyl, C₂-C₄-alkynyl, C₁-C₄-haloalkyl, C₂-C₄-haloalkenyl and C₃-C₄-cycloalkyl;
R¹⁰ is independently selected from H, halo, C₁-C₄-alkyl, C₂-C₄-alkenyl, C₂-C₄-alkynyl, C₁-C₄-haloalkyl, C₂-C₄-haloalkenyl, C₀-C₄-alkylene-OR⁷ and C₃-C₆-cycloalkyl; and
m is an integer selected from 0, 1, 2, 3 and 4;
wherein any of the aforementioned alkyl, alkylene, alkenyl, or cyclopropyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a}, and S(O)₂R^{a}; wherein R^{a} is independently at each occurrence selected from H and C₁-C₄-alkyl; and R^{b} is independently at each occurrence selected from H, C₁-C₄-alkyl, C(O)—C₁-C₄-alkyl and S(O)₂—C₁-C₄-alkyl.
2. The compound of embodiment 1, or a pharmaceutically acceptable salt or N-oxide thereof, having a structure according to Formula (IIA):

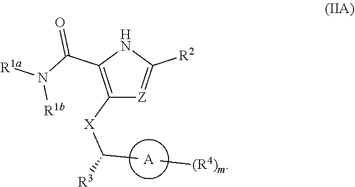

3. The compound of embodiment 1 or embodiment 2, or a pharmaceutically acceptable salt or N-oxide thereof, wherein Ring A is 5-membered heteroaryl.
4. The compound of embodiment 1 or embodiment 2, or a pharmaceutically acceptable salt or N-oxide thereof, wherein when Ring A is phenyl.
5. The compound of any one of embodiments 1 to 4, or a pharmaceutically acceptable salt or N-oxide thereof, wherein Z is CR¹⁰.
6. The compound of any one of embodiments 1 to 4, or a pharmaceutically acceptable salt or N-oxide thereof, wherein Z is N.
7. The compound of any one of embodiments 1 to 5, or a pharmaceutically acceptable salt or N-oxide thereof, wherein X is O.
8. The compound of any one of embodiments 1 to 6, or a pharmaceutically acceptable salt or N-oxide thereof, wherein R^{1a} is C₁-C₄-alkyl and R^{1b} is H.
9. The compound of any one of embodiments 1 to 7, or a pharmaceutically acceptable salt or N-oxide thereof, wherein R² is —CONR^{2a}R^{2b}.
10. The compound of embodiment 9, or a pharmaceutically acceptable salt or N-oxide thereof, wherein R^{2a} is C₁-C₄-alkyl and R^{2b} is H.
11. The compound of any one of embodiments 1 to 10, or a pharmaceutically acceptable salt or N-oxide thereof, wherein R³ is C₁-C₄-alkyl.
12. The compound of any one of embodiments 1 to 11, or a pharmaceutically acceptable salt or N-oxide thereof, wherein R⁴ is independently selected at each occurrence from C₁-C₄-alkyl, halo, cyano, C₁-C₄-haloalkyl, and C₀-C₄-alkylene-OR⁷.
13. The compound of any one of embodiments 1 to 12, or a pharmaceutically acceptable salt or N-oxide thereof, wherein m is an integer selected from 0 or 1.
14. The compound of embodiment 1, or a pharmaceutically acceptable salt or N-oxide thereof, wherein the compound according to formula (I) is selected from:

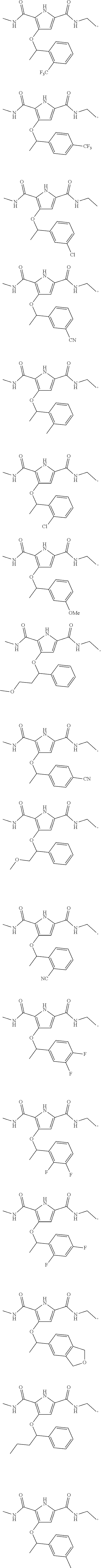

[chemical structure]

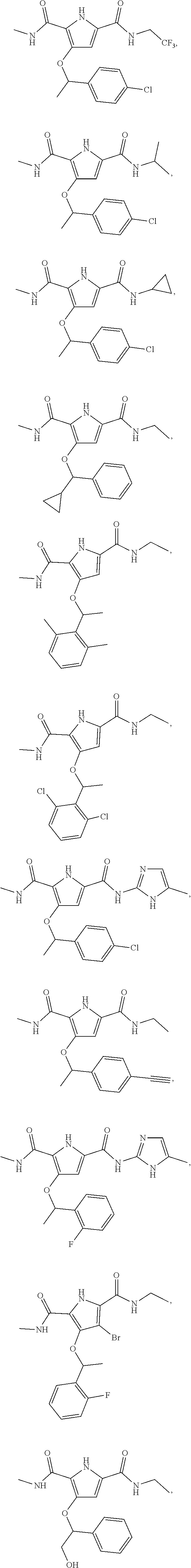

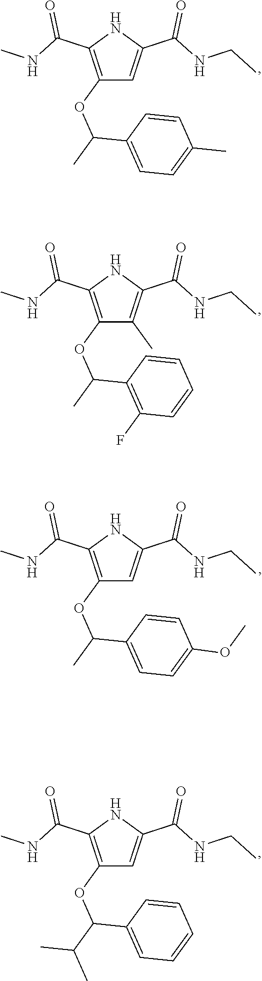

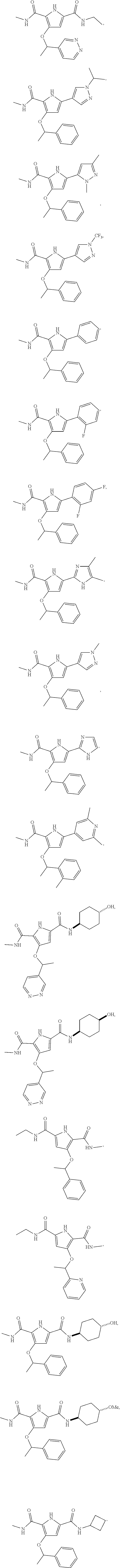

[chemical structure]

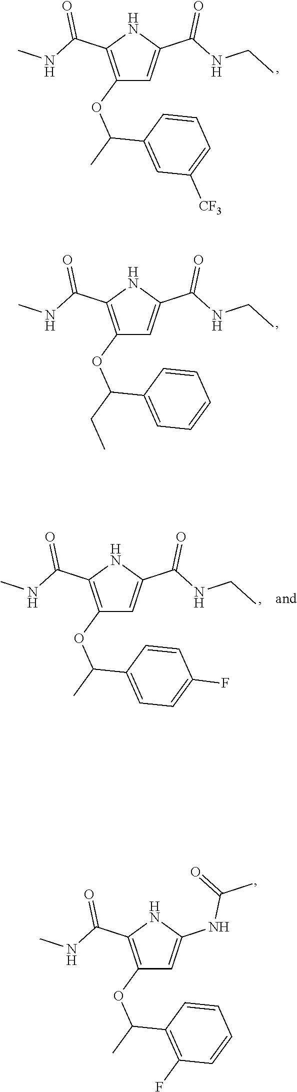

or a stereoisomer or a mixture of stereoisomers thereof.
15. The compound of embodiment 2, or a pharmaceutically acceptable salt or N-oxide thereof, wherein the compound according to formula (IIA) is selected from:

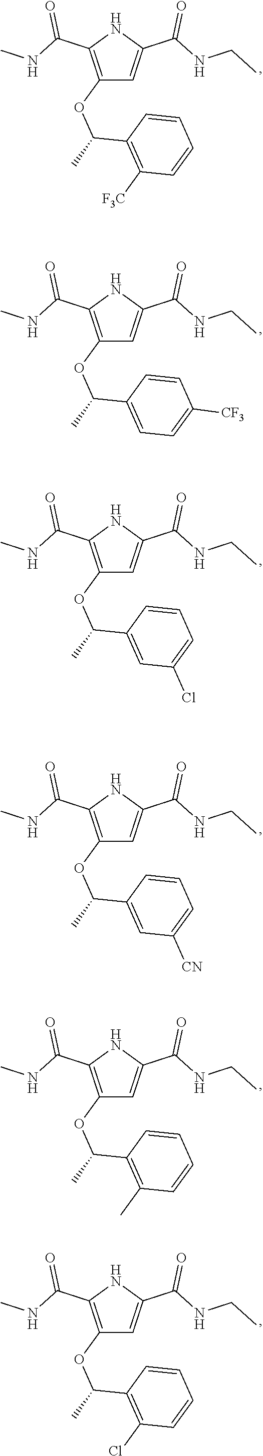

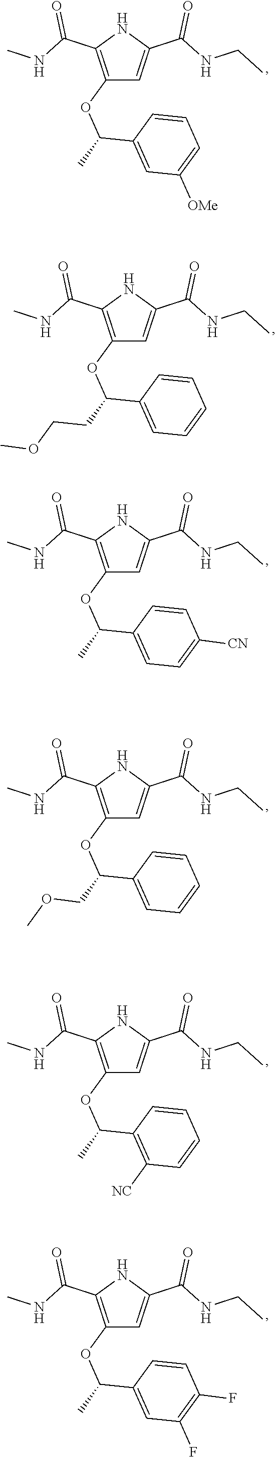

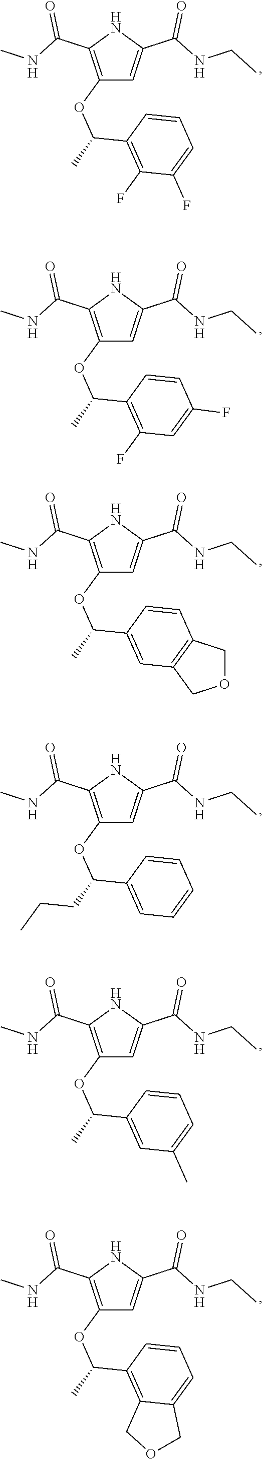

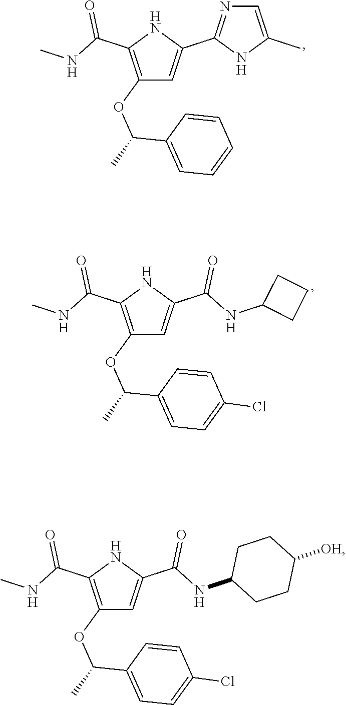

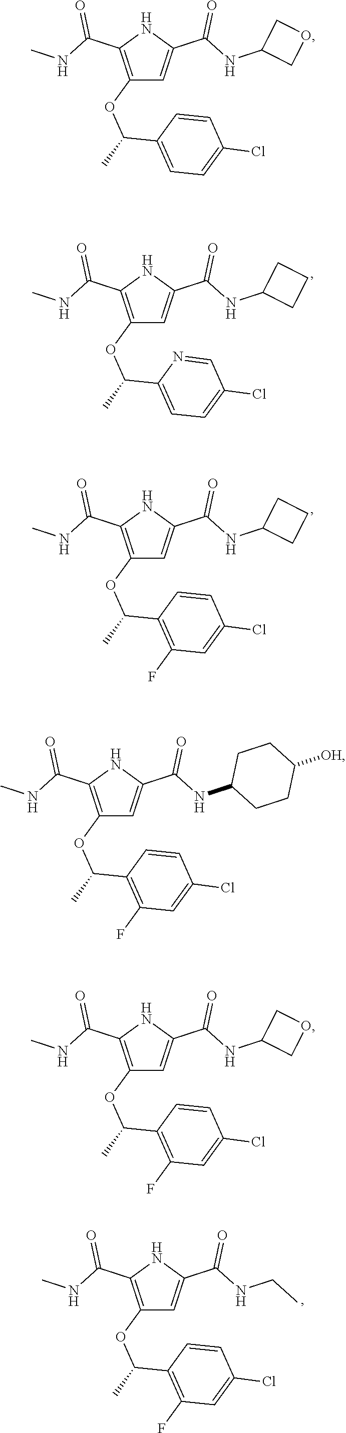

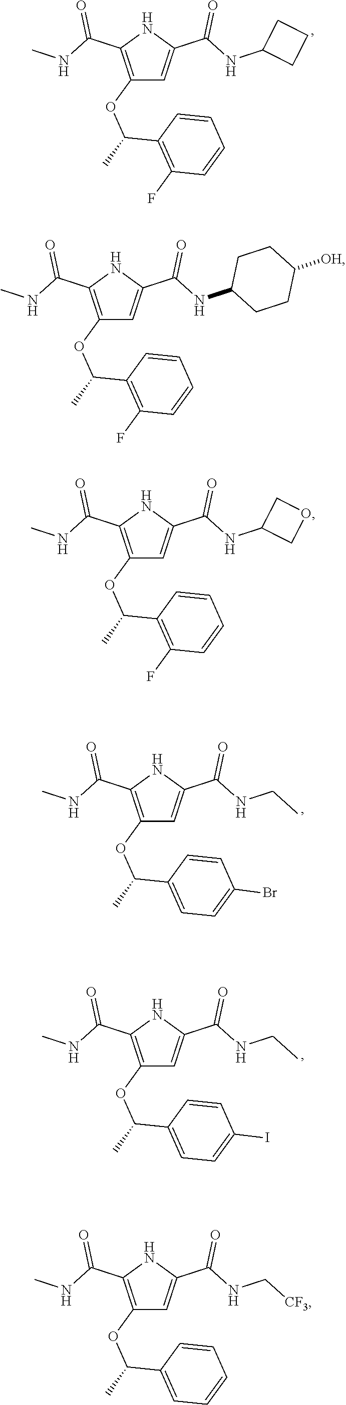

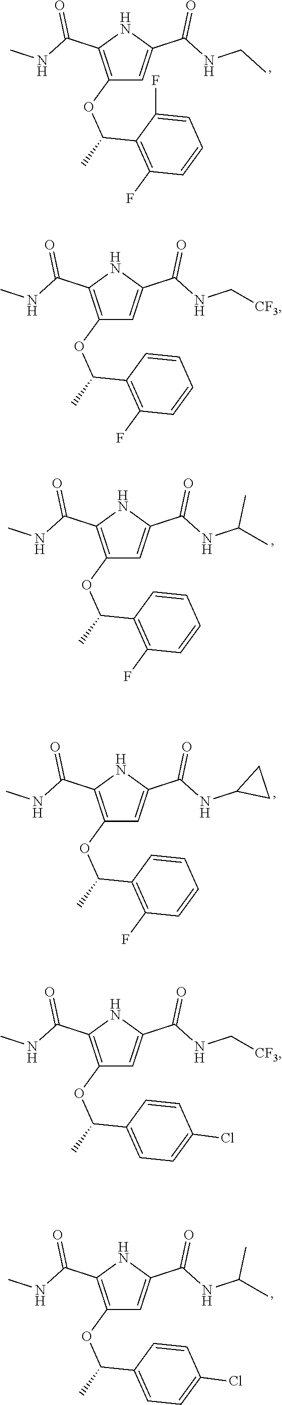

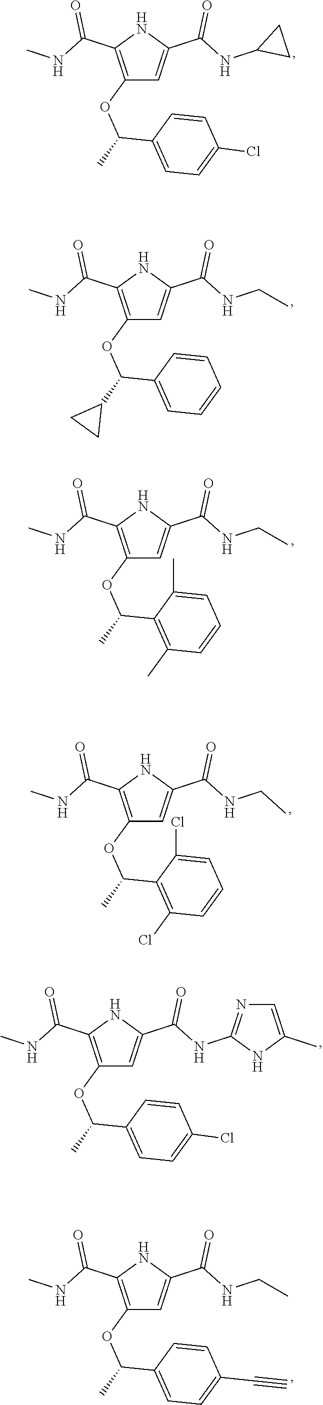

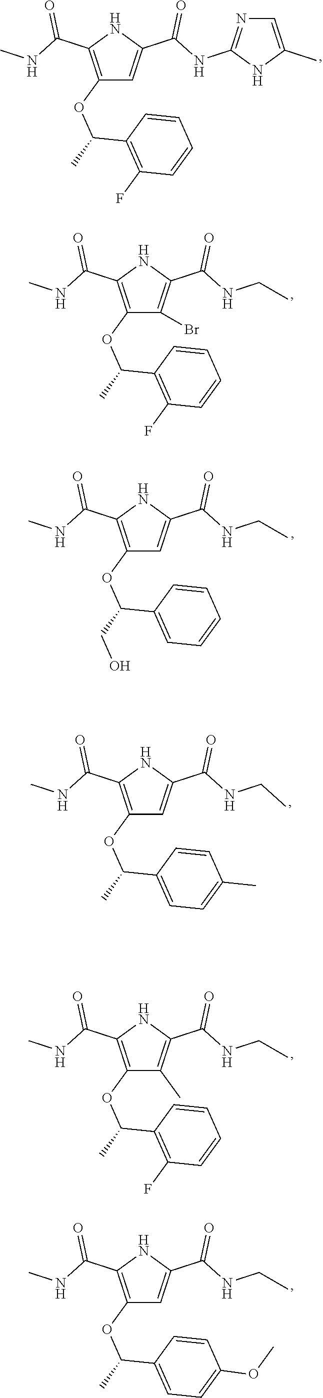

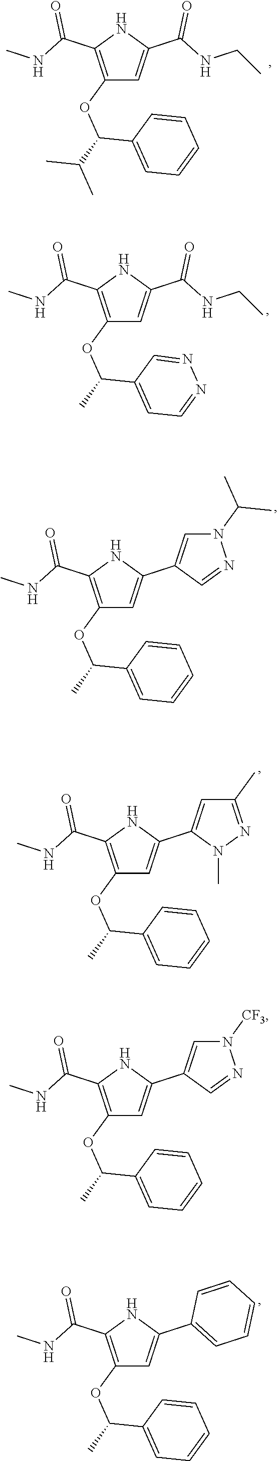

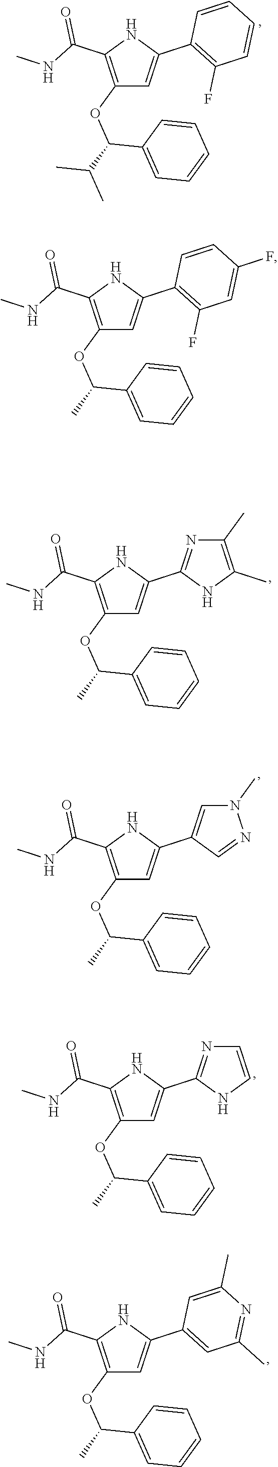

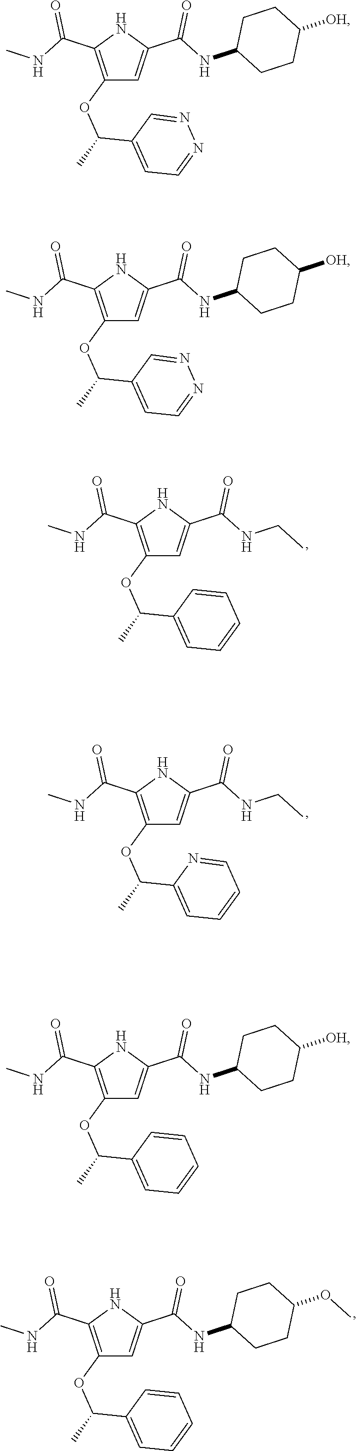

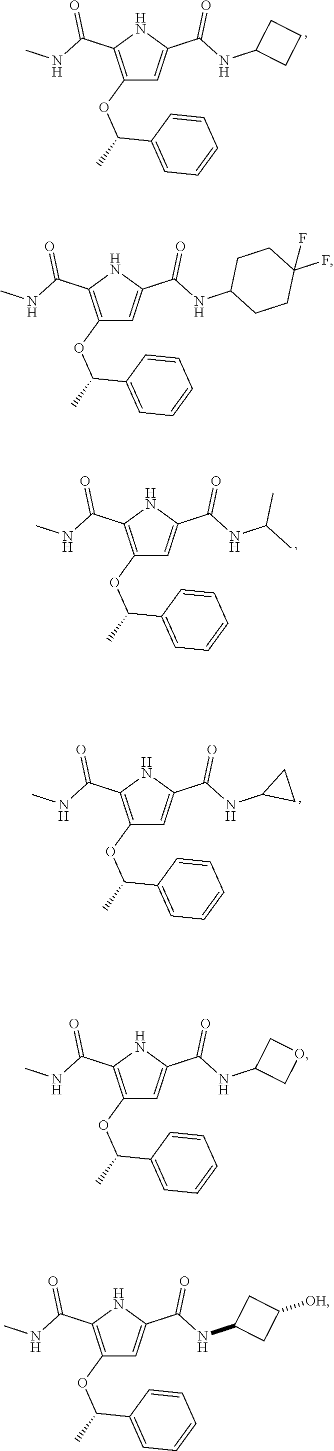

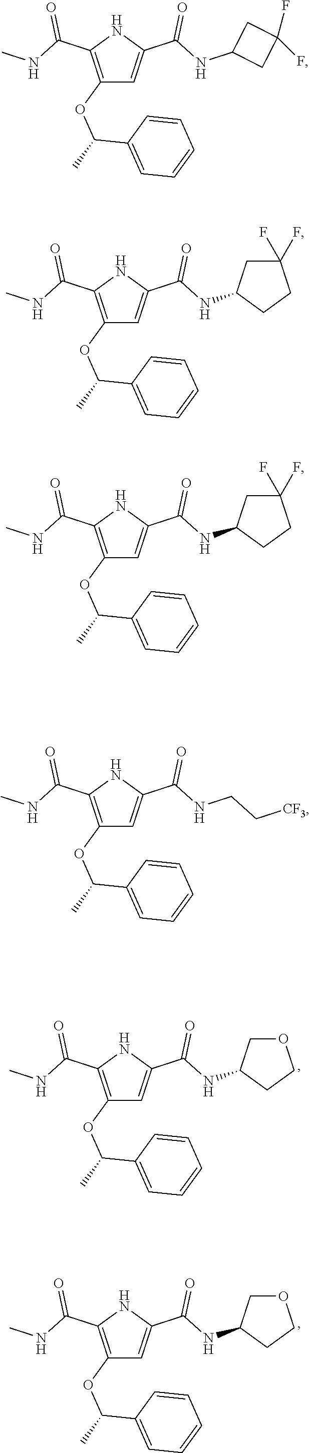 [SMILES: CNC(=O)c1[nH]c(C(=O)NC2CC(F)(F)C2)cc1O[C@@H](C)c1ccccc1 ; CNC(=O)c1[nH]c(C(=O)N[C@H]2CCC(F)(F)C2)cc1O[C@@H](C)c1ccccc1 ; CNC(=O)c1[nH]c(C(=O)N[C@@H]2CCC(F)(F)C2)cc1O[C@@H](C)c1ccccc1 ; CNC(=O)c1[nH]c(C(=O)NCCC(F)(F)F)cc1O[C@@H](C)c1ccccc1 ; CNC(=O)c1[nH]c(C(=O)N[C@H]2CCOC2)cc1O[C@@H](C)c1ccccc1 ; CNC(=O)c1[nH]c(C(=O)N[C@@H]2CCOC2)cc1O[C@@H](C)c1ccccc1]

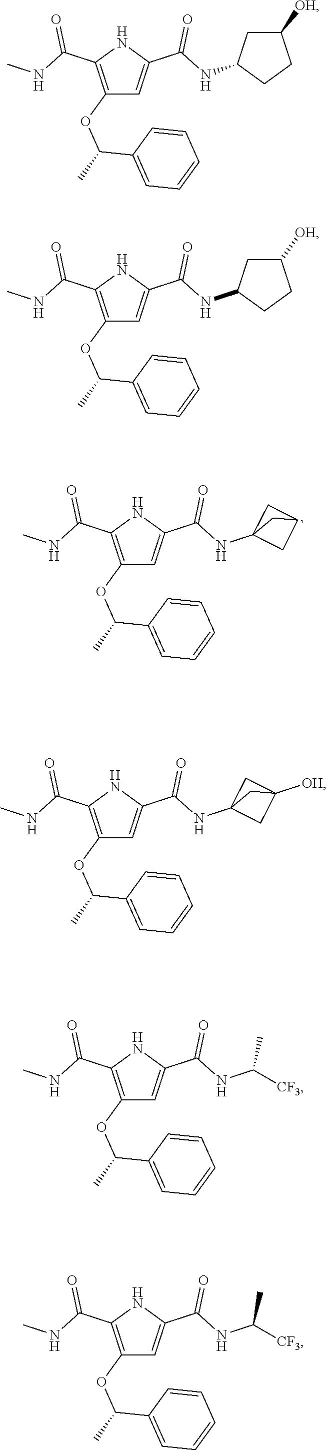

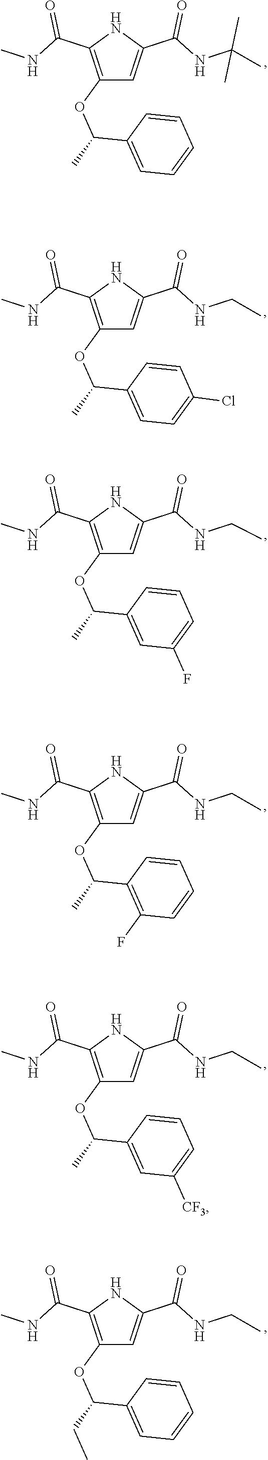

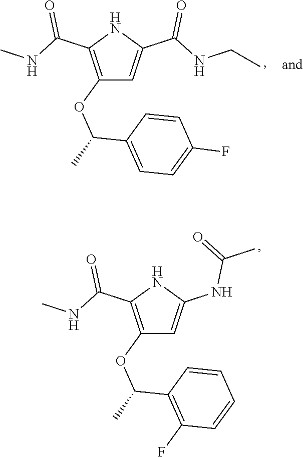

or a stereoisomer thereof.
16. A pharmaceutical composition comprising a compound of any one of embodiments 1 to 15, or a pharmaceutically acceptable salt or N-oxide thereof, and one or more pharmaceutically acceptable excipients.
17. A compound of any one of embodiments 1 to 15, or a pharmaceutically acceptable salt or N-oxide thereof, for use as a medicament.
18. A compound of any one of embodiments 1 to 15, or a pharmaceutically acceptable salt or N-oxide thereof, for use in treating a disease or disorder selected from an inflammatory disorder, an immune disorder, and an autoimmune disorder.
19. A compound of any one of embodiments 1 to 15, or a pharmaceutically acceptable salt or N-oxide thereof, for use in treating a cancer.
20. A compound of any one of embodiments 1 to 15, a tautomer, a stereoisomer or a mixture of stereoisomers, a pharmaceutically acceptable salt, a hydrate, a deuterated derivative, or N-oxide thereof, for use in treating a disease or disorder, wherein the disease or disorder is a joint or joint-related disease or disorder.
21. The compound for use according to embodiment 20, wherein the joint or joint-related disease or disorder is selected from arthritis, bursitis, Ehlers-Danlos syndrome, epicondylitis, Felty Syndrome, gouty arthritis, psoriatic arthritis, osteoarthritis, rheumatoid arthritis, Still's disease, tenosynovitis, synovitis, Sjögren's Syndrome, Lyme disease, Whipple disease, bone cancer, lupus, and other autoimmune joint disorders.
22. The compound for use according to embodiment 20 or 21, wherein the joint or joint-related disease or disorder comprises an arthritis.
23. The compound for use according to embodiment 22, wherein the arthritis comprises rheumatoid arthritis.
24. The compound for use according to any one of embodiments 20 to 23, wherein the disorder is an arthritis and upon administration of a therapeutically effective amount of the compound, tautomer, stereoisomer or mixture of stereoisomers, pharmaceutically acceptable salt, hydrate, deuterated derivative, or N-oxide thereof there is a therapeutic effect associated with reduction in inflammation.
25. The compound for use according to embodiment 24, wherein the therapeutic effect associated with a reduction in inflammation is a reduction in thickness or girth of a joint or limb.
26. The compound for use according to embodiment 24 or 25, wherein there is reduction in arthritic scoring or severity, and
   wherein the reduction in arthritic scoring or severity is a reduction in:
   (a) definite redness and swelling of an ankle/wrist or apparent redness and swelling limited to individual digits, regardless of the number of affected digits;
   (b) severe redness and swelling of an ankle/wrist;
   (c) redness and swelling of the entire appendage including digits; and/or
   (d) maximally inflamed limb with involvement of multiple joints.
27. The compound for use according to any one of embodiments 24 to 26, wherein the reduction is dose dependent.
28. The compound for use according to any one of embodiments 24 to 27, wherein the reduction is by >about 50%, or the reduction is about 50%, or the reduction is between about 25% to about 75%.
29. The compound for use according to any one of embodiments 18 and 20 to 28, wherein the compound, tautomer, stereoisomer or mixture of stereoisomers, pharmaceutically acceptable salt, hydrate, deuterated derivative, or N-oxide thereof is in the form of a pharmaceutical composition which further comprises a pharmaceutically acceptable carrier.
30. The compound for use according to embodiment 29, wherein the compound, tautomer, stereoisomer, pharmaceutically acceptable salt, hydrate, and/or deuterated derivative thereof is formulated as a suspension or partial suspension in the composition.
31. The compound for use according to embodiment 30, wherein the compound, tautomer, stereoisomer or mixture of stereoisomers, pharmaceutically acceptable salt, hydrate, deuterated derivative, or N-oxide thereof is micronized.
32. The compound for use according to embodiment 30 or 31, wherein the compound, tautomer, stereoisomer or mixture of stereoisomers, pharmaceutically acceptable salt, hydrate, deuterated derivative, or N-oxide thereof is in the form of nanoparticles.
33. The compound for use according to any one of embodiments 18 and 20 to 32, wherein the compound, tautomer, stereoisomer or mixture of stereoisomers, pharmaceutically acceptable salt, hydrate, deuterated derivative, or N-oxide thereof is solubilized or partially solubilized in the composition.
34. The compound for use according to of any one of embodiments 18 and 20 to 33, wherein the compound, tautomer, stereoisomer or mixture of stereoisomers, pharmaceutically acceptable salt, hydrate, deuterated derivative, or N-oxide thereof or pharmaceutical composition is administered locally, topically or systemically.
35. The compound for use according to of any one of embodiments 18 and 20 to 34, wherein the compound, tautomer, stereoisomer or mixture of stereoisomers, pharmaceutically acceptable salt, hydrate, deuterated derivative, or N-oxide thereof or pharmaceutical composition is administered orally.
36. The compound for use according to of any one of embodiments 18 and 20 to 35, wherein the compound, tautomer, stereoisomer or mixture of stereoisomers, pharmaceutically acceptable salt, hydrate, deuterated derivative, or N-oxide thereof or pharmaceutical composition has activity against one or more BET domains.
37. A compound of any one of embodiments 1 to 15, a tautomer, a stereoisomer or a mixture of stereoisomers, a pharmaceutically acceptable salt, a hydrate, a deuterated derivative, or N-oxide thereof, for use in the treatment of a joint or joint-related disease in which a therapeutic effect associated with a reduction in inflammation is achieved.
38. A compound of any one of embodiments 1 to 15, a tautomer, a stereoisomer or a mixture of stereoisomers, a pharmaceutically acceptable salt, a hydrate, a deuterated derivative, or N-oxide thereof, for use in the treatment of an arthritic disease in which a therapeutic effect associated with a reduction in inflammation is achieved.
39. A compound of any one of embodiments 1 to 15, a tautomer, a stereoisomer or a mixture of stereoisomers, a pharmaceutically acceptable salt, a hydrate, a deuterated derivative, or N-oxide thereof, for use in the treatment of a fibrotic disease or disorder.
40. The compound for use according to embodiment 39, wherein the disease or disorder is renal fibrosis.
41. The compound for use according to embodiment 39 or 40, wherein, upon administration of a therapeutically effective amount of the compound, tautomer, stereoisomer or mixture of stereoisomers, pharmaceutically acceptable salt, hydrate, deuterated derivative, or N-oxide thereof there is a therapeutic effect associated with a reduction in fibrosis.
42. The compound for use according to embodiment 41, wherein the reduction in fibrosis comprises a reduction in pathology in the kidneys.
43. The compound for use according to embodiment 42, wherein the reduction in pathology in the kidney comprises a reduction in interstitial nephritis, collagen fiber deposition, and nephropathy.
44. The compound for use according to any one of embodiments 41 to 43, wherein the reduction in fibrosis comprises a reduction in inflammatory tissue biomarkers.
45. The compound for use according to embodiment 44, wherein the inflammatory tissue biomarkers include Col1A1, TGF-b1, MCP-1, IL-1b, IL-6, and Timp1.
46. A compound of any one of embodiments 1 to 15, a tautomer, a stereoisomer or a mixture of stereoisomers, a pharmaceutically acceptable salt, a hydrate, a deuterated derivative, or N-oxide thereof, for use in the treatment of a fibrotic disease in which a therapeutic effect associated with a reduction in fibrosis is achieved.
47. A compound of any one of embodiments 1 to 15, a tautomer, a stereoisomer or a mixture of stereoisomers, a pharmaceutically acceptable salt, a hydrate, a deuterated derivative, or N-oxide thereof, for use in the treatment of renal fibrosis in which a therapeutic effect associated with a reduction in fibrosis is achieved.
48. The compound for use of any of embodiments 40 to 47, wherein the progression of fibrosis severity is slowed or retarded.
49. The compound for use of any of embodiments 40 to 47, wherein the appearance or increase of one or more indicators of fibrosis severity is slowed or retarded.

### EXAMPLES

### Experimental Methods for Examples 1-128

### Abbreviations

| | |
|---|---|
| (Prep-)HPLC | (preparative-) High performance liquid chromatography |
| CDI | 1,1′-Carbonyldiimidazole |
| Clᵢₙₜ | Intrinsic clearance |
| C_{Max} | Maximum concentration reached over a time course |
| | experiment |
| DCM | Dichloromethane |
| DEAD | Diethyl azodicarboxylate |
| DEA | Diethylamine |
| DIBAL-H | Diisobutylaluminium hydride |
| DIAD | Diisopropyl azodicarboxylate |
| DIPEA/DIEA | Diisopropylethylamine, Hünig's base |
| DMAP | 4-(Dimethylamino)pyridine |
| DMF | N,N-Dimethylformamide |
| DMPK | Drug metabolism and pharmacokinetics |
| DMSO | Dimethylsulfoxide |
| EDC | N-Ethyl-N′-(3-dimethylaminopropyl)carbodiimide |
| EtOH | Ethanol |
| F | Bioavailability |
| FA | Formic acid |
| FaSSIF | Fasted State Simulated Intestinal Fluid |
| h/hr/hrs | Hours |
| HATU | 1-[Bis(dimethylamino)methylene]-1H-1,2,3-triazolo[4,5- |
| | b]pyridinium 3-oxid hexafluorophosphate |
| Hex | Hexane |
| IPA | Isopropylalcohol |
| LCMS | Liquid chromatography-mass spectrometry |
| m/z | Mass/charge |
| M⁺/M⁻ | Molecular ion |
| MeOH | Methanol |
| MHz | Megahertz |
| min/mins | Minutes |
| NMP | N-methyl-2-pyrrolidone |
| NMR | Nuclear magnetic resonance |
| PBS | Phosphate-buffered saline |
| RP | Reverse phase |
| Rₜ/R_{T}/t_{R} | Retention time |
| RT/rt | Room temperature |
| T_{1/2} | Half-life |
| T3P | Propylphosphonic anhydride |
| V_{dss} | Volume of distribution at steady state |
| % wt | % weight |

### Analytical Methods:

¹H NMR spectra were recorded on Bruker AVANCE III HD 300, Bruker AVANCE NEO 400 or Bruker AVANCE III HD 400 spectrometers. Chemical shifts were denoted in ppm (δ) relative to residual protonated solvent as an internal standard as described in, for example, Gottlieb et al. Journal of Organic Chemistry (1997) 62 7512. The splitting pattern for NMR spectra was denoted as follows: s (singlet), br (broad), d (doublet), t (triplet), m (multiplet) or combinations thereof. Coupling constants (J) were designated in Hz and reported to one decimal place.

Liquid chromatography-mass spectra (LCMS) was recorded using the following systems and running conditions:

| | | | | Initial | Middle | Gradient | Final | Gradient | | Flow |
|---|---|---|---|---|---|---|---|---|---|---|
| | | | | Water | Water | Length | Water | Length | Held | Rate |
| Condition | Instrument | Column | Mobile Phase | (%) | (%) | (min) | (%) | (min) | (min) | (mL/min) |
| A | Shimadzu | HALO 30 | Water 0.1% | 95 | 40 | 1.80 | 0 | 0.20 | 0.70 | 1.50 |
| | LCMS- | mm × 3.0 | FA/CH₃CN 0.1% | | | | | | | |
| | 2020 | mm, 2.6 μm | TFA | | | | | | | |
| B | | | Water | 95 | 30 | 2.00 | 0 | 0.30 | 0.44 | 1.50 |
| | | | 0.1% FA/CH₃CN | | | | | | | |
| | | | 0.1% FA | | | | | | | |
| C | | EVO C18 50 | Water 5 mM | 90 | 30 | 2.00 | 5 | 0.20 | 0.40 | 1.50 |
| D | | mm × 3.0 | NH₄HCO₃)/CH₃CN | 80 | 30 | 1.85 | 5 | 0.25 | 0.50 | 1.50 |
| E | | mm, 2.6 μm | | 90 | 30 | 2.00 | 5 | 2.20 | 0.40 | 1.50 |
| F | | | Water 0.04% | 100 | 40 | 2.20 | 5 | 0.20 | 0.40 | 1.50 |
| G | | | NH₃•H₂O/CH₃CN | 100 | 30 | 1.80 | 0 | 0.20 | 0.70 | 1.50 |
| H | | | Water 0.1% FA/ | 70 | 30 | 1.80 | 0 | 0.20 | 0.70 | 1.50 |
| I | | | MeCN 0.1% FA | 100 | 40 | 2.20 | 0 | 0.30 | 0.30 | 1.50 |
| J | | Polar-C18, 30 | Water 0.1% FA/ | 100 | 0 | 2.00 | — | — | — | 1.50 |
| | | mm × 2.1 | MeCN 0.1% FA | | | | | | | |
| | | mm, 2.6 μm | | | | | | | | |
| 1 | Waters | Waters XBridge | Water 0.1% FA/ | 97 | — | — | 0 | 3.50 | 0.50 | 0.80 |
| | Acquity | (50 mm × 2.1 | 9:1CH₃CN:Water | | | | | | | |
| | UPLC | mm, 2.5 μm) | 0.1% FA | | | | | | | |

Enantiomeric excess (% ee) were determined using the following chiral HPLC systems and running conditions:

| | | | | | Final | Gradient | Flow |
|---|---|---|---|---|---|---|---|
| Run | | | | Initial A | A | Length | Rate |
| Condition | Instrument | Column | Mobile Phase | (%) | (%) | (min) | (mL/min) |
| A | Shimadzu | Cellulose SZ, | A: Hexane | 80 | 80 | 3.0 | 1.67 |
| B | 20ADXR | 0.46 × 5 cm, | (0.1% Et₂NH) | 70 | 70 | 4.0 | 1.67 |
| C | | 3 μm | B: EtOH | 90 | 90 | 3.6 | 1.67 |
| D | | | | 90 | 90 | 6.0 | 1.67 |
| E | | | | 50 | 50 | 4.0 | 1.67 |
| F | | CHIRALPAK | A: Hex (0.1% Et₂NH) | 90 | 90 | 4.0 | 1.67 |
| | | IA-3, 0.46 cm*5 | B: EtOH | | | | |
| | | cm, 3 μm | | | | | |
| G | | CHIRALPAK | A: Hex (0.1% Et₂NH) | 70 | 70 | 8.0 | 1.67 |
| H | | IC-3, 0.46 cm*5 | B: EtOH | 80 | 80 | 6.0 | 1.00 |
| | | cm, 3 μm | | | | | |
| I | | Cellulose SB | A: Hex (0.1% DEA) | 70 | 70 | 10 | 1.67 |
| | | 0.46 cm × 10 | B: IPA | | | | |
| | | cm, 3 μm | | | | | |
| K | | Cellulose SB | A: Hex (0.1% DEA) | 90 | 90 | 4 | 1.67 |
| L | | 46 × 50 mm, | B: EtOH | 70 | 70 | 4 | 1.67 |
| | | 3 μm | | | | | |
| M | | Lux Cellulose-2. | A: Hex (0.1% DEA) | 90 | 90 | 10 | 1.67 |
| | | 4.6*50 mm, | B: EtOH | | | | |
| | | 3 μm | | | | | |
| N | Agilent-SFC | Cellulose SB | A: CO₂ | 90 | 50 | 4 (+2 hold) | 2.00 |
| | 1260 | 46 × 50 mm, | B: MeOH | | | | |
| | | 3 μm | (20 mM NH₃) | | | | |
| O | | CHIRALPAK IC-3, | A: CO₂ | 95 | 80 | 4 (+2 hold) | 2.00 |
| | | 3.0*100 mm, | B: IPA | | | | |
| | | 3 μm | (20 mM NH₃) | | | | |
| P | | CHIRALPAK IA-3, | A: CO₂ | 90 | 50 | 4 (+2 hold) | 2.00 |
| | | 3.0*100 mm, | B: MeOH | | | | |
| | | 3 μm | (0.1% DEA) | | | | |

| | | | | Initial | Final | Gradient | Flow | Back | |
|---|---|---|---|---|---|---|---|---|---|
| Run | | | | A | A | Length | Rate | Pressure | Temp |
| Condition | Instrument | Column | Mobile Phase | (%) | (%) | (min) | (mL/min) | (Bar) | (° C.) |
| 1 | SFC | CHIRALPAK IG | A: Liq CO₂ | 5 | 50 | 5 | 4 | 100 | 40 |
| | Investigator | (250 × 4.6 mm | B: 0.1% Methanolic | | | | | | |
| | with PDA | 5 μm) | Ammonia in 2- | | | | | | |
| | Detector | | propanol:Acetonitrile | | | | | | |
| | | | (70:30) | | | | | | |
| 2 | | | A: Liq CO₂ | 5 | 50 | 5 | 4 | 100 | 40 |
| | | | B: 0.1% Methanolic | | | | | | |
| | | | Ammonia in | | | | | | |
| | | | Methanol:Acetonitrile | | | | | | |
| | | | (50:50) | | | | | | |
| 3 | | | A: Liq CO₂ | 50 | 50 | 10 | 4 | 100 | 40 |
| 4 | | | B: 0.1% Methanolic | 55 | 45 | 5 | 4 | 100 | 40 |
| 5 | | | Ammonia in | 60 | 40 | 10 | 4 | 100 | 40 |
| 6 | | | Methanol:Acetonitrile | 65 | 35 | 10 | 4 | 100 | 40 |
| | | | (50:50) | | | | | | |
| 7 | | | A: Liq CO₂ | 60 | 40 | 10 | 4 | 100 | 40 |
| | | | B: Methanol | | | | | | |
| 8 | | CHIRALPAK IB-N | A: Liq CO₂ | 5 | 50 | 5 | 4 | 100 | 40 |
| | | (250 × 4.6 mm | B: 0.1% Methanolic | | | | | | |
| | | 5 μm) | Ammonia in | | | | | | |
| | | | Methanol:Acetonitrile | | | | | | |
| | | | (50:50) | | | | | | |
| 9 | Shimadzu | CHIRALPAK IG | A: Methanol | 100 | 0 | 10 | 1 | NA | NA |
| | LC-20 AD | (250 × 4.6 mm | B: Acetonitrile | | | | | | |
| | system | 5 μm) | | | | | | | |
| 10 | with DAD | CHIRALPAK IB-N | A: 0.1% M•NH₃ | 80 | 20 | 20 | 1 | NA | NA |
| | detector | (250 × 4.6 mm | in n-Heptane | | | | | | |
| | | 5 μm) | B: 0.1% M•NH₃ | | | | | | |
| | | | in 2-Propanol- | | | | | | |
| | | | Methanol (50-50) | | | | | | |
| 11 | | YMC CELLULOSE SC | A: 0.1% M•NH₃ | 50 | 50 | 30 | 1 | NA | NA |
| | | (250 × 4.6 mm | in n-Heptane | | | | | | |
| | | 5 μm) | B: 0.1% M•NH₃ | | | | | | |
| | | | in 2-Propanol | | | | | | |

### Purification Methods:

Purification by preparative HPLC (prep-HPLC) employed the following instruments and conditions:

| | | | | Initial | Final | Flow | Gradient |
|---|---|---|---|---|---|---|---|
| Run | | | | Water | Water | Rate | Length |
| Condition | Instrument | Column | Mobile Phase | (%) | (%) | (mL/min) | (min) |
| A | Waters | Xselect CSH | Water | 95 | 60 | 60 | 9 |
| | 2545 Binary | C18 OBD Column | (0.1% FA)/MeOH | | | | |
| | Gradient | 30*150 mm | | | | | |
| | Module with | 5 μm | | | | | |
| B | Waters 2489 | XBridge Prep | Water (10 mM | 82 | 50 | 60 | 8 |
| | detector | OBD C18 Column, | NH₄HCO₃)/MeCN | | | | |
| | | 30*150 mm, | | | | | |
| | | 5 μm | | | | | |
| C | | YMC-Actus | Water (10 mM | 75 | 55 | 60 | 7 |
| | | Triart C18, | NH₄HCO₃)/MeCN | | | | |
| | | 30 × 150 mm, | | | | | |
| | | 5 μm | | | | | |
| D | | XBridge Prep | Water (10 mM | 69 | 50 | 60 | 7 |
| | | OBD C18 Column, | NH₄HCO₃)/MeCN | | | | |
| | | 30 × 150 mm, | | | | | |
| | | 5 μm | | | | | |
| E | | XBridge Prep | Water (10 mM | 65 | 45 | 60 | 7 |
| | | OBD C18, | NH₄HCO₃)/MeCN | | | | |
| | | 30 × 150 mm, | | | | | |
| | | 5 μm | | | | | |
| F | | YMC-Actus | Water (10 mM | 59 | 42 | 60 | 7 |
| | | Triart C18 ExRS, | NH₄HCO₃)/MeCN | | | | |
| | | 30 × 150 mm, | | | | | |
| | | 5 μm | | | | | |
| H | Biotage | C18 | Water (0.1% | 90 | 50 | 60 | 10 |
| | Isolera | | FA)/MeCN | | | | |
| I | Prime | | Water (0.1% | 100 | 50 | 60 | 50 |
| | | | NH3•H₂O)/MeCN | | | | |

Preparative chiral HPLC purifications were conducted using the following systems and running conditions:

| | | | | Initial | Final | Gradient | Flow |
|---|---|---|---|---|---|---|---|
| Run | | | | A | A | Length | Rate |
| Condition | Instrument | Column | Mobile Phase | (%) | (%) | (min) | (mL/min) |
| A | Agela Octopus | CHIRAL ART | | 80 | 80 | 15.0 | 45.0 |
| B | | Cellulose-SJ, | A: Hexane | 70 | 70 | 9.5 | 45.0 |
| | | 3 × 25 cm, | (0.5% 2M NH₃ | | | | |
| | | 5 μm | in MeOH) | | | | |
| | | | B: EtOH | | | | |
| C | | Amylose-C NEO, | A: Hexane | 90 | 90 | 35.0 | 40.0 |
| | | 3 × 25 cm, | (10 mM NH₃ | | | | |
| | | 5 μm | in MeOH) | | | | |
| | | | B: Isopropanol | | | | |
| D | | CHIRAL ART | A: Hexane | 90 | 90 | 30.0 | 20.0 |
| | | Cellulose-SB, | (10 mM NH₃ | | | | |
| | | 2 × 25 cm, | in MeOH) | | | | |
| | | 5 μm | B: EtOH | | | | |
| E | Gilson 281 | CHIRAL ART | A: Hex (10 mM | 70 | 70 | 20 | 20 |
| | | Cellulose-SB, | NH₃—MeOH) | | | | |
| | | 2*25 cm, | B: CH₃CN (0.1% | | | | |
| | | 5 μm | Isopropylamine) | | | | |
| F | | | A: Hex (10 mM | 90 | 90 | 30 | 20 |
| | | | NH₃—MeOH), | | | | |
| | | | B: EtOH | | | | |
| G | | | A: Hex (10 mM | 90 | 90 | 20 | 20 |
| | | | NH₃—MeOH) | | | | |
| | | | B: CH₃CN:EtOH = 2:1 | | | | |
| H | | | A: Hex | 70 | 70 | 20 | 20 |
| | | | (0.5% 2M NH₃—MeOH), | | | | |
| | | | B: IPA | | | | |
| I | | CHIRAL ART | A: Hex | 70 | 70 | 30 | 40 |
| | | Cellulose-SB, | (0.5% 2M NH₃—MeOH), | | | | |
| | | 3*25 cm, | B: EtOH | | | | |
| | | 5 μm | | | | | |
| J | | CHIRAL ART | A: Hex | 50 | 50 | 10 | 20 |
| | | Cellulose-SZ, | (10 mM NH₃—MeOH), | | | | |
| K | | 2.0*25 cm, | B: EtOH | 50 | 50 | 20 | 20 |
| | | 5 μm | | | | | |
| M | | Lux 5 um | A: Hex | 90 | 90 | 20 | 20 |
| | | Cellulose-2 | (0.5% 2M NH₃—MeOH) | | | | |
| | | 2.12*25 cm, | B: EtOH | | | | |
| | | 5 μm | | | | | |
| N | | CHIRALPAKIC | A: Hex | 70 | 70 | 19 | 140 |
| | | 5*25 cm, | (10 mM NH₃—MeOH) | | | | |
| | | 5 μm | B: IPA | | | | |
| O | | CHIRALPAKIC | A: Hex | 70 | 70 | 10 | 20 |
| | | 2*25 cm, | (10 mM NH₃—MeOH) | | | | |
| | | 5 μm | B: EtOH | | | | |
| P | | CHIRAL ART | A: Hex | 90 | 90 | 20 | 20 |
| | | Amylose-SA | (10 mM NH₃—MeOH) | | | | |
| | | 2*25 cm, | B: ACN:EtOH = 2:1 | | | | |
| | | 5 μm | | | | | |
| Q | Waters Prep- | CHIRAL ART | A: CO₂ | 60 | 60 | 20 | 100 |
| | SFC-350-01 | Cellulose-SC | B: IPA | | | | |
| | | 3*25 cm, | | | | | |
| | | 5 μm | | | | | |
| R | | CHIRAL ART | A: CO₂ | 65 | 65 | 20 | 150 |
| | | Amylose-SA | B: MeOH | | | | |
| | | 5*25 cm, | (0.1% 2M NH₃—MeOH) | | | | |
| | | 5 μm | | | | | |
| S | | CHIRAL ART | A: CO₂ | 75 | 75 | 10 | 100 |
| | | Cellulose-SB, | B: MeOH | | | | |
| | | 3*25 cm, | | | | | |
| | | 5 μm | | | | | |
| 1 | SHIMADZU | CHIRALPAK IG | A: Methanol | 100 | 0 | 30 | 40 |
| 2 | LC20AP Prep | (250 × 50 mm | B: Acetonitrile | 70 | 30 | 30 | 60 |
| 3 | HPLC with UV | 5 μm) | | 90 | 10 | 30 | 50 |
| 4 | Detector | | A: 0.1% Methanolic | 80 | 20 | 30 | 60 |
| 5 | | | Ammonia Methanol | 100 | 0 | 30 | 27 |
| 6 | | | B: 0.1% Methanolic | 100 | 0 | 30 | 25 |
| 7 | | | Ammonia Acetonitrile | 100 | 0 | 30 | 50 |
| 8 | | | | 100 | 0 | 30 | 30 |
| 9 | Agilent 1260 | CHIRALPAK IB-N | A: 0.1% Methanolic | 93 | 7 | 30 | 15 |
| | Infinity Prep | (250 × 10 mm | Ammonia in n-Heptane | | | | |
| | HPLC with UV | 5 μm) | B: 0.1% Methanolic | | | | |
| | Detector | | Ammonia in 2- | | | | |
| | | | Propanol:Acetonitrile | | | | |
| | | | (70:30) | | | | |
| 10 | | | A: 0.1% Methanolic | 90 | 10 | 30 | 15 |
| | | | Ammonia in n-Heptane | | | | |
| | | | B: 0.1% Methanolic | | | | |
| | | | Ammonia in IPA | | | | |
| 11 | | YMC Cellulose SC | A: 0.1% Methanolic | 80 | 20 | 30 | 20 |
| | | (250 × 20 mm | Ammonia in n-Heptane | | | | |
| | | 5 μm) | B: 0.1% Methanolic | | | | |
| | | | Ammonia in 2- | | | | |
| | | | Propanol:Acetonitrile | | | | |
| | | | (70:30) | | | | |
| 12 | | | A: 0.1% Methanolic | 70 | 30 | 30 | 20 |
| | | | Ammonia in n-Heptane | | | | |
| | | | B: 0.1% Methanolic | | | | |
| | | | Ammonia in IPA | | | | |
| 13 | Waters PSFC | CHIRALPAK IG | A: Liq CO2 | 50 | 50 | 20 | 150 |
| 14 | 350 with UV | (250 × 50 mm | B: 0.1% Methanolic | 60 | 40 | 20 | 150 |
| 15 | Detector | 5 μm) | Ammonia in | 70 | 30 | 35 | 150 |
| 16 | | | Methanol:Acetonitrile | 60 | 40 | 20 | 160 |
| | | | (50:50) | | | | |
| 17 | | | A: Liq Co2 | 80 | 20 | 20 | 150 |
| | | | B: 0.1% Methanolic | | | | |
| | | | Ammonia in 2- | | | | |
| | | | Propanol:Acetonitrile | | | | |
| | | | (70:30) | | | | |

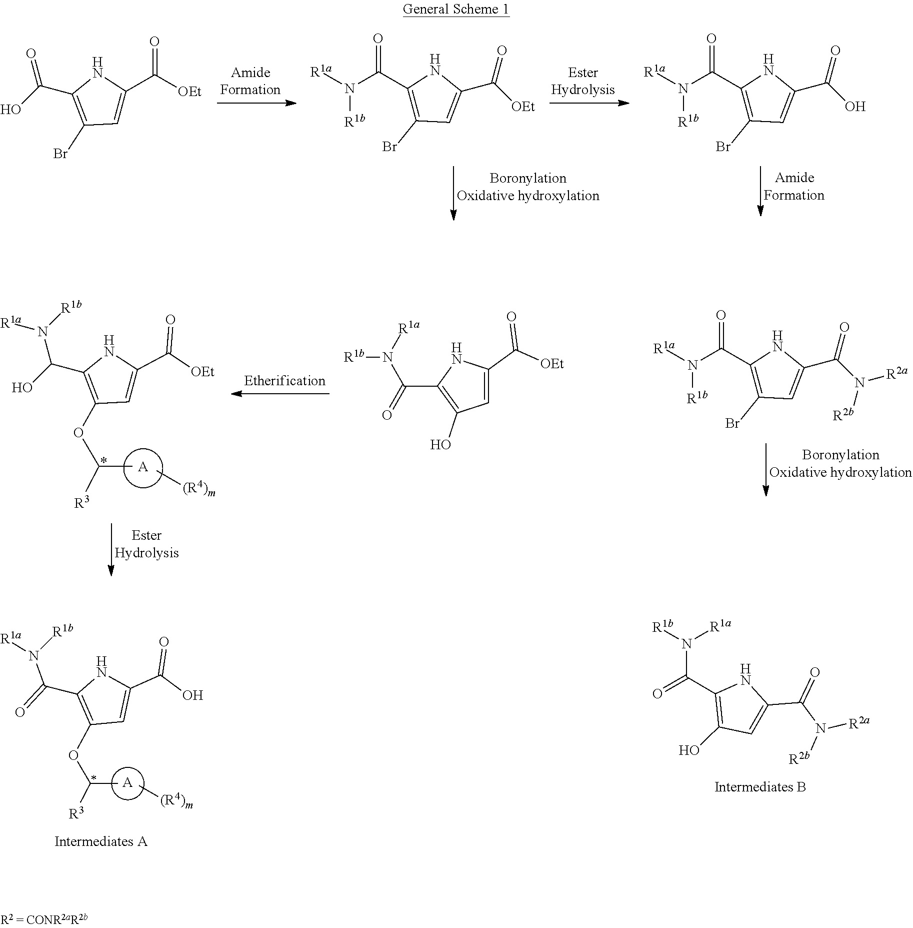

Intermediates A and B may be synthesised according to the route illustrated in General Scheme 1 and described in the following examples. The synthesis of starting material 3-bromo-5-(ethoxycarbonyl)-1H-pyrrole-2-carboxylic acid can be achieved according to method described in, for example, CN106187854A and is described in the following examples.

Amide formation may be achieved using a variety of conditions known to those skilled in the art, for example, by activating the carboxylic acid with common coupling reagents (for example EDC, HATU, T3P, CDI) prior to reaction with the corresponding amine with an appropriate base (for example triethylamine, DIPEA, DMAP) in an appropriate solvent (for example DCM, DMF, NMP, THF, ethyl acetate) with heating if necessary. Alternatively, the carboxylic acid may first be converted to an acyl halide (using for example thionyl chloride) prior to reaction with the corresponding amine with an appropriate base (for example triethylamine, DIPEA, DMAP) in an appropriate solvent (for example DCM, DMF, NMP, THF, 1,4-dioxane) with heating if required.

Etherification may be conducted using a variety of conditions known to those skilled in the art, for example, by alkylation using an appropriate alky halide, mesylate or tosylate in the presence of a suitable base (for example triethylamine, diisopropylethylamine, potassium carbonate, potassium tert-butoxide, lithium bis(trimethylsilyl)amide or sodium hydride), in a suitable solvent (for example THF, 1,4-dioxane, diethylether, DMF, NMP) with heating if required. Alternatively, etherification may be achieved using Mitsunobu conditions reacting the appropriate alcohol in the presence of a coupling agent (for example DEAD, DIAD) in a suitable solvent (for example THF, 1,4-dioxane) with heating if required.

Boronylation and oxidative hydroxylation can be achieved in a two-step process. First, the bromide is converted to a boronic acid or ester using a variety of conditions known to those skilled in the art, for example, by use of a suitable boron source (for example bis(pinacolato)diboron) in the presence of a suitable catalyst (for example XPhos Pd G3, palladium tetrakis, Pd(dppf)Cl₂), using a suitable base (for example Na₂CO₃, Cs₂CO₃, KOAc) in a suitable solvent (for example 1,4-doxane, THF) with heating if required. Second, oxidative hydroxylation is achieved by use of a suitable base, if necessary (for example sodium hydroxide) in the presence of a suitable oxidising agent (for example hydrogen peroxide, Oxone) in the presence of a suitable solvent (for example water), with heating if required.

Ester hydrolysis may be achieved using a variety of conditions known to those skilled in the art, for example, by use of an appropriate base (for example lithium hydroxide, sodium hydroxide, potassium hydroxide) in a suitable solvent (for example water, THF, 1,4-dioxane, methanol, ethanol or mixtures thereof) with heating if required.

It will be appreciated by those skilled in the art that Intermediates A may be isolated as a racemate around the potential chiral centre (*). Alternatively, the enantiomers (if applicable) can be separated using techniques well known to those skilled in the art (for example chiral chromatography). Alternatively, use of the appropriate enantiopure starting materials may yield Intermediates A as a single enantiomer.

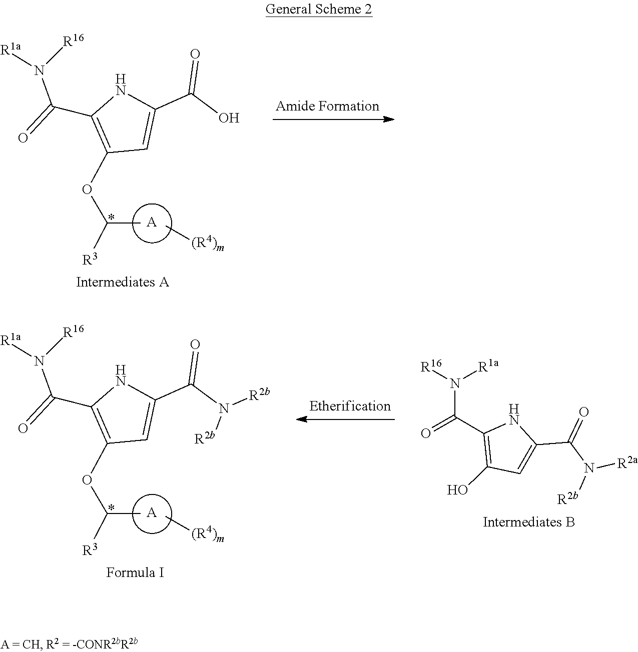

A sub-set of compounds of Formula I may be synthesised from Intermediates A and B according to the route illustrated in General Scheme 2 and described in the following examples.

Amide formation may be achieved using a variety of conditions known to those skilled in the art, for example, by activating the carboxylic acid with common coupling reagents (for example EDC, HATU, T3P, CDI) prior to reaction with the corresponding amine with an appropriate base (for example triethylamine, DIPEA, DMAP) in an appropriate solvent (for example DCM, DMF, NMP, THF, ethyl acetate) with heating if necessary. Alternatively, the carboxylic acid may first be converted to an acyl halide (using for example thionyl chloride) prior to reaction with the corresponding amine with an appropriate base (for example triethylamine, DIPEA, DMAP) in an appropriate solvent (for example DCM, DMF, NMP, THF, 1,4-dioxane) with heating if required.

Etherification may be conducted using a variety of conditions known to those skilled in the art, for example, by alkylation using an appropriate alky halide, mesylate or tosylate in the presence of a suitable base (for example triethylamine, diisopropylethylamine, potassium carbonate, potassium tert-butoxide, lithium bis(trimethylsilyl)amide or sodium hydride), in a suitable solvent (for example THF, 1,4-dioxane, diethylether, DMF, NMP) with heating if required. Alternatively, etherification may be achieved using Mitsunobu conditions reacting the appropriate alcohol in the presence of a coupling agent (for example DEAD, DIAD) in a suitable solvent (for example THF, 1,4-dioxane) with heating if required.

It will be appreciated by those skilled in the art that compounds of Formula I may be isolated as a racemate around the potential chiral centre (*). Alternatively, the enantiomers (if applicable) can be separated using techniques well known to those skilled in the art (for example chiral chromatography). Alternatively, use of the appropriate enantiopure starting materials may yield compounds of Formula I as a single enantiomer. The compounds of the disclosure may be obtained according to or analogously to the methods described in General Schemes 1 and 2 above. Set out in Examples 1 to 128 below are various methods of preparation of the compounds including their purification, isolation, and separation. The compounds of the disclosure may be obtained according to or analogously to the methods described in Examples 1 to 128 below.

### Example 1—Racemic N⁵-ethyl-N²-methyl-3-(1-(4-(trifluoromethyl) phenyl) ethoxy)-1H-pyrrole-2,5-dicarboxamide

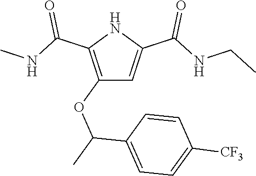 [SMILES: CCNC(=O)c1cc(OC(C)c2ccc(C(F)(F)F)cc2)c(C(=O)NC)[nH]1]

### Preparation 1: 1-(1-bromoethyl)-4-(trifluoromethyl)benzene

1-(4-(trifluoromethyl) phenyl) ethan-1-ol (2.0 g, 10.5 mmol) was dissolved in DCM (40 mL) under nitrogen. Phosphorous tribromide (4.5 g, 16.8 mmol) was dropwise added to the reaction mixture at 0° C. The reaction was stirred at room temperature for 16 h. The resulting suspension was diluted with water (50 mL) and extracted with ethyl acetate (2×50 mL). The organic layer was washed with brine solution (2×50 mL) and concentrated under vacuum to afford pure material as yellow liquid. (2.0 g, 75%). ¹H NMR: (400 MHz, DMSO) δ 7.74-7.68 (m, 4H), 5.57 (q, J=6.8 Hz, 1H), 1.99 (d, J=7.2 Hz, 3H)

### Preparation 2: N⁵-ethyl-N²-methyl-3-(1-(4-(trifluoromethyl) phenyl) ethoxy)-1H-pyrrole-2,5-dicarboxamide

N⁵-ethyl-3-hydroxy-N²-methyl-1H-pyrrole-2,5-dicarboxamide (0.10 g, 0.47 mmol) was dissolved in THE (1 mL) under nitrogen. Potassium tert-butoxide (0.085 g, 0.75 mmol) and 18-crown-6 (0.012 g, 0.047 mmol) were added to the reaction mixture followed by 1-(1-bromoethyl)-4-(trifluoromethyl) benzene (0.23 g, 0.94 mmol) at room temperature. The reaction was stirred at room temperature for 16 h. The resulting suspension was diluted with water (25 mL) and extracted with ethyl acetate (2×25 mL). The organic layer was washed with brine solution (2×25 mL) and concentrated under vacuum to afford crude material. The crude material was purified by flash chromatography in reverse phase using Biotage select with C18 silica 50 μm with product eluted in (50:50) acetonitrile/water) to give titled product as an off-white solid (0.013 g, 7%). ¹H NMR: (400 MHz, DMSO) δ 11.28 (s, 1H), 8.22 (t, J=4.8 Hz, 1H), 7.74-7.67 (m, 4H), 7.26-7.24 (m, 1H), 6.32 (s, 1H), 5.51-5.49 (m, 1H), 3.17 (q, J=2.8 Hz, 2H), 2.86 (d, J=4.4 Hz, 3H), 1.61 (d, J=6.4 Hz, 3H), 1.05 (t, J=7.2 Hz, 3H). LCMS¹: m/z=384 [M+H]⁺.

### Example 2—Racemic N⁵-ethyl-N²-methyl-3-(1-(2-(trifluoromethyl) phenyl) ethoxy)-1H-pyrrole-2,5-dicarboxamide

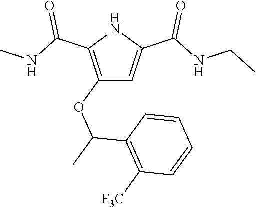 [SMILES: CCNC(=O)c1cc(OC(C)c2ccccc2C(F)(F)F)c(C(=O)NC)[nH]1]

Following the procedure in Example 1, Preparation 2, using 1-(1-bromoethyl)-2-(trifluoromethyl) benzene to give title compound as off-white solid (0.031 g, 17%). ¹H NMR: (400 MHz, DMSO) δ 11.35 (s, 1H), 8.22 (t, J=4.8 Hz, 1H), 7.85 (d, J=7.6 Hz, 1H), 7.75-7.69 (m, 2H), 7.51 (apparent t, J=7.6 Hz, 1H), 7.30-7.29 (m, 1H), 6.13 (s, 1H), 5.55 (q, J=5.6 Hz, 1H), 3.18-3.12 (m, 2H), 2.87 (d, J=4.8 Hz, 3H), 1.64 (d, J=6.4, 3H), 1.05 (t, J=7.2 Hz, 3H). LCMS¹: m/z=384 [M+H]⁺.

### Example 3—Racemic 3-(1-(3-chlorophenyl) ethoxy)-N⁵-ethyl-N²-methyl-1H-pyrrole-2, 5-dicarboxamide

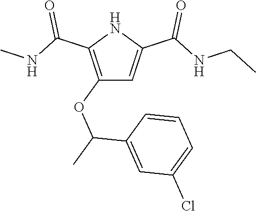 [SMILES: CCNC(=O)c1cc(OC(C)c2cccc(Cl)c2)c(C(=O)NC)[nH]1]

### Preparation 3: 1-(1-bromoethyl)-3-chlorobenzene

Following the procedure in Example 1, Preparation 1, 1-(3-chlorophenyl)ethan-1-ol (2 g, 12.8 mmol) was reacted to give title compound as off-white liquid (1.8 g, 64%). ¹H NMR: (400 MHz, DMSO) δ 7.59-7.36 (m, 4H), 5.49 (q, J=7.2 Hz, 1H), 1.97 (d, J=6.8 Hz, 3H).

### Preparation 4: 3-(1-(3-chlorophenyl) ethoxy)-N⁵-ethyl-N²-methyl-1H-pyrrole-2, 5-dicarboxamide

Following the procedure in Example 1, Preparation 2, using 1-(1-bromoethyl)-3-chlorobenzene to give title compound as off-white solid (0.030 g, 26%). ¹H NMR: (400 MHz, DMSO) δ 11.27 (br s, 1H), 8.23 (br s, 1H), 7.56 (s, 1H), 7.43-7.25 (m, 4H), 6.34 (s, 1H), 5.40-5.38 (m, 1H), 3.17 (br s, 2H), 2.85 (d, J=4.4 Hz, 3H), 1.59 (d, J=6.4 Hz, 3H), 1.06 (t, J=7.2 Hz, 3H). LCMS¹: m/z=350 [M+H]⁺.

### Example 4—3-(1-(3-cyanophenyl) ethoxy)-N⁵-ethyl-N²-methyl-1H-pyrrole-2,5-dicarboxamide

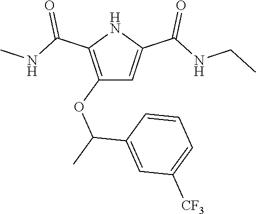 [SMILES: CCNC(=O)c1cc(OC(C)c2cccc(C(F)(F)F)c2)c(C(=O)NC)[nH]1]

### Preparation 5: 3-(1-hydroxyethyl)benzonitrile

Following the procedure in Example 10, Preparation 19, using 3-acetylbenzonitrile to give title compound as colourless oil (1.3 g, 64%). ¹H NMR: (400 MHz, DMSO) δ 7.77 (s, 1H), 7.70 (q, J=6.8 Hz, 2H), 7.53 (t, J=8.0 Hz, 1H), 5.40 (d, J=4.4 Hz, 1H), 4.81-4.75 (m, 1H), 1.33 (d, J=6.4 Hz, 3H).

### Preparation 6: 3-(1-bromoethyl)benzonitrile

Following the procedure in Example 1, Preparation 1, using 3-(1-hydroxyethyl)benzonitrile to give title compound colourless oil (1.3 g, 76%). ¹H NMR: (400 MHz, DMSO) δ 8.02 (t, J=1.6 Hz, 1H), 7.87 (dd, J=7.6 Hz, J=1.6 Hz, 1H), 7.81-7.78 (m, 1H), 7.61-7.58 (m, 1H), 5.53 (q, J=6.8 Hz, 1H), 1.99 (d, J=6.8 Hz, 3H).

### Preparation 7: 3-(1-(3-cyanophenyl) ethoxy)-N5-ethyl-N2-methyl-1H-pyrrole-2,5-dicarboxamide

Following the procedure in Example 1, Preparation 2, using 3-(1-bromoethyl) benzo nitrile to give title compound as off-white solid. (0.025 g, 8%). ¹H NMR: (400 MHz, DMSO) δ 11.25 (s, 1H), 8.21 (br s, 1H), 7.99 (s, 1H), 7.82-7.74 (m, 2H), 7.58 (t, J=7.6 Hz, 1H), 7.26 (d, J=4.0 Hz, 1H), 6.35 (s, 1H), 5.46-5.44 (m, 1H), 3.17 (br s, 2H), 2.86 (d, J=4.4 Hz, 3H), 1.61 (d, J=6.4 Hz, 3H), 1.06 (t, J=6.8 Hz, 3H). LCMS¹: m/z=341 [M+H]⁺.

### Example 5—Racemic N⁵-ethyl-N²-methyl-3-(1-(o-tolyl) ethoxy)-1H-pyrrole-2,5-dicarboxamide

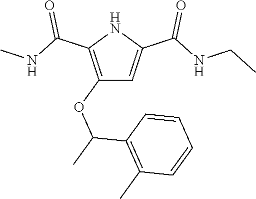 [SMILES: CCNC(=O)c1cc(OC(C)c2ccccc2C)c(C(=O)NC)[nH]1]

### Preparation 8: 1-(1-bromoethyl)-2-methylbenzene

Following the procedure in Example 1, Preparation 1, using 1-(o-tolyl) ethan-1-ol to give title compound as colourless liquid. (0.50 g, Crude).

### Preparation 9: N⁵-ethyl-N²-methyl-3-(1-(o-tolyl) ethoxy)-1H-pyrrole-2,5-dicarboxamide

Following the procedure in Example 1, Preparation 2, using 1-(1-bromoethyl)-2-methylbenzene to give title compound as white solid (0.040 g, 25%). ¹H NMR: (400 MHz, DMSO) δ 11.20 (s, 1H), 8.22 (t, J=5.2 Hz, 1H), 7.40-7.38 (m, 1H), 7.22 (d, J=4.8 Hz, 1H), 7.18-7.13 (m, 3H) 6.16 (s, 1H), 5.48 (q, J=6.4 Hz, 1H), 3.22-3.11 (m, 2H), 2.86 (d, J=4.8 Hz, 3H), 2.33 (s, 3H), 1.57 (d, J=6.4 Hz, 3H), 1.05 (t, J=7.2 Hz, 3H). LCMS¹: m/z=328 [M−H]⁻.

### Example 6—Racemic 3-(1-(2-chlorophenyl) ethoxy)-N⁵-ethyl-N²-methyl-1H-pyrrole-2,5-dicarboxamide

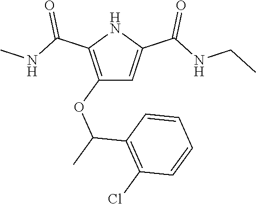 [SMILES: CCNC(=O)c1cc(OC(C)c2ccccc2Cl)c(C(=O)NC)[nH]1]

### Preparation 10: (1-bromoethyl)-2-chlorobenzene

Following the procedure in Example 1, Preparation 1, using 1-(2-chlorophenyl) ethan-1-ol to give title compound as yellow liquid (1.3 g, crude). ¹H NMR: (400 MHz, DMSO) δ 7.76 (d, 7.6 Hz, 1H), 7.49-7.35 (m, 3H), 5.66 (q, J=6.8 Hz, 1H), 2.04 (d, J=6.8 Hz, 3H)

### Preparation 11: 3-(1-(2-chlorophenyl) ethoxy)-N5-ethyl-N2-methyl-1H-pyrrole-2,5-dicarboxamide

Following the procedure in Example 1, Preparation 2, using 1-(1-bromoethyl)-2-chlorobenzene to give title compound as off-white solid (0.016 g, 15%). ¹H NMR: (400 MHz, DMSO) δ 11.35 (s, 1H), 8.28 (br s, 1H), 7.57 (d, J=6.4 Hz, 1H), 7.46 (d, J=8.0 Hz, 1H), 7.37-7.23 (m, 3H), 6.14 (s, 1H), 5.58 (q, J=6.4 Hz, 1H), 3.18-3.13 (m, 2H), 2.86 (d, J=4.4 Hz, 3H), 1.62 (d, J=6.0 Hz, 3H), 1.05 (t, J=7.2 Hz, 3H). LCMS¹: m/z=348 [M−H]⁻.

### Example 7—Racemic N⁵-ethyl-3-(1-(3-methoxyphenyl) ethoxy)-N²-methyl-1H-pyrrole-2,5-dicarboxamide

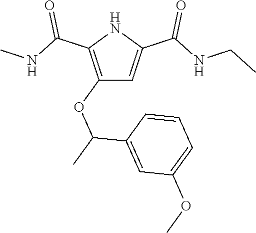 [SMILES: CCNC(=O)c1cc(OC(C)c2cccc(OC)c2)c(C(=O)NC)[nH]1]

### Preparation 40: 1-(1-bromoethyl)-3-methoxybenzene

Following the procedure in Example 1, Preparation 1, using 1-(1-bromoethyl)-3-methoxybenzene to give title compound as colourless oil (0.37 g, 37%). ¹H NMR: (400 MHz, DMSO) δ 7.32-7.26 (m, 1H), 7.09-7.02 (m, 2H), 6.88 (dd, J=7.9, 2.2 Hz, 1H), 5.45 (q, J=6.8 Hz, 1H), 3.74 (s, 3H), 1.97 (d, J=6.8 Hz, 3H).

### Preparation 12: N⁵-ethyl-3-(1-(3-methoxyphenyl) ethoxy)-N²-methyl-1H-pyrrole-2,5-dicarboxamide

Following the procedure in Example 1, Preparation 2, using 1-(1-bromoethyl)-3-methoxybenzene to give title compound as an off-white solid (0.060 g, 18%). ¹H NMR: (400 MHz, DMSO) δ 11.21 (s, 1H), 8.20 (t, J=4.8 Hz, 1H), 7.28-7.21 (m, 2H), 7.01-7.00 (m, 2H), 6.83-6.81 (m, 1H), 6.34 (d, J=1.6 Hz, 1H), 5.34 (q, J=6.4 Hz, 1H), 3.73 (s, 3H), 3.21-3.14 (m, 2H), 2.86 (d, J=4.8 Hz, 3H), 1.59 (d, J=6.4, 3H), 1.06 (t, J=7.2 Hz, 3H). LCMS¹: m/z=344 [M−H]⁻.

### Example 8—Racemic N⁵-ethyl-3-(3-methoxy-1-phenylpropoxy)-N²-methyl-1H-pyrrole-2, 5-dicarboxamide

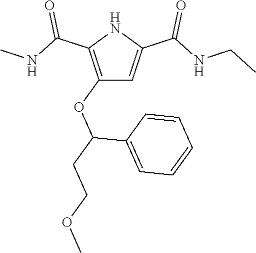 [SMILES: CCNC(=O)c1cc(OC(CCOC)c2ccccc2)c(C(=O)NC)[nH]1]

### Preparation 13: 3-methoxy-1-phenylpropan-1-one

3-chloro-1-phenylpropan-1-one (1 g, 5.95 mmol) was dissolved in methanol (17 mL). Sodium Iodide (0.89 g, 5.95 mmol) was added to the reaction mixture. The reaction was stirred at 80° C. for 16 h. The resulting suspension was filtered and concentrated under vacuum and then partitioned between water (100 mL) and ethyl acetate (100 mL). The organic layer was washed with brine solution (100 mL), dried over anhydrous sodium sulphate and concentrated under vacuum to afford crude material. The crude material was purified by flash chromatography with product eluting in 10% ethyl acetate in hexane. Fraction was combined and concentrated to give title compound as a colourless liquid (0.80 g, 82%). LCMS¹: m/z=164 [M+H]⁺.

### Preparation 14: 3-methoxy-1-phenylpropan-1-ol

Following the procedure in Example 10, Preparation 19, using 3-methoxy-1-phenylpropan-1-one to give title compound as a colourless liquid (0.36 g, 47%). ¹H NMR: (400 MHz, CDCl₃) δ 7.40-7.27 (m, 5H), 4.93 (q, J=4.0 Hz, 1H), 3.65-3.55 (m, 2H), 3.40 (s, 3H), 2.10-1.95 (m, 3H).

### Preparation 15: (1-bromo-3-methoxypropyl) benzene

Following the procedure in Example 1, Preparation 1, using 3-methoxy-1-phenylpropan-1-ol to give title compound as a colourless liquid (0.22 g, 45%). ¹H NMR: (400 MHz, CDCl₃) δ 7.39-7.28 (m, 5H), 5.29 (q, J=6.0 Hz, 1H), 3.60-3.54 (m, 1H), 3.45-3.40 (m, 1H), 3.23 (s, 3H), 2.56-2.47 (m, 1H), 2.39-2.31 (m, 1H).

### Preparation 16: N⁵-ethyl-3-(3-methoxy-1-phenylpropoxy)-N²-methyl-1H-pyrrole-2, 5-dicarboxamide

Following the procedure in Example 1, Preparation 2, using (1-bromo-3-methoxypropyl) benzene to give title compound as an off-white solid (0.055 g, 32%). ¹H NMR: (400 MHz, DMSO) δ 11.17 (s, 1H), 8.21 (t, J=5.2 Hz, 1H), 7.41 (d, J=6.8 Hz, 2H), 7.37-7.25 (m, 4H), 6.25 (s, 1H), 5.26 (q, J=4.8 Hz, 1H), 3.47-3.43 (m, 2H), 3.24 (s, 3H), 3.22-3.12 (m, 2H), 2.86 (d, J=4.8 Hz, 3H), 2.32-2.25 (m, 1H), 2.05-2.00 (m, 1H), 1.05 (t, J=7.2 Hz, 3H). LCMS¹: m/z=360 [M+H]⁺.

### Example 9—Racemic 3-(1-(4-cyanophenyl) ethoxy)-N⁵-ethyl-N²-methyl-1H-pyrrole-2, 5-dicarboxamide

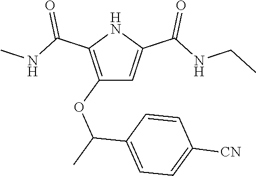 [SMILES: CCNC(=O)c1cc(OC(C)c2ccc(C#N)cc2)c(C(=O)NC)[nH]1]

### Preparation 17: 4-(1-bromoethyl) benzonitrile

4-ethylbenzonitrile (0.20 g, 1.52 mmol) was dissolved in carbon tetrachloride (4 mL) under nitrogen. NBS (0.27 g, 1.52 mmol) was portion wise added to the reaction mixture at 0° C. The reaction was stirred at 0° C. for 5 min then benzoyl peroxide (0.018 g, 0.076 mmol) was added. The reaction mixture was heated at 80° C. for 4 h. The resulting suspension was quenched with saturated sodium thiosulfate solution (30 mL) and extracted with DCM (2×30 mL). The organic layer was washed with brine solution (2×30 mL), dried over anhydrous sodium sulphate and concentrated under vacuum to afford crude material. The crude material was purified by silica flash chromatography with product eluting with 5% ethyl acetate in hexane. Fraction was combined and concentrated to give title product as an off-white liquid (0.27 g, 84%). ¹H NMR: (400 MHz, DMSO) δ 7.86 (d, J=8.4 Hz, 2H), 7.72 (d, J=8.4 Hz, 2H), 5.55 (q, J=7.2 Hz, 1H), 1.98 (d, J=7.2 Hz, 3H).

### Preparation 18: 3-(1-(4-cyanophenyl) ethoxy)-N5-ethyl-N2-methyl-1H-pyrrole-2, 5-dicarboxamide

Following the procedure in Example 1, Preparation 2, using 4-(1-bromoethyl) benzonitrile to give title compound as off-white solid (0.025 g, 22%). ¹H NMR: (400 MHz, DMSO) δ 11.32 (br s, 1H), 8.21 (s, 1H), 7.83 (d, J=8.4 Hz, 2H), 7.66 (d, J=8.0 Hz, 2H), 7.28-7.27 (m, 1H), 6.28 (s, 1H), 5.48 (q, J=7.2 Hz, 1H), 3.18-3.13 (m, 2H), 2.84 (d, J=4.4 Hz, 3H), 1.59 (d, J=6.4 Hz, 3H), 1.05 (t, J=7.2 Hz, 3H). LCMS¹: m/z=341 [M+H]⁺.

### Example 10—Racemic N⁵-ethyl-3-(2-methoxy-1-phenylethoxy)-N²-methyl-1H-pyrrole-2,5-dicarboxamide

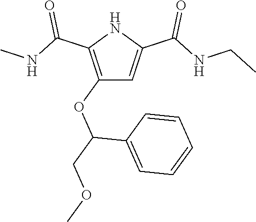 [SMILES: CCNC(=O)c1cc(OC(COC)c2ccccc2)c(C(=O)NC)[nH]1]

### Preparation 19: 2-methoxy-1-phenylethan-1-ol

2-methoxy-1-phenylethan-1-one (2 g, 13.33 mmol) was dissolved in methanol (30 mL) at room temperature. The reaction mixture was cooled at 0° C. and sodium borohydride (1.0 g, 26.66 mmol) was added portion wise under a nitrogen atmosphere. The resulting mixture was allowed to stir at room temperature for 2 h. The resulting solution was diluted with water (100 mL) and extracted with ethyl acetate (2×100 mL). The combined organics were dried over sodium sulphate, filtered and concentrated under reduced pressure to afford crude material as orange oil. The crude material was purified silica flash chromatography with product was eluting in (10:90) ethyl acetate/hexane). Fraction was combined and concentrated to give 2-methoxy-1-phenylethan-1-ol (1.3 g, 64%) as a colourless oil. ¹H NMR: (400 MHz, DMSO) δ 7.36-7.22 (m, 5H), 5.36 (d, J=4.4 Hz, 1H), 4.71-4.67 (m, 2H), 3.42-3.37 (s, 3H)

### Preparation 20: (1-bromo-2-methoxyethyl) benzene

Following the procedure in Example 1, Preparation 1, using 2-methoxy-1-phenylethan-1-ol to give title compound as yellow oil (1.3 g, 64%). ¹H NMR: (400 MHz, DMSO-d⁶) δ 7.50-7.47 (m, 2H), 7.39-7.30 (m, 3H), 5.38 (t, J=6.8 Hz, 1H), 3.91-3.83 (m, 2H), 3.37 (s, 3H).

### Preparation 21: N⁵-ethyl-3-(2-methoxy-1-phenylethoxy)-N²-methyl-1H-pyrrole-2,5-dicarboxamide

Following the procedure in Example 1, Preparation 2, using (1-bromo-2-methoxyethyl) benzene to give title compound as light grey solid (0.010 g, 3%). ¹H NMR: (400 MHz, DMSO) δ 11.27 (s, 1H), 8.20 (t, J=5.2 Hz, 1H), 7.48 (d, J=7.2 Hz, 3H), 7.40-7.31 (m, 3H), 6.14 (s, 1H), 5.26 (q, J=2.8 Hz, 1H), 3.84 (m, 2H), 3.28 (s, 3H), 3.16 (q, J=5.6 Hz, 2H), 2.86 (d, J=4.8 Hz, 3H), 1.05 (t, J=7.2 Hz, 3H). LCMS¹: m/z=346 [M+H]⁺.

### Example 11—Racemic 3-(1-(2-cyanophenyl) ethoxy)-N⁵-ethyl-N⁵-methyl-1H-pyrrole-2,5-dicarboxamide

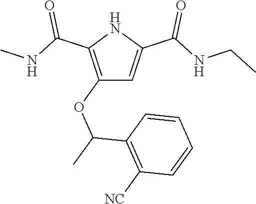 [SMILES: CCNC(=O)c1cc(OC(C)c2ccccc2C#N)c(C(=O)NC)[nH]1]

### Preparation 22: 2-(1-bromoethyl)benzonitrile

Following the procedure in Example 9, Preparation 17, using 2-ethylbenzonitrile to give title compound as colourless oil (0.75 g, 46%). ¹H NMR: (400 MHz, DMSO) δ 7.88-7.85 (m, 2H), 7.78-7.74 (m, 1H), 7.56-7.52 (m, 1H), 5.61 (q, J=6.8 Hz, 1H), 2.05 (d, J=6.8 Hz, 3H).

### Preparation 23: 3-(1-(2-cyanophenyl) ethoxy)-N5-ethyl-N2-methyl-1H-pyrrole-2,5-dicarboxamide

Following the procedure in Example 1, Preparation 2, using 2-(1-bromoethyl)benzonitrile to give title compound as light pink solid. (0.019 g, 6%). ¹H NMR: (400 MHz, DMSO) δ 11.34 (s, 1H), 8.24 (s, 1H), 7.87 (d, J=7.6 Hz, 1H), 7.74 (d, J=6.4 Hz, 2H), 7.52 (m, 1H), 7.25 (d, J=4.8 Hz, 1H), 6.31 (s, 1H), 5.58 (d, J=6.4 Hz, 1H), 3.19-3.16 (m, 2H), 2.84 (d, J=4.8 Hz, 3H), 1.69 (d, J=6.4 Hz, 3H), 1.06 (t, J=7.2 Hz, 3H). LCMS¹: m/z=341 [M+H]⁺.

### Example 12—Racemic 3-(1-(3,4-difluorophenyl) ethoxy)-N5-ethyl-N2-methyl-1H-pyrrole-2,5-dicarboxamide

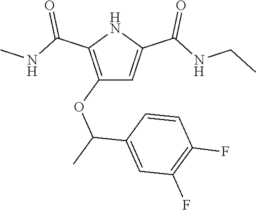 [SMILES: CCNC(=O)c1cc(OC(C)c2ccc(F)c(F)c2)c(C(=O)NC)[nH]1]

### Preparation 24: 4-(1-bromoethyl)-1,2-difluorobenzene

Following the procedure in Example 1, Preparation 1, using 1-(3,4-difluorophenyl) ethan-1-ol to give title compound as colourless oil (0.55 g, 78%). ¹H NMR: (400 MHz, DMSO) δ 7.67-7.62 (m, 1H), 7.47-7.34 (m, 2H), 5.50 (q, J=6.8 Hz, 1H), 1.97 (d, J=7.2 Hz, 3H).

### Preparation 25: 3-(1-(3,4-difluorophenyl) ethoxy)-N⁵-ethyl-N²-methyl-1H-pyrrole-2,5-dicarboxamide

Following the procedure in Example 1, Preparation 2, using 4-(1-bromoethyl)-1,2-difluorobenzene to give title compound as off-white solid (0.051 g, 15%). ¹H NMR: (400 MHz, DMSO) δ 11.25 (s, 1H), 8.21 (t, J=4.8 Hz, 1H), 7.63-7.58 (m, 1H), 7.50-7.22 (m, 3H), 6.36 (s, 1H), 5.39 (q, J=6.4 Hz, 1H), 3.37-3.20 (m, 2H), 2.85 (d, J=4.4 Hz, 3H), 1.58 (d, J=6.4 Hz, 3H), 1.06 (t, J=7.6 Hz, 3H). LCMS¹: m/z=352 [M+H]⁺.

### Example 13—Racemic 3-(1-(2,3-difluorophenyl) ethoxy)-N⁵-ethyl-N²-methyl-1H-pyrrole-2,5-dicarboxamide

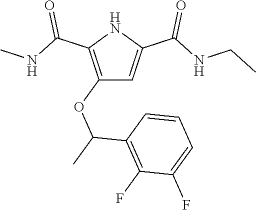 [SMILES: CCNC(=O)c1cc(OC(C)c2cccc(F)c2F)c(C(=O)NC)[nH]1]

### Preparation 26: 1-(1-bromoethyl)-2,3-difluorobenzene

Following the procedure in Example 1, Preparation 1, using 1-(2,3-difluorophenyl) ethan-1-ol to give title compound as colourless liquid. (0.30 g, Crude). ¹H NMR: (400 MHz, DMSO) δ 7.45-7.33 (m, 2H), 7.28-7.22 (m, 1H), 5.62 (q, J=6.8 Hz, 1H), 2.02 (d, J=6.8 Hz, 3H).

### Preparation 27: 3-(1-(2,3-difluorophenyl) ethoxy)-N⁵-ethyl-N²-methyl-1H-pyrrole-2,5-dicarboxamide

Following the procedure in Example 1, Preparation 2, using 1-(1-bromoethyl)-2,3-difluorobenzene to give title compound as white solid (0.02 g, 11%). ¹H NMR: (400 MHz, DMSO) δ 11.28 (s, 1H), 8.23 (t, J=4.8 Hz, 1H), 7.40-7.34 (m, 2H), 7.24-7.19 (m, 2H), 6.34 (s, 1H), 5.61 (q, J=6.4 Hz, 1H), 3.17-3.14 (m, 2H), 2.85 (d, J=4.8 Hz, 3H), 1.65 (d, J=6.4 Hz, 3H) 1.11-1.09 (t, J=7.2 Hz, 3H). LCMS¹: m/z=352 [M+H]⁺.

### Example 14—Racemic 3-(1-(2,4-difluorophenyl) ethoxy)-N⁵-ethyl-N²-methyl-1H-pyrrole-2,5-dicarboxamide

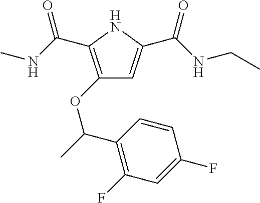 [SMILES: CCNC(=O)c1cc(OC(C)c2ccc(F)cc2F)c(C(=O)NC)[nH]1]

### Preparation 28: 1-(1-bromoethyl)-2,4-difluorobenzene

Following the procedure in Example 1, Preparation 1, using 1-(1-bromoethyl)-2,4-difluorobenzene to give title compound as yellow oil. (0.27 g, Crude). ¹H NMR: (400 MHz, DMSO) δ 7.73-6.79 (m, 1H), 7.31-7.26 (m, 1H), 7.15-7.10 (m, 1H), 5.59 (d, J=6.8 Hz, 1H), 1.99 (d, J=5.6 HZ, 3H).

### Preparation 29: 3-(1-(2,4-difluorophenyl) ethoxy)-N⁵-ethyl-N²-methyl-1H-pyrrole-2,5-dicarboxamide

Following the procedure in Example 1, Preparation 2, using 1-(1-bromoethyl)-2,4-difluorobenzene to give title compound as off-white solid (0.072 g, 50%). ¹H NMR: (400 MHz, DMSO) δ 11.26 (s, 1H), 8.23 (t, J=5.2 Hz, 1H), 7.60 (m, 1H), 7.30-7.21 (m, 2H), 7.13-7.10 (m, 1H), 6.34 (s, 1H), 5.55-5.54 (m, 1H), 3.20-3.16 (m, 2H), 2.84 (d, J=4.8 Hz, 3H), 1.62 (d, J=6.4 Hz, 3H), 1.06 (t, J=7.2 Hz, 3H). LCMS¹: m/z=352 [M+H]⁺.

### Example 15—Racemic 3-(1-(1,3-dihydroisobenzofuran-5-yl) ethoxy)-N⁵-ethyl-N²-methyl-1H-pyrrole-2,5-dicarboxamide

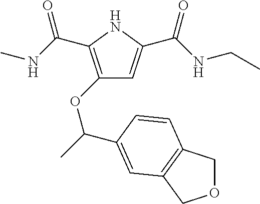 [SMILES: CCNC(=O)c1cc(OC(C)c2ccc3c(c2)COC3)c(C(=O)NC)[nH]1]

### Preparation 30: 1-(1,3-dihydroisobenzofuran-5-yl) ethan-1-ol

3-(prop-2-yn-1-yloxy) prop-1-yne (1.0 g, 10.63 mmol) and but-3-yn-2-ol (3.72 g, 53.19 mmol) was added ruthenium trichloride (0.022 g, 0.10 mmol) at room temperature under nitrogen atmosphere. The suspension was allowed to stir at 120° C. for 16 h. The reaction mixture was slowly added to water (200 mL) and extracted with EtOAc (200 mL). The organic layer was washed with brine (200 mL) and dried over anhydrous Na₂SO₄ and concentrated under vacuum reduced pressure to afford crude material. The crude material was purified by silica flash chromatography eluting with (20:80) ethyl acetate/Hexane). Fraction was combined and concentrated to give 1-(1,3-dihydroisobenzofuran-5-yl) ethan-1-ol (0.90 g, 52%) as a yellow solid. ¹H NMR: (400 MHz, DMSO) δ 7.27-7.21 (m, 3H), 5.16 (d, J=4.4 Hz, 1H), 4.97 (s, 4H), 4.76-4.70 (m, 1H), 1.31 (d, J=6.4 Hz, 3H).

### Preparation 31: 5-(1-bromoethyl)-1,3-dihydroisobenzofuran

Following the procedure in Example 1, Preparation 1, using 1-(1,3-dihydroisobenzofuran-5-yl) ethan-1-ol to give title compound as colourless liquid (0.80 g, Crude). ¹H NMR: (400 MHz, DMSO) δ 7.78-7.27 (m, 3H), 5.55 (q, J=6.8 Hz, 1H), 5.05-4.98 (m, 4H), 1.99 (d, J=6.8 Hz, 3H)

### Preparation 32: 3-(1-(1,3-dihydroisobenzofuran-5-yl) ethoxy)-N⁵-ethyl-N²-methyl-1H-pyrrole-2,5-dicarboxamide

Following the procedure in Example 1, Preparation 2, using 5-(1-bromoethyl)-1,3-dihydroisobenzofuran to give title compound as white solid (0.020 g, 15%). ¹H NMR: (400 MHz, DMSO) δ 11.10 (s, 1H), 8.21 (t, J=4.8 Hz, 1H), 7.38-7.34 (m, 2H), 7.28 (d, J=7.8 Hz, 1H), 7.21-7.20 (m, 1H), 6.32 (s, 1H), 5.38 (q, J=6.4 Hz, 1H), 4.92 (s, 4H), 3.21-3.14 (m, 2H), 2.87 (d, J=4.8 Hz, 3H), 1.59 (d, J=6.4 Hz, 3H) 1.04 (t, J=7.2 Hz, 3H). LCMS¹: m/z=356 [M−H]⁻.

### Example 16—Racemic N⁵-ethyl-N²-methyl-3-(1-phenylbutoxy)-1H-pyrrole-2,5-dicarboxamide

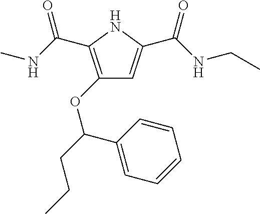 [SMILES: CCCC(Oc1cc(C(=O)NCC)[nH]c1C(=O)NC)c1ccccc1]

### Preparation 33: (1-bromobutyl) benzene

Following the procedure in Example 1, Preparation 1, using 1-phenylbutan-1-ol to give title compound as colourless oil. (0.60 g, 84%). ¹H NMR: (400 MHz, DMSO) δ 7.48-7.46 (m, 4H), 7.32-7.28 (m, 1H), 5.27 (t, J=7.6 Hz, 1H), 2.24-2.07 (m, 2H), 1.42-1.23 (m, 2H), 0.89 (t, J=7.6 Hz, 3H).

### Preparation 34: N⁵-ethyl-N²-methyl-3-(1-phenylbutoxy)-1H-pyrrole-2,5-dicarboxamide

Following the procedure in Example 1, Preparation 2, using (1-bromobutyl) benzene to give title compound as off-white solid (0.014 g, 4%). 1H NMR: (400 MHz, DMSO) δ 11.19 (s, 1H), 8.20 (s, 1H), 7.42 (d, J=7.2 Hz, 2H), 7.34 (apparent t, J=7.2, 2H), 7.26 (d, J=7.2 Hz, 1H), 7.20 (d, J=4.8 Hz, 1H), 6.31 (s, 1H), 5.18 (q, J=6.8 Hz, 1H), 3.16 (br s, 2H), 2.86 (d, J=4.8 Hz, 3H), 2.06-2.03 (m, 1H) 1.79-1.73 (m, 1H), 1.43-1.16 (m, 2H), 1.05 (t, J=7.2 Hz, 3H), 0.91 (t, J=7.2 Hz, 3H). LCMS¹: m/z=366 [M+Na]⁺.

### Example 17—Racemic N⁵-ethyl-N²-methyl-3-(1-(m-tolyl) ethoxy)-1H-pyrrole-2,5-dicarboxamide

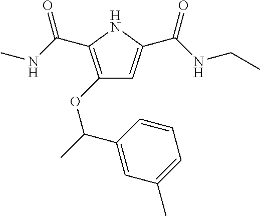 [SMILES: CCNC(=O)c1cc(OC(C)c2cccc(C)c2)c(C(=O)NC)[nH]1]

### Preparation 35: 1-(1-bromoethyl)-3-methylbenzene

Following the procedure in Example 1, Preparation 1, using 1-(m-tolyl) ethan-1-ol to give title compound colourless oil (0.30 g, 41%). ¹H NMR: (400 MHz, DMSO) δ 7.31-7.27 (m, 4H), 5.51-5.42 (m, 1H), 2.31 (s, 3H), 1.99 (d, J=6.8 Hz, 3H).

### Preparation 36: N⁵-ethyl-N²-methyl-3-(1-(m-tolyl) ethoxy)-1H-pyrrole-2,5-dicarboxamide

Following the procedure in Example 1, Preparation 2, using 1-(1-bromoethyl)-3-methylbenzene to give title compound as an off-white solid (0.015 g, 9%). ¹H NMR: (400 MHz, DMSO) δ 11.21 (s, 1H), 8.20 (t, J=5.2 Hz, 1H), 7.25-7.20 (m, 4H), 7.07 (d, J=6.0 Hz, 1H), 6.31 (s, 1H), 5.31 (q, J=6.4 Hz, 1H), 3.19-3.15 (m, 2H), 2.86 (d, J=4.8 Hz, 3H), 2.29 (s, 3H), 1.58 (d, J=6.4 Hz, 3H), 1.06 (t, J=7.2 Hz, 3H). LCMS¹: m/z=328 [M−H]⁻.

### Example 18—3-(1-(4-chlorophenyl) ethoxy)-N⁵-ethyl-N²-methyl-1H-pyrrole-2,5-dicarboxamide, Enantiomer A

### Example 19—3-(1-(4-chlorophenyl) ethoxy)-N⁵-ethyl-N²-methyl-1H-pyrrole-2,5-dicarboxamide, Enantiomer B

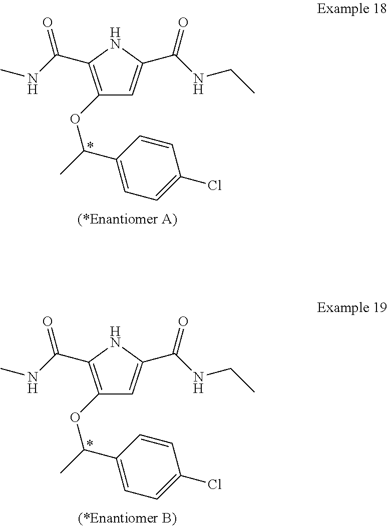

Example 122 was separated by Chiral Prep-HPLC¹⁵ to afford Enantiomer A (0.040 g, 12%) as an off-white solid and Enantiomer B (0.04 g, 12%) as an off-white solid.

Example 18 (Enantiomer A): ¹H NMR: (400 MHz, DMSO) δ 11.23 (s, 1H), 8.20 (t, J=4.8 Hz, 1H), 7.49 (d, J=8.4 Hz, 2H), 7.41 (d, J=8.4 Hz, 2H), 7.21-7.20 (m, 1H), 6.32 (s, 1H), 5.39 (q, J=6.4 Hz, 1H), 3.17 (t, J=4.8 Hz, 2H), 2.85 (d, J=4.4 Hz, 3H), 1.58 (d, J=6.4 Hz, 3H) 1.06 (t, J=7.2 Hz, 3H). LCMS¹: m/z=348 [M−H]⁻. Chiral HPLC⁵ t_{R}: 3.51 min, ee %: 100%. The absolute configuration has not yet been assigned unambiguously but is believed to be the S enantiomer.

Example 19 (Enantiomer B): LCMS¹: m/z=348 [M−H]⁻. Chiral HPLC⁵ t_{R}: 3.91 min, ee %: 100%. The absolute configuration has not yet been assigned unambiguously but is believed to be the R enantiomer.

### Example 20—N⁵-ethyl-3-(1-(2-fluorophenyl) ethoxy)-N²-methyl-1H-pyrrole-2,5-dicarboxamide, Enantiomer A

### Example 21—N⁵-ethyl-3-(1-(2-fluorophenyl) ethoxy)-N²-methyl-1H-pyrrole-2,5-dicarboxamide, Enantiomer B

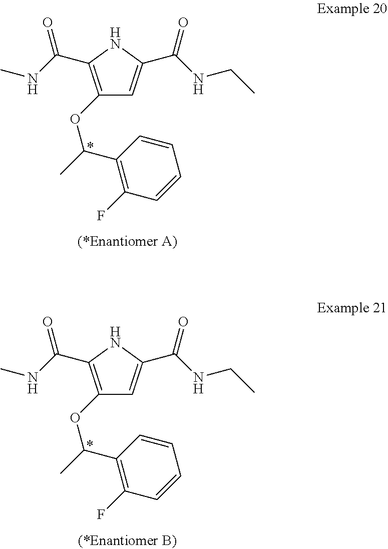

Example 124 was separated by Chiral Prep-HPLC¹⁴ to afford Enantiomer A (0.035 g, 11%) as an off-white solid and Enantiomer B (0.04 g, 12%) as an off-white solid.

Example 20 (Enantiomer A): ¹H NMR: (400 MHz, DMSO) δ 11.25 (s, 1H), 8.22 (t, J=4.8 Hz, 1H), 7.53 (apparent t, J=7.6 Hz, 1H), 7.36-7.32 (m, 1H), 7.24-7.18 (m, 3H), 6.32 (s, 1H), 5.57 (q, J=6.0 Hz, 1H), 3.20-3.14 (m, 2H), 2.85 (d, J=4.8 Hz, 3H), 1.64 (d, J=6.4 Hz, 3H), 1.06 (t, J=7.2 Hz, 3H). LCMS¹: m/z=334 [M+H]⁺. Chiral HPLC⁴ t_{R}: 2.27 min, ee %: 100%. The absolute configuration has not yet been assigned unambiguously but is believed to be the S enantiomer.

Example 21 (Enantiomer B): ¹H NMR: (400 MHz, DMSO) δ 11.25 (s, 1H), 8.22 (t, J=4.8 Hz, 1H), 7.53 (apparent t, J=7.6 Hz, 1H), 7.36-7.32 (m, 1H), 7.24-7.18 (m, 3H), 6.32 (s, 1H), 5.57 (q, J=6.0 Hz, 1H), 3.20-3.14 (m, 2H), 2.85 (d, J=4.8 Hz, 3H), 1.64 (d, J=6.4 Hz, 3H), 1.06 (t, J=7.2 Hz, 3H). LCMS¹: m/z=334 [M+H]⁺. Chiral HPLC⁴ t_{R}: 2.60 min, ee %: 97.7%. The absolute configuration has not yet been assigned unambiguously but is believed to be the R enantiomer.

### Example 22—Racemic 3-(1-(1,3-dihydroisobenzofuran-4-yl) ethoxy)-N⁵-ethyl-N²-methyl-1H-pyrrole-2,5-dicarboxamide

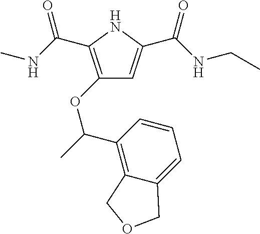 [SMILES: CCNC(=O)c1cc(OC(C)c2cccc3c2COC3)c(C(=O)NC)[nH]1]

### Preparation 37: 1,3-dihydroisobenzofuran-4-carbaldehyde

4-bromo-1,3-dihydroisobenzofuran (2.0 g, 10.15 mmol) was dissolved in THE (2 mL) under nitrogen at 0° C. Isopropyl magnesium chloride (10.05 mL, 20.30 mmol) was dropwise added to the reaction mixture at 0° C. and stirred at 0° C. for 10 min. n-Butyllithium (6.8 mL, 15.22 mmol) was dropwise added to the reaction mixture at 0° C. and stirred at 0° C. for 1 h. DMF (0.83 g, 15.22 mmol) was dropwise added to the reaction mixture at 0° C. and reaction warmed to room temperature and stirred for 16 h. The resulting suspension was diluted with water (200 mL) and extracted with ethyl acetate (2×200 mL). The organic layer was washed with brine solution (2×200 mL) and concentrated under vacuum to afford crude material. The crude material was purified by flash chromatography in normal phase eluting with 10% ethyl acetate in hexane. Fraction was combined and concentrated to give title product as a colourless liquid (0.15 g, 23%). LCMS¹: m/z=149 [M+H]⁺.

### Preparation 38: 1-(1,3-dihydroisobenzofuran-4-yl) ethan-1-ol

1,3-dihydroisobenzofuran-4-carbaldehyde (0.15 g, 1.01 mmol) was dissolved in THE (2 mL) under nitrogen. 2 M Methyl magnesium bromide in THE (0.7 mL, 1.51 mmol) was dropwise added to the reaction mixture at 0° C. The reaction was stirred at room temperature for 1 h. The resulting suspension was diluted with water (25 mL) and extracted with ethyl acetate (2×25 mL). The organic layer was washed with brine solution (2×25 mL) and concentrated to give colourless liquid (0.15 g, 45%). ¹H NMR: (400 MHz, DMSO) δ 7.26-7.22 (m, 2H), 7.16 (d, J=6.8 Hz, 1H), 5.21 (d, J=3.6 Hz, 2H), 5.07 (d, J=2.0 Hz, 2H), 4.71 (q, J=3.6 Hz, 1H), 1.27 (d, J=6.8 Hz, 3H).

### Preparation 39: 4-(1-bromoethyl)-1,3-dihydroisobenzofuran

Following the procedure in Example 1, Preparation 1, using 1-(1,3-dihydroisobenzofuran-4-yl) ethan-1-ol to give tile compound as colourless liquid (0.17 g, Crude).

### Preparation 40: 3-(1-(1,3-dihydroisobenzofuran-4-yl) ethoxy)-N5-ethyl-N2-methyl-1H-pyrrole-2,5-dicarboxamide

Following the procedure in Example 1, Preparation 2, using 4-(1-bromoethyl)-1,3-dihydroisobenzofuran to give title compound as off-white solid (0.03 g, 8%). %). ¹H NMR: (400 MHz, DMSO) δ 11.25 (s, 1H), 8.21 (br s, 1H), 7.34-7.29 (m, 2H), 7.22-7.17 (m, 2H), 6.22 (s, 1H), 5.36 (d, J=6.8 Hz, 1H), 4.97-4.88 (m, 4H), 3.19-3.14 (m, 2H), 2.86 (d, J=4.4 Hz, 3H), 1.57 (d, J=6.4 Hz, 3H), 1.05 (t, J=7.2 Hz, 3H). LCMS¹: m/z=358 [M+H]⁺.

### Example 23—(S)—N-methyl-5-(5-methyl-1H-imidazol-2-yl)-3-(1-phenylethoxy)-1H-pyrrole-2-carboxamide

### Example 24—(R)—N-methyl-5-(5-methyl-1H-imidazol-2-yl)-3-(1-phenylethoxy)-1H-pyrrole-2-carboxamide

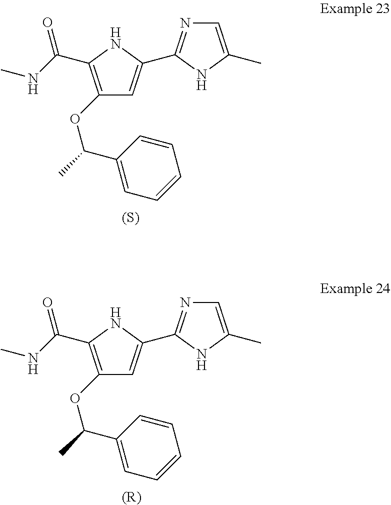

### Preparation 41: Ethyl (S)-5-(methylcarbamoyl)-4-(1-phenylethoxy)-1H-pyrrole-2-carboxylate

To a stirred mixture of ethyl 4-hydroxy-5-(methylcarbamoyl)-1H-pyrrole-2-carboxylate (6.00 g, 28.2 mmol) and (R)-1-phenylethan-1-ol (6.91 g, 56.5 mmol) in THE (80.0 mL) were added PPh₃ (11.1 g, 42.4 mmol) at room temperature under nitrogen atmosphere. The resulting mixture was stirred for 30 min and followed by the addition of DIAD (8.58 g, 42.4 mmol) dropwise. The resulting mixture was stirred at room temperature for additional 2 h. The resulting mixture was extracted with EtOAc (3×50.0 mL). The combined organic phases were washed with brine and dried over anhydrous Na₂SO₄. After filtration, the filtrate was concentrated under reduced pressure. The residue was purified by silica gel column chromatography, eluted with PE/EA (1:1) to afford the title compound as a yellow oil (5.50 g, 61.4%). LCMS: m/z=317 [M+H]⁺

### Preparation 42: (S)-5-(hydroxymethyl)-N-methyl-3-(1-phenylethoxy)-1H-pyrrole-2-carboxamide

To a stirred mixture of ethyl (S)-5-(methylcarbamoyl)-4-(1-phenylethoxy)-1H-pyrrole-2-carboxylate (2.50 g, 7.90 mmol) in THE (25.0 mL) was added 1.5 M DIBAL-H in DCM (22.5 mL, 31.6 mmol) at −40° C. under nitrogen atmosphere. The resulting mixture was stirred at −40° C. for 3 h and quenched by the addition of 1 M aq. HCl. The resulting mixture was extracted with EtOAc (3×40.0 mL). The combined organic phases were washed with brine and dried over anhydrous Na₂SO₄. After filtration, the filtrate was concentrated under reduced pressure. The residue was purified by silica gel column chromatography, eluted with PE/EA (2:3) to afford the title compound as a red oil (1.50 g, 69.1%). LCMS: m/z=275 [M+H]⁺

### Preparation 43: (S)-5-formyl-N-methyl-3-(1-phenylethoxy)-1H-pyrrole-2-carboxamide

To a stirred mixture of (S)-5-(hydroxymethyl)-N-methyl-3-(1-phenylethoxy)-1H-pyrrole-2-carboxamide (1.50 g, 5.46 mmol) in DCM (20.0 mL) was added Dess-Martin reagent (2.78 g, 6.56 mmol). The resulting mixture was stirred at room temperature for 2 h. The resulting mixture was extracted with CH₂Cl₂ (3×20.0 mL). The combined organic phases were washed with brine (20.0 mL) and dried over anhydrous Na₂SO₄. After filtration, the filtrate was concentrated under reduced pressure. The residue was purified by silica gel column chromatography, eluted with PE/EA (1:1) to afford the title compound as a yellow solid (900 mg, 60.4%). LCMS: m/z=273 [M+H]⁺.

### Preparation 44: (S)- and (R)—N-methyl-5-(5-methyl-1H-imidazol-2-yl)-3-(1-phenylethoxy)-1H-pyrrole-2-carboxamide

To a stirred solution of (S)-5-formyl-N-methyl-3-(1-phenylethoxy)-1H-pyrrole-2-carboxamide (900 mg, 3.30 mmol) and 2-oxopropanal (600 mg, 8.33 mmol) in EtOH (9.00 mL) was added NH₃·H₂O (4.32 mL, 30% wt). The resulting mixture was stirred at 100° C. overnight. The resulting mixture was concentrated under reduced pressure. The residue was purified by Prep-HPLC^{H} to afford crude product (650 mg). Chiral HPLC analysis indicated a small amount of the (R) isomer was present due to epimerisation during the synthesis. The mixture was therefore separated by Chiral Prep-HPLC^{N} to afford the (S) enantiomer as a yellow solid (158 mg, 14.7%) and the (R) enantiomer as a yellow solid (20.0 mg, 1.87%)

Example 23 ¹H NMR (400 MHz, DMSO-d₆) δ 11.82 (br, 1H), 10.92 (s, 1H), 7.46-7.44 (m, 2H), 7.43-7.37 (m, 2H), 7.33-7.28 (m, 1H), 7.05 (s, 1H), 6.71 (s, 1H), 6.16 (s, 1H), 5.40-5.37 (m, 1H), 2.87 (m, 3H), 2.11 (s, 3H), 1.62 (d, J=6.4 Hz, 3H). LCMS^{J}: m/z=325 [M+H]⁺. Chiral HPLC^{G} t_{R}: 0.87 min, ee %: 100%.

Example 24 ¹H NMR (400 MHz, DMSO-d₆) δ 13.73 (br, 1H), 11.71 (s, 1H), 7.47-7.45 (m, 2H), 7.39-7.37 (m, 2H), 7.28-7.26 (m, 3H), 6.57 (s, 1H), 5.36-5.31 (m, 1H), 2.90-2.89 (m, 3H), 2.26 (s, 3H), 1.67 (d, J=6.4 Hz, 3H). LCMS^{J}: m/z 325 [M+H]⁺. Chiral HPLC^{G}t_{R}: 1.09 min, ee %: 89.65%.

### Example 25—N⁵-ethyl-N²-methyl-3-(1-(p-tolyl) ethoxy)-1H-pyrrole-2,5-dicarboxamide, Enantiomer A

### Example 26—N⁵-ethyl-N²-methyl-3-(1-(p-tolyl) ethoxy)-1H-pyrrole-2,5-dicarboxamide, Enantiomer B

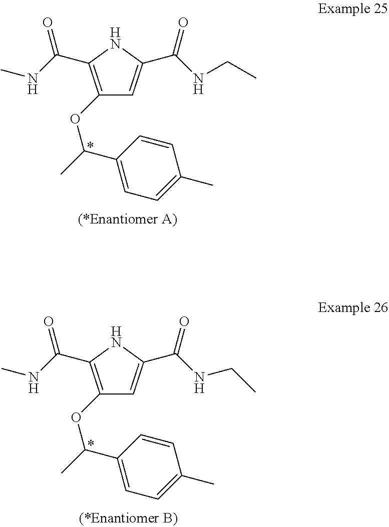

### Preparation 45: Ethyl 5-(methylcarbamoyl)-4-(1-(p-tolyl) ethoxy)-1H-pyrrole-2-carboxylate

Ethyl 4-hydroxy-5-(methylcarbamoyl)-1H-pyrrole-2-carboxylate (1.3 g, 6.13 mmol) and 1-(p-tolyl) ethan-1-ol (1.25 g, 9.19 mmol) were dissolved in THE (13 mL) at room temperature under argon. Triphenylphosphine (2.40 g, 9.19 mmol) was added to the reaction mixture and allowed to stir at room temperature for 30 min. The resulting solution was cooled at 0° C. and dropwise added Diethyl azodicarboxylate (1.85 g, 9.19 mmol). The resulting suspension was allowed to stir at room temperature for 16 h. The reaction mixture was slowly added to water (200 mL) and extracted with ethyl acetate (2×200 mL). The organic layer was washed with brine (2×150 mL) and dried over anhydrous sodium sulphate and concentrated under vacuum to afford crude material. The crude material was purified by normal phase chromatography column chromatography, eluting with (30:70) ethyl acetate/hexane. Solvent reduction gave titled product as a yellow gummy solid (1.2 g, 59%). LCMS¹: m/z=329 [M−H]⁻

### Preparation 46: 5-(methylcarbamoyl)-4-(1-(p-tolyl) ethoxy)-1H-pyrrole-2-carboxylic acid

Ethyl 5-(methylcarbamoyl)-4-(1-(p-tolyl) ethoxy)-1H-pyrrole-2-carboxylate (1.2 g, 3.63 mmol) was dissolved in methanol (24 mL) and water (12 mL) at room temperature. Lithium hydroxide (0.74 g, 18.18 mmol) was added to the reaction mixture at room temperature and allowed to stir at 60° C. for 5 h. The resulting solution was cooled to room temperature and concentrated under vacuum distillation. The resulting residue was diluted with water (100 mL) and extracted with ethyl acetate (2×100 mL). The aqueous layer was acidified using 1 N HCL solution till pH became 2. The acidic phase was extracted with ethyl acetate (2×100 mL). Fractions were combined and concentrated under vacuum to afford titled product as a pink solid. (0.75 g, 68%). LCMS¹: m/z=301 [M−H]⁻.

### Preparation 47: N⁵-ethyl-N²-methyl-3-(1-(p-tolyl) ethoxy)-1H-pyrrole-2,5-dicarboxamide

5-(methylcarbamoyl)-4-(1-(p-tolyl) ethoxy)-1H-pyrrole-2-carboxylic acid (0.75 g, 2.48 mmol) was dissolved in DMF (7.5 mL) under nitrogen at room temperature. 1,1′-Carbonyldiimidazole (0.80 g, 4.98 mmol) was added to the reaction mixture and allowed to stir at room temperature for 30 min. Ethylamine (0.22 g, 4.98 mmol) was added to the reaction mixture at room temperature. The resulting mixture was stirred at room temperature for 2 h. The reaction mixture was quenched with water (25 mL) and extracted with ethyl acetate (2×25 mL). The organic layer was washed with brine solution (2×25 mL) and concentrated under vacuum to afford crude material. The crude material was purified flash chromatography in reverse phase eluting with (60:40) acetonitrile/water. Fraction was combined and lyophilized to give white solid as mixture of enantiomers (0.40 g, 68%). The mixture of enantiomers was separated by Chiral Prep-HPLC¹⁶ to afford Enantiomer A (0.09 g, 11%) as an off-white solid and Enantiomer B (0.08 g, 9.0%) as an off-white solid.

Example 25 (Enantiomer A): ¹H NMR: (400 MHz, DMSO) δ 11.19 (br s, 1H), 8.20 (t, J=5.2 Hz, 1H), 7.31 (d, J=8.0 Hz, 2H), 7.22-7.17 (m, 1H), 7.14 (d, J=8.0 Hz, 2H), 6.31 (s, 1H), 5.32 (q, J=6.4 Hz, 1H), 3.19-3.15 (m, 2H), 2.86 (d, J=4.8 Hz, 3H), 2.26 (s, 3H), 1.58 (d, J=6.4 Hz, 3H), 1.05 (t, J=7.2 Hz, 3H). LCMS¹: m/z=328 [M−H]⁻. Chiral HPLC³ t_{R}: 6.32 min, ee %: 98.22%. The absolute configuration has not yet been assigned unambiguously but is believed to be the S enantiomer.

Example 26 (Enantiomer B): ¹H NMR: (400 MHz, DMSO) δ 11.07 (br s, 1H), 8.19 (t, J=5.2 Hz, 1H), 7.31 (d, J=8.0 Hz, 2H), 7.20-7.19 (m, 1H), 7.14 (d, J=8.0 Hz, 2H), 6.31 (s, 1H), 5.32 (q, J=6.4 Hz, 1H), 3.19-3.15 (m, 2H), 2.85 (d, J=4.8 Hz, 3H), 2.26 (s, 3H), 1.58 (d, J=6.4 Hz, 3H), 1.06 (t, J=7.2 Hz, 3H). LCMS¹: m/z=328 [M−H]⁻. Chiral HPLC³ t_{R}: 6.60 min, ee %: 99.30%. The absolute configuration has not yet been assigned unambiguously but is believed to be the R enantiomer.

### Example 27—3-(1-(4-chlorophenyl) ethoxy)-N⁵-cyclobutyl-N²-methyl-1H-pyrrole-2,5-dicarboxamide, Enantiomer A

### Example 28—3-(1-(4-chlorophenyl) ethoxy)-N⁵-cyclobutyl-N²-methyl-1H-pyrrole-2,5-dicarboxamide, Enantiomer B

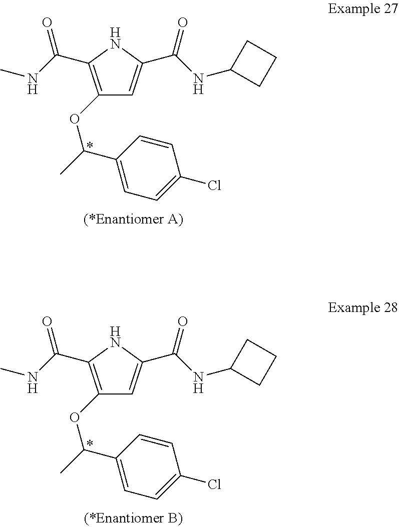 [SMILES: CNC(=O)c1[nH]c(C(=O)NC2CCC2)cc1OC(C)c1ccc(Cl)cc1 ; CNC(=O)c1[nH]c(C(=O)NC2CCC2)cc1OC(C)c1ccc(Cl)cc1]

### Preparation 48: Ethyl 4-(1-(4-chlorophenyl) ethoxy)-5-(methylcarbamoyl)-1H-pyrrole-2-carboxylate

Ethyl 4-hydroxy-5-(methylcarbamoyl)-1H-pyrrole-2-carboxylate (2.5 g, 11.79 mmol) and 1-(4-chlorophenyl) ethan-1-ol (2.77 g, 17.68 mmol) were dissolved in THE (50 mL) at room temperature under argon. Triphenylphosphine (4.63 g, 17.68 mmol) was added to the reaction mixture and allowed to stir at room temperature for 30 min. The resulting solution was cooled at 0° C. and diisopropyl azodicarboxylate (3.57 g, 17.68 mmol) was added dropwise. The resulting suspension was allowed to stir at room temperature for 4 h. The reaction mixture was slowly added to water (200 mL) and extracted with ethyl acetate (2×200 mL) and dried over anhydrous sodium sulphate and concentrated under vacuum to afford crude material. The crude material was purified by normal phase chromatography column chromatography, eluting with (35:65) ethyl acetate/hexane. Solvent reduction gave title compound as yellow gummy solid (1.6 g, 38.8%). LCMS¹: m/z=351 [M+H]⁺.

### Preparation 49: 4-(1-(4-chloro phenyl) ethoxy)-5-(methylcarbamoyl)-1H-pyrrole-2-carboxylic acid

Following the procedure in Example 25/Example 26, Preparation 46, using ethyl 4-(1-(4-chlorophenyl) ethoxy)-5-(methylcarbamoyl)-1H-pyrrole-2-carboxylate to give title compound as off-white solid. (0.75 g, 51%). ¹H NMR: (400 MHz, DMSO) δ 12.63 (br s, 1H), 11.30 (s, 1H), 7.49 (d, J=8.4 Hz, 2H), 7.41 (d, J=8.4 Hz, 2H), 7.34-7.31 (m, 1H), 6.35 (s, 1H), 5.43 (q, J=6.4 Hz, 1H), 2.83 (d, J=4.4 Hz, 3H), 1.57 (d, J=6.4 Hz, 3H). LCMS¹: m/z=323 [M+H]⁺.

### Preparation 50: 3-(1-(4-chlorophenyl) ethoxy)-N⁵-cyclobutyl-NV-methyl-1H-pyrrole-2,5-dicarboxamide

Following the procedure in Example 25/Example 26, Preparation 47 using 4-(1-(4-chloro phenyl) ethoxy)-5-(methylcarbamoyl)-1H-pyrrole-2-carboxylic acid and cyclobutylamine to give a mixture of enantiomers as white solid (0.140 g, 60%). The mixture of enantiomers was separated by Chiral Prep HPLC³ to afford Enantiomer A (0.043 g, 18%) as an off-white solid and Enantiomer B (0.045 g, 19%) as an off-white solid.

Example 27 (Enantiomer A): ¹H NMR: (400 MHz, DMSO) δ 11.29 (s, 1H), 8.43 (d, J=7.2 Hz, 1H), 7.47 (d, J=8.4 Hz, 2H), 7.41 (d, J=8.4 Hz, 2H), 7.22-7.19 (m, 1H), 6.31 (s, 1H), 5.39 (q, J=6.4 Hz, 1H), 4.26 (q, J=8.0 Hz, 1H), 2.85 (d, J=4.8 Hz, 3H), 2.19-2.17 (m, 2H), 1.99-1.89 (m, 2H), 1.68-1.59 (m, 2H), 1.90 (d, J=9.2 Hz, 3H). LCMS¹: m/z=376 [M+H]⁺. Chiral HPLC³ t_{R}: 2.58 min, ee %: 100%. The absolute configuration has not yet been assigned unambiguously but is believed to be the S enantiomer.

Example 28 (Enantiomer B): ¹H NMR: (400 MHz, DMSO) δ 11.29 (s, 1H), 8.42 (d, J=7.2 Hz, 1H), 7.47 (d, J=8.4 Hz, 2H), 7.41 (d, J=8.4 Hz, 2H), 7.21-7.20 (m, 1H), 6.32 (s, 1H), 5.39 (q, J=6.4 Hz, 1H), 4.26 (q, J=8.0 Hz, 1H), 2.85 (d, J=4.8 Hz, 3H), 2.19-2.17 (m, 2H), 1.99-1.89 (m, 2H), 1.68-1.59 (m, 2H), 1.90 (d, J=9.2 Hz, 3H). LCMS¹: m/z=376 [M+H]⁺. Chiral HPLC³ t_{R}: 3.04 min, ee %: 99.53%. The absolute configuration has not yet been assigned unambiguously but is believed to be the R enantiomer.

### Example 29—3-(1-(4-chlorophenyl) ethoxy)-N⁵-(4-hydroxycyclohexyl)-N²-methyl-1H-pyrrole-2,5-dicarboxamide, Enantiomer A

### Example 30—3-(1-(4-chlorophenyl) ethoxy)-N⁵-(4-hydroxycyclohexyl)-N²-methyl-1H-pyrrole-2,5-dicarboxamide, Enantiomer B

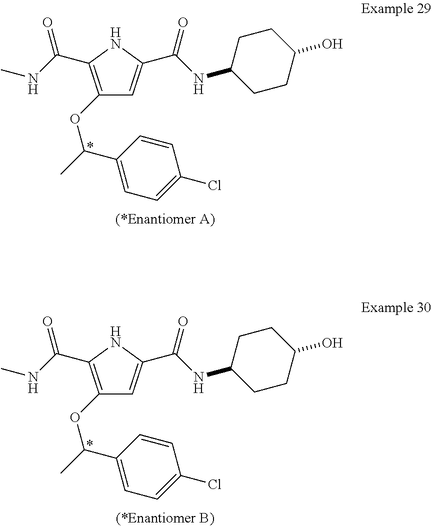

Following the procedure in Example 25/Example 26, Preparation 47 using 4-(1-(4-chloro phenyl) ethoxy)-5-(methylcarbamoyl)-1H-pyrrole-2-carboxylic acid and trans-4-aminocyclohexan-1-ol give mixture of enantiomers as white solid (0.130 g, 50%). The mixture of enantiomers was separated by Chiral Prep HPLC⁵ to afford Enantiomer A (0.048 g, 18%) as an off-white solid and Enantiomer B (0.046 g, 18%) as an off-white solid.

Example 29 (Enantiomer A): ¹H NMR: (400 MHz, DMSO-d⁶) δ 11.33 (s, 1H), 8.02 (d, J=7.2 Hz, 1H), 7.46 (d, J=8.4 Hz, 2H), 7.41 (d, J=8.4 Hz, 2H), 7.20-7.18 (m, 1H), 6.32 (s, 1H), 5.41 (q, J=6.4 Hz, 1H), 4.55 (d, J=4.4 Hz, 1H), 3.56 (br s, 1H), 3.35 (d, J=4.0 Hz, 1H), 2.85 (d, J=4.8 Hz, 3H), 1.79 (br s, 4H), 1.57 (d, J=6.4 Hz, 3H), 1.23-1.17 (m, 4H). LCMS¹: m/z=420 [M+H]⁺. Chiral HPLC¹⁰ t_{R}: 12.74 min, ee %: 96.55%. The absolute configuration has not yet been assigned unambiguously but is believed to be the S enantiomer.

Example 30 (Enantiomer B): ¹H NMR: (400 MHz, DMSO) δ 11.33 (s, 1H), 8.02 (d, J=7.2 Hz, 1H), 7.46 (d, J=8.4 Hz, 2H), 7.41 (d, J=8.4 Hz, 2H), 7.20-7.18 (m, 1H), 6.32 (s, 1H), 5.41 (q, J=6.4 Hz, 1H), 4.55 (d, J=4.4 Hz, 1H), 3.56 (br s, 1H), 3.35 (d, J=4.0 Hz, 1H), 2.85 (d, J=4.8 Hz, 3H), 1.79 (br s, 4H), 1.57 (d, J=6.4 Hz, 3H), 1.23-1.17 (m, 4H). LCMS¹: m/z=420 [M+H]⁺. Chiral HPLC¹⁰ t_{R}: 12.81 min, ee %: 99.66%. The absolute configuration has not yet been assigned unambiguously but is believed to be the R enantiomer.

### Example 31—3-(1-(4-chlorophenyl) ethoxy)-N²-methyl-N⁵-(oxetan-3-yl)-1H-pyrrole-2,5-dicarboxamide, Enantiomer A

### Example 32—3-(1-(4-chlorophenyl) ethoxy)-N²-methyl-N⁵-(oxetan-3-yl)-1H-pyrrole-2,5-dicarboxamide, Enantiomer B

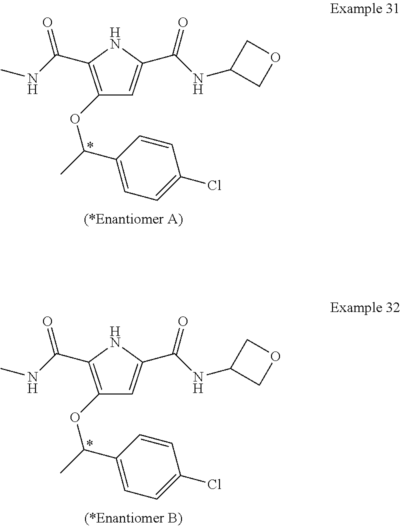 [SMILES: CNC(=O)c1[nH]c(C(=O)NC2COC2)cc1OC(C)c1ccc(Cl)cc1 ; CNC(=O)c1[nH]c(C(=O)NC2COC2)cc1OC(C)c1ccc(Cl)cc1]

Following the procedure in Example 25/Example 26, Preparation 47 using 4-(1-(4-chloro phenyl) ethoxy)-5-(methylcarbamoyl)-1H-pyrrole-2-carboxylic acid and oxetan-3-amine to give mixture of enantiomers as white solid. The mixture of enantiomers was separated by Chiral Prep HPLC² to afford Enantiomer A (0.040 g, 15%) as an off-white solid and Enantiomer B (0.049 g, 19%) as an off-white solid.

Example 31 (Enantiomer A): ¹H NMR: (400 MHz, DMSO) δ 11.35 (s, 1H), 8.99 (d, J=6.4 Hz, 1H), 7.48 (d, J=8.4 Hz, 2H), 7.41 (d, J=8.4 Hz, 2H), 7.24-7.23 (m, 1H), 6.37 (s, 1H), 5.40 (q, J=6.4 Hz, 1H), 4.90-4.83 (m, 1H), 4.76-4.72 (m, 2H), 4.48-4.42 (m, 2H), 2.86 (d, J=4.4 Hz, 3H), 1.59 (d, J=6.4 Hz, 3H). LCMS¹: m/z=378 [M+H]⁺. Chiral HPLC³ t_{R}: 2.85 min, ee %, 100%. The absolute configuration has not yet been assigned unambiguously but is believed to be the S enantiomer.

Example 32 (Enantiomer B): ¹H NMR: (400 MHz, DMSO) δ 11.34 (s, 1H), 8.89 (d, J=6.4 Hz, 1H), 7.48 (d, J=8.4 Hz, 2H), 7.41 (d, J=8.4 Hz, 2H), 7.24-7.23 (m, 1H), 6.36 (s, 1H), 5.40 (q, J=6.4 Hz, 1H), 4.90-4.83 (m, 1H), 4.76-4.72 (m, 2H), 4.48-4.42 (m, 2H), 2.86 (d, J=4.4 Hz, 3H), 1.59 (d, J=6.4 Hz, 3H). LCMS¹: m/z=378 [M+H]⁺. Chiral HPLC³ t_{R}: 3.95 min, ee %: 98.79%. The absolute configuration has not yet been assigned unambiguously but is believed to be the R enantiomer.

### Example 33—3-(1-(5-chloropyridin-2-yl) ethoxy)-N⁵-cyclobutyl-N²-methyl-1H-pyrrole-2, 5-dicarboxamide, Enantiomer A

### Example 34—3-(1-(5-chloropyridin-2-yl) ethoxy)-N⁵-cyclobutyl-N²-methyl-1H-pyrrole-2, 5-dicarboxamide, Enantiomer B

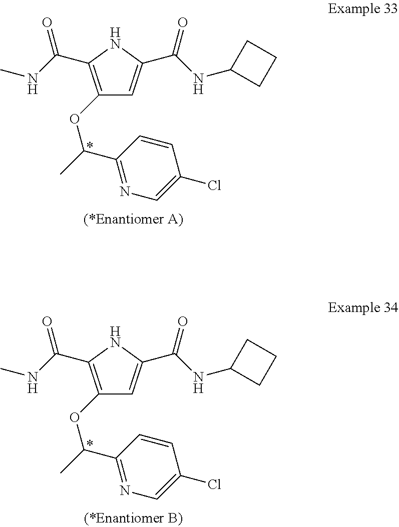 [SMILES: CNC(=O)c1[nH]c(C(=O)NC2CCC2)cc1OC(C)c1ccc(Cl)cn1 ; CNC(=O)c1[nH]c(C(=O)NC2CCC2)cc1OC(C)c1ccc(Cl)cn1]

### Preparation 51: 1-(5-chloropyridin-2-yl) ethan-1-ol

Following the procedure in Example 10, Preparation 19, using 1-(5-chloropyridin-2-yl) ethan-1-one to give title compound as colourless liquid (3.8 g, 75%). ¹H NMR: (400 MHz, DMSO) δ 8.51-8.48 (m, 1H), 7.91-7.89 (m, 1H), 7.53 (d, J=8.4 Hz, 1H), 5.48 (d, J=4.4 Hz, 1H), 4.75-4.69 (m, 1H), 1.34 (d, J=6.8 Hz, 3H). LCMS¹: m/z=158 [M+H]⁺.

### Preparation 52: Ethyl 4-(1-(5-chloropyridin-2-yl) ethoxy)-5-(methylcarbamoyl)-1H-pyrrole-2-carboxylate

Following the procedure in Example 27/Example 28, Preparation 48 using 1-(5-chloropyridin-2-yl) ethan-1-ol to give title product as a yellow gummy solid (0.80 g, 24%). LCMS¹: m/z=352 [M+H]⁺.

### Preparation 90: 4-(1-(5-chloropyridin-2-yl) ethoxy)-5-(methylcarbamoyl)-1H-pyrrole-2-carboxylic acid

Following the procedure in Example 25/Example 26, Preparation 46, using ethyl 4-(1-(5-chloropyridin-2-yl) ethoxy)-5-(methylcarbamoyl)-1H-pyrrole-2-carboxylate to give title product as an off-white solid. (0.58 g, 78%). LCMS¹: m/z=323 [M+H]⁺.

### Preparation 53: 3-(1-(5-chloropyridin-2-yl) ethoxy)-N⁵-cyclobutyl-N²-methyl-1H-pyrrole-2, 5-dicarboxamide

Following the procedure in Example 25/Example 26, Preparation 47 using 4-(1-(5-chloropyridin-2-yl) ethoxy)-5-(methylcarbamoyl)-1H-pyrrole-2-carboxylic acid and cyclobutylamine to afford mixture of enantiomers as white solid (0.35 g, 76%). The mixture of enantiomers was separated by Chiral Prep-HPLC² to afford Enantiomer A (0.12 g, 25%) as a white solid and Enantiomer B (0.11 g, 24%) as a white solid.

Example 33 (Enantiomer A): ¹H NMR: (400 MHz, DMSO) δ 11.30 (s, 1H), 8.62 (d, J=2.0 Hz, 1H), 8.43 (d, J=7.2 Hz, 1H), 7.95 (dd, J=8.8, 2.8 Hz, 1H), 7.53 (d, J=8.4 Hz, 1H), 7.38 (d, J=4.8 Hz, 1H), 6.31 (s, 1H), 5.38 (q, J=6.4 Hz, 1H), 4.26 (q, J=8.0 Hz, 1H), 2.85 (d, J=4.8 Hz, 3H), 2.20-2.15 (m, 2H), 1.99-1.87 (m, 2H), 1.71-1.68 (m, 5H). LCMS¹: m/z=377 [M+H]⁺. Chiral HPLC³ t_{R}: 3.04 min, ee %: 100%. The absolute configuration has not yet been assigned unambiguously but is believed to be the S enantiomer.

Example 34 (Enantiomer B): ¹H NMR: (400 MHz, DMSO) δ 11.30 (s, 1H), 8.62 (d, J=2.0 Hz, 1H), 8.43 (d, J=7.2 Hz, 1H), 7.95 (dd, J=8.8, 2.8 Hz, 1H), 7.53 (d, J=8.4 Hz, 1H), 7.38 (d, J=4.8 Hz, 1H), 6.31 (s, 1H), 5.38 (q, J=6.4 Hz, 1H), 4.26 (q, J=8.0 Hz, 1H), 2.85 (d, J=4.8 Hz, 3H), 2.20-2.15 (m, 2H), 1.99-1.87 (m, 2H), 1.71-1.68 (m, 5H). LCMS¹: m/z=377 [M+H]⁺. Chiral HPLC³ t_{R}: 4.57 min, ee %: 99.31%. The absolute configuration has not yet been assigned unambiguously but is believed to be the R enantiomer.

### Example 35—3-(1-(4-chloro-2-fluorophenyl) ethoxy)-N⁵-cyclobutyl-N⁵-methyl-1H-pyrrole-2,5-dicarboxamide, Enantiomer A

### Example 36—3-(1-(4-chloro-2-fluorophenyl) ethoxy)-N⁵-cyclobutyl-N²-methyl-1H-pyrrole-2,5-dicarboxamide, Enantiomer B

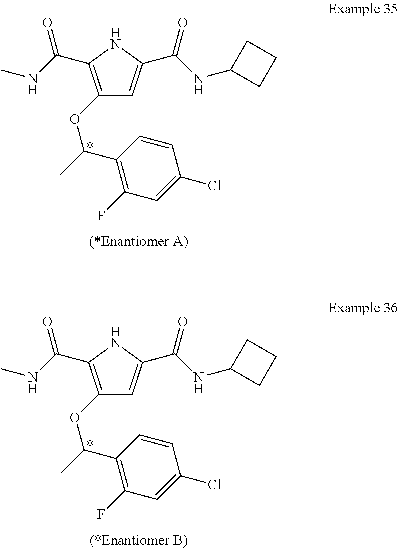 [SMILES: CNC(=O)c1[nH]c(C(=O)NC2CCC2)cc1OC(C)c1ccc(Cl)cc1F ; CNC(=O)c1[nH]c(C(=O)NC2CCC2)cc1OC(C)c1ccc(Cl)cc1F]

Following the procedure in Example 25/Example 26, Preparation 47 using 4-(1-(4-chloro-2-fluorophenyl) ethoxy)-5-(methylcarbamoyl)-1H-pyrrole-2-carboxylic acid and cyclobutylamine give racemic material as a white solid (0.30 g, 86%). The mixture of enantiomers was separated by Chiral Prep-HPLC⁷ to afford Enantiomer A (0.075 g, 21%) as an off-white solid and Enantiomer B (0.085 g, 24%) as an off-white solid.

Example 35 (Enantiomer A): ¹H NMR: (400 MHz, DMSO) δ 11.32 (s, 1H), 8.44 (d, J=7.2 Hz, 1H), 7.57 (t, J=8.4 Hz, 1H), 7.46 (dd, J=10.4, 2 Hz, 1H), 7.31 (dd, J=8.4, 2 Hz, 1H), 7.22 (d, J=4.8 Hz, 1H), 6.31 (s, 1H), 5.55 (q, J=6.4 Hz, 1H), 4.32-4.22 (m, 1H) 2.83 (d, J=4.0 Hz, 3H), 2.21-2.15 (m, 2H), 1.97-1.85 (m, 2H), 1.71-1.60 (m, 5H). LCMS¹: m/z=394 [M+H]⁺. Chiral HPLC³ t_{R}: 2.32 min, ee %: 100%. The absolute configuration has not yet been assigned unambiguously but is believed to be the S enantiomer.

Example 36 (Enantiomer B): ¹H NMR: (400 MHz, DMSO) δ 11.32 (s, 1H), 8.43 (d, J=7.2 Hz, 1H), 7.57 (t, J=8.4 Hz, 1H), 7.46 (dd, J=10.4, 1.6 Hz, 1H), 7.31 (m, 1H), 7.22 (d, J=4.8 Hz, 1H), 6.31 (s, 1H), 5.55 (q, J=6.4 Hz, 1H), 4.27 (q, J=8.0 Hz, 1H), 2.85 (d, J=4.4 Hz, 3H), 2.20-2.15 (m, 2H), 1.95-1.88 (m, 2H), 1.69-1.62 (m, 5H). LCMS¹: m/z=394 [M+H]⁺. Chiral HPLC³ t_{R}: 3.64 min, ee %: 100%. The absolute configuration has not yet been assigned unambiguously but is believed to be the R enantiomer.

### Example 37—3-(1-(4-chloro-2-fluorophenyl) ethoxy)-N⁵-(trans-4-hydroxycyclohexyl)-N²-methyl-1H-pyrrole-2,5-dicarboxamide, Enantiomer A

### Example 38—3-(1-(4-chloro-2-fluorophenyl) ethoxy)-N⁵-(trans-4-hydroxycyclohexyl)-N²-methyl-1H-pyrrole-2,5-dicarboxamide, Enantiomer B

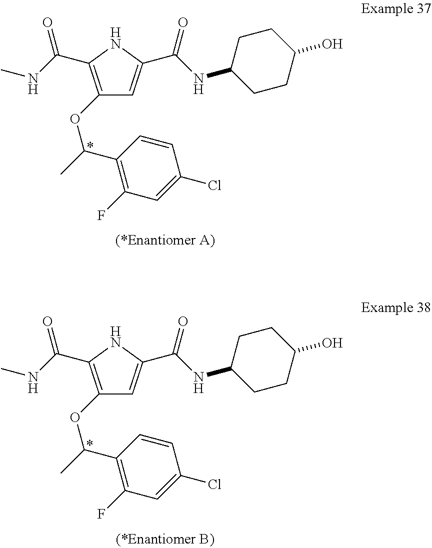

Following the procedure in Example 25/Example 26, Preparation 47 using 4-(1-(4-chloro-2-fluorophenyl) ethoxy)-5-(methylcarbamoyl)-1H-pyrrole-2-carboxylic acid and trans-4-aminocyclohexan-1-ol to give a white solid as mixture of enantiomers (0.25 g, 64%). The mixture of enantiomers was separated by Chiral Prep-HPLC⁶ to afford Enantiomer A (0.065 g, 16%) as an off-white solid and Enantiomer B (0.06 g, 15%) as an off-white solid.

Example 37 (Enantiomer A): ¹H NMR: (400 MHz, DMSO) δ 11.35 (s, 1H), 8.04 (d, J=7.6 Hz, 1H), 7.56 (t, J=8.0 Hz, 1H), 7.46 (dd, J=10.4, 1.6 Hz, 1H), 7.31 (dd, J=8.4, 2.0 Hz, 1H), 7.21 (d, J=4.8 Hz, 1H), 6.32 (s, 1H), 5.56 (q, J=6.4 Hz, 1H), 4.55 (d, J=4.0 Hz, 1H), 3.57 (br s, 1H), 3.36 (d, J=6.8 Hz, 1H), 2.84 (d, J=4.8 Hz, 3H), 1.80 (m, 4H), 1.62 (d, J=6.4 Hz, 3H), 1.22-1.18 (m, 4H). LCMS¹: m/z=438 [M+H]⁺. Chiral HPLC⁷ t_{R}: 3.81 min, ee %: 96.67%. The absolute configuration has not yet been assigned unambiguously but is believed to be the R enantiomer.

Example 38 (Enantiomer B): ¹H NMR: (400 MHz, DMSO) δ 11.35 (s, 1H), 8.04 (d, J=7.6 Hz, 1H), 7.56 (t, J=8.0 Hz, 1H), 7.46 (dd, J=10.4, 2.0 Hz, 1H) 7.31 (dd, J=8.4, 2 Hz, 1H), 7.23-7.19 (m, 1H), 6.31 (s, 1H), 5.56 (q, J=6.0 Hz, 1H), 4.55 (d, J=4.0 Hz, 1H), 3.57 (br s, 1H), 3.36 (d, J=6.8 Hz, 1H), 2.84 (d, J=4.8 Hz, 3H), 1.80 (m, 4H), 1.62 (d, J=6.4 Hz, 3H), 1.22-1.18 (m, 4H). LCMS¹: m/z=438 [M+H]⁺. Chiral HPLC⁷ t_{R}: 4.37 min, ee %: 96.21%. The absolute configuration has not yet been assigned unambiguously but is believed to be the S enantiomer.

### Example 39—3-(1-(4-chloro-2-fluorophenyl) ethoxy)-N⁵-methyl-N²-(oxetan-3-yl)-1H-pyrrole-2,5-dicarboxamide, Enantiomer A

### Example 40—3-(1-(4-chloro-2-fluorophenyl) ethoxy)-N⁵-methyl-N²-(oxetan-3-yl)-1H-pyrrole-2,5-dicarboxamide, Enantiomer B

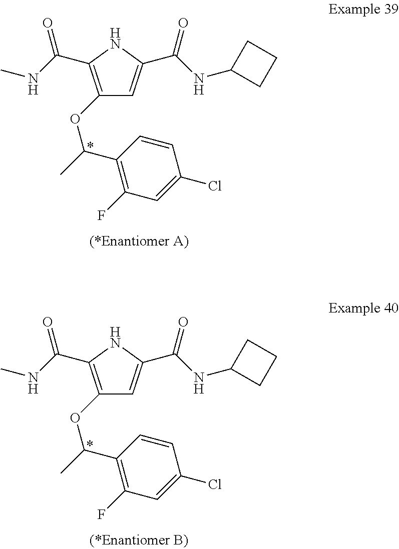 [SMILES: CNC(=O)c1[nH]c(C(=O)NC2CCC2)cc1OC(C)c1ccc(Cl)cc1F ; CNC(=O)c1[nH]c(C(=O)NC2CCC2)cc1OC(C)c1ccc(Cl)cc1F]

Following the procedure in Example 25/Example 26, Preparation 47 using 4-(1-(4-chloro-2-fluorophenyl) ethoxy)-5-(methylcarbamoyl)-1H-pyrrole-2-carboxylic acid and oxetan-3-amine to give a white solid as mixture of enantiomers (0.25 g, 71%). The mixture of enantiomers was separated by Chiral Prep-HPLC⁴ to afford Enantiomer A (0.062 g, 17%) as an off-white solid and Enantiomer B (0.032 g, 9%) as an off-white solid.

Example 39 (Enantiomer A): ¹H NMR: (400 MHz, DMSO) δ 11.38 (s, 1H), 8.90 (d, J=6.4 Hz, 1H), 7.57 (t, J=8.0 Hz, 1H), 7.46 (dd, J=10.4, 2.0 Hz, 1H), 7.31 (dd, J=8.4, 1.6 Hz, 1H), 7.25 (d, J=4.8 Hz, 1H), 6.37 (s, 1H), 5.56 (q, J=6.4 Hz, 1H), 4.91-4.85 (m, 1H), 4.76-4.72 (m, 2H), 4.48-4.43 (m, 2H), 2.85 (d, J=4.8 Hz, 3H), 1.62 (d, J=6.4 Hz, 3H). LCMS¹: m/z=396 [M+H]⁺. Chiral HPLC³ t_{R}: 2.71 min, ee %: 100%. The absolute configuration has not yet been assigned unambiguously but is believed to be the S enantiomer.

Example 40 (Enantiomer B): ¹H NMR: (400 MHz, DMSO) δ 10.85 (br s, 1H), 8.87 (br s, 1H), 7.57 (t, J=8.0 Hz, 1H), 7.46 (dd, J=10.4, 2.0 Hz, 1H), 7.31 (dd, J=8.4, 1.6 Hz, 1H), 7.25 (d, J=4.8 Hz, 1H), 6.37 (s, 1H), 5.56 (q, J=6.4 Hz, 1H), 4.91-4.85 (m, 1H), 4.76-4.72 (m, 2H), 4.48-4.43 (m, 2H), 2.85 (d, J=4.8 Hz, 3H), 1.62 (d, J=6.4 Hz, 3H). LCMS¹: m/z=396 [M+H]⁺. Chiral HPLC³ t_{R}: 4.58 min, ee %: 96.16%. The absolute configuration has not yet been assigned unambiguously but is believed to be the R enantiomer.

### Example 41—3-(1-(4-chloro-2-fluorophenyl) ethoxy)-N⁵-ethyl-N²-methyl-1H-pyrrole-2,5-dicarboxamide, Enantiomer A

### Example 42—3-(1-(4-chloro-2-fluorophenyl) ethoxy)-N⁵-ethyl-N²-methyl-1H-pyrrole-2,5-dicarboxamide, Enantiomer B

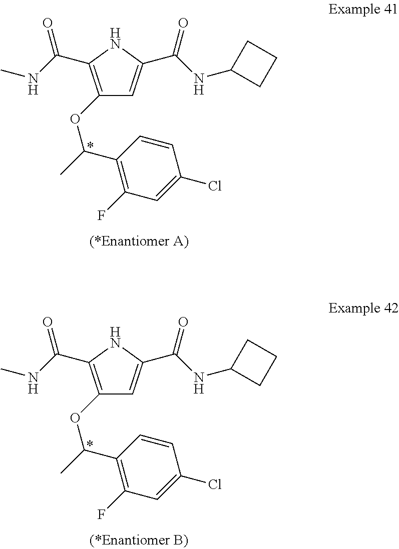 [SMILES: CNC(=O)c1[nH]c(C(=O)NC2CCC2)cc1OC(C)c1ccc(Cl)cc1F ; CNC(=O)c1[nH]c(C(=O)NC2CCC2)cc1OC(C)c1ccc(Cl)cc1F]

### Preparation 54: 1-(4-chloro-2-fluorophenyl) ethan-1-ol

Following the procedure in Example 10, Preparation 19, 1-(4-chloro-2-fluorophenyl) ethan-1-one (5 g, 29.06 mmol) was reacted to give title compound as off-white solid (5.0 g, 88%). ¹H NMR: (400 MHz, DMSO) δ 7.53 (t, J=8.4 Hz, 1H), 7.35-7.20 (m, 2H), 5.40 (d, J=4.4 Hz, 1H), 4.94 (m, 1H), 1.31 (d, J=6.4 Hz, 3H).

### Preparation 55: Ethyl 4-(1-(4-chloro-2-fluorophenyl)ethoxy)-5-(methylcarbamoyl)-1H-pyrrole-2-carboxylate

Following the procedure in Example 25/Example 26, Preparation 45, ethyl 4-hydroxy-5-(methylcarbamoyl)-1H-pyrrole-2-carboxylate and 1-(4-chloro-2-fluorophenyl) ethan-1-ol were reacted to give title compound as yellow gummy solid (3.8 g, 58%). ¹H NMR: (400 MHz, DMSO) δ 11.73 (s, 1H), 8.62 (s, 1H), 7.61-7.45 (m, 1H), 7.36-7.31 (m, 2H), 6.41 (s, 1H), 5.61 (q, J=6.4 Hz, 1H), 4.03 (q, J=7.2 Hz, 2H), 2.83 (d, J=4.4 Hz, 3H), 1.62 (d, J=6.4 Hz, 3H), 1.29 (t, J=7.2 Hz, 3H). LCMS¹: m/z=369 [M+H]⁺.

### Preparation 56: 4-(1-(4-chloro-2-fluorophenyl) ethoxy)-5-(methylcarbamoyl)-1H-pyrrole-2-carboxylic acid

Following the procedure in Example 25/Example 26, Preparation 46, ethyl 4-(1-(4-chloro-2-fluorophenyl)ethoxy)-5-(methylcarbamoyl)-1H-pyrrole-2-carboxylate was reacted to give title compound as pink solid. (1.8 g, 51%). ¹H NMR: 1H NMR (400 MHz, DMSO) δ 12.60 (s, 1H), 11.44 (s, 1H), 7.59 (t, J=8.0 Hz, 1H), 7.46 (dd, J=10.4, 1.6 Hz, 1H), 7.35-7.30 (m, 2H), 6.33 (s, 1H), 5.58 (q, J=6.4 Hz, 1H), 2.82 (d, J=4.4, 3H), 1.62 (d, J=6.4 Hz, 3H).

LCMS¹: m/z=341 [M+H]⁺.

### Preparation 57: 3-(1-(4-chloro-2-fluorophenyl) ethoxy)-N5-ethyl-N2-methyl-1H-pyrrole-2,5-dicarboxamide

Following the procedure in Example 25/Example 26, Preparation 47, 4-(1-(4-chloro-2-fluorophenyl) ethoxy)-5-(methylcarbamoyl)-1H-pyrrole-2-carboxylic acid ethylamine (2 M in THF) were reacted to give racemic material as a white solid (0.20 g, 61%). The mixture of enantiomers was separated by Chiral Prep-HPLC¹ to afford Enantiomer A (0.06 g, 18%) as an off-white solid and Enantiomer B (0.05 g, 15%) as an off-white solid.

Example 41 (Enantiomer A): ¹H NMR: (400 MHz, DMSO) δ 11.26 (s, 1H), 8.22 (t, J=5.2 Hz, 1H), 7.57 (t, J=8.4 Hz, 1H), 7.46 (dd, J=10.4, 2 Hz, 1H), 7.31 (dd, J=8, 1.6 Hz, 1H), 7.22 (d, J=4.8 Hz, 1H), 6.32 (s, 1H), 5.55 (q, J=6.4 Hz, 1H), 3.20-3.16 (m, 2H), 2.84 (d, J=4.8 Hz, 3H) 1.63 (d, J=6.4 Hz, 3H), 1.06 (t, J=7.2 Hz, 3H). LCMS¹: m/z=368 [M+H]⁺. Chiral HPLC³ t_{R}: 2.05 min, ee %: 100%. The absolute configuration has not yet been assigned unambiguously but is believed to be the S enantiomer.

Example 42 (Enantiomer B): ¹H NMR: (400 MHz, DMSO) δ 11.26 (s, 1H), 8.22 (t, J=5.2 Hz, 1H), 7.57 (t, J=8.4 Hz, 1H), 7.46 (dd, J=10.4, 1.6 Hz, 1H), 7.31 (dd, J=8, 1.6 Hz, 1H), 7.22 (d, J=4.8 Hz, 1H), 6.32 (s, 1H), 5.55 (q, J=6.4 Hz, 1H), 3.21-3.15 (m, 2H), 2.84 (d, J=4.8 Hz, 3H) 1.63 (d, J=6.4 Hz, 3H), 1.06 (t, J=7.2 Hz, 3H). LCMS¹: m/z=368 [M+H]⁺. Chiral HPLC³ t_{R}: 2.51 min, ee %: 99.11%. The absolute configuration has not yet been assigned unambiguously but is believed to be the R enantiomer.

### Example 43—N⁵-cyclobutyl-3-(1-(2-fluorophenyl) ethoxy)-N²-methyl-1H-pyrrole-2,5-dicarboxamide, Enantiomer A

### Example 44—N⁵-cyclobutyl-3-(1-(2-fluorophenyl) ethoxy)-N²-methyl-1H-pyrrole-2,5-dicarboxamide, Enantiomer B

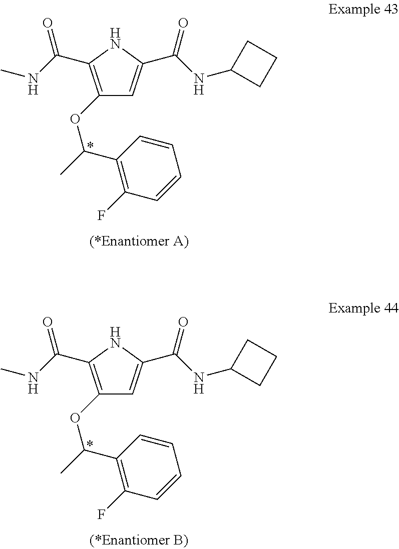 [SMILES: CNC(=O)c1[nH]c(C(=O)NC2CCC2)cc1OC(C)c1ccccc1F ; CNC(=O)c1[nH]c(C(=O)NC2CCC2)cc1OC(C)c1ccccc1F]

### Preparation 58: Ethyl 4-(1-(2-fluorophenyl)ethoxy)-5-(methylcarbamoyl)-1H-pyrrole-2-carboxylate

Following the procedure in Example 25/Example 26, Preparation 45, ethyl 4-hydroxy-5-(methylcarbamoyl)-1H-pyrrole-2-carboxylate and 1-(2-fluorophenyl) ethan-1-ol were reacted to give title compound as yellow gummy solid (3.5 g, 88%). LCMS¹: m/z=335 [M+H]⁺.

### Preparation 59: 4-(1-(2-fluoro phenyl) ethoxy)-5-(methylcarbamoyl)-1H-pyrrole-2-carboxylic acid

Following the procedure in Example 25/Example 26, Preparation 46, ethyl 4-(1-(2-fluorophenyl)ethoxy)-5-(methylcarbamoyl)-1H-pyrrole-2-carboxylate was reacted to give title compound as pink solid. (1.9 g, 60%). ¹H NMR: (400 MHz, DMSO) δ 12.65 (br s, 1H), 11.41 (s, 1H), 7.55 (q, J=6 Hz, 1H), 7.35-7.32 (m, 2H), 7.23-7.19 (m, 2H), 6.33 (s, 1H), 5.60 (q, J=6 Hz, 1H), 2.83 (d, J=4.8 Hz, 3H), 1.63 (d, J=6.4 Hz, 3H). LCMS¹: m/z=307 [M+H]⁺.

### Preparation 60: N⁵-cyclobutyl-3-(1-(2-fluorophenyl) ethoxy)-N²-methyl-1H-pyrrole-2,5-dicarboxamide

Following the procedure in Example 25/Example 26, Preparation 47, 4-(1-(2-fluoro phenyl) ethoxy)-5-(methylcarbamoyl)-1H-pyrrole-2-carboxylic acid and cyclobutylamine were reacted to give off-white solid as mixture of enantiomers (0.36 g, 76%). The mixture of enantiomers was separated by Chiral Prep-HPLC⁴ to afford Enantiomer A (0.102 g, 21%) as a white solid and Enantiomer B (0.109 g, 23%) as a white solid.

Example 43 (Enantiomer A): ¹H NMR: (400 MHz, DMSO) δ 11.30 (s, 1H), 8.43 (d, J=7.2 Hz, 1H), 7.53 (q, J=6 Hz, 1H), 7.35-7.31 (m, 1H), 7.23-7.18 (m, 3H), 6.32 (s, 1H), 5.57 (q, J=6.4 Hz, 1H), 4.26 (q, J=8 Hz, 1H), 2.86 (d, J=4.4 Hz, 3H), 2.19-2.15 (m, 2H), 1.95-1.87 (m, 2H), 1.68-1.62 (m, 5H). LCMS¹: m/z=360 [M+H]⁺. Chiral HPLC³ t_{R}: 2.23 min, ee %: 100%. The absolute configuration has not yet been assigned unambiguously but is believed to be the S enantiomer.

Example 44 (Enantiomer B): ¹H NMR: (400 MHz, DMSO-d⁶) δ 11.30 (s, 1H), 8.44 (d, J=7.2 Hz, 1H), 7.53 (q, J=6 Hz, 1H), 7.35-7.31 (m, 1H), 7.23-7.18 (m, 3H), 6.32 (s, 1H), 5.58-5.57 (m, 1H), 4.26 (q, J=8 Hz, 1H), 2.86 (d, J=4.8 Hz, 3H), 2.18 (t, J=2.4 HZ, 2H), 1.91 (q, J=9.6 Hz, 2H), 1.68-1.62 (m, 5H). LCMS¹: m/z=360 [M+H]⁺. Chiral HPLC³ t_{R}: 2.66 min, ee %: 97.91%. The absolute configuration has not yet been assigned unambiguously but is believed to be the R enantiomer.

### Example 45—3-(1-(2-fluorophenyl) ethoxy)-N⁵-(trans-4-hydroxycyclohexyl)-N²-methyl-1H-pyrrole-2,5-dicarboxamide, Enantiomer A

### Example 46—3-(1-(2-fluorophenyl) ethoxy)-N⁵-(trans-4-hydroxycyclohexyl)-N²-methyl-1H-pyrrole-2,5-dicarboxamide, Enantiomer B

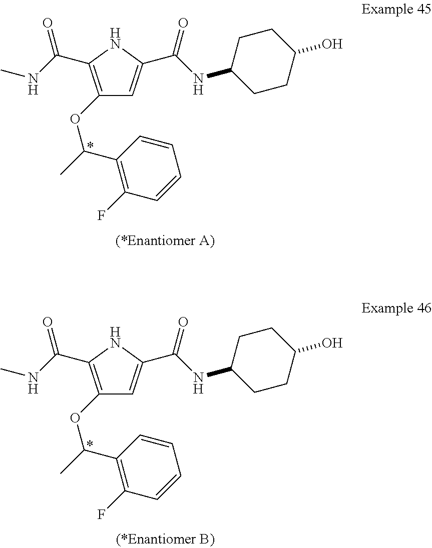

Following the procedure in Example 25/Example 26, Preparation 47, 4-(1-(2-fluoro phenyl) ethoxy)-5-(methylcarbamoyl)-1H-pyrrole-2-carboxylic acid and trans-4-aminocyclohexan-1-ol were reacted to give white solid as mixture of enantiomers (0.26 g, 65%). The mixture of enantiomers was separated by Chiral Prep-HPLC⁸ to afford Enantiomer A (0.072 g, 18%) as a white solid and Enantiomer B (0.068 g, 17%) as white solid.

Example 45 (Enantiomer A): ¹H NMR: (400 MHz, DMSO) δ 11.32 (s, 1H), 8.03 (d, J=7.6 Hz, 1H), 7.52 (t, J=7.6 Hz, 1H), 7.35-7.31 (m, 1H), 7.23-7.18 (m, 3H), 6.32 (s, 1H), 5.58 (q, J=6.4 Hz, 1H), 4.56 (d, J=4.4 Hz, 1H), 3.57 (br s, 1H), 3.37 (s, 1H), 2.85 (d, J=4.8 Hz, 3H), 1.79 (br s, 4H), 1.64 (d, J=6.4 Hz, 3H), 1.23-1.17 (m, 4H). LCMS¹: m/z=404 [M+H]⁺. Chiral HPLC⁹ t_{R}: 4.28 min, ee %: 100%. The absolute configuration has not yet been assigned unambiguously but is believed to be the S enantiomer.

Example 46 (Enantiomer B): ¹H NMR: (400 MHz, DMSO) δ 11.32 (s, 1H), 8.03 (d, J=7.6 Hz, 1H), 7.52 (t, J=7.6 Hz, 1H), 7.35-7.32 (m, 1H), 7.23-7.18 (m, 3H), 6.32 (s, 1H), 5.58 (q, J=6.4 Hz, 1H), 4.56 (d, J=4 Hz, 1H), 3.57 (br s, 1H), 3.37 (s, 1H), 2.85 (d, J=4.8 Hz, 3H), 1.80 (d, J=4.4 Hz, 4H), 1.64 (d, J=6.4 Hz, 3H), 1.23-1.15 (m, 4H). LCMS¹: m/z=404 [M+H]⁺. Chiral HPLC⁹ t_{R}: 4.71 min, ee %: 97.84%. The absolute configuration has not yet been assigned unambiguously but is believed to be the R enantiomer.

### Example 47—3-(1-(2-fluorophenyl) ethoxy)-N⁵-methyl-N²-(oxetan-3-yl)-1H-pyrrole-2,5-dicarboxamide, Enantiomer A

### Example 48—3-(1-(2-fluorophenyl) ethoxy)-N⁵-methyl-N²-(oxetan-3-yl)-1H-pyrrole-2,5-dicarboxamide, Enantiomer B

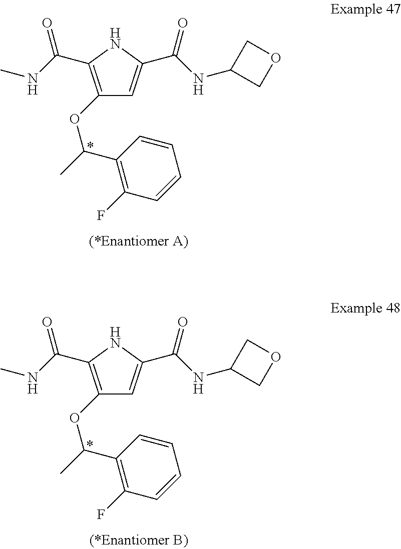 [SMILES: CNC(=O)c1[nH]c(C(=O)NC2COC2)cc1OC(C)c1ccccc1F ; CNC(=O)c1[nH]c(C(=O)NC2COC2)cc1OC(C)c1ccccc1F]

Following the procedure in Example 25/Example 26, Preparation 47, 4-(1-(2-fluoro phenyl) ethoxy)-5-(methylcarbamoyl)-1H-pyrrole-2-carboxylic acid and oxetan-3-amine were reacted to give an off-white solid as mixture of enantiomers (0.30 g, 63%). The mixture of enantiomers was separated by Chiral Prep-HPLC² to afford Enantiomer A (0.100 g, 21%) as an off-white solid and Enantiomer B (0.108 g, 22%) as an off-white solid.

Example 47 (Enantiomer A): ¹H NMR: (400 MHz, DMSO) δ 11.38 (s, 1H), 8.90 (d, J=6 Hz, 1H), 7.55-7.52 (m, 1H), 7.35-7.32 (m, 1H), 7.26-7.18 (m, 3H), 6.37 (s, 1H), 5.59-5.58 (m, 1H), 4.88-4.85 (m, 1H), 4.76-4.71 (m, 2H), 4.48-4.43 (m, 2H), 2.86 (d, J=4.4 Hz, 3H), 1.64 (d, J=6.4 Hz, 3H). LCMS¹: m/z=362 [M+H]⁺. Chiral HPLC³ t_{R}: 2.60 min, ee %: 100%. The absolute configuration has not yet been assigned unambiguously but is believed to be the S enantiomer.

Example 48 (Enantiomer B): ¹H NMR: (400 MHz, DMSO) δ 11.39 (s, 1H), 8.90 (d, J=6 Hz, 1H), 7.55-7.52 (m, 1H), 7.37-7.31 (m, 1H), 7.26-7.18 (m, 3H), 6.37 (s, 1H), 5.58 (q, J=6 Hz, 1H), 4.88-4.85 (m, 1H), 4.76-4.71 (m, 2H), 4.48-4.43 (m, 2H), 2.86 (d, J=4.4 Hz, 3H), 1.64 (d, J=6.4 Hz, 3H). LCMS¹: m/z=362 [M+H]⁺. Chiral HPLC³ t_{R}: 4.04 min, ee %: 99.01%. The absolute configuration has not yet been assigned unambiguously but is believed to be the R enantiomer.

### Example 49—3-(1-(4-bromophenyl) ethoxy)-N⁵-ethyl-N²-methyl-1H-pyrrole-2,5-dicarboxamide, Enantiomer A

### Example 50—3-(1-(4-bromophenyl) ethoxy)-N⁵-ethyl-N²-methyl-1H-pyrrole-2,5-dicarboxamide, Enantiomer B

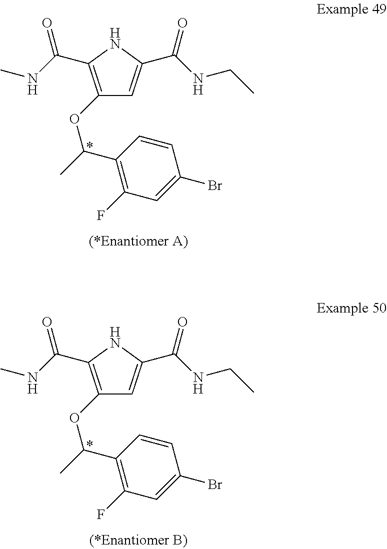

### Preparation 61: ethyl 4-(1-(4-bromophenyl) ethoxy)-5-(methyl carbamoyl)-1H-pyrrole-2-carboxylate

Following the procedure in Example 28/Example 29, Preparation 48, ethyl 4-hydroxy-5-(methyl carbamoyl)-1H-pyrrole-2-carboxylate and 1-(4-bromophenyl) ethan-1-ol were reacted to give title compound as a yellow solid (1.3 g, 35%). LCMS¹: m/z=395 [M+H]⁺.

### Preparation 62: 4-(1-(4-bromophenyl) ethoxy)-5-(methyl carbamoyl)-1H-pyrrole-2-carboxylic acid

Following the procedure in Example 25/Example 26, Preparation 46, ethyl 4-(1-(4-bromophenyl) ethoxy)-5-(methyl carbamoyl)-1H-pyrrole-2-carboxylate was reacted to give title compound as a light pink solid. (0.2 g, 20%). ¹H NMR: (400 MHz, DMSO) δ 12.50 (br s, 1H), 11.33 (s, 1H), 7.53 (d, J=2.4 Hz, 2H), 7.44 (d, J=2.4 Hz, 2H), 7.34-7.31 (m, 1H), 6.34 (d, J=2.8 Hz, 1H), 5.41 (q, J=6.4 Hz, 1H), 2.83 (d, J=4.8 Hz, 3H), 1.57 (d, J=6.4 Hz, 3H).

### Preparation 63: 3-(1-(4-bromophenyl) ethoxy)-N5-ethyl-N2-methyl-1H-pyrrole-2,5-dicarboxamide

Following the procedure in Example 25/Example 26, Preparation 47, 4-(1-(4-bromophenyl) ethoxy)-5-(methyl carbamoyl)-1H-pyrrole-2-carboxylic acid and ethylamine (2 M in THF) were reacted to give a white solid as mixture of enantiomers (0.095 g, 44%). The mixture of enantiomers was separated by Chiral Prep-HPLC⁹ to afford Enantiomer A (0.031 g, 14%) and Enantiomer B (0.038 g, 18%) as an off-white solids.

Example 49 (Enantiomer A): ¹H NMR: (400 MHz, DMSO) δ 11.22 (s, 1H), 8.20 (t, J=5.2 Hz, 1H), 7.55 (d, J=8.0 Hz, 2H), 7.41 (d, J=8.0 Hz, 2H), 7.20 (d, J=4.8 Hz, 1H), 6.31 (s, 1H), 5.38 (q, J=6.0 Hz, 1H), 3.19-3.15 (m, 2H), 2.85 (d, J=4.8 Hz, 3H), 1.58 (d, J=6.4 Hz, 3H), 1.06 (t, J=7.2 Hz, 3H). LCMS¹: m/z=394 [M+H]⁺. Chiral HPLC⁵ t_{R}: 4.09 min, ee %: 100%. The absolute configuration has not yet been assigned unambiguously but is believed to be the S enantiomer.

Example 50 (Enantiomer B): ¹H NMR: (400 MHz, DMSO) δ 11.22 (s, 1H), 8.20 (t, J=5.2 Hz, 1H), 7.55 (d, J=8.0 Hz, 2H), 7.41 (d, J=8.0 Hz, 2H), 7.20 (d, J=4.8 Hz, 1H), 6.31 (s, 1H), 5.38 (q, J=6.0 Hz, 1H), 3.19-3.14 (m, 2H), 2.85 (d, J=4.8 Hz, 3H), 1.58 (d, J=6.4 Hz, 3H), 1.06 (t, J=7.2 Hz, 3H). LCMS¹: m/z=394 [M+H]⁺. Chiral HPLC⁵ t_{R}: 4.53 min, ee %: 99.61%. The absolute configuration has not yet been assigned unambiguously but is believed to be the R enantiomer.

### Example 51—N⁵-ethyl-3-(1-(4-iodophenyl) ethoxy)-N²-methyl-1H-pyrrole-2,5-dicarboxamide, Enantiomer A

### Example 52—N⁵-ethyl-3-(1-(4-iodophenyl) ethoxy)-N²-methyl-1H-pyrrole-2,5-dicarboxamide, Enantiomer A

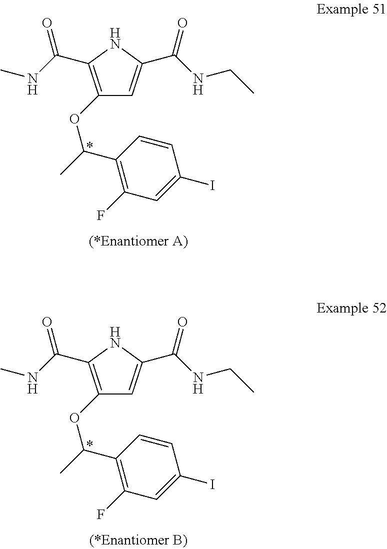

### Preparation 64: Ethyl 4-(1-(4-iodophenyl) ethoxy)-5-(methyl carbamoyl)-1H-pyrrole-2-carboxylate

Following the procedure in Example 28/Example 29, Preparation 48, ethyl 4-hydroxy-5-(methyl carbamoyl)-1H-pyrrole-2-carboxylate and 1-(4-iodophenyl) ethan-1-ol were reacted to give title compound as light-yellow solid (1.3 g, 32%) which was used directly in the next step.

### Preparation 65: 4-(1-(4-iodophenyl) ethoxy)-5-(methyl carbamoyl)-1H-pyrrole-2-carboxylic acid

Following the procedure in Example 25/Example 26, Preparation 46, ethyl 4-(1-(4-iodophenyl) ethoxy)-5-(methyl carbamoyl)-1H-pyrrole-2-carboxylate was reacted to give title compound as off-white solid (0.3 g, 24%). ¹H NMR: (400 MHz, DMSO) δ 12.50 (br s, 1H), 11.32 (s, 1H), 7.71 (d, J=8.0 Hz, 2H), 7.33-7.26 (m, 3H), 6.34 (d, J=2.8 Hz, 1H), 5.41 (q, J=6.4 Hz, 1H), 2.83 (d, J=4.8 Hz, 3H), 1.57 (d, J=6.4 Hz, 3H).

### Preparation 66: N⁵-ethyl-3-(1-(4-iodophenyl) ethoxy)-NV-methyl-1H-pyrrole-2,5-dicarboxamide

Following the procedure in Example 25/Example 26, Preparation 47, 4-(1-(4-iodophenyl) ethoxy)-5-(methyl carbamoyl)-1H-pyrrole-2-carboxylic acid and ethylamine (2 M in THF) were reacted to give a white solid as mixture of enantiomers (0.15 g, 47%). The mixture of enantiomers was separated by Chiral Prep-HPLC⁹ to afford Enantiomer A (0.058 g, 18%) and Enantiomer B (0.060 g, 19%) as an off-white solid.

Example 51 (Enantiomer A): ¹H NMR: (400 MHz, DMSO) δ 11.22 (s, 1H), 8.20 (s, 1H), 7.71 (d, J=8.0 Hz, 2H), 7.27-7.19 (m, 3H), 6.30 (d, J=2.0 Hz, 1H), 5.34 (t, J=6.0 Hz, 1H), 3.17 (s, 2H), 2.85 (d, J=4.4 Hz, 3H), 1.58 (d, J=6 Hz, 3H), 1.06 (t, J=7.2 Hz, 3H). LCMS¹: m/z=442 [M+H]⁺. Chiral HPLC³ t_{R}: 3.18 min, ee %: 100%. The absolute configuration has not yet been assigned unambiguously but is believed to be the S enantiomer.

Example 52 (Enantiomer B): ¹H NMR: (400 MHz, DMSO-d⁶) δ 11.22 (s, 1H), 8.19 (t, J=5.2 Hz, 1H), 7.71 (d, J=8.4 Hz, 2), 7.26 (d, J=8.4 Hz, 2H), 7.20 (d, J=4.8 Hz, 1H), 6.30 (d, J=2.8 Hz, 1H), 5.34 (d, J=6.4 Hz, 1H), 3.17 (brs, 2H), 2.85 (d, J=4.8 Hz, 3H), 1.57 (d, J=6.4 Hz, 3H), 1.06 (t, J=7.6 Hz, 3H). LCMS¹: m/z=442 [M+H]⁺. Chiral HPLC³ t_{R}: 3.50 min, ee %: 100%. The absolute configuration has not yet been assigned unambiguously but is believed to be the R enantiomer.

### Example 53—3-(benzhydryloxy)-N⁵-ethyl-N²-methyl-1H-pyrrole-2,5-dicarboxamide

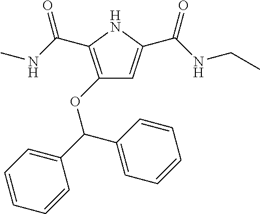 [SMILES: CCNC(=O)c1cc(OC(c2ccccc2)c2ccccc2)c(C(=O)NC)[nH]1]

### Preparation 67: Diphenylmethanol

Following the procedure in Example 10, Preparation 19, benzophenone was reacted to give title compound as off-white solid (5.0 g, 98%). ¹H NMR: (400 MHz, DMSO-d⁶) δ 7.44-7.37 (m, 4H), 7.30 (t, J=7.2 Hz, 4H), 7.20 (t, J=7.2 Hz, 2H), 5.90 (br s, 1H), 5.70 (s, 1H).

### Preparation 68: Ethyl 4-(benzhydryloxy)-5-(methylcarbamoyl)-1H-pyrrole-2-carboxylate

Following the procedure in Example 25/Example 26, Preparation 45, ethyl 4-hydroxy-5-(methylcarbamoyl)-1H-pyrrole-2-carboxylate and diphenylmethanol were reacted to give title compound as yellow gummy solid (0.52 g, 47%). LCMS¹: m/z=379 [M+H]⁺.

### Preparation 69: 4-(benzhydryloxy)-5-(methylcarbamoyl)-1H-pyrrole-2-carboxylic acid

Following the procedure in Example 25/Example 26, Preparation 46, ethyl 4-(benzhydryloxy)-5-(methylcarbamoyl)-1H-pyrrole-2-carboxylate was reacted to give title compound as pink solid (0.29 g, 62%). LCMS¹: m/z=351 [M+H]⁺.

### Preparation 70: 3-(benzhydryloxy)-N5-ethyl-N2-methyl-1H-pyrrole-2,5-dicarboxamide

Following the procedure in Example 25/Example 26, Preparation 47, 4-(benzhydryloxy)-5-(methylcarbamoyl)-1H-pyrrole-2-carboxylic acid and ethylamine were reacted to give title compound as off-white solid (0.09 g, 32%). ¹H NMR: (400 MHz, DMSO-d⁶) δ 11.35 (s, 1H), 8.22 (t, J=4.8 Hz, 1H), 7.54 (d, J=7.6 Hz, 4H), 7.37 (t, J=7.6 Hz, 4H), 7.29 (t, J=7.2 Hz, 2H), 7.18 (d, J=4.8 Hz, 1H), 6.45-6.42 (m, 2H), 3.21-3.15 (m, 2H), 2.85 (d, J=4.8 Hz, 3H), 1.08 (t, J=4 Hz, 3H). LCMS¹: m/z=376 [M−H]⁻.

### Example 54—(S)—N⁵-methyl-3-(1-phenylethoxy)-N⁵-(2,2,2-trifluoroethyl)-1H-pyrrole-2,5-dicarboxamide

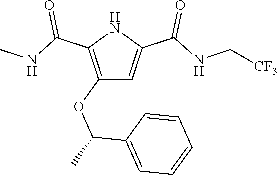 [SMILES: CNC(=O)c1[nH]c(C(=O)NCC(F)(F)F)cc1O[C@@H](C)c1ccccc1]

Following the procedure in Example 108, using 2,2,2-trifluoroethan-1-amine to give title compound as a white solid (0.030 g, 15%). ¹H NMR: (400 MHz, DMSO) δ 11.46 (s, 1H), 8.80 (t, J=6.0 Hz, 1H), 7.45 (d, J=7.6 Hz, 2H), 7.35 (t, J=7.6 Hz, 2H), 7.26-7.25 (m, 2H), 6.45 (s, 1H), 5.39 (q, J=6.0 Hz, 1H), 4.08-3.98 (m, 2H), 2.86 (d, J=4.4 Hz, 3H), 1.61 (d, J=6.4 Hz, 3H). LCMS¹: m/z=370 [M+H]⁺.

### Example 55—3-(1-(2, 6-difluorophenyl) ethoxy)-N⁵-ethyl-N²-methyl-1H-pyrrole-2, 5-dicarboxamide, Enantiomer A

### Example 56—3-(1-(2, 6-difluorophenyl) ethoxy)-N⁵-ethyl-N²-methyl-1H-pyrrole-2, 5-dicarboxamide, Enantiomer B

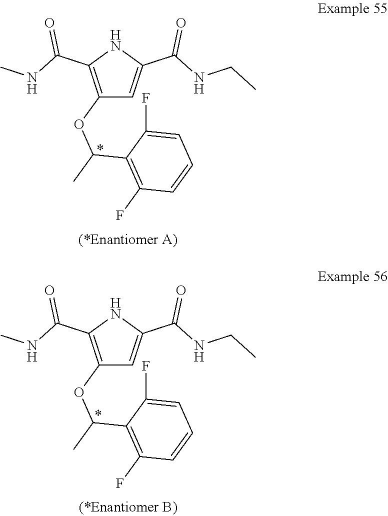

### Preparation 71: Ethyl 4-(1-(2, 6-difluorophenyl) ethoxy)-5-(methylcarbamoyl)-1H-pyrrole-2-carboxylate

Following the procedure in Example 25/Example 26, Preparation 45, ethyl 4-hydroxy-5-(methylcarbamoyl)-1H-pyrrole-2-carboxylate and 1-(2,6-difluorophenyl) ethan-1-ol were reacted to give title compound as yellow gummy solid (2.47 g, quantitative). LCMS¹: m/z=353 [M+H]⁺.

### Preparation 72: 4-(1-(2, 6-difluorophenyl) ethoxy)-5-(methylcarbamoyl)-1H-pyrrole-2-carboxylic acid

Following the procedure in Example 25/Example 26, Preparation 46, ethyl 4-(1-(2, 6-difluorophenyl) ethoxy)-5-(methylcarbamoyl)-1H-pyrrole-2-carboxylate was reacted to give title compound as a brown solid. (0.90 g, 39%). LCMS¹: m/z=325 [M+H]⁺.

### Preparation 73: 3-(1-(2, 6-difluorophenyl) ethoxy)-N⁵-ethyl-N²-methyl-1H-pyrrole-2, 5-dicarboxamide

Following the procedure in Example 25/Example 26, Preparation 47, 4-(1-(2, 6-difluorophenyl) ethoxy)-5-(methylcarbamoyl)-1H-pyrrole-2-carboxylic acid and ethylamine were reacted to give a white solid as mixture of enantiomers (0.21 g, 76%). The mixture of enantiomers was separated by Chiral Prep-HPLC¹⁷ to afford Enantiomer A (0.058 g, 17%) as a white solid and Enantiomer B (0.080 g, 24%) as a white solid.

Example 55 (Enantiomer A): ¹H NMR: (400 MHz, DMSO-d⁶) δ 11.35 (s, 1H), 8.23 (t, J=4.8 Hz, 1H), 7.45-7.38 (m, 1H), 7.14-7.10 (m, 3H), 6.44 (s, 1H), 5.70 (q, J=6.4 Hz, 1H), 3.19 (m, 2H), 2.83 (d, J=4.8 Hz, 3H), 1.73 (d, J=6.4 Hz, 3H), 1.07 (t, J=7.2 Hz, 3H). LCMS¹: m/z=352 [M+H]⁺. Chiral HPLC¹ t_{R}: 6.06 min, ee %: 93.03%. The absolute configuration has not yet been assigned unambiguously but is believed to be the S enantiomer.

Example 56 (Enantiomer B): ¹H NMR: (400 MHz, DMSO-d⁶) δ 11.35 (s, 1H), 8.23 (t, J=4.8 Hz, 1H), 7.41 (t, J=6.8 Hz, 1H), 7.14-7.10 (m, 3H), 6.44 (s, 1H), 5.71 (q, J=6.4 Hz, 1H), 3.19 (m, 2H), 2.83 (d, J=4.8 Hz, 3H), 1.73 (d, J=6.4 Hz, 3H), 1.07 (t, J=7.2 Hz, 3H). LCMS¹: m/z=352 [M+H]⁺. Chiral HPLC¹ t_{R}: 6.27 min, ee %: 92.75%. The absolute configuration has not yet been assigned unambiguously but is believed to be the R enantiomer.

### Example 57—(S)-3-(1-(2-fluorophenyl) ethoxy)-N²-methyl-N⁵-(2, 2, 2-trifluoroethyl)-1H-pyrrole-2, 5-dicarboxamide

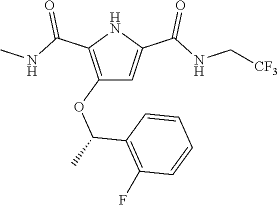 [SMILES: CNC(=O)c1[nH]c(C(=O)NCC(F)(F)F)cc1O[C@@H](C)c1ccccc1F]

### Preparation 74: Ethyl (S)-4-(1-(2-fluorophenyl) ethoxy)-5-(methylcarbamoyl)-1H-pyrrole-2-carboxylate

Following the procedure in Example 25/Example 26, Preparation 45, ethyl 4-hydroxy-5-(methylcarbamoyl)-1H-pyrrole-2-carboxylate and (R)-1-(2-fluorophenyl) ethan-1-ol were reacted to give title compound as a yellow gummy solid (1.2 g, 76%). LCMS¹: m/z=335 [M+H]⁺.

### Preparation 75: (S)-4-(1-(2-fluorophenyl) ethoxy)-5-(methylcarbamoyl)-1H-pyrrole-2-carboxylic acid

Following the procedure in Example 25/Example 26, Preparation 46, ethyl (S)-4-(1-(2-fluorophenyl) ethoxy)-5-(methylcarbamoyl)-1H-pyrrole-2-carboxylate was reacted to give title compound as a pink solid. (0.65 g, 60%). LCMS¹: m/z=307 [M+H]⁺.

### Preparation 76: (S)-3-(1-(2-fluorophenyl) ethoxy)-N2-methyl-N5-(2, 2, 2-trifluoroethyl)-1H-pyrrole-2, 5-dicarboxamide

Following the procedure in Example 105, Preparation 152, (S)-4-(1-(2-fluorophenyl) ethoxy)-5-(methylcarbamoyl)-1H-pyrrole-2-carboxylic acid and 2,2,2-trifluoroethan-1-amine were reacted to give title compound as white solid (0.080 g, 31%). ¹H NMR: (400 MHz, DMSO) δ 11.50 (s, 1H), 8.81 (t, J=6.4 Hz, 1H), 7.56-7.52 (m, 1H), 7.37-7.19 (m, 4H), 6.45 (s, 1H), 5.59 (q, J=6.4 Hz, 1H), 4.08-3.99 (m, 2H), 2.86 (d, J=4.8 Hz, 3H), 1.64 (d, J=6.4 Hz, 3H). LCMS¹: m/z=388 [M+H]⁺. Chiral HPLC³ t_{R}: 4.02 min, ee %: 96.51%.

### Example 58—(S)-3-(1-(2-fluorophenyl) ethoxy)-N⁵-isopropyl-N⁵-methyl-1H-pyrrole-2, 5-dicarboxamide

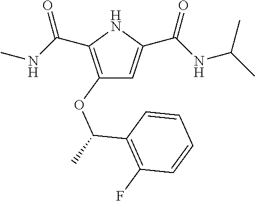 [SMILES: CNC(=O)c1[nH]c(C(=O)NC(C)C)cc1O[C@@H](C)c1ccccc1F]

Following the procedure in Example 105, Preparation 152, (S)-4-(1-(2-fluorophenyl) ethoxy)-5-(methylcarbamoyl)-1H-pyrrole-2-carboxylic acid and isopropyl amine were reacted to give title compound as a white solid (0.10 g, 44%). ¹H NMR: (400 MHz, DMSO) δ 11.40 (s, 1H), 8.10 (d, J=7.2 Hz, 1H), 7.54 (q, J=6.0 Hz, 1H), 7.37-7.31 (m, 1H), 7.26-7.18 (m, 3H), 6.31 (s, 1H), 5.58 (q, J=6.4 Hz, 1H), 3.97-3.89 (m, 1H), 2.85 (d, J=4.4 Hz, 3H), 1.63 (d, J=6.4 Hz, 3H), 1.09 (t, J=6.4 Hz, 6H). LCMS¹: m/z=348 [M+H]⁺. Chiral HPLC² t_{R}: 5.42 min, ee %: 94.81%.

### Example 59—(S)—N⁵-cyclopropyl-3-(1-(2-fluorophenyl) ethoxy)-N²-methyl-1H-pyrrole-2, 5-dicarboxamide

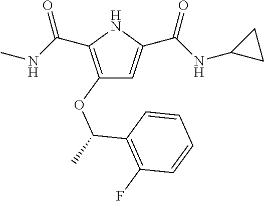 [SMILES: CNC(=O)c1[nH]c(C(=O)NC2CC2)cc1O[C@@H](C)c1ccccc1F]

Following the procedure in Example 105, Preparation 152, (S)-4-(1-(2-fluorophenyl) ethoxy)-5-(methylcarbamoyl)-1H-pyrrole-2-carboxylic acid and cyclopropyl amine were reacted to give title compound as a white solid (0.13 g, 57%). ¹H NMR: (400 MHz, DMSO) δ 11.22 (s, 1H), 8.25 (d, J=4.0 Hz, 1H), 7.54-7.51 (m, 1H), 7.37-7.31 (m, 1H), 7.23-7.18 (m, 3H), 6.33 (s, 1H), 5.57 (q, J=6.4 Hz, 1H), 2.85 (d, J=4.4 Hz, 3H), 2.73-2.67 (m, 1H), 1.63 (d, J=6.4 Hz, 3H), 0.68-0.63 (m, 2H), 0.46 (s, 2H).). LCMS¹: m/z=346 [M+H]⁺. Chiral HPLC¹¹ t_{R}: 13.201 min, ee %: 100%.

### Example 60—(S)-3-(1-(4-chlorophenyl) ethoxy)-N⁵-methyl-N²-(2,2,2-trifluoroethyl)-1H-pyrrole-2,5-dicarboxamide

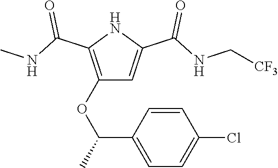 [SMILES: CNC(=O)c1[nH]c(C(=O)NCC(F)(F)F)cc1O[C@@H](C)c1ccc(Cl)cc1]

### Preparation 77: Ethyl (S)-4-(1-(4-chlorophenyl) ethoxy)-5-(methylcarbamoyl)-1H-pyrrole-2-carboxylate

Following the procedure in Example 25/Example 26, Preparation 45, ethyl 4-hydroxy-5-(methylcarbamoyl)-1H-pyrrole-2-carboxylate and (R)-1-(4-chlorophenyl) ethan-1-ol were reacted to give title compound as a yellow gummy solid (1.1 g, 66%) which was used directly in the following step.

### Preparation 78: (S)-4-(1-(4-chlorophenyl) ethoxy)-5-(methylcarbamoyl)-1H-pyrrole-2-carboxylic acid

Following the procedure in Example 25/Example 26, Preparation 46, ethyl (S)-4-(1-(4-chlorophenyl) ethoxy)-5-(methylcarbamoyl)-1H-pyrrole-2-carboxylate was reacted to give title compound as a pink solid (0.58 g, 57%). ¹H NMR (400 MHz, DMSO) δ 12.69 (s, 1H), 11.44 (s, 1H), 7.61-7.57 (t, J=8.0 Hz, 1H), 7.47-7.44 (m, 2H), 7.35-7.30 (m, 2H), 6.34-6.33 (m, 1H), 5.59-5.57 (m, 1H), 2.83-2.82 (d, J=4.0, 3H), 1.62-1.61 (d, J=4.0 Hz, 3H).

### Preparation 79: (S)-3-(1-(4-chlorophenyl) ethoxy)-N2-methyl-N5-(2,2,2-trifluoroethyl)-1H-pyrrole-2,5-dicarboxamide

Following the procedure in Example 105, Preparation 152, (S)-4-(1-(4-chlorophenyl) ethoxy)-5-(methylcarbamoyl)-1H-pyrrole-2-carboxylic acid and 2,2,2-trifluoroethan-1-amine were reacted to give title compound as an off-white solid (0.015 g, 7%). ¹H NMR: (400 MHz, DMSO) δ 11.48 (s, 1H), 8.80 (br s, 1H), 7.49 (d, J=8.0 Hz, 2H), 7.41 (d, J=8.0 Hz, 2H), 7.25 (d, J=4.0 Hz, 1H), 6.45 (s, 1H), 5.41 (d, J=6.4 Hz, 1H), 4.04 (q, J=5.2 Hz, 2H), 2.85 (d, J=4.4 Hz, 3H), 1.59 (d, J=6.0 Hz, 3H). LCMS¹: m/z=402 [M−H]⁻. Chiral HPLC³ t_{R}: 1.47 min, ee %: 100%.

### Example 61—(S)-3-(1-(4-chlorophenyl) ethoxy)-N⁵-isopropyl-N²-methyl-1H-pyrrole-2,5-dicarboxamide

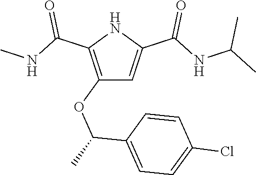 [SMILES: CNC(=O)c1[nH]c(C(=O)NC(C)C)cc1O[C@@H](C)c1ccc(Cl)cc1]

Following the procedure in in Example 105, Preparation 152, (S)-4-(1-(4-chlorophenyl) ethoxy)-5-(methylcarbamoyl)-1H-pyrrole-2-carboxylic acid and isopropylamine were reacted to give title compound as off-white solid (0.098 g, 51%). ¹H NMR: (400 MHz, DMSO) δ 11.33 (s, 1H), 8.06 (d, J=7.2 Hz, 1H), 7.48 (d, J=8.4 Hz, 2H), 7.41 (d, J=8.4 Hz, 2H), 7.20 (d, J=4.8 Hz, 1H), 6.31 (d, J=2.8 Hz, 1H), 5.40 (q, J=6.4 Hz, 1H), 3.97-3.88 (m, 1H), 2.85 (d, J=4.8 Hz, 3H), 1.58 (d, J=6.4 Hz, 3H), 1.09 (d, J=6.4 Hz, 6H). LCMS¹: m/z=362 [M−H]⁻. Chiral HPLC³ t_{R}: 1.97 min. ee %: 95.41%.

### Example 62—(S)-3-(1-(4-chlorophenyl) ethoxy)-N⁵-cyclopropyl-N²-methyl-1H-pyrrole-2,5-dicarboxamide

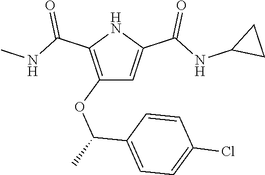 [SMILES: CNC(=O)c1[nH]c(C(=O)NC2CC2)cc1O[C@@H](C)c1ccc(Cl)cc1]

Following the procedure in in Example 105, Preparation 152, (S)-4-(1-(4-chlorophenyl) ethoxy)-5-(methylcarbamoyl)-1H-pyrrole-2-carboxylic acid and cyclopropylamine were reacted to give title compound as off-white solid (0.070 g, 37%). ¹H NMR: (400 MHz, DMSO) δ 11.19 (s, 1H), 8.23 (d, J=3.6 Hz, 1H), 7.48 (d, J=8.4 Hz, 2H), 7.41 (d, J=8.4 Hz, 2H), 7.20 (d, J=4.8 Hz, 1H), 6.32 (d, J=2.4 Hz, 1H), 5.40 (q, J=6.0 Hz, 1H), 2.85 (d, J=4.4 Hz, 3H), 2.59 (br s, 1H), 1.58 (d, J=6.4 Hz, 3H), 0.65 (d, J=1.6 Hz, 2H), 0.44 (d, J=2.0 Hz, 2H). LCMS¹: m/z=360 [M−H]⁻. Chiral HPLC³ t_{R}: 2.96 min, ee %: 95.57%.

### Example 63—Racemic 3-(cyclopropyl(phenyl)methoxy)-N⁵-ethyl-N²-methyl-1H-pyrrole-2,5-dicarboxamide

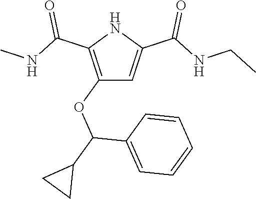 [SMILES: CCNC(=O)c1cc(OC(c2ccccc2)C2CC2)c(C(=O)NC)[nH]1]

### Preparation 80: Ethyl 4-(cyclopropyl(phenyl)methoxy)-5-(methylcarbamoyl)-1H-pyrrole-2-carboxylate

Following the procedure in Example 25/Example 26, Preparation 45, ethyl 4-hydroxy-5-(methylcarbamoyl)-1H-pyrrole-2-carboxylate and cyclopropyl(phenyl)methanol were reacted to give title compound as yellow gummy solid (0.840 g, 36%). LCMS¹: m/z=343 [M+H]⁺.

### Preparation 81: 4-(cyclopropyl(phenyl)methoxy)-5-(methylcarbamoyl)-1H-pyrrole-2-carboxylic acid

Following the procedure in Example 25/Example 26, Preparation 46, ethyl 4-(cyclopropyl(phenyl)methoxy)-5-(methylcarbamoyl)-1H-pyrrole-2-carboxylate was reacted to give title compound as an off-white solid. (0.65 g, 84%). LCMS¹: m/z=315 [M+H]⁺.

### Preparation 82: 3-(cyclopropyl(phenyl)methoxy)-N5-ethyl-N2-methyl-1H-pyrrole-2,5-dicarboxamide

Following the procedure in Example 25/Example 26, Preparation 47, 4-(cyclopropyl(phenyl)methoxy)-5-(methylcarbamoyl)-1H-pyrrole-2-carboxylic acid and ethylamine were reacted to give title compound as white solid. (0.027 g, 6%). ¹H NMR: (400 MHz, DMSO) δ 11.20 (s, 1H), 8.17 (t, J=4.8 Hz, 1H), 7.46 (d, J=7.2 Hz, 2H), 7.35 (t, J=7.2 Hz, 2H), 7.28-7.25 (m, 2H), 6.26 (s, 1H), 4.58 (d, J=8.8 Hz, 1H), 3.33-3.31 (m, 2H), 2.88 (d, J=4.8 Hz, 3H), 1.48-1.43 (m, 1H), 1.05 (t, J=7.2 Hz, 3H), 0.66-0.42 (m, 4H). LCMS¹: m/z=342 [M+H]⁺.

### Example 64—3-(1-(2,6-dimethylphenyl) ethoxy)-N⁵-ethyl-N²-methyl-1H-pyrrole-2,5-dicarboxamide, Enantiomer A

### Example 65—3-(1-(2,6-dimethylphenyl) ethoxy)-N⁵-ethyl-N²-methyl-1H-pyrrole-2,5-dicarboxamide, Enantiomer B

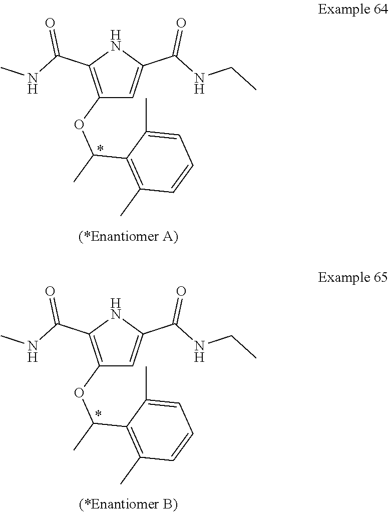

### Preparation 83: 1-(2,6-dimethylphenyl) ethan-1-ol

1-(2,6-dimethylphenyl) ethan-1-one (5 g, 33.78 mmol) was dissolved in THE (100 mL) at room temperature under a nitrogen atmosphere. Lithium aluminium hydride (33.78 mL, 67.56 mmol) was added to the reaction mixture at 0° C. The suspension was allowed to stir at room temperature for 16 h. To the reaction mixture was slowly added water (300 mL) and extracted with EtOAc (300 mL). The organic layer was washed with brine (300 mL) and dried over anhydrous Na₂SO₄ and evaporated. The resulting residue was purified by normal phase chromatography, eluting with (30:70) ethyl acetate/hexane to yield 1-(2,6-dimethylphenyl) ethan-1-ol (4.3 g, 50%) as a white solid. ¹H NMR: (400 MHz, DMSO-d⁶) δ 6.95 (s, 1H), 6.92 (d, J=6.4 Hz, 2H), 5.03 (m, 1H), 4.98 (d, J=3.2 Hz, 1H), 2.37 (s, 6H), 1.34 (d, J=6.8 Hz, 3H).

### Preparation 84: Ethyl 4-(1-(2,6-dimethylphenyl) ethoxy)-5-(methylcarbamoyl)-1H-pyrrole-2-carboxylate

Following the procedure in Example 25/Example 26, Preparation 45, ethyl 4-hydroxy-5-(methylcarbamoyl)-1H-pyrrole-2-carboxylate and 1-(2,6-dimethylphenyl) ethan-1-ol were reacted to give title compound as a yellow gummy solid (1.0 g, 47%). LCMS¹: m/z=345 [M+H]⁺.

### Preparation 85: 4-(1-(2,6-dimethylphenyl) ethoxy)-5-(methylcarbamoyl)-1H-pyrrole-2-carboxylic acid

Following the procedure in Example 25/Example 26, Preparation 46, ethyl 4-(1-(2,6-dimethylphenyl) ethoxy)-5-(methylcarbamoyl)-1H-pyrrole-2-carboxylate was reacted to give title compound as a pink solid (0.5 g, 54%). LCMS¹: m/z=315 [M−H]⁻.

### Preparation 86: 3-(1-(2,6-dimethylphenyl) ethoxy)-N5-ethyl-N2-methyl-1H-pyrrole-2,5-dicarboxamide

Following the procedure in Example 25/Example 26, Preparation 47, 4-(1-(2,6-dimethylphenyl) ethoxy)-5-(methylcarbamoyl)-1H-pyrrole-2-carboxylic acid and ethylamine were reacted to give an off-white solid (0.23 g, 44%) as mixture of enantiomers. The mixture of enantiomer is separated by Chiral Prep-HPLC¹⁰ to afford Enantiomer A (0.049 g, 10%) as an off-white solid and Enantiomer B (0.049 g, 10%) as an off-white solid.

Example 64 (Enantiomer A): ¹H NMR: (400 MHz, DMSO) δ 11.27 (s, 1H), 8.22 (t, J=5.2 Hz, 1H), 7.14 (d, J=4.8 Hz, 1H), 7.07-7.03 (m, 1H), 6.98 (d, J=7.2 Hz, 2H), 5.94 (s, 1H), 5.58-5.57 (m, 1H), 3.16 (m, 2H), 2.85 (d, J=4.4 Hz, 3H), 2.38 (s, 6H), 1.65 (d, J=6.4 Hz, 3H), 1.05 (t, J=7.6 Hz, 3H). LCMS¹: m/z=342 [M−H]⁻. Chiral HPLC⁶ t_{R}: 4.76 min, ee %: 100%. The absolute configuration has not yet been assigned unambiguously but is believed to be the S enantiomer.

Example 65 (Enantiomer B): ¹H NMR: (400 MHz, DMSO-d⁶) δ 11.27 (s, 1H), 8.22 (t, J=4.8 Hz, 1H), 7.14 (d, J=4.8 Hz, 1H), 7.07-6.98 (m, 3H), 5.94 (s, 1H), 5.57 (m, 1H), 3.16 (m, 2H) 2.85 (d, J=4.4 Hz, 3H), 2.38 (s, 6H), 1.65 (d, J=6.8 Hz, 3H), 1.05 (t, J=7.2 Hz, 3H). LCMS¹: m/z=342 [M−H]⁻. Chiral HPLC⁶ t_{R}: 5.02 min, ee %: 100%. The absolute configuration has not yet been assigned unambiguously but is believed to be the R enantiomer.

### Example 66—3-(1-(2, 6-dichlorophenyl) ethoxy)-N⁵-ethyl-N²-methyl-1H-pyrrole-2, 5-dicarboxamide, Enantiomer A

### Example 67—3-(1-(2, 6-dichlorophenyl) ethoxy)-N⁵-ethyl-N²-methyl-1H-pyrrole-2, 5-dicarboxamide, Enantiomer B

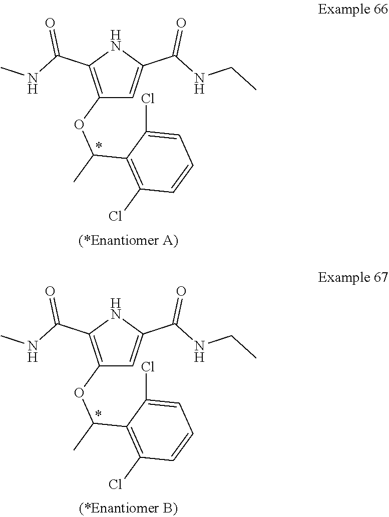

### Preparation 87: Ethyl 4-(1-(2, 6-dichlorophenyl) ethoxy)-5-(methylcarbamoyl)-1H-pyrrole-2-carboxylate

Following the procedure in Example 25/Example 26, Preparation 45, ethyl 4-hydroxy-5-(methylcarbamoyl)-1H-pyrrole-2-carboxylate and 1-(2,6-dichlorophenyl) ethan-1-ol were reacted to give title compound as a yellow gummy solid (2 g, 84%). LCMS¹: m/z=385 [M+H]⁺.

### Preparation 88: 4-(1-(2, 6-dichlorophenyl) ethoxy)-5-(methylcarbamoyl)-1H-pyrrole-2-carboxylic acid

Following the procedure in Example 25/Example 26, Preparation 46, ethyl 4-(1-(2,6-dichlorophenyl)ethoxy)-5-(methylcarbamoyl)-1H-pyrrole-2-carboxylate was reacted to give title compound as brown solid. (0.360 g, 19%). LCMS¹: m/z=357 [M+H]⁺.

### Preparation 89: 3-(1-(2, 6-dichlorophenyl) ethoxy)-N⁵-ethyl-N²-methyl-1H-pyrrole-2, 5-dicarboxamide

Following the procedure in Example 25/Example 26, Preparation 47, 4-(1-(2, 6-dichlorophenyl) ethoxy)-5-(methylcarbamoyl)-1H-pyrrole-2-carboxylic acid and ethylamine were reacted to give white solid as mixture of enantiomers (0.21 g, 76%). The mixture of enantiomers was separated by Chiral Prep-HPLC¹¹ to afford Enantiomer A (0.058 g, 17%) as a white solid Enantiomer B (0.065 g, 20%) as a white solid.

Example 66 (Enantiomer A): ¹H NMR: (400 MHz, DMSO-d⁶) δ 11.43 (s, 1H), 8.24 (t, J=5.2 Hz, 1H), 7.49 (d, J=8.4 Hz, 2H), 7.35 (t, J=8 Hz, 1H), 7.19 (d, J=4.8 Hz, 1H), 6.24 (s, 1H), 5.96 (q, J=6.8 Hz, 1H), 3.20-3.12 (m, 2H), 2.85 (d, J=4.8 Hz, 3H), 1.75 (d, J=6.8 Hz, 3H), 1.06 (t, J=7.2 Hz, 3H). LCMS¹: m/z=384 [M+H]⁺. Chiral HPLC¹ t_{R}: 3.17 min, ee %: 100%

Example 67 (Enantiomer B): ¹H NMR: (400 MHz, DMSO-d⁶) δ 11.42 (s, 1H), 8.24 (t, J=4.8 Hz, 1H), 7.49 (d, J=8 Hz, 2H), 7.36 (t, J=8 Hz, 1H), 7.19 (d, J=4.8 Hz, 1H), 6.24 (s, 1H), 5.97 (q, J=6.4 Hz, 1H), 3.18 (m, 2H), 2.85 (d, J=4.8 Hz, 3H), 1.76 (d, J=6.4 Hz, 3H), 1.06 (t, J=7.2 Hz, 3H). LCMS¹: m/z=384 [M+H]⁺. Chiral HPLC¹ t_{R}: 5.15 min, ee %: 99.50%.

### Example 68—(S)-3-(1-(2-fluorophenyl) ethoxy)-N-methyl-5-(5-methyl-1H-imidazol-2-yl)-1H-pyrrole-2-carboxamide

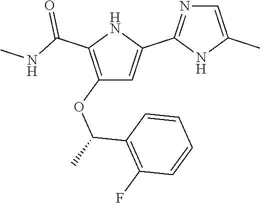

### Preparation 90: (S)-3-(1-(2-fluorophenyl) ethoxy)-5-(hydroxymethyl)-N-methyl-1H-pyrrole-2-carboxamide

Lithium aluminium hydride (1.7 g, 44.91 mmol) in THE (50 mL) was cooled at 0° C. under a nitrogen atmosphere. Ethyl (S)-4-(1-(2-fluorophenyl) ethoxy)-5-(methylcarbamoyl)-1H-pyrrole-2-carboxylate (5 g, 14.97 mmol) was dropwise added to reaction mixture under nitrogen. The resulting mixture was stirred at room temperature for 16 h. The reaction mixture was quenched with chilled water (300 mL) and extracted with ethyl acetate (300 mL). The organic layer was washed with brine solution (2×300 mL), dried over anhydrous sodium sulphate and concentrated under vacuum to afford crude material. The crude material was purified by normal phase chromatography column chromatography, eluting with 70% ethyl acetate in hexane to afford title product as a yellow solid (2.6 g, 59%). LCMS¹: m/z=293 [M+H]⁺.

### Preparation 91: (S)-3-(1-(2-fluorophenyl) ethoxy)-5-formyl-N-methyl-1H-pyrrole-2-carboxamide

(S)-3-(1-(2-fluorophenyl) ethoxy)-5-(hydroxymethyl)-N-methyl-1H-pyrrole-2-carboxamide (2.5 g, 8.56 mmol) was dissolved in DCM (25 mL) under a nitrogen at room temperature. Dess-Martin periodinane (7.26 g, 17.12 mmol) was added portion wise to the reaction mixture at 0° C. The resulting mixture was stirred at room temperature for 3 h. The reaction mixture was quenched with saturated sodium bicarbonate solution (300 mL), the biphasic mixture was stirred for 20 min then extracted with DCM (2×300 mL). The organic layer was washed with brine solution (2×150 mL), dried over anhydrous sodium sulphate and concentrated under vacuum to afford crude material (1.9 g, 76%). LCMS¹: m/z=291 [M+H]⁺.

### Preparation 92: (S)-3-(1-(2-fluorophenyl) ethoxy)-N-methyl-5-(5-methyl-1H-imidazol-2-yl)-1H-pyrrole-2-carboxamide

(S)-3-(1-(2-fluorophenyl) ethoxy)-5-formyl-N-methyl-1H-pyrrole-2-carboxamide (0.90 g, 3.10 mmol) was dissolved in methanol (18 mL) under nitrogen at room temperature. Ammonium hydroxide solution in water (2.25 mL, 2.5V) was added drop wise to the reaction mixture at room temperature. The resulting mixture was stirred at room temperature for 20 min. Pyruvaldehyde (1.56 g, 21.72 mmol) was added drop wise to reaction mixture at 0° C. The resulting mixture was stirred at 50° C. for 5 h. The reaction mixture was concentrated under vacuum to afford crude material. The crude material was purified flash chromatography in reverse phase using Biotage select with UV detector, silica: C18 silica 50 μm and product was eluting with (56:44) acetonitrile/water). Fraction was combined and lyophilized to give title product as a yellow solid (0.45 g, 42%). ¹H NMR: (400 MHz, DMSO) δ 13.98 (br s, 1H), 11.82 (s, 1H), 7.54 (t, J=7.6 Hz, 1H), 7.38-7.30 (m, 2H), 7.26-7.19 (m, 3H), 6.61 (d, J=2.4 Hz, 1H), 5.58 (q, J=6.4 Hz, 1H), 2.89 (d, J=4.8 Hz, 3H), 2.27 (s, 3H), 1.69 (d, J=6.4 Hz, 3H). LCMS¹: m/z=343 [M+H]⁺. Chiral HPLC² t_{R}: 5.41 min, ee % 89.37%,

### Example 69—(S)-3-(1-(4-chlorophenyl) ethoxy)-N-methyl-5-(5-methyl-1H-imidazol-2-yl)-1H-pyrrole-2-carboxamide

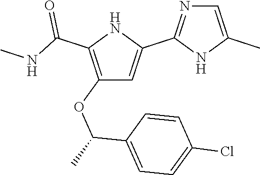

### Preparation 93: (S)-3-(1-(4-chlorophenyl) ethoxy)-5-(hydroxymethyl)-N-methyl-1H-pyrrole-2-carboxamide

Following the procedure in Example 68, Preparation 90, ethyl (S)-4-(1-(4-chlorophenyl) ethoxy)-5-(methylcarbamoyl)-1H-pyrrole-2-carboxylate was reacted to give title compound as a yellow solid (1.0 g, 22%). LCMS¹: m/z=309 [M+H]⁺.

### Preparation 94: (S)-3-(1-(4-chlorophenyl) ethoxy)-5-formyl-N-methyl-1H-pyrrole-2-carboxamide

Following the procedure in Example 68, Preparation 91, (S)-3-(1-(4-chlorophenyl) ethoxy)-5-(hydroxymethyl)-N-methyl-1H-pyrrole-2-carboxamide was reacted to give title compound as a yellow liquid. (1.9 g, 76%) which was used directly in the next step.

### Preparation 95: (S)-3-(1-(4-chlorophenyl) ethoxy)-N-methyl-5-(5-methyl-1H-imidazol-2-yl)-1H-pyrrole-2-carboxamide

Following the procedure in Example 68, Preparation 92, (S)-3-(1-(4-chlorophenyl) ethoxy)-5-formyl-N-methyl-1H-pyrrole-2-carboxamide and pyruvaldehyde were reacted to give title compound as an orange solid (0.13 g, 27%). ¹H NMR: (400 MHz, DMSO) δ 13.98 (br s, 1H), 11.82 (s, 1H), 7.47 (d, J=6.8 Hz, 1H), 10.90 (s, 1H), 7.42 (d, J=8.4 Hz, 2H), 7.07-6.56 (m, 2H), 6.17-6.12 (m, 1H), 5.44-5.41 (m, 1H), 2.86 (d, J=4.8 Hz, 3H), 2.07 (s, 3H), 1.60 (d, J=6.4 Hz, 3H). LCMS¹: m/z=359 [M+H]⁺. Chiral HPLC² t_{R}: 6.25 min, ee %: 96.87%.

### Example 70—N⁵-ethyl-3-(1-(4-ethynylphenyl) ethoxy)-N²-methyl-1H-pyrrole-2,5-dicarboxamide, Enantiomer A

### Example 71—N⁵-ethyl-3-(1-(4-ethynylphenyl) ethoxy)-N²-methyl-1H-pyrrole-2,5-dicarboxamide, Enantiomer B

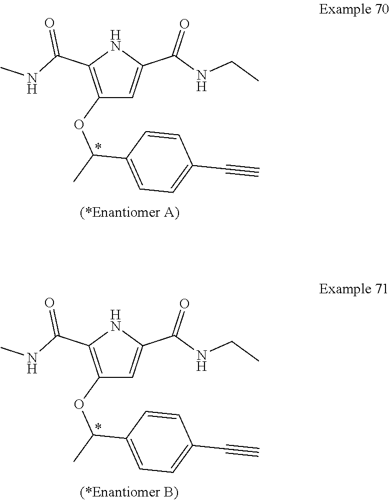

### Preparation 96: 1-(4-iodophenyl) ethan-1-ol

Following the procedure in Example 10, Preparation 19, 1-(4-iodophenyl) ethan-1-one was reacted to give title compound as a yellow oil (9.2 g, 89%). ¹H NMR: (400 MHz, DMSO-d⁶) δ 7.66 (d, J=8.4 Hz, 2H), 7.15 (d, J=8.4 Hz, 2H), 5.23 (d, J=4.4 Hz, 1H), 4.67 (m, 1H), 1.28 (d, J=6.4 Hz, 3H).

### Preparation 97: 1-(4-((tri-isopropyl silyl) ethynyl) phenyl) ethan-1-ol

1-(4-iodophenyl) ethan-1-ol (5.0 g, 20.16 mmol), PdCl₂(dppf) (0.28 g, 0.40 mmol) and copper iodide (0.07 g, 0.40 mmol) were dissolved in piperidine (50 mL) at room temperature. The mixture was degassed using nitrogen for 10 min. Triethylamine (50 mL, 10 mmol) and (triisopropylsilyl)acetylene (4.5 mL, 20.16 mmol) were added to the reaction mixture and degassed using nitrogen. The resulting mixture was allowed to stir at 65° C. for 12 h. The resulting solution was evaporated under the vacuum and diluted with water (500 mL) and ethyl acetate (500 mL). The combined organics were dried over Na2SO4, filtered and evaporated. The residue was purified by normal phase chromatography, eluting with (10:90) ethyl acetate/hexane to yield 1-(4-((tri-isopropyl silyl) ethynyl) phenyl) ethan-1-ol (8 g, 85%) as a brown oil. ¹H NMR: (400 MHz, DMSO) δ 7.48 (d, J=8.4 Hz, 2H), 7.33 (d, J=8.4 Hz, 2H), 4.91 (q, J=6.8 Hz, 1H), 1.49 (d, J=6.4 Hz, 3H), 1.14 (s, 18H).

### Preparation 98: 1-(4-ethynylphenyl) ethan-1-ol

To a stirred solution of 1-(4-((tri-isopropyl silyl) ethynyl) phenyl) ethan-1-ol (5.3 g, 17.54 mmol) in tetrahydrofuran (53 mL) under nitrogen was added tetra-n-butyl ammonium fluoride (35 mL, 35 mmol) at 0° C. The reaction mixture was allowed to stir at room temperature for 2 h. The resulting solution was diluted with cold water (500 mL) and ethyl acetate (500 mL). The combined organics were dried over Na₂SO₄, filtered and evaporated. The residue was purified by normal phase chromatography, eluting with (10:90) ethyl acetate/hexane to yield 1-(4-ethynylphenyl) ethan-1-ol (2 g, 79%) as a yellow oil. ¹H NMR: (400 MHz, DMSO-d⁶) δ 7.49 (d, J=8.0 Hz, 2H), 7.35 (d, J=8 Hz, 2H), 4.94-4.89 (m, 1H), 3.08 (s, 1H), 1.50 (d, J=6.4 Hz, 3H).

### Preparation 99: Ethyl 4-(1-(4-ethynylphenyl) ethoxy)-5-(methyl carbamoyl)-1H-pyrrole-2-carboxylate

Following the procedure in Example 25/Example 26, Preparation 45, ethyl 4-hydroxy-5-(methyl carbamoyl)-1H-pyrrole-2-carboxylate and 1-(4-ethynylphenyl) ethan-1-ol were reacted to give title product as a light yellow oil (1.0 g, 41%). LCMS¹: m/z=341 [M+H]⁺.

### Preparation 100: 4-(1-(4-ethynylphenyl) ethoxy)-5-(methyl carbamoyl)-1H-pyrrole-2-carboxylic acid

Following the procedure in Example 25/Example 26, Preparation 46, ethyl 4-(1-(4-ethynylphenyl) ethoxy)-5-(methyl carbamoyl)-1H-pyrrole-2-carboxylate was reacted to give title compound as an off-white solid (0.45 g, 52%). ¹H NMR: (400 MHz, DMSO-d⁶) δ 12.64 (br s, 1H), 11.31 (s, 1H), 7.49-7.43 (m, 4H), 7.33 (d, J=4.4 Hz, 1H), 6.33 (d, J=2.4 Hz, 1H), 5.43 (q, J=6.4 Hz, 1H), 4.17 (s, 1H), 4.03 (q, J=7.2 Hz, 1H), 2.83 (d, J=4.8 Hz, 3H), 1.58 (d, J=6.4 Hz, 3H).

### Preparation 101: N⁵-ethyl-3-(1-(4-ethynylphenyl) ethoxy)-N²-methyl-1H-pyrrole-2,5-dicarboxamide

Following the procedure in Example 25/Example 26, Preparation 47, 4-(1-(4-ethynylphenyl) ethoxy)-5-(methyl carbamoyl)-1H-pyrrole-2-carboxylic acid and ethylamine were reacted to give a white solid as mixture of enantiomers (0.14 g, 46%). The mixture of enantiomers was separated by Chiral Prep-HPLC¹² to afford Enantiomer A (0.027 g, 18%) as a white solid and Enantiomer B (0.044 g, 29%) as a light orange solid.

Example 70 (Enantiomer A): ¹H NMR: (400 MHz, DMSO-d⁶) δ 11.24 (s, 1H), 8.20 (t, J=4.8 Hz, 1H), 7.46 (s, 4H), 7.21 (d, J=4.4 Hz, 1H), 6.30 (s, 1H), 5.40 (d, J=6.4 Hz, 1H), 4.18 (s, 1H), 3.17 (m, 2H), 2.85 (d, J=4.8 Hz, 3H), 1.58 (d, J=6.4 Hz, 3H), 1.05 (t, J=7.2 Hz, 3H). LCMS¹: m/z=338 [M−H]⁻. Chiral HPLC¹¹ t_{R}: 10.31 min, ee %: 100%.

Example 71 (Enantiomer B): ¹H NMR: (400 MHz, DMSO-d⁶) δ 11.24 (s, 1H), 8.20 (t, J=4.8 Hz, 1H), 7.46 (s, 4H), 7.21 (d, J=4.4 Hz, 1H), 6.30 (d, J=2 Hz, 1H), 5.40 (q, J=6.4 Hz, 1H), 4.18 (s, 1H), 3.17 (m, 2H), 2.85 (d, J=4.4 Hz, 3H), 1.58 (d, J=6.4 Hz, 3H), 1.05 (t, J=7.2 Hz, 3H). LCMS¹: m/z=338 [M−H]⁻. Chiral HPLC¹¹ t_{R}: 11.22 min, ee % 94.55%.

### Example 72—(S)-3-bromo-N²-ethyl-4-(1-(2-fluorophenyl)ethoxy)-N⁵-methyl-1H-pyrrole-2,5-dicarboxamide

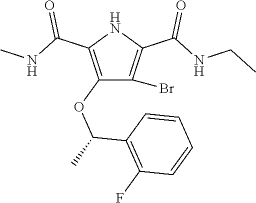 [SMILES: CCNC(=O)c1[nH]c(C(=O)NC)c(O[C@@H](C)c2ccccc2F)c1Br]

### Preparation 102: (S)—N5-ethyl-3-(1-(2-fluorophenyl) ethoxy)-N2-methyl-1H-pyrrole-2, 5-dicarboxamide

Following the procedure in Example 105, Preparation 152, (S)-4-(1-(2-fluorophenyl) ethoxy)-5-(methylcarbamoyl)-1H-pyrrole-2-carboxylic acid and ethylamine were reacted to give title compound as a white solid (0.20 g, 61%). LCMS¹: m/z=334 [M+H]⁺.

### Preparation 103: (S)-3-bromo-N²-ethyl-4-(1-(2-fluorophenyl) ethoxy)-N-methyl-1H-pyrrole-2,5-dicarboxamide

(S)—N5-ethyl-3-(1-(2-fluorophenyl) ethoxy)-N2-methyl-1H-pyrrole-2,5-dicarboxamide (0.17 g, 0.52 mmol) was dissolved in DMF (1.7 mL) under nitrogen at room temperature. N-bromosuccinimide (0.094 g, 0.52 mmol) was added portion wise to the reaction mixture at 0° C. The resulting mixture was stirred at room temperature for 16 h. The reaction mixture was quenched with water (30 mL) and extracted with ethyl acetate (2×30 mL). The organic layer was washed with brine solution (2×30 mL) and concentrated under vacuum to afford crude material. The crude material was purified flash chromatography in reverse phase using Biotage select with UV detector, silica: C18 silica 50 μm and product was eluting with (60:40) acetonitrile/water). Fraction was combined and lyophilized to give title product as a white solid (0.20 g, 91%). ¹H NMR: (400 MHz, DMSO) δ 11.83 (s, 1H), 8.05 (t, J=4.8 Hz, 1H), 7.67 (t, J=6.4 Hz, 1H), 7.39-7.13 (m, 4H), 5.68 (q, J=6.4 Hz, 1H), 3.25-3.19 (m, 2H), 2.69 (d, J=4.8 Hz, 3H), 1.64 (d, J=6.4 Hz, 3H), 1.10 (t, J=7.2 Hz, 3H). LCMS¹: m/z=412 [M+H]⁺.

### Example 73—Racemic N⁵-ethyl-3-(2-hydroxy-1-phenylethoxy)-N²-methyl-1H-pyrrole-2,5-dicarboxamide

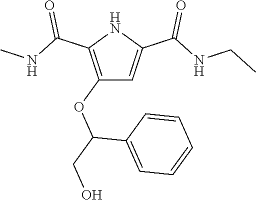 [SMILES: CCNC(=O)c1cc(OC(CO)c2ccccc2)c(C(=O)NC)[nH]1]

### Preparation 104: 2-hydroxy-2-phenylethyl 4-methylbenzenesulfonate

1-phenylethane-1, 2-diol (5 g, 36.21 mmol) was dissolved in DCM (40 mL) at room temperature under nitrogen. Triethylamine (7.32 g, 72.42 mmol) was added to the reaction mixture followed by addition of 4-toluenesulfonyl chloride (8.90 g, 46.71 mmol). The reaction mixture was allowed to stir at room temperature for 3 h. The reaction mixture was slowly added to water (300 mL) and extracted with DCM (2×300 mL). The organic layer was washed with brine (2×300 mL) and dried over anhydrous sodium sulphate and concentrated under vacuum to afford crude material. The crude material was purified by normal phase chromatography column chromatography, eluting with (8:92) ethyl acetate/hexane. Solvent reduction afforded product as a light yellow liquid (6.5 g, 61%). ¹H NMR: (400 MHz, DMSO) δ 7.76-7.69 (m, 2H), 7.43 (d, J=8.0 Hz, 2H), 7.37-7.25 (m, 5H), 5.78-5.69 (m, 1H), 4.76 (t, J=6.0 Hz, 1H), 4.06-3.95 (m, 2H), 2.41 (s, 3H).

### Preparation 105: Ethyl 5-(methylcarbamoyl)-4-(1-phenyl-2-(tosyloxy) ethoxy)-1H-pyrrole-2-carboxylate

Following the procedure in Example 27/Example 28, Preparation 48, ethyl 4-hydroxy-5-(methylcarbamoyl)-1H-pyrrole-2-carboxylate and 2-hydroxy-2-phenylethyl 4-methylbenzenesulfonate were reacted to give title compound as a yellow gummy solid (2 g, 48%). LCMS¹: m/z=487 [M+H]⁺.

### Preparation 106: 5-(methylcarbamoyl)-4-(1-phenyl-2-(tosyloxy) ethoxy)-1H-pyrrole-2-carboxylic acid

Following the procedure in Example 25/Example 26, Preparation 46, ethyl 5-(methylcarbamoyl)-4-(1-phenyl-2-(tosyloxy) ethoxy)-1H-pyrrole-2-carboxylate was reacted to give title compound as a brown gummy solid (0.65 g, crude). LCMS¹: m/z=459 [M+H]⁺.

### Preparation 107: 2-((5-(ethyl carbamoyl)-2-(methylcarbamoyl)-1H-pyrrol-3-yl) oxy)-2-phenylethyl 4-methylbenzenesulfonate

Following the procedure in Example 25/Example 26, Preparation 47, 5-(methylcarbamoyl)-4-(1-phenyl-2-(tosyloxy) ethoxy)-1H-pyrrole-2-carboxylic acid and ethylamine were reacted to give title compound as an orange liquid. (0.55 g, crude). LCMS¹: m/z=486 [M+H]⁺.

### Preparation 108: N-ethyl-3-(2-hydroxy-1-phenylethoxy)-NV-methyl-1H-pyrrole-2, 5-dicarboxamide

2-((5-(ethyl carbamoyl)-2-(methylcarbamoyl)-1H-pyrrol-3-yl) oxy)-2-phenylethyl 4-methylbenzene sulfonate (0.50 g, 0.30 mmol) was dissolved in ethanol (7.5 mL) and water (3 mL) at room temperature. Sodium hydroxide (0.086 g, 2.16 mmol) was added to the reaction mixture at room temperature and allowed to stir at 60° C. for 16 h. The resulting solution was cooled to room temperature and concentrated under vacuum distillation. The resulting residue was diluted with water (40 mL) and extracted with ethyl acetate (2×40 mL). Fractions were combined, dried over anhydrous sodium sulphate and concentrated under vacuum to afford crude. The crude material was purified by reverse phase column chromatography using Biotage select; Silica C18; the product was eluted 40:60 (Acetonitrile:water). Fractions were lyophilized to give title product as a white solid (0.004 g, 5%). ¹H NMR: (400 MHz, DMSO) δ 11.23 (s, 1H), 8.20 (t, J=5.2 Hz, 1H), 7.61 (d, J=4.8 Hz, 1H), 7.41-7.28 (m, 5H), 6.09 (s, 1H), 5.45 (t, J=5.6 Hz, 1H), 5.09 (t, J=2.8 Hz, 1H), 3.75-3.70 (m, 1H), 3.64-3.61 (m, 1H), 3.18-3.12 (m, 2H), 2.86 (d, J=4.4 Hz, 3H), 1.04 (t, J=7.2 Hz, 3H). LCMS¹: m/z=332 [M+H]⁺.

### Example 74—(S)—N²-ethyl-4-(1-(2-fluorophenyl) ethyl)-N⁵-dimethyl-1H-pyrrole-2,5-dicarboxamide

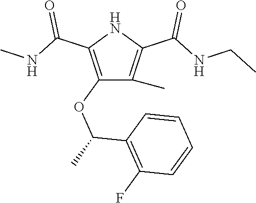 [SMILES: CCNC(=O)c1[nH]c(C(=O)NC)c(O[C@@H](C)c2ccccc2F)c1C]

Example 72 (0.17 g, 0.41 mmol) was dissolved in 1,4-dioxane (1.5 mL) under argon. 2,4,6-trimethyl-1,3,5,2,4,6-trioxatriborinane (0.15 g, 1.24 mmol) was added to the reaction mixture followed by potassium carbonate (0.15 g, 1.15 mmol) and water (0.2 mL) at room temperature. The suspension was degassed with argon for 15 min. Tetrakis(triphenylphosphine)palladium(0) (0.047 g, 0.04 mmol) was added to the reaction mixture. The reaction mixture was stirred at rt for 6 h then heated at 110° C. for 12 h. The reaction mixture was diluted with ethyl acetate (30 mL) and filtered through a celite pad. The organic phase was dried over anhydrous sodium sulphate, filtered and concentrated under reduced pressure to afford crude material. The residue was purified by reverse phase column chromatography using Biotage select; Silica C18; the product was eluted 40:60 (Acetonitrile:water). Fractions were lyophilized to give title product as an off-white solid (0.005 g, 3%). ¹H NMR: (400 MHz, DMSO) δ 11.18 (s, 1H), 8.05 (t, J=6.8 Hz, 1H), 7.66-7.62 (m, 1H), 7.40-7.34 (m, 1H), 7.25-7.15 (m, 3H), 5.33 (q, J=6.4 Hz, 1H), 3.23-3.16 (m, 2H), 2.72 (d, J=4.8 Hz, 3H), 2.14 (s, 3H), 1.62 (d, J=6.8 Hz, 3H), 1.09 (t, J=7.2 Hz, 3H). LCMS¹: m/z=346 [M−H]⁻.

### Example 75—N⁵-ethyl-3-(1-(4-methoxy phenyl) ethoxy)-N²-methyl-1H-pyrrole-2,5-dicarboxamide, Enantiomer A

### Example 76—N⁵-ethyl-3-(1-(4-methoxy phenyl) ethoxy)-N²-methyl-1H-pyrrole-2,5-dicarboxamide, Enantiomer B

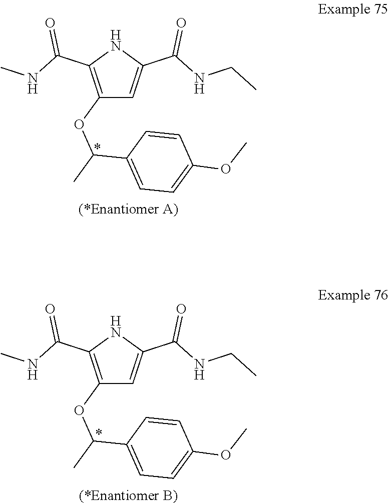

### Preparation 109: Ethyl 4-(1-(4-methoxy phenyl) ethoxy)-5-(methylcarbamoyl)-1H-pyrrole-2-carboxylate

Following the procedure in Example 27/Example 28, Preparation 48, ethyl 4-hydroxy-5-(methylcarbamoyl)-1H-pyrrole-2-carboxylate and 1-(4-methoxy phenyl) ethan-1-ol were reacted to give a yellow gummy solid (1.19 g, 33%). LCMS¹: m/z=347 [M+H]⁺.

### Preparation 110: 4-(1-(4-methoxy phenyl) ethoxy)-5-(methylcarbamoyl)-1H-pyrrole-2-carboxylic acid

Following the procedure in Example 25/Example 26, Preparation 46, ethyl 4-(1-(4-methoxy phenyl) ethoxy)-5-(methylcarbamoyl)-1H-pyrrole-2-carboxylate was reacted to give an off-white solid. (0.39 g, 35%). LCMS¹: m/z=319 [M+H]⁺.

### Preparation 111: N⁵-ethyl-3-(1-(4-methoxy phenyl) ethoxy)-N²-methyl-1H-pyrrole-2,5-dicarboxamide

Following the procedure in Example 25/Example 26, Preparation 47, 4-(1-(5-chloropyridin-2-yl) ethoxy)-5-(methylcarbamoyl)-1H-pyrrole-2-carboxylic acid and ethylamine were reacted to give mixture of enantiomers as an off-white solid (0.14 g, 76%). The mixture of enantiomers was separated by Chiral Prep-HPLC² to afford Enantiomer A (0.043 g, 10%) as an off-white solid and Enantiomer B (0.033 g, 8%) as an off-white solid.

Example 75 (Enantiomer A): ¹H NMR: (400 MHz, DMSO) δ 11.19 (s, 1H), 8.20 (t, J=5.2 Hz, 1H), 7.36 (d, J=8.8 Hz, 2H), 7.18 (q, J=4.4, Hz, 1H), 6.90 (d, J=8.4 Hz, 2H), 6.35 (s, 1H), 5.31 (q, J=6.4 Hz, 1H), 3.72 (s, 3H), 3.35-3.21 (m, 2H), 2.85 (d, J=4.8 Hz, 3H), 1.58 (d, J=6.4 Hz, 3H), 1.06 (t, J=7.2 Hz, 3H). LCMS¹: m/z=344 [M−H]⁻. Chiral HPLC³ t_{R}: 2.87 min, ee %: 99.31%. The absolute configuration has not yet been assigned unambiguously but is believed to be the S enantiomer.

Example 76 (Enantiomer B): ¹H NMR: (400 MHz, DMSO) δ 11.19 (s, 1H), 8.20 (t, J=5.2 Hz, 1H), 7.36 (d, J=8.8 Hz, 2H), 7.18 (q, J=4.4, Hz, 1H), 6.90 (d, J=8.4 Hz, 2H), 6.35 (s, 1H), 5.31 (q, J=6.4 Hz, 1H), 3.72 (s, 3H), 3.35-3.21 (m, 2H), 2.85 (d, J=4.8 Hz, 3H), 1.58 (d, J=6.4 Hz, 3H), 1.06 (t, J=7.2 Hz, 3H). LCMS¹: m/z=346 [M+H]⁺. Chiral HPLC³ t_{R}: 3.37 min, ee %: 97.65%. The absolute configuration has not yet been assigned unambiguously but is believed to be the R enantiomer.

### Example 77—Racemic N⁵-ethyl-N²-methyl-3-(2-methyl-1-phenylpropoxy)-1H-pyrrole-2,5-dicarboxamide

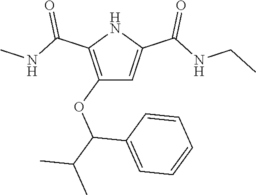 [SMILES: CCNC(=O)c1cc(OC(c2ccccc2)C(C)C)c(C(=O)NC)[nH]1]

### Preparation 112: (1-bromo-2-methylpropyl) benzene

Following the procedure in Example 1, Preparation 1, 2-methyl-1-phenylpropan-1-ol was reacted to give title compound as a yellow oil. (1.2 g, 75%). ¹H NMR: (400 MHz, DMSO) δ 7.43-7.27 (m, 5H), 5.04 (d, J=8.8 Hz, 1H), 2.36-2.27 (m, 1H), 1.13 (d, J=6.4 Hz, 3H), 0.78 (d, J=6.4 Hz, 3H)

### Preparation 113: N⁵-ethyl-N²-methyl-3-(2-methyl-1-phenylpropoxy)-1H-pyrrole-2,5-dicarboxamide

Following the procedure in Example 1, Preparation 2, N⁵-ethyl-3-hydroxy-N⁵-methyl-1H-pyrrole-2,5-dicarboxamide and (1-bromo-2-methylpropyl) benzene were reacted to give title compound as off-white solid (0.037 g, 24%). ¹H NMR: (400 MHz, DMSO) δ 11.20 (s, 1H), 8.22 (t, J=5.2 Hz, 1H), 7.40-7.32 (m, 4H), 7.27-7.24 (m, 1H), 7.18-7.17 (m, 1H), 6.29 (s, 1H), 4.90 (d, J=7.2 Hz, 1H), 3.18 (m, 2H), 2.87 (d, J=4.8 Hz, 3H), 2.21-2.18 (m, 1H), 1.06-1.02 (m, 6H), 0.79 (t, J=6.8 Hz, 3H). m/z=342 [M−H]⁻.

### Example 78—N⁵-ethyl-N²-methyl-3-(1-(pyridazin-4-yl)ethoxy)-1H-pyrrole-2,5-dicarboxamide, Enantiomer A

### Example 79—N⁵-ethyl-N²-methyl-3-(1-(pyridazin-4-yl)ethoxy)-1H-pyrrole-2,5-dicarboxamide, Enantiomer B

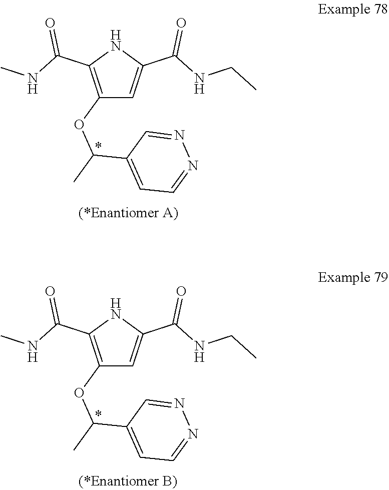

### Preparation 114: 1-(pyridazin-4-yl) ethanol

To a stirred mixture of 1-(pyridazin-4-yl) ethanone (2.00 g, 16.3 mmol) in MeOH (20.0 mL) was added NaBH₄ (923 mg, 24.4 mmol) at 0° C. The resulting mixture was stirred for 2 h at room temperature. The reaction mixture was filtered through a short pad of Celite. The pad was washed with MeOH (3×10.0 mL). The combined filtrate was concentrated under reduced pressure. The residue was purified by silica gel column chromatography, eluted with MeOH/EA (1:10) to afford the title compound as a yellow solid (1.30 g, 63.9%). LCMS: m/z=125 [M+H]⁺

### Preparation 115: Ethyl 5-(methylcarbamoyl)-4-(1-(pyridazin-4-yl)ethoxy)-1H-pyrrole-2-carboxylate

To a solution of ethyl 4-hydroxy-5-(methylcarbamoyl)-1H-pyrrole-2-carboxylate (678 mg, 3.19 mmol) and 1-(pyridazin-4-yl) ethanol (793 mg, 6.39 mmol) in THE (0.50 mL) was added PPh₃ (1.26 g, 4.79 mmol) at room temperature under nitrogen atmosphere. The resulting mixture was stirred for 30 min and followed by the addition of DIAD (969 mg, 4.79 mmol) at 0° C. The resulting mixture was stirred for 2 h at room temperature under nitrogen atmosphere. The resulting mixture was quenched with water (10.0 mL) and extracted with EtOAc (3×10 mL). The combined organic phases were washed with brine and dried over Na₂SO₄. After filtration, the filtrate was concentrated under reduced pressure. The residue was purified by silica gel column chromatography, eluted with MeOH/EA (1:10) to afford the title compound as a yellow oil (1.00 g, crude). LCMS: m/z=319 [M+H]⁺

### Preparation 116: 5-(methylcarbamoyl)-4-(1-(pyridazin-4-yl)ethoxy)-1H-pyrrole-2-carboxylic acid

To a stirred mixture of ethyl 5-(methylcarbamoyl)-4-(1-(pyridazin-4-yl)ethoxy)-1H-pyrrole-2-carboxylate (1.00 g, 3.14 mmol) in MeOH (8.00 mL) and H₂O (2.00 mL) was added LiOH (0.38 g, 15.7 mmol) at room temperature. The resulting mixture was stirred at 60° C. for 2 h. The resulting mixture was neutralized to pH 5 with 1 M aq. HCl and extracted with EtOAc (3×50 mL). The combined organic phases were washed with brine and dried over Na₂SO₄. After filtration, the filtrate was concentrated under reduced pressure. The residue was purified by silica gel column chromatography, eluted with EA/MeOH (10:1) to afford the title compound as a yellow oil (800 mg, 87.7%). LCMS: m/z=291 [M+H]⁺

### Preparation 117: N⁵-ethyl-N²-methyl-3-(1-(pyridazin-4-yl)ethoxy)-1H-pyrrole-2,5-dicarboxamide

To a stirred mixture of 5-(methylcarbamoyl)-4-(1-(pyridazin-4-yl)ethoxy)-1H-pyrrole-2-carboxylic acid (400 mg, 1.37 mmol) in DMF (5.00 mL) were added HATU (785 mg, 2.06 mmol), DIEA (356 mg, 2.75 mmol) and ethylamine hydrochloride (561 mg, 6.89 mmol) at room temperature. The resulting mixture was stirred at room temperature for 2 h. The resulting mixture was quenched with water (10.0 mL) and extracted with EA (3×10 mL). The combined organic phases were washed with brine and dried over Na₂SO₄. After filtration, the filtrate was concentrated under vacuum. The residue was purified by RP-HPLC^{H} to give the mixture of enantiomers (150 mg). The mixture was separated by Chiral Perp-HPLC^{J} to afford the enantiomer A (51.0 mg, 11.6%) and the enantiomer B (50.4 mg, 11.5%).

Example 78 (Enantiomer A): ¹H NMR (400 MHz, DMSO-d⁵) δ 11.29 (s, 1H), 9.37 (s, 1H), 9.22 (d, J=5.1 Hz, 1H), 8.20 (s, 1H), 7.74-7.73 (m, 1H), 7.28-7.27 (m, 1H), 6.38 (s, 1H), 5.52-5.50 (m, 1H), 3.19-3.16 (m, 2H), 2.86-2.85 (d, J=4.8 Hz, 3H) 1.64 (d, J=6.4 Hz, 3H), 1.06 (t, J=7.2 Hz, 3H). LCMS^{I}: m/z=318 [M+H]⁺. Chiral HPLC^{E} t_{R}: 0.902 min, ee %: 100%. The absolute configuration has not yet been assigned unambiguously but is believed to be the S enantiomer.

Example 79 (Enantiomer B): ¹H NMR (400 MHz, DMSO-d⁵) δ 11.29 (s, 1H), 9.37 (s, 1H), 9.22 (d, J=5.1 Hz, 1H), 8.20 (s, 1H), 7.74-7.73 (m, 1H), 7.28-7.27 (m, 1H), 6.38 (s, 1H), 5.52-5.50 (m, 1H), 3.19-3.16 (m, 2H), 2.86-2.85 (d, J=4.8 Hz, 3H) 1.64 (d, J=6.4 Hz, 3H), 1.06 (t, J=7.2 Hz, 3H). LCMS^{I}: m/z=318 [M+H]⁺; Chiral HPLC^{E} t_{R}: 1.862 min, ee %: 100%. The absolute configuration has not yet been assigned unambiguously but is believed to be the R enantiomer.

### Example 80—(S)-5-(1-isopropyl-1H-pyrazol-4-yl)-N-methyl-3-(1-phenylethoxy)-1H-pyrrole-2-carboxamide

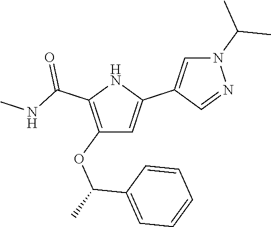 [SMILES: CNC(=O)c1[nH]c(-c2cnn(C(C)C)c2)cc1O[C@@H](C)c1ccccc1]

### Preparation 118: Ethyl (S)-5-(methylcarbamoyl)-4-(1-phenylethoxy)-1-((2-(trimethylsilyl)ethoxy)methyl)-1H-pyrrole-2-carboxylate

To a mixture of ethyl (S)-5-(methylcarbamoyl)-4-(1-phenylethoxy)-1H-pyrrole-2-carboxylate (5.50 g, 17.3 mmol) in DMF (60.0 mL) was added NaH (1.38 g, 34.7 mmol, 60% wt in mineral oil). The resulting mixture was stirred at 0° C. for 30 min and followed by addition of SEM-Cl (5.80 g, 34.0 mmol, 2.00 equiv). The resulting mixture was stirred at room temperature for additional 2 h. The resulting mixture was extracted with EtOAc (3×20.0 mL). The combined organic phases were washed with brine (10 mL) and dried over anhydrous Na₂SO₄. After filtration, the filtrate was concentrated under reduced pressure. The residue was purified by silica gel column chromatography, eluted with PE/EA (1:1) to afford the title compound as a yellow oil (8.00 g, 93.0%). LCMS: m/z=447 [M+H]⁺.

### Preparation 119: (S)-5-(methylcarbamoyl)-4-(1-phenylethoxy)-1-((2-(trimethylsilyl)ethoxy)methyl)-1H-pyrrole-2-carboxylic acid

To a stirred mixture of ethyl (S)-5-(methylcarbamoyl)-4-(1-phenylethoxy)-1-((2-(trimethylsilyl)ethoxy)methyl)-1H-pyrrole-2-carboxylate (8.00 g, 17.9 mmol) in MeOH (80.0 mL) and H₂O (20.0 mL) was added LiOH (2.15 g, 89.7 mmol) at room temperature. The resulting mixture was stirred at 60° C. for 2 h. The resulting mixture was concentrated under reduced pressure. The residue was purified by RP-HPLC^{I} to afford the title compound as a yellow oil (5.50 g, 73.3%). LCMS: m/z=419 [M+H]⁺.

### Preparation 120: (S)-5-bromo-N-methyl-3-(1-phenylethoxy)-1-((2-(trimethylsilyl)ethoxy)methyl)-1H-pyrrole-2-carboxamide

To a stirred mixture of (S)-5-(methylcarbamoyl)-4-(1-phenylethoxy)-1-((2-(trimethylsilyl)ethoxy)methyl)-1H-pyrrole-2-carboxylic acid (5.40 g, 12.9 mmol) and LiBr (3.36 g, 38.7 mmol) in THE (100 mL) was added PhI(OAc)₂ (4.99 g, 15.4 mmol) at room temperature under nitrogen atmosphere. The resulting mixture was stirred at room temperature under nitrogen atmosphere for 2 h. The resulting mixture was extracted with EtOAc (3×40.0 mL). The combined organic phases were washed with brine and dried over anhydrous Na₂SO₄. After filtration, the filtrate was concentrated under reduced pressure. The residue was purified by silica gel column chromatography, eluted with PE/EA (5:1) to afford the title compound as a brown-yellow oil (2.20 g, 37.6%). LCMS: m/z=455 [M+H]⁺.

### Preparation 121: (S)-5-(1-isopropyl-1H-pyrazol-4-yl)-N-methyl-3-(1-phenylethoxy)-1-((2-(trimethylsilyl)ethoxy)methyl)-1H-pyrrole-2-carboxamide

To a stirred mixture of (S)-5-bromo-N-methyl-3-(1-phenylethoxy)-1-((2-(trimethylsilyl)ethoxy)methyl)-1H-pyrrole-2-carboxamide (200 mg, 0.44 mmol) and 1-isopropylpyrazol-4-ylboronic acid (135 mg, 0.88 mmol) in dioxane (2.00 mL) and H₂O (0.50 mL) were added K₂CO₃ (121 mg, 0.88 mmol) and Pd(PPh₃)₄(50.9 mg, 0.04 mmol) at room temperature under nitrogen atmosphere. The resulting mixture was stirred at 100° C. under nitrogen atmosphere for 2 h. The resulting mixture was extracted with EtOAc (3×10.0 mL). The combined organic phases were washed with brine and dried over anhydrous Na₂SO₄. After filtration, the filtrate was concentrated under reduced pressure. The residue was purified by Prep-TLC (PE/EA 2:1) to afford the title compound as a yellow oil (100 mg, 46.9%). LCMS: m/z=483 [M+H]⁺.

### Preparation 122: (S)-5-(1-isopropyl-1H-pyrazol-4-yl)-N-methyl-3-(1-phenylethoxy)-1H-pyrrole-2-carboxamide

To a stirred mixture of (S)-5-(1-isopropyl-1H-pyrazol-4-yl)-N-methyl-3-(1-phenylethoxy)-1-((2-(trimethylsilyl)ethoxy)methyl)-1H-pyrrole-2-carboxamide (100 mg, 0.20 mmol) in THE (1.00 mL) was added tetrabutylazanium fluoride (108 mg, 0.41 mmol) at room temperature. The resulting mixture was stirred at 80° C. overnight. The resulting mixture was extracted with EtOAc (3×10.0 mL). The combined organic phases were washed with brine and dried over anhydrous Na₂SO₄. After filtration, the filtrate was concentrated under reduced pressure. The crude product (40 mg) was purified by Chiral Prep-HPLC^{I} to afford the title compound as a white solid (15 mg, 20.5%). ¹H NMR (400 MHz, DMSO-d₆) δ 11.04 (s, 1H), 8.11 (s, 1H), 7.76 (s, 1H), 7.46-7.44 (m, 2H), 7.38-7.33 (m, 2H), 7.28-7.26 (m, 1H), 6.94-6.92 (m, 1H), 6.01-6.00 (m, 1H), 5.36-5.33 (m, 1H), 4.43-4.39 (m, 1H), 2.84-2.83 (m, 3H), 1.62-1.60 (d, J=8.4 Hz, 3H), 1.39-1.37 (d, J=8.8 Hz, 6H). LCMS^{F}: m/z=353 [M+H]⁺; Chiral HPLC^{I} t_{R}: 0.857 min, ee %: 100%.

### Example 81—(S)-5-(1,3-dimethyl-1H-pyrazol-5-yl)-N-methyl-3-(1-phenylethoxy)-1H-pyrrole-2-carboxamide

### Example 82—(R)-5-(1,3-dimethyl-1H-pyrazol-5-yl)-N-methyl-3-(1-phenylethoxy)-1H-pyrrole-2-carboxamide

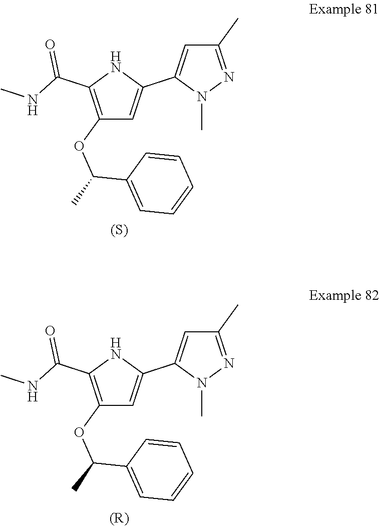

### Preparation 123: (S)-5-(1,3-dimethyl-1H-pyrazol-5-yl)-N-methyl-3-(1-phenylethoxy)-1-((2-(trimethylsilyl)ethoxy)methyl)-1H-pyrrole-2-carboxamide

To a stirred mixture of (S)-5-bromo-N-methyl-3-(1-phenylethoxy)-1-((2-(trimethylsilyl)ethoxy)methyl)-1H-pyrrole-2-carboxamide (500 mg, 1.10 mmol) and 2,5-dimethylpyrazol-3-ylboronic acid (308 mg, 2.20 mmol) in dioxane (4.00 mL) were added K₂CO₃ (304 mg, 2.20 mmol) and Pd(PPh₃)₄(127 mg, 0.11 mmol) at room temperature under nitrogen atmosphere. The resulting mixture was stirred for 4 h at 100° C. under nitrogen atmosphere. The resulting mixture was extracted with EtOAc (3×10.0 mL). The combined organic phases were washed with brine and dried over anhydrous Na₂SO₄. After filtration, the filtrate was concentrated under reduced pressure. The residue was purified by silica gel column chromatography, eluted with PE/EA (1:1) to afford the title compound as a yellow oil (300 mg, 58.0%). LCMS: m/z=467 [M+H]⁺

### Preparation 124: (S)- and (R)-5-(1,3-dimethyl-1H-pyrazol-5-yl)-N-methyl-3-(1-phenylethoxy)-1H-pyrrole-2-carboxamide

The solution of (S)-5-(1,3-dimethyl-1H-pyrazol-5-yl)-N-methyl-3-(1-phenylethoxy)-1-((2-(trimethylsilyl)ethoxy)methyl)-1H-pyrrole-2-carboxamide (300 mg, 0.64 mmol) in 1 M TBAF in THE (3.00 mL) was stirred at 80° C. for 4 h. The resulting mixture was extracted with EtOAc (3×15.0 mL). The organic phases were washed with brine and dried over anhydrous Na₂SO₄. After filtration, the filtrate was concentrated under reduced pressure. The residue was purified by Prep-TLC (PE/EA 2:1) to afford the product as the mixture of enantiomers (150 mg). Chiral HPLC analysis indicated a small amount of the (R) isomer was present due to epimerisation during the synthesis. The mixture was therefore separated by Chiral Prep-HPLC^{H} to afford the (S) enantiomer as a white solid (68.8 mg, 31.7%) and the (R) enantiomer as a white solid (20.2 mg, 8.90%).

Example 81: ¹H NMR (400 MHz, DMSO-d₆) δ 11.25 (s, 1H), 7.48-7.47 (m, 2H), 7.36-7.33 (m, 2H), 7.28-7.24 (m, 1H), 7.09-7.07 (m, 1H), 6.33 (s, 1H), 6.13-6.12 (m, 1H), 5.47-5.42 (m, 1H), 3.82-3.69 (m, 3H), 2.86-2.84 (m, 3H), 2.08 (s, 3H), 1.64-1.62 (m, 3H). LCMS^{F}: m/z=339 [M+H]⁺; Chiral HPLC^{H} t_{R}: 2.37 min, ee %: 100%.

Example 82: ¹H NMR (400 MHz, DMSO-d₆) δ 11.25 (s, 1H), 7.48-7.47 (m, 2H), 7.37-7.33 (m, 2H), 7.28-7.24 (m, 1H), 6.33 (s, 1H), 6.13-6.12 (m, 1H), 5.47-5.42 (m, 1H), 3.82-3.69 (m, 3H), 2.86-2.84 (m, 3H), 2.08 (s, 3H), 1.64-1.62 (m, 3H). LCMS^{F} m/z=339 [M+H]⁺, Chiral HPLC^{H} t_{R}: 2.78 min, ee %: 100%.

### Example 83—(S)—N-methyl-3-(1-phenylethoxy)-5-(1-(trifluoromethyl)-1H-pyrazol-4-yl)-1H-pyrrole-2-carboxamide

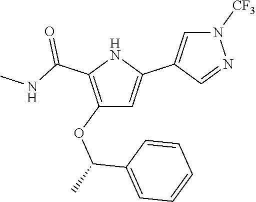 [SMILES: CNC(=O)c1[nH]c(-c2cnn(C(F)(F)F)c2)cc1O[C@@H](C)c1ccccc1]

### Preparation 125: (S)—N-methyl-3-(1-phenylethoxy)-5-(1-(trifluoromethyl)-1H-pyrazol-4-yl)-1-((2-(trimethylsilyl)ethoxy)methyl)-1H-pyrrole-2-carboxamide

To a stirred mixture of (S)-5-bromo-N-methyl-3-(1-phenylethoxy)-1-((2-(trimethylsilyl)ethoxy)methyl)-1H-pyrrole-2-carboxamide (400 mg, 0.88 mmol) and 4-(4,4,5,5-tetramethyl-1,3,2-dioxaborolan-2-yl)-1-(trifluoromethyl)pyrazole (462 mg, 1.76 mmol) in dioxane (4.00 mL) and H₂O (1.00 mL) were added K₂CO₃ (243 mg, 1.76 mmol) and Pd(PPh₃)₄(101 mg, 0.08 mmol) at room temperature under nitrogen atmosphere. The resulting mixture was stirred at 100° C. for 4 h. The resulting mixture was extracted with EtOAc (3×15.0 mL). The combined organic phases were washed with brine and dried over anhydrous Na₂SO₄. After filtration, the filtrate was concentrated under reduced pressure. The residue was purified by silica gel column chromatography, eluted with PE/EA (5:1) to afford the title compound as a yellow oil (300 mg, 66.8%). LCMS: m/z=509 [M+H]⁺

### Preparation 126: (S)—N-methyl-3-(1-phenylethoxy)-5-(1-(trifluoromethyl)-1H-pyrazol-4-yl)-1H-pyrrole-2-carboxamide

To a stirred mixture of (S)—N-methyl-3-(1-phenylethoxy)-5-(1-(trifluoromethyl)-1H-pyrazol-4-yl)-1-((2-(trimethylsilyl)ethoxy)methyl)-1H-pyrrole-2-carboxamide (300 mg, 0.59 mmol) in THE (3.00 mL) was added TBAF (308 mg, 1.18 mmol). The resulting mixture was stirred at 80° C. overnight. The resulting mixture was extracted with EtOAc (3×10.0 mL). The combined organic phases were washed with brine and dried over anhydrous Na₂SO₄. After filtration, the filtrate was concentrated under reduced pressure. The residue was purified by Prep-TLC (PE/EA 1:1) to afford the crude product (200 mg) which was further purified by Chiral Prep-HPLC® to afford the title compound as a white solid (87.2 mg, 39.0%). ¹H NMR (400 MHz, DMSO-d₆) δ 11.40 (s, 1H), 8.79 (s, 1H), 8.33 (s, 1H), 7.47-7.45 (m, 2H), 7.38-7.34 (m, 2H), 7.29-7.27 (s, 1H), 7.02-7.01 (m, 1H), 6.30 (s, 1H), 5.34-5.33 (m, 1H), 2.86-2.85 (d, J=4.8 Hz, 3H), 1.64-1.62 (d, J=6.4 Hz, 3H). LCMS^{G}: m/z=379 [M+H]⁺. Chiral HPLC® t_{R}: 5.56 min, ee %: 100%.

### Example 84—(S)—N-methyl-5-phenyl-3-(1-phenylethoxy)-1H-pyrrole-2-carboxamide

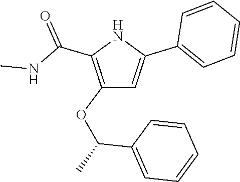 [SMILES: CNC(=O)c1[nH]c(-c2ccccc2)cc1O[C@@H](C)c1ccccc1]

### Preparation 127: (S)—N-methyl-5-phenyl-3-(1-phenylethoxy)-1-((2-(trimethylsilyl)ethoxy)methyl)-1H-pyrrole-2-carboxamide

To a stirred mixture of (S)-5-bromo-N-methyl-3-(1-phenylethoxy)-1-((2-(trimethylsilyl)ethoxy)methyl)-1H-pyrrole-2-carboxamide (400 mg, 0.88 mmol) and phenyl boronic acid (215 mg, 1.76 mmol) in dioxane (4.00 mL) and water (0.80 mL) at room temperature under nitrogen atmosphere were added K₂CO₃ (243 mg, 1.76 mmol) and Pd(PPh₃)₄(101 mg, 0.08 mmol). The resulting mixture was stirred at 100° C. for 4 h. The resulting mixture was extracted with EtOAc (3×15.0 mL). The combined organic phases were dried over anhydrous Na₂SO₄. After filtration, the filtrate was concentrated under reduced pressure. The residue was purified by silica gel column chromatography, eluted with PE/EA (4:1) to afford the title compound as a red oil (290 mg, 72.9%). LCMS: m/z=451 [M+H]⁺

### Preparation 128: (S)—N-methyl-5-phenyl-3-(1-phenylethoxy)-1H-pyrrole-2-carboxamide

To a stirred mixture of (S)—N-methyl-5-phenyl-3-(1-phenylethoxy)-1-((2-(trimethylsilyl)ethoxy)methyl)-1H-pyrrole-2-carboxamide (280 mg, 0.62 mmol) in THE (3.00 mL) was added TBAF (324 mg, 1.24 mmol) at room temperature. The resulting mixture was stirred at 80° C. overnight. The resulting mixture was extracted with EtOAc (3×10.0 mL). The combined organic phases were wash with brine (3×10.0 mL) and dried over anhydrous Na₂SO₄. After filtration, the filtrate was concentrated under reduced pressure. The residue was purified by Prep-TLC (PE/EA 1:1) to afford the crude product (150 mg) as a yellow solid. The mixture (150 mg) was further purified by Chiral Prep-HPLC^{R} to afford the title compound as a white solid (63.3 mg, 31.8%). ¹H NMR (400 MHz, DMSO-d₆) δ 11.23 (s, 1H), 7.71-7.69 (m, 2H), 7.49-7.47 (m, 2H), 7.37-7.26 (m, 5H), 7.20-7.18 (m, 1H), 7.07-7.06 (m, 1H), 6.34 (s, 1H), 5.45-5.43 (m, 1H) 2.87-2.85 (d, J=4.8 Hz, 3H) 1.64-1.62 (d, J=6.4 Hz, 3H). LCMS^{H}: m/z=321 [M+H]⁺, Chiral HPLC^{P} t_{R}: 2.21 min, ee %: 100%.

### Example 85—(S)-5-(2-fluorophenyl)-N-methyl-3-(1-phenylethoxy)-1H-pyrrole-2-carboxamide

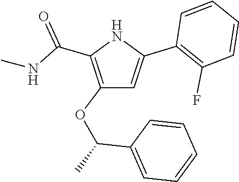 [SMILES: CNC(=O)c1[nH]c(-c2ccccc2F)cc1O[C@@H](C)c1ccccc1]

### Preparation 129: (S)-5-(2-fluorophenyl)-N-methyl-3-(1-phenylethoxy)-1-((2-(trimethylsilyl)ethoxy)methyl)-1H-pyrrole-2-carboxamide

To a stirred mixture of (S)-5-bromo-N-methyl-3-(1-phenylethoxy)-1-((2-(trimethylsilyl)ethoxy)methyl)-1H-pyrrole-2-carboxamide (350 mg, 0.772 mmol) and 2-fluorophenylboronic acid (216 mg, 1.54 mmol) in dioxane (4.00 mL) at room temperature under nitrogen atmosphere was added K₂CO₃ (213 mg, 1.54 mmol) and Pd(PPh₃)₄(89.2 mg, 0.077 mmol). The resulting mixture was stirred at 100° C. for 4 h. The resulting mixture was diluted with water (10.0 mL) and extracted with EtOAc (3×10.0 mL). The combined organic layers were washed with brine and dried over anhydrous Na₂SO₄. After filtration, the filtrate was concentrated under reduced pressure. The residue was purified by silica gel column chromatography, eluted with PE/EA (5:1) to afford the title compound as a white solid (106 mg, 29.3%). LCMS: m/z=469 [M+H]⁺

### Preparation 130: (S)-5-(2-fluorophenyl)-N-methyl-3-(1-phenylethoxy)-1H-pyrrole-2-carboxamide

The solution of (S)-5-(2-fluorophenyl)-N-methyl-3-(1-phenylethoxy)-1-((2-(trimethylsilyl)ethoxy)methyl)-1H-pyrrole-2-carboxamide (100 mg, 0.213 mmol, 1.00 equiv) in TBAF (1.00 mL, 1.00 mol/L in THF) was stirred at 80° C. for 15 h. The resulting mixture was concentrated under reduced pressure and diluted with water (5.00 mL). The resulting mixture was extracted with EtOAc (3×5.00 mL). The combined organic layers were washed with brine and dried over anhydrous Na₂SO₄. After filtration, the filtrate was concentrated under reduced pressure. The resulting mixture was concentrated under reduced pressure. The residue was purified by Prep-TLC (PE/EA 1:1) to afford the crude product as yellow solid (30 mg, 41.5%). The mixture was further purified by Chiral Prep-HPLC^{F} to afford the title compound as white solid (13.8 mg, 46%). ¹H NMR (400 MHz, Chloroform-d) δ 9.42 (s, 1H), 7.51-7.42 (m, 1H), 7.42-7.34 (m, 4H), 7.30-7.29 (m, 1H), 7.22-7.14 (m, 1H), 7.14-7.03 (m, 2H), 6.99-6.79 (m, 1H), 6.11 (d, J=2.6 Hz, 1H), 5.37-5.23 (m, 1H), 3.01 (d, J=4.9 Hz, 3H), 1.71 (d, J=6.5 Hz, 3H). LCMS^{H}: m/z=339 [M+H]⁺. Chiral HPLC^{K} t_{R}: 0.78 min, ee %: 100%.

### Example 86—(S)-5-(2,4-difluorophenyl)-N-methyl-3-(1-phenylethoxy)-1H-pyrrole-2-carboxamide

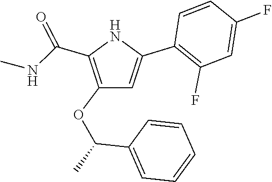 [SMILES: CNC(=O)c1[nH]c(-c2ccc(F)cc2F)cc1O[C@@H](C)c1ccccc1]

### Preparation 131: (S)-5-(2,4-difluorophenyl)-N-methyl-3-(1-phenylethoxy)-1-((2-(trimethylsilyl)ethoxy)methyl)-1H-pyrrole-2-carboxamide

To a stirred mixture of (S)-5-bromo-N-methyl-3-(1-phenylethoxy)-1-((2-(trimethylsilyl)ethoxy)methyl)-1H-pyrrole-2-carboxamide (300 mg, 0.662 mmol) and 2,4-difluorophenylboronic acid (209 mg, 1.32 mmol) in dioxane (4.00 mL) at room temperature under nitrogen atmosphere was added K₂CO₃ (183 mg, 1.32 mmol) and Pd(PPh₃)₄(76.5 mg, 0.0660 mmol). The resulting mixture was stirred at 100° C. for 2 h. The resulting mixture was diluted with water (10.0 mL) and extracted with EtOAc (3×20.0 mL). The combined organic layers were washed with brine and dried over anhydrous Na₂SO₄. After filtration, the filtrate was concentrated under reduced pressure. The residue was purified by silica gel column chromatography, eluted with PE/EA (6:1) to afford the title compound as yellow oil (120 mg, 37.2%). LCMS: m/z=487 [M+H]⁺

### Preparation 132: (S)-5-(2,4-difluorophenyl)-N-methyl-3-(1-phenylethoxy)-1H-pyrrole-2-carboxamide

A solution of (S)-5-(2,4-difluorophenyl)-N-methyl-3-(1-phenylethoxy)-1-((2-(trimethylsilyl)ethoxy)methyl)-1H-pyrrole-2-carboxamide (120 mg, 0.247 mmol) in 1 M TBAF in THE (1.00 mL) was stirred at 80° C. for 15 h. The resulting mixture was concentrated under reduced pressure. The residue was diluted with water (10.0 mL) and extracted with EtOAc (3×10.0 mL). The combined organic layers were washed with brine and dried over anhydrous Na₂SO₄. After filtration, the filtrate was concentrated under reduced pressure. The resulting mixture was concentrated under reduced pressure. The residue was purified by Prep-TLC (PE/EA 1:1) to afford the crude compound as yellow solid (40.0 mg, 45.5%). The mixture was further purified by Chiral Prep-HPLC^{G} to afford the title compound as white solid (7.40 mg, 18.5%). ¹H NMR (400 MHz, Chloroform-d) δ 9.42 (s, 1H), 7.45-7.33 (m, 5H), 7.32-7.29 (m, 1H), 6.87-6.83 (m, 3H), 6.03 (s, 1H), 5.30-5.28 (m, 1H), 3.10 (d, J=4.9 Hz, 3H), 1.80-1.67 (d, J=3.8 Hz, 3H). LCMS^{H}: m/z=357 [M+H]⁺. Chiral HPLC^{K} t_{R}: 0.75 min, ee %: 100%.

### Example 87—(S)-5-(4,5-dimethyl-1H-imidazol-2-yl)-N-methyl-3-(1-phenylethoxy)-1H-pyrrole-2-carboxamide

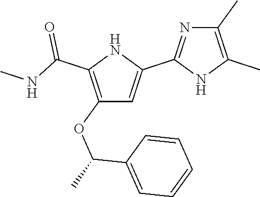

To a solution of (S)-5-formyl-N-methyl-3-(1-phenylethoxy)-1H-pyrrole-2-carboxamide (350 mg, 1.29 mmol) and butane-2,3-dione (1.33 g, 15.4 mmol) in MeOH (2.0 mL) was added NH₃·H₂O (3.50 mL, 89.9 mmol). The resulting mixture was stirred at 60° C. for 2 h. The mixture was concentrated under vacuum. The residue was purified by RP-HPLC^{I} to afford the crude product as a white solid (130 mg, 29.9%). The product (130 mg) was further purified by Chiral Prep-HPLC^{M} to afford the title product (75.7 mg, 58.2%). 1H NMR (400 MHz, DMSO-d₆) δ 11.60 (s, 1H), 10.76 (s, 1H), 7.50-7.41 (m, 2H), 7.40-7.32 (m, 2H), 7.30-7.23 (m, 1H), 7.09-6.91 (m, 1H), 6.11 (s, 1H), 5.50-5.30 (m, 1H), 2.87 (d, J=4.7 Hz, 3H), 2.21-1.91 (m, 6H), 1.61 (d, J=6.4 Hz, 3H). LCMS^{F}: m/z=339 [M+H]⁺. Chiral HPLC^{M} t_{R}: 1.32 min, ee %: 100%.

### Example 88—(S)—N-methyl-5-(1-methyl-1H-pyrazol-4-yl)-3-(1-phenylethoxy)-1H-pyrrole-2-carboxamide

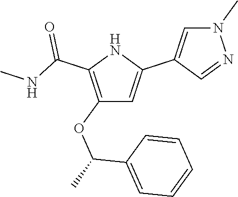

### Preparation 133: (S)—N-methyl-5-(1-methyl-1H-pyrazol-4-yl)-3-(1-phenylethoxy)-1-((2(trimethylsilyl)ethoxy)methyl)-1H-pyrrole-2-carboxamide

To a stirred mixture of (S)-5-bromo-N-methyl-3-(1-phenylethoxy)-1-((2-(trimethylsilyl)ethoxy)methyl)-1H-pyrrole-2-carboxamide (350 mg, 0.772 mmol) and 1-methyl-1H-pyrazol-4-ylboronic acid (194 mg, 1.54 mmol) in dioxane (4.00 mL) at room temperature under nitrogen atmosphere was added K₂CO₃ (213 mg, 1.54 mmol) and Pd(PPh₃)₄(89.2 mg, 0.0770 mmol). The resulting mixture was stirred at 100° C. for 2 h. The resulting mixture was diluted with water (10.0 mL) and extracted with EtOAc (3×30.0 mL). The combined organic layers were dried over anhydrous Na₂SO₄. After filtration, the filtrate was concentrated under reduced pressure. The residue was purified by silica gel column chromatography, eluted with PE/EA (5:1) to afford the title compound as yellow solid (180 mg, 51.2%). LCMS: m/z=455 [M+H]⁺.

### Preparation 134: (S)—N-methyl-5-(1-methyl-1H-pyrazol-4-yl)-3-(1-phenylethoxy)-1H-pyrrole-2-carboxamide

A solution of (S)—N-methyl-5-(1-methyl-1H-pyrazol-4-yl)-3-(1-phenylethoxy)-1-((2-(trimethylsilyl)ethoxy)methyl)-1H-pyrrole-2-carboxamide (170 mg, 0.374 mmol) in 1 M TBAF in THE (2.00 mL) was stirred at 80° C. for 15 h. The resulting mixture was concentrated under reduced pressure. The residue was diluted with water (10.0 mL). The resulting mixture was extracted with EtOAc (3×10.0 mL). The combined organic layers were dried over anhydrous Na₂SO₄. After filtration, the filtrate was concentrated under reduced pressure. The resulting mixture was concentrated under reduced pressure. The residue was purified by Prep-TLC (PE/EA 1:1) to afford the crude product as a yellow solid (30.0 mg, 37.4%). The mixture was further purified by Chiral Prep-HPLC^{S} to afford the title compound as white solid (13.6 mg, 45.3%). ¹H NMR (400 MHz, Chloroform-d) δ 9.26 (s, 1H), 7.57 (s, 1H), 7.52 (s, 1H), 7.37-7.35 (m, 4H), 7.32-7.29 (m, 1H), 6.87 (br, 1H), 5.72 (d, J=2.9 Hz, 1H), 5.28-5.23 (m, 1H), 3.88 (s, 3H), 3.00 (d, J=4.9 Hz, 3H), 1.74-1.68 (d, J=6.5 Hz, 3H). LCMS^{H}: m/z=325 [M+H]⁺. Chiral HPLC^{N} t_{R}: 1.81 min, ee %: 100%.

### Example 89—(S)-5-(1H-imidazol-2-yl)-N-methyl-3-(1-phenylethoxy)-1H-pyrrole-2-carboxamide

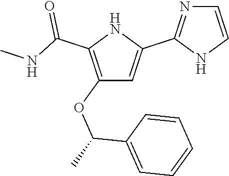 [SMILES: CNC(=O)c1[nH]c(-c2ncc[nH]2)cc1O[C@@H](C)c1ccccc1]

To a mixture of (S)-5-formyl-N-methyl-3-(1-phenylethoxy)-1H-pyrrole-2-carboxamide (400 mg, 1.47 mmol) and glyoxal (1.58 g, 17.60 mmol, 40 wt % in H₂O) in MeOH (2.00 mL) at 0° C. was added NH₃·H₂O (4.00 mL, 103 mmol) dropwise. The resulting mixture was stirred at room temperature overnight. The resulting mixture was concentrated under vacuum. The residue was purified by RP-HPLC^{O} to afford the product (40.0 mg, 8.7%) which was further purified by Chiral Prep-HPLC® to afford the title compound (15.7 mg, 39.3%). ¹H NMR (400 MHz, Methanol-d₄) δ 7.43-7.41 (m, 2H), 7.35-7.33 (m, 2H), 7.31-7.23 (m, 1H), 7.03 (s, 2H), 6.27 (s, 1H), 5.39-5.34 (m, 1H), 2.97 (s, 3H), 1.70 (d, J=6.4 Hz, 3H). LCMS^{F}: m/z=311 [M+H]⁺, Chiral HPLC^{G} t_{R}: 0.87 min, ee %: 100%.

### Example 90—(S)-5-(2,6-dimethylpyridin-4-yl)-N-methyl-3-(1-phenylethoxy)-1H-pyrrole-2-carboxamide

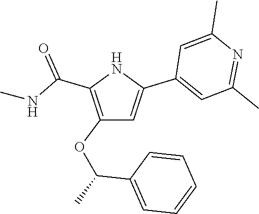

### Preparation 135: (S)-5-(2,6-dimethylpyridin-4-yl)-N-methyl-3-(1-phenylethoxy)-1-((2-(trimethylsilyl)ethoxy)methyl)-1H-pyrrole-2-carboxamide

To a stirred mixture of (S)-5-bromo-N-methyl-3-(1-phenylethoxy)-1-((2-(trimethylsilyl)ethoxy)methyl)-1H-pyrrole-2-carboxamide (500 mg, 1.10 mmol) and 2,6-dimethylpyridin-4-ylboronic acid (333 mg, 2.21 mmol) in dioxane (4.00 mL) at room temperature under nitrogen atmosphere were added K₂CO₃ (305 mg, 2.21 mmol) and Pd(PPh₃)₄(127 mg, 0.110 mmol). The resulting mixture was stirred at 100° C. for 2 h. The resulting mixture was diluted with water (10.0 mL). The resulting was extracted with EtOAc (3×20.0 mL). The combined organic layers were washed with brine and dried over anhydrous Na₂SO₄. After filtration, the filtrate was concentrated under reduced pressure. The residue was purified by silica gel column chromatography, eluted with PE/EA (4:1) to afford the title compound as a yellow oil (300 mg, 56.7%). LCMS: m/z=480 [M+H]⁺

### Preparation 136: (S)-5-(2,6-dimethylpyridin-4-yl)-N-methyl-3-(1-phenylethoxy)-1H-pyrrole-2-carboxamide

The solution of (S)-5-(2,6-dimethylpyridin-4-yl)-N-methyl-3-(1-phenylethoxy)-1-((2-(trimethylsilyl)ethoxy)methyl)-1H-pyrrole-2-carboxamide (300 mg, 0.643 mmol) in 1 M TBAF in THE (3.00 mL) at 80° C. for 15 h. The resulting mixture was concentrated under reduced pressure. The residue was diluted with water (10.0 mL) and extracted with EtOAc (3×30.0 mL). The combined organic layers were washed with brine and dried over anhydrous Na₂SO₄. After filtration, the filtrate was concentrated under reduced pressure. The resulting mixture was concentrated under reduced pressure. The residue was purified by Prep-TLC (PE/EA 1:1) to afford the product as yellow solid (120 mg, 40.0%). The mixture was further purified by Chiral Prep-HPLC^{P} to afford the title compound as white solid (72.2 mg, 60.1%). ¹H NMR (400 MHz, DMSO-d⁶) δ 11.48 (s, 1H), 7.52-7.45 (m, 2H), 7.42 (s, 2H), 7.36-7.34 (m, 2H), 7.31-7.22 (m, 1H), 7.13-7.12 (m, 1H), 6.58 (s, 1H), 5.44 (q, J=6.4 Hz, 1H), 2.87 (d, J=4.7 Hz, 3H), 2.37 (s, 6H), 1.64 (d, J=6.4 Hz, 3H). LCMS^{E} m/z=350 [M+H]⁺, Chiral HPLC^{F}t_{R}: 1.90 min, ee %: 100%.

### Example 91—N⁵-(4-hydroxycyclohexyl)-N²-methyl-3-(1-(pyridazin-4-yl)ethoxy)-1H-pyrrole-2,5-dicarboxamide, Isomer A

### Example 92—N⁵-(4-hydroxycyclohexyl)-N²-methyl-3-(1-(pyridazin-4-yl)ethoxy)-1H-pyrrole-2,5-dicarboxamide, Isomer B

### Example 93—N⁵-(4-hydroxycyclohexyl)-N²-methyl-3-(1-(pyridazin-4-yl)ethoxy)-1H-pyrrole-2,5-dicarboxamide, Isomer C

### Example 94—N⁵-(4-hydroxycyclohexyl)-N²-methyl-3-(1-(pyridazin-4-yl)ethoxy)-1H-pyrrole-2,5-dicarboxamide, Isomer D

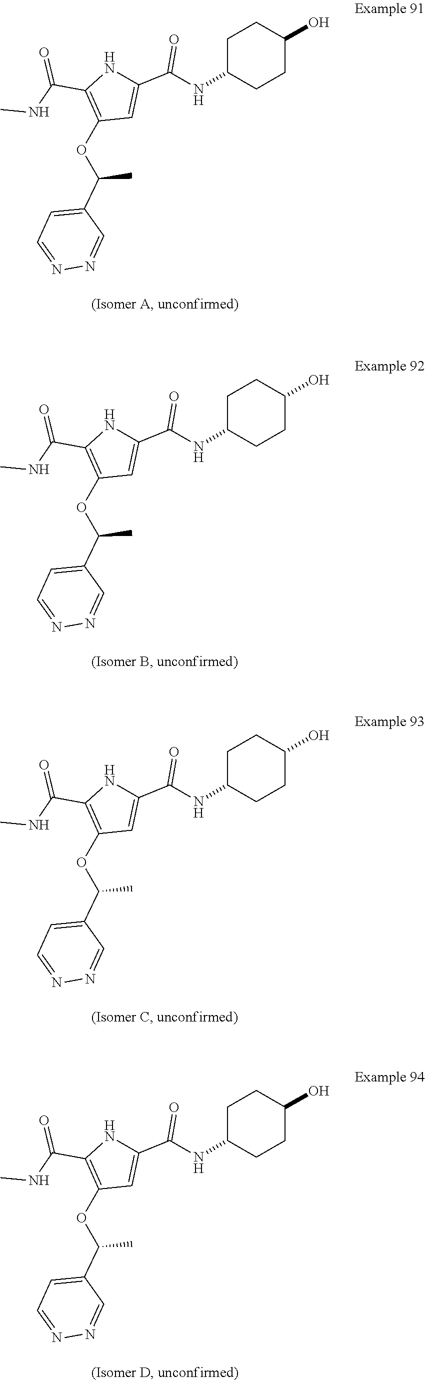 [SMILES: CNC(=O)c1[nH]c(C(=O)N[C@H]2CC[C@H](O)CC2)cc1O[C@@H](C)c1ccnnc1 ; CNC(=O)c1[nH]c(C(=O)N[C@H]2CC[C@@H](O)CC2)cc1O[C@@H](C)c1ccnnc1 ; CNC(=O)c1[nH]c(C(=O)N[C@H]2CC[C@@H](O)CC2)cc1O[C@H](C)c1ccnnc1 ; CNC(=O)c1[nH]c(C(=O)N[C@H]2CC[C@H](O)CC2)cc1O[C@H](C)c1ccnnc1]

To a stirred mixture of (S)-5-(methylcarbamoyl)-4-(1-(pyridazin-4-yl)ethoxy)-1H-pyrrole-2-carboxylic acid (600 mg, 2.07 mmol) in DMF (10.0 mL) were added HATU (1.57 g, 4.13 mmol), DIEA (401 mg, 3.10 mmol) and 4-aminocyclohexan-1-ol (476 mg, 4.13 mmol, mixture of cis and trans) at room temperature. The resulting mixture was stirred at room temperature for 2 h. The reaction mixture was concentrated under vacuum. The residue was purified by Prep-HPLC^{I} to afford product (400 mg, crude) as a light-yellow solid. The mixture was separated by Chiral Prep-HPLC^{I} to afford Example 91 (109.7 mg, 13.7%, light-yellow solid), Example 92 (53.8 mg, 6.7%, light-yellow solid) and a mixture of compounds. The mixture was further purified by Chiral Prep-HPLC^{K} to afford Example 93 (46.7 mg, 5.8%, yellow solid) and Example 94 (99.1 mg, 12.4%, yellow solid). The absolute configuration (S or R) and the relative geometries (cis/trans) have not been assigned unambiguously.

Example 91: ¹H NMR (400 MHz, CDCl₃) δ 10.59 (s, 1H), 9.20-9.14 (m, 2H), 7.44-7.42 (m, 1H), 6.86-6.76 (m, 2H), 6.19 (s, 1H), 5.33-5.29 (m, 1H), 3.97-3.88 (m, 2H), 3.06-2.99 (m, 3H), 1.74-1.72 (m, 4H), 1.72-1.59 (m, 7H). LCMS' m/z=388 [M+H]⁺. Chiral HPLC t_{R}: 1.20 min. ee %: 100%.

Example 92: ¹H NMR (400 MHz, CDCl₃) δ 10.10 (s, 1H), 9.22-9.18 (m, 2H), 7.45-7.43 (m, 1H), 6.78-6.77 (m, 1H), 6.03 (s, 1H), 5.95-5.94 (m, 1H), 5.30-5.25 (m, 1H), 3.91-3.81 (m 1H), 3.60-3.52 (m, 1H), 3.02 (s, 3H), 2.01-1.95 (m, 4H), 1.75-1.74 (m, 3H), 1.44-1.35 (m, 2H), 1.26-1.16 (m, 2H). LCMS^{I}: m/z=388 [M+H]⁺; Chiral HPLC^{I} t_{R}: 2.43 min, ee %: 100%.

Example 93: ¹H NMR (400 MHz, CDCl₃) δ 9.83 (s, 1H), 9.24-9.20 (m, 2H), 7.46-7.44 (m, 1H), 6.73-6.72 (m, 1H), 5.83-5.82 (m, 1H), 5.62-5.60 (m, 1H), 5.27-5.26 (m, 1H), 3.88-3.86 (m, 1H), 3.63-3.59 (m, 1H), 3.03-3.02 (m, 3H), 2.04-1.98 (m, 4H), 1.76-1.74 (m, 3H), 1.43-1.37 (m, 2H), 1.26-1.23 (m, 2H). LCMS^{E}: m/z=388 [M+H]⁺, Chiral HPLC^{E} t_{R}: 0.84 min, ee %: 100%.

Example 94: ¹H NMR (400 MHz, CDCl₃) δ 10.32 (s, 1H), 9.24-9.18 (m, 2H), 7.46-7.45 (m, 1H), 6.81-6.79 (m, 1H), 6.39 (br, 1H), 6.09 (s, 1H), 5.34-5.29 (m, 1H), 3.98-3.91 (m, 2H), 3.04-3.03 (m, 3H), 1.82 (s, 4H), 1.75-1.25 (m, 7H). LCMS^{E} m/z=388 [M+H]⁺, Chiral HPLC^{E} t_{R}: 1.37 min, ee %: 100%.

### Example 95—Racemic N⁵-ethyl-N²-methyl-3-(1-phenylethoxy)-1H-pyrrole-2,5-dicarboxamide

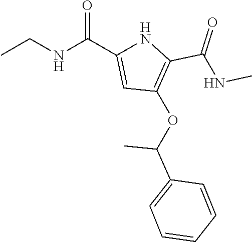 [SMILES: CCNC(=O)c1cc(OC(C)c2ccccc2)c(C(=O)NC)[nH]1]

### Preparation 137: Ethyl 4-bromo-5-formyl-1H-pyrrole-2-carboxylate

To a solution of DMF (9.49 mL, 123 mmol) in DCM (100 mL) was added POCl₃ (16.0 g, 105 mmol) at 0° C. The resulting mixture was stirred for additional 10 min at 0° C. To the above mixture was added ethyl 4-bromo-1H-pyrrole-2-carboxylate (7.64 g, 35.0 mmol). The resulting mixture was stirred for additional 24 h at room temperature. The mixture was concentrated under reduced pressure. The residue was diluted with water (100 mL). The aqueous layer was extracted with DCM (3×100 mL). The combined organic layers were washed with brine, dried over anhydrous Na₂SO₄. After filtration, the filtrate was concentrated under reduced pressure. The residue was purified by silica gel column chromatography, eluted with PE/EA (5:1) to afford ethyl 4-bromo-5-formyl-1H-pyrrole-2-carboxylate (8.00 g, 93%) as a white solid. LCMS: m/z=248 [M+H]⁺.

### Preparation 138: 3-Bromo-5-(ethoxycarbonyl)-1H-pyrrole-2-carboxylic acid

To a solution of ethyl 4-bromo-5-formyl-1H-pyrrole-2-carboxylate (10.0 g, 40.6 mmol) in DMSO (50.0 mL) was added NaH₂PO₄ (4.88 g, 40.6 mmol) in H₂O (25.0 mL) at 0° C. To the above mixture was added NaClO₂ (1.47 g, 163 mmol) in H₂O (25.0 mL) dropwise. The resulting mixture was stirred overnight at room temperature. The mixture was acidified to pH 2 with aq. HCl (1 N) until the white solids were precipitated. The precipitated solids were collected by filtration and washed with water. The filter cake was dried to give 3-bromo-5-(ethoxycarbonyl)-1H-pyrrole-2-carboxylic acid (10.0 g, 94%) as a white solid. LCMS: m/z=264 [M+H]⁺.

### Preparation 139: Ethyl 4-bromo-5-(methylcarbamoyl)-1H-pyrrole-2-carboxylate Intermediate 1

To a solution of 3-bromo-5-(ethoxycarbonyl)-1H-pyrrole-2-carboxylic acid (8.00 g, 30.5 mmol) in DMF (150 mL) was added CDI (9.90 g, 61.1 mmol) and DMAP (0.16 g, 1.33 mmol). The mixture was stirred for 3 h. To the above mixture was added 2 M methylamine in THE (61.1 mL, 122.2 mmol). The resulting mixture was stirred for 4 h at room temperature. The reaction was quenched by the addition of H₂O (50 mL). The resulting mixture was extracted with EtOAc (3×100 mL). The combined organic layers were washed with brine (2×50 mL), dried over anhydrous Na₂SO₄. After filtration, the filtrate was concentrated under reduced pressure. The aqueous layer was extracted with EtOAc (3×10 mL). The combined organic phases were dried over Na₂SO₄, after filtration, the filtrate was concentrated under vacuum. The residue was purified by silica gel column chromatography, eluted with PE/EA (5/1) to afford ethyl 4-bromo-5-(methylcarbamoyl)-1H-pyrrole-2-carboxylate (7.00 g, 83%) as a white solid. LCMS: m/z=277 [M+H]⁺.

### Preparation 140: 4-Bromo-5-(methylcarbamoyl)-1H-pyrrole-2-carboxylic acid

To a solution of ethyl 4-bromo-5-(methylcarbamoyl)-1H-pyrrole-2-carboxylate (6.00 g, 22.9 mmol) in MeOH (48.0 mL) and H₂O (12.0 mL) was added with LiOH (2.74 g, 114 mmol). The mixture was stirred for overnight at 60° C. The mixture was acidified to pH 2 with aq. HCl (1 N). The product was precipitated by the addition of water. This resulted in 4-bromo-5-(methylcarbamoyl)-1H-pyrrole-2-carboxylic acid (4.50 g, 80%) as a white solid. LCMS: m/z=249 [M+H]⁺.

### Preparation 141: 3-Bromo-N⁵-ethyl-N²-methyl-1H-pyrrole-2,5-dicarboxamide

To a solution of 4-bromo-5-(methylcarbamoyl)-1H-pyrrole-2-carboxylic acid (3.00 g, 12.1 mmol) in DMF (30 mL) was added CDI (3.94 g, 24.3 mmol). The mixture was stirred for 2 h at room temperature. To the above mixture was added EtNH₂ (1.09 g, 24.3 mmol). The resulting mixture was stirred for additional 1 h at room temperature. The resulting mixture was extracted with EtOAc (3×100 mL). The combined organic layers were washed with brine (2×50 mL), dried over anhydrous Na₂SO₄. After filtration, the filtrate was concentrated under reduced pressure. The residue was purified by reversed phase flash chromatography (C18 column; elution with 10-100% ACN in water with 0.1% TFA) to afford 3-bromo-N⁵-ethyl-N²-methyl-1H-pyrrole-2,5-dicarboxamide (2.80 g, 84%) as a white solid. LCMS: m/z=275 [M+H]⁺.

### Preparation 142: N⁵-ethyl-N²-methyl-3-(4,4,5,5-tetramethyl-1,3,2-dioxaborolan-2-yl)-1H-pyrrole-2,5-dicarboxamide

To a stirred mixture of 3-bromo-N⁵-ethyl-N²-methyl-1H-pyrrole-2,5-dicarboxamide (500 mg, 1.82 mmol) and bis(pinacolato)diboron (2.78 g, 10.9 mmol) in 1,4-dioxane (10.0 mL) were added KOAc (716 mg, 7.30 mmol) and Pd(dppf)Cl₂ (133 mg, 0.182 mmol) at room temperature under nitrogen atmosphere. The resulting mixture was stirred overnight at 60° C. under nitrogen atmosphere. The reaction was quenched with water at room temperature. The resulting mixture was extracted with EtOAc (3×15 mL). The combined organic layers were washed with water and brine, dried over anhydrous Na₂SO₄. After filtration, the filtrate was concentrated under reduced pressure. The residue was purified by reversed phase flash chromatography (C18 column; elution with 5-30% ACN in water with 0.1% FA) to afford N⁵-ethyl-N²-methyl-3-(4,4,5,5-tetramethyl-1,3,2-dioxaborolan-2-yl)-1H-pyrrole-2,5-dicarboxamide (420 mg, 72%) as an off-white solid. LCMS: m/z=322 [M+H]⁺.

### Preparation 143: N-ethyl-3-hydroxy-N²-methyl-1H-pyrrole-2,5-dicarboxamide Intermediate 2

To a stirred solution of N⁵-ethyl-N²-methyl-3-(4,4,5,5-tetramethyl-1,3,2-dioxaborolan-2-yl)-1H-pyrrole-2,5-dicarboxamide (200 mg, 0.623 mmol) in THE (1.50 mL) was added H₂O₂ (339 mg, 9.97 mmol) dropwise at 0° C. under nitrogen atmosphere. The resulting mixture was stirred for 2 h at room temperature. The reaction was quenched with aqueous NaOH at 0° C. The mixture was acidified to pH 3 with aq. HCl (1 N). The residue was purified by reversed phase flash chromatography (C18 column; elution with 5-30% ACN in water with 0.1% FA) to afford Intermediate 2 (54 mg, 41%) as an off-white solid. LCMS: m/z=212 [M+H]⁺.

### Preparation 144: Racemic N⁵-ethyl-N²-methyl-3-(1-phenylethoxy)-1H-pyrrole-2,5-dicarboxamide

To a mixture of 18-Crown-6 (3.75 mg, 0.0140 mmol) and t-BuOK (25.5 mg, 0.227 mmol) in THE (1.0 mL) was added Intermediate 2 (30.0 mg, 0.142 mmol) and (1-bromoethyl)benzene (36.8 mg, 0.20 mmol) at 0° C. under nitrogen atmosphere. The resulting mixture was stirred overnight at room temperature. The residue was purified by Prep-HPLC^{A}; to afford racemic N⁵-ethyl-N²-methyl-3-(1-phenylethoxy)-1H-pyrrole-2,5-dicarboxamide (9 mg, 20%) as a white solid. ¹H NMR (400 MHz, Methanol-d⁴) δ 7.49-7.20 (m, 5H), 6.39 (s, 1H), 5.32 (q, J=6.5 Hz, 1H), 3.30-3.26 (m, 2H), 2.99-2.92 (m, 3H), 1.68 (d, J=6.5 Hz, 3H), 1.15 (t, J=7.3 Hz, 3H). LCMS^{A}: m/z=314 [M−H]⁻.

### Example 96—Racemic N⁵-ethyl-N²-methyl-3-(1-(pyridin-2-yl)ethoxy)-1H-pyrrole-2,5-dicarboxamide

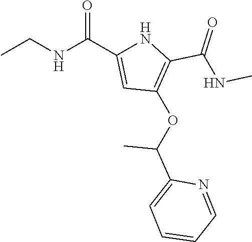 [SMILES: CCNC(=O)c1cc(OC(C)c2ccccn2)c(C(=O)NC)[nH]1]

Synthesised according to method described for Example 95 (Preparation 144) using (1-chloroethyl)pyridine as starting material. The residue was purified by Prep-HPLC^{B} to afford racemic N⁵-ethyl-N⁵-methyl-3-(1-(pyridin-2-yl)ethoxy)-1H-pyrrole-2,5-dicarboxamide (10 mg, 9%) as a white solid. ¹H NMR (400 MHz, Methanol-d₄) δ 8.63-8.48 (m, 1H), 7.89-7.79 (m, 1H), 7.51 (d, J=7.9 Hz, 1H), 7.41-7.28 (m, 1H), 6.38 (s, 1H), 5.38 (q, J=6.5 Hz, 1H), 3.31 (q, J=7.3, 2H), 2.97 (s, 3H), 1.71 (d, J=6.5 Hz, 3H), 1.15 (t, J=7.3 Hz, 3H). LCMS^{B}: m/z=317 [M+H]⁺.

### Example 97—N⁵-(trans-4-hydroxycyclohexyl)-N⁵-methyl-3-(1-phenylethoxy)-1H-pyrrole-2,5-dicarboxamide, Enantiomer A

### Example 98—N⁵-(trans-4-hydroxycyclohexyl)-N²-methyl-3-(1-phenylethoxy)-1H-pyrrole-2,5-dicarboxamide, Enantiomer B

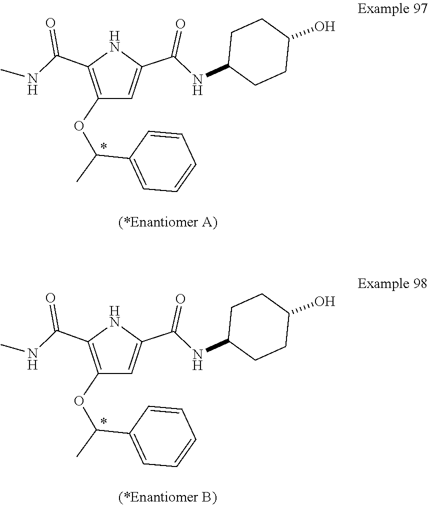 [SMILES: CNC(=O)c1[nH]c(C(=O)N[C@H]2CC[C@H](O)CC2)cc1OC(C)c1ccccc1 ; CNC(=O)c1[nH]c(C(=O)N[C@H]2CC[C@H](O)CC2)cc1OC(C)c1ccccc1]

### Preparation 145: Ethyl 5-(methylcarbamoyl)-4-(4,4,5,5-tetramethyl-1,3,2-dioxaborolan-2-yl)-1H-pyrrole-2-carboxylate

A mixture of Intermediate 1 (2.00 g, 7.27 mmol), bis(pinacolato)diboron (11.1 g, 43.6 mmol), XPhos Pd G3 (620 mg, 0.727 mmol) and KOAc (2.84 g, 29.0 mmol) in 1,4-dioxane (30 mL) was stirred for overnight at 60° C. under nitrogen atmosphere. The reaction was quenched by the addition of water (10 mL) at 0° C. The resulting mixture was extracted with EtOAc (3×20 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EA (5:1) to afford ethyl 5-(methylcarbamoyl)-4-(4,4,5,5-tetramethyl-1,3,2-dioxaborolan-2-yl)-1H-pyrrole-2-carboxylate (1.3 g, 56%) as a white solid. LCMS: m/z=323 [M+H]⁺.

### Preparation 146: Ethyl 4-hydroxy-5-(methylcarbamoyl)-1H-pyrrole-2-carboxylate Intermediate 3

A solution of ethyl 5-(methylcarbamoyl)-4-(4,4,5,5-tetramethyl-1,3,2-dioxaborolan-2-yl)-1H-pyrrole-2-carboxylate (1.30 g, 4.04 mmol) in THE (30 mL) was treated with H₂O₂(aq. 30% wt) (4.39 g, 129 mmol) in portions at 0° C. The resulting mixture was stirred for 10 min at room temperature. The residue was purified by reverse phase flash chromatography (C18 column, 10-50% MeCN in water with 0.1% TFA) to afford ethyl 4-hydroxy-5-(methylcarbamoyl)-1H-pyrrole-2-carboxylate (600 mg, 70%) as a white solid. LCMS: m/z=213 [M+H]⁺.

### Preparation 147: Racemic ethyl 5-(methylcarbamoyl)-4-(1-phenylethoxy)-1H-pyrrole-2-carboxylate

To a stirred mixture of 18-Crown-6 (74.7 mg, 0.283 mmol) and t-BuOK (508 mg, 4.52 mmol) in THE (10 mL) were added ethyl 4-hydroxy-5-(methylcarbamoyl)-1H-pyrrole-2-carboxylate (600 mg, 2.82 mmol) in THE (10 mL) and (1-bromoethyl)benzene (733 mg, 3.96 mmol) dropwise at 0° C. under nitrogen atmosphere. The resulting mixture was stirred overnight at room temperature under nitrogen atmosphere. The reaction was quenched by the addition of water (10 mL) at 0° C. The resulting mixture was extracted with EtOAc (3×20 mL). The combined organic layers were washed with brine (2×20 mL), dried over anhydrous Na₂SO₄. After filtration, the filtrate was concentrated under reduced pressure. The residue was purified by reverse phase flash chromatography (C18 column, 10-100% MeCN in water with 0.1% TFA) to afford ethyl 5-(methylcarbamoyl)-4-(1-phenylethoxy)-1H-pyrrole-2-carboxylate (600 mg, 70%) as a white solid. LCMS: m/z=317 [M+H]⁺.

### Preparation 148: Racemic 5-(methylcarbamoyl)-4-(1-phenylethoxy)-1H-pyrrole-2-carboxylic acid

A solution of racemic ethyl 5-(methylcarbamoyl)-4-(1-phenylethoxy)-1H-pyrrole-2-carboxylate (600 mg, 1.90 mmol) in CH₃OH (8.0 mL) and H₂O (2.0 mL) was treated with LiOH·H₂O (398 mg, 9.49 mmol) for 4 h at 60° C. The mixture was allowed to cool down to room temperature. The mixture was acidified to pH 2 with aq. HCl (1 N). The resulting mixture was extracted with EtOAc (4×20 mL). The combined organic layers were washed with brine (2×10 mL), dried over anhydrous Na₂SO₄. After filtration, the filtrate was concentrated under reduced pressure to afford racemic 5-(methylcarbamoyl)-4-(1-phenylethoxy)-1H-pyrrole-2-carboxylic acid (510 mg, crude) as a light yellow solid which was used directly in the following step. LCMS: m/z=289 [M+H]⁺.

### Preparation 149: N⁵-(trans-4-hydroxycyclohexyl)-N²-methyl-3-(1-phenylethoxy)-1H-pyrrole-2,5-dicarboxamide, Enantiomer A and B

A solution of racemic 5-(methylcarbamoyl)-4-(1-phenylethoxy)-1H-pyrrole-2-carboxylic acid (200 mg, 0.69 mmol) in DMF (5 mL) was treated with CDI (225 mg, 1.39 mmol) for 2 h at room temperature. To the above mixture was added trans-4-aminocyclohexan-1-ol (160 mg, 1.39 mmol). The resulting mixture was stirred for additional 4 h at room temperature. The reaction was quenched with water at room temperature. The resulting mixture was extracted with EtOAc (3×30 mL). The combined organic layers were washed with brine (2×10 mL), dried over anhydrous Na₂SO₄. After filtration, the filtrate was concentrated under reduced pressure. The crude product was purified by Prep-HPLC^{C} to afford product (170 mg, 48.6%) as a white solid which was separated by Chiral-Prep-HPLC^{A} to afford enantiomer A (R_{T} 9.8 min, Example 97, 49 mg, 18%) as a white solid and enantiomer B (R_{T} 12.9 min, Example 98, 56.0 mg, 21%) as a white solid.

Example 97 (Enantiomer A)¹H NMR (400 MHz, MeOH-d): δ 7.44-7.27 (m, 5H), 6.45 (s, 1H), 5.38-5.31 (m, 1H), 3.75 (s, 1H), 3.55-3.54 (m, 1H), 2.99-2.97 (m, 3H), 1.97-1.96 (m, 4H), 1.72-1.69 (m, 3H), 1.45-1.30 (m, 4H). LCMS: m/z=386 [M+H]⁺. Chiral HPLC^{A}: R_{T} 1.22 min, ee %>99%. The absolute configuration of Example 97 was confirmed to be (S) by re-synthesis using (S)-5-(methylcarbamoyl)-4-(1-phenylethoxy)-1H-pyrrole-2-carboxylic acid (synthesis of which is described in Example 105, Preparation 151) and trans-4-aminocyclohexan-1-ol using the method described in Example 78/79, Preparation 117 and subsequent purification. The retention times of this sample, compared with a sample of Example 97, Enantiomer A were identical as shown by providing a single peak when passed through the column as a mixture with Example 97, Enantiomer A.

Example 98 (Enantiomer B)¹H NMR (400 MHz, MeOH-d): δ 7.44-7.29 (m, 5H), 6.45 (s, 1H), 5.36-5.34 (m, 1H), 3.76-3.74 (m, 1H), 3.56-3.51 (m, 1H), 2.99-2.97 (m, 3H), 1.97-1.94 (m, 4H), 1.72-1.69 (m, 3H), 1.45-1.31 (m, 4H). LCMS: m/z=386 [M+H]⁺. Chiral HPLC^{A}: R_{T} 1.57 min, ee %>99%. The absolute configuration of Example 98 is believed to be (R) after confirmation that Example 97 is (S) as described above.

### Example 99—N⁵-(trans-4-methoxycyclohexyl)-N²-methyl-3-(1-phenylethoxy)-1H-pyrrole-2,5-dicarboxamide, Enantiomer A

### Example 100—N⁵-(trans-4-methoxycyclohexyl)-N²-methyl-3-(1-phenylethoxy)-1H-pyrrole-2,5-dicarboxamide, Enantiomer B

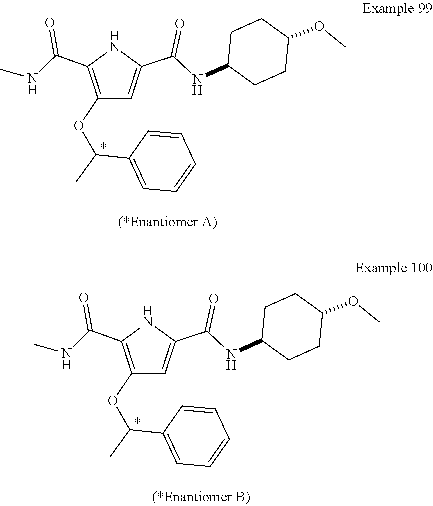

Synthesised according to method described in Example 97 and Example 98 (Preparation 149) using trans-4-methoxycyclohexan-1-amine as starting material. The crude product was purified by Prep-HPLC^{D} and separated via Chiral-Prep-HPLC^{B} to afford Enantiomer A (R_{T} 6.5 min, Example 99, 51 mg, 18%) as a white solid and Enantiomer B (R_{T} 8.3 min, Example 100, 50 mg, 18%) as a white solid.

Example 99 (Enantiomer A)¹H NMR (400 MHz, MeOH-d₄) δ 7.43-7.27 (m, 5H), 6.45 (s, 1H), 5.38-5.31 (m, 1H), 3.79-3.76 (m, 1H), 3.76-3.56 (m, 3H), 3.36-3.09 (m, 1H), 3.12-2.97 (m, 3H), 2.12-2.09 (m, 2H), 1.98-1.90 (m, 2H), 1.71-1.69 (m, 3H), 1.50-1.20 (m, 4H). LCMS^{D}: m/z=400 [M+H]⁺. Chiral HPLC^{B}: R_{T} 0.81 min, ee %>99%. The absolute configuration of Example 99 has not yet been assigned unambiguously but is believed to be the S-enantiomer.

Example 100 (Enantiomer B)¹H NMR (400 MHz, MeOH-d) δ 7.44-7.27 (m, 5H), 6.45 (s, 1H), 5.38-5.31 (m, 1H), 3.80-3.73 (m, 1H), 3.36-3.32 (m, 3H), 3.32-2.99 (m, 1H), 2.98 (s, 3H), 2.13-2.09 (m, 2H), 1.98-1.91 (m, 2H), 1.72-1.67 (m, 3H), 1.43-1.27 (m, 4H). LCMS^{D}: m/z=400 [M+H]⁺. Chiral HPLC^{B}: R_{T} 0.98 min, ee %>99%. The absolute configuration of Example 100 has not yet been assigned unambiguously but is believed to be the R-enantiomer.

### Example 101—N⁵-cyclobutyl-N²-methyl-3-(1-phenylethoxy)-1H-pyrrole-2,5-dicarboxamide, Enantiomer A

### Example 102—N⁵-cyclobutyl-N²-methyl-3-(1-phenylethoxy)-1H-pyrrole-2,5-dicarboxamide, Enantiomer B

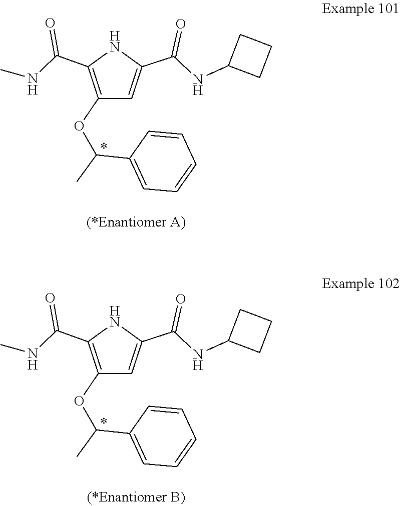 [SMILES: CNC(=O)c1[nH]c(C(=O)NC2CCC2)cc1OC(C)c1ccccc1 ; CNC(=O)c1[nH]c(C(=O)NC2CCC2)cc1OC(C)c1ccccc1]

Synthesised according to method described in Example 97 and Example 98 (Preparation 149) using cyclobutanamine as starting material. The crude product was purified by Prep-HPLC^{E} and further separated by Chiral-Prep-HPLC^{C} to afford Enantiomer A (R_{T} 17.0 min, Example 101, 50 mg, 24%) as a white solid and Enantiomer B (R_{T} 30.5 min, Example 102, 50 mg, 24%) as a white solid.

Example 101 (Enantiomer A)¹H NMR (400 MHz, MeOH-d₄) δ 7.43-7.29 (m, 5H), 6.46 (s, 1H), 5.38-5.32 (m, 1H), 4.46-4.35 (m, 1H), 2.98 (s, 3H), 2.33-2.26 (m, 2H), 2.11-1.97 (m, 2H), 1.80-1.72 (m, 5H). LCMS^{D}: m/z=340 [M−H]⁻. Chiral HPLC^{C}: R_{T} 1.75 min, ee %>99%. The absolute configuration of Example 101 was confirmed to be (S) by re-synthesis using (S)-5-(methylcarbamoyl)-4-(1-phenylethoxy)-1H-pyrrole-2-carboxylic acid (synthesis of which is described in Example 105, Preparation 151) and cyclobutylamine using the method described in Example 78/79, Preparation 117 and subsequent purification. The retention times of this sample, compared with a sample of Example 101, Enantiomer A, were identical as shown by providing a single peak when passed through the column as a mixture with Example 101, Enantiomer A.

Example 102 (Enantiomer B)¹H NMR (400 MHz, MeOH-d₄) δ 7.43-7.27 (m, 5H), 6.46 (s, 1H), 5.38-5.32 (m, 1H), 4.46-4.35 (m, 1H), 2.98 (s, 3H), 2.36-2.26 (m, 2H), 2.12-1.97 (m, 2H), 1.80-1.77 (m, 5H). LCMS^{D}: m/z=340 [M−H]⁻. Chiral HPLC^{C}: R_{T} 2.84 min, ee %>99%. The absolute configuration of Example 102 is believed to be (R) after confirmation that Example 101 is (S) as described above.

### Example 103—N⁵-(4,4-difluorocyclohexyl)-N²-methyl-3-(1-phenylethoxy)-1H-pyrrole-2,5-dicarboxamide, Enantiomer A

### Example 104—N⁵-(4,4-difluorocyclohexyl)-N²-methyl-3-(1-phenylethoxy)-1H-pyrrole-2,5-dicarboxamide, Enantiomer B

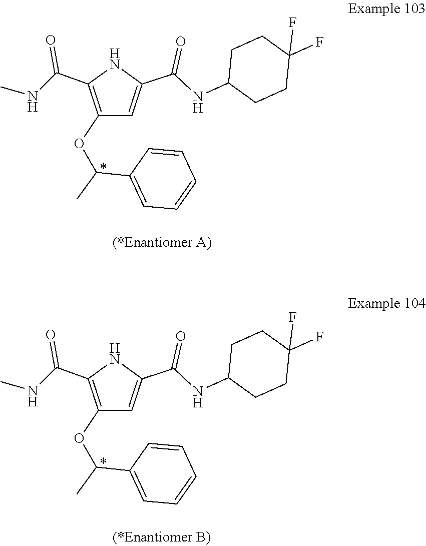 [SMILES: CNC(=O)c1[nH]c(C(=O)NC2CCC(F)(F)CC2)cc1OC(C)c1ccccc1 ; CNC(=O)c1[nH]c(C(=O)NC2CCC(F)(F)CC2)cc1OC(C)c1ccccc1]

Synthesised according to method described in Example 97 and Example 98 (Preparation 149) using 4,4-difluorocyclohexan-1-amine as starting material. The crude product was purified by Prep-HPLC^{F} and further separated by Chiral-Prep-HPLC^{D} to afford Enantiomer A (R_{T} 17.1 min, Example 103, 64 mg, 23%) as a white solid and Enantiomer B (R_{T} 22.4 min, Example 104, 46 mg, 16%) as a white solid.

Example 103 (Enantiomer A)¹H NMR (400 MHz, MeOH-d₄) δ 7.43-7.26 (m, 5H), 6.47 (s, 1H), 5.37-5.31 (m, 1H), 3.97-3.89 (m, 1H), 2.98 (s, 3H), 2.10-2.07 (m, 2H), 1.95-1.81 (m, 4H), 1.71-1.30 (m, 5H). LCMS^{E}: m/z=406 [M+H]⁺. Chiral HPLC^{D}: R_{T} 2.42 min, ee %>99%. The absolute configuration of Example 103 has not yet been assigned unambiguously but is believed to be the S-enantiomer.

Example 104 (Enantiomer B)¹H NMR (400 MHz, MeOH-d₄) δ 7.43-7.28 (m, 5H), 6.47 (s, 1H), 5.37-5.31 (m, 1H), 3.96-3.90 (m, 1H), 2.98 (s, 3H), 2.10-2.07 (m, 2H), 1.95-1.80 (m, 4H), 1.71-1.30 (m, 5H). LCMS^{E}: m/z=406 [M+H]⁺. Chiral HPLC^{D}: R_{T} 2.99 min, ee %>99%. The absolute configuration of Example 104 has not yet been assigned unambiguously but is believed to be the R-enantiomer.

### Example 105—(S)—N⁵-isopropyl-N²-methyl-3-(1-phenylethoxy)-1H-pyrrole-2,5-dicarboxamide

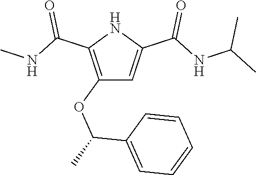 [SMILES: CNC(=O)c1[nH]c(C(=O)NC(C)C)cc1O[C@@H](C)c1ccccc1]

### Preparation 150: Ethyl (S)-5-(methylcarbamoyl)-4-(1-phenylethoxy)-1H-pyrrole-2-carboxylate

Intermediate 3 (8.0 g, 37.7 mmol) and (R)-1-phenylethan-1-ol (9.2 g, 75.4 mmol) were dissolved in THE (80 mL) at room temperature under argon. Triphenyl phosphine (14.8 g, 56.6 mmol) was added to the reaction mixture and allowed to stir at room temperature for 30 min. The resulting solution was cooled at 0° C. and dropwise added diethyl azodicarboxylate (9.8 g, 56.6 mmol). The resulting suspension was allowed to stir at room temperature for 2 h. The reaction mixture was slowly added to water (200 mL) and extracted with ethyl acetate (2×200 mL). The organic layer was washed with brine (2×150 mL) and dried over anhydrous sodium sulphate and concentrated under vacuum to afford crude material. The crude material was purified by normal phase chromatography column chromatography, eluting with (30:70) ethyl acetate/hexane. Solvent evaporation afforded ethyl (S)-5-(methylcarbamoyl)-4-(1-phenylethoxy)-1H-pyrrole-2-carboxylate as a yellow gummy solid (7.0 g, 58%). ¹H NMR: (400 MHz, DMSO) δ 11.60 (s, 1H), 8.99 (s, 1H), 7.46-7.44 (m, 2H), 7.37-7.25 (m, 4H), 5.46-5.41 (m, 1H), 4.16-4.14 (m, 2H), 4.06-4.01 (m, 3H), 2.86 (d, J=3.6 Hz, 3H), 1.59 (d, J=1.8 Hz, 3H).

### Preparation 151: (S)-5-(methylcarbamoyl)-4-(1-phenylethoxy)-1H-pyrrole-2-carboxylic acid Intermediate 4

Ethyl (S)-5-(methylcarbamoyl)-4-(1-phenylethoxy)-1H-pyrrole-2-carboxylate (6.5 g, 20.6 mmol) was dissolved in Ethanol (260 mL) and water (65 mL) at room temperature. Sodium hydroxide (3.2 g, 82.3 mmol) was added to the reaction mixture at room temperature and allowed to stir at 60° C. for 5 h. The resulting solution was cooled to room temperature and concentrated under vacuum distillation. The resulting residue was diluted with water (250 mL) and extracted with ethyl acetate (2×200 mL). The aqueous layer was acidified using 1 N HCL solution to pH 2. The acidic phase was extracted with ethyl acetate (2×250 mL). Fractions were combined and concentrated under vacuum to afford (S)-5-(methylcarbamoyl)-4-(1-phenylethoxy)-1H-pyrrole-2-carboxylic acid, Intermediate 4, as a pink solid. (3.0 g, 50%). ¹H NMR: (400 MHz, DMSO) b 12.58 (br s, 1H), 11.30 (s, 1H), 7.45 (d, J=7.2 Hz, 2H), 7.37-7.32 (m, 3H), 7.13 (s, 1H), 6.35 (s, 1H), 5.42-5.38 (m, 1H), 2.84 (d, J=4.8 Hz, 3H), 1.59 (d, J=6.0 Hz, 3H).

### Preparation 152: (S)—N⁵-isopropyl-NV-methyl-3-(1-phenylethoxy)-1H-pyrrole-2,5-dicarboxamide

Intermediate 4 (0.15 g, 0.52 mmol) was dissolved in DMF (3.8 mL) under nitrogen at room temperature. 1,1′-Carbonyldiimidazole (0.16 g, 1.04 mmol) was added to the reaction mixture and allowed to stir at room temperature for 30 min. Isopropyl amine (0.061 g, 1.04 mmol) was added to the reaction mixture at room temperature The resulting mixture was stirred at room temperature for 2 h. The reaction mixture was quenched with water (25 mL) and extracted with ethyl acetate (2×25 mL). The organic layer was washed with brine solution (2×25 mL) and concentrated under vacuum to afford crude material. The crude material was purified reverse phase flash chromatography (C18 silica 50 μm, eluting with 60:40 acetonitrile/water) to afford (S)—N⁵-isopropyl-/-methyl-3-(1-phenylethoxy)-1H-pyrrole-2,5-dicarboxamide as a white solid (0.035 g, 20%). ¹H NMR: (400 MHz, DMSO) δ 11.31 (s, 1H), 8.07 (d, J=7.2 Hz, 1H), 7.44 (d, J=7.2 Hz, 2H), 7.35 (apparent t, J=7.2 Hz, 2H), 7.28-7.20 (m, 2H), 6.32 (s, 1H), 5.40-5.35 (m, 1H), 3.96-3.88 (m, 1H), 2.86 (d, J=4.8 Hz, 3H), 1.60 (d, J=6.4 Hz, 3H), 1.09 (t, J=6.8 Hz, 6H). LCMS¹: m/z=328 [M−H]⁻.

### Example 106—(S)—N⁵-cyclopropyl-N²-methyl-3-(1-phenylethoxy)-1H-pyrrole-2,5-dicarboxamide

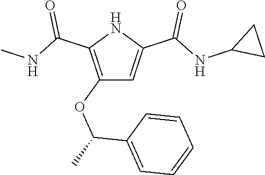 [SMILES: CNC(=O)c1[nH]c(C(=O)NC2CC2)cc1O[C@@H](C)c1ccccc1]

Synthesised according to method described in Example 105 (Preparation 152) using cyclopropyl amine. Afforded title compound as an off-white solid (0.055 g, 32%). ¹H NMR: (400 MHz, DMSO) δ 11.19 (s, 1H), 8.24 (d, J=3.2 Hz, 1H), 7.43 (d, J=7.2 Hz, 2H), 7.35 (apparent t, J=7.6 Hz, 2H), 7.28-7.21 (m 2H), 6.33 (s, 1H), 5.40-5.35 (m, 1H), 2.85 (d, J=4.4 Hz, 3H), 2.72-2.68 (m, 1H) 1.59 (d, J=6.4 Hz, 3H), 0.65-0.62 (m, 2H), 0.43 (t, J=2 Hz, 2H). LCMS¹: m/z=326 [M−H]⁻.

### Example 107—(S)—N²-methyl-N⁵-(oxetan-3-yl)-3-(1-phenylethoxy)-1H-pyrrole-2,5-dicarboxamide

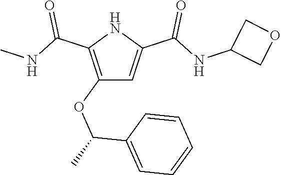 [SMILES: CNC(=O)c1[nH]c(C(=O)NC2COC2)cc1O[C@@H](C)c1ccccc1]

Synthesised according to method described in Example 105 (Preparation 152) using oxetan-3-amine. Afforded title compound as an off-white solid (0.090 g, 50%). ¹H NMR: (400 MHz, DMSO) δ 11.36 (s, 1H), 8.90 (d, J=6 Hz, 1H), 7.44 (d, J=7.2 Hz, 2H), 7.35 (apparent t, J=7.6 Hz, 2H), 7.28-7.24 (m, 2H), 6.37 (s, 1H), 5.41-5.36 (m, 1H), 4.89-4.84 (m, 1H), 4.76-4.71 (m, 2H), 4.47-4.42 (m, 2H), 2.87 (d, J=4.8 Hz, 3H), 1.60 (d, J=6.4 Hz, 3H). LCMS¹: m/z=342 [M−H]⁻.

### Example 108—N⁵-(trans-3-hydroxycyclobutyl)-N²-methyl-3-((S)-1-phenylethoxy)-1H-pyrrole-2,5-dicarboxamide

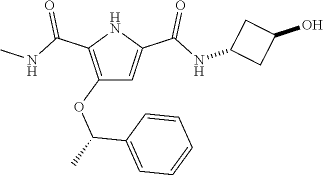

Intermediate 4 (0.15 g, 0.52 mmol) was dissolved in DMF (3.8 mL) under nitrogen at room temperature. 1,1′-Carbonyldiimidazole (0.16 g, 1.04 mmol) was added to the reaction mixture and allowed to stir at room temperature for 30 min. trans-3-aminocyclobutan-1-ol hydrochloride (CAS #1036260-45-3) (0.12 g, 1.04 mmol) and diisopropyl amine (0.074 g, 0.57 mmol) were added to the reaction mixture at room temperature The resulting mixture was stirred at room temperature for 16 h. The reaction mixture was quenched with water (25 mL) and extracted with ethyl acetate (2×25 mL). The organic layer was washed with brine solution (2×25 mL) and concentrated under vacuum to afford crude material. The crude material was purified reverse phase flash chromatography (C18 silica 50 μm, eluting with 60:40 acetonitrile/water) to afford N⁵-((1 r,3S)-3-hydroxycyclobutyl)-N⁵-methyl-3-((S)-1-phenylethoxy)-1H-pyrrole-2,5-dicarboxamide as an off-white solid (0.097 g, 52%). ¹H NMR: (400 MHz, DMSO) δ 11.32 (s, 1H), 8.40 (d, J=6.4 Hz, 1H), 7.43 (d, J=7.2 Hz, 2H), 7.35 (apparent t, J=7.2 Hz, 2H), 7.28-7.21 (m, 2H), 6.31 (s, 1H), 5.40-5.36 (m, 1H), 5.06 (d, J=5.2 Hz, 1H), 4.30-4.20 (m, 2H), 2.87 (d, J=4.8 Hz, 3H), 2.12-2.09 (m, 4H), 1.60 (d, J=6.4 Hz, 3H). LCMS¹: m/z=356 [M−H]⁻.

### Example 109—(S)—N⁵-(3,3-difluorocyclobutyl)-N²-methyl-3-(1-phenylethoxy)-1H-pyrrole-2,5-dicarboxamide

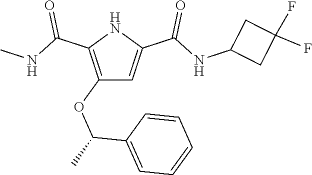 [SMILES: CNC(=O)c1[nH]c(C(=O)NC2CC(F)(F)C2)cc1O[C@@H](C)c1ccccc1]

Synthesised according to method described in Example 108 using 3,3-difluorocyclobutan-1-amine hydrochloride. Afforded the title compound as an off-white solid (0.050 g, 25%). ¹H NMR: (400 MHz, DMSO) δ 11.30 (s, 1H), 8.61 (d, J=6 Hz, 1H), 7.44 (d, J=7.2 Hz, 2H), 7.35 (apparent t, J=7.6 Hz, 2H), 7.28-7.24 (m, 2H), 6.35 (s, 1H), 5.41-5.36 (m, 1H), 4.13 (t, J=6.8 Hz, 1H), 2.98-2.86 (m, 5H), 2.73-2.61 (m, 2H), 1.60 (d, J=6.4 Hz, 3H). LCMS¹: m/z=376 [M−H]⁻.

### Example 110—N⁵—((S)-3,3-difluorocyclopentyl)-N²-methyl-3-((S)-1-phenylethoxy)-1H-pyrrole-2,5-dicarboxamide

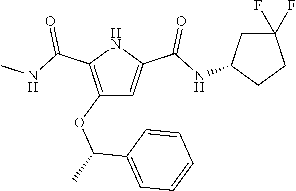 [SMILES: CNC(=O)c1[nH]c(C(=O)N[C@H]2CCC(F)(F)C2)cc1O[C@@H](C)c1ccccc1]

Synthesised according to method described in Example 108 using (S)-3,3-difluorocyclopentan-1-amine hydrochloride to afford the title compound as an off-white solid (0.105 g, 52%). ¹H NMR: (400 MHz, DMSO) δ 11.37 (s, 1H), 8.36 (d, J=6.8 Hz, 1H), 7.44 (d, J=7.2 Hz, 2H), 7.35 (apparent t, J=7.2 Hz, 2H), 7.28-7.23 (m, 2H), 6.34 (s, 1H), 5.41-5.36 (m, 1H), 4.31-4.25 (m, 1H), 2.89-2.86 (m, 3H), 2.46-1.94 (m, 5H), 1.71-1.56 (m, 4H). LCMS¹: m/z=390 [M−H]⁻.

### Example 111—N⁵—((R)-3,3-difluorocyclopentyl)-N²-methyl-3-((S)-1-phenylethoxy)-1H-pyrrole-2,5-dicarboxamide

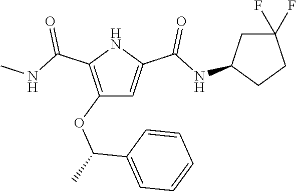 [SMILES: CNC(=O)c1[nH]c(C(=O)N[C@@H]2CCC(F)(F)C2)cc1O[C@@H](C)c1ccccc1]

Synthesised according to method described in Example 108 using (R)-3,3-difluorocyclopentan-1-amine hydrochloride to afford the title compound as an off-white solid (0.048 g, 24%). ¹H NMR: (400 MHz, DMSO) δ 11.35 (s, 1H), 8.35 (d, J=6.8 Hz, 1H), 7.44 (d, J=7.2 Hz, 2H), 7.35 (apparent t, J=7.2 Hz, 2H), 7.28-7.22 (m, 2H), 6.34 (s, 1H), 5.41-5.36 (m, 1H), 4.31-4.25 (m, 1H), 2.87 (d, J=4.4 Hz, 3H), 2.43-1.90 (m, 5H), 1.72-1.67 (m, 1H), 1.60 (d, J=6.4 Hz, 3H). LCMS¹: m/z=390 [M−H]⁻.

### Example 112—(S)—N²-methyl-3-(1-phenylethoxy)-N⁵-(3,3,3-trifluoropropyl)-1H-pyrrole-2,5-dicarboxamide

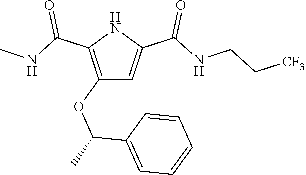 [SMILES: CNC(=O)c1[nH]c(C(=O)NCCC(F)(F)F)cc1O[C@@H](C)c1ccccc1]

Synthesised according to method described in Example 108 using 3,3,3-trifluoropropan-1-amine hydrochloride to afford the title compound as an off-white solid (0.033 g, 17%). ¹H NMR: (400 MHz, DMSO) δ 11.27 (s, 1H), 8.45 (apparent t, J=5.2 Hz, 1H), 7.44 (d, J=7.2 Hz, 2H), 7.35 (apparent t, J=7.6 Hz, 2H), 7.28-7.22 (m, 2H), 6.35 (s, 1H), 5.40-5.35 (m, 1H), 3.42-3.39 (m, 2H), 2.86 (d, J=4.8 Hz, 3H), 2.47-2.42 (m, 2H), 1.60 (d, J=6.4 Hz, 3H). LCMS¹: m/z=382 [M−H]⁻.

### Example 113—N²-methyl-3-((S)-1-phenylethoxy)-N⁵—((S)-tetrahydrofuran-3-yl)-1H-pyrrole-2,5-dicarboxamide

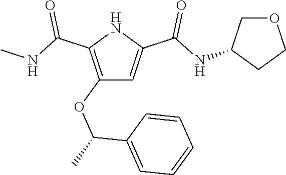 [SMILES: CNC(=O)c1[nH]c(C(=O)N[C@H]2CCOC2)cc1O[C@@H](C)c1ccccc1]

Synthesised according to method described in Example 105 (Preparation 152) using (S)-tetrahydrofuran-3-amine to afford the title compound as an off-white solid (0.050 g, 27%). ¹H NMR: (400 MHz, DMSO) δ 11.41 (s, 1H), 8.38 (d, J=6 Hz, 1H), 7.44 (d, J=7.2 Hz, 2H), 7.35 (apparent t, J=7.6 Hz, 2H), 7.28-7.24 (m, 2H), 6.34 (s, 1H), 5.41-5.36 (m, 1H), 4.35-4.31 (m, 1H), 3.82-3.68 (m, 3H), 3.53-3.50 (m, 1H), 2.87 (d, J=4.8 Hz, 3H), 2.13-2.05 (m, 1H), 1.79-1.72 (m, 1H), 1.60 (d, J=6.4 Hz, 3H). LCMS¹: m/z=356 [M−H]⁻.

### Example 114—N²-methyl-3-((S)-1-phenylethoxy)-N⁵—((R)-tetrahydrofuran-3-yl)-1H-pyrrole-2,5-dicarboxamide

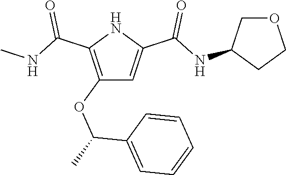 [SMILES: CNC(=O)c1[nH]c(C(=O)N[C@@H]2CCOC2)cc1O[C@@H](C)c1ccccc1]

Synthesised according to method described in Example 105 (Preparation 152) using (R)-tetrahydrofuran-3-amine to afford the title compound as an off-white solid (0.065 g, 35%).

¹H NMR: (400 MHz, DMSO) δ 11.39 (s, 1H), 8.38 (d, J=6 Hz, 1H), 7.44 (d, J=7.6 Hz, 2H), 7.35 (apparent t, J=7.6 Hz, 2H), 7.28-7.21 (m, 2H), 6.35 (s, 1H), 5.41-5.36 (m, 1H), 4.33 (br s, 1H), 3.83-3.66 (m, 3H), 3.50-3.47 (m, 1H), 2.87 (d, J=4.8 Hz, 3H), 2.15-2.06 (m, 1H), 1.81-1.74 (m, 1H), 1.61 (s, 3H). LCMS¹: m/z=356 [M−H]⁻.

### Example 115—N⁵-((1S,3S)-3-hydroxycyclopentyl)-N²-methyl-3-((S)-1-phenylethoxy)-1H-pyrrole-2,5-dicarboxamide

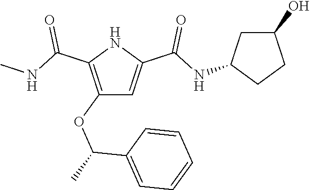 [SMILES: CNC(=O)c1[nH]c(C(=O)N[C@H]2CC[C@H](O)C2)cc1O[C@@H](C)c1ccccc1]

Synthesised according to method described in Example 108 using (1S,3S)-3-aminocyclopentan-1-ol hydrochloride (CAS #1523530-42-8) to afford the title compound as a light yellow solid (0.075 g, 39%). ¹H NMR: (400 MHz, DMSO) δ 11.30 (s, 1H), 8.11 (d, J=7.2 Hz, 1H), 7.44-7.13 (m, 6H), 6.31 (s, 1H), 5.40-5.36 (m, 1H), 4.53 (d, J=3.6 Hz, 1H), 4.29-4.24 (m, 1H), 4.18 (d, J=3.2 Hz, 1H), 2.01-1.78 (m, 3H), 1.60-1.54 (m, 7H), 1.44-1.31 (m, 2H). LCMS¹: m/z=370 [M−H]⁻.

### Example 116 N⁵-(trans-3-hydroxycyclopentyl)-N²-methyl-3-((S)-1-phenylethoxy)-1H-pyrrole-2,5-dicarboxamide

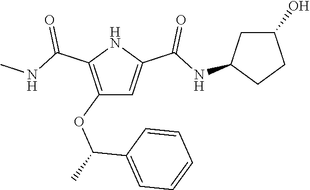 [SMILES: CNC(=O)c1[nH]c(C(=O)N[C@@H]2CC[C@@H](O)C2)cc1O[C@@H](C)c1ccccc1]

Synthesised according to method described in Example 108 using racemic trans-3-aminocyclopentanol hydrochloride (CAS #124555-33-5) to afford the title compound as an off-white solid (0.060 g, 31%). ¹H NMR: (400 MHz, DMSO) δ: 11.30 (s, 1H), 8.11 (d, J=7.2 Hz, 1H), 7.43 (d, J=4.4 Hz, 2H), 7.33 (apparent t, J=7.6 Hz, 2H), 7.28-7.19 (m, 2H), 6.31 (s, 1H), 5.38 (q, J=6.0 Hz, 1H), 4.53 (d, J=2.4 Hz, 1H), 4.29-4.18 (m, 2H), 2.86 (d, J=4.8 Hz, 3H), 2.01-1.96 (m, 1H), 1.88-1.77 (m, 2H), 1.60-1.52 (m, 4H), 1.43-1.41 (m, 2H). LCMS¹: m/z=370 [M−H]⁻.

### Example 117—(S)—N⁵-(bicyclo [1.1.1]pentan-1-yl)-N²-methyl-3-(1-phenylethoxy)-1H-pyrrole-2,5-dicarboxamide

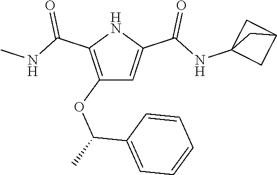 [SMILES: CNC(=O)c1[nH]c(C(=O)NC23CC(C2)C3)cc1O[C@@H](C)c1ccccc1]

Synthesised according to method described in Example 108 using bicyclo [1.1.1]pentan-1-amine hydrochloride to afford the title compound as an off-white solid (0.10 g, 54%). ¹H NMR: (400 MHz, DMSO) δ: 11.16 (s, 1H), 8.72 (s, 1H), 7.43 (d, J=4.4 Hz, 2H), 7.34 (t, J=7.6 Hz, 2H), 7.28-7.20 (m, 2H), 6.31 (s, 1H), 5.38 (q, J=6 Hz, 1H), 2.85 (d, J=4.8 Hz, 3H), 2.40 (s, 1H), 2.00 (s, 6H), 1.61 (t, J=13.2 Hz, 3H). LCMS¹: m/z=352 [M−H]⁻.

### Example 118—(S)—N⁵-(3-hydroxybicyclo [1.1.1]pentan-1-yl)-N²-methyl-3-(1-phenylethoxy)-1H-pyrrole-2,5-dicarboxamide

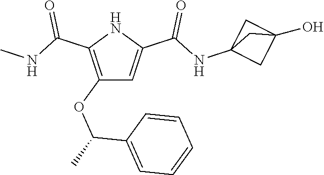 [SMILES: CNC(=O)c1[nH]c(C(=O)NC23CC(O)(C2)C3)cc1O[C@@H](C)c1ccccc1]

Synthesised according to method described in Example 108 using 3-aminobicyclo [1.1.1]pentan-1-ol hydrochloride to afford the title compound as a light yellow solid (0.055 g, 28%). ¹H NMR (400 MHz, CDCl₃) δ 10.06 (s, 1H), 7.41-7.30 (m, 6H), 7.02 (d, J=5.2 Hz, 1H), 6.58 (s, 1H), 6.02 (d, J=3.2 Hz, 1H), 5.22 (m, 1H), 3.01 (d, J=4.8 Hz, 3H), 2.29 (s, 6H), 1.70 (d, J=6.8 Hz, 3H). LCMS¹: m/z=368 [M−H]⁻.

### Example 119—N²-methyl-3-((S)-1-phenylethoxy)-N⁵—((R)-1,1,1-trifluoropropan-2-yl)-1H-pyrrole-2,5-dicarboxamide

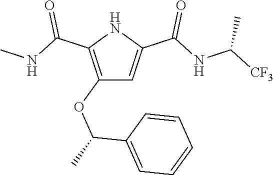 [SMILES: CNC(=O)c1[nH]c(C(=O)N[C@H](C)C(F)(F)F)cc1O[C@@H](C)c1ccccc1]

Synthesised according to method described in Example 108 using (R)-1,1,1-trifluoropropan-2-amine hydrochloride (CAS #177469-12-4) to afford the title compound as a light-yellow solid (0.056 g, 42%). ¹H NMR: (400 MHz, DMSO) δ: 11.50 (s, 1H), 8.61 (d, J=8 Hz, 1H), 7.45 (d, J=7.2 Hz, 2H), 7.35 (apparent t, J=7.6 Hz, 2H), 7.26 (apparent t, J=7.2 Hz, 2H), 6.44 (s, 1H), 5.43-5.38 (m, 1H), 4.74-4.68 (m, 1H), 2.87 (d, J=4.8 Hz, 3H), 1.61 (d, J=6.4 Hz, 3H), 1.27 (d, J=7.2 Hz, 3H). LCMS¹: m/z=384 [M+H]⁺.

### Example 120—N²-methyl-3-((S)-1-phenylethoxy)-N⁵—((S)-1,1,1-trifluoropropan-2-yl)-1H-pyrrole-2,5-dicarboxamide

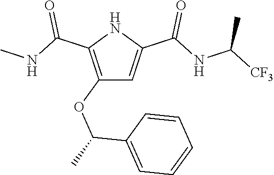 [SMILES: CNC(=O)c1[nH]c(C(=O)N[C@@H](C)C(F)(F)F)cc1O[C@@H](C)c1ccccc1]

Synthesised according to method described in Example 108 using (S)-1,1,1-trifluoropropan-2-amine hydrochloride (CAS #125353-44-8) to afford the title compound as a light-yellow solid (0.068 g, 51%). ¹H NMR: (400 MHz, DMSO) δ: 11.30 (s, 1H), 8.60 (d, J=8 Hz, 1H), 7.45 (d, J=7.2 Hz, 2H), 7.35 (apparent t, J=7.2 Hz, 2H), 7.27 (apparent t, J=6.4 Hz, 2H), 6.44 (s, 1H), 5.40 (s, 1H), 4.72 (d, J=8.4 Hz, 1H), 2.87 (d, J=4 Hz, 3H), 1.60 (d, J=6 Hz, 3H), 1.29 (d, J=6.8 Hz, 3H). LCMS¹: m/z=384 [M+H]⁺.

### Example 121—(S)—N⁵-(tert-butyl)-N²-methyl-3-(1-phenylethoxy)-1H-pyrrole-2,5-dicarboxamide

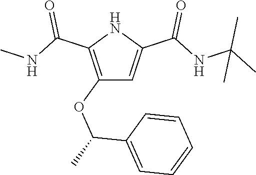 [SMILES: CNC(=O)c1[nH]c(C(=O)NC(C)(C)C)cc1O[C@@H](C)c1ccccc1]

Synthesised according to method described in Example 108 using 2-methylpropan-2-amine to afford title compound as light-yellow solid (0.010 g, 8%). ¹H NMR: (400 MHz, DMSO) δ: 11.30 (br s, 1H), 7.76 (s, 1H), 7.43 (d, J=7.2 Hz, 2H), 7.35 (apparent t, J=7.6 Hz, 2H), 7.19-7.14 (m, 1H), 7.14-7.13 (m, 1H), 6.27 (s, 1H), 5.39-5.36 (m, 1H), 2.86 (d, J=4.8 Hz, 3H), 1.43 (d, J=9.6 Hz, 3H), 1.29 (s, 9H). LCMS¹: m/z=342 [M−H]⁻.

### Example 122—Racemic 3-(1-(4-chlorophenyl) ethoxy)-N⁵-ethyl-N²-methyl-1H-pyrrole-2,5-dicarboxamide

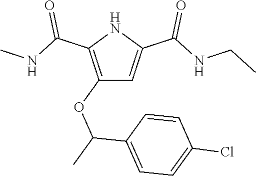 [SMILES: CCNC(=O)c1cc(OC(C)c2ccc(Cl)cc2)c(C(=O)NC)[nH]1]

Intermediate 2 (0.10 g, 0.47 mmol) was dissolved in THE (2 mL) under nitrogen. Potassium tert-butoxide (0.084 g, 0.75 mmol) and 18-crown-6 (0.012 g, 0.047 mmol) were added to the reaction mixture followed by 1-(1-bromoethyl)-4-chlorobenzene (0.20 g, 0.94 mmol) at room temperature. The reaction was stirred at room temperature for 16 h. The resulting suspension was diluted with water (25 mL) and extracted with ethyl acetate (2×25 mL). The organic layer was washed with brine solution (2×25 mL) and concentrated under vacuum to afford crude material. The crude material was purified using reverse phase flash chromatography (C18 silica 50 μm, eluting with (50:50) acetonitrile/water) to afford title compound as a white solid (0.035 g, 21%). ¹H NMR: (400 MHz, DMSO) δ 11.24 (s, 1H), 8.20 (apparent t, J=5.2 Hz, 1H), 7.44 (m, 4H), 7.21 (d, J=4.8 Hz, 1H), 6.32 (s, 1H), 5.39 (d, J=6.4 Hz, 1H), 3.19-3.15 (m, 2H), 2.85 (d, J=4.8 Hz, 3H), 1.60 (d, J=6.4 Hz, 3H) 1.06 (t, J=7.2 Hz, 3H). LCMS¹: m/z=350 [M+H]⁺.

### Example 123—Racemic N⁵-ethyl-3-(1-(3-fluorophenyl) ethoxy)-N²-methyl-1H-pyrrole-2,5-dicarboxamide

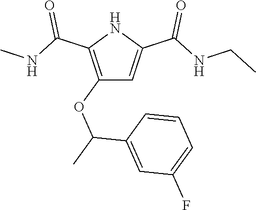 [SMILES: CCNC(=O)c1cc(OC(C)c2cccc(F)c2)c(C(=O)NC)[nH]1]

Synthesised according to method described in Example 122 using 1-(1-bromoethyl)-3-fluorobenzene to afford title compound as an off-white solid (0.007 g, 9%). ¹H NMR: (400 MHz, DMSO) δ 12.26 (s, 1H), 8.25 (apparent t, J=4.8 Hz, 1H), 7.40-7.35 (m, 1H), 7.24-7.17 (m, 2H), 7.09-7.07 (m 1H), 6.79 (d, J=4.8 Hz, 1H), 6.55 (s, 1H), 4.26 (t, J=6.0 Hz, 1H), 3.23-3.20 (m, 2H), 3.08 (t, J=6.4 Hz, 3H), 2.70 (d, J=4.8 Hz, 3H), 1.09 (t, t=7.2 Hz, 3H). LCMS¹: m/z=334 [M+H]⁺.

### Example 124—Racemic N⁵-ethyl-3-(1-(2-fluorophenyl) ethoxy)-N²-methyl-1H-pyrrole-2,5-dicarboxamide

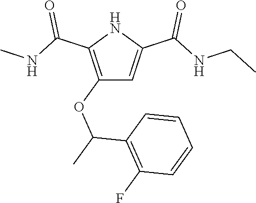 [SMILES: CCNC(=O)c1cc(OC(C)c2ccccc2F)c(C(=O)NC)[nH]1]

Synthesised according to method described in Example 122 using 1-(1-bromoethyl)-2-fluorobenzene to afford title compound as an off-white solid (0.037 g, 23%). ¹H NMR: (400 MHz, DMSO) δ 11.25 (s, 1H), 8.28 (apparent t, J=5.2 Hz, 1H), 7.53 (apparent t, J=7.6 Hz, 1H), 7.36-7.32 (m, 1H), 7.24-7.18 (m, 3H), 6.33 (s, 1H), 5.59-5.55 (m, 1H), 3.20-3.14 (m, 2H), 2.85 (d, J=4.8 Hz, 3H), 1.64 (d, J=6.0, 3H), 1.06 (t, J=7.2 Hz, 3H). LCMS¹: m/z=334 [M+H]⁺.

### Example 125—Racemic N⁵-ethyl-N²-methyl-3-(1-(3-(trifluoromethyl) phenyl) ethoxy)-1H-pyrrole-2,5-dicarboxamide

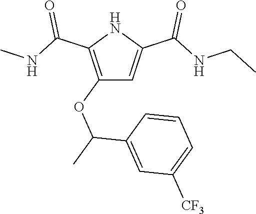 [SMILES: CCNC(=O)c1cc(OC(C)c2cccc(C(F)(F)F)c2)c(C(=O)NC)[nH]1]

Synthesised according to method described in Example 122 using 1-(1-bromoethyl)-3-(trifluoromethyl) benzene to afford title compound as an off-white solid (0.035 g, 19%). ¹H NMR: (400 MHz, DMSO) δ 11.27 (s, 1H), 8.21 (apparent t, J=5.2 Hz, 1H), 7.86 (s, 1H), 7.77 (d, J=7.6 Hz, 1H), 7.65-7.57 (m, 2H), 7.28 (d, J=4.4 Hz, 1H), 6.38 (s, 1H), 5.54-5.49 (m, 1H), 3.29-3.14 (m, 2H), 2.86 (d, J=4.8 Hz, 3H), 1.62 (t, J=6.4 Hz, 3H), 1.07 (t, J=10.8 Hz, 3H). LCMS¹: m/z=382 [M−H]⁻.

### Example 126—Racemic N⁵-ethyl-N²-methyl-3-(1-phenylpropoxy)-1H-pyrrole-2,5-dicarboxamide

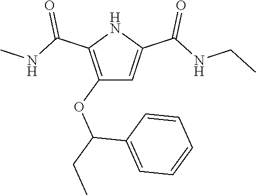 [SMILES: CCNC(=O)c1cc(OC(CC)c2ccccc2)c(C(=O)NC)[nH]1]

Synthesised according to method described in Example 122 using (1-bromopropyl) benzene to afford title compound as an off-white solid (0.02 g, 13%). ¹H NMR: (400 MHz, DMSO) δ 11.20 (s, 1H), 8.20 (apparent t, J=4.8 Hz, 1H), 7.42-7.41 (m, 2H), 7.34 (apparent t, J=7.6 Hz, 2H), 7.28-7.24 (m, 1H), 7.21-7.20 (m, 1H), 6.30 (s, 1H), 5.11 (t, J=6.4, 1H), 3.29-3.11 (m, 2H), 2.87-2.86 (m, 3H), 2.08-2.01 (m, 1H), 1.86-1.79 (m, 1H), 1.05 (t, J=7.2 Hz, 3H) 0.91 (t, J=7.2 Hz, 3H). LCMS¹: m/z=328 [M−H]⁻.

### Example 127—Racemic N⁵-ethyl-3-(1-(4-fluorophenyl) ethoxy)-N²-methyl-1H-pyrrole-2,5-dicarboxamide

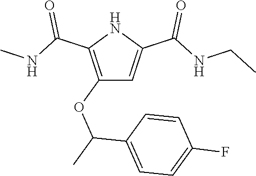 [SMILES: CCNC(=O)c1cc(OC(C)c2ccc(F)cc2)c(C(=O)NC)[nH]1]

Synthesised according to method described in Example 122 using 1-(1-bromoethyl)-4-fluorobenzene to afford title compound as an off-white solid (0.035 g, 21%). ¹H NMR: (400 MHz, DMSO) δ 11.21 (s, 1H), 8.21 (apparent t, J=4.8 Hz, 1H), 7.51-7.41 (m, 2H), 7.21-7.15 (m, 3H), 6.34 (s, 1H), 5.41-5.36 (m, 1H), 3.20-3.14 (m, 2H), 2.85 (d, J=4.8 Hz, 3H), 1.59 (d, J=6.4 Hz, 3H) 1.06 (t, J=7.2 Hz, 3H). LCMS¹: m/z=332 [M−H]⁻.

### Example 128—(S)-5-Acetamido-3-(1-(2-fluorophenyl)ethoxy)-N-methyl-1H-pyrrole-2-carboxamide

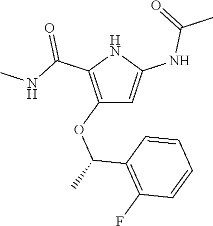 [SMILES: CNC(=O)c1[nH]c(NC(C)=O)cc1O[C@@H](C)c1ccccc1F]

### Preparation 153: ethyl (S)-4-(1-(2-fluorophenyl) ethoxy)-5-(methylcarbamoyl)-1H-pyrrole-2-carboxylate

Ethyl 4-hydroxy-5-(methylcarbamoyl)-1H-pyrrole-2-carboxylate (1.0 g, 4.71 mmol) and (R)-1-(2-fluorophenyl) ethan-1-ol (3 g, 14.15 mmol) were dissolved in THE (60 mL, 20V) at room temperature under argon. Triphenylphosphine (5.56 g, 21.22 mmol) was added to the reaction mixture and allowed to stir at room temperature for 30 min. The resulting solution was cooled at 0° C. and diethyl azodicarboxylate (3.69 g, 21.22 mmol) was added drop wise. The resulting suspension was allowed to stir at room temperature for 3 h. TLC (7:3 Ethyl acetate:Hexane) showed no SM remaining. The reaction mixture was slowly added to water (200 mL) and extracted with ethyl acetate (2×200 mL). The organic layer was washed with brine (2×200 mL) and dried over anhydrous sodium sulphate and concentrated under vacuum to afford crude material. The crude material was purified by normal phase chromatography column chromatography, eluting with (30:70) ethyl acetate/hexane. Solvent reduction gave yellow gummy solid (3.8 g, 80%). LCMS¹ m/z=333 [M−1]⁻

### Preparation 154: ethyl (S)-4-(1-(2-fluorophenyl) ethoxy)-5-(methylcarbamoyl)-1-((2-(trimethylsilyl) ethoxy) methyl)-1H-pyrrole-2-carboxylate

Ethyl (S)-4-(1-(2-fluorophenyl) ethoxy)-5-(methylcarbamoyl)-1H-pyrrole-2-carboxylate (3.7 g, 11.07 mmol) was dissolved in DMF (55.8 mL). The solution was cooled to 0° C. then sodium hydride (0.39 g, 16.61 mmol) was added. After stirring reaction mixture for 30 min at 0° C. 2-(Trimethylsilyl) ethoxymethyl chloride (3.13 g, 18.83 mmol) was added. The reaction mixture was allowed to stir at 0° C. to room temperature for 2 h. TLC (5:5 Ethyl acetate:Hexane) showed no SM remaining. The mixture was quenched using water (200 mL) and extracted using Ethyl acetate (2×200 mL). The organic phase was dried over sodium sulphate and concentrated under vacuum reduced pressure to afford crude material. The crude material was purified by normal phase chromatography column chromatography, eluting with (3:97) ethyl acetate/hexane. Solvent reduction gave brown gummy solid (2.9 g, 56%). LCMS¹ m/z=465 [M+H]⁺

### Preparation 155: (S)-4-(1-(2-fluorophenyl) ethoxy)-5-(methylcarbamoyl)-1-((2-(trimethylsilyl) ethoxy) methyl)-1H-pyrrole-2-carboxylic acid

Ethyl (S)-4-(1-(2-fluorophenyl) ethoxy)-5-(methylcarbamoyl)-1H-pyrrole-2-carboxylate (2.9 g, 6.25 mmol) was dissolved in Methanol (58 mL, 20V) and water (29 mL, 10V) at room temperature. Lithium hydroxide (1.31 g, 31.25 mmol) was added to the reaction mixture at room temperature and allowed to stir at room temperature for 16 h. TLC (5:5 Ethyl acetate:Hexane) showed no SM remaining. The resulting solution was concentrated under vacuum distillation. The resulting residue was diluted with water (100 mL) acidified using 1 N HCL solution till PH became 2. The acidic phase was extracted with ethyl acetate (2×150 mL). Fractions were combined and concentrated under vacuum to afford brown gummy solid. (2.0 g, 73%). LCMS¹ m/z=437 [M+H]⁺

### Preparation 156: tert-butyl (S)-(4-(1-(2-fluorophenyl) ethoxy)-5-(methylcarbamoyl)-1-((2-(trimethylsilyl) ethoxy) methyl)-1H-pyrrol-2-yl) carbamate

(S)-4-(1-(2-fluorophenyl) ethoxy)-5-(methylcarbamoyl)-1-((2-(trimethylsilyl) ethoxy) methyl)-1H-pyrrole-2-carboxylic acid (1.6 g, 3.80 mmol) was dissolved in tert-Butanol (16.6 mL) at room temperature. Diphenyl phosphoryl azide (1.15 g, 4.18 mmol) was added followed by triethylamine (0.57 g, 5.71 mmol) to the reaction mixture. The reaction mixture was heated at 80° C. for 16 h. TLC (5:5 Ethyl acetate:Hexane) showed no SM remaining. The reaction mixture was slowly added to water (100 mL) and extracted with ethyl acetate (100 mL). The organic layer was washed with brine (100 mL) and dried over anhydrous sodium sulphate and concentrated under vacuum to afford crude material. The crude material was purified by normal phase chromatography column chromatography, eluting with (10:90) ethyl acetate/hexane. Solvent reduction gave brown gummy solid (0.7 g, 37%). LCMS¹ m/z=508 [M+H]⁺

### Preparation 155: (S)-5-amino-3-(1-(2-fluorophenyl) ethoxy)-N-methyl-1H-pyrrole-2-carboxamide

Tert-butyl (S)-(4-(1-(2-fluorophenyl) ethoxy)-5-(methylcarbamoyl)-1-((2-(trimethylsilyl) ethoxy) methyl)-1H-pyrrol-2-yl) carbamate (0.58 g, 1.14 mmol) was dissolved in Dioxane (5.8 mL). 4 M HCl in dioxane (1.74 mL) was added. The reaction mixture was allowed to stir at room temperature for 48 h. TLC (5:5 Ethyl acetate:Hexane) showed no SM remaining. The mixture was concentrated under vacuum reduced pressure to afford crude material (0.33 g, crude). The crude material directly used in the next step without further purification. LCMS¹ m/z=278 [M+H]⁺

### Preparation 157: (S)-5-acetamido-3-(1-(2-fluorophenyl) ethoxy)-N-methyl-1H-pyrrole-2-carboxamide

(S)-5-amino-3-(1-(2-fluorophenyl) ethoxy)-N-methyl-1H-pyrrole-2-carboxamide (0.30 g, 1.08 mmol) was dissolved in DCM (3 mL). Triethylamine (0.43 g, 4.33 mmol) was added followed by acetyl chloride (0.085 g, 1.08 mmol) was added. The reaction mixture was allowed to stir at room temperature for 2 h. TLC (9:1 DCM:Methanol) showed no SM remaining. The mixture was concentrated under vacuum reduced pressure to afford crude material. The residue was purified by prep HPLC purification using Instrument: MDAP-01 WATERS 2545 BINARY PUMP WITH WATERS 2489 UV DETECTOR WITH WATERS ACQUITY QDA, Column: Shim-Pack GIST C18 (250 mm×20 mm×5 μm) eluted with a gradient of 0.05% Formic acid in water and acetonitrile. Fractions were combined and lyophilised to give off-white solid (0.005 g, 1%). ¹H NMR: (400 MHz, DMSO) δ 10.40 (s, 1H), 10.16 (s, 1H), 7.52 (t, J=7.6 Hz, 1H), 7.34 (q, J=7.2 Hz, 1H), 7.21 (t, J=8.0 Hz, 2H), 6.82 (d, J=4.8 Hz, 1H), 6.57 (s, 1H), 5.52 (q, J=6.0 Hz, 1H), 2.81 (d, J=4.4 Hz, 3H), 1.96 (s, 3H), 1.63 (d, J=6.4 Hz, 3H). LCMS¹ m/z=320 [M+H]⁺

### Example 129: Biological Activity

### Primary Activity

Exemplary compounds of the disclosure are active against BRD4 BD2 and selective over BRD4 BD1. BRD4 is a representative example of the BET family, as the binding sites of all BET family members are structurally similar. The experimental methods and results (Table 1) are provided hereinafter.

### Bromodomain Assay Procedure

NanoBRET assay was carried out according to the manufacturer's suggested protocol (Promega, Madison, WI). HEK293 cells were transfected using NanoLuc-BRD4-BD1 or NanoLuc-BRD4-BD2 fusion vectors and incubated at 37° C. in an atmosphere of 5% CO₂ for 20-24 hours. The transfected cells were then dispensed into 96-well plates using 90 μl cell suspension per well containing 2×10⁵ cells/mL in Opti-MEM and 1× final concentration of tracer. 90 μL per well of cell suspension without tracer was also dispensed into at least 3 wells as “No tracer control samples” for background correction. Serially diluted test compounds were prepared at 1000× concentration in DMSO and further diluted to 10× concentration in Opti-MEM. 10 μL per well of the serially diluted 10× test compound was added to the 96-well plates containing cells with 1× tracer, to give a final top concentration of test compound of 10 μM. Plates were then incubated at 37° C.+5% CO₂ incubator for 2 hours. Immediately prior to BRET measurements, a 3× solution consisting of 1:166 dilution of Nano-Glo® Substrate plus a 1:500 dilution of Extracellular NanoLuc Inhibitor in Opti-MEM was prepared and 50 μL per well was added to the cells. Donor emission (450 nm) and acceptor emission (610 nm) were measured using PHERAstar (BMG LabTech). For data analysis, the raw BRET ratio was generated and converted to milliBRET units with background correction using the formula: [(Acceptorₛₐₘₚₗₑ/Donorₛₐₘₚₗₑ)−(Acceptor_{no tracer control}/Donor_{no tracer control})]×1000. The mBU data was plotted as a function of compound concentration and IC₅₀s for BRET assay were determined by nonlinear regression analysis of concentration response curves using the Collaborative Drug Discovery Vault software. The fold-selectivity was calculated by dividing the IC₅₀ for BRD4 BD1 by the IC₅₀ for BRD4 BD2.

TABLE 1
IC₅₀s and fold selectivity's of
exemplary compounds of the disclosure

| Example | BRD4 BD1 (IC₅₀) | BRD4 BD2 (IC₅₀) | Fold selectivity |
|---|---|---|---|
| 1 | # | +++ | X |
| 2 | # | ++++ | XX |
| 3 | # | ++++ | XX |
| 4 | # | + | — |
| 5 | # | ++++ | XX |
| 6 | # | +++ | X |
| 7 | # | +++ | X |
| 8 | # | + | — |
| 9 | # | + | — |
| 10 | # | + | — |
| 11 | # | + | — |
| 12 | # | +++ | X |
| 13 | # | +++ | X |
| 14 | # | +++ | X |
| 15 | # | +++ | X |
| 16 | # | +++ | X |
| 17 | # | ++++ | XX |
| 18 | # | +++++ | XXX |
| 19 | # | + | — |
| 20 | # | ++++ | XX |
| 21 | # | + | — |
| 23 | # | +++++ | XXX |
| 23 (2)* | # | + | — |
| 24 | # | ++ | X |
| 24 (2)* | # | + | — |
| 25 | # | +++++ | XXX |
| 26 | # | + | — |
| 27 | # | +++++ | XXX |
| 28 | # | + | — |
| 29 | # | ++++ | XX |
| 31 | # | ++++ | XX |
| 33 | # | ++++ | XX |
| 35 | # | +++++ | XXX |
| 37 | # | ++ | — |
| 38 | # | ++++ | XX |
| 39 | # | ++++ | XX |
| 41 | # | +++ | X |
| 42 | # | + | — |
| 43 | # | ++++ | XX |
| 45 | # | ++++ | XX |
| 47 | # | ++++ | XX |
| 49 | # | +++++ | XXX |
| 50 | # | + | — |
| 51 | # | +++++ | XXX |
| 52 | # | + | — |
| 53 | # | ++++ | XX |
| 54 | # | ++++ | XX |
| 55 | # | + | — |
| 57 | # | +++ | X |
| 58 | # | ++++ | XX |
| 59 | # | ++++ | XX |
| 60 | # | ++++ | XX |
| 61 | # | ++++ | XX |
| 62 | # | +++++ | XXX |
| 63 | # | + | — |
| 64 | # | + | — |
| 65 | # | + | — |
| 66 | # | + | — |
| 67 | # | + | — |
| 68 | # | +++++ | XXX |
| 69 | # | +++++ | XXX |
| 70 | # | +++++ | XXX |
| 72 | # | + | — |
| 73 | # | ++ | X |
| 74 | # | ++ | X |
| 75 | # | ++++ | XX |
| 76 | # | + | — |
| 77 | # | + | — |
| 78 | # | + | — |
| 79 | # | ++ | — |
| 80 | # | +++++ | XXX |
| 81 | # | ++++ | XX |
| 82 | # | + | — |
| 83 | # | +++++ | XXX |
| 84 | # | ++ | X |
| 85 | # | ++ | X |
| 86 | # | ++ | X |
| 87 | # | ++++ | XX |
| 88 | # | ++++ | XX |
| 89 | # | ++++ | XX |
| 90 | # | +++ | X |
| 91 | # | + | — |
| 92 | # | + | — |
| 93 | # | + | — |
| 94 | # | + | — |
| 95 | # | +++ | X |
| 96 | # | +++ | X |
| 96(2)* | # | + | — |
| 97 | # | ++++ | XX |
| 98 | # | + | — |
| 99 | # | ++++ | XX |
| 100 | # | + | — |
| 101 | # | +++++ | XXX |
| 102 | # | + | — |
| 103 | # | ++++ | XX |
| 104 | # | + | — |
| 105 | # | +++ | X |
| 106 | # | ++++ | XX |
| 107 | # | ++++ | XX |
| 108 | # | ++++ | XX |
| 109 | # | +++ | X |
| 110 | # | +++ | X |
| 111 | # | +++ | X |
| 112 | # | +++ | X |
| 113 | # | +++ | X |
| 114 | # | +++ | X |
| 115 | # | ++++ | XX |
| 116 | # | ++++ | XX |
| 117 | # | ++++ | XX |
| 118 | # | ++++ | XX |
| 119 | # | + | — |
| 120 | # | + | — |
| 121 | # | +++ | X |
| 122 | # | ++++ | XX |
| 123 | # | +++ | X |
| 124 | # | ++++ | XX |
| 125 | # | +++ | X |
| 126 | # | +++ | X |
| 127 | # | ++++ | XX |
| 128 | # | ++++ | XX |

Note:
If IC₅₀ of one or both BD2 and BD1 are at the upper limit then selectivity is qualitative.
Key
+ BRD4 BD2 IC₅₀ ≥10 μM
++ BRD4 BD2 IC₅₀ >1 μM and <10 μM
+++ BRD4 BD2 IC₅₀ >0.2 μM and ≤1 μM
++++ BRD4 BD2 IC₅₀ >0.05 μM and ≤0.2 μM
+++++ BRD4 BD2 IC₅₀ ≤0.05 μM
# BRD4 BD1 IC₅₀ ≥10 μM
## BRD4 BD1 IC₅₀ >1 μM and <10 μM
### BRD4 BD1 IC₅₀ >0.5 μM and ≤1 μM
#### BRD4 BD1 IC₅₀ >0.05 μM and ≤0.5 μM
— Fold ≥0 and ≤2
X Fold >2 and ≤50
XX Fold >50 and ≤200
XXX Fold >200

the same test was performed on a second batch of each of examples 23, 24, and 96. Without wishing to be bound by theory, it is believed that the inter-batch variation may be a result of inorganic impurities, undetectable by NMR or LCMS, or sample weighing errors, which may have impacted the molarity of the stock solution or the solubility of the repeat batches. For the absence of doubt, the following table (Table 1A) shows that all batches demonstrated inhibitory activity.

TABLE 1A

| | BRD4 BD2% Activity |
|---|---|
| Example | Remaining @ 10 uM |
| 23 | 22 |
| 23 (2) | 61 |
| 24 | 41 |
| 24 (2) | 78 |
| 96 | 30 |
| 96 (2) | 64 |

### Example 130: Plasma Stability

Exemplary compounds of the disclosure are stable upon incubation in mouse plasma. Stability is expressed as a % remaining after 120 minutes. The experimental methods and results (Table 2) are provided hereinafter.

### Plasma Stability Assay, Method 1

From stock solution of compound in 10 mM DMSO, a working solution of 1 mM is prepared by diluting the compound in DMSO. To 398 μL of plasma pre-warmed at 37° C. for 15 minutes), 2 μL of working solution of compound is added, resulting in a final concentration of 5 μM (0.5% DMSO) for the compound. The sample is then incubated at 37° C. Aliquot is withdrawn at specified time-points (0, 15, 30, 60 and 120 mins). The reaction is stopped by addition of 450 μL cold acetonitrile containing internal standard (IS). The sample are vortexed mixed for minutes then centrifuged at 3,220 g for 30 minutes. Aliquots of the supernatant is analysed using LC-MS/MS. The percent remaining of the compound at each time point is calculated with respect to the control sample (0 min time-point).

### Plasma Stability Assay, Method 2

A working solution of compound (500 μM) is prepared in DMSO (100%). To 735 μL of plasma, 15 μL of working solution of compound is added—resulting in a final concentration of 10 μM (2% DMSO) for the compound. The sample is then incubated at 37° C. Aliquot is withdrawn at time-points—0, 15, 30, 60, 90 and 120 mins. The reaction is stopped by using chilled acetonitrile containing internal standard (IS). The samples are centrifuged and the supernatants analyzed using LC-MS/MS. The percent remaining of the compound at each time point is calculated with respect to the control sample (0 min time-point).

TABLE 2
Plasma stability of exemplary compounds of the disclosure

| | Plasma Stability | | | |
|---|---|---|---|---|
| | (% remaining at 120 minutes) | | | |
| Example | Rat | Mouse | Human | Method |
| 27 | 47 | | | 2 |
| 29 | 89 | | | 2 |
| 31 | 81 | | | 2 |
| 33 | 85 | | | 2 |
| 35 | 67 | | | 2 |
| 39 | 93 | | | 2 |
| 43 | 20 | | | 2 |
| 45 | 88 | | | 2 |
| 47 | 76 | | | 2 |
| 49 | 90 | | | 2 |
| 51 | 93 | | | 2 |
| 53 | 91 | | | 2 |
| 54 | 93 | | | 2 |
| 55 | 88 | | | 2 |
| 59 | 50 | | | 2 |
| 61 | 81 | | | 2 |
| 62 | 82 | | | 2 |
| 68 | 75 | | | 2 |
| 69 | 83 | | | 2 |
| 70 | 92 | | | 2 |
| 72 | 9 | | | 2 |
| 97 | >99 | 93 | >99 | 1 |
| 101 | 61 | >99 | 93 | 1 |

### Example 131: Intrinsic Clearance in Liver Microsomes

BET protein inhibitors with a long half-life in liver microsomes from various species are in some embodiments promising oral drug candidates. Some of the exemplary compounds of the current disclosure have a long half-life in liver microsomes, expressed in minutes. The experimental methods and results (Table 3) are provided hereinafter.

### Intrinsic Clearance in Liver Microsomes Assay, Method 1

From stock solution of compound on 10 mM DMSO, a working solution of 0.1 mM is prepared by diluting the compound in DMSO. 6.25 μL of a 20 mg/mL solution of microsomes in PBS pH 7.4 buffer is added to 216.25 μL PBS pH 7.4 buffer (final microsome concentration—0.5 mg/mL). 25 μL of 10 mM solution of NADPH in PBS pH 7.4 buffer is added to the incubations (final NADPH concentration—1 mM). The mixture is pre-warmed to 37° C. for 10 minutes. 2.5 μL of test compound working solution is added (final compound concentration—1 μM with 1% DMSO). Aliquots of 30 μL are taken from the reaction solution at 0.5, 5, 15, 30 and 60 minutes. The reaction was stopped by the addition of 5 volumes of cold acetonitrile with IS (100 nM alprazolam, 200 nM caffeine and 100 nM tolbutamide). Samples were centrifuged at 3, 220 g for 40 minutes. Aliquots of 100 μL of the supernatant are mixed with 100 μL of ultra-pure H₂O and used for LC-MS/MS analysis. The percent compound remaining at each time point is calculated with respect to that of the 0.5 min sample. The data are analysed to calculate half-life. Control experiments are run without NADPH and blank samples are prepared using DMSO without the test compound.

### Intrinsic Clearance in Liver Microsomes Assay, Method 2

A 50 mM stock solution (in DMSO) is prepared for the compound. From the stock solution, a working solution of 0.5 mM is prepared by diluting the compound in DMSO. This concentration of working solution is prepared considering a final concentration of 1 μM with 0.1% DMSO. The compound (1 μL of working solution) is spiked in PBS with pH 7.4 (22 μL) at a concentration of 1 μM. Subsequently, 110 μL of 10 mM NADPH is added (as a co-factor). The sample is incubated at 37° C. for 15 min. Following this, pre-warmed liver microsomes (27.5 μL; final protein conc. 0.5 mg/mL) are added. The samples are then incubated at 37° C. Aliquots of samples are withdrawn at 0, 5, 15, 30, 45 and 60 min. The reaction is stopped by using chilled acetonitrile containing internal standard. The samples are centrifuged, and the supernatants analysed by LC-MS/MS. The percent compound remaining at each time point is calculated with respect to that of the 0 min sample. The data are then analysed to calculate half-life and intrinsic clearance (CLᵢₙₜ). Control experiments are run without NADPH and blank samples are prepared using DMSO without the test compound.

TABLE 3
Intrinsic clearance of exemplary compounds of the disclosure

| | Half-Life (T_{1/2}) | | |
|---|---|---|---|
| | (min) | | |
| Example | Mouse | Rat | Method |
| 27 | | 47 | 1 |
| 29 | | >60 | 1 |
| 31 | | >60 | 1 |
| 33 | | >60 | 1 |
| 35 | | 46 | 1 |
| 38 | | >60 | 1 |
| 39 | | >60 | 1 |
| 41 | | >60 | 1 |
| 45 | | >60 | 2 |
| 47 | | >60 | 2 |
| 49 | | >60 | 2 |
| 51 | | >60 | 2 |
| 53 | | >60 | 2 |
| 54 | | >60 | 2 |
| 55 | | >60 | 2 |
| 59 | | >60 | 1 |
| 61 | | >60 | 2 |
| 62 | | >60 | 1 |
| 68 | | 23 | 2 |
| 69 | | 21 | 2 |
| 70 | | 60 | 1 |
| 72 | | 13 | 2 |
| 95 | | >60 | 1 |
| 97 | 70 | >60 | 1 |
| 98 | | >60 | 1 |
| 99 | | 39 | 1 |
| 100 | | 19 | 1 |
| 101 | 27 | 58 | 1 |
| 102 | | 41 | 1 |
| 103 | | 19 | 1 |
| 104 | | 10 | 1 |
| 105 | >60 | | 2 |
| 106 | >60 | | 2 |
| 107 | >60 | | 2 |
| 108 | >60 | | 2 |
| 109 | >60 | | 2 |
| 110 | >60 | | 2 |
| 111 | >60 | | 2 |
| 112 | >60 | | 2 |
| 113 | >60 | | 2 |
| 114 | >60 | | 2 |
| 115 | >60 | | 2 |
| 116 | >60 | | 2 |
| 117 | >60 | | 2 |
| 118 | >60 | | 2 |

### Example 132: Thermodynamic Solubility

BET protein inhibitors with a high aqueous solubility are in some embodiments promising oral drug candidates. Some of the exemplary compounds of the current disclosure have high solubility (measured in μM) in Fasted State Simulated Intestinal Fluid (FaSSIF) pH 6.5 buffer. The experimental methods and results (Table 4) are provided hereinafter.

### Thermodynamic Solubility Assay, Method 1

FaSSIF v2 buffer is freshly prepared prior to use according to the suppliers' instructions and the pH adjusted to 6.5 using 1 M NaOH or 1 M HCl as required. From 1 mM working solution of test compound in DMSO, the λₘₐₓ of the test compound is determined by scanning from 200 nm to 700 nm, in 1 nm steps with an EPOCH UV/Visible spectrophotometer (Biotek Instruments Inc.). A 12-point standard curve of compound concentration versus absorption at λₘₐₓ is generated via two-fold serial dilutions of the 1 mM working solution with DMSO. Approximately 1 mg of test compound is accurately weighed into an Eppendorf tube and the appropriate volume of FaSSIF buffer is added to make a nominal concentration of 1 mg/mL. The tube is incubated on a rotator at 20 rpm, 25° C. for 24 hrs. Samples are then filtered using 0.45 μm filters (Sartorious, CAT #1776C) and the filtrate subjected to UV-vis spectroscopy analysis. The absorption at λₘₐₓ is compared to the standard curve to determine the concentration of compound in solution after 24 hours.

### Thermodynamic Solubility Assay, Method 2

FaSSIF buffer was prepared according to the suppliers' instructions and the pH adjusted to 6.5 using 1 M NaOH or 1 M HCl as required. To each vial in a cap-less solubility sample plate, solid test compound was diluted with FaSSIF buffer to a final concentration of 1 mg/mL. A stir stick was added to each vial and vials sealed with a moulded PTDE/SIL 96-well plate cover. The plate was shaken at 25° C. at 1,100 rpm using an Eppendorf Thermomixer Comfort plate shaker. The plates were filtered using a vacuum manifold and diluted with a mixture of H₂O and acetonitrile containing internal standard. The concentration of the test compound in the filtrate was determined by LCMS analysis with reference to the internal standards to determine the solubility in the buffer.

TABLE 4
Thermodynamic solubility in FaSSIF pH 6.5 buffer
of exemplary compounds of the disclosure

| | Thermodynamic Solubility | |
|---|---|---|
| Example | FaSSIF pH 6.5 (μM) | Method |
| 29 | 1230 | 2 |
| 33 | 685 | 2 |
| 59 | 635 | 2 |
| 62 | 320 | 2 |
| 70 | 365 | 2 |
| 97 | 418 | 1 |
| 98 | 429 | 1 |
| 99 | 359 | 1 |
| 100 | 377 | 1 |
| 101 | 193 | 1 |
| 102 | 188 | 1 |
| 103 | 57 | 1 |
| 104 | 47 | 1 |

### Example 133: In Vivo Pharmacokinetics

BET protein inhibitors with a long half-life and good oral bioavailability in in vivo DMPK parameters are promising oral drug candidates. Some of the exemplary compounds of the current disclosure have a long half-life and good oral bioavailability in in vivo DMPK parameters in the Sprague-Dawley rat following intravenous (IV) and oral (PO) dosing. The experimental methods and results (Table 5) are provided hereinafter.

### Sprague-Dawley Rat Pharmacokinetics, Method 1

Compounds were formulated in 5% DMSO, 40% PEG-400, 55% Milli-Q water at a concentration of 0.2 mg/mL for IV and at 0.5 mg/mL for PO. Two male Sprague-Dawley rats per arm were dosed with up to 5 compounds in cassette mode, which each compound being dosed at 1 mg/kg IV and 5 mg/kg PO. Blood was sampled at various timepoints and plasma isolated. 50 μL of plasma with 5 μL of blank Sprague-Dawley rat plasma were added to 200 μL of acetonitrile containing IS mixture. The samples were vortexed for 30 s. After centrifugation at 4 degrees Celsius, 3900 rpm for 15 min, the supernatant was diluted 3 times with water. 20 μL of diluted supernatant was injected into the LC/MS/MS system for quantitative analysis and concentration of analyte determined by comparison with matrix matched standard curve. WinNonlin (Phoenix™, version 8.3) or other similar software may be used for pharmacokinetic calculations.

TABLE 5
Sprague-Dawley rat pharmacokinetic parameters following
IV and PO dosing of exemplary compounds of the disclosure

| | |
|---|---|
| IV | PO |

| | Dose | T_{1/2} | Dose | F | |
|---|---|---|---|---|---|
| Example | (mg/kg) | (hr) | (mg/kg) | (%) | Method |
| 18 | 1 | 1.1 | 5 | 30 | 1 |
| 20 | 1 | 0.4 | 5 | 46 | 1 |
| 27 | 1 | 0.4 | 5 | 47 | 1 |
| 31 | 1 | 0.6 | 5 | 15 | 1 |
| 35 | 1 | 0.5 | 5 | 65 | 1 |
| 38 | 1 | 1.4 | 5 | 66 | 1 |
| 39 | 1 | 0.5 | 5 | 11 | 1 |
| 41 | 1 | 0.3 | 5 | 69 | 1 |
| 43 | 1 | 0.5 | 5 | 58 | 1 |
| 59 | 1 | 1.1 | 5 | 70 | 1 |
| 62 | 1 | 0.5 | 5 | 8 | 1 |
| 70 | 1 | 0.6 | 5 | 44 | 1 |
| 97 | 1 | 1.4 | 5 | 60 | 1 |
| 98 | 1 | 1.2 | 5 | 31 | 1 |
| 101 | 1 | 0.9 | 5 | 66 | 1 |
| 102 | 1 | 2.7 | 5 | 13 | 1 |

### Example 134: Experimental Method A—Preparation of formulations used in Experimental Method B and C

### Compounds and Compound Formulations

TABLE 6A
Compounds and Compound formulations - where Example 101 is provided in a propylene glycol-
based formulation, e.g., for dog pK study 1, for CIA study, and for rat pK study.

| | Dose Level | | Concentration | |
|---|---|---|---|---|
| Compound | (mg/kg) | Preparation | (mg/mL) | Frequency |
| Vehicle | — | 20% propylene glycol/20% | — | RT |
| | | Vitamin E TPGS/60% | | |
| | | water | | |
| Dexamethasone | 0.1 | Saline | 0.02 | Prepared fresh daily |
| GSK620 | 10 | 1% Methylcellulose | 2 | No need to prepare |
| Example 101 | 3 | 20% propylene glycol/20% | 0.3 | fresh daily |
| | | Vitamin E TPGS/60% | | |
| | | water | | |
| Example 101 | 10 | 20% propylene glycol/20% | 1 | |
| | | Vitamin E TPGS/60% | | |
| | | water | | |
| Example 101 | 30 | 20% propylene glycol/20% | 3 | |
| | | Vitamin E TPGS/60% | | |
| | | water | | |

TABLE 6B
Compound formulations where Example 101 is provided in
a HPβCD based formulation, e.g., for dog pK study 2

| | Dose Level | | Concentration |
|---|---|---|---|
| Compound | (mg/kg) | Preparation | (mg/mL) |
| Example 101 | 1 | HPβCD (20% solution w/v/ | 0.5 mg/mL |
| | | DMSO (99/1) | |

The structure of GSK620 is

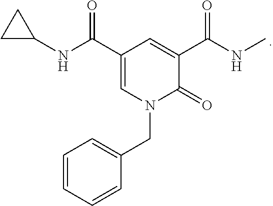 [SMILES: CNC(=O)c1cc(C(=O)NC2CC2)cn(Cc2ccccc2)c1=O]

The structure of Example 101 (*Enantiomer A) is

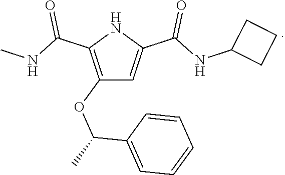 [SMILES: CNC(=O)c1[nH]c(C(=O)NC2CCC2)cc1O[C@@H](C)c1ccccc1]

The structure of Example 23 is

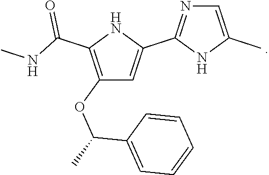

The structure of Example 80 is

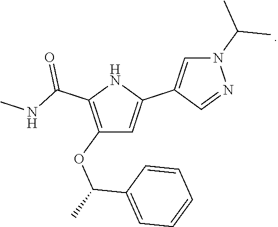 [SMILES: CNC(=O)c1[nH]c(-c2cnn(C(C)C)c2)cc1O[C@@H](C)c1ccccc1]

### Sensitizing Agents

The first sensitizing agent (CFA+Collagen emulsion) was prepared by adding 1.5 mL Completed Freud's Adjuvant to a syringe with a 3-way stopcock and then adding dropwise into 1.5 mL bovine type II collagen (2 mg/mL) while mixing at 30,000 rpm for 2 minutes by a homogenizer. The emulsion was cooled in an ice water bath during the formulation. The emulsion is prepared fresh immediately prior to injection.

The second sensitizing agent (IFA+Collagen emulsion) was prepared by adding 1.5 mL Incomplete Freud's Adjuvant to a syringe with a 3-way stopcock and then adding dropwise into 1.5 mL bovine type II collagen (2 mg/mL) while mixing at 30,000 rpm for 2 minutes by a homogenizer. The emulsion was cooled in an ice water bath during the formulation. The emulsion is prepared fresh immediately prior to injection.

### Example 135: Experimental Method B—Pharmacokinetics of Example 101 in Beagle Dogs

The purpose of this study was to examine the pharmacokinetics of Example 101 in beagle dogs. Two beagle dog studies were undertaken, one where Example 101 was formulated in a propylene glycol-based formulation (PK study 1—PO and IV) and the other where Example 101 was formulated in a HPβCD based formulation (PK study 2—IV). An oral PK study was also undertaken in Lewis rats—see Experimental Method C.

For PK study 1, two solutions of different concentrations of Example 101 were prepared in 20% propylene glycol/20% Vitamin E TPGS/60% water as outlined in Experimental Method A. The Example 101 solutions administered were then administered to animals either intravenously (IV) or orally (PO) and animals were then monitored over a 24 h period. Body weight was recoded prior to dosing, and detailed clinical observations were made prior to dosing and throughout the study, as needed.

The solutions of Example 101 in 20% propylene glycol/20% Vitamin E TPGS/60% water were prepared freshly prior to use. For IV administration, 43.30 mg Example 101 was dissolved in 17.320 mL propylene glycol with the help of a vortex mixer and sonification. Subsequently, 17.320 mL Vitamin E TPGS was added, and the solution was vortexed and sonicated. Then, 51.960 mL water was added, and solution was then heated. For PO administration, 199.47 mg Example 101 was dissolved in 39.894 mL propylene glycol with the help of a vortex mixer and sonification. Subsequently, 39.894 mL Vitamin E TPGS was added, and the solution was vortexed and sonicated. Then, 119.682 mL water was added, and the solution was heated.

TABLE 7A
Experimental treatment groups in PK study 1

| | Number of | Example 101 | | Administration |
|---|---|---|---|---|
| Group | animals | Concentration | Dose | Route |
| 1 | 3 | 0.5 mg/ml | 1 mg/kg | IV |
| 2 | 3 | 1.0 mg/ml | 5 mg/kg | PO |

Blood samples (˜0.3 mL) were collected according to a schedule (detailed below) by venipuncture of peripheral veins except for the dosing vein, and EDTA-K₂ was used as anticoagulant. Within 30 minutes of collection, the samples were centrifuged at approximately 2000× g for 10 minutes at 4° C. to obtain blood plasma. The plasma samples were immediately divided into two aliquots (˜75 μL each), transferred to a cryogenic vial, and stored in a freezer at −75±15° C. prior to analysis.

Blood samples were collected at 0.25, 0.5, 1, 2, 4, 8, and 24 h following the initial dosing (0 h).

The concentration of Example 101 in the blood plasma was determined through liquid chromatography-mass spectrometry (LC-MS). The experiments were conducted on a Shimadzu DGU-20A5R and AB API 5500+LC/MS/MS instrument with an Agilent Poroshell EC-C18 4 μm (50×2.1 mm) column. A gradient of 95% water (0.1% formic acid)/95% acetonitrile (0.1% formic acid) was used as the mobile phase. The sample injection volume was 1 μL, and the flow rate was 0.6 mL/min. The gathered data were used for pharmacokinetic calculations using T½, C0, AUClast, AUCinf, MRTinf, CI, Vss, and Number of Points for Regression as parameters.

For PK study 2, a solution of Example 101 was prepared in HPβCD (20% solution w/v)/DMSO (99/1) with a concentration of 0.5 mg/mL. The Example 101 solution was then administered to animals intravenously (IV), and animals were monitored over a 24 h period. Body weight was recoded prior to dosing, and detailed clinical observations were made prior to dosing and throughout the study, as needed.

The 0.5 mg/mL solution of Example 101 was prepared by dissolving 39.72 mg of Example 101 in 0.794 mL of DMSO with vortex and sonication. 78.646 mL of HPβCD (20% solution w/v) was then added with vortex and sonication.

TABLE 7B
Experimental treatment group in PK study 2

| | Number of | Example 101 | | Administration |
|---|---|---|---|---|
| Group | animals | Concentration | Dose | Route |
| 1 | 3 | 0.5 mg/ml | 1 mg/kg | IV |

Blood samples (˜0.3 mL) were collected according to a schedule (shown below) by venipuncture of peripheral veins except for the dosing vein, and EDTA-K₂ was used as anticoagulant. Within 30 minutes of collection, the samples were centrifuged at approximately 2000× g for 10 minutes at 4° C. to obtain blood plasma. The plasma samples were immediately divided into two aliquots (˜75 μL each), transferred to a cryogenic vial, and stored in a freezer at −75±15° C. prior to analysis.

Blood samples were collected at 0.083, 0.25, 0.5, 1, 2, 4, 8, and 24 h following the initial dosing (0 h).

The concentration of Example 101 in the blood plasma was determined through liquid chromatography-mass spectrometry (LC-MS). The experiments were conducted on a Shimadzu DGU-20A5R and AB API 5500+LC/MS/MS instrument with an Agilent Poroshell EC-C18 4 μm (50×2.1 mm) column. A gradient of 95% water (0.1% formic acid)/95% acetonitrile (0.1% formic acid) was used as the mobile phase. The sample injection volume was 1 μL, and the flow rate was 0.6 mL/min. The gathered data were used for pharmacokinetic calculations using T½, C0, AUClast, AUCinf, MRTinf, CI, Vss, and Number of Points for Regression as parameters.

### Example 136: Experimental Method C—Collagen-Induced Arthritis in Animals Treated with Example 101 Formulations and Vehicle

Collagen-induced arthritis (CIA) is a complex model of autoimmune-mediated arthritis. CIA is induced in rats by immunization with type II collagen and develops joint arthritis pathology that is similar to rheumatoid arthritis. Accordingly, CIA has been used extensively as a valuable model for studying rheumatoid arthritis.

The collagen-induced arthritis model in rats was used to assess the efficacy of oral delivery of Example 101 at three doses (3 mg/kg, 10 mg/kg, and 30 mg/kg). Experimental groups for the study are outlined in Table 8. Prior to be randomly placed in treatment groups, animals were weighed, and paw volume was measured. Once randomly assigned to treatment groups, on Day 0, animals in groups 2-9 were immunized by subcutaneous injection at the base of the tail. The immunization comprised 100 μL of the CFA+Collagen emulsion (prepared according to the method in Experimental Method A). Animals in group one received no immunization. Seven days after the first injection, animals in groups 2-9 received a booster immunization comprising 200 μL of the IFA+Collagen emulsion (prepared according to the method in Experimental Method A). Animals in group one did not receive a booster immunization.

TABLE 8
Experimental Groups.

| | | | | | | | | Paw volume, |
|---|---|---|---|---|---|---|---|---|
| | | Dose | | | | Dosing | | Clinical score, |
| | | Level | | | | volume | | and blood |
| Group | Treatment | (mg/kg) | CIA | N | Frequency | (ml/kg) | Route | collection |
| 1 | Vehicle | — | No | 8 | QD*21 | 10 | P.O. | Day 0, 6, 12, |
| 2 | Vehicle | — | Yes | 8 | QD*21 | 10 | P.O. | 13, 14, 15, 18, |
| 3 | Dexamethasone | 0.1 | Yes | 8 | QD*21 | 5 | P.O. | 19, and 21 (9 |
| 4 | GSK620 | 10 | Yes | 8 | QD*21 | 5 | P.O. | times); Plasma |
| 5 | Example 101 | 3 | Yes | 8 | BID*21 | 10 | P.O. | & serum on |
| 6 | Example 101 | 10 | Yes | 8 | BID*21 | 10 | P.O. | day 21 |
| 7 | Example 101 | 30 | Yes | 8 | BID*21 | 10 | P.O. | |
| 8 | Example 101 | 10 | Yes | 3 | BID*21 | 10 | P.O. | Plasma |
| | | | | | | | | collection on |
| | | | | | | | | day 0 |
| 9 | Example 101 | 10 | Yes | 3 | BID*21 | 10 | P.O. | Plasma |
| | | | | | | | | collection on |
| | | | | | | | | day 20 |

Starting on Day 0, animals in group 1 and 2 were administered daily doses of the vehicle per os (P.O.) for 21 days. Starting on Day 0, animals in groups 3 and 4 were administered daily doses of dexamethasone and GSK620, respectively, P.O. for 21 days.

Starting on Day 0, animals in groups 4-9 were administered twice daily doses of Example 101 according to the dosages outline in Table 8 for 21 days. The twice daily doses for animals in groups 4-9 were 8 hours apart (e.g., the first dose at 8:00 AM and the second dose at 4:00 PM).

Body weight of all animals was recorded daily for the 21-day period. Blind clinical scores on all four limbs were conducted for all animals in the study. Assessments were recording according to the clinical scoring system summarized in Table 9. Animals were assessed prior to receiving their first dose of the compound or control, and scores for each limb were combined to a total daily score for each animal. Clinical score assessments were performed on Day 0, 6, 12, 13, 14, 15, 18, 19, and 21 (for a total of 9 daily scores).

TABLE 9
Clinical scoring rubric

| Score | Condition |
|---|---|
| 0 | Normal |
| 1 | Mild, but definite redness and swelling of the ankle or wrist, |
| | or apparent redness and swelling limited to individual digits |
| | (regardless of the number of digits affected) |
| 2 | Moderate redness and swelling or ankle or wrist |
| 3 | Severe redness and swelling of the entire paw, including digits |
| 4 | Maximally inflamed limb with involvement of multiple joints |

Paw volume was recorded for each animal on Day 0, 6, 12, 13, 14, 15, 18, 19, and 21 (for a total of 9 measurements). To measure paw volume, animals were gently held with one hand while the hind paw was placed into a plethysmometer with the other hand. Lipid displacement by the paw was recorded and used to represent paw volume.

At the conclusion of the study (end of the 21-day period), animals were euthanized. Euthanasia occurred four hours after animals received their first dose on the morning of Day 21. Hind limbs were collected following euthanasia, with the left limb being used for H&E staining.

Blood samples were collected from animals in groups 1-7 following euthanasia. Blood samples were collected from animals in group 8 on Day 0, and blood samples were collected from group 9 on Day 20. Blood samples from group 8 and group 9 were collected at 0.25, 0.5, 1, 2, 4, 8, 10, 12, 13, and 24-hours following morning administration of their first dose of the compound. The second dose of the compound was administered after the 8-hour collection timepoint. All blood samples were collected at the eye socket and put into tubes containing EDTA-K2 as an anticoagulant and centrifuged at 4° C. at 4,000×g for 5 minutes. Plasma was transferred to a separate tube and stored at −75±15° C. for PK analysis. Pharmacokinetic analyses for Example 101 in Lewis rats were performed as described in Experimental Method B.

Blood was additionally collected from groups 1-7 via cardiac puncture following euthanasia on Day 21. Blood from cardiac puncture was placed in a 1.5 mL centrifuge tube without an anticoagulant and centrifuged at 8000 rcf for 15 minutes. Serum was divided into two separate tubes and stored at −75±15° C. One tube was used to analyze IgG1, and the other tube was reserved for additional analyses.

H&E-stained slides of designated tissues from animals were transferred to an independent pathologist for microscopic evaluation (SPFBio). Samples were assigned the histologic grades based on the severity of change from tissue where CIA was not induced. Histology grades assigned were as follows: grade 1 (minimal change), grade 2 (mild change), grade 3 (moderate change), grade 4 (marked change), and grade 5 (severe change).

Mild to moderate mixed cell inflammation was characterized by widespread accumulation of neutrophils and macrophages with lesser numbers of lymphocytes within one or more joint spaces, synovium, and periarticular soft tissues. Moderate inflammation was also associated with increased edema fluid, fibrin, and cellular debris. Marked mixed cell inflammation was characterized by dense, widespread accumulations of neutrophils and macrophages with lesser number of lymphocytes within one or more joint spaces, synovium and periarticular soft tissues. Marked inflammation was also associated with abundant edema fluid, fibrin, and cellular debris.

Mild to moderate granulation tissue (pannus) was associated with inflammation and characterized by widespread fibrovascular connective tissue that expanded the periarticular soft tissues of one or more joints, extended into joint spaces, was contiguous with areas of erosion/ulcer in articular cartilage and variably extended along the diaphysis. Marked granulation tissue (pannus) was associated with inflammation and was characterized by widespread fibrovascular connective tissue that expanded/replaced the periarticular soft tissues of one or more joints, extended into/expanded joint spaces, was contiguous with areas of erosion/ulcer in articular cartilage and/or increased eroded surface of bone, and variably extended along the diaphysis.

Minimal to moderate increased eroded surface of bone (bone resorption) affected both trabecular bone, and cortical bone was characterized by irregular, scalloped bone surfaces, increased numbers of osteoclasts, and/or disruption of lamellae involving one bone. Instances of minimal increased eroded surface were not associated with fragments of bone contiguous and/or surrounded by granulation tissue. Moderate to marked increased eroded surface of bone (bone resorption) affected both trabecular bone and cortical bone and was characterized by irregular, scalloped bone surfaces, increased numbers of osteoclasts, and/or disruption of lamellae involving one or more bones. In some areas, increased eroded surfaces were contiguous with areas of erosion/ulcer in the articular cartilages. Instance of marked increased eroded surface were often associated with fragments of bone contiguous with and/or surrounded by granulation tissue.

Mild to moderate erosion/ulcer in the articular cartilages was characterized by local area of incomplete (erosion) loss of the articular cartilage at one or more bones. Moderate to marked erosion/ulcer in articular cartilages was characterized by multifocal areas of incomplete (erosion) or complete (ulcer) loss of articular cartilage at one or more bones. This observation was typically most pronounced at joint margins (transition zone). The ulceration was often associated with inflammation and granulation tissue extending through the defect to the subchondral bone.

Mild to moderate increased periosteal bone (periosteal bone formation) was characterized by local areas of increased bone matrix and numerous prominent osteoblasts lining trabecular surfaces along the metaphysis of one or more bones. In some areas, increased periosteal bone was contiguous with increased eroded surface and at articular cartilage. Marked increased periosteal bone (periosteal bone formation) was characterized by extensive areas of increased bone matrix and numerous prominent osteoblasts lining trabecular surfaces along the metaphysis and/or diaphysis of one or more bones. In some areas, increased periosteal bone was contiguous with increased eroded surface and/or erosion/ulcer at articular cartilage.

All data measured and recorded was analyzed using SPSS 16.0 software. Groups were compared using one-way ANOVA and significance was set at p<0.05.

### Example 137: Experimental Method D—Cellular Activity in Human Peripheral Blood Mononuclear Cells

The impact of Examples 23, 80, and 101 on IL-17A, IL-22, and CXCL10 levels was analyzed in cryopreserved human peripheral blood mononuclear cells (PBMC) by ELISA.

PBMCs were thawed and cultured in RPMI-1640 medium supplemented with 10% fetal bovine serum. For IL-17A and IL-22 assays, Examples 23, 80, and 101 were serially diluted from 10 mM in DMSO and further diluted in assay media to 100× the required final concentration, of which, 2 μL was added per well to a round-bottom 96-well tissue culture plate where the final DMSO concentration per well was 0.1%. CD2, CD3, and CD28 antibody-coated beads from the T-cell activation/expansion kit (Miltenyi Biotec) were added to the PBMC at a bead-to-cell ratio of 1:2. The mixture of PBMCs and beads were seeded into the wells of the round-bottom 96-well plate at a concentration of 2×10⁵ cells/well in a total volume of 200 μL. The cells were cultured for 72 hours at 37° C. and 5% CO₂ and then supernatants were collected and IL-17A and IL-22 levels were measured by ELISA. For the CXCL10 assay, Examples 23, 80, and 101 were serially diluted from 10 mM in DMSO and further diluted in assay media to 100× the required final concentration, of which 2 μL was added per well to a 96-well tissue culture plate. The final compound concentration ranged from 30,000 nM to 0.005 nM and the final DMSO concentration per well was 0.3%. PBMCs were seeded at approximately 2×10¹ cells/well in the wells of the 96-well tissue culture plate in a total volume of 190 μL. The plate was incubated for 1 hour at 37° C. and 5% CO₂. The cells were then stimulated with IFN-γ (15 ng/mL final concentration) in a volume of 10 μL. The cells were then cultured for 24 hours at 37° C. and 5% CO₂. The supernatant was collected and CXCL10 levels were measured by ELISA.

### Example 138: Experimental Method E—Unilateral Urethral Obstruction in Animals Treated with Example 101 and Vehicle

Unilateral Urethral Obstruction (UUO) is a complex model of renal fibrosis (RF) disease. RF is a common outcome in progressive chronic kidney diseases, characterized by excessive formation of internal scar tissue. Surgical obstruction of urine flow can be used to trigger this disease in rodents. The UUO model allows for evaluation of potential drugs as treatment for RF.

The UUO model in rats was used to assess the efficacy of oral delivery of Example 101 at a 3.0 mg/mL concentration (30 mg/kg dose). Experimental groups for the study are outlined in Table 10. Prior to be randomly placed in treatment groups, animals were weighed. Once randomly assigned to treatment groups, on Day 0, animals in groups 2-3 were subjected to UUO surgery. Briefly, animals were anesthetized using a Zoletil/Xylazine mixture (20:1, v:v) for a final dose of 25 mg/kg. The operation area was disinfected with 75% alcohol before shaving. Rats in UUO group were exposed left ureter through a small incision on the left side of the abdomen and ligated with 3-0 silk at two locations about 0.5 cm below the renal hilum. The ureters of sham rats in Group 1 were manipulated but not ligated.

TABLE 10
Experimental Groups.

| | | | | | | Clinical observation, body |
|---|---|---|---|---|---|---|
| | | | | | Dose | weight measurement, |
| | | Dose Level | | | volume | clinical nephropathy score, |
| Group | Treatment | (mg/kg) | UUO | N | (ml/kg) | blood and tissue collection |
| 1 | Sham | — | No | 8 | 10 | Body Weight measured Days −1- |
| 2 | Vehicle, QD | — | Yes | 8 | 10 | 14; Clinical observation |
| 3 | Example | 30 | Yes | 8 | 10 | assessed Days 0-14; Clinical |
| | 101, BID | | | | | Scores determined, and |
| | | | | | | Blood and Tissue collected |
| | | | | | | on Day 14 |

Starting on Day 0, animals in group 2 were administered daily doses of the vehicle for 14 days. Starting on Day 0, animals in group 3 were administered daily doses of Example 101 (30 mg/kg, BID at 0 and 8 hours) for 14 days.

Body weight of all animals was recorded daily for the 14-day period. Clinical signs for all animals were monitored and recorded daily. Blind clinical nephropathy scores were conducted for all animals in the study. Assessments were recording according to the clinical scoring system summarized in Table 11. Glomerulosclerosis, interstitial nephritis, collagen fiber deposition, nephropathy were each assessed for all experimental rats, and a total clinical score was determined for each rat. The scoring system represented the degree or percentage of lesions in the whole kidney. Clinical score assessments were performed on Day 14.

TABLE 11
Clinical scoring rubric

| Score | Prevalence of clinical symptom in whole kidney |
|---|---|
| 0 | Normal, <15% |
| 1 | Mild, 15%-≤30% |
| 2 | Moderate, 30%-≤50% |
| 3 | Severe, 50%-≤80% |
| 4 | Very severe, ≥80% |

Serum for blood biochemistry analysis was collected into centrifuge tubes without coagulant four hours after final dosing. Blood plasma samples from group 3 was collected into tubes with K₂EDTA four hours after final dosing. At the conclusion of the study (end of the 14-day period), animals were euthanized. Kidney tissue was collected four hours after final dosing. Ligated side kidney was divided into three copies 1/3 fix into 10% formalin for H&E analysis, 1/3 into RNA for PCR analysis of tissue biomarkers Col1a1, TGF-b1, MCP-1, IL-1b, IL-6, IL-17, TNF-a, and Timp1, and 1/3 for hydroxyproline assay analyzed by ELISA. RNA was extracted from tissue using the RNeasy mini kit (Qiagen). RNA concentration was measured using a NanoDrop One Spectrophotometer (ThermoFisher) and converted to cDNA using a High-Capacity RNA-to-cDNA kit (Invitrogen). RT-PCR was performed to measure levels of mRNA of different targets using TaqMan® Gene Expression Master Mix and TaqMan® probe/primer sets on a QuantStudio 6 Real-Time PCR System (ThermoFisher). Fold change in target gene expression was determined using the ΔΔCt method.

Concentration of Example 101 in plasma samples was determined by liquid chromatography-mass spectrometry (LC-MS/MS) using a Shimadzu HPLC (DGU-20A5R; LC-30AD; SIL-30AC; and Rack changer II) and an AB API40000 LC/MS/MS instrument. Samples were analyzed on a Raptor Biphenyl 2.7 μm 50×2.1 mm column. Solution A in the mobile phase was 95% water (0.1% formic acid) and solution B was 95% acetonitrile in water (0.1% formic acid), injection volume was 20 μL, and flow rate was 0.5 mL/min.

To prepare samples for analysis, serial concentrations were prepared by diluting the stock solution of analyte with a 50% acetonitrile in water solution. 5 μL working solutions (5, 10, 20, 50, 100, 500, 1000, 5000, and 10000 ng/mL) were added to 50 μL of the blank SD rat plasma to achieve calibration standards of ˜0.5 to 1000 ng/mL (0.5, 1, 2, 5, 10, 50, 100, 500, and 1000 ng/mL) in a total volume of 55 μL. Four quality control samples at 1 ng/mL, 2 ng/mL, 50 ng/mL, and 800 ng/mL for plasma were prepared independently of those used for the calibration curves. These QC samples were prepared on the day of analysis in the same way as calibration standards.

55 μL of standards, 55 μL of QC samples and 55 μL of unknown samples (50 μL of unknown rat plasma with 5 μL of blank solution) were added to 200 μL of acetonitrile containing IS mixture for precipitating protein respectively. The samples were then vortexed for 30 s. After centrifugation at 4° C., 4000 rpm for 15 min, the supernatant was diluted 3 times with water. 20 μL of diluted supernatant was injected into the LC/MS/MS system for quantitative analysis.

All data measured and recorded was analyzed using SPSS 16.0 software. Groups were compared using one-way ANOVA and significance was set at p<0.05.

### Example 139: Pharmacokinetics of Example 101 in Beagles

Animals were administered solutions with different concentrations of Example 101 either intravenously (group 1) or orally (group 2) as outlined in Experimental Method B.

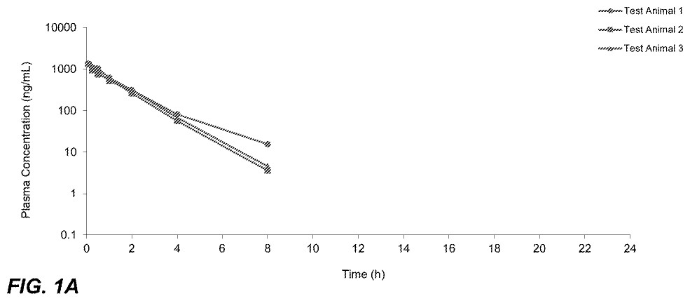

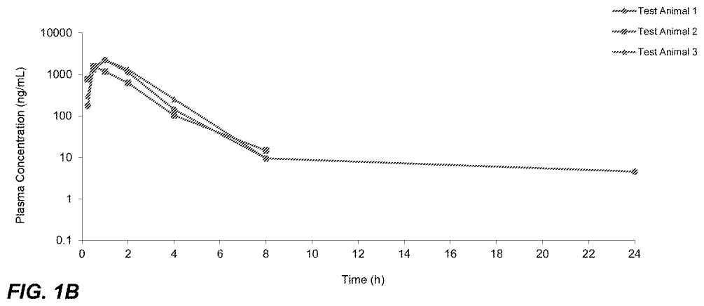

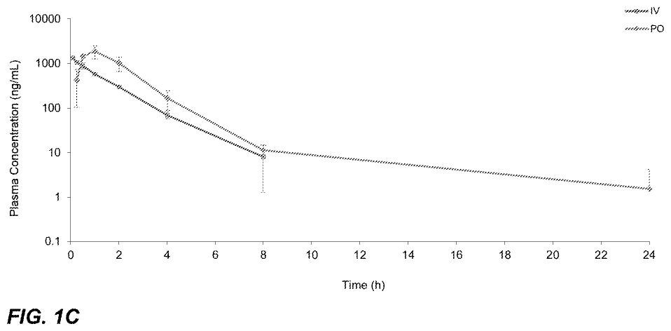

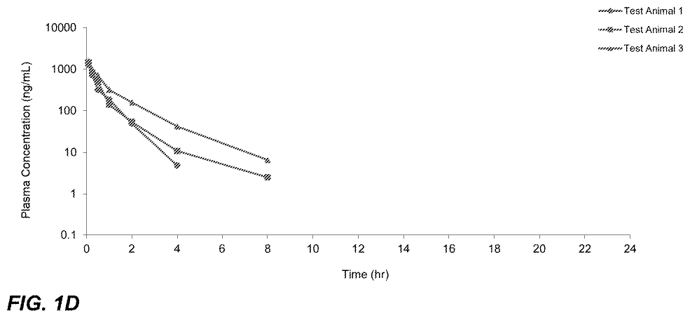

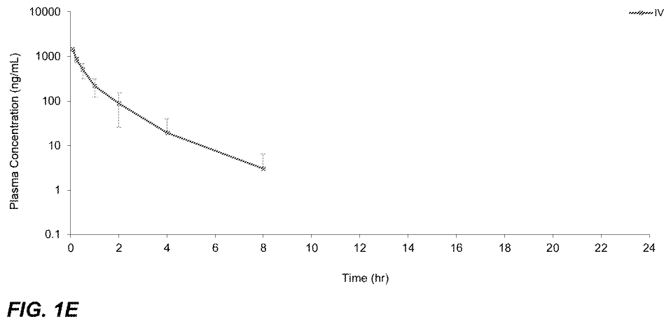

FIG. 1A shows a graph plotting the plasma concentration of Example 101 in group 1 as a function of time. Decreases in plasma concentration were consistent across all animals and timepoints following intravenous administration, with only animal 1 exhibiting slightly increased levels of Example 101 at 8 h post-administration.

FIG. 1B shows a graph plotting the plasma concentration of Example 101 in group 2 as a function of time. All animals showed a similar increase in Example 101 plasma concentration following oral administration. At approximately 1 h post-administration, plasma concentrations began to steadily decrease for the next 7 h in all animals.

FIG. 1C shows a graph plotting the mean plasma concentration of Example 101 in groups 1 and 2, respectively, as a function of time.

Tables 12A-12B depict plasma concentration of Example 101 in animals from PK study 1 receiving Example 101 via intravenous administration. Tables 11C-11 D depict plasma concentration of Example 101 in animals from PK study 1 receiving Example 101 via oral administration.

TABLE 12A
Plasma concentration of Example 101
in PK study 1 after IV administration

| | Concentration (ng/mL) | | |
|---|---|---|---|
| | Test | Test | Test |
| Time (h) | Animal 1 | Animal 2 | Animal 3 |
| 0.083 | 1340 | 1320 | 1370 |
| 0.25 | 1120 | 954 | 1110 |
| 0.5 | 1040 | 747 | 867 |
| 1 | 623 | 580 | 520 |
| 2 | 314 | 265 | 313 |
| 4 | 81.6 | 56.5 | 68.0 |
| 8 | 15.7 | 3.65 | 4.61 |
| 24 | 0.00 | 0.00 | 0.00 |

TABLE 12B
Average plasma concentration of Example
101 in PK study 1 after IV administration

| Time (h) | Mean (ng/mL) | SD (ng/mL) | CV (%) |
|---|---|---|---|
| 0.083 | 1343 | 25 | 1.87 |
| 0.25 | 1061 | 93 | 8.77 |
| 0.5 | 885 | 147 | 16.6 |
| 1 | 574 | 52 | 9.01 |
| 2 | 297 | 28 | 9.42 |
| 4 | 68.7 | 12.6 | 18.3 |
| 8 | 8.0 | 6.7 | 83.9 |
| 24 | 0.00 | NA | NA |

TABLE 12C
Plasma concentration of Example 101 in
PK study 1 after oral administration

| | Concentration (ng/mL) | | |
|---|---|---|---|
| | Test | Test | Test |
| Time (h) | Animal 1 | Animal 2 | Animal 3 |
| 0.25 | 173 | 775 | 309 |
| 0.5 | 1480 | 1550 | 1320 |
| 1 | 2220 | 1180 | 2240 |
| 2 | 1130 | 621 | 1310 |
| 4 | 142 | 103 | 254 |
| 8 | 9.51 | 14.8 | 9.52 |
| 24 | 4.58 | 0.00 | 0.00 |

TABLE 12D
Average plasma concentration of Example 101
in PK study 1 after oral administration

| Time (h) | Mean (ng/mL) | SD (ng/mL) | CV (%) |
|---|---|---|---|
| 0.25 | 419 | 316 | 75.3 |
| 0.5 | 1450 | 118 | 8.13 |
| 1 | 1880 | 606 | 32.3 |
| 2 | 1020 | 357 | 35.0 |
| 4 | 166 | 78 | 47.1 |
| 8 | 11.3 | 3.1 | 27.1 |
| 24 | 1.53 | 2.64 | 173 |

The clinical observations for PK study 1 were as follows.

The clinical observations for test animal 1 in group 1 were as follows: The animal was inactive and lying in the cage 5 minutes following administration of the Example 101 solution. Vomitus with solid food and soft feces was observed at 0.25 h timepoint. Twitching and rapid breathing was also observed at 0.25 h timepoint. The animal was able to stand up, but the movement was unstable at the 0.5 h timepoint. The animal remained inactive during the 2-24 h timepoints following administration of the Example 101 solution. Watery feces with blood were observed at 24 h timepoint. Animal was inactive at 17:00, 11/15/2022. Animal was recovered at 10:00, 11/16/2022.

The clinical observations for test animal 2 in group 1 were as follows: The twitching of hindlimbs was observed approximately 1 min after administration of the Example 101 solution. Soft feces and rapid breathing were observed at the 0.25 h timepoint. The animal was able to stand up, but movement was unstable at the 0.5 h timepoint. Vomitus with solid food was observed at 1 h timepoint and the animal had not recovered by the 4 h timepoint. Torpidity and watery feces with blood were observed at 24 h timepoint. Animal was recovered at 17:00, 11/15/2022.

The clinical observations for test animal 3 in group 1 were as follows: Slight twitching and lacrimation was observed despite the animal being immobile and lying in the cage approximately 5 minutes after administration of the Example 101 solution. Urinary incontinence and rapid breathing were observed at 0.25 h timepoint. The animal was not recovered at the 0.5 h timepoint. Slight twitching disappeared at the 1 h timepoint. The animal was inactive and lying in the cage at the 2 h timepoint. Animal had not recovered by 4 h timepoint, and tremors were observed. At the 4 h timepoint, the animal's temperature was 36.4° C. The animal was able to move normally at the 6 h timepoint, but it remained inactive. At the 6 h timepoint, the animal's temperature was 38.1° C. The animal remained inactive and lying in the cage at the 8 h timepoint. The animal was recovered by the 24 h timepoint.

The clinical observations for test animal 1 of group 2 were as follows: Loose feces were observed at the 4 h timepoint.

No clinical observations were made for test animals 2 or 3 of group 2.

Tables 12E and 12F show the pharmacokinetic profiles of Example 101 after intravenous administration and Tables 12G and 12H show the pharmacokinetic profiles of Example 101 after oral administration (Table 12B) in beagle dogs as outlined in Table 7A in Experimental Method B.

TABLE 12E
PK profile of Example 101 following intravenous administration

| Pharmacokinetic | | Test | Test | Test |
|---|---|---|---|---|
| Parameter | Unit | Animal 1 | Animal 2 | Animal 3 |
| Cl_obs | mL/min/kg | 7.94 | 9.67 | 8.91 |
| T_{1/2} | h | 1.42 | 0.976 | 0.992 |
| C₀ | ng/mL | 1465 | 1551 | 1521 |
| AUCₗₐₛₜ | h*ng/mL | 2066 | 1718 | 1864 |
| AUC_{Inf} | h*ng/mL | 2099 | 1723 | 1870 |
| AUC_{_{—}}_{% Extrap}_obs | % | 1.54 | 0.298 | 0.353 |
| MRT_{Inf}_obs | h | 1.54 | 1.29 | 1.35 |
| AUCₗₐₛₜ/D | (ng · h/mL)/ | 2066 | 1718 | 1864 |
| | (mg/kg) | | | |
| Vₛₛ_obs | L/kg | 0.734 | 0.747 | 0.719 |

TABLE 12F
Average PK profile of Example 101 following
intravenous administration

| Pharmacokinetic | | | | |
|---|---|---|---|---|
| Parameter | Unit | Mean | SD | CV (%) |
| Cl_obs | mL/min/kg | 8.84 | 0.87 | 9.82 |
| T_{1/2} | h | 1.13 | 0.25 | 22.5 |
| C₀ | ng/mL | 1512 | 44 | 2.89 |
| AUCₗₐₛₜ | h*ng/mL | 1883 | 175 | 9.30 |
| AUC_{Inf} | h*ng/mL | 1897 | 189 | 9.98 |
| AUC_{_{—}}_{% Extrap}_obs | % | 0.73 | 0.70 | 96.0 |
| MRT_{Inf}_obs | h | 1.39 | 0.13 | 9.50 |
| AUCₗₐₛₜ/D | (ng · h/mL)/(mg/kg) | 1883 | 175 | 9.30 |
| Vₛₛ_obs | L/kg | 0.733 | 0.014 | 1.92 |

TABLE 12G
PK profile of Example 101 following oral administration

| Pharmacokinetic | | Test | Test | Test |
|---|---|---|---|---|
| Parameter | Unit | Animal 1 | Animal 2 | Animal 3 |
| T_{1/2} | h | NA | 1.15 | 0.845 |
| Tₘₐₓ | h | 1.00 | 0.500 | 1.00 |
| Cₘₐₓ | ng/mL | 2220 | 1550 | 2240 |
| AUCₗₐₛₜ | h*ng/mL | 4516 | 2930 | 4998 |
| AUC_{Inf} | h*ng/mL | NA | 2955 | 5010 |
| AUC_{_{—}}_{% Extrap}_obs | % | NA | 0.831 | 0.232 |
| MRT_{Inf}_obs | h | NA | 1.62 | 1.83 |
| AUCₗₐₛₜ/D | (ng · h/mL)/ | 903 | 586 | 1000 |
| | (mg/kg) | | | |
| F | % | 48.0 | 31.1 | 52.8 |

TABLE 12H
Average PK profile of Example 101 following oral administration

| Pharmacokinetic | | | | CV |
|---|---|---|---|---|
| Parameter | Unit | Mean | SD | (%) |
| T_{1/2} | h | 1.00 | NA | NA |
| Tₘₐₓ | h | 0.83 | 0.29 | 34.6 |
| Cₘₐₓ | ng/mL | 2003 | 393 | 19.6 |
| AUCₗₐₛₜ | h*ng/mL | 4148 | 1082 | 26.1 |
| AUC_{Inf} | h*ng/mL | 3982 | NA | NA |
| AUC_{_{—}}_{% Extrap}_obs | % | 0.531 | NA | NA |
| MRT_{Inf}_obs | h | 1.73 | NA | NA |
| AUCₗₐₛₜ/D | (ng · h/mL)/(mg/kg) | 830 | 216 | 26.1 |
| F | % | 44.0 | 11.4 | 25.9 |

Tables 12I and 12J provide the plasma concentration of Example 101 over time in animals in PK study 2 with the HPP3CD based formulation.

TABLE 12I
Plasma concentration of Example 101 in PK study 2

| | Concentration (ng/mL) | | |
|---|---|---|---|
| | Test | Test | Test |
| Time (h) | Animal 1 | Animal 2 | Animal 3 |
| 0.083 | 1270 | 1460 | 1580 |
| 0.25 | 721 | 820 | 985 |
| 0.5 | 322 | 498 | 702 |
| 1 | 187 | 140 | 320 |
| 2 | 49.3 | 54.1 | 159 |
| 4 | 4.83 | 10.8 | 42.1 |
| 8 | 0.00 | 2.50 | 6.58 |
| 24 | 0.00 | 0.00 | 0.00 |

TABLE 12J
Average plasma concentration of Example 101 in PK study 2

| Time (h) | Mean (ng/mL) | SD (ng/mL) | CV (%) |
|---|---|---|---|
| 0.083 | 1437 | 156 | 10.9 |
| 0.25 | 842 | 133 | 15.8 |
| 0.5 | 507 | 190 | 37.5 |
| 1 | 216 | 93 | 43.3 |
| 2 | 87 | 62 | 70.9 |
| 4 | 19.2 | 20.0 | 104 |
| 8 | 3.03 | 3.32 | 110 |
| 24 | BLOQ | NA | NA |

FIGS. 1D-1E show graphs plotting the plasma concentration of Example 101 in animals as a function of time in PK study 2. Plasma concentrations decreased similarly over the first 2 h post-administration in all animals receiving Example 101. The clinical observations for PK study 2 were as follows.

One animal receiving 0.5 mg/mL of Example 101 via intravenous administration had soft feces 8 hours after administration. No other clinical observations were made over the course of PK study 2.

Tables 12K and 12L shows the pharmacokinetic profile from PK study 2 of Example 101 after intravenous administration in beagle dogs as outlined in Table 7B in Experimental Method B.

TABLE 12K
PK profile of Example 101 following intravenous administration

| Pharmacokinetic | | Test | Test | Test |
|---|---|---|---|---|
| Parameter | Unit | Animal 1 | Animal 2 | Animal 3 |
| Cl_obs | mL/min/kg | 23.1 | 19.6 | 12.1 |
| T_{1/2} | h | 0.573 | 1.41 | 1.33 |
| C₀ | ng/mL | 1683 | 1945 | 1998 |
| AUCₗₐₛₜ | h*ng/mL | 719 | 844 | 1367 |
| AUC_{Inf} | h*ng/mL | 723 | 850 | 1380 |
| AUC_{_{—}}_{% Extrap}_obs | % | 0.552 | 0.599 | 0.915 |
| MRT_{Inf}_obs | h | 0.606 | 0.752 | 1.20 |
| AUCₗₐₛₜ/D | (ng · h/mL)/ | 719 | 844 | 1367 |
| | (mg/kg) | | | |
| Vₛₛz_obs | L/kg | 0.838 | 0.886 | 0.868 |

TABLE 12L
PK profile of Example 101 following intravenous administration

| Pharmacokinetic | | | | |
|---|---|---|---|---|
| Parameter | Unit | Mean | SD | CV (%) |
| Cl_obs | mL/min/kg | 18.3 | 5.6 | 30.8 |
| T_{1/2} | h | 1.10 | 0.46 | 41.9 |
| C₀ | ng/mL | 1875 | 169 | 9.01 |
| AUCₗₐₛₜ | h*ng/mL | 977 | 344 | 35.2 |
| AUC_{Inf} | h*ng/mL | 984 | 348 | 35.4 |
| AUC_{_{—}}_{% Extrap}_obs | % | 0.689 | 0.197 | 28.7 |
| MRT_{Inf}_obs | h | 0.85 | 0.31 | 36.2 |
| AUCₗₐₛₜ/D | (ng · h/mL)/(mg/kg) | 977 | 344 | 35.2 |
| Vₛₛz_obs | L/kg | 0.864 | 0.024 | 2.76 |

Without being bound by any particular theory, animals exhibited fewer clinical observations in response to Example 101 when prepared in HPβCD relative to Example 101 prepared in 20% propylene glycol/20% Vitamin E TPGS/60% water. Without being bound by any particular theory, these studies show that Example 101 is capable of being administered orally and intravenously and well tolerated. Additionally, when Example 101 was prepared in HPβCD, some animals exhibited fewer reactions shortly after administration of Example 101. Without being bound by any particular theory, this also suggests that the clinical observations noted in PK study 1 may be a result of, either in part or in its entirety, the formulation Example 101 was prepared in (i.e., 20% propylene glycol/20% Vitamin E TPGS/60% water).

### Example 140: Collagen-Induced Arthritis in Animals Treated with Example 101 and Vehicle

The collagen-induced arthritis (CIA) model in rats was used to assess the efficacy of oral delivery of Example 101 at three dosages (3 mg/kg, 10 mg/kg, and 30 mg/kg). Example 101 suspensions were prepared according to the procedures set out in Experimental Method A. Evaluation of the effect of different dosages of Example 101 compared to vehicle and controls in collagen-induced arthritis in rats was undertaken in accordance with the protocol set out in Experimental Method C.

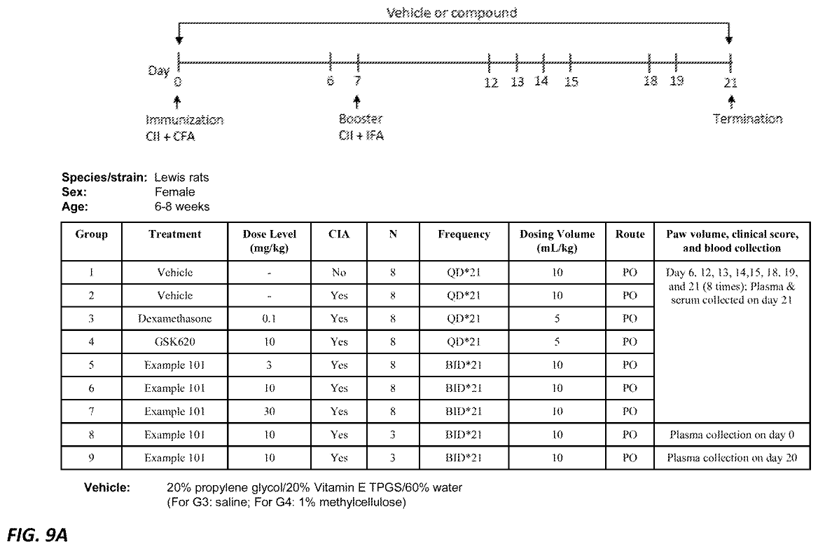

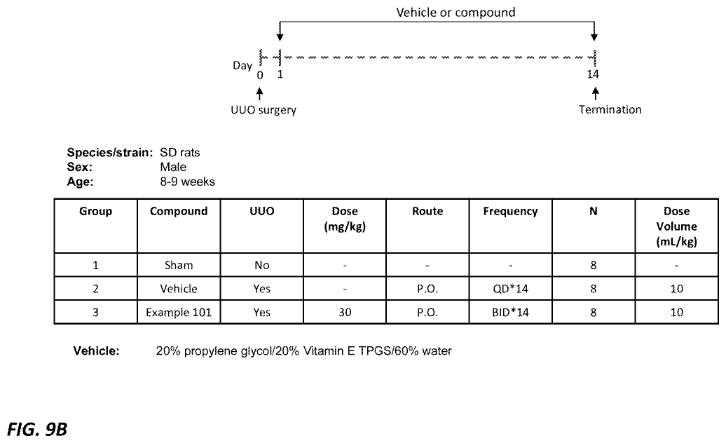

Arthritis was induced in Lewis rats by immunizing animals with the CFA+Collagen emulsion via subcutaneous injection at the base of the tail on day 0. Seven days after the first immunization, animals received a booster immunization with the IFA+Collagen emulsion via subcutaneous injection at the base of the tail. Animals not subjected to CIA did not receive the immunizations. A design of the CIA study is depicted in FIG. 9A.

Starting on day 0, animals in group 1 and 2 were administered daily doses of the vehicle per os (P.O.) for 21 days. On day 0, animals in groups 3 and 4 were administered daily doses of dexamethasone and GSK620, respectively, P.O. for 21 days. Starting on day 0, animals in groups 4-9 were administered twice daily doses of Example 101 (3 mg/kg, 10 mg/kg, or 30 mg/kg) for 21 days. The twice daily doses for animals in groups 4-9 were 8 hours apart (e.g., the first dose at 8:00 AM and the second dose at 4:00 PM).

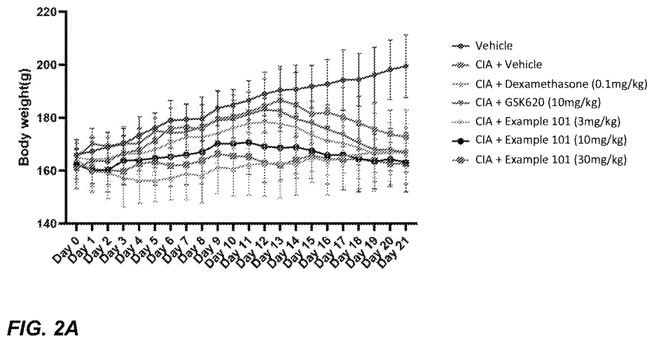

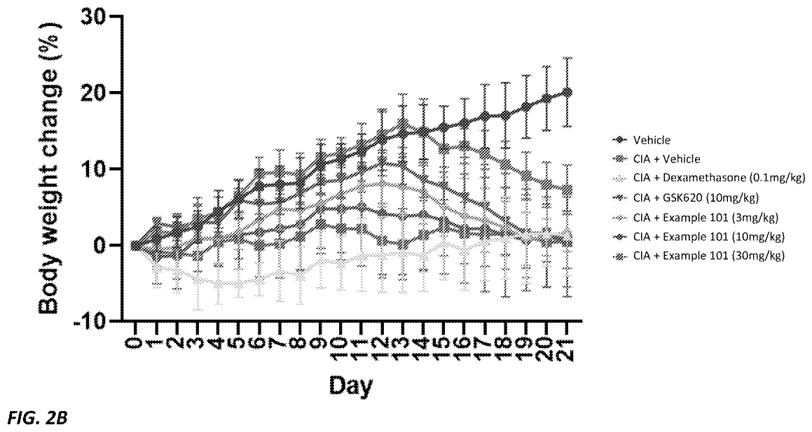

Animal body weight was measured daily over the course of the study (see Tables 13A-13D, FIGS. 2A-2B). CIA-induced animals receiving no treatment (group 2) exhibited a modest weight gain over the first 13 days followed by weight loss over the remaining study days. Despite the weight loss over the last seven days of the study, all animals in group 2 had gained weight when day 21 weights were compared to starting weights on day 0. In contrast, animals where CIA was not induced, and no treatment was administered (group 1) steadily gained weight over the course of the study and animal weight on day 21 was significantly increased relative to group 2 (p<0.01). Animals receiving dexamethasone did not exhibit weight gains over the first 13 days of the study, exhibiting significantly reduced weight (p<0.01) over days 4-7 and 9-17 relative to animals in group 2. Ultimately, only 4/8 animals receiving dexamethasone after induction of CIA exhibited a weight gain by the end of the study, whereas 2/8 animals lost weight, and 2/8 animals weighed the same at the end of the study as their starting weight. Animals receiving GSK620 (group 4) exhibited modest weight gains over the first 13 days of the study followed by weight loss, similar to animals in group 2. At day 21, 4/8 animals had gained weight after receiving daily administration of GSK620 following induction of CIA.

Animals receiving the low dose of Example 101 (3 mg/kg, group 5) exhibited a weight gain over the first 13 days of the study, similar to animals in groups 2 and 4, followed by weight loss over the remaining days. On day 16 and 17, animal weight was significantly decreased (p<0.05) relative to animal weight in group 2, but by day 21 there was no significant difference in animal weight. In group 5, 7/8 animals exhibited increased weight at the end of the study. Animals in groups 6 and 7 (Example 101 10 mg/kg and 30 mg/kg, respectively) exhibited modest weight gains over the first 10-11 days of the study, followed by weight loss. Animals in group 6 exhibited weights that were significantly decreased relative to group 2 on days 6-7 and 10-18 (p<0.01), and on days 9 and 19-21 (p<0.05). Only 3/8 animals in group 6 gained weight over the course of the study, whereas 5/8 either gained no weight or lost weight. Similarly, animals in group 7 exhibited weights that were significantly decreased relative to group 2 on days 6-7 and 9-18 (p<0.01), and on days 5 and 19-21 (p<0.05). Similar to group 6, only 3/8 animals gained weight over the course of the study, and 5/8 either lost weight or gained no weight.

Comparing animal weights on day 21 to day 0, animals where CIA was not induced experienced a 20.1% increase in body weight (Table 12D). In contrast, when CIA was induced by no treatment was provided, animals experienced a 7.4% increase in body weight. Animals in the dexamethasone and Example 101 (3 mg/kg) groups exhibited a 1.3% and 1.7% increase in body weight, respectively. Animals in the GSK620 treatment group exhibited a 0.8% increase in body weight, and animals in the Example 101 10 mg/kg and 30 mg/kg groups experienced weight changes of approximately 0.5% at the end of the study.

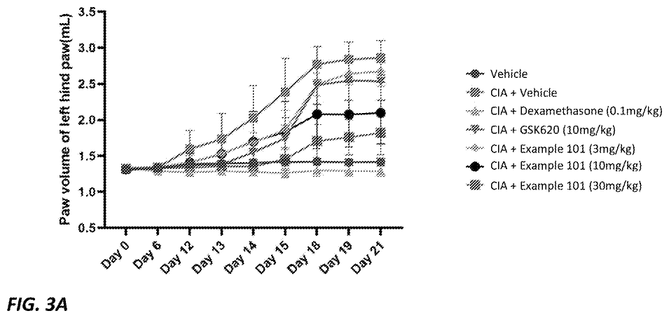

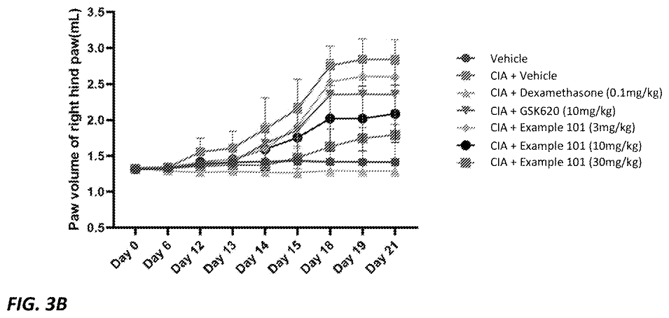

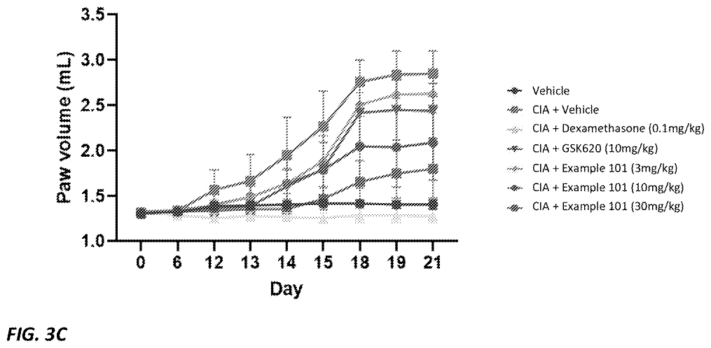

Animals where CIA was induced exhibited a consistent increase in paw volume in both the right and left hind paw starting on day 6 and continuing throughout the study (Tables 14A-14D, FIGS. 3A-3C). There was generally no significant difference in paw volumes for the left hind paw relative to the right hind paw. Tables 14A and 14C show that paw volume in animals with CIA was significantly increased relative to animals where CIA was not induced on day 15 (both paws, p<0.05) and on days 18, 19, and 21 (both paws, p<0.01). Dexamethasone was effective at controlling paw volume, and animals receiving dexamethasone had paw volumes similar to uninduced animals, and these paw volumes were significantly decreased relative to animals in group 2 on days 14 (left paw, p<0.05), 15 (left paw, p<0.01; right paw, p<0.05), and 18, 19, and 21 (both paws, p<0.01). Both GSK620 (group 4) and Example 101 3 mg/kg (group 5) were less effective at preventing an increase in paw volume over the course of the study, with paw volumes similar to CIA induced animals receiving vehicle. Animals in group 6 (Example 101 10 mg/kg) exhibited an increase in paw volume over the study, but this increase was significantly decreased relative to group 2 on days 19 and 21 (both paws, p<0.05). The high dose of Example 101 (30 mg/kg, group 7) proved the most effective dose at preventing an increase in paw volume, and paw volume was significantly reduced relative to group 2 at day 15 (left paw, p<0.05) and days 18, 19, 21 (both paws, p<0.01). Paw volume increases were delayed in groups 6 and 7, with appreciable increases not occurring until day 18 for animals in both groups. Without being bound by any theory, while vehicle-treated, GSK620-treated, and animals treated with the low dose of Example 101 developed active arthritis as evidenced by increased paw volume at day 21, administration of Example 101 ameliorated joint inflammation in a dose-dependent manner (i.e., the higher doses 10 mg/kg and 30 mg/kg demonstrated the lowest paw volumes).

Tables 14B and 14D show percent change in rat hind paw volume over the course of the study. As noted, there was no appreciable differences in paw volumes for the left hind paw relative to the right hind paw. Paw volumes remained constant for all groups on day 6, prior to the booster immunization. Animals in group 2 exhibited an approximately 20% increase in paw volume in both paws on day 12, and paw volume steadily increased until days 18-19, where paw volume plateaued at an increase of approximately 115% relative to day 0. Dexamethasone was effective at preventing any change in paw volume throughout the entire 21-day period. Animals receiving GSK620 exhibited modest increases in paw volume until day 14, when both paws had paw volumes that were approximately 20% greater than day 0, and paw volume continued to increase until it plateaued at day 18, with a paw volume that was between 75-90% increased relative to day 0. The low dose Example 101 treatment (3 mg/kg, group 5) performed similarly to GSK620, with paw volume noticeably increasing at around day 14, and continuing to increase until it plateaued at days 18-19, with a paw volume that was increased approximately 90% relative to day 0. The higher doses of Example 101 were more effective at controlling paw volume. While animals receiving the 10 mg/kg Example 101 dose also exhibited an increase in paw volume around day 14, paw volume at day 18 plateaued at an increased volume of approximately 50% relative to day 0. The higher dose of Example 101 (30 mg/kg, group 7) proved most effective, with paw volume noticeably increasing around day 19, and plateauing at an increased volume of approximately 35% relative to day 0.

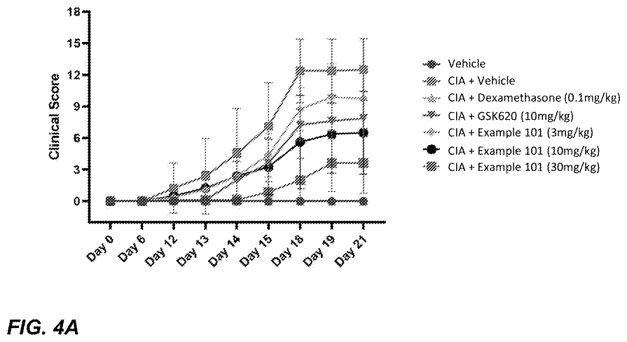

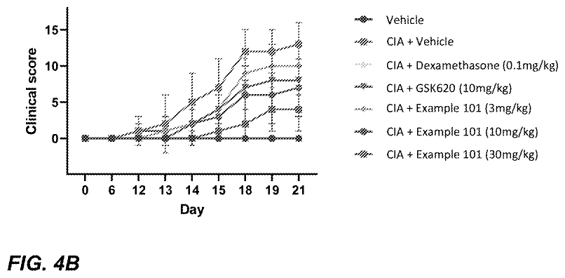

Tables 15A-15E and FIGS. 4A-4B show clinical scores and observations for animals in the different treatment groups. In FIGS. 4A-4B, Group 3 scored the same as Group 1, and data points for Group 3 may be hidden behind data points for Group 1. Clinical scores were recording according to Table 9, outlined in Experimental Method C. No animals exhibited any signs of disease prior to day 12. On day 12, 2/8 in group 2 exhibited clinical symptoms, with one animal showing disease in both hind limbs, and the other showing disease in the left forelimb and both hind limbs. No animals in group 3 (dexamethasone) or group 4 (GSK620) showed any signs of disease on day 12. Three animals in group 5 (Example 101, 3 mg/kg) exhibited signs of disease in either the right forelimb or the left forelimb on day 12. Likewise, 3/8 animals in group 6 (Example 101, 10 mg/kg) exhibited signs of disease in either the left hind limb or the right hind limb on day 12. One animal in group 7 (Example 101, 30 mg/kg) exhibited signs of disease in the right hind limb on day 12. Nevertheless, Example 101 at all three concentrations had lower clinical scores and, as evidenced by clinical scores at day 21, administration of Example 101 ameliorated joint inflammation in a dose-dependent manner (i.e., with the higher doses of 10 mg/kg and 30 mg/kg demonstrating the lowest clinical scores).

Animals in group 2 continued to exhibit symptoms, with 4/8 exhibiting symptoms on day 13, 6/8 exhibiting symptoms by day 14, and 8/8 animals exhibiting symptoms of disease by day 15. No animals in group 3 (dexamethasone) ever received a score indicating that they exhibited symptoms of disease. In group 4 (GSK620), on day 13 1/8 animals exhibited disease, and on day 14, 5/8 animals exhibited disease. By day 15, 7/8 animals exhibited disease, and on day 18 all animals were showing signs of disease. In group 5 (Example 101, 3 mg/kg) 5/8 animals were showing disease by day 13, and all animals were exhibiting disease on day 14. In group 6 (Example 101, 10 mg/kg) 4/8 animals were showing disease by day 13, 7/8 animals were showing disease on day 15, and all animals were exhibiting disease on day 19. In group 7 (Example 101, 30 mg/kg) 4/8 animals were showing disease by day 15, 5/8 animals were showing disease on day 16, and 7/8 animals were exhibiting disease on day 19, and one animal never showed signs of disease.

In group 2, on day 12, one animal received a score of 2 in both hind limbs and another animal received a score of 3 in one hind limb and a score of 2 in the other hind limb. By day 15, all animals in the group were given a score of at least 2 in one hind limb, and 4/8 animals received scores of 4 in one or both hind limbs, indicating the onset of severe arthritic disease. By day 18, all animals had received a score of 4 in each hind limb, and in some instances, in the fore limbs as well.

In group 4, on day 14, 5/8 animals received a clinical score of at least 2 in one or more hind limbs. By day 18, all animals received clinical scores of 3 or greater in one or more hind limbs. On day 21, 3/8 animals received a clinical score of 4 in each hind limb, and 5/8 animals received a score of 4 in at least one hind limb.

In group 5, on day 13, 1/8 animals received a clinical score of 2 in one hind limb. On day 14, 2/8 animals had clinical scores of at least 2 in one or more hind limbs, with one animal receiving a score of 3 for each hind limb, and the other receiving a score of 2 for one hind limb. On days 18, 19 and 21, 6/8 animals received scores of 4 in each hind limb, and 2/8 animals received a score of 4 in one hind limb and a score of 3 in the other hind limb. Several animals additionally were given scores of 4 in one or both hind limbs on days 18, 19 and 21.

In group 6, on day 12, one animal received a score of 2 in one hind limb. On day 14, 1/8 animal had received a score of 4 in a hind limb, and 3/8 animals had received a score of 3 in one or more hind limbs. On day 18, 4/8 animals received scores of 4 in one or more hind limbs, but clinical manifestations improved on days 19 and 21, where only 2/8 animals received scores of 4 on one or more hind limbs. On day 21, 3/8 animals received scores of 3 on each hind limb, 2/8 animals received scores of 1 or 2 on both hind limbs, and 1/8 animals received a score of 2 on one hind limb and no score on the other hind limb.

In group 7, on day 15, 2/8 animals received a score of 2 on one hind limb. On day 18, 3/8 animals received scores of 2 or 3 on both hind limbs. On day 19, 4/8 animals received a score of 2 or 3 on both hind limbs. On day 21, 4/8 animals received a clinical score of 3 on both hind limbs, and 3/8 animals received a score of 1 on one or both hind limbs, and 1/8 animals received no clinical score.

Table 15D shows the average overall clinical score for animals in each treatment group. As expected, animals where CIA was not induced (group 1) showed no signs of disease over the 21-day period. In contrast, group 2, where CIA was induced and no treatment was provided (group 2), exhibited an increase in clinical scores starting on day 12, and plateauing with an average clinical score of 13 around day 18. As previously noted, dexamethasone treated animals (group 3) never exhibited any signs of disease. Animals receiving GSK620 (group 4) exhibited an increase in clinical scores starting at day 14, with scores plateauing at an average clinical score of 8 around day 18. On day 18, animals in group 4 exhibited an average clinical score that was significantly decreased (p<0.05) relative to group 2. Animals in group 5 exhibited a clinical score that progressed similar to other treatment groups, plateauing at an average score of 10 around day 18. Animals in groups 6 and 7 had average clinical scores that plateaued at 7 and 4, respectively. In particular, animals in group 7 exhibited an average clinical score that was significantly decreased (p<0.01) relative to animals in group 2 on days 18, 19 and 21.

Without being bound by any particular theory, the three different doses of Example 101 (3 mg/kg, group 5; 10 mg/kg, group 6; and 30 mg/kg, group 7) appeared to exhibit a dose-dependent effect in the amelioration of clinical symptoms associated with CIA.

Animals where CIA was not induced (group 1), as well as animals receiving vehicle (group 2), dexamethasone (group 3), GSK620 (group 4), and 3 mg/kg of Example 101 did not have diarrhea over the 21-day period (Table 10E). Two different animals receiving 10 mg/kg of Example 101 (group 6) had diarrhea for a single day over the 21-day period. Four different animals receiving 30 mg/kg of Example 101 (group 7) had diarrhea over the 21-day period. Of the animals in the group 7, one animal had diarrhea on two days, day 13 and day 18, and another animal had diarrhea on three days, day 16, day 19, and day 21. No animals in the study experienced diarrhea on successive days.

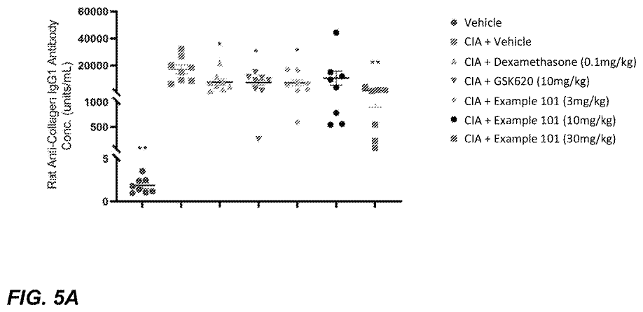

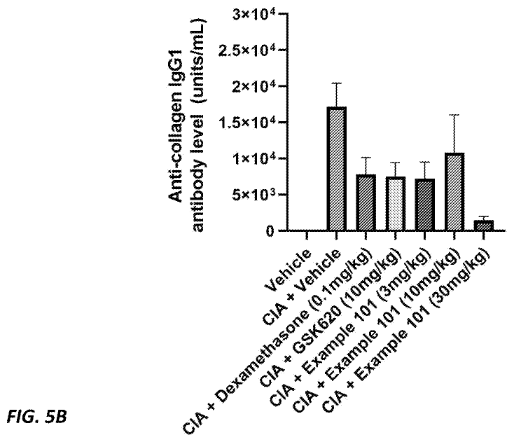

Without being bound by any particular theory, Example 101 appeared to function in a dose-dependent manner to limit and/or reduce the severity of CIA-related symptoms in animals, with the higher dosages of 10 mg/kg and 30 mg/kg proving most effective as shown by clinical scoring of CIA-related symptoms. As indicated by the dog PK studies 1 and 2 provided earlier (comparing a propylene glycol-based formulation and a HPβCD-based formulation), the slight reduction in tolerance may be, without being bound by any theory, due to the propylene glycol formulation rather than Example 101. Tables 16A and 16B and FIGS. 5A-5B illustrate levels of rat anti-collagen IgG1 antibodies in animals at day 21 of the study. Animals receiving Example 101 (1 mg/kg, 10 mg/kg) exhibited significantly decreased levels of rat anti-collagen IgG1 antibodies relative to animals where CIA was induced and no treatment was provided (p<0.05). Similarly, animals receiving the high dose of Example 101 (30 mg/kg) also exhibited significantly decreased levels of rat anti-collagen IgG1 antibodies relative to animals where CIA was induced and no treatment was provided (p<0.01)

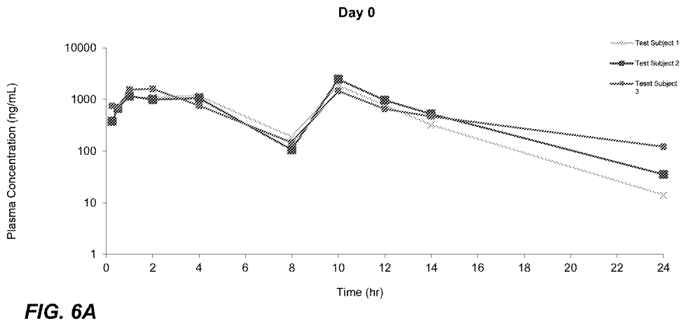

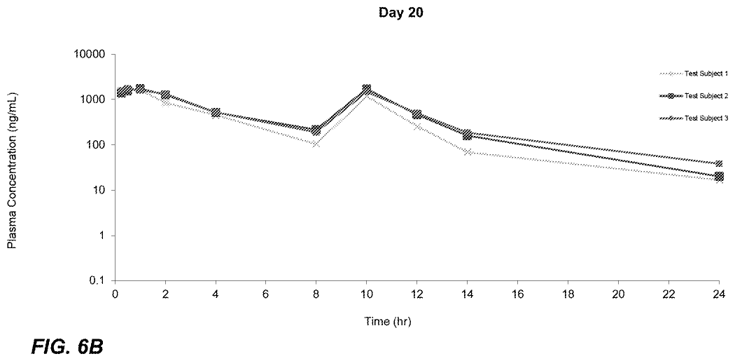

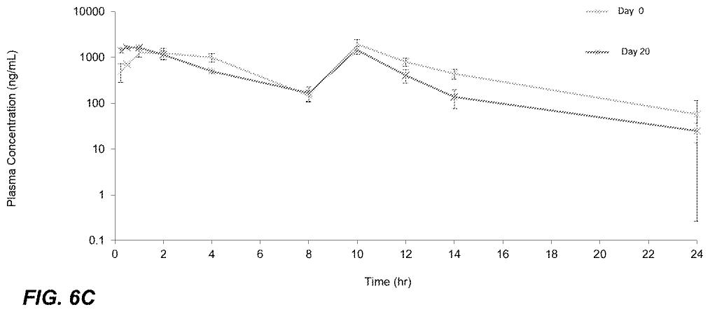

Tables 17A-17J and FIGS. 6A-6C depict the pharmacokinetic profile of Example 101 after oral administration in Lewis rats. Without being bound by any particular theory, day plasma concentration of Example 101 appeared to increase in a dose-dependent manner. Plasma concentration of Example 101 peaked shortly after administration of the first dose, and then gradually declined until the second daily dose was administered at 8 h, where plasma concentration of Example 101 increased and levels peaked at 10 h before decreasing over the remaining time points. The pharmacokinetic profile indicates that Example 101 may be therapeutically effective with a twice-daily unit dose. The free EC50 for GSK620 was 771 ng/mL, whereas the free EC50 for Example 101 was 207 ng/mL. A plasma concentration of a compound above the free EC50 for a significant period of time following an oral dose is an indication of an effective treatment window. Also, having a low free EC50 can allow for different unit treatment doses that can be therapeutically effective, including a low unit dose of a compound.

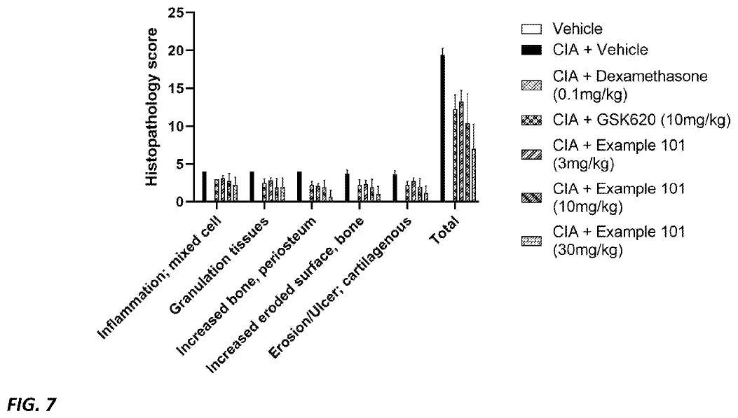

Results from the histopathological analysis are outlined in Table 18. Mean histopathologic scores for tissue samples from animals in Groups 1-7 are depicted in FIG. 7. Animals in the vehicle group are represented by the solid white bar, animals in the CIA+Vehicle group are represented by the solid black bar, animals in the CIA+Dexamethasone group are represented by the grey bar with white checkers, animals in the CIA+GSK620 group are represented by the grey bar with black checkers, animals in the CIA+Example 101 (3 mg/kg) group are represented by the grey bar with the white diagonal slash, animals in the CIA_Example 101 (10 mg/kg) group are represented by the grey bar with the black diagonal slash, and animals in the CIA+Example 101 (30 mg/kg) group are represented by the grey bar with the brick pattern.

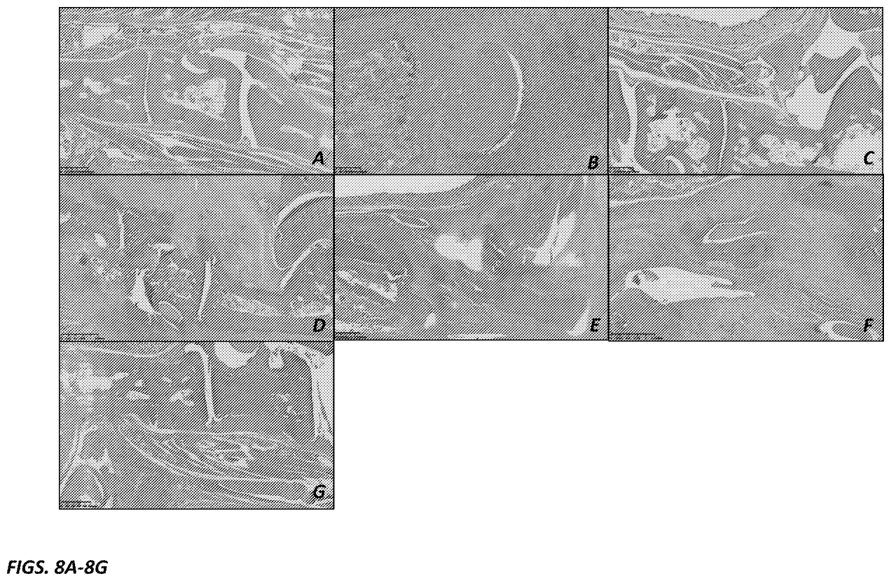

Representative images depicting different grades of histological measures are provided in FIGS. 8A-8G. FIG. 8A shows a representative sample from Group 1 (No CIA) depicting normal tissue having no histopathologic observations. FIG. 8B shows a representative sample from Group 2 (CIA) depicting marked mixed cell inflammation, marked granulation tissue, moderate increased eroded surface of bone, marked increased periosteal bone, and moderate erosion/ulcer in articular cartilage. FIG. 8C shows a representative sample from Group 3 (CIA+Dexamethasone) depicting tissue with no histopathological observations noted. FIG. 8D shows a representative sample from Group 4 (CIA+GSK620) depicting moderate mixed cell inflammation, mild granulation tissue, mild increased eroded surface of bone, mild increased periosteal bone, and mild erosion/ulcer in articular cartilage. FIG. 8E shows a representative sample from Group 5 (CIA+Example 101, 3 mg/kg) depicting moderate mixed cell inflammation, mild granulation tissue, moderate increased eroded surface of bone, moderate increased periosteal bone, and moderate erosion/ulcer in articular cartilage. FIG. 8F shows a representative sample from Group 6 (CIA+Example 101, 10 mg/kg) depicting moderate mixed cell inflammation, mild granulation tissue, mild increased eroded surface of bone, minimal increased periosteal bone, and mild erosion/ulcer in articular cartilage. FIG. 8G shows a representative sample from Group 7 (CIA+Example 101, 30 mg/kg), depicting mild mixed cell inflammation and mild granulation tissue.

No pathologic observations were noted after microscopic evaluation for samples from animals in the vehicle-treated (sham) group (Group 1).

Pathologic observations were noted after microscopic evaluation for all samples from animals in the CIA-induced group (Group 2). Observations included marked mixed cell inflammation, marked granulation tissue (pannus), marked increased eroded surface of bone (bone resorption), moderate to marked increased periosteal bone (periosteal bone formation), and moderate to marked erosion/ulcer in articular cartilage.

No pathologic observations were noted after microscopic evaluation for samples from animals in the dexamethasone treatment group (Group 3).

Pathologic observations were noted after microscopic evaluation for all samples from animals in the GSK620 treatment group (Group 4), albeit at a lower level relative to samples from animals in the CIA-induced group (Group 2). Observations included moderate mixed cell inflammation, mild to moderate granulation tissue (pannus), minimal to moderate increased eroded surface of bone (bone resorption), mild to moderate increased periosteal bone (periosteal bone formation), and mild to moderate erosion/ulcer in the articular cartilage.

Pathologic observations were noted after microscopic evaluation of samples from animals in the Example 101 treatment groups (Groups 5-7), albeit at lower levels relative to samples from animals in the CIA-induced group (Group 2) in a dose-dependent manner.

All samples from animals receiving Example 101 (3 mg/kg; Group 5) had pathologic observations. Observations included moderate to marked mixed cell inflammation, mild to moderate granulation tissue (pannus), mild to moderate increased eroded surface of bone (bone resorption), mild to moderate increased periosteal bone (periosteal bone formation), and mild to moderate articular cartilage erosion or ulcers.

All samples from animals receiving Example 101 (10 mg/kg; Group 6) had pathologic observations. Observations included moderate mixed cell inflammation, moderate granulation tissue (pannus), moderate increased eroded surface of bone (bone resorption), moderate increased periosteal bone (periosteal bone formation), and moderate erosion in the articular cartilages.

One out of eight samples collected from animals receiving Example 101 (30 mg/kg; Group 7) had no recorded pathological observations. The remaining seven samples exhibited moderate mixed cell inflammation, moderate granulation tissue (pannus), mild increased eroded surface of bone (bone resorption), mild increased periosteal bone (periosteal bone formation), and mild erosion in the articular cartilages.

### Tables—Part A: Body Weight, Paw Volumes, and Clinical Scores

TABLE 13A
Animal body weight (g)

| Group | Animal # | Day 0 | Day 1 | Day 2 | Day 3 | Day 4 | Day 5 | Day 6 | Day 7 |
|---|---|---|---|---|---|---|---|---|---|
| 1 (No CIA + | 1 | 165 | 165 | 167 | 170 | 173 | 173 | 178 | 183 |
| Vehicle) | 2 | 156 | 161 | 162 | 166 | 170 | 173 | 173 | 172 |
| | 3 | 170 | 172 | 175 | 175 | 183 | 183 | 185 | 189 |
| | 4 | 168 | 165 | 168 | 169 | 171 | 179 | 179 | 179 |
| | 5 | 175 | 177 | 177 | 180 | 185 | 185 | 194 | 185 |
| | 6 | 166 | 165 | 163 | 167 | 170 | 176 | 177 | 178 |
| | 7 | 167 | 170 | 173 | 173 | 171 | 171 | 176 | 176 |
| | 8 | 161 | 164 | 166 | 161 | 165 | 169 | 170 | 173 |
| 2 (CIA + | 1 | 165 | 169 | 171 | 176 | 172 | 180 | 184 | 183 |
| Vehicle) | 2 | 163 | 171 | 170 | 170 | 173 | 175 | 180 | 183 |
| | 3 | 164 | 163 | 166 | 164 | 170 | 173 | 178 | 174 |
| | 4 | 175 | 179 | 177 | 185 | 182 | 189 | 187 | 190 |
| | 5 | 152 | 156 | 155 | 158 | 161 | 162 | 170 | 172 |
| | 6 | 160 | 160 | 157 | 160 | 161 | 170 | 172 | 173 |
| | 7 | 153 | 155 | 152 | 155 | 158 | 156 | 164 | 162 |
| | 8 | 155 | 157 | 161 | 165 | 166 | 167 | 173 | 174 |
| 3 (CIA + | 1 | 151 | 152 | 150 | 148 | 147 | 147 | 147 | 150 |
| Dexamethasone) | 2 | 162 | 150 | 151 | 144 | 147 | 149 | 152 | 153 |
| | 3 | 174 | 171 | 176 | 175 | 170 | 165 | 171 | 178 |
| | 4 | 169 | 165 | 166 | 164 | 160 | 164 | 161 | 162 |
| | 5 | 166 | 164 | 161 | 162 | 156 | 158 | 155 | 160 |
| | 6 | 161 | 151 | 148 | 145 | 148 | 150 | 149 | 145 |
| | 7 | 172 | 167 | 165 | 164 | 165 | 166 | 165 | 162 |
| | 8 | 160 | 158 | 156 | 156 | 157 | 151 | 157 | 161 |
| 4 (CIA + | 1 | 157 | 160 | 162 | 163 | 159 | 167 | 169 | 170 |
| GSK620) | 2 | 168 | 173 | 167 | 167 | 172 | 177 | 171 | 175 |
| | 3 | 165 | 171 | 168 | 168 | 172 | 172 | 172 | 170 |
| | 4 | 162 | 165 | 171 | 173 | 173 | 177 | 171 | 171 |
| | 5 | 170 | 175 | 173 | 172 | 171 | 180 | 177 | 180 |
| | 6 | 161 | 167 | 164 | 166 | 165 | 171 | 171 | 172 |
| | 7 | 173 | 178 | 179 | 181 | 179 | 181 | 186 | 183 |
| | 8 | 166 | 172 | 170 | 173 | 171 | 176 | 177 | 176 |
| 5 (CIA + Example | 1 | 161 | 159 | 162 | 160 | 158 | 163 | 167 | 168 |
| 101 3 mg/kg) | 2 | 159 | 153 | 149 | 156 | 154 | 155 | 156 | 160 |
| | 3 | 171 | 170 | 167 | 173 | 173 | 177 | 176 | 177 |
| | 4 | 177 | 182 | 178 | 175 | 172 | 177 | 180 | 182 |
| | 5 | 155 | 156 | 159 | 161 | 161 | 158 | 160 | 167 |
| | 6 | 166 | 162 | 164 | 165 | 170 | 171 | 175 | 175 |
| | 7 | 165 | 163 | 169 | 172 | 174 | 173 | 176 | 180 |
| | 8 | 165 | 168 | 169 | 170 | 169 | 169 | 174 | 173 |
| 6 (CIA + Example | 1 | 171 | 168 | 168 | 173 | 172 | 178 | 177 | 180 |
| 101 10 mg/kg) | 2 | 156 | 149 | 143 | 146 | 149 | 146 | 142 | 147 |
| | 3 | 170 | 168 | 163 | 171 | 171 | 175 | 167 | 172 |
| | 4 | 162 | 162 | 163 | 167 | 168 | 165 | 167 | 167 |
| | 5 | 163 | 152 | 160 | 165 | 162 | 158 | 165 | 162 |
| | 6 | 156 | 157 | 161 | 163 | 159 | 166 | 170 | 172 |
| | 7 | 160 | 168 | 169 | 171 | 172 | 170 | 176 | 175 |
| | 8 | 161 | 156 | 155 | 155 | 159 | 160 | 158 | 153 |
| 7 (CIA + Example | 1 | 155 | 160 | 158 | 158 | 164 | 163 | 168 | 167 |
| 101 30 mg/kg) | 2 | 172 | 170 | 170 | 166 | 165 | 172 | 164 | 165 |
| | 3 | 165 | 164 | 156 | 156 | 163 | 162 | 163 | 161 |
| | 4 | 164 | 159 | 161 | 162 | 166 | 165 | 166 | 165 |
| | 5 | 164 | 159 | 157 | 159 | 161 | 159 | 157 | 155 |
| | 6 | 166 | 168 | 168 | 165 | 167 | 169 | 163 | 164 |
| | 7 | 154 | 154 | 161 | 160 | 160 | 158 | 157 | 162 |
| | 8 | 156 | 151 | 151 | 152 | 155 | 159 | 157 | 159 |

| Group | Animal # | Day 8 | Day 9 | Day 10 | Day 11 | Day 12 | Day 13 | Day 14 |
|---|---|---|---|---|---|---|---|---|
| 1 (No CIA + | 1 | 178 | 180 | 184 | 187 | 192 | 192 | 191 |
| Vehicle) | 2 | 175 | 181 | 180 | 177 | 187 | 185 | 185 |
| | 3 | 192 | 190 | 192 | 199 | 200 | 204 | 204 |
| | 4 | 181 | 186 | 187 | 188 | 183 | 189 | 191 |
| | 5 | 192 | 196 | 194 | 195 | 202 | 204 | 205 |
| | 6 | 171 | 180 | 182 | 183 | 183 | 183 | 183 |
| | 7 | 176 | 180 | 183 | 184 | 185 | 185 | 185 |
| | 8 | 172 | 176 | 177 | 180 | 180 | 181 | 182 |
| 2 (CIA + | 1 | 180 | 190 | 188 | 190 | 192 | 200 | 197 |
| Vehicle) | 2 | 182 | 184 | 183 | 189 | 190 | 193 | 190 |
| | 3 | 174 | 182 | 183 | 180 | 187 | 192 | 191 |
| | 4 | 193 | 197 | 195 | 193 | 202 | 203 | 203 |
| | 5 | 172 | 172 | 174 | 174 | 177 | 180 | 174 |
| | 6 | 170 | 172 | 173 | 176 | 173 | 174 | 168 |
| | 7 | 163 | 169 | 173 | 175 | 173 | 177 | 180 |
| | 8 | 173 | 171 | 175 | 180 | 181 | 174 | 176 |
| 3 (CIA + | 1 | 147 | 153 | 154 | 155 | 153 | 151 | 157 |
| Dexamethasone) | 2 | 143 | 145 | 152 | 150 | 152 | 150 | 147 |
| | 3 | 174 | 176 | 176 | 183 | 185 | 186 | 181 |
| | 4 | 160 | 166 | 164 | 167 | 165 | 162 | 166 |
| | 5 | 161 | 162 | 159 | 157 | 162 | 167 | 163 |
| | 6 | 152 | 158 | 150 | 150 | 147 | 148 | 150 |
| | 7 | 167 | 172 | 175 | 173 | 170 | 176 | 175 |
| | 8 | 160 | 159 | 155 | 164 | 166 | 164 | 160 |
| 4 (CIA + | 1 | 168 | 171 | 165 | 174 | 177 | 176 | 170 |
| GSK620) | 2 | 176 | 177 | 177 | 182 | 185 | 182 | 176 |
| | 3 | 178 | 181 | 181 | 181 | 184 | 182 | 180 |
| | 4 | 177 | 178 | 180 | 178 | 179 | 179 | 177 |
| | 5 | 184 | 183 | 189 | 189 | 190 | 194 | 191 |
| | 6 | 171 | 174 | 178 | 177 | 181 | 175 | 176 |
| | 7 | 183 | 186 | 186 | 186 | 186 | 185 | 184 |
| | 8 | 176 | 182 | 180 | 183 | 183 | 187 | 182 |
| 5 (CIA + Example | 1 | 167 | 170 | 172 | 172 | 171 | 169 | 171 |
| 101 3 mg/kg) | 2 | 160 | 164 | 163 | 165 | 163 | 161 | 158 |
| | 3 | 181 | 182 | 181 | 179 | 183 | 180 | 180 |
| | 4 | 182 | 186 | 184 | 193 | 192 | 192 | 188 |
| | 5 | 163 | 163 | 167 | 167 | 173 | 176 | 176 |
| | 6 | 177 | 179 | 182 | 181 | 182 | 180 | 177 |
| | 7 | 176 | 175 | 181 | 185 | 185 | 182 | 179 |
| | 8 | 175 | 174 | 179 | 180 | 178 | 181 | 183 |
| 6 (CIA + Example | 1 | 181 | 181 | 182 | 186 | 183 | 177 | 179 |
| 101 10 mg/kg) | 2 | 147 | 153 | 151 | 148 | 146 | 150 | 148 |
| | 3 | 174 | 176 | 173 | 169 | 166 | 164 | 165 |
| | 4 | 173 | 168 | 172 | 175 | 172 | 163 | 165 |
| | 5 | 159 | 166 | 167 | 168 | 166 | 171 | 169 |
| | 6 | 164 | 173 | 174 | 174 | 173 | 175 | 175 |
| | 7 | 180 | 182 | 182 | 183 | 189 | 189 | 187 |
| | 8 | 158 | 163 | 161 | 162 | 158 | 160 | 163 |
| 7 (CIA + Example | 1 | 166 | 170 | 169 | 170 | 169 | 168 | 171 |
| 101 30 mg/kg) | 2 | 169 | 171 | 171 | 168 | 170 | 167 | 167 |
| | 3 | 164 | 167 | 165 | 166 | 161 | 161 | 163 |
| | 4 | 163 | 166 | 164 | 162 | 160 | 163 | 167 |
| | 5 | 155 | 161 | 158 | 157 | 153 | 152 | 157 |
| | 6 | 165 | 169 | 171 | 171 | 168 | 170 | 169 |
| | 7 | 164 | 164 | 162 | 164 | 161 | 156 | 158 |
| | 8 | 164 | 163 | 164 | 165 | 161 | 160 | 161 |
| Group | Animal # | Day 15 | Day 16 | Day 17 | Day 18 | Day 19 | Day 20 | Day 21 |
| 1 (No CIA + | 1 | 192 | 191 | 185 | 192 | 197 | 196 | 196 |
| Vehicle) | 2 | 183 | 186 | 185 | 189 | 185 | 186 | 190 |
| | 3 | 206 | 205 | 209 | 205 | 214 | 216 | 217 |
| | 4 | 191 | 189 | 190 | 191 | 189 | 191 | 192 |
| | 5 | 201 | 209 | 215 | 214 | 210 | 215 | 219 |
| | 6 | 186 | 189 | 193 | 189 | 194 | 197 | 198 |
| | 7 | 190 | 190 | 190 | 184 | 190 | 192 | 195 |
| | 8 | 185 | 183 | 187 | 191 | 191 | 192 | 189 |
| 2 (CIA + | 1 | 194 | 189 | 190 | 186 | 181 | 175 | 174 |
| Vehicle) | 2 | 185 | 190 | 190 | 186 | 184 | 185 | 184 |
| | 3 | 183 | 181 | 181 | 175 | 172 | 170 | 168 |
| | 4 | 199 | 200 | 197 | 195 | 193 | 189 | 191 |
| | 5 | 168 | 167 | 165 | 168 | 165 | 162 | 159 |
| | 6 | 172 | 180 | 174 | 173 | 170 | 173 | 171 |
| | 7 | 176 | 175 | 175 | 175 | 174 | 169 | 166 |
| | 8 | 174 | 174 | 170 | 166 | 166 | 167 | 169 |
| 3 (CIA + | 1 | 166 | 157 | 160 | 157 | 162 | 163 | 164 |
| Dexamethasone) | 2 | 157 | 153 | 155 | 152 | 147 | 156 | 156 |
| | 3 | 178 | 187 | 188 | 189 | 192 | 184 | 187 |
| | 4 | 170 | 168 | 165 | 165 | 170 | 173 | 169 |
| | 5 | 164 | 159 | 167 | 166 | 168 | 167 | 161 |
| | 6 | 150 | 147 | 150 | 152 | 152 | 154 | 152 |
| | 7 | 176 | 174 | 170 | 179 | 182 | 180 | 177 |
| | 8 | 159 | 163 | 168 | 169 | 163 | 160 | 166 |
| 4 (CIA + | 1 | 172 | 170 | 168 | 164 | 161 | 163 | 162 |
| GSK620) | 2 | 168 | 168 | 168 | 166 | 165 | 163 | 163 |
| | 3 | 184 | 186 | 183 | 182 | 174 | 168 | 170 |
| | 4 | 173 | 171 | 171 | 168 | 170 | 164 | 165 |
| | 5 | 194 | 194 | 192 | 184 | 183 | 186 | 182 |
| | 6 | 174 | 171 | 166 | 161 | 155 | 161 | 160 |
| | 7 | 179 | 173 | 172 | 169 | 165 | 170 | 165 |
| | 8 | 180 | 172 | 169 | 170 | 170 | 169 | 165 |
| 5 (CIA + Example | 1 | 164 | 169 | 167 | 166 | 166 | 164 | 166 |
| 101 3 mg/kg) | 2 | 156 | 150 | 152 | 152 | 150 | 152 | 153 |
| | 3 | 181 | 172 | 173 | 175 | 173 | 173 | 176 |
| | 4 | 185 | 181 | 182 | 182 | 179 | 179 | 181 |
| | 5 | 169 | 170 | 170 | 167 | 161 | 160 | 162 |
| | 6 | 174 | 172 | 169 | 166 | 161 | 167 | 167 |
| | 7 | 182 | 178 | 175 | 171 | 170 | 169 | 168 |
| | 8 | 176 | 178 | 175 | 171 | 171 | 170 | 169 |
| 6 (CIA + Example | 1 | 173 | 171 | 173 | 171 | 170 | 167 | 166 |
| 101 10 mg/kg) | 2 | 148 | 148 | 145 | 144 | 146 | 144 | 142 |
| | 3 | 165 | 162 | 162 | 160 | 160 | 163 | 161 |
| | 4 | 161 | 161 | 163 | 160 | 160 | 161 | 161 |
| | 5 | 170 | 165 | 161 | 159 | 159 | 162 | 158 |
| | 6 | 176 | 168 | 167 | 168 | 165 | 167 | 165 |
| | 7 | 183 | 187 | 192 | 188 | 182 | 181 | 18 |
| | 8 | 165 | 165 | 165 | 166 | 166 | 170 | 171 |
| 7 (CIA + Example | 1 | 167 | 168 | 169 | 173 | 175 | 174 | 173 |
| 101 30 mg/kg) | 2 | 172 | 169 | 166 | 171 | 168 | 165 | 163 |
| | 3 | 165 | 161 | 163 | 162 | 163 | 158 | 158 |
| | 4 | 170 | 172 | 168 | 171 | 173 | 174 | 174 |
| | 5 | 157 | 159 | 159 | 159 | 159 | 160 | 161 |
| | 6 | 174 | 171 | 170 | 169 | 164 | 160 | 161 |
| | 7 | 160 | 155 | 156 | 152 | 151 | 151 | 150 |
| | 8 | 161 | 162 | 162 | 159 | 158 | 159 | 161 |

TABLE 13B
Animal body weight percent change (%)

| Group | Animal # | Day 0 | Day 1 | Day 2 | Day 3 | Day 4 | Day 5 | Day 6 | Day 7 |
|---|---|---|---|---|---|---|---|---|---|
| 1 (No CIA + | 1 | 0.00 | 0.00 | 1.21 | 3.03 | 4.85 | 4.85 | 7.88 | 10.91 |
| Vehicle) | 2 | 0.00 | 3.21 | 3.85 | 6.41 | 8.97 | 10.90 | 10.90 | 10.26 |
| | 3 | 0.00 | 1.18 | 2.94 | 2.94 | 7.65 | 7.65 | 8.82 | 11.18 |
| | 4 | 0.00 | −1.79 | 0.00 | 0.60 | 1.79 | 6.55 | 6.55 | 6.55 |
| | 5 | 0.00 | 1.14 | 1.14 | 2.86 | 5.71 | 5.71 | 10.86 | 5.71 |
| | 6 | 0.00 | −0.60 | −1.81 | 0.60 | 2.41 | 6.02 | 6.63 | 7.23 |
| | 7 | 0.00 | 1.80 | 3.59 | 3.59 | 2.40 | 2.40 | 5.39 | 5.39 |
| | 8 | 0.00 | 1.86 | 3.11 | 0.00 | 2.48 | 4.97 | 5.59 | 7.45 |
| 2 (CIA + | 1 | 0.00 | 2.42 | 3.64 | 6.67 | 4.24 | 9.09 | 11.52 | 10.91 |
| Vehicle) | 2 | 0.00 | 4.91 | 4.29 | 4.29 | 6.13 | 7.36 | 10.43 | 12.27 |
| | 3 | 0.00 | −0.61 | 1.22 | 0.00 | 3.66 | 5.49 | 8.54 | 6.10 |
| | 4 | 0.00 | 2.29 | 1.14 | 5.71 | 4.00 | 8.00 | 6.86 | 8.57 |
| | 5 | 0.00 | 2.63 | 1.97 | 3.95 | 5.92 | 6.58 | 11.84 | 13.16 |
| | 6 | 0.00 | 0.00 | −1.88 | 0.00 | 0.63 | 6.25 | 7.50 | 8.13 |
| | 7 | 0.00 | 1.31 | −0.65 | 1.31 | 3.27 | 1.96 | 7.19 | 5.88 |
| | 8 | 0.00 | 1.29 | 3.87 | 6.45 | 7.10 | 7.74 | 11.61 | 12.26 |
| 3 (CIA + | 1 | 0.00 | 0.66 | −0.66 | −1.99 | −2.65 | −2.65 | −2.65 | −0.66 |
| Dexamethasone) | 2 | 0.00 | −7.41 | −6.79 | −11.11 | −9.26 | −8.02 | −6.17 | −5.56 |
| | 3 | 0.00 | −1.72 | 1.15 | 0.57 | −2.30 | −5.17 | −1.72 | 2.30 |
| | 4 | 0.00 | −2.37 | −1.78 | −2.96 | −5.33 | −2.96 | −4.73 | −4.14 |
| | 5 | 0.00 | −1.20 | −3.01 | −2.41 | −6.02 | −4.82 | −6.63 | −3.61 |
| | 6 | 0.00 | −6.21 | −8.07 | −9.94 | −8.07 | −6.83 | −7.45 | −9.94 |
| | 7 | 0.00 | −2.91 | −4.07 | −4.65 | −4.07 | −3.49 | −4.07 | −5.81 |
| | 8 | 0.00 | −1.25 | −2.50 | −2.50 | −1.88 | −5.63 | −1.88 | 0.63 |
| 4 (CIA + | 1 | 0.00 | 1.91 | 3.18 | 3.82 | 1.27 | 6.37 | 7.64 | 8.28 |
| GSK620) | 2 | 0.00 | 2.98 | −0.60 | −0.60 | 2.38 | 5.36 | 1.79 | 4.17 |
| | 3 | 0.00 | 3.64 | 1.82 | 1.82 | 4.24 | 4.24 | 4.24 | 3.03 |
| | 4 | 0.00 | 1.85 | 5.56 | 6.79 | 6.79 | 9.26 | 5.56 | 5.56 |
| | 5 | 0.00 | 2.94 | 1.76 | 1.18 | 0.59 | 5.88 | 4.12 | 5.88 |
| | 6 | 0.00 | 3.73 | 1.86 | 3.11 | 2.48 | 6.21 | 6.21 | 6.83 |
| | 7 | 0.00 | 2.89 | 3.47 | 4.62 | 3.47 | 4.62 | 7.51 | 5.78 |
| | 8 | 0.00 | 3.61 | 2.41 | 4.22 | 3.01 | 6.02 | 6.63 | 6.02 |
| 5 (CIA + Example | 1 | 0.00 | −1.24 | 0.62 | −0.62 | −1.86 | 1.24 | 3.73 | 4.35 |
| 101 3 mg/kg) | 2 | 0.00 | −3.77 | −6.29 | −1.89 | −3.14 | −2.52 | −1.89 | 0.63 |
| | 3 | 0.00 | −0.58 | −2.34 | 1.17 | 1.17 | 3.51 | 2.92 | 3.51 |
| | 4 | 0.00 | 2.82 | 0.56 | −1.13 | −2.82 | 0.00 | 1.69 | 2.82 |
| | 5 | 0.00 | 0.65 | 2.58 | 3.87 | 3.87 | 1.94 | 3.23 | 7.74 |
| | 6 | 0.00 | −2.41 | −1.20 | −0.60 | 2.41 | 3.01 | 5.42 | 5.42 |
| | 7 | 0.00 | −1.21 | 2.42 | 4.24 | 5.45 | 4.85 | 6.67 | 9.09 |
| | 8 | 0.00 | 1.82 | 2.42 | 3.03 | 2.42 | 2.42 | 5.45 | 4.85 |
| 6 (CIA + Example | 1 | 0.00 | −1.75 | −1.75 | 1.17 | 0.58 | 4.09 | 3.51 | 5.26 |
| 101 10 mg/kg) | 2 | 0.00 | −4.49 | −8.33 | −6.41 | −4.49 | −6.41 | −8.97 | −5.77 |
| | 3 | 0.00 | −1.18 | −4.12 | 0.59 | 0.59 | 2.94 | −1.76 | 1.18 |
| | 4 | 0.00 | 0.00 | 0.62 | 3.09 | 3.70 | 1.85 | 3.09 | 3.09 |
| | 5 | 0.00 | −6.75 | −1.84 | 1.23 | −0.61 | −3.07 | 1.23 | −0.61 |
| | 6 | 0.00 | 0.64 | 3.21 | 4.49 | 1.92 | 6.41 | 8.97 | 10.26 |
| | 7 | 0.00 | 5.00 | 5.63 | 6.88 | 7.50 | 6.25 | 10.00 | 9.38 |
| | 8 | 0.00 | −3.11 | −3.73 | −3.73 | −1.24 | −0.62 | −1.86 | −4.97 |
| 7 (CIA + Example | 1 | 0.00 | 3.23 | 1.94 | 1.94 | 5.81 | 5.16 | 8.39 | 7.74 |
| 101 30 mg/kg) | 2 | 0.00 | −1.16 | −1.16 | −3.49 | −4.07 | 0.00 | −4.65 | −4.07 |
| | 3 | 0.00 | −0.61 | −5.45 | −5.45 | −1.21 | −1.82 | −1.21 | −2.42 |
| | 4 | 0.00 | −3.05 | −1.83 | −1.22 | 1.22 | 0.61 | 1.22 | 0.61 |
| | 5 | 0.00 | −3.05 | −4.27 | −3.05 | −1.83 | −3.05 | −4.27 | −5.49 |
| | 6 | 0.00 | 1.20 | 1.20 | −0.60 | 0.60 | 1.81 | −1.81 | −1.20 |
| | 7 | 0.00 | 0.00 | 4.55 | 3.90 | 3.90 | 2.60 | 1.95 | 5.19 |
| | 8 | 0.00 | −3.21 | −3.21 | −2.56 | −0.64 | 1.92 | 0.64 | 1.92 |

| Group | Animal # | Day 8 | Day 9 | Day 10 | Day 11 | Day 12 | Day 13 | Day 14 |
|---|---|---|---|---|---|---|---|---|
| 1 (No CIA + | 1 | 7.88 | 9.09 | 11.52 | 13.33 | 16.36 | 16.36 | 15.76 |
| Vehicle) | 2 | 12.18 | 16.03 | 15.38 | 13.46 | 19.87 | 18.59 | 18.59 |
| | 3 | 12.94 | 11.76 | 12.94 | 17.06 | 17.65 | 20.00 | 20.00 |
| | 4 | 7.74 | 10.71 | 11.31 | 11.90 | 8.93 | 12.50 | 13.69 |
| | 5 | 9.71 | 12.00 | 10.86 | 11.43 | 15.43 | 16.57 | 17.14 |
| | 6 | 3.01 | 8.43 | 9.64 | 10.24 | 10.24 | 10.24 | 10.24 |
| | 7 | 5.39 | 7.78 | 9.58 | 10.18 | 10.78 | 10.78 | 10.78 |
| | 8 | 6.83 | 9.32 | 9.94 | 11.80 | 11.80 | 12.42 | 13.04 |
| 2 (CIA + | 1 | 9.09 | 15.15 | 13.94 | 15.15 | 16.36 | 21.21 | 19.39 |
| Vehicle) | 2 | 11.66 | 12.88 | 12.27 | 15.95 | 16.56 | 18.40 | 16.56 |
| | 3 | 6.10 | 10.98 | 11.59 | 9.76 | 14.02 | 17.07 | 16.46 |
| | 4 | 10.29 | 12.57 | 11.43 | 10.29 | 15.43 | 16.00 | 16.00 |
| | 5 | 13.16 | 13.16 | 14.47 | 14.47 | 16.45 | 18.42 | 14.47 |
| | 6 | 6.25 | 7.50 | 8.13 | 10.00 | 8.13 | 8.75 | 5.00 |
| | 7 | 6.54 | 10.46 | 13.07 | 14.38 | 13.07 | 15.69 | 17.65 |
| | 8 | 11.61 | 10.32 | 12.90 | 16.13 | 16.77 | 12.26 | 13.55 |
| 3 (CIA + | 1 | −2.65 | 1.32 | 1.99 | 2.65 | 1.32 | 0.00 | 3.97 |
| Dexamethasone) | 2 | −11.73 | −10.49 | −6.17 | −7.41 | −6.17 | −7.41 | −9.26 |
| | 3 | 0.00 | 1.15 | 1.15 | 5.17 | 6.32 | 6.90 | 4.02 |
| | 4 | −5.33 | −1.78 | −2.96 | −1.18 | −2.37 | −4.14 | −1.78 |
| | 5 | −3.01 | −2.41 | −4.22 | −5.42 | −2.41 | 0.60 | −1.81 |
| | 6 | −5.59 | −1.86 | −6.83 | −6.83 | −8.70 | −8.07 | −6.83 |
| | 7 | −2.91 | 0.00 | 1.74 | 0.58 | −1.16 | 2.33 | 1.74 |
| | 8 | 0.00 | −0.63 | −3.13 | 2.50 | 3.75 | 2.50 | 0.00 |
| 4 (CIA + | 1 | 7.01 | 8.92 | 5.10 | 10.83 | 12.74 | 12.10 | 8.28 |
| GSK620) | 2 | 4.76 | 5.36 | 5.36 | 8.33 | 10.12 | 8.33 | 4.76 |
| | 3 | 7.88 | 9.70 | 9.70 | 9.70 | 11.52 | 10.30 | 9.09 |
| | 4 | 9.26 | 9.88 | 11.11 | 9.88 | 10.49 | 10.49 | 9.26 |
| | 5 | 8.24 | 7.65 | 11.18 | 11.18 | 11.76 | 14.12 | 12.35 |
| | 6 | 6.21 | 8.07 | 10.56 | 9.94 | 12.42 | 8.70 | 9.32 |
| | 7 | 5.78 | 7.51 | 7.51 | 7.51 | 7.51 | 6.94 | 6.36 |
| | 8 | 6.02 | 9.64 | 8.43 | 10.24 | 10.24 | 12.65 | 9.64 |
| 5 (CIA + Example | 1 | 3.73 | 5.59 | 6.83 | 6.83 | 6.21 | 4.97 | 6.21 |
| 101 3 mg/kg) | 2 | 0.63 | 3.14 | 2.52 | 3.77 | 2.52 | 1.26 | −0.63 |
| | 3 | 5.85 | 6.43 | 5.85 | 4.68 | 7.02 | 5.26 | 5.26 |
| | 4 | 2.82 | 5.08 | 3.95 | 9.04 | 8.47 | 8.47 | 6.21 |
| | 5 | 5.16 | 5.16 | 7.74 | 7.74 | 11.61 | 13.55 | 13.55 |
| | 6 | 6.63 | 7.83 | 9.64 | 9.04 | 9.64 | 8.43 | 6.63 |
| | 7 | 6.67 | 6.06 | 9.70 | 12.12 | 12.12 | 10.30 | 8.48 |
| | 8 | 6.06 | 5.45 | 8.48 | 9.09 | 7.88 | 9.70 | 10.91 |
| 6 (CIA + Example | 1 | 5.85 | 5.85 | 6.43 | 8.77 | 7.02 | 3.51 | 4.68 |
| 101 10 mg/kg) | 2 | −5.77 | −1.92 | −3.21 | −5.13 | −6.41 | −3.85 | −5.13 |
| | 3 | 2.35 | 3.53 | 1.76 | −0.59 | −2.35 | −3.53 | −2.94 |
| | 4 | 6.79 | 3.70 | 6.17 | 8.02 | 6.17 | 0.62 | 1.85 |
| | 5 | −2.45 | 1.84 | 2.45 | 3.07 | 1.84 | 4.91 | 3.68 |
| | 6 | 5.13 | 10.90 | 11.54 | 11.54 | 10.90 | 12.18 | 12.18 |
| | 7 | 12.50 | 13.75 | 13.75 | 14.38 | 18.13 | 18.13 | 16.88 |
| | 8 | −1.86 | 1.24 | 0.00 | 0.62 | −1.86 | −0.62 | 1.24 |
| 7 (CIA + Example | 1 | 7.10 | 9.68 | 9.03 | 9.68 | 9.03 | 8.39 | 10.32 |
| 101 30 mg/kg) | 2 | −1.74 | −0.58 | −0.58 | −2.33 | −1.16 | −2.91 | −2.91 |
| | 3 | −0.61 | 1.21 | 0.00 | 0.61 | −2.42 | −2.42 | −1.21 |
| | 4 | −0.61 | 1.22 | 0.00 | −1.22 | −2.44 | −0.61 | 1.83 |
| | 5 | −5.49 | −1.83 | −3.66 | −4.27 | −6.71 | −7.32 | −4.27 |
| | 6 | −0.60 | 1.81 | 3.01 | 3.01 | 1.20 | 2.41 | 1.81 |
| | 7 | 6.49 | 6.49 | 5.19 | 6.49 | 4.55 | 1.30 | 2.60 |
| | 8 | 5.13 | 4.49 | 5.13 | 5.77 | 3.21 | 2.56 | 3.21 |
| Group | Animal # | Day 15 | Day 16 | Day 17 | Day 18 | Day 19 | Day 20 | Day 21 |
| 1 (No CIA + | 1 | 16.36 | 15.76 | 12.12 | 16.36 | 19.39 | 18.79 | 18.79 |
| Vehicle) | 2 | 17.31 | 19.23 | 18.59 | 21.15 | 18.59 | 19.23 | 21.79 |
| | 3 | 21.18 | 20.59 | 22.94 | 20.59 | 25.88 | 27.06 | 27.65 |
| | 4 | 13.69 | 12.50 | 13.10 | 13.69 | 12.50 | 13.69 | 14.29 |
| | 5 | 14.86 | 19.43 | 22.86 | 22.29 | 20.00 | 22.86 | 25.14 |
| | 6 | 12.05 | 13.86 | 16.27 | 13.86 | 16.87 | 18.67 | 19.28 |
| | 7 | 13.77 | 13.77 | 13.77 | 10.18 | 13.77 | 14.97 | 16.77 |
| | 8 | 14.91 | 13.66 | 16.15 | 18.63 | 18.63 | 19.25 | 17.39 |
| 2 (CIA + | 1 | 17.58 | 14.55 | 15.15 | 12.73 | 9.70 | 6.06 | 5.45 |
| Vehicle) | 2 | 13.50 | 16.56 | 16.56 | 14.11 | 12.88 | 13.50 | 12.88 |
| | 3 | 11.59 | 10.37 | 10.37 | 6.71 | 4.88 | 3.66 | 2.44 |
| | 4 | 13.71 | 14.29 | 12.57 | 11.43 | 10.29 | 8.00 | 9.14 |
| | 5 | 10.53 | 9.87 | 8.55 | 10.53 | 8.55 | 6.58 | 4.61 |
| | 6 | 7.50 | 12.50 | 8.75 | 8.13 | 6.25 | 8.13 | 6.88 |
| | 7 | 15.03 | 14.38 | 14.38 | 14.38 | 13.73 | 10.46 | 8.50 |
| | 8 | 12.26 | 12.26 | 9.68 | 7.10 | 7.10 | 7.74 | 9.03 |
| 3 (CIA + | 1 | 9.93 | 3.97 | 5.96 | 3.97 | 7.28 | 7.95 | 8.61 |
| Dexamethasone) | 2 | −3.09 | −5.56 | −4.32 | −6.17 | −9.26 | −3.70 | −3.70 |
| | 3 | 2.30 | 7.47 | 8.05 | 8.62 | 10.34 | 5.75 | 7.47 |
| | 4 | 0.59 | −0.59 | −2.37 | −2.37 | 0.59 | 2.37 | 0.00 |
| | 5 | −1.20 | −4.22 | 0.60 | 0.00 | 1.20 | 0.60 | −3.01 |
| | 6 | −6.83 | −8.70 | −6.83 | −5.59 | −5.59 | −4.35 | −5.59 |
| | 7 | 2.33 | 1.16 | −1.16 | 4.07 | 5.81 | 4.65 | 2.91 |
| | 8 | −0.63 | 1.88 | 5.00 | 5.63 | 1.88 | 0.00 | 3.75 |
| 4 (CIA + | 1 | 9.55 | 8.28 | 7.01 | 4.46 | 2.55 | 3.82 | 3.18 |
| GSK620) | 2 | 0.00 | 0.00 | 0.00 | −1.19 | −1.79 | −2.98 | −2.98 |
| | 3 | 11.52 | 12.73 | 10.91 | 10.30 | 5.45 | 1.82 | 3.03 |
| | 4 | 6.79 | 5.56 | 5.56 | 3.70 | 4.94 | 1.23 | 1.85 |
| | 5 | 14.12 | 14.12 | 12.94 | 8.24 | 7.65 | 9.41 | 7.06 |
| | 6 | 8.07 | 6.21 | 3.11 | 0.00 | −3.73 | 0.00 | −0.62 |
| | 7 | 3.47 | 0.00 | −0.58 | −2.31 | −4.62 | −1.73 | −4.62 |
| | 8 | 8.43 | 3.61 | 1.81 | 2.41 | 2.41 | 1.81 | −0.60 |
| 5 (CIA + Example | 1 | 1.86 | 4.97 | 3.73 | 3.11 | 3.11 | 1.86 | 3.11 |
| 101 3 mg/kg) | 2 | −1.89 | −5.66 | −4.40 | −4.40 | −5.66 | −4.40 | −3.77 |
| | 3 | 5.85 | 0.58 | 1.17 | 2.34 | 1.17 | 1.17 | 2.92 |
| | 4 | 4.52 | 2.26 | 2.82 | 2.82 | 1.13 | 1.13 | 2.26 |
| | 5 | 9.03 | 9.68 | 9.68 | 7.74 | 3.87 | 3.23 | 4.52 |
| | 6 | 4.82 | 3.61 | 1.81 | 0.00 | −3.01 | 0.60 | 0.60 |
| | 7 | 10.30 | 7.88 | 6.06 | 3.64 | 3.03 | 2.42 | 1.82 |
| | 8 | 6.67 | 7.88 | 6.06 | 3.64 | 3.64 | 3.03 | 2.42 |
| 6 (CIA + Example | 1 | 1.17 | 0.00 | 1.17 | 0.00 | −0.58 | −2.34 | −2.92 |
| 101 10 mg/kg) | 2 | −5.13 | −5.13 | −7.05 | −7.69 | −6.41 | −7.69 | −8.97 |
| | 3 | −2.94 | −4.71 | −4.71 | −5.88 | −5.88 | −4.12 | −5.29 |
| | 4 | −0.62 | −0.62 | 0.62 | −1.23 | −1.23 | −0.62 | −0.62 |
| | 5 | 4.29 | 1.23 | −1.23 | −2.45 | −2.45 | −0.61 | −3.07 |
| | 6 | 12.82 | 7.69 | 7.05 | 7.69 | 5.77 | 7.05 | 5.77 |
| | 7 | 14.38 | 16.88 | 20.00 | 17.50 | 13.75 | 13.13 | 13.13 |
| | 8 | 2.48 | 2.48 | 2.48 | 3.11 | 3.11 | 5.59 | 6.21 |
| 7 (CIA + Example | 1 | 7.74 | 8.39 | 9.03 | 11.61 | 12.90 | 12.26 | 11.61 |
| 101 30 mg/kg) | 2 | 0.00 | −1.74 | −3.49 | −0.58 | −2.33 | −4.07 | −5.23 |
| | 3 | 0.00 | −2.42 | −1.21 | −1.82 | −1.21 | −4.24 | −4.24 |
| | 4 | 3.66 | 4.88 | 2.44 | 4.27 | 5.49 | 6.10 | 6.10 |
| | 5 | −4.27 | −3.05 | −3.05 | −3.05 | −3.05 | −2.44 | −1.83 |
| | 6 | 4.82 | 3.01 | 2.41 | 1.81 | −1.20 | −3.61 | −3.01 |
| | 7 | 3.90 | 0.65 | 1.30 | −1.30 | −1.95 | −1.95 | −2.60 |
| | 8 | 3.21 | 3.85 | 3.85 | 1.92 | 1.28 | 1.92 | 3.21 |

TABLE 13C
Mean animal body weight (g) ± SD

| Group | Day 0 | Day 1 | Day 2 | Day 3 | Day 4 | Day 5 | Day 6 | Day 7 |
|---|---|---|---|---|---|---|---|---|
| 1 (No CIA + | 166 ± | 167 ± | 169 ± | 170 ± | 174 ± | 176 ± | 179 ± | 179 ± |
| Vehicle) | 6 | 5 | 6 | 6 | 7 | 6 | 7 | 6 |
| 2 (CIA + | 161 ± | 164 ± | 164 ± | 167 ± | 168 ± | 172 ± | 176 ± | 176 ± |
| Vehicle) | 8 | 9 | 9 | 10 | 8 | 10 | 8 | 9 |
| 3 (CIA + | 164 ± | 160 ± | 159 ± | 157 ± | 156 ± | 156 ± | 157 ± | 159 ± |
| Dexamethasone) | 7 | 8 | 10 | 11 | 9** | 8** | 8** | 10** |
| 4 (CIA + | 165 ± | 170 ± | 169 ± | 170 ± | 170 ± | 175 ± | 174 ± | 175 ± |
| GSK620) | 5 | 6 | 5 | 6 | 6 | 5 | 6 | 5 |
| 5 (CIA + Example | 165 ± | 164 ± | 165 ± | 167 ± | 166 ± | 168 ± | 171 ± | 173 ± |
| 101 3 mg/kg) | 7 | 9 | 9 | 7 | 8 | 8 | 9 | 7 |
| 6 (CIA + Example | 162 ± | 160 ± | 160 ± | 164 ± | 164 ± | 165 ± | 165 ± | 166 ± |
| 101 10 mg/kg) | 6 | 8 | 8 | 9 | 8 | 10 | 11** | 11** |
| 7(CIA + Example | 162 ± | 161 ± | 160 ± | 160 ± | 163 ± | 163 ± | 162 ± | 162 ± |
| 101 30 mg/kg) | 6 | 7 | 6 | 5 | 4 | 5* | 4** | 4** |

| Group | Day 8 | Day 9 | Day 10 | Day 11 | Day 12 | Day 13 | Day 14 |
|---|---|---|---|---|---|---|---|
| 1 (No CIA + | 180 ± | 184 ± | 185 ± | 187 ± | 189 ± | 190 ± | 191 ± |
| Vehicle) | 8 | 7 | 6 | 7 | 8 | 9 | 9 |
| 2 (CIA + | 176 ± | 180 ± | 181 ± | 182 ± | 184 ± | 187 ± | 185 ± |
| Vehicle) | 9 | 10 | 8 | 7 | 10 | 12 | 12 |
| 3 (CIA + | 158 ± | 162 ± | 161 ± | 162 ± | 163 ± | 163 ± | 162 ± |
| Dexamethasone) | 10 | 10** | 10** | 12** | 12** | 13** | 12** |
| 4 (CIA + | 177 ± | 179 ± | 180 ± | 181 ± | 183 ± | 183 ± | 180 ± |
| GSK620) | 5 | 5 | 7 | 5 | 4 | 6 | 6 |
| 5 (CIA + Example | 173 ± | 174 ± | 176 ± | 178 ± | 178 ± | 178 ± | 177 ± |
| 101 3 mg/kg) | 8 | 8 | 8 | 9 | 9 | 9 | 9 |
| 6 (CIA + Example | 167 ± | 170 ± | 170 ± | 171 ± | 169 ± | 169 ± | 169 ± |
| 101 10 mg/kg) | 12 | 10* | 10** | 12** | 14** | 12** | 12** |
| 7 (CIA + Example | 164 ± | 166 ± | 166 ± | 165 ± | 163 ± | 162 ± | 164 ± |
| 101 30 mg/kg) | 4 | 4** | 5** | 5** | 6** | 6** | 5** |
| Group | Day 15 | Day 16 | Day 17 | Day 18 | Day 19 | Day 20 | Day 21 |
| 1 (No CIA + | 192 ± | 193 ± | 194 ± | 194 ± | 196 ± | 198 ± | 200 ± |
| Vehicle) | 8* | 9* | 11** | 10** | 10** | 11** | 12** |
| 2 (CIA + | 181 ± | 182 ± | 180 ± | 178 ± | 176 ± | 174 ± | 173 ± |
| Vehicle) | 11 | 11 | 11 | 10 | 10 | 9 | 10 |
| 3 (CIA + | 165 ± | 164 ± | 165 ± | 166 ± | 167 ± | 167 ± | 167 ± |
| Dexamethasone) | 10** | 13** | 11** | 13* | 15 | 11 | 11 |
| 4 (CIA + | 178 ± | 176 ± | 174 ± | 171 ± | 168 ± | 168 ± | 167 ± |
| GSK620) | 8 | 9 | 9 | 8 | 8 | 8 | 7 |
| 5 (CIA + Example | 173 ± | 171 ± | 170 ± | 169 ± | 166 ± | 167 ± | 168 ± |
| 101 3 mg/kg) | 10 | 10* | 9* | 9 | 9 | 8 | 8 |
| 6 (CIA + Example | 168 ± | 166 ± | 166 ± | 165 ± | 164 ± | 164 ± | 163 ± |
| 101 10 mg/kg) | 11** | 11** | 13** | 13** | 10* | 10* | 11* |
| 7 (CIA + Example | 166 ± | 165 ± | 164 ± | 165 ± | 164 ± | 163 ± | 163 ± |
| 101 30 mg/kg) | 6** | 6** | 5** | 8** | 8* | 8* | 8* |

One-way ANOVA comparison to Group 2;
*p < 0.05;
**p < 0.01

TABLE 13D
Average animal body weight percent change (%)

| Group | Day 0 | Day 1 | Day 2 | Day 3 | Day 4 | Day 5 | Day 6 | Day 7 |
|---|---|---|---|---|---|---|---|---|
| 1 (No CIA + | 0.00 | 0.85 | 1.75 | 2.50 | 4.53 | 6.13 | 7.83 | 8.08 |
| Vehicle) | | | | | | | | |
| 2 (CIA + | 0.00 | 1.78 | 1.70 | 3.55 | 4.37 | 6.56 | 9.44 | 9.66 |
| Vehicle) | | | | | | | | |
| 3 (CIA + | 0.00 | −2.80 | −3.22 | −4.37 | −4.95 | −4.95 | −4.41 | −3.35 |
| Dexamethasone) | | | | | | | | |
| 4 (CIA + | 0.00 | 2.94 | 2.43 | 3.12 | 3.03 | 6.00 | 5.46 | 5.69 |
| GSK620) | | | | | | | | |
| 5 (CIA + Example | 0.00 | −0.49 | −0.15 | 1.01 | 0.94 | 1.81 | 3.40 | 4.80 |
| 101 3 mg/kg) | | | | | | | | |
| 6 (CIA + Example | 0.00 | −1.45 | −1.29 | 0.91 | 0.99 | 1.43 | 1.77 | 2.23 |
| 101 10 mg/kg) | | | | | | | | |
| 7 (CIA + Example | 0.00 | −0.83 | −1.03 | −1.32 | 0.47 | 0.90 | 0.03 | 0.29 |
| 101 30 mg/kg) | | | | | | | | |

| Group | Day 8 | Day 9 | Day 10 | Day 11 | Day 12 | Day 13 | Day 14 |
|---|---|---|---|---|---|---|---|
| 1 (No CIA + | 8.21 | 10.64 | 11.40 | 12.43 | 13.88 | 14.68 | 14.91 |
| Vehicle) | | | | | | | |
| 2 (CIA + | 9.34 | 11.63 | 12.22 | 13.27 | 14.60 | 15.98 | 14.89 |
| Vehicle) | | | | | | | |
| 3 (CIA + | −3.90 | −1.84 | −2.30 | −1.24 | −1.18 | −0.91 | −1.24 |
| Dexamethasone) | | | | | | | |
| 4 (CIA + | 6.89 | 8.34 | 8.62 | 9.70 | 10.85 | 10.45 | 8.63 |
| GSK620) | | | | | | | |
| 5 (CIA + Example | 4.69 | 5.59 | 6.84 | 7.79 | 8.18 | 7.74 | 7.08 |
| 101 3 mg/kg) | | | | | | | |
| 6 (CIA + Example | 2.82 | 4.86 | 4.86 | 5.09 | 4.18 | 3.92 | 4.05 |
| 101 10 mg/kg) | | | | | | | |
| 7 (CIA + Example | 1.21 | 2.81 | 2.27 | 2.22 | 0.66 | 0.18 | 1.42 |
| 101 30 mg/kg) | | | | | | | |
| Group | Day 15 | Day 16 | Day 17 | Day 18 | Day 19 | Day 20 | Day 21 |
| 1 (No CIA + | 15.52 | 16.10 | 16.97 | 17.09 | 18.20 | 19.32 | 20.14 |
| Vehicle) | | | | | | | |
| 2 (CIA + | 12.71 | 13.10 | 12.00 | 10.64 | 9.17 | 8.01 | 7.37 |
| Vehicle) | | | | | | | |
| 3 (CIA + | 0.43 | −0.57 | 0.62 | 1.02 | 1.53 | 1.66 | 1.30 |
| Dexamethasone) | | | | | | | |
| 4 (CIA + | 7.74 | 6.31 | 5.09 | 3.20 | 1.61 | 1.67 | 0.79 |
| GSK620) | | | | | | | |
| 5 (CIA + Example | 5.15 | 3.90 | 3.37 | 2.36 | 0.91 | 1.13 | 1.73 |
| 101 3 mg/kg) | | | | | | | |
| 6 (CIA + Example | 3.31 | 2.23 | 2.29 | 1.38 | 0.76 | 1.30 | 0.53 |
| 101 10 mg/kg) | | | | | | | |
| 7 (CIA + Example | 2.38 | 1.69 | 1.41 | 1.61 | 1.24 | 0.50 | 0.50 |
| 101 30 mg/kg) | | | | | | | |

TABLE 14A
Animal paw volume (mL)

| | Animal | Day 0 | | Day 6 | | Day 12 | | Day 13 | | Day 14 | |
|---|---|---|---|---|---|---|---|---|---|---|---|
| Group | # | L | R | L | R | L | R | L | R | L | R |
| 1 (No CIA + | 1 | 1.32 | 1.34 | 1.36 | 1.37 | 1.38 | 1.38 | 1.38 | 1.41 | 1.37 | 1.41 |
| Vehicle) | 2 | 1.31 | 1.32 | 1.32 | 1.32 | 1.37 | 1.39 | 1.37 | 1.40 | 1.38 | 1.42 |
| | 3 | 1.31 | 1.31 | 1.33 | 1.34 | 1.37 | 1.38 | 1.38 | 1.40 | 1.39 | 1.42 |
| | 4 | 1.28 | 1.29 | 1.32 | 1.31 | 1.41 | 1.42 | 1.43 | 1.45 | 1.43 | 1.46 |
| | 5 | 1.36 | 1.34 | 1.36 | 1.34 | 1.46 | 1.49 | 1.48 | 1.49 | 1.51 | 1.49 |
| | 6 | 1.28 | 1.29 | 1.30 | 1.30 | 1.36 | 1.35 | 1.37 | 1.36 | 1.38 | 1.38 |
| | 7 | 1.33 | 1.32 | 1.33 | 1.32 | 1.37 | 1.39 | 1.39 | 1.40 | 1.40 | 1.42 |
| | 8 | 1.31 | 1.32 | 1.34 | 1.33 | 1.38 | 1.37 | 1.38 | 1.39 | 1.40 | 1.40 |
| 2 (CIA + | 1 | 1.35 | 1.35 | 1.38 | 1.38 | 1.86 | 1.83 | 2.07 | 1.94 | 2.55 | 2.42 |
| Vehicle) | 2 | 1.33 | 1.33 | 1.36 | 1.36 | 1.47 | 1.45 | 1.49 | 1.48 | 1.69 | 1.99 |
| | 3 | 1.30 | 1.31 | 1.32 | 1.33 | 1.45 | 1.46 | 1.49 | 1.49 | 1.49 | 1.49 |
| | 4 | 1.35 | 1.35 | 1.38 | 1.38 | 2.13 | 1.90 | 2.42 | 2.03 | 2.66 | 2.60 |
| | 5 | 1.33 | 1.32 | 1.36 | 1.34 | 1.47 | 1.48 | 1.41 | 1.49 | 1.99 | 1.69 |
| | 6 | 1.31 | 1.30 | 1.34 | 1.33 | 1.45 | 1.44 | 1.45 | 1.46 | 1.49 | 1.50 |
| | 7 | 1.29 | 1.30 | 1.30 | 1.32 | 1.48 | 1.48 | 1.82 | 1.53 | 2.21 | 1.83 |
| | 8 | 1.30 | 1.30 | 1.31 | 1.30 | 1.46 | 1.41 | 1.74 | 1.47 | 2.14 | 1.49 |
| 3 (CIA + | 1 | 1.35 | 1.34 | 1.31 | 1.30 | 1.21 | 1.25 | 1.23 | 1.26 | 1.24 | 1.26 |
| Dexamethasone) | 2 | 1.31 | 1.30 | 1.29 | 1.28 | 1.21 | 1.26 | 1.24 | 1.27 | 1.24 | 1.26 |
| | 3 | 1.39 | 1.38 | 1.34 | 1.34 | 1.37 | 1.37 | 1.40 | 1.38 | 1.39 | 1.38 |
| | 4 | 1.33 | 1.35 | 1.29 | 1.30 | 1.28 | 1.25 | 1.29 | 1.25 | 1.27 | 1.23 |
| | 5 | 1.30 | 1.29 | 1.26 | 1.27 | 1.28 | 1.27 | 1.30 | 1.28 | 1.28 | 1.26 |
| | 6 | 1.30 | 1.30 | 1.26 | 1.26 | 1.22 | 1.18 | 1.25 | 1.21 | 1.24 | 1.20 |
| | 7 | 1.34 | 1.36 | 1.28 | 1.28 | 1.30 | 1.26 | 1.31 | 1.31 | 1.29 | 1.29 |
| | 8 | 1.36 | 1.37 | 1.29 | 1.30 | 1.29 | 1.29 | 1.29 | 1.29 | 1.25 | 1.26 |
| 4 (CIA + | 1 | 1.27 | 1.27 | 1.27 | 1.27 | 1.33 | 1.31 | 1.34 | 1.40 | 1.55 | 1.74 |
| GSK620) | 2 | 1.32 | 1.31 | 1.34 | 1.35 | 1.33 | 1.35 | 1.34 | 1.35 | 1.78 | 1.49 |
| | 3 | 1.27 | 1.28 | 1.37 | 1.38 | 1.41 | 1.42 | 1.43 | 1.42 | 1.44 | 1.44 |
| | 4 | 1.37 | 1.37 | 1.36 | 1.37 | 1.37 | 1.39 | 1.36 | 1.57 | 1.36 | 2.11 |
| | 5 | 1.37 | 1.37 | 1.38 | 1.39 | 1.42 | 1.39 | 1.43 | 1.42 | 1.43 | 1.43 |
| | 6 | 1.34 | 1.33 | 1.34 | 1.33 | 1.36 | 1.38 | 1.37 | 1.37 | 1.40 | 1.38 |
| | 7 | 1.32 | 1.33 | 1.32 | 1.33 | 1.35 | 1.38 | 1.38 | 1.41 | 1.73 | 1.99 |
| | 8 | 1.35 | 1.34 | 1.35 | 1.34 | 1.38 | 1.37 | 1.40 | 1.39 | 1.73 | 1.83 |
| 5 (CIA + Example | 1 | 1.36 | 1.38 | 1.36 | 1.38 | 1.38 | 1.40 | 1.62 | 1.42 | 1.67 | 1.57 |
| 101 3 mg/kg) | 2 | 1.31 | 1.32 | 1.32 | 1.33 | 1.32 | 1.43 | 1.53 | 1.50 | 1.68 | 1.52 |
| | 3 | 1.32 | 1.34 | 1.32 | 1.35 | 1.39 | 1.38 | 1.50 | 1.41 | 1.68 | 1.41 |
| | 4 | 1.32 | 1.32 | 1.40 | 1.40 | 1.42 | 1.46 | 1.54 | 1.47 | 1.64 | 1.87 |
| | 5 | 1.35 | 1.34 | 1.36 | 1.36 | 1.37 | 1.38 | 1.38 | 1.40 | 1.59 | 1.50 |
| | 6 | 1.31 | 1.31 | 1.31 | 1.32 | 1.63 | 1.44 | 1.86 | 1.68 | 2.01 | 2.04 |
| | 7 | 1.37 | 1.35 | 1.37 | 1.36 | 1.39 | 1.36 | 1.43 | 1.42 | 1.63 | 1.43 |
| | 8 | 1.34 | 1.34 | 1.35 | 1.34 | 1.39 | 1.39 | 1.41 | 1.39 | 1.63 | 1.43 |
| 6 (CIA + Example | 1 | 1.39 | 1.41 | 1.41 | 1.41 | 1.52 | 1.44 | 1.71 | 1.54 | 2.00 | 1.62 |
| 101 10 mg/kg) | 2 | 1.26 | 1.26 | 1.23 | 1.24 | 1.26 | 1.27 | 1.32 | 1.32 | 1.50 | 1.68 |
| | 3 | 1.29 | 1.30 | 1.31 | 1.32 | 1.43 | 1.88 | 1.81 | 1.90 | 2.07 | 2.23 |
| | 4 | 1.29 | 1.30 | 1.33 | 1.33 | 1.63 | 1.33 | 1.83 | 1.37 | 2.45 | 1.67 |
| | 5 | 1.31 | 1.30 | 1.34 | 1.34 | 1.34 | 1.35 | 1.35 | 1.37 | 1.39 | 1.37 |
| | 6 | 1.30 | 1.31 | 1.35 | 1.34 | 1.44 | 1.38 | 1.45 | 1.41 | 1.45 | 1.43 |
| | 7 | 1.37 | 1.37 | 1.37 | 1.36 | 1.39 | 1.39 | 1.44 | 1.44 | 1.44 | 1.46 |
| | 8 | 1.26 | 1.26 | 1.26 | 1.26 | 1.27 | 1.29 | 1.31 | 1.29 | 1.31 | 1.28 |
| 7 (CIA + Example | 1 | 1.33 | 1.34 | 1.35 | 1.36 | 1.36 | 1.50 | 1.40 | 1.54 | 1.40 | 1.53 |
| 101 30 mg/kg) | 2 | 1.32 | 1.31 | 1.33 | 1.32 | 1.34 | 1.36 | 1.36 | 1.38 | 1.35 | 1.37 |
| | 3 | 1.33 | 1.31 | 1.33 | 1.31 | 1.31 | 1.33 | 1.33 | 1.34 | 1.32 | 1.35 |
| | 4 | 1.32 | 1.33 | 1.33 | 1.34 | 1.33 | 1.34 | 1.34 | 1.36 | 1.37 | 1.36 |
| | 5 | 1.32 | 1.30 | 1.32 | 1.32 | 1.36 | 1.33 | 1.38 | 1.36 | 1.37 | 1.36 |
| | 6 | 1.31 | 1.32 | 1.32 | 1.33 | 1.32 | 1.33 | 1.34 | 1.34 | 1.35 | 1.36 |
| | 7 | 1.30 | 1.32 | 1.32 | 1.33 | 1.34 | 1.35 | 1.36 | 1.36 | 1.36 | 1.35 |
| | 8 | 1.34 | 1.34 | 1.34 | 1.34 | 1.35 | 1.33 | 1.35 | 1.33 | 1.30 | 1.28 |

| | Animal | Day 15 | | Day 18 | | Day 19 | | Day 21 | |
|---|---|---|---|---|---|---|---|---|---|
| Group | # | L | R | L | R | L | R | L | R |
| 1 (No CIA + | 1 | 1.38 | 1.42 | 1.39 | 1.40 | 1.38 | 1.40 | 1.38 | 1.38 |
| Vehicle) | 2 | 1.38 | 1.42 | 1.39 | 1.40 | 1.38 | 1.40 | 1.38 | 1.38 |
| | 3 | 1.42 | 1.42 | 1.42 | 1.42 | 1.41 | 1.42 | 1.42 | 1.42 |
| | 4 | 1.43 | 1.45 | 1.42 | 1.44 | 1.41 | 1.41 | 1.42 | 1.43 |
| | 5 | 1.53 | 1.51 | 1.52 | 1.51 | 1.51 | 1.50 | 1.54 | 1.52 |
| | 6 | 1.39 | 1.38 | 1.39 | 1.38 | 1.39 | 1.40 | 1.40 | 1.40 |
| | 7 | 1.40 | 1.43 | 1.41 | 1.42 | 1.41 | 1.41 | 1.40 | 1.40 |
| | 8 | 1.40 | 1.42 | 1.42 | 1.41 | 1.41 | 1.40 | 1.41 | 1.40 |
| 2 (CIA + | 1 | 2.70 | 2.54 | 2.71 | 2.72 | 2.74 | 2.73 | 2.72 | 2.71 |
| Vehicle) | 2 | 2.14 | 2.22 | 2.77 | 3.00 | 2.92 | 3.02 | 3.03 | 3.03 |
| | 3 | 1.69 | 2.05 | 2.87 | 2.88 | 3.03 | 3.25 | 3.04 | 3.22 |
| | 4 | 3.09 | 2.89 | 3.21 | 3.18 | 3.26 | 3.23 | 3.26 | 3.22 |
| | 5 | 2.29 | 2.16 | 2.94 | 2.75 | 2.94 | 2.74 | 2.94 | 2.75 |
| | 6 | 1.92 | 1.69 | 2.37 | 2.53 | 2.54 | 2.52 | 2.64 | 2.54 |
| | 7 | 2.67 | 2.05 | 2.69 | 2.68 | 2.66 | 2.63 | 2.66 | 2.64 |
| | 8 | 2.62 | 1.65 | 2.62 | 2.30 | 2.62 | 2.63 | 2.60 | 2.64 |
| 3 (CIA + | 1 | 1.23 | 1.25 | 1.28 | 1.28 | 1.28 | 1.27 | 1.26 | 1.26 |
| Dexamethasone) | 2 | 1.21 | 1.24 | 1.22 | 1.24 | 1.22 | 1.22 | 1.22 | 1.22 |
| | 3 | 1.36 | 1.36 | 1.45 | 1.44 | 1.44 | 1.44 | 1.43 | 1.43 |
| | 4 | 1.28 | 1.27 | 1.31 | 1.31 | 1.30 | 1.31 | 1.31 | 1.31 |
| | 5 | 1.26 | 1.27 | 1.29 | 1.29 | 1.28 | 1.27 | 1.30 | 1.30 |
| | 6 | 1.16 | 1.14 | 1.18 | 1.17 | 1.19 | 1.18 | 1.18 | 1.18 |
| | 7 | 1.33 | 1.32 | 1.34 | 1.34 | 1.33 | 1.32 | 1.31 | 1.32 |
| | 8 | 1.27 | 1.26 | 1.30 | 1.29 | 1.30 | 1.30 | 1.30 | 1.30 |
| 4 (CIA + | 1 | 2.04 | 2.05 | 2.72 | 2.62 | 2.71 | 2.59 | 2.71 | 2.63 |
| GSK620) | 2 | 2.03 | 2.08 | 2.54 | 2.45 | 2.54 | 2.46 | 2.51 | 2.44 |
| | 3 | 1.39 | 1.38 | 2.39 | 2.41 | 2.32 | 2.33 | 2.34 | 2.21 |
| | 4 | 1.49 | 2.42 | 2.87 | 3.15 | 3.06 | 3.15 | 3.04 | 3.08 |
| | 5 | 1.52 | 1.47 | 2.29 | 1.46 | 2.61 | 1.46 | 2.62 | 1.61 |
| | 6 | 1.60 | 1.56 | 2.05 | 2.04 | 2.19 | 2.20 | 2.21 | 2.18 |
| | 7 | 2.14 | 2.13 | 2.66 | 2.38 | 2.63 | 2.36 | 2.54 | 2.37 |
| | 8 | 1.75 | 1.79 | 2.34 | 2.34 | 2.32 | 2.33 | 2.32 | 2.33 |
| 5 (CIA + Example | 1 | 1.68 | 1.64 | 2.22 | 2.71 | 2.79 | 2.76 | 2.87 | 2.77 |
| 101 3 mg/kg) | 2 | 1.72 | 1.66 | 2.40 | 2.21 | 2.51 | 2.21 | 2.49 | 2.23 |
| | 3 | 1.70 | 1.62 | 2.65 | 2.56 | 2.85 | 2.91 | 3.01 | 2.91 |
| | 4 | 1.91 | 2.31 | 2.62 | 2.65 | 2.75 | 2.63 | 2.76 | 2.61 |
| | 5 | 1.65 | 1.71 | 2.44 | 2.37 | 2.50 | 2.37 | 2.51 | 2.34 |
| | 6 | 2.44 | 2.41 | 2.70 | 2.70 | 2.71 | 2.68 | 2.68 | 2.69 |
| | 7 | 2.06 | 2.02 | 2.47 | 2.55 | 2.60 | 2.80 | 2.64 | 2.77 |
| | 8 | 1.74 | 1.99 | 2.44 | 2.50 | 2.43 | 2.49 | 2.41 | 2.47 |
| 6 (CIA + Example | 1 | 2.13 | 1.93 | 2.40 | 2.23 | 2.38 | 2.36 | 2.38 | 2.35 |
| 101 10 mg/kg) | 2 | 1.73 | 1.83 | 2.04 | 2.08 | 2.04 | 2.05 | 2.03 | 2.04 |
| | 3 | 2.33 | 2.53 | 2.54 | 2.52 | 2.49 | 2.47 | 2.48 | 2.46 |
| | 4 | 2.49 | 1.91 | 2.70 | 2.74 | 2.64 | 2.71 | 2.64 | 2.68 |
| | 5 | 1.41 | 1.38 | 1.51 | 1.59 | 1.48 | 1.58 | 1.48 | 1.58 |
| | 6 | 1.60 | 1.45 | 2.30 | 1.91 | 2.32 | 1.87 | 2.33 | 2.15 |
| | 7 | 1.49 | 1.51 | 1.64 | 1.58 | 1.65 | 1.57 | 1.83 | 1.82 |
| | 8 | 1.51 | 1.52 | 1.51 | 1.51 | 1.60 | 1.56 | 1.60 | 1.62 |
| 7 (CIA + Example | 1 | 1.46 | 1.52 | 1.46 | 1.51 | 1.42 | 1.44 | 1.43 | 1.43 |
| 101 30 mg/kg) | 2 | 1.79 | 1.37 | 2.05 | 1.87 | 2.04 | 1.96 | 2.08 | 2.10 |
| | 3 | 1.57 | 1.68 | 1.90 | 1.86 | 2.06 | 2.14 | 2.11 | 2.16 |
| | 4 | 1.38 | 1.38 | 1.47 | 1.37 | 1.56 | 1.39 | 1.54 | 1.40 |
| | 5 | 1.38 | 1.49 | 1.47 | 1.48 | 1.55 | 1.48 | 1.54 | 1.47 |
| | 6 | 1.34 | 1.36 | 1.68 | 1.32 | 1.85 | 1.83 | 2.06 | 2.00 |
| | 7 | 1.44 | 1.73 | 1.93 | 1.95 | 1.94 | 2.04 | 2.13 | 2.12 |
| | 8 | 1.31 | 1.30 | 1.70 | 1.68 | 1.67 | 1.69 | 1.67 | 1.67 |

TABLE 14B
Animal paw volume percent change (%)

| | | | | | |
|---|---|---|---|---|---|
| Animal | Day 0 | Day 6 | Day 12 | Day 13 | Day 14 |

| Group | # | L | R | L | R | L | R | L | R | L | R |
|---|---|---|---|---|---|---|---|---|---|---|---|
| 1 (No CIA + | 1 | 0.00 | 0.00 | 3.04 | 2.25 | 4.56 | 3.37 | 4.56 | 5.24 | 3.80 | 5.24 |
| Vehicle) | 2 | 0.00 | 0.00 | 0.77 | 0.38 | 4.60 | 5.32 | 4.60 | 6.46 | 5.75 | 7.60 |
| | 3 | 0.00 | 0.00 | 1.53 | 2.30 | 4.60 | 5.36 | 5.36 | 6.90 | 6.51 | 8.43 |
| | 4 | 0.00 | 0.00 | 3.14 | 1.56 | 10.20 | 10.51 | 11.76 | 12.45 | 12.16 | 13.62 |
| | 5 | 0.00 | 0.00 | 0.00 | 0.37 | 7.75 | 11.24 | 9.23 | 11.24 | 11.44 | 11.24 |
| | 6 | 0.00 | 0.00 | 1.57 | 1.17 | 6.67 | 4.67 | 7.06 | 5.45 | 8.24 | 7.00 |
| | 7 | 0.00 | 0.00 | 0.00 | 0.00 | 3.02 | 5.32 | 4.53 | 6.08 | 5.28 | 7.98 |
| | 8 | 0.00 | 0.00 | 1.91 | 1.14 | 4.96 | 4.18 | 4.96 | 5.32 | 6.49 | 6.08 |
| 2 (CIA + | 1 | 0.00 | 0.00 | 2.23 | 2.60 | 37.92 | 35.69 | 53.90 | 43.87 | 89.22 | 79.55 |
| Vehicle) | 2 | 0.00 | 0.00 | 1.88 | 2.64 | 10.15 | 9.43 | 11.65 | 11.32 | 26.69 | 50.19 |
| | 3 | 0.00 | 0.00 | 1.54 | 1.15 | 11.58 | 11.07 | 14.67 | 13.36 | 14.67 | 13.36 |
| | 4 | 0.00 | 0.00 | 2.23 | 2.23 | 57.99 | 41.26 | 79.55 | 50.56 | 97.40 | 92.94 |
| | 5 | 0.00 | 0.00 | 1.88 | 1.52 | 10.15 | 11.74 | 6.02 | 12.50 | 49.25 | 27.65 |
| | 6 | 0.00 | 0.00 | 1.91 | 2.31 | 10.31 | 10.38 | 10.31 | 11.92 | 13.36 | 15.00 |
| | 7 | 0.00 | 0.00 | 1.17 | 1.15 | 15.18 | 13.85 | 41.25 | 17.31 | 71.60 | 40.38 |
| | 8 | 0.00 | 0.00 | 0.77 | 0.00 | 11.92 | 8.08 | 33.46 | 12.69 | 64.23 | 14.23 |
| 3 (CIA + | 1 | 0.00 | 0.00 | −2.97 | −3.00 | −10.41 | −6.74 | −8.92 | −5.99 | −8.18 | −5.62 |
| Dexamethasone) | 2 | 0.00 | 0.00 | −1.53 | −1.16 | −7.66 | −3.09 | −5.36 | −2.32 | −5.36 | −2.70 |
| | 3 | 0.00 | 0.00 | −3.61 | 2.91 | −1.44 | −0.73 | 0.72 | 0.36 | 0.00 | 0.00 |
| | 4 | 0.00 | 0.00 | −3.02 | I3.72 | −3.40 | −7.43 | −3.02 | −7.43 | −4.53 | −8.92 |
| | 5 | 0.00 | 0.00 | −3.09 | −1.56 | −1.54 | −1.56 | 0.00 | −0.78 | −1.54 | −2.33 |
| | 6 | 0.00 | 0.00 | −3.08 | 12.70 | −6.54 | −9.27 | −4.23 | −6.95 | −5.00 | −7.72 |
| | 7 | 0.00 | 0.00 | −4.12 | −5.90 | −3.00 | −7.01 | −2.25 | −3.69 | −3.75 | −5.17 |
| | 8 | 0.00 | 0.00 | −5.51 | −5.13 | −5.51 | −5.86 | −5.51 | −5.86 | −8.46 | −8.06 |
| 4 (CIA + | 1 | 0.00 | 0.00 | −0.39 | −0.39 | 4.33 | 2.76 | 5.12 | 9.84 | 21.65 | 37.01 |
| GSK620) | 2 | 0.00 | 0.00 | 1.52 | 3.07 | 0.76 | 3.07 | 1.52 | 3.07 | 34.98 | 14.18 |
| | 3 | 0.00 | 0.00 | 7.91 | 7.84 | 11.07 | 10.98 | 12.65 | 10.98 | 13.44 | 12.55 |
| | 4 | 0.00 | 0.00 | −0.73 | 0.00 | 0.00 | 1.47 | −0.73 | 14.65 | −0.37 | 54.58 |
| | 5 | 0.00 | 0.00 | 0.36 | 1.47 | 3.28 | 1.47 | 4.01 | 3.66 | 4.01 | 4.40 |
| | 6 | 0.00 | 0.00 | 0.00 | 0.00 | 1.50 | 3.77 | 2.25 | 3.02 | 4.87 | 3.77 |
| | 7 | 0.00 | 0.00 | 0.00 | 0.00 | 2.28 | 3.77 | 4.56 | 6.04 | 31.18 | 49.81 |
| | 8 | 0.00 | 0.00 | −0.37 | 0.00 | 1.85 | 2.25 | 3.33 | 3.75 | 27.78 | 36.70 |
| 5 (CIA + Example | 1 | 0.00 | 0.00 | 0.00 | 0.00 | 1.48 | 1.45 | 19.19 | 2.91 | 22.88 | 13.82 |
| 101 3 mg/kg) | 2 | 0.00 | 0.00 | 0.77 | 0.76 | 0.77 | 8.37 | 16.86 | 13.69 | 28.74 | 15.21 |
| | 3 | 0.00 | 0.00 | 0.38 | 0.75 | 5.32 | 3.00 | 14.07 | 5.24 | 27.76 | 5.62 |
| | 4 | 0.00 | 0.00 | 6.08 | 5.68 | 7.60 | 10.23 | 16.73 | 10.98 | 24.71 | 41.29 |
| | 5 | 0.00 | 0.00 | 0.74 | 1.12 | 1.49 | 2.61 | 2.23 | 4.10 | 17.84 | 11.57 |
| | 6 | 0.00 | 0.00 | 0.00 | 0.77 | 24.52 | 9.96 | 42.15 | 28.35 | 53.64 | 55.94 |
| | 7 | 0.00 | 0.00 | 0.00 | 0.74 | 1.47 | 0.74 | 4.40 | 5.20 | 19.05 | 5.95 |
| | 8 | 0.00 | 0.00 | 0.75 | 0.00 | 3.75 | 3.75 | 5.24 | 3.75 | 21.72 | 6.74 |
| 6 (CIA + Example | 1 | 0.00 | 0.00 | 1.44 | 0.00 | 9.39 | 2.49 | 23.10 | 9.25 | 44.40 | 14.95 |
| 101 10 mg/kg) | 2 | 0.00 | 0.00 | −2.78 | −1.59 | −0.40 | 1.20 | 4.37 | 5.18 | 19.05 | 33.47 |
| | 3 | 0.00 | 0.00 | 1.56 | 1.54 | 10.89 | 44.79 | 40.47 | 46.33 | 60.70 | 71.81 |
| | 4 | 0.00 | 0.00 | 3.50 | 2.32 | 26.46 | 2.32 | 42.02 | 5.41 | 90.66 | 28.57 |
| | 5 | 0.00 | 0.00 | 2.30 | 3.09 | 2.30 | 3.86 | 3.07 | 5.41 | 6.13 | 5.41 |
| | 6 | 0.00 | 0.00 | 3.46 | 2.30 | 10.38 | 5.36 | 11.15 | 8.05 | 11.15 | 9.20 |
| | 7 | 0.00 | 0.00 | 0.00 | −0.73 | 1.47 | 1.47 | 5.13 | 5.13 | 5.49 | 6.59 |
| | 8 | 0.00 | 0.00 | −0.40 | 0.00 | 0.40 | 2.39 | 3.57 | 2.39 | 3.57 | 1.59 |
| 7 (CIA + Example | 1 | 0.00 | 0.00 | 1.89 | 1.50 | 2.26 | 12.36 | 5.28 | 14.98 | 5.28 | 14.61 |
| 101 30 mg/kg) | 2 | 0.00 | 0.00 | 0.76 | 0.77 | 1.52 | 3.83 | 3.04 | 5.36 | 2.28 | 4.60 |
| | 3 | 0.00 | 0.00 | 0.00 | 0.38 | −1.13 | 1.53 | 0.00 | 2.30 | −0.75 | 3.07 |
| | 4 | 0.00 | 0.00 | 0.76 | 0.64 | 0.76 | 0.64 | 1.52 | 2.15 | 4.18 | 2.15 |
| | 5 | 0.00 | 0.00 | 0.38 | 1.15 | 3.04 | 1.92 | 4.56 | 4.62 | 3.80 | 4.62 |
| | 6 | 0.00 | 0.00 | 0.38 | 0.76 | 0.38 | 0.76 | 2.29 | 1.52 | 2.67 | 3.04 |
| | 7 | 0.00 | 0.00 | 1.54 | 0.76 | 3.09 | 2.66 | 4.63 | 3.04 | 4.63 | 2.66 |
| | 8 | 0.00 | 0.00 | 0.37 | 0.00 | 0.75 | −0.75 | 0.75 | −0.75 | −3.00 | −4.12 |

| | Animal | Day 15 | | Day 18 | | Day 19 | | Day 21 | |
|---|---|---|---|---|---|---|---|---|---|
| Group | # | L | R | L | R | L | R | L | R |
| 1 (No CIA + | 1 | 4.56 | 5.99 | 5.32 | 4.49 | 4.56 | 4.49 | 4.56 | 3.00 |
| Vehicle) | 2 | 5.36 | 7.60 | 6.13 | 6.08 | 5.36 | 6.08 | 5.36 | 4.56 |
| | 3 | 8.43 | 8.43 | 8.43 | 8.43 | 7.66 | 8.43 | 8.43 | 8.43 |
| | 4 | 11.76 | 12.45 | 10.98 | 11.67 | 10.59 | 9.73 | 10.98 | 10.89 |
| | 5 | 12.55 | 12.73 | 11.81 | 12.73 | 11.07 | 12.36 | 13.28 | 13.48 |
| | 6 | 8.63 | 7.00 | 8.63 | 7.00 | 9.02 | 8.56 | 9.41 | 8.56 |
| | 7 | 5.28 | 8.37 | 6.04 | 7.60 | 6.04 | 6.84 | 5.28 | 6.08 |
| | 8 | 6.49 | 7.60 | 8.02 | 6.84 | 7.25 | 6.08 | 7.25 | 6.08 |
| 2 (CIA + | 1 | 100.37 | 88.48 | 101.12 | 101.86 | 103.35 | 102.60 | 102.23 | 101.12 |
| Vehicle) | 2 | 60.90 | 67.17 | 107.89 | 126.04 | 119.55 | 127.92 | 127.44 | 128.30 |
| | 3 | 30.12 | 56.11 | 121.24 | 119.85 | 133.59 | 147.71 | 134.36 | 145.42 |
| | 4 | 129.37 | 114.50 | 138.29 | 136.06 | 142.01 | 139.78 | 142.01 | 139.03 |
| | 5 | 72.18 | 63.26 | 120.68 | 107.95 | 120.68 | 107.20 | 120.68 | 107.95 |
| | 6 | 46.18 | 29.62 | 80.53 | 94.23 | 93.51 | 93.46 | 101.15 | 95.00 |
| | 7 | 107.39 | 57.31 | 108.95 | 105.77 | 106.61 | 101.92 | 106.61 | 102.69 |
| | 8 | 101.15 | 26.92 | 101.15 | 76.54 | 101.15 | 101.92 | 99.62 | 102.69 |
| 3 (CIA + | 1 | −8.92 | −6.74 | −5.20 | −4.49 | −5.20 | −5.24 | −6.32 | −5.99 |
| Dexamethasone) | 2 | −7.28 | −4.25 | −6.51 | −4.63 | −6.90 | −6.18 | −6.90 | −6.18 |
| | 3 | −2.17 | −1.45 | 4.33 | 4.36 | 3.61 | 4.36 | 2.89 | 3.64 |
| | 4 | −3.77 | −5.95 | −1.51 | −2.97 | −2.26 | −2.97 | −1.51 | −2.97 |
| | 5 | −3.09 | −1.56 | −0.77 | 0.00 | −1.54 | −1.56 | 0.00 | 0.78 |
| | 6 | −10.77 | −12.36 | −9.62 | −10.04 | −8.85 | −9.27 | −9.62 | −9.27 |
| | 7 | −0.75 | −2.95 | 0.00 | −1.48 | −0.75 | −2.58 | −2.25 | −2.95 |
| | 8 | −6.99 | −8.06 | −4.78 | −5.86 | −4.78 | −5.13 | −4.78 | −5.13 |
| 4 (CIA + | 1 | 60.24 | 61.02 | 113.78 | 105.91 | 112.99 | 103.54 | 112.99 | 106.69 |
| GSK620) | 2 | 53.99 | 59.00 | 92.78 | 87.36 | 92.78 | 88.12 | 90.49 | 86.59 |
| | 3 | 9.49 | 7.84 | 88.54 | 88.63 | 83.00 | 82.75 | 84.58 | 72.94 |
| | 4 | 8.79 | 76.92 | 109.89 | 130.40 | 123.81 | 130.40 | 122.34 | 125.27 |
| | 5 | 10.58 | 7.33 | 67.15 | 6.59 | 90.15 | 6.59 | 90.88 | 17.95 |
| | 6 | 19.48 | 17.36 | 53.18 | 53.96 | 63.67 | 65.66 | 65.17 | 64.15 |
| | 7 | 62.36 | 60.38 | 101.90 | 79.25 | 100.00 | 77.74 | 92.78 | 78.49 |
| | 8 | 29.63 | 33.71 | 72.96 | 75.28 | 71.48 | 74.16 | 71.48 | 74.16 |
| 5 (CIA + Example | 1 | 23.99 | 18.91 | 63.47 | 96.73 | 105.54 | 100.36 | 111.44 | 101.09 |
| 101 3 mg/kg) | 2 | 31.80 | 25.86 | 83.52 | 67.68 | 91.95 | 67.68 | 90.42 | 69.20 |
| | 3 | 28.90 | 20.97 | 101.14 | 91.39 | 116.35 | 117.60 | 128.52 | 117.60 |
| | 4 | 44.87 | 74.62 | 98.86 | 100.38 | 108.75 | 98.86 | 109.51 | 97.35 |
| | 5 | 22.68 | 27.24 | 81.04 | 76.49 | 85.50 | 76.49 | 86.25 | 74.25 |
| | 6 | 86.59 | 84.29 | 106.51 | 106.51 | 107.28 | 104.98 | 104.98 | 106.13 |
| | 7 | 50.55 | 50.19 | 80.59 | 89.22 | 90.11 | 107.81 | 93.04 | 105.58 |
| | 8 | 30.34 | 48.69 | 82.40 | 86.89 | 81.65 | 86.14 | 80.15 | 84.64 |
| 6 (CIA + Example | 1 | 53.43 | 37.01 | 72.92 | 58.36 | 71.48 | 67.62 | 71.48 | 66.90 |
| 101 10 mg/kg) | 2 | 36.90 | 45.42 | 61.90 | 65.34 | 61.51 | 63.35 | 60.71 | 62.15 |
| | 3 | 80.93 | 94.98 | 97.28 | 94.21 | 93.39 | 90.35 | 92.61 | 89.58 |
| | 4 | 93.39 | 47.10 | 109.73 | 111.20 | 105.45 | 108.88 | 105.06 | 106.95 |
| | 5 | 7.66 | 6.18 | 15.71 | 22.39 | 13.03 | 21.62 | 13.03 | 21.62 |
| | 6 | 22.69 | 10.73 | 76.54 | 46.36 | 78.08 | 42.91 | 78.85 | 64.75 |
| | 7 | 8.79 | 10.26 | 20.15 | 15.38 | 20.51 | 14.65 | 33.70 | 32.97 |
| | 8 | 19.44 | 20.72 | 19.44 | 19.92 | 26.59 | 23.90 | 26.59 | 28.69 |
| 7 (CIA + Example | 1 | 9.81 | 13.86 | 9.81 | 12.73 | 6.79 | 7.49 | 7.55 | 6.74 |
| 101 30 mg/kg) | 2 | 35.74 | 4.98 | 55.51 | 42.91 | 55.13 | 49.81 | 57.79 | 60.54 |
| | 3 | 18.11 | 28.35 | 43.02 | 42.53 | 55.09 | 63.60 | 58.87 | 65.13 |
| | 4 | 4.56 | 3.66 | 11.41 | 2.90 | 18.25 | 4.41 | 16.73 | 5.16 |
| | 5 | 4.56 | 14.23 | 11.41 | 13.46 | 17.49 | 13.46 | 16.73 | 12.69 |
| | 6 | 1.91 | 3.42 | 28.24 | 0.00 | 41.22 | 38.78 | 56.87 | 52.09 |
| | 7 | 10.81 | 31.56 | 48.65 | 48.29 | 49.42 | 55.13 | 64.09 | 61.22 |
| | 8 | −1.87 | −3.00 | 26.97 | 25.47 | 24.72 | 26.22 | 24.72 | 25.09 |

TABLE 14C
Mean animal paw volume (mL) ± SD

| | Day 0 | | Day 6 | | Day 12 | | Day 13 | | Day 14 | |
|---|---|---|---|---|---|---|---|---|---|---|
| Group | L | R | L | R | L | R | L | R | L | R |
| 1 (No CIA + | 1.31 ± | 1.31 ± | 1.33 ± | 1.33 ± | 1.38 ± | 1.39 ± | 1.39 ± | 1.41 ± | 1.41 ± | 1.42 ± |
| Vehicle) | 0.03 | 0.02 | 0.02 | 0.02 | 0.03 | 0.04 | 0.04 | 0.04 | 0.05 | 0.04 |
| 2 (CIA + | 1.32 ± | 1.32 ± | 1.34 ± | 1.34 ± | 1.59 ± | 1.55 ± | 1.73 ± | 1.61 ± | 2.02 ± | 1.87 ± |
| Vehicle) | 0.02 | 0.02 | 0.03 | 0.03 | 0.26 | 0.19 | 0.36 | 0.23 | 0.45 | 0.43 |
| 3 (CIA + | 1.33 ± | 1.33 ± | 1.29 ± | 1.29 ± | 1.27 ± | 1.26 ± | 1.28 ± | 1.28 ± | 1.27 ± | 1.26 ± |
| Dex) | 0.03 | 0.04 | 0.03 | 0.02 | 0.06 | 0.05 | 0.05 | 0.05 | 0.05* | 0.05 |
| 4 (CIA + | 1.32 ± | 1.32 ± | 1.34 ± | 1.34 ± | 1.36 ± | 1.37 ± | 1.38 ± | 1.41 ± | 1.55 ± | 1.67 ± |
| GSK620) | 0.04 | 0.04 | 0.03 | 0.04 | 0.03 | 0.03 | 0.04 | 0.07 | 0.17 | 0.28 |
| 5 (CIA + Example | 1.33 ± | 1.33 ± | 1.34 ± | 1.35 ± | 1.41 ± | 1.40 ± | 1.53 ± | 1.46 ± | 1.69 ± | 1.59 ± |
| 101 3 mg/kg) | 0.02 | 0.02 | 0.03 | 0.03 | 0.09 | 0.03 | 0.15 | 0.10 | 0.13 | 0.23 |
| 6 (CIA + Example | 1.31 ± | 1.31 ± | 1.32 ± | 1.32 ± | 1.41 ± | 1.41 ± | 1.52 ± | 1.45 ± | 1.70 ± | 1.59 ± |
| 101 10 mg/kg) | 0.05 | 0.05 | 0.06 | 0.05 | 0.12 | 0.19 | 0.22 | 0.19 | 0.42 | 0.30 |
| 7 (CIA + Example | 1.32 ± | 1.32 ± | 1.33 ± | 1.33 ± | 1.33 ± | 1.36 ± | 1.35 ± | 1.37 ± | 1.35 ± | 1.37 ± |
| 101 30 mg/kg) | 0.01 | 0.01 | 0.01 | 0.01 | 0.02 | 0.06 | 0.02 | 0.07 | 0.03 | 0.07 |

| | Day 15 | | Day 18 | | Day 19 | | Day 21 | |
|---|---|---|---|---|---|---|---|---|
| Group | L | R | L | R | L | R | L | R |
| 1 (No CIA + | 1.41 ± | 1.43 ± | 1.42 ± | 1.42 ± | 1.41 ± | 1.41 ± | 1.41 ± | 1.41 ± |
| Vehicle) | 0.05* | 0.04* | 0.04** | 0.04** | 0.04** | 0.04** | 0.05** | 0.05** |
| 2 (CIA + | 2.39 ± | 2.15 ± | 2.77 ± | 2.75 ± | 2.83 ± | 2.84 ± | 2.86 ± | 2.84 ± |
| Vehicle) | 0.46 | 0.41 | 0.25 | 0.27 | 0.24 | 0.28 | 0.24 | 0.27 |
| 3 (CIA + | 1.26 ± | 1.26 ± | 1.29 ± | 1.29 ± | 1.29 ± | 1.28 ± | 1.28 ± | 1.29 ± |
| Dex) | 0.06** | 0.06* | 0.08** | 0.08** | 0.07** | 0.08** | 0.07** | 0.07** |
| 4 (CIA + | 1.74 ± | 1.86 ± | 2.48 ± | 2.35 ± | 2.54 ± | 2.36 ± | 2.53 ± | 2.35 ± |
| GSK620) | 0.29 | 0.37 | 0.27 | 0.48 | 0.28 | 0.47 | 0.26 | 0.42 |
| 5 (CIA + Example | 1.86 ± | 1.92 ± | 2.49 ± | 2.53 ± | 2.64 ± | 2.60 ± | 2.67 ± | 2.59 ± |
| 101 3 mg/kg) | 0.27 | 0.31 | 0.16 | 0.17 | 0.15 | 0.23 | 0.20 | 0.23 |
| 6 (CIA + Example | 1.83 ± | 1.75 ± | 2.08 ± | 2.02 ± | 2.07 ± | 2.02 ± | 2.09 ± | 2.08 ± |
| 101 10 mg/kg) | 0.42 | 0.38 | 0.48 | 0.46 | 0.45* | 0.45* | 0.43* | 0.40* |
| 7 (CIA + Example | 1.45 ± | 1.48 ± | 1.70 ± | 1.63 ± | 1.76 ± | 1.74 ± | 1.82 ± | 1.79 ± |
| 101 30 mg/kg) | 0.16* | 0.16 | 0.23** | 0.25** | 0.25** | 0.29** | 0.30** | 0.34** |

One-way ANOVA comparison to Group 2;
*p < 0.05;
**p < 0.01

TABLE 14D
Average animal paw volume percent change (%)

| | Day 0 | | Day 6 | | Day 12 | | Day 13 | | Day 14 | |
|---|---|---|---|---|---|---|---|---|---|---|
| Group | L | R | L | R | L | R | L | R | L | R |
| 1 (No CIA + | 0.00 | 0.00 | 1.49 | 1.15 | 5.79 | 6.25 | 6.51 | 7.39 | 7.46 | 8.40 |
| Vehicle) | | | | | | | | | | |
| 2 (CIA + | 0.00 | 0.00 | 1.70 | 1.70 | 20.65 | 17.69 | 31.35 | 21.69 | 53.30 | 41.66 |
| Vehicle) | | | | | | | | | | |
| 3 (CIA + | 0.00 | 0.00 | −3.37 | −3.26 | −4.94 | −5.21 | −3.57 | −4.08 | −4.60 | −5.07 |
| Dex) | | | | | | | | | | |
| 4 (CIA + | 0.00 | 0.00 | 1.04 | 1.50 | 3.13 | 3.69 | 4.09 | 6.88 | 17.19 | 26.62 |
| GSK620) | | | | | | | | | | |
| 5 (CIA + Example | 0.00 | 0.00 | 1.09 | 1.23 | 5.80 | 5.01 | 15.11 | 9.28 | 27.04 | 19.52 |
| 101 3 mg/kg) | | | | | | | | | | |
| 6 (CIA + Example | 0.00 | 0.00 | 1.14 | 0.87 | 7.61 | 7.98 | 16.61 | 10.89 | 30.15 | 21.45 |
| 101 10 mg/kg) | | | | | | | | | | |
| 7 (CIA + Example | 0.00 | 0.00 | 0.76 | 0.75 | 1.33 | 2.87 | 2.76 | 4.15 | 2.39 | 3.83 |
| 101 30 mg/kg) | | | | | | | | | | |

| | Day 15 | | Day 18 | | Day 19 | | Day 21 | |
|---|---|---|---|---|---|---|---|---|
| Group | L | R | L | R | L | R | L | R |
| 1 (No CIA + | 7.88 | 8.77 | 8.17 | 8.11 | 7.69 | 7.82 | 8.07 | 7.64 |
| Vehicle) | | | | | | | | |
| 2 (CIA + | 80.96 | 62.92 | 109.98 | 108.54 | 115.06 | 115.31 | 116.76 | 115.28 |
| Vehicle) | | | | | | | | |
| 3 (CIA + | −5.47 | −5.41 | −3.01 | −3.14 | −3.33 | −3.57 | −3.56 | −3.51 |
| Dex) | | | | | | | | |
| 4 (CIA + | 31.82 | 40.45 | 87.52 | 78.42 | 92.23 | 78.62 | 91.34 | 78.28 |
| GSK620) | | | | | | | | |
| 5 (CIA + Example | 39.96 | 43.85 | 87.19 | 89.41 | 98.39 | 94.99 | 100.54 | 94.48 |
| 101 3 mg/kg) | | | | | | | | |
| 6 (CIA + Example | 40.41 | 34.05 | 59.21 | 54.15 | 58.75 | 54.16 | 60.25 | 59.20 |
| 101 10 mg/kg) | | | | | | | | |
| 7 (CIA + Example | 10.45 | 12.13 | 29.38 | 23.54 | 33.52 | 32.36 | 37.92 | 36.08 |
| 101 30 mg/kg) | | | | | | | | |

TABLE 15A
Detailed Clinical Scores

| | | Day 0 | | | | | Day 6 | | | | | Day 12 | | | | |
|---|---|---|---|---|---|---|---|---|---|---|---|---|---|---|---|---|
| | | | | left | right | | | | left | right | | | | left | right | |
| Table | | left | right | hind | hind | | left | right | hind | hind | | left | right | hind | hind | |
| Group | ID | forelimb | forelimb | limb | limb | score | forelimb | forelimb | limb | limb | score | forelimb | forelimb | limb | limb | score |
| 1 (No CIA + | 1 | 0 | 0 | 0 | 0 | 0 | 0 | 0 | 0 | 0 | 0 | 0 | 0 | 0 | 0 | 0 |
| Vehicle) | 2 | 0 | 0 | 0 | 0 | 0 | 0 | 0 | 0 | 0 | 0 | 0 | 0 | 0 | 0 | 0 |
| | 3 | 0 | 0 | 0 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 2 (CIA + | 1 | 0 | 0 | 0 | 0 | 0 | 0 | 0 | 0 | 0 | 0 | 0 | 0 | 2 | 2 | 4 |
| Vehicle) | 2 | 0 | 0 | 0 | 0 | 0 | 0 | 0 | 0 | 0 | 0 | 0 | 0 | 0 | 0 | 0 |
| | 3 | 0 | 0 | 0 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 1 | 0 | 3 | 2 | 6 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 3 (CIA + | 1 | 0 | 0 | 0 | 0 | 0 | 0 | 0 | 0 | 0 | 0 | 0 | 0 | 0 | 0 | 0 |
| Dexamethasone) | 2 | 0 | 0 | 0 | 0 | 0 | 0 | 0 | 0 | 0 | 0 | 0 | 0 | 0 | 0 | 0 |
| | 3 | 0 | 0 | 0 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 4 (CIA + | 1 | 0 | 0 | 0 | 0 | 0 | 0 | 0 | 0 | 0 | 0 | 0 | 0 | 0 | 0 | 0 |
| GSK620) | 2 | 0 | 0 | 0 | 0 | 0 | 0 | 0 | 0 | 0 | 0 | 0 | 0 | 0 | 0 | 0 |
| | 3 | 0 | 0 | 0 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 5 (CIA + Example | 1 | 0 | 0 | 0 | 0 | 0 | 0 | 0 | 0 | 0 | 0 | 0 | 0 | 0 | 0 | 0 |
| 101 3 mg/kg) | 2 | 0 | 0 | 0 | 0 | 0 | 0 | 0 | 0 | 0 | 0 | 0 | 0 | 0 | 0 | 0 |
| | 3 | 0 | 0 | 0 | 0 | 0 | 0 | 0 | 0 | 0 | 0 | 0 | 1 | 0 | 0 | 1 |
| | 4 | 0 | 0 | 0 | 0 | 0 | 0 | 0 | 0 | 0 | 0 | 0 | 1 | 0 | 0 | 1 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 1 | 0 | 1 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 6 (CIA + Example | 1 | 0 | 0 | 0 | 0 | 0 | 0 | 0 | 0 | 0 | 0 | 0 | 0 | 1 | 0 | 1 |
| 101 10 mg/kg) | 2 | 0 | 0 | 0 | 0 | 0 | 0 | 0 | 0 | 0 | 0 | 0 | 0 | 0 | 0 | 0 |
| | 3 | 0 | 0 | 0 | 0 | 0 | 0 | 0 | 0 | 0 | 0 | 0 | 0 | 0 | 2 | 2 |
| | 4 | 0 | 0 | 0 | 0 | 0 | 0 | 0 | 0 | 0 | 0 | 0 | 0 | 1 | 0 | 1 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 7 (CIA + Example | 1 | 0 | 0 | 0 | 0 | 0 | 0 | 0 | 0 | 0 | 0 | 0 | 0 | 0 | 1 | 1 |
| 101 30 mg/kg) | 2 | 0 | 0 | 0 | 0 | 0 | 0 | 0 | 0 | 0 | 0 | 0 | 0 | 0 | 0 | 0 |
| | 3 | 0 | 0 | 0 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| | | Day 13 | | | | | Day 14 | | | | | Day 15 | | | | |
| | | | | left | right | | | | left | right | | | | left | right | |
| | | left | right | hind | hind | | left | right | hind | hind | | left | right | hind | hind | |
| Group | ID | forelimb | forelimb | limb | limb | score | forelimb | forelimb | limb | limb | score | forelimb | forelimb | limb | limb | score |
| 1 (No CIA + | 1 | 0 | 0 | 0 | 0 | 0 | 0 | 0 | 0 | 0 | 0 | 0 | 0 | 0 | 0 | 0 |
| Vehicle) | 2 | 0 | 0 | 0 | 0 | 0 | 0 | 0 | 0 | 0 | 0 | 0 | 0 | 0 | 0 | 0 |
| | 3 | 0 | 0 | 0 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 2 (CIA + | 1 | 0 | 0 | 3 | 2 | 5 | 0 | 0 | 4 | 4 | 8 | 0 | 1 | 4 | 4 | 9 |
| Vehicle) | 2 | 0 | 0 | 0 | 0 | 0 | 0 | 0 | 1 | 2 | 3 | 0 | 0 | 3 | 3 | 6 |
| | 3 | 0 | 0 | 0 | 0 | 0 | 0 | 0 | 0 | 0 | 0 | 0 | 0 | 1 | 3 | 4 |
| | 4 | 2 | 1 | 4 | 3 | 10 | 3 | 2 | 4 | 4 | 13 | 4 | 4 | 4 | 4 | 16 |
| | 5 | 0 | 0 | 0 | 0 | 0 | 1 | 0 | 2 | 1 | 4 | 1 | 1 | 3 | 3 | 8 |
| | 6 | 0 | 0 | 0 | 0 | 0 | 0 | 0 | 0 | 0 | 0 | 0 | 0 | 2 | 1 | 3 |
| | 7 | 0 | 0 | 2 | 1 | 3 | 0 | 0 | 3 | 2 | 5 | 0 | 0 | 4 | 3 | 7 |
| | 8 | 0 | 0 | 1 | 0 | 1 | 0 | 0 | 3 | 0 | 3 | 0 | 0 | 4 | 0 | 4 |
| 3 (CIA + | 1 | 0 | 0 | 0 | 0 | 0 | 0 | 0 | 0 | 0 | 0 | 0 | 0 | 0 | 0 | 0 |
| Dexamethasone) | 2 | 0 | 0 | 0 | 0 | 0 | 0 | 0 | 0 | 0 | 0 | 0 | 0 | 0 | 0 | 0 |
| | 3 | 0 | 0 | 0 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 4 (CIA + | 1 | 0 | 0 | 0 | 0 | 0 | 0 | 0 | 1 | 2 | 3 | 0 | 0 | 3 | 3 | 6 |
| GSK620) | 2 | 0 | 0 | 0 | 0 | 0 | 0 | 0 | 2 | 0 | 2 | 0 | 0 | 3 | 3 | 6 |
| | 3 | 0 | 0 | 0 | 0 | 0 | 0 | 0 | 0 | 0 | 0 | 0 | 0 | 0 | 0 | 0 |
| | 4 | 0 | 0 | 0 | 1 | 1 | 0 | 0 | 0 | 3 | 3 | 0 | 0 | 0 | 4 | 4 |
| | 5 | 0 | 0 | 0 | 0 | 0 | 0 | 0 | 0 | 0 | 0 | 0 | 0 | 1 | 0 | 1 |
| | 6 | 0 | 0 | 0 | 0 | 0 | 0 | 0 | 0 | 0 | 0 | 0 | 0 | 1 | 1 | 2 |
| | 7 | 0 | 0 | 0 | 0 | 0 | 0 | 0 | 2 | 2 | 4 | 0 | 0 | 3 | 3 | 6 |
| | 8 | 0 | 0 | 0 | 0 | 0 | 0 | 0 | 2 | 2 | 4 | 0 | 0 | 2 | 2 | 4 |
| 5 (CIA + Example | 1 | 0 | 0 | 1 | 0 | 1 | 0 | 0 | 1 | 1 | 2 | 0 | 0 | 1 | 1 | 2 |
| 101 3 mg/kg) | 2 | 0 | 0 | 1 | 0 | 1 | 0 | 0 | 1 | 1 | 2 | 0 | 0 | 2 | 1 | 3 |
| | 3 | 0 | 1 | 1 | 0 | 2 | 0 | 1 | 1 | 0 | 2 | 0 | 1 | 1 | 1 | 3 |
| | 4 | 0 | 1 | 1 | 0 | 2 | 0 | 1 | 1 | 2 | 4 | 0 | 1 | 2 | 3 | 6 |
| | 5 | 0 | 0 | 0 | 0 | 0 | 0 | 0 | 1 | 0 | 1 | 0 | 0 | 1 | 2 | 3 |
| | 6 | 0 | 0 | 2 | 1 | 3 | 0 | 0 | 3 | 3 | 6 | 0 | 0 | 4 | 4 | 8 |
| | 7 | 0 | 0 | 0 | 0 | 0 | 0 | 0 | 1 | 0 | 1 | 0 | 0 | 3 | 3 | 6 |
| | 8 | 0 | 0 | 0 | 0 | 0 | 0 | 0 | 1 | 0 | 1 | 0 | 0 | 2 | 2 | 4 |
| 6 (CIA + Example | 1 | 0 | 0 | 2 | 1 | 3 | 0 | 0 | 3 | 1 | 4 | 0 | 0 | 3 | 2 | 5 |
| 101 10 mg/kg) | 2 | 0 | 0 | 0 | 0 | 0 | 0 | 0 | 3 | 1 | 4 | 0 | 0 | 2 | 2 | 4 |
| | 3 | 0 | 0 | 2 | 2 | 4 | 0 | 0 | 3 | 3 | 6 | 0 | 0 | 3 | 4 | 7 |
| | 4 | 0 | 0 | 2 | 0 | 2 | 0 | 0 | 4 | 1 | 5 | 0 | 0 | 4 | 2 | 6 |
| | 5 | 0 | 0 | 0 | 0 | 0 | 0 | 0 | 0 | 0 | 0 | 0 | 0 | 0 | 0 | 0 |
| | 6 | 0 | 0 | 1 | 0 | 1 | 0 | 0 | 0 | 0 | 0 | 0 | 0 | 1 | 0 | 1 |
| | 7 | 0 | 0 | 0 | 0 | 0 | 0 | 0 | 0 | 0 | 0 | 0 | 0 | 0 | 1 | 1 |
| | 8 | 0 | 0 | 0 | 0 | 0 | 0 | 0 | 0 | 0 | 0 | 0 | 0 | 1 | 1 | 2 |
| 7 (CIA + Example | 1 | 0 | 0 | 0 | 1 | 1 | 0 | 0 | 0 | 1 | 1 | 0 | 0 | 0 | 1 | 1 |
| 101 30 mg/kg) | 2 | 0 | 0 | 0 | 0 | 0 | 0 | 0 | 0 | 0 | 0 | 0 | 0 | 2 | 0 | 2 |
| | 3 | 0 | 0 | 0 | 0 | 0 | 0 | 0 | 0 | 0 | 0 | 0 | 0 | 1 | 1 | 2 |
| | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 2 | 2 |
| | 8 | 0 | 0 | 0 | 0 | 0 | 0 | 0 | 0 | 0 | 0 | 0 | 0 | 0 | 0 | 0 |
| | | Day 18 | | | | | Day 19 | | | | | Day 21 | | | | |
| | | | | left | right | | | | left | right | | | | left | right | |
| | | left | right | hind | hind | | left | right | hind | hind | | left | right | hind | hind | |
| Group | ID | forelimb | forelimb | limb | limb | score | forelimb | forelimb | limb | limb | score | forelimb | forelimb | limb | limb | score |
| 1 (No CIA + | 1 | 0 | 0 | 0 | 0 | 0 | 0 | 0 | 0 | 0 | 0 | 0 | 0 | 0 | 0 | 0 |
| Vehicle) | 2 | 0 | 0 | 0 | 0 | 0 | 0 | 0 | 0 | 0 | 0 | 0 | 0 | 0 | 0 | 0 |
| | 3 | 0 | 0 | 0 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 2 (CIA + | 1 | 4 | 4 | 4 | 4 | 16 | 4 | 4 | 4 | 4 | 16 | 4 | 4 | 4 | 4 | 16 |
| Vehicle) | 2 | 1 | 1 | 4 | 4 | 10 | 1 | 1 | 4 | 4 | 10 | 2 | 1 | 4 | 4 | 11 |
| | 3 | 4 | 4 | 4 | 4 | 16 | 4 | 4 | 4 | 4 | 16 | 4 | 4 | 4 | 4 | 16 |
| | 4 | 4 | 4 | 4 | 4 | 16 | 4 | 4 | 4 | 4 | 16 | 4 | 4 | 4 | 4 | 16 |
| | 5 | 1 | 2 | 4 | 4 | 11 | 1 | 2 | 4 | 4 | 11 | 1 | 2 | 4 | 4 | 11 |
| | 6 | 1 | 1 | 4 | 4 | 10 | 1 | 1 | 4 | 4 | 10 | 1 | 1 | 4 | 4 | 10 |
| | 7 | 1 | 1 | 4 | 4 | 10 | 1 | 1 | 4 | 4 | 10 | 1 | 1 | 4 | 4 | 10 |
| | 8 | 1 | 1 | 4 | 4 | 10 | 1 | 1 | 4 | 4 | 10 | 1 | 1 | 4 | 4 | 10 |
| 3 (CIA + | 1 | 0 | 0 | 0 | 0 | 0 | 0 | 0 | 0 | 0 | 0 | 0 | 0 | 0 | 0 | 0 |
| Dexamethasone) | 2 | 0 | 0 | 0 | 0 | 0 | 0 | 0 | 0 | 0 | 0 | 0 | 0 | 0 | 0 | 0 |
| | 3 | 0 | 0 | 0 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 | 0 | 0 | 0 |
| | 5 | 0 | 0 | 0 | 0 | 0 | 0 | 0 | 0 | 0 | 0 | 0 | 0 | 0 | 0 | 0 |
| | 6 | 0 | 0 | 0 | 0 | 0 | 0 | 0 | 0 | 0 | 0 | 0 | 0 | 0 | 0 | 0 |
| | 7 | 0 | 0 | 0 | 0 | 0 | 0 | 0 | 0 | 0 | 0 | 0 | 0 | 0 | 0 | 0 |
| | 8 | 0 | 0 | 0 | 0 | 0 | 0 | 0 | 0 | 0 | 0 | 0 | 0 | 0 | 0 | 0 |
| 4 (CIA + | 1 | 0 | 0 | 4 | 4 | 8 | 1 | 0 | 4 | 4 | 9 | 1 | 0 | 4 | 4 | 9 |
| GSK620) | 2 | 0 | 0 | 4 | 4 | 8 | 1 | 1 | 4 | 4 | 10 | 2 | 1 | 4 | 4 | 11 |
| | 3 | 0 | 0 | 4 | 4 | 8 | 0 | 0 | 3 | 3 | 6 | 0 | 0 | 3 | 3 | 6 |
| | 4 | 0 | 0 | 4 | 4 | 8 | 1 | 0 | 4 | 4 | 9 | 1 | 0 | 4 | 4 | 9 |
| | 5 | 0 | 0 | 4 | 0 | 4 | 1 | 0 | 4 | 0 | 5 | 1 | 0 | 4 | 1 | 6 |
| | 6 | 0 | 0 | 3 | 3 | 6 | 0 | 1 | 3 | 3 | 7 | 0 | 1 | 3 | 3 | 7 |
| | 7 | 0 | 0 | 4 | 4 | 8 | 1 | 0 | 4 | 3 | 8 | 1 | 0 | 4 | 3 | 8 |
| | 8 | 0 | 0 | 4 | 4 | 8 | 1 | 0 | 3 | 3 | 7 | 1 | 0 | 3 | 3 | 7 |
| 5 (CIA + Example | 1 | 0 | 0 | 3 | 4 | 7 | 1 | 0 | 4 | 4 | 9 | 1 | 0 | 4 | 4 | 9 |
| 101 3 mg/kg) | 2 | 0 | 0 | 4 | 3 | 7 | 0 | 0 | 4 | 3 | 7 | 0 | 0 | 4 | 3 | 7 |
| | 3 | 0 | 0 | 4 | 4 | 8 | 0 | 0 | 4 | 4 | 8 | 0 | 0 | 4 | 4 | 8 |
| | 4 | 0 | 0 | 4 | 4 | 8 | 4 | 4 | 4 | 4 | 16 | 3 | 3 | 4 | 4 | 14 |
| | 5 | 0 | 0 | 4 | 4 | 8 | 0 | 0 | 4 | 3 | 7 | 0 | 0 | 4 | 3 | 7 |
| | 6 | 4 | 0 | 4 | 4 | 12 | 4 | 0 | 4 | 4 | 12 | 4 | 1 | 4 | 4 | 13 |
| | 7 | 0 | 4 | 4 | 4 | 12 | 0 | 4 | 4 | 4 | 12 | 0 | 4 | 4 | 4 | 12 |
| | 8 | 0 | 0 | 4 | 4 | 8 | 0 | 0 | 4 | 4 | 8 | 0 | 0 | 4 | 4 | 8 |
| 6 (CIA + Example | 1 | 0 | 0 | 4 | 3 | 7 | 0 | 1 | 3 | 3 | 7 | 0 | 1 | 3 | 3 | 7 |
| 101 10 mg/kg) | 2 | 0 | 0 | 3 | 3 | 6 | 0 | 0 | 3 | 3 | 6 | 0 | 0 | 3 | 3 | 6 |
| | 3 | 0 | 4 | 4 | 4 | 12 | 0 | 4 | 4 | 4 | 12 | 0 | 4 | 4 | 4 | 12 |
| | 4 | 0 | 3 | 4 | 4 | 11 | 0 | 3 | 4 | 4 | 11 | 0 | 4 | 4 | 4 | 12 |
| | 5 | 0 | 0 | 0 | 1 | 1 | 0 | 0 | 0 | 2 | 2 | 0 | 0 | 0 | 2 | 2 |
| | 6 | 0 | 0 | 4 | 2 | 6 | 0 | 1 | 3 | 3 | 7 | 0 | 1 | 3 | 3 | 7 |
| | 7 | 0 | 0 | 1 | 1 | 2 | 0 | 0 | 1 | 2 | 3 | 0 | 0 | 2 | 2 | 4 |
| | 8 | 0 | 0 | 0 | 0 | 0 | 0 | 0 | 1 | 2 | 3 | 0 | 0 | 1 | 1 | 2 |
| 7 (CIA + Example | 1 | 0 | 0 | 0 | 0 | 0 | 0 | 0 | 0 | 0 | 0 | 0 | 0 | 0 | 0 | 0 |
| 101 30 mg/kg) | 2 | 0 | 0 | 3 | 2 | 5 | 0 | 0 | 3 | 3 | 6 | 0 | 0 | 3 | 3 | 6 |
| | 3 | 0 | 0 | 2 | 2 | 4 | 0 | 1 | 3 | 3 | 7 | 0 | 1 | 3 | 3 | 7 |
| | 4 | 0 | 0 | 0 | 0 | 0 | 0 | 0 | 1 | 0 | 1 | 0 | 0 | 1 | 0 | 1 |
| | 5 | 0 | 0 | 0 | 0 | 0 | 0 | 0 | 1 | 0 | 1 | 0 | 0 | 1 | 0 | 1 |
| | 6 | 0 | 0 | 1 | 0 | 1 | 0 | 1 | 2 | 3 | 6 | 0 | 0 | 3 | 3 | 6 |
| | 7 | 0 | 0 | 2 | 2 | 4 | 0 | 0 | 2 | 3 | 5 | 0 | 0 | 3 | 3 | 6 |
| | 8 | 0 | 0 | 1 | 1 | 2 | 0 | 0 | 1 | 2 | 3 | 0 | 0 | 1 | 1 | 2 |

TABLE 15B
Average detailed clinical score

| | Day 0 | | | | | Day 6 | | | | | Day 12 | | | | |
|---|---|---|---|---|---|---|---|---|---|---|---|---|---|---|---|
| | left | right | left | right | | left | right | left | right | | left | right | left | right | |
| | fore- | fore- | hind | hind | | fore- | fore- | hind | hind | | fore- | fore- | hind | hind | |
| Group | limb | limb | limb | limb | score | limb | limb | limb | limb | score | limb | limb | limb | limb | score |
| 1 (No CIA + | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| Vehicle) | | | | | | | | | | | | | | | |
| 2 (CIA + | 0.00 | 0.00 | 0.00 | 0.00 | 0.00 | 0.00 | 0.00 | 0.00 | 0.00 | 0.00 | 0.13 | 0.00 | 0.63 | 0.50 | 1.25 |
| Vehicle) | | | | | | | | | | | | | | | |
| 3 (CIA + | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| Dex) | | | | | | | | | | | | | | | |
| 4 (CIA + | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| GSK620) | | | | | | | | | | | | | | | |
| 5 (CIA + Example | 0.00 | 0.00 | 0.00 | 0.00 | 0.00 | 0.00 | 0.00 | 0.00 | 0.00 | 0.00 | 0.00 | 0.25 | 0.13 | 0.00 | 0.38 |
| 101 3 mg/kg) | | | | | | | | | | | | | | | |
| 6 (CIA + Example | 0.00 | 0.00 | 0.00 | 0.00 | 0.00 | 0.00 | 0.00 | 0.00 | 0.00 | 0.00 | 0.00 | 0.00 | 0.25 | 0.25 | 0.50 |
| 101 10 mg/kg) | | | | | | | | | | | | | | | |
| 7 (CIA + Example | 0.00 | 0.00 | 0.00 | 0.00 | 0.00 | 0.00 | 0.00 | 0.00 | 0.00 | 0.00 | 0.00 | 0.00 | 0.00 | 0.13 | 0.13 |
| 101 30 mg/kg) | | | | | | | | | | | | | | | |
| | Day 13 | | | | | Day 14 | | | | | Day 15 | | | | |
| | left | right | left | right | | left | right | left | right | | left | right | left | right | |
| | fore- | fore- | hind | hind | | fore- | fore- | hind | hind | | fore- | fore- | hind | hind | |
| Group | limb | limb | limb | limb | score | limb | limb | limb | limb | score | limb | limb | limb | limb | score |
| 1 (No CIA + | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| Vehicle) | | | | | | | | | | | | | | | |
| 2 (CIA + | 0.25 | 0.13 | 1.25 | 0.75 | 2.38 | 0.50 | 0.25 | 2.13 | 1.63 | 4.50 | 0.63 | 0.75 | 3.13 | 2.63 | 7.13 |
| Vehicle) | | | | | | | | | | | | | | | |
| 3 (CIA + | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| Dex) | | | | | | | | | | | | | | | |
| 4 (CIA + | 0.00 | 0.00 | 0.00 | 0.13 | 0.13 | 0.00 | 0.00 | 0.88 | 1.13 | 2.00 | 0.00 | 0.00 | 1.63 | 2.00 | 3.63 |
| GSK620) | | | | | | | | | | | | | | | |
| 5 (CIA + Example | 0.00 | 0.25 | 0.75 | 0.13 | 1.13 | 0.00 | 0.25 | 1.25 | 0.88 | 2.38 | 0.00 | 0.25 | 2.00 | 2.13 | 4.38 |
| 101 3 mg/kg) | | | | | | | | | | | | | | | |
| 6 (CIA + Example | 0.00 | 0.00 | 0.88 | 0.38 | 1.25 | 0.00 | 0.00 | 1.63 | 0.75 | 2.38 | 0.00 | 0.00 | 1.75 | 1.50 | 3.25 |
| 101 10 mg/kg) | | | | | | | | | | | | | | | |
| 7 (CIA + Example | 0.00 | 0.00 | 0.00 | 0.13 | 0.13 | 0.00 | 0.00 | 0.00 | 0.13 | 0.13 | 0.00 | 0.00 | 0.38 | 0.50 | 0.88 |
| 101 30 mg/kg) | | | | | | | | | | | | | | | |
| | Day 18 | | | | | Day 19 | | | | | Day 21 | | | | |
| | left | right | left | right | | left | right | left | right | | left | right | left | right | |
| | fore- | fore- | hind | hind | | fore- | fore- | hind | hind | | fore- | fore- | hind | hind | |
| Group | limb | limb | limb | limb | score | limb | limb | limb | limb | score | limb | limb | limb | limb | score |
| 1 (No CIA + | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| Vehicle) | | | | | | | | | | | | | | | |
| 2 (CIA + | 2.13 | 2.25 | 4.00 | 4.00 | 12.38 | 2.13 | 2.25 | 4.00 | 4.00 | 12.38 | 2.25 | 2.25 | 4.00 | 4.00 | 12.50 |
| Vehicle) | | | | | | | | | | | | | | | |
| 3 (CIA + | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| Dex) | | | | | | | | | | | | | | | |
| 4 (CIA + | 0.00 | 0.00 | 3.88 | 3.38 | 7.25 | 0.75 | 0.25 | 3.63 | 3.00 | 7.63 | 0.88 | 0.25 | 3.63 | 3.13 | 7.88 |
| GSK620) | | | | | | | | | | | | | | | |
| 5 (CIA + Example | 0.50 | 0.50 | 3.88 | 3.88 | 8.75 | 1.13 | 1.00 | 4.00 | 3.75 | 9.88 | 1.00 | 1.00 | 4.00 | 3.75 | 9.75 |
| 101 3 mg/kg) | | | | | | | | | | | | | | | |
| 6 (CIA + Example | 0.00 | 0.88 | 2.50 | 2.25 | 5.63 | 0.00 | 1.13 | 2.38 | 2.88 | 6.38 | 0.00 | 1.25 | 2.50 | 2.75 | 6.50 |
| 101 10 mg/kg) | | | | | | | | | | | | | | | |
| 7 (CIA + Example | 0.00 | 0.00 | 1.13 | 0.88 | 2.00 | 0.00 | 0.25 | 1.63 | 1.75 | 3.63 | 0.00 | 0.13 | 1.88 | 1.63 | 3.63 |
| 101 30 mg/kg) | | | | | | | | | | | | | | | |

TABLE 15C
Overall clinical score

| | | Overall Clinical Score | | | | | | | | |
|---|---|---|---|---|---|---|---|---|---|---|
| Group | # | Day 0 | Day 6 | Day 12 | Day 13 | Day 14 | Day 15 | Day 18 | Day 19 | Day 21 |
| 1 (No CIA + | 1 | 0 | 0 | 0 | 0 | 0 | 0 | 0 | 0 | 0 |
| Vehicle) | 2 | 0 | 0 | 0 | 0 | 0 | 0 | 0 | 0 | 0 |
| | 3 | 0 | 0 | 0 | 0 | 0 | 0 | 0 | 0 | 0 |
| | 4 | 0 | 0 | 0 | 0 | 0 | 0 | 0 | 0 | 0 |
| | 5 | 0 | 0 | 0 | 0 | 0 | 0 | 0 | 0 | 0 |
| | 6 | 0 | 0 | 0 | 0 | 0 | 0 | 0 | 0 | 0 |
| | 7 | 0 | 0 | 0 | 0 | 0 | 0 | 0 | 0 | 0 |
| | 8 | 0 | 0 | 0 | 0 | 0 | 0 | 0 | 0 | 0 |
| 2 (CIA + | 1 | 0 | 0 | 4 | 5 | 8 | 9 | 16 | 16 | 16 |
| Vehicle) | 2 | 0 | 0 | 0 | 0 | 3 | 6 | 10 | 10 | 11 |
| | 3 | 0 | 0 | 0 | 0 | 0 | 4 | 16 | 16 | 16 |
| | 4 | 0 | 0 | 6 | 10 | 13 | 16 | 16 | 16 | 16 |
| | 5 | 0 | 0 | 0 | 0 | 4 | 8 | 11 | 11 | 11 |
| | 6 | 0 | 0 | 0 | 0 | 0 | 3 | 10 | 10 | 10 |
| | 7 | 0 | 0 | 0 | 3 | 5 | 7 | 10 | 10 | 10 |
| | 8 | 0 | 0 | 0 | 1 | 3 | 4 | 10 | 10 | 10 |
| 3 (CIA + | 1 | 0 | 0 | 0 | 0 | 0 | 0 | 0 | 0 | 0 |
| Dexamethasone) | 2 | 0 | 0 | 0 | 0 | 0 | 0 | 0 | 0 | 0 |
| | 3 | 0 | 0 | 0 | 0 | 0 | 0 | 0 | 0 | 0 |
| | 4 | 0 | 0 | 0 | 0 | 0 | 0 | 0 | 0 | 0 |
| | 5 | 0 | 0 | 0 | 0 | 0 | 0 | 0 | 0 | 0 |
| | 6 | 0 | 0 | 0 | 0 | 0 | 0 | 0 | 0 | 0 |
| | 7 | 0 | 0 | 0 | 0 | 0 | 0 | 0 | 0 | 0 |
| | 8 | 0 | 0 | 0 | 0 | 0 | 0 | 0 | 0 | 0 |
| 4 (CIA + | 1 | 0 | 0 | 0 | 0 | 3 | 6 | 8 | 9 | 9 |
| GSK620) | 2 | 0 | 0 | 0 | 0 | 2 | 6 | 8 | 10 | 11 |
| | 3 | 0 | 0 | 0 | 0 | 0 | 0 | 8 | 6 | 6 |
| | 4 | 0 | 0 | 0 | 1 | 3 | 4 | 8 | 9 | 9 |
| | 5 | 0 | 0 | 0 | 0 | 0 | 1 | 4 | 5 | 6 |
| | 6 | 0 | 0 | 0 | 0 | 0 | 2 | 6 | 7 | 7 |
| | 7 | 0 | 0 | 0 | 0 | 4 | 6 | 8 | 8 | 8 |
| | 8 | 0 | 0 | 0 | 0 | 4 | 4 | 8 | 7 | 7 |
| 5 (CIA + | 1 | 0 | 0 | 0 | 1 | 2 | 2 | 7 | 9 | 9 |
| Example 101 | 2 | 0 | 0 | 0 | 1 | 2 | 3 | 7 | 7 | 7 |
| 3 mg/kg) | 3 | 0 | 0 | 1 | 2 | 2 | 3 | 8 | 8 | 8 |
| | 4 | 0 | 0 | 1 | 2 | 4 | 6 | 8 | 16 | 14 |
| | 5 | 0 | 0 | 0 | 0 | 1 | 3 | 8 | 7 | 7 |
| | 6 | 0 | 0 | 1 | 3 | 6 | 8 | 12 | 12 | 13 |
| | 7 | 0 | 0 | 0 | 0 | 1 | 6 | 12 | 12 | 12 |
| | 8 | 0 | 0 | 0 | 0 | 1 | 4 | 8 | 8 | 8 |
| 6 (CIA + | 1 | 0 | 0 | 1 | 3 | 4 | 5 | 7 | 7 | 7 |
| Example 101 | 2 | 0 | 0 | 0 | 0 | 4 | 4 | 6 | 6 | 6 |
| 10 mg/kg) | 3 | 0 | 0 | 2 | 4 | 6 | 7 | 12 | 12 | 12 |
| | 4 | 0 | 0 | 1 | 2 | 5 | 6 | 11 | 11 | 12 |
| | 5 | 0 | 0 | 0 | 0 | 0 | 0 | 1 | 2 | 2 |
| | 6 | 0 | 0 | 0 | 1 | 0 | 1 | 6 | 7 | 7 |
| | 7 | 0 | 0 | 0 | 0 | 0 | 1 | 2 | 3 | 4 |
| | 8 | 0 | 0 | 0 | 0 | 0 | 2 | 0 | 3 | 2 |
| 7 (CIA + | 1 | 0 | 0 | 1 | 1 | 1 | 1 | 0 | 0 | 0 |
| Example 101 | 2 | 0 | 0 | 0 | 0 | 0 | 2 | 5 | 6 | 6 |
| 30 mg/kg) | 3 | 0 | 0 | 0 | 0 | 0 | 2 | 4 | 7 | 7 |
| | 4 | 0 | 0 | 0 | 0 | 0 | 0 | 0 | 1 | 1 |
| | 5 | 0 | 0 | 0 | 0 | 0 | 0 | 0 | 1 | 1 |
| | 6 | 0 | 0 | 0 | 0 | 0 | 0 | 1 | 6 | 6 |
| | 7 | 0 | 0 | 0 | 0 | 0 | 2 | 4 | 5 | 6 |
| | 8 | 0 | 0 | 0 | 0 | 0 | 0 | 2 | 3 | 2 |

TABLE 15D
Mean overall clinical score ± SD

| Group | Day 0 | Day 6 | Day 12 | Day 13 | Day 14 | Day 15 | Day 18 | Day 19 | Day 21 |
|---|---|---|---|---|---|---|---|---|---|
| 1 (No CIA + | 0 ± 0 | 0 ± 0 | 0 ± 0 | 0 ± 0 | 0 ± 0 | 0 ± 0* | 0 ± 0** | 0 ± 0** | 0 ± 0** |
| Vehicle) | | | | | | | | | |
| 2 (CIA + | 0 ± 0 | 0 ± 0 | 1 ± 2 | 2 ± 4 | 5 ± 4 | 7 ± 4 | 12 ± 3 | 12 ± 3 | 13 ± 3 |
| Vehicle) | | | | | | | | | |
| 3 (CIA + | 0 ± 0 | 0 ± 0 | 0 ± 0 | 0 ± 0 | 0 ± 0 | 0 ± 0* | 0 ± 0** | 0 ± 0** | 0 ± 0** |
| Dex) | | | | | | | | | |
| 4 (CIA + | 0 ± 0 | 0 ± 0 | 0 ± 0 | 0 ± 0 | 2 ± 2 | 4 ± 2 | 7 ± 1* | 8 ± 2 | 8 ± 2 |
| GSK620) | | | | | | | | | |
| 5 (CIA + | 0 ± 0 | 0 ± 0 | 0 ± 1 | 1 ± 1 | 2 ± 2 | 4 ± 2 | 9 ± 2 | 10 ± 3 | 10 ± 3 |
| Example 101 | | | | | | | | | |
| 3 mg/kg) | | | | | | | | | |
| 6 (CIA + | 0 ± 0 | 0 ± 0 | 1 ± 1 | 1 ± 2 | 2 ± 3 | 3 ± 3 | 6 ± 4 | 6 ± 4 | 7 ± 4 |
| Example 101 | | | | | | | | | |
| 10 mg/kg) | | | | | | | | | |
| 7 (CIA + | 0 ± 0 | 0 ± 0 | 0 ± 0 | 0 ± 0 | 0 ± 0 | 1 ± 1 | 2 ± 2** | 4 ± 3** | 4 ± 3** |
| Example 101 | | | | | | | | | |
| 30 mg/kg) | | | | | | | | | |

One-way ANOVA comparison to Group 2;
*p < 0.05;
**p < 0.01

TABLE 15E
Clinical observations

| | | Clinical Observation | | | | | | | | | |
|---|---|---|---|---|---|---|---|---|---|---|---|
| Group | # | Day 12 | Day 13 | Day 14 | Day 15 | Day 16 | Day 17 | Day 18 | Day 19 | Day 20 | Day 21 |
| 1 (No CIA + | 1 | — | — | — | — | — | — | — | — | — | — |
| Vehicle) | 2 | — | — | — | — | — | — | — | — | — | — |
| | 3 | — | — | — | — | — | — | — | — | — | — |
| | 4 | — | — | — | — | — | — | — | — | — | — |
| | 5 | — | — | — | — | — | — | — | — | — | — |
| | 6 | — | — | — | — | — | — | — | — | — | — |
| | 7 | — | — | — | — | — | — | — | — | — | — |
| | 8 | — | — | — | — | — | — | — | — | — | — |
| 2 (CIA + | 1 | — | — | — | — | — | — | — | — | — | — |
| Vehicle) | 2 | — | — | — | — | — | — | — | — | — | — |
| | 3 | — | — | — | — | — | — | — | — | — | — |
| | 4 | — | — | — | — | — | — | — | — | — | — |
| | 5 | — | — | — | — | — | — | — | — | — | — |
| | 6 | — | — | — | — | — | — | — | — | — | — |
| | 7 | — | — | — | — | — | — | — | — | — | — |
| | 8 | — | — | — | — | — | — | — | — | — | — |
| 3 (CIA + | 1 | — | — | — | — | — | — | — | — | — | — |
| Dexamethasone) | 2 | — | — | — | — | — | — | — | — | — | — |
| | 3 | — | — | — | — | — | — | — | — | — | — |
| | 4 | — | — | — | — | — | — | — | — | — | — |
| | 5 | — | — | — | — | — | — | — | — | — | — |
| | 6 | — | — | — | — | — | — | — | — | — | — |
| | 7 | — | — | — | — | — | — | — | — | — | — |
| | 8 | — | — | — | — | — | — | — | — | — | — |
| 4 (CIA + | 1 | — | — | — | — | — | — | — | — | — | — |
| GSK620) | 2 | — | — | — | — | — | — | — | — | — | — |
| | 3 | — | — | — | — | — | — | — | — | — | — |
| | 4 | — | — | — | — | — | — | — | — | — | — |
| | 5 | — | — | — | — | — | — | — | — | — | — |
| | 6 | — | — | — | — | — | — | — | — | — | — |
| | 7 | — | — | — | — | — | — | — | — | — | — |
| | 8 | — | — | — | — | — | — | — | — | — | — |
| 5 (CIA + | 1 | — | — | — | — | — | — | — | — | — | — |
| Example 101 | 2 | — | — | — | — | — | — | — | — | — | — |
| 3 mg/kg) | 3 | — | — | — | — | — | — | — | — | — | — |
| | 4 | — | — | — | — | — | — | — | — | — | — |
| | 5 | — | — | — | — | — | — | — | — | — | — |
| | 6 | — | — | — | — | — | — | — | — | — | — |
| | 7 | — | — | — | — | — | — | — | — | — | — |
| | 8 | — | — | — | — | — | — | — | — | — | — |
| 6 (CIA + | 1 | — | — | — | Diarrhea | — | — | — | — | — | — |
| Example 101 | 2 | — | — | — | — | — | — | — | — | — | — |
| 10 mg/kg) | 3 | — | — | — | — | — | — | — | — | — | — |
| | 4 | — | — | — | — | — | — | — | — | — | — |
| | 5 | — | — | — | — | — | — | — | — | — | — |
| | 6 | — | — | — | — | — | — | — | Diarrhea | — | — |
| | 7 | — | — | — | — | — | — | — | — | — | — |
| | 8 | — | — | — | — | — | — | — | — | — | — |
| 7 (CIA + | 1 | — | Diarrhea | — | — | — | — | — | Diarrhea | — | — |
| Example 101 | 2 | — | — | — | — | — | — | — | — | — | — |
| 30 mg/kg) | 3 | Diarrhea | — | — | — | — | — | — | — | — | — |
| | 4 | — | — | — | — | — | — | — | — | — | — |
| | 5 | — | — | — | — | — | — | — | — | — | — |
| | 6 | — | — | — | — | — | — | — | — | — | — |
| | 7 | — | — | — | — | — | — | Diarrhea | — | — | — |
| | 8 | — | — | — | — | Diarrhea | — | — | Diarrhea | — | Diarrhea |

### Tables—Part B: IgG1 Measurements

TABLE 16A
Rat anti-collagen IgG1 levels

| | | | | | | | | Final |
|---|---|---|---|---|---|---|---|---|
| | | | | | | Rat Anti- | | Rat Anti- |
| | | | | | | Collagen | | Collagen |
| | | | | | | IgG1 | | IgG1 |
| | | | | | | Antibody | | Antibody |
| | | | | Average | Calculated | Con. | | Con. |
| Group | ID | OD450 nm | | OD450 nm | O.D. | (units/ml) | Fold | (units/ml) |
| 1 (No CIA + | 1 | 0.0576 | 0.0577 | 0.0577 | 0.0080 | 0.60 | 2 | 1.19 |
| Vehicle) | 2 | 0.0544 | 0.0580 | 0.0562 | 0.0066 | 0.49 | 2 | 0.98 |
| | 3 | 0.0557 | 0.0581 | 0.0569 | 0.0073 | 0.54 | 2 | 1.08 |
| | 4 | 0.0753 | 0.0707 | 0.0730 | 0.0234 | 1.79 | 2 | 3.58 |
| | 5 | 0.0649 | 0.0669 | 0.0659 | 0.0163 | 1.22 | 2 | 2.45 |
| | 6 | 0.0622 | 0.0560 | 0.0591 | 0.0095 | 0.70 | 2 | 1.41 |
| | 7 | 0.0654 | 0.0671 | 0.0663 | 0.0166 | 1.25 | 2 | 2.50 |
| | 8 | 0.0617 | 0.0618 | 0.0618 | 0.0121 | 0.90 | 2 | 1.81 |
| 2 (CIA + | 1 | 0.2248 | 0.2282 | 0.2265 | 0.1769 | 16.15 | 2000 | 32306.30 |
| Vehicle) | 2 | 0.1409 | 0.1352 | 0.1381 | 0.0884 | 7.49 | 2000 | 14970.83 |
| | 3 | 0.1044 | 0.1020 | 0.1032 | 0.0536 | 4.34 | 2000 | 8675.25 |
| | 4 | 0.0908 | 0.0893 | 0.0901 | 0.0404 | 3.20 | 2000 | 6404.64 |
| | 5 | 0.2026 | 0.1990 | 0.2008 | 0.1512 | 13.55 | 2000 | 27093.89 |
| | 6 | 0.1050 | 0.1069 | 0.1060 | 0.0563 | 4.58 | 2000 | 9158.20 |
| | 7 | 0.1645 | 0.1660 | 0.1653 | 0.1156 | 10.06 | 2000 | 20113.89 |
| | 8 | 0.1565 | 0.1590 | 0.1578 | 0.1081 | 9.34 | 2000 | 18677.72 |
| 3 (CIA + | 1 | 0.1744 | 0.1758 | 0.1751 | 0.1255 | 11.01 | 2000 | 22019.88 |
| Dexamethasone) | 2 | 0.0834 | 0.0830 | 0.0832 | 0.0336 | 2.63 | 2000 | 5250.27 |
| | 3 | 0.0587 | 0.0580 | 0.0584 | 0.0087 | 0.65 | 2000 | 1296.14 |
| | 4 | 0.0648 | 0.0682 | 0.0665 | 0.0169 | 1.27 | 2000 | 2541.14 |
| | 5 | 0.1219 | 0.1189 | 0.1204 | 0.0708 | 5.87 | 2000 | 11736.83 |
| | 6 | 0.0778 | 0.0802 | 0.0790 | 0.0294 | 2.28 | 2000 | 4553.59 |
| | 7 | 0.0997 | 0.0981 | 0.0989 | 0.0493 | 3.96 | 2000 | 7925.50 |
| | 8 | 0.0970 | 0.0941 | 0.0956 | 0.0459 | 3.67 | 2000 | 7346.16 |
| 4 (CIA + | 1 | 0.1048 | 0.1070 | 0.1059 | 0.0563 | 4.57 | 2000 | 9149.39 |
| GSK620) | 2 | 0.1158 | 0.1165 | 0.1162 | 0.0665 | 5.49 | 2000 | 10971.56 |
| | 3 | 0.0510 | 0.0510 | 0.0510 | 0.0014 | 0.13 | 2000 | 258.62 |
| | 4 | 0.0597 | 0.0584 | 0.0591 | 0.0094 | 0.70 | 2000 | 1400.18 |
| | 5 | 0.1344 | 0.1422 | 0.1383 | 0.0887 | 7.51 | 2000 | 15017.25 |
| | 6 | 0.0652 | 0.0696 | 0.0674 | 0.0178 | 1.34 | 2000 | 2682.44 |
| | 7 | 0.1051 | 0.1085 | 0.1068 | 0.0572 | 4.65 | 2000 | 9308.00 |
| | 8 | 0.1186 | 0.1193 | 0.1190 | 0.0693 | 5.74 | 2000 | 11475.12 |
| 5 (CIA + | 1 | 0.1334 | 0.1366 | 0.1350 | 0.0854 | 7.20 | 1000 | 7202.91 |
| Example 101 | 2 | 0.1187 | 0.1192 | 0.1190 | 0.0693 | 5.74 | 1000 | 5737.56 |
| 3 mg/kg) | 3 | 0.0532 | 0.0621 | 0.0577 | 0.0080 | 0.60 | 1000 | 596.39 |
| | 4 | 0.2278 | 0.2424 | 0.2351 | 0.1855 | 17.04 | 1000 | 17040.09 |
| | 5 | 0.1281 | 0.1339 | 0.1310 | 0.0814 | 6.83 | 1000 | 6834.32 |
| | 6 | 0.0789 | 0.0811 | 0.0800 | 0.0304 | 2.36 | 1000 | 2359.32 |
| | 7 | 0.0717 | 0.0689 | 0.0703 | 0.0207 | 1.57 | 1000 | 1570.97 |
| | 8 | 0.2381 | 0.2243 | 0.2312 | 0.1816 | 16.64 | 1000 | 16636.97 |
| 6 (CIA + | 1 | 0.4650 | 0.4817 | 0.4734 | 0.4237 | 44.34 | 1000 | 44339.86 |
| Example 101 | 2 | 0.1629 | 0.1576 | 0.1603 | 0.1106 | 9.58 | 1000 | 9577.48 |
| 10 mg/kg) | 3 | 0.2259 | 0.2059 | 0.2159 | 0.1663 | 15.07 | 1000 | 15070.08 |
| | 4 | 0.0591 | 0.0548 | 0.0570 | 0.0073 | 0.55 | 1000 | 545.06 |
| | 5 | 0.1978 | 0.1741 | 0.1860 | 0.1363 | 12.07 | 1000 | 12072.29 |
| | 6 | 0.0995 | 0.0939 | 0.0967 | 0.0471 | 3.77 | 1000 | 3772.28 |
| | 7 | 0.0617 | 0.0586 | 0.0602 | 0.0105 | 0.78 | 1000 | 782.47 |
| | 8 | 0.0588 | 0.0555 | 0.0572 | 0.0075 | 0.56 | 1000 | 559.69 |
| 7 (CIA + | 1 | 0.0504 | 0.0543 | 0.0524 | 0.0027 | 0.22 | 1000 | 219.33 |
| Example 101 | 2 | 0.0486 | 0.0519 | 0.0503 | 0.0006 | 0.08 | 1000 | 81.22 |
| 30 mg/kg) | 3 | 0.0474 | 0.0479 | 0.0477 | −0.0020 | ND | 1000 | ND |
| | 4 | 0.0630 | 0.0684 | 0.0657 | 0.0161 | 1.21 | 1000 | 1208.05 |
| | 5 | 0.1069 | 0.0989 | 0.1029 | 0.0533 | 4.31 | 1000 | 4311.36 |
| | 6 | 0.0770 | 0.0693 | 0.0732 | 0.0235 | 1.80 | 1000 | 1799.62 |
| | 7 | 0.0563 | 0.0577 | 0.0570 | 0.0074 | 0.55 | 1000 | 548.72 |
| | 8 | 0.0734 | 0.0769 | 0.0752 | 0.0255 | 1.96 | 1000 | 1961.63 |

TABLE 16B
Average rat anti-collagen IgG1 levels

| Group | Mean | SEM |
|---|---|---|
| 1 (No CIA + Vehicle) | 1.87** | 0.32 |
| 2 (CIA + Vehicle) | 17175.09 | 3261.27 |
| 3 (CIA + Dex) | 7833.69* | 2333.42 |
| 4 (CIA + GSK620) | 7532.82* | 1904.87 |
| 5 (CIA + Example | 7247.32* | 2263.31 |
| 101 3 mg/kg) | | |
| 6 (CIA + Example | 10839.90 | 5185.87 |
| 101 10 mg/kg) | | |
| 7 (CIA + Example | 1447.13** | 517.01 |
| 101 30 mg/kg) | | |

One-way ANOVA comparison to Group 2;
*p < 0.05;
**p < 0.01

### Tables—Part C: Pharmacokinetics of Example 101 in Lewis Rats (Oral)

TABLE 17A
Day 21 plasma concentration

| | | Plasma |
|---|---|---|
| Group | ID | Concentration (ng/mL) |
| 4 (CIA + GSK620) | 1 | 3230 |
| | 2 | 3450 |
| | 3 | 2540 |
| | 4 | 1590 |
| | 5 | 4170 |
| | 6 | 3070 |
| | 7 | 2740 |
| | 8 | 1760 |
| 5 (CIA + Example | 1 | 54.9 |
| 101 3 mg/kg) | 2 | 37.5 |
| | 3 | 59.4 |
| | 4 | 64.9 |
| | 5 | 49.5 |
| | 6 | 65.2 |
| | 7 | 71.9 |
| | 8 | 69.6 |
| 6 (CIA + Example | 1 | 270 |
| 101 10 mg/kg) | 2 | 134 |
| | 3 | 209 |
| | 4 | 281 |
| | 5 | 251 |
| | 6 | 313 |
| | 7 | 175 |
| | 8 | 28.4 |
| 7 (CIA + Example | 1 | 2290 |
| 101 30 mg/kg) | 2 | 1620 |
| | 3 | 1260 |
| | 4 | 1430 |
| | 5 | 1220 |
| | 6 | 1350 |
| | 7 | 2000 |
| | 8 | 1440 |

TABLE 17B
Day 21 average plasma concentration

| | Mean Plasma | | |
|---|---|---|---|
| Group | Concentration (ng/mL) | SD (ng/mL) | CV (%) |
| 4 (CIA + GSK620) | 2819 | 860 | 30.5 |
| 5 (CIA + Example | 59.1 | 11.5 | 19.4 |
| 101 3 mg/kg) | | | |
| 6 (CIA + Example | 208 | 93 | 44.9 |
| 101 10 mg/kg) | | | |
| 7 (CIA + Example | 1576 | 380 | 24.1 |
| 101 30 mg/kg) | | | |

TABLE 17C
Plasma concentration over time after oral
administration on day 0 (10 mg/kg)

| | Concentration (ng/mL) | | |
|---|---|---|---|
| | Test | Test | Test |
| Time (h) | Subject 1 | Subject 2 | Subject 3 |
| 0.25 | 375 | 378 | 754 |
| 0.5 | 738 | 673 | 707 |
| 1 | 1090 | 1160 | 1550 |
| 2 | 1110 | 997 | 1610 |
| 4 | 1170 | 1060 | 760 |
| 8 | 190 | 107 | 147 |
| 10 | 1880 | 2460 | 1460 |
| 12 | 772 | 968 | 656 |
| 14 | 324 | 525 | 469 |
| 24 | 14.3 | 35.6 | 122 |

TABLE 17D
Plasma concentration over time after oral
administration on day 20 (10 mg/kg)

| | Concentration (ng/mL) | | |
|---|---|---|---|
| | Test | Test | Test |
| Time (h) | Subject 1 | Subject 2 | Subject 3 |
| 0.25 | 1260 | 1400 | 1570 |
| 0.5 | 1660 | 1550 | 1730 |
| 1 | 1600 | 1730 | 1590 |
| 2 | 841 | 1260 | 1320 |
| 4 | 452 | 513 | 518 |
| 8 | 103 | 215 | 188 |
| 10 | 1170 | 1710 | 1460 |
| 12 | 258 | 461 | 498 |
| 14 | 68.5 | 159 | 185 |
| 24 | 16.7 | 20.1 | 38.1 |

TABLE 17E
Average plasma concentration over time after
oral administration on day 0 (10 mg/kg)

| | Mean | | |
|---|---|---|---|
| Time (h) | Concentration (ng/mL) | SD (ng/mL) | CV (%) |
| 0.25 | 502 | 218 | 43.4 |
| 0.5 | 706 | 33 | 4.61 |
| 1 | 1267 | 248 | 19.6 |
| 2 | 1239 | 326 | 26.3 |
| 4 | 997 | 212 | 21.3 |
| 8 | 148 | 42 | 28.0 |
| 10 | 1933 | 502 | 26.0 |
| 12 | 799 | 158 | 19.7 |
| 14 | 439 | 104 | 23.6 |
| 24 | 57 | 57 | 99.5 |

TABLE 17F
Average plasma concentration over time after
oral administration on day 20 (10 mg/kg)

| | Mean | | |
|---|---|---|---|
| Time (h) | Concentration (ng/mL) | SD (ng/mL) | CV (%) |
| 0.25 | 1410 | 155 | 11.0 |
| 0.5 | 1647 | 91 | 5.51 |
| 1 | 1640 | 78 | 4.76 |
| 2 | 1140 | 261 | 22.9 |
| 4 | 494 | 37 | 7.43 |
| 8 | 169 | 58 | 34.7 |
| 10 | 1447 | 270 | 18.7 |
| 12 | 406 | 129 | 31.9 |
| 14 | 138 | 61 | 44.5 |
| 24 | 25.0 | 11.5 | 46.1 |

TABLE 17G
Example 101 pharmacokinetic profiles on day 0 (10 mg/kg)

| PK | | Test | Test | Test |
|---|---|---|---|---|
| Parameters | Unit | Subject 1 | Subject 2 | Subject 3 |
| T_{1/2} | h | 2.13 | 2.54 | 5.01 |
| Tₘₐₓ | h | 10.0 | 10.0 | 2.00 |
| Cₘₐₓ | ng/mL | 1880 | 2460 | 1610 |
| AUCₗₐₛₜ | h*ng/mL | 14253 | 16397 | 14408 |
| AUC_{Inf} | h*ng/mL | 14296 | 16528 | 15290 |
| AUC_{_{—}}_{% Extrap}_obs | % | 0.307 | 0.788 | 5.77 |
| MRT_{Inf}_obs | h | 7.64 | 8.69 | 9.50 |
| AUCₗₐₛₜ/D | h*mg/mL | 713 | 820 | 720 |
| F | % | NA | NA | NA |

TABLE 17H
Example 101 pharmacokinetic profiles on day 20 (10 mg/kg)

| PK | | Test | Test | Test |
|---|---|---|---|---|
| Parameters | Unit | Subject 1 | Subject 2 | Subject 3 |
| T_{1/2} | h | 3.46 | 2.85 | 3.54 |
| Tₘₐₓ | h | 0.500 | 1.00 | 0.500 |
| Cₘₐₓ | ng/mL | 1660 | 1730 | 1730 |
| AUCₗₐₛₜ | h*ng/mL | 8415 | 11699 | 11548 |
| AUC_{Inf} | h*ng/mL | 8498 | 11782 | 11743 |
| AUC_{_{—}}_{% Extrap}_obs | % | 0.982 | 0.700 | 1.65 |
| MRT_{Inf}_obs | h | 6.09 | 6.80 | 7.09 |
| AUCₗₐₛₜ/D | h*mg/mL | 421 | 585 | 577 |
| F | % | NA | NA | NA |

TABLE 17I
Average Example 101 pharmacokinetic
profile on day 0 (10 mg/kg)

| PK | | | | |
|---|---|---|---|---|
| Parameters | Unit | Mean | SD | CV (%) |
| T_{1/2} | h | 3.22 | 1.56 | 48.3 |
| Tₘₐₓ | h | 7.3 | 4.6 | 63.0 |
| Cₘₐₓ | ng/mL | 1983 | 434 | 21.9 |
| AUCₗₐₛₜ | h*ng/mL | 15019 | 1196 | 7.96 |
| AUC_{Inf} | h*ng/mL | 15371 | 1118 | 7.27 |
| AUC_{_{—}}_{% Extrap}_obs | % | 2.29 | 3.02 | 132 |
| MRT_{Inf}_obs | h | 8.61 | 0.94 | 10.9 |
| AUCₗₐₛₜ/D | h*mg/mL | 751 | 60 | 7.96 |
| F | % | NA | NA | NA |

TABLE 17J
Average Example 101 pharmacokinetic
profile on day 20 (10 mg/kg)

| PK | | | | |
|---|---|---|---|---|
| Parameters | Unit | Mean | SD | CV (%) |
| T_{1/2} | h | 3.28 | 0.38 | 11.6 |
| Tₘₐₓ | h | 0.67 | 0.29 | 43.3 |
| Cₘₐₓ | ng/mL | 1707 | 40 | 2.37 |
| AUCₗₐₛₜ | h*ng/mL | 10554 | 1854 | 17.6 |
| AUC_{Inf} | h*ng/mL | 10674 | 1885 | 17.7 |
| AUC_{_{—}}_{% Extrap}_obs | % | 1.11 | 0.49 | 44.1 |
| MRT_{Inf}_obs | h | 6.66 | 0.51 | 7.68 |
| AUCₗₐₛₜ/D | h*mg/mL | 528 | 93 | 17.6 |
| F | % | NA | NA | NA |

TABLE 18
Observations in hind limbs from histopathologic analysis

| | Group 1 | Group 2 | Group 3 | Group 4 | Group 5 | Group 6 | Group 7 |
|---|---|---|---|---|---|---|---|
| CIA Induced? | — | Yes | Yes | Yes | Yes | Yes | Yes |
| Treatment | — | — | Dexamethasone | GSK620 | Example | Example | Example |
| | | | | | 101 (3 | 101 (10 | 101 (30 |
| | | | | | mg/kg) | mg/kg) | mg/kg) |
| Number of | 8 | 8 | 8 | 8 | 8 | 8 | 8 |
| Animals in | | | | | | | |
| Group | | | | | | | |
| Number of | 8 | 8 | 8 | 8 | 8 | 8 | 8 |
| Animals | | | | | | | |
| Examined | | | | | | | |
| No Visible | 8 | 0 | 8 | 0 | 0 | 0 | 1 |
| Lesions | | | | | | | |
| Mixed cell | | | | | | | |
| inflammation | | | | | | | |
| Minimal | 0 | 0 | 0 | 0 | 0 | 1 | 0 |
| Mild | 0 | 0 | 0 | 0 | 0 | 2 | 3 |
| Moderate | 0 | 0 | 0 | 8 | 7 | 3 | 4 |
| Marked | 0 | 8 | 0 | 0 | 1 | 2 | 0 |
| Granulation | | | | | | | |
| tissue | | | | | | | |
| Minimal | 0 | 0 | 0 | 0 | 0 | 0 | 2 |
| Mild | 0 | 0 | 0 | 4 | 1 | 3 | 1 |
| Moderate | 0 | 0 | 0 | 4 | 7 | 3 | 4 |
| Marked | 0 | 8 | 0 | 0 | 0 | 0 | 0 |
| Increased | | | | | | | |
| eroded | | | | | | | |
| surface, bone | | | | | | | |
| Minimal | 0 | 0 | 0 | 1 | 0 | 2 | 0 |
| Mild | 0 | 0 | 0 | 4 | 5 | 2 | 4 |
| Moderate | 0 | 2 | 0 | 3 | 3 | 3 | 0 |
| Marked | 0 | 6 | 0 | 0 | 0 | 0 | 0 |
| Increased | | | | | | | |
| bone, | | | | | | | |
| periosteum | | | | | | | |
| Minimal | 0 | 0 | 0 | 0 | 0 | 1 | 1 |
| Mild | 0 | 0 | 0 | 6 | 7 | 4 | 2 |
| Moderate | 0 | 0 | 0 | 2 | 1 | 2 | 0 |
| Marked | 0 | 8 | 0 | 0 | 0 | 0 | 0 |
| Erosion/Ulcer, | | | | | | | |
| cartilaginous | | | | | | | |
| Minimal | 0 | 0 | 0 | 0 | 0 | 1 | 1 |
| Mild | 0 | 0 | 0 | 6 | 2 | 3 | 4 |
| Moderate | 0 | 3 | 0 | 2 | 6 | 3 | 0 |
| Marked | 0 | 5 | 0 | 0 | 0 | 0 | 0 |

### Example 141: Cellular Activity in Human Peripheral Blood Mononuclear Cells

Tables 19A and 19B depict the results of a biomarker assay measuring levels of cytokines. Example 101 had a substantially lower 1050 for the inflammatory biomarkers IL-17A, IL-22, and CXCL-10. Example 101 showed selectivity to induction of CXCL10 relative to IL-17A and IL-22. Example 101 is seen to be more effective than GSK620. In addition to showing a superior activity relative to GSK620 in reducing inflammatory biomarkers IL-17A, IL-22, and CXCL-10, Example 101 is more selective than GSK620 for BDII by greater than about two orders of magnitude. Example 23 also exhibited a decreased mean 1050 for IL-17A and IL-22 relative to GSK620. Example 80 exhibited an increased mean 1050 for IL-17A, but a decreased mean 1050 for IL-22 relative to GSK620. Example 23 and 80 showed selectivity to induction of CXCL10 relative to IL-17A and IL-22.

TABLE 19A
Biomarker assay

| | IL-17A IC50 (nM) | | | IL-22 IC50 (nM) | | | CXCL10 IC50 (nM) | | |
|---|---|---|---|---|---|---|---|---|---|
| | Test | Test | Test | Test | Test | Test | Test | Test | Test |
| Compound | Subject 1 | Subject 2 | Subject 3 | Subject 1 | Subject 2 | Subject 3 | Subject 1 | Subject 2 | Subject 3 |
| GSK620 | 80.5 | 59.7 | 139.3 | 231.3 | 21.6 | 106.1 | >30000 | >30000 | >30000 |
| Example 101 | 20.5 | 5.7 | 58.5 | 14.8 | 26.9 | 21.1 | >30000 | >30000 | >30000 |
| Example 23 | 104. | 9.6 | 53.4 | 8.5 | 1.2 | 4.3 | — | >30000 | >30000 |
| Example 80 | 25.1 | — | 187.0 | — | 9.3 | 30.7 | >30000 | >30000 | >30000 |

TABLE 19B
Average biomarker assay

| | | | | | | | Biomarker | | Biomarker | |
|---|---|---|---|---|---|---|---|---|---|---|
| | | | | | | | selectivity | | selectivity | |
| | IL-17A | | IL-22 | | CXCL10 | | (IL-17 vs. | | (IL-22 vs. | |
| | IC50 (nM) | | IC50 (nM) | | IC50 (nM) | | CXCL10) | | CXCL10) | |
| Compound | Mean | SEM | Mean | SEM | Mean | SEM | Modifier | Fold | Modifier | Fold |
| GSK620 | 93.2 | 23.8 | 119.7 | 60.9 | >30000 | | > | 322 | > | 251 |
| Example 101 | 28.2 | 15.7 | 20.9 | 3.5 | >30000 | | > | 1063 | > | 1433 |
| Example 23 | 24.5 | 14.5 | 4.7 | 2.1 | >30000 | | > | 1226 | > | 6429 |
| Example 80 | 106.1 | 81.0 | 20.0 | 10.7 | >30000 | | > | 283 | > | 1500 |

### Example 142: Unilateral Urethral Obstruction in Animals Treated with Example 101 and Vehicle

The Unilateral Urethral Obstruction (UUO) model was used to assess the efficacy of oral delivery of Example 101 at a 3 mg/mL concentration in rats. Example 101 and control suspensions were prepared according to the procedures set out in Experimental Method A. Evaluation of the effect of Example 101 compared to vehicle and controls in UUO in rats was undertaken in accordance with the protocol set out in Experimental Method E. Experimental groups for the study are outlined in Table 10. Prior to being randomly placed in treatment groups, animals were weighed. Once randomly assigned to treatment groups, on Day 0, animals in groups 2-3 were subjected to UUO surgery.

Starting on Day 0, animals in group 2 were administered daily doses of the vehicle for 14 days. Starting on Day 0, animals in group 3 were administered daily doses of Example 101 (30 mg/kg) for 14 days. A design of the UUO study is depicted in FIG. 9B.

Body weight of all animals was recorded daily for the 14-day period. Blind clinical nephropathy scores were conducted for all animals in the study. Assessments were recording according to the clinical scoring system summarized in Table 11. Glomerulosclerosis, interstitial nephritis, collagen fiber deposition, and nephropathy were each assessed for all experimental rats, and a total clinical score was determined for each rat. The scoring system represented the degree or percentage of lesions in the whole kidney. Clinical score assessments were performed on Day 14.

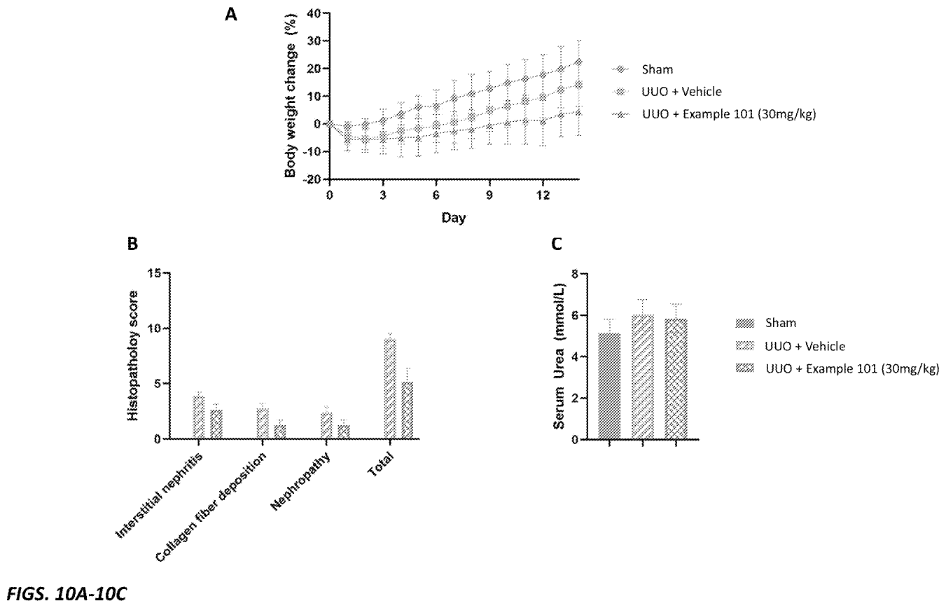

Animal body weight was measured daily over the course of the study (Tables 20A-20B; FIG. 10A). UUO animals receiving no treatment (group 2 in Table 20A-20B; square shape in FIG. 10A) exhibited a modest weight gain over the 14-day experimental period. In sham animals where UUO was not induced and no treatment was administered (group 1 in Tables 20A-20B; circle shape in FIG. 10A), the increase in weight was more pronounced relative to in UUO animals receiving no treatment. Animals receiving Example 101 (30 mg/kg; group 3 in Tables 20A-20B; triangle shape in FIG. 10A) exhibited a slight weight loss over the first seven days of the study, and then gained weight over the remaining seven days of the 14-day study, ending at an increased weight relative to starting weights.

Table 21′ shows clinical observations assessed daily in rats from each experimental group. As can be seen, two sham rats exhibited soft stools on one study day. Four UUO rats treated with Example 101 exhibited soft stools during the study. One animal exhibited soft stools on just one day, two animals exhibited soft stools on successive days, and one animal exhibited soft stools on two non-successive days. One animal in the Example 101 treatment group additionally exhibited diarrhea in addition to soft stools on successive days. Three animals in the Example 101 treatment group also exhibited dirty anuses.

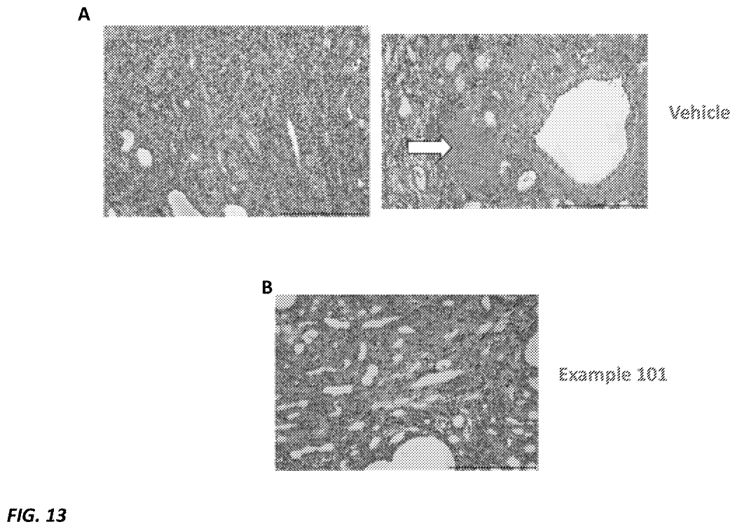

Table 22 and FIG. 10B depicts clinical histopathology scores for animals in the different treatment groups. As can be seen, treatment of UUO rats with Example 101 reduced levels of interstitial nephritis, collagen fiber deposition, and nephropathy, as well as total clinical scores relative to UUO animals receiving vehicle treatment. Representative samples of pathology staining are shown in FIG. 13A (UUO rat with vehicle treatment) and 13B (UUO rat with Example 101 treatment). UUO rats treated with vehicle (FIG. 13A) generally exhibit severe interstitial nephritis with extensive neutrophil and lymphocyte infiltration, degeneration, and necrosis in tubular epithelial cells, as well tubular dilatation. In FIG. 13A, the white arrow points to a large number of fibroblasts proliferating between renal tubules, with extensive collagen fiber deposition. UUO rats treated with Example 101 (FIG. 13B) generally exhibit mild interstitial nephritis with neutrophil and lymphocyte infiltration, degeneration and necrosis of renal tubular epithelial cells, and high tubular dilatation.

Tables 23A-23B and FIG. 10C depict serum urea levels in sham rats (solid bar in FIG. 10C), UUO rats treated with vehicle (diagonally striped bar in FIG. 10C), and UUO rats treated with Example 101 (checkered bar in FIG. 10C). No significant differences in serum urea levels between the three experimental groups were observed.

Table 24 depicts the pharmacokinetic profile of Example 101 after oral administration in Lewis rats. Blood was sampled four hours after final treatment with Example 101, and the concentration of Example 101 was measured. The mean plasma concentration of Example 101 in rats was 846 ng/mL. The pharmacokinetic profile indicates that Example 101 may be therapeutically effective with a single daily unit dose.

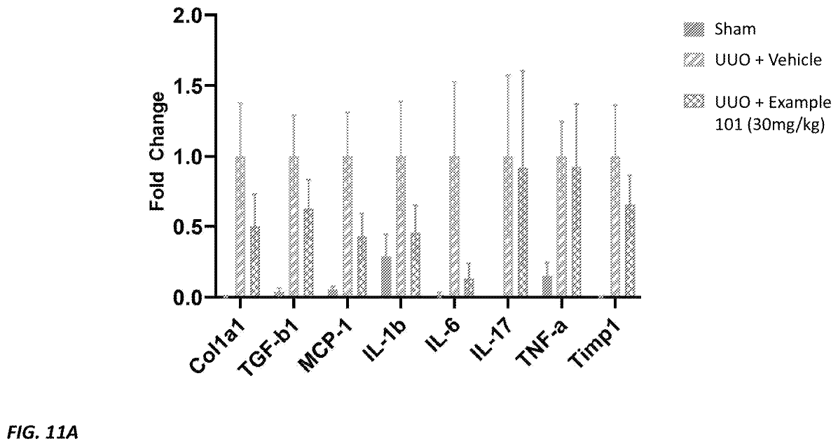

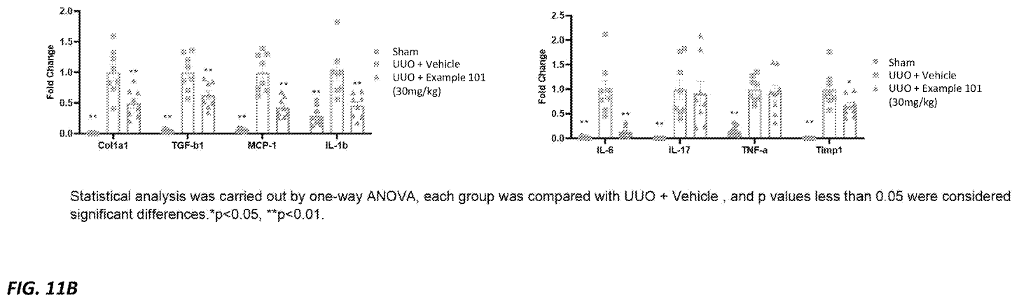

Tables 25A-25B and FIGS. 11A-11B show tissue RNA levels of various biomarkers (Col1a1, TGB-b1, MCP-1, IL-1b, IL-6, IL-17, TNF-α, and Timp1) in sham rats (solid bar in FIG. 11A; circle shape in FIG. 11B), UUO rats treated with vehicle (diagonally striped bar in FIG. 11A; square shape in FIG. 11B), and UUO rats treated with Example 101 (checkered bar in FIG. 11A; triangle shape in FIG. 11B). Treatment of UUO rats with Example 101 reduced levels of Col1a1, TGB-b1, MCP-1, IL-1b, IL-6, and Timp1 relative to animals in the UUO+vehicle treatment group. Example 101 exhibited a limited effect on expression of IL-17 and TNF-a relative to animals in the UUO+vehicle group.

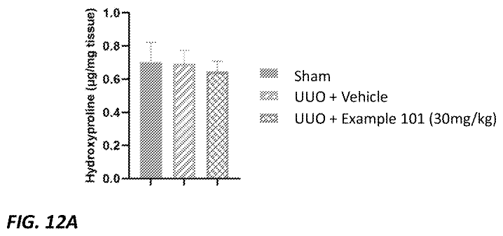

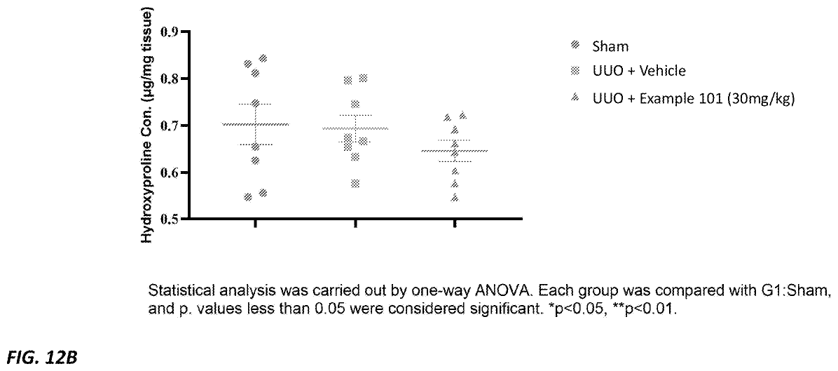

Table 26 and FIGS. 12A-B depict hydroxyproline levels in sham rats (solid bar in FIG. 12A; circle shape in FIG. 12B), UUO rats treated with vehicle (diagonally striped bar in FIG. 12A; square shape in FIG. 12B), and UUO rats treated with Example 101 (checkered bar in FIG. 12A; triangle shape in FIG. 12B). No significant differences in hydroxyproline levels between the three experimental groups were observed.

Based on these data, 30 mg/kg of Example 101 ameliorates clinical symptoms associated with UUO.

TABLE 20A
Body Weight of Rats in UUO Study

| | | | Body weight (g) | | | | | | | | |
|---|---|---|---|---|---|---|---|---|---|---|---|
| Group | Treatment | ID | Day −1 | Day 0 | Day 1 | Day 2 | Day 3 | Day 4 | Day 5 | Day 6 | Day 7 |
| 1 | Sham | 1 | 302 | 309 | 312 | 315 | 324 | 333 | 334 | 337 | 345 |
| | | 2 | 285 | 291 | 287 | 289 | 291 | 301 | 308 | 310 | 318 |
| | | 3 | 291 | 299 | 294 | 294 | 278 | 284 | 290 | 295 | 301 |
| | | 4 | 285 | 301 | 297 | 301 | 309 | 319 | 332 | 339 | 352 |
| | | 5 | 298 | 315 | 302 | 307 | 315 | 325 | 334 | 305 | 313 |
| | | 6 | 291 | 295 | 295 | 297 | 305 | 317 | 322 | 325 | 335 |
| | | 7 | 302 | 309 | 308 | 314 | 328 | 324 | 333 | 343 | 352 |
| | | 8 | 297 | 302 | 302 | 305 | 302 | 309 | 318 | 321 | 326 |
| 2 | UUO + | 1 | 299 | 310 | 293 | 290 | 294 | 297 | 305 | 310 | 296 |
| | Vehicle | 2 | 288 | 289 | 296 | 291 | 293 | 302 | 306 | 316 | 325 |
| | | 3 | 283 | 284 | 269 | 268 | 263 | 263 | 266 | 257 | 251 |
| | | 4 | 294 | 304 | 293 | 289 | 297 | 300 | 308 | 307 | 317 |
| | | 5 | 301 | 308 | 279 | 274 | 272 | 281 | 280 | 287 | 292 |
| | | 6 | 297 | 292 | 280 | 276 | 284 | 293 | 297 | 297 | 304 |
| | | 7 | 304 | 317 | 295 | 285 | 294 | 294 | 305 | 307 | 321 |
| | | 8 | 285 | 290 | 285 | 284 | 289 | 296 | 286 | 297 | 307 |
| 3 | UUO + | 1 | 306 | 324 | 293 | 299 | 306 | 315 | 310 | 323 | 322 |
| | Example 101 | 2 | 287 | 307 | 284 | 274 | 266 | 259 | 263 | 265 | 270 |
| | (30 mg/kg) | 3 | 299 | 295 | 277 | 274 | 267 | 262 | 254 | 258 | 258 |
| | | 4 | 295 | 302 | 279 | 274 | 274 | 276 | 282 | 286 | 297 |
| | | 5 | 281 | 285 | 276 | 275 | 278 | 283 | 284 | 285 | 289 |
| | | 6 | 285 | 297 | 277 | 273 | 274 | 277 | 279 | 284 | 287 |
| | | 7 | 296 | 294 | 302 | 303 | 304 | 309 | 302 | 304 | 307 |
| | | 8 | 301 | 306 | 284 | 299 | 305 | 312 | 315 | 321 | 324 |

| | | | Body weight (g) | | | | | | |
|---|---|---|---|---|---|---|---|---|---|
| Group | Treatment | ID | Day 8 | Day 9 | Day 10 | Day 11 | Day 12 | Day 13 | Day 14 |
| 1 | Sham | 1 | 347 | 350 | 360 | 363 | 363 | 374 | 379 |
| | | 2 | 321 | 325 | 332 | 334 | 342 | 343 | 352 |
| | | 3 | 302 | 311 | 315 | 318 | 319 | 324 | 331 |
| | | 4 | 362 | 374 | 378 | 386 | 393 | 400 | 410 |
| | | 5 | 320 | 335 | 339 | 343 | 353 | 356 | 363 |
| | | 6 | 336 | 340 | 343 | 350 | 355 | 369 | 374 |
| | | 7 | 369 | 362 | 370 | 377 | 380 | 388 | 395 |
| | | 8 | 329 | 334 | 341 | 345 | 344 | 348 | 355 |
| 2 | UUO + | 1 | 310 | 324 | 324 | 330 | 336 | 346 | 347 |
| | Vehicle | 2 | 332 | 338 | 342 | 354 | 355 | 359 | 370 |
| | | 3 | 258 | 264 | 272 | 272 | 285 | 295 | 296 |
| | | 4 | 322 | 327 | 340 | 339 | 336 | 347 | 351 |
| | | 5 | 290 | 295 | 295 | 299 | 307 | 316 | 321 |
| | | 6 | 312 | 315 | 316 | 326 | 320 | 330 | 332 |
| | | 7 | 327 | 336 | 347 | 351 | 360 | 364 | 377 |
| | | 8 | 297 | 305 | 309 | 317 | 322 | 330 | 341 |
| 3 | UUO + | 1 | 322 | 331 | 330 | 327 | 318 | 328 | 334 |
| | Example 101 | 2 | 271 | 276 | 277 | 275 | 285 | 283 | 286 |
| | (30 mg/kg) | 3 | 260 | 264 | 261 | 265 | 260 | 276 | 271 |
| | | 4 | 296 | 303 | 311 | 297 | 291 | 304 | 313 |
| | | 5 | 292 | 296 | 301 | 305 | 310 | 313 | 312 |
| | | 6 | 290 | 291 | 295 | 300 | 300 | 308 | 312 |
| | | 7 | 309 | 309 | 320 | 330 | 331 | 334 | 339 |
| | | 8 | 326 | 329 | 330 | 345 | 340 | 347 | 348 |

TABLE 20B
Mean ± SD of Body Weight of Rats in UUO Study

| | | Body weight (g) | | | | | | | | |
|---|---|---|---|---|---|---|---|---|---|---|
| Group | Treatment | Day −1 | Day 0 | Day 1 | Day 2 | Day 3 | Day 4 | Day 5 | Day 6 | Day 7 |
| 1 | Sham | 294 ± | 303 ± | 300 ± | 303 ± | 307 ± | 314 ± | 321 ± | 322 ± | 330 ± |
| | | 7 | 8 | 8 | 9 | 17 | 16 | 16 | 17 | 19 |
| 2 | UUO + | 294 ± | 299 ± | 286 ± | 282 ± | 286 ± | 291 ± | 294 ± | 297 ± | 302 ± |
| | Vehicle | 8 | 12 | 10 | 8 | 12 | 13 | 15 | 19 | 24 |
| 3 | UUO + | 294 ± | 301 ± | 284 ± | 284 ± | 284 ± | 287 ± | 286 ± | 291 ± | 294 ± |
| | Example 101 | 9 | 12 | 9 | 14 | 18 | 22 | 22 | 24 | 23 |

| | | Body weight (g) | | | | | | |
|---|---|---|---|---|---|---|---|---|
| Group | Treatment | Day 8 | Day 9 | Day 10 | Day 11 | Day 12 | Day 13 | Day 14 |
| 1 | Sham | 336 ± | 341 ± | 347 ± | 352 ± | 356 ± | 363 ± | 370 ± |
| | | 23 | 20 | 21 | 22 | 23 | 25 | 25 |
| 2 | UUO + | 306 ± | 313 ± | 318 ± | 324 ± | 328 ± | 336 ± | 342 ± |
| | Vehicle | 24 | 25 | 26 | 27 | 25 | 23 | 26 |
| 3 | UUO + | 296 ± | 300 ± | 303 ± | 306 ± | 304 ± | 312 ± | 314 ± |
| | Example 101 | 23 | 23 | 25 | 28 | 26 | 24 | 26 |

TABLE 21
Clinical Observation/Assessment of Rats in UUO Study

| | | | Clinical Observation | | | | | | | |
|---|---|---|---|---|---|---|---|---|---|---|
| Group | Treatment | No. | Day 0 | Day 1 | Day 2 | Day 3 | Day 4 | Day 5 | Day 6 | Day 7 |
| 1 | Sham | 1 | — | — | — | — | — | — | — | — |
| | | 2 | — | — | — | — | — | — | — | — |
| | | 3 | — | — | — | — | — | — | — | — |
| | | 4 | — | — | — | — | — | soft | — | — |
| | | | | | | | | stool | | |
| | | 5 | — | — | — | — | — | — | — | — |
| | | 6 | — | — | — | — | — | — | — | — |
| | | 7 | — | — | — | — | — | soft | — | — |
| | | | | | | | | stool | | |
| | | 8 | — | — | — | — | — | — | — | — |
| 2 | UUO + | 1 | — | — | — | — | — | — | — | — |
| | Vehicle | 2 | — | — | — | — | — | — | — | — |
| | | 3 | — | — | — | — | — | — | — | — |
| | | 4 | — | — | — | — | — | — | — | — |
| | | 5 | — | — | — | — | — | — | — | — |
| | | 6 | — | — | — | — | — | — | — | — |
| | | 7 | — | — | — | — | — | — | — | — |
| | | 8 | — | — | — | — | — | — | — | — |
| 3 | UUO + | 1 | — | — | — | — | — | Anal | Anal | Anal |
| | Example 101 | | | | | | | dirty | dirty | dirty |
| | | 2 | — | — | — | — | — | — | — | — |
| | | 3 | — | — | — | — | — | — | — | — |
| | | 4 | — | — | — | — | — | — | — | — |
| | | 5 | — | — | — | diarrhea | — | — | — | — |
| | | 6 | — | — | — | — | — | — | — | — |
| | | 7 | — | — | — | — | — | — | — | — |
| | | 8 | — | — | — | — | — | — | — | — |

| | | | Clinical Observation | | | | | | |
|---|---|---|---|---|---|---|---|---|---|
| Group | Treatment | No. | Day 8 | Day 9 | Day 10 | Day 11 | Day 12 | Day 13 | Day 14 |
| 1 | Sham | 1 | — | — | — | — | — | — | — |
| | | 2 | — | — | — | — | — | — | — |
| | | 3 | — | — | — | — | — | — | — |
| | | 4 | — | — | — | — | — | — | — |
| | | 5 | — | — | — | — | — | — | — |
| | | 6 | — | — | — | — | — | — | — |
| | | 7 | — | — | — | — | — | — | — |
| | | 8 | — | — | — | — | — | — | — |
| 2 | UUO + | 1 | — | — | — | — | — | — | — |
| | Vehicle | 2 | — | — | — | — | — | — | — |
| | | 3 | — | — | — | — | — | — | — |
| | | 4 | — | — | — | — | — | — | — |
| | | 5 | — | — | — | — | — | — | — |
| | | 6 | — | — | — | — | — | — | — |
| | | 7 | — | — | — | — | — | — | — |
| | | 8 | — | — | — | — | — | — | — |
| 3 | UUO + | 1 | soft | — | — | — | — | soft | — |
| | Example 101 | | stool | | | | | stool | |
| | | 2 | — | — | — | — | — | soft | Anal |
| | | | | | | | | stool | dirty |
| | | 3 | — | — | — | — | — | — | Anal |
| | | | | | | | | | dirty |
| | | 4 | — | — | — | — | — | — | — |
| | | 5 | — | soft | soft | — | — | — | — |
| | | | | stool | stool | | | | |
| | | 6 | — | — | — | — | — | soft | soft |
| | | | | | | | | stool | stool |
| | | 7 | — | — | — | — | — | — | — |
| | | 8 | — | — | — | — | — | — | — |

TABLE 22
Clinical Scores of Glomerulosclerosis, Interstitial Nephritis, Collagen Fiber
Deposition, Nephropathy, as well as Total Scores in Rats in UUO Study

| | | | | Collagen | | |
|---|---|---|---|---|---|---|
| | Animal | | Interstitial | fiber | | Total |
| Group | No. | Glomerulosclerosis | nephritis | deposition | Nephropathy | score |
| Sham | 1 | 0 | 0 | 0 | 0 | 0 |
| | 2 | 0 | 0 | 0 | 0 | 0 |
| | 3 | 0 | 0 | 0 | 0 | 0 |
| | 4 | 0 | 0 | 0 | 0 | 0 |
| | 5 | 0 | 0 | 0 | 0 | 0 |
| | 6 | 0 | 0 | 0 | 0 | 0 |
| | 7 | 0 | 0 | 0 | 0 | 0 |
| | 8 | 0 | 0 | 0 | 0 | 0 |
| UUO + | 1 | 0 | 4 | 3 | 2 | 9 |
| Vehicle | 2 | 0 | 4 | 2 | 3 | 9 |
| | 3 | 0 | 4 | 3 | 2 | 9 |
| | 4 | 0 | 4 | 3 | 2 | 9 |
| | 5 | 0 | 4 | 2 | 3 | 9 |
| | 6 | 0 | 4 | 3 | 2 | 9 |
| | 7 | 0 | 3 | 3 | 2 | 8 |
| | 8 | 0 | 4 | 3 | 3 | 10 |
| UUO + | 1 | 0 | 3 | 2 | 2 | 7 |
| Example 101 | 2 | 0 | 3 | 1 | 1 | 5 |
| | 3 | 0 | 2 | 1 | 1 | 4 |
| | 4 | 0 | 3 | 2 | 2 | 7 |
| | 5 | 0 | 3 | 1 | 1 | 5 |
| | 6 | 0 | 2 | 1 | 1 | 4 |
| | 7 | 0 | 2 | 1 | 1 | 4 |
| | 8 | 0 | 3 | 1 | 1 | 5 |

TABLE 23A
Serum Urea Levels of Rats in UUO Study

| | | | Serum Urea |
|---|---|---|---|
| Group | Treatment | ID | (mmol/L) |
| 1 | Sham | 1 | 4.12 |
| | | 2 | 4.57 |
| | | 3 | 5.38 |
| | | 4 | 4.68 |
| | | 5 | 4.96 |
| | | 6 | 5.76 |
| | | 7 | 5.30 |
| | | 8 | 6.23 |
| 2 | UUO + Vehicle | 1 | 6.27 |
| | | 2 | 4.62 |
| | | 3 | 7.03 |
| | | 4 | 5.44 |
| | | 5 | 6.21 |
| | | 6 | 6.63 |
| | | 7 | 5.93 |
| | | 8 | 6.14 |
| 3 | UUO + Example | 1 | 4.95 |
| | 101 (30 mg/kg) | 2 | 6.04 |
| | | 3 | 6.62 |
| | | 4 | 5.01 |
| | | 5 | 5.28 |
| | | 6 | 6.26 |
| | | 7 | 5.90 |
| | | 8 | 6.70 |

TABLE 23B
Mean ± SD Serum Urea Levels of Rats in UUO Study

| | | Serum Urea |
|---|---|---|
| Group | Treatment | Mean ± SD (mmol/L) |
| 1 | Sham | 5.13 ± 0.68 |
| 2 | UUO + Vehicle | 6.03 ± 0.74 |
| 3 | UUO + Example 101 (30 mg/kg) | 5.85 ± 0.69 |

TABLE 24
Example 101 Pharmacokinetic Profile in Male SD Rats

| | Concentration (ng/ml) | | | | | | | | | | |
|---|---|---|---|---|---|---|---|---|---|---|---|
| Time | Test | Test | Test | Test | Test | Test | Test | Test | Mean | SD | CV |
| (h) | Subject 1 | Subject 2 | Subject 3 | Subject 4 | Subject 5 | Subject 6 | Subject 7 | Subject 8 | (ng/mL) | (ng/mL) | (%) |
| 4 | 485 | 959 | 760 | 859 | 906 | 1160 | 665 | 941 | 846 | 208 | 24.6 |

TABLE 25A
RNA levels of Tissue Biomarkers in Rats of UUO Study

| | | | Normalized Fold Change | | | | | | | |
|---|---|---|---|---|---|---|---|---|---|---|
| Group | Treatment | ID | Col1a1 | TGF-b1 | MCP-1 | IL-1b | IL-6 | IL-17 | TNF-a | Timp1 |
| 1 | Sham | 1 | 0.01 | 0.07 | 0.09 | 0.55 | 0.05 | ND | 0.30 | 0.00 |
| | | 2 | 0.01 | 0.04 | 0.04 | 0.25 | ND | ND | 0.19 | 0.00 |
| | | 3 | 0.00 | 0.02 | 0.03 | 0.22 | ND | ND | 0.07 | 0.00 |
| | | 4 | 0.01 | 0.08 | 0.08 | 0.47 | 0.03 | ND | 0.27 | 0.01 |
| | | 5 | 0.00 | 0.03 | 0.05 | 0.36 | 0.02 | ND | 0.10 | 0.00 |
| | | 6 | 0.00 | 0.03 | 0.05 | 0.18 | ND | ND | 0.14 | 0.00 |
| | | 7 | 0.00 | 0.02 | 0.05 | 0.14 | ND | ND | 0.07 | 0.00 |
| | | 8 | 0.00 | 0.01 | 0.03 | 0.12 | 0.01 | ND | 0.05 | 0.00 |
| 2 | UUO + | 1 | 0.82 | 0.89 | 0.82 | 0.97 | 0.81 | 0.96 | 0.87 | 0.86 |
| | Vehicle | 2 | 0.85 | 1.33 | 1.15 | 1.00 | 0.58 | 1.82 | 1.27 | 1.10 |
| | | 3 | 1.06 | 0.86 | 1.39 | 1.01 | 1.33 | 1.77 | 1.10 | 0.77 |
| | | 4 | 1.59 | 1.20 | 1.30 | 1.82 | 2.12 | 1.34 | 1.35 | 1.76 |
| | | 5 | 0.41 | 0.57 | 0.70 | 0.56 | 0.45 | 0.60 | 0.67 | 0.58 |
| | | 6 | 1.22 | 1.09 | 0.73 | 0.71 | 0.97 | 0.58 | 0.78 | 0.92 |
| | | 7 | 1.32 | 1.36 | 1.28 | 1.19 | 0.96 | 0.53 | 1.14 | 1.21 |
| | | 8 | 0.73 | 0.70 | 0.62 | 0.74 | 0.78 | 0.39 | 0.81 | 0.81 |
| 3 | UUO + | 1 | 0.67 | 0.63 | 0.34 | 0.43 | 0.06 | 0.79 | 0.90 | 0.65 |
| | Example 101 | 2 | 0.33 | 0.44 | 0.26 | 0.18 | 0.05 | 0.22 | 0.48 | 0.41 |
| | (30 mg/kg) | 3 | 0.21 | 0.35 | 0.32 | 0.29 | 0.03 | 0.24 | 0.32 | 0.44 |
| | | 4 | 0.55 | 0.81 | 0.53 | 0.69 | 0.33 | 2.09 | 1.53 | 0.71 |
| | | 5 | 0.28 | 0.52 | 0.42 | 0.55 | 0.12 | 1.81 | 1.00 | 0.54 |
| | | 6 | 0.41 | 0.50 | 0.27 | 0.27 | 0.12 | 0.54 | 0.69 | 0.56 |
| | | 7 | 0.86 | 0.90 | 0.68 | 0.56 | 0.26 | 0.70 | 0.94 | 0.94 |
| | | 8 | 0.69 | 0.85 | 0.61 | 0.67 | 0.07 | 0.94 | 1.55 | 0.97 |

TABLE 25B
Mean ± SEM RNA Levels of Tissue Biomarkers in Rats of UUO Study

| | | | Normalized Fold Change (Mean ± SEM) | | | | | | | |
|---|---|---|---|---|---|---|---|---|---|---|
| Group | Treatment | N | Col1a1 | TGF-b1 | MCP-1 | IL-1b | IL-6 | IL-17 | TNF-a | Timp1 |
| 1 | Sham | 8 | 0.01 ± | 0.04 ± | 0.05 ± | 0.29 ± | 0.03 ± | ND | 0.15 ± | 0.00 ± |
| | | | 0.00 | 0.01 | 0.01 | 0.06 | 0.01 | | 0.03 | 0.00 |
| 2 | UUO + | 8 | 1.00 ± | 1.00 ± | 1.00 ± | 1.00 ± | 1.00 ± | 1.00 ± | 1.00 ± | 1.00 ± |
| | Vehicle | | 0.13 | 0.10 | 0.11 | 0.14 | 0.19 | 0.20 | 0.09 | 0.13 |
| 3 | UUO + | 8 | 0.50 ± | 0.63 ± | 0.43 ± | 0.46 ± | 0.13 ± | 0.92 ± | 0.93 ± | 0.65 ± |
| | Example | | 0.08 | 0.07 | 0.06 | 0.07 | 0.04 | 0.24 | 0.16 | 0.07 |
| | 101 | | | | | | | | | |

TABLE 26
Hydroxyproline concentration of Rats in UUO Study

| | | | Final Hydroxyproline | | | Mean ± |
|---|---|---|---|---|---|---|
| Group | Treatment | ID | Con. (μg/mg tissue) | Mean | SEM | SEM |
| 1 | Sham | 1 | 0.56 | 0.70 | 0.04 | 0.70 ± |
| | | 2 | 0.84 | | | 0.04 |
| | | 3 | 0.65 | | | |
| | | 4 | 0.55 | | | |
| | | 5 | 0.83 | | | |
| | | 6 | 0.75 | | | |
| | | 7 | 0.63 | | | |
| | | 8 | 0.81 | | | |
| 2 | UUO + | 1 | 0.58 | 0.69 | 0.03 | 0.69 ± |
| | Vehicle | 2 | 0.80 | | | 0.03 |
| | | 3 | 0.65 | | | |
| | | 4 | 0.63 | | | |
| | | 5 | 0.80 | | | |
| | | 6 | 0.67 | | | |
| | | 7 | 0.75 | | | |
| | | 8 | 0.67 | | | |
| 3 | UUO + | 1 | 0.72 | 0.65 | 0.02 | 0.65 ± |
| | Example | 2 | 0.69 | | | 0.02 |
| | 101 | 3 | 0.64 | | | |
| | | 4 | 0.60 | | | |
| | | 5 | 0.72 | | | |
| | | 6 | 0.66 | | | |
| | | 7 | 0.58 | | | |
| | | 8 | 0.55 | | | |

1. A compound of formula (I), or a pharmaceutically acceptable salt or N-oxide thereof: 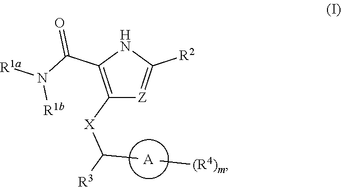
wherein:
Ring A is independently selected from phenyl, 5-membered heterocyclyl, 6-membered heterocyclyl, 9-membered bicyclic heterocyclyl, and 10-membered bicyclic heterocyclyl;
X is independently selected from O and NR⁹;
Z is independently selected from N and CR¹⁰;
R^{1a} and R^{1b} are each independently selected from H, C₁-C₄-alkyl, C₂-C₄-alkenyl, C₂-C₄-alkynyl, C₁-C₄-haloalkyl, C₀-C₄-alkylene-R^{1c}, wherein R^{1c} is independently selected from C₃-C₆ cycloalkyl, 4-membered heterocyclyl, 5-membered heterocyclyl, and 6-membered heterocyclyl; wherein R^{1c} is optionally substituted with from 1 to 4 R^{1d};
or R^{1a} and R^{1b} together with the nitrogen atom to which they are attached form a 5- to 8-membered heterocycloalkyl group optionally substituted with from 1 to 4 R^{1e};
R² is independently selected from —CONR^{2a}R^{2b}, —NR^{2a}COR^{2g}, 5-membered heterocyclyl, 6-membered heterocyclyl, and phenyl, wherein the 5-membered heterocyclyl, and 6-membered heterocyclyl groups may be optionally substituted with from 1 to 4 R^{2c} and wherein the phenyl group may be optionally substituted with from 1 to 5 R^{2c};
wherein R^{2a} and R^{2b} are each independently selected from H, C₁-C₄-alkyl, C₂-C₄-alkenyl, C₂-C₄-alkynyl, C₁-C₄-haloalkyl, C₀-C₄-alkylene-R^{2d}; wherein R^{2d} is independently selected from C₃-C₆cycloalkyl, 4-membered heterocyclyl, 5-membered heterocyclyl, and 6-membered heterocyclyl, wherein R^{2d} is optionally substituted with from 1 to 4 R^{2e};
or R^{2a} and R^{2b} together with the nitrogen atom to which they are attached form a 5- to 8-membered heterocycloalkyl group optionally substituted with from 1 to 4 R^{2f};
wherein R^{2g} is independently selected from C₁-C₄-alkyl, C₂-C₄-alkenyl, C₂-C₄-alkynyl, C₁-C₄-haloalkyl, C₀-C₄-alkylene-R^{2d}; wherein R^{2d} is independently selected from C₃-C₆ cycloalkyl, 4-membered heterocyclyl, 5-membered heterocyclyl, and 6-membered heterocyclyl, wherein R^{2d} is optionally substituted with from 1 to 4 R^{2e};
or R^{2a} and R^{2g} together with the atoms to which they are attached form a 5- to 8-membered heterocycloalkyl group optionally substituted with from 1 to 4 R¹;
R^{1d}, R^{1e}, R^{2c}, R^{2e} and R^{2f} are each independently at each occurrence selected from ═O, ═S, halo, nitro, cyano, NR⁵R⁶, OR⁷, SR⁶, SOR⁶, S(O)₂R⁶, SO₂NR⁶R⁶, CO₂R, C(O)R⁶, CONR⁶R⁶, C₁-C₄-alkyl, C₂-C₄-alkenyl, C₂-C₄-alkynyl, C₁-C₄-haloalkyl, C₃-C₄-cycloalkyl, 4-membered heterocyclyl, 5-membered heterocyclyl, and 6-membered heterocycloalkyl;
R³ is independently selected from H, C₁-C₄-alkyl, C₂-C₄-alkenyl, C₂-C₄-alkynyl, C₁-C₄-haloalkyl, C₂-C₄-haloalkenyl, C₁-C₄-alkylene-OR⁷, C₀-C₄-alkylene-S(O)₂R⁶, C₀-C₄-alkylene-CONR⁶R⁶, C₃-C₄-cycloalkyl, 4-membered heterocyclyl, 5-membered heterocyclyl, and 6-membered heterocyclyl;
R⁴ is independently at each occurrence selected from ═O, ═S, halo, nitro, cyano, C₀-C₄-alkylene-NR⁵R⁶, C₀-C₄-alkylene-OR⁷, SR⁶, SOR⁶, C₀-C₄-alkylene-S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl, C₁-C₄-alkyl-S(O)₂R⁶, C₂-C₄-alkenyl, C₂-C₄-alkynyl, C₁-C₄-haloalkyl, C₃-C₄-cycloalkyl and 4-membered heterocycloalkyl;
R⁵ is independently at each occurrence selected from H, C₁-C₄-alkyl, C(O)—C₁-C₄-alkyl and S(O)₂—C₁-C₄-alkyl; and
R⁶ is independently at each occurrence selected from H and C₁-C₄-alkyl; or where two R⁶ groups are attached to the same nitrogen, those two R⁶ groups together with the nitrogen atom to which they are attached optionally form a 5- to 8-membered-heterocycloalkyl group optionally substituted with from 1 to 4 R¹;
or R⁵ and R⁶ together with the nitrogen atom to which they are attached form a 5- to 8-membered heterocycloalkyl group optionally substituted with from 1 to 4 R¹;
R⁷ is independently at each occurrence selected from H, C₁-C₄-alkyl, C(O)—C₁-C₄-alkyl and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₄-cycloalkyl and 4-membered heterocycloalkyl;
R⁹ is independently selected from H, C₁-C₄-alkyl, C₂-C₄-alkenyl, C₂-C₄-alkynyl, C₁-C₄-haloalkyl, C₂-C₄-haloalkenyl and C₃-C₄-cycloalkyl;
R¹⁰ is independently selected from H, halo, C₁-C₄-alkyl, C₂-C₄-alkenyl, C₂-C₄-alkynyl, C₁-C₄-haloalkyl, C₂-C₄-haloalkenyl, C₀-C₄-alkylene-OR⁷ and C₃-C₆-cycloalkyl; and
m is an integer selected from 0, 1, 2, 3 and 4;
wherein any of the aforementioned alkyl, alkylene, alkenyl, or cyclopropyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a}, and S(O)₂R^{a};
wherein R^{a} is independently at each occurrence selected from H and C₁-C₄-alkyl; and R^{b} is independently at each occurrence selected from H, C₁-C₄-alkyl, C(O)—C₁-C₄-alkyl and S(O)₂—C₁-C₄-alkyl.

2. The compound of claim 1, or a pharmaceutically acceptable salt or N-oxide thereof, having a structure according to Formula (IIA):

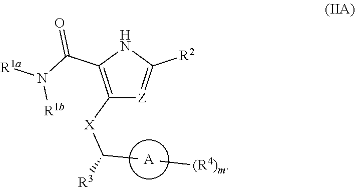

3. The compound of claim 1 or claim 2, or a pharmaceutically acceptable salt or N-oxide thereof, wherein Ring A is 5-membered heteroaryl.

4. The compound of claim 1 or claim 2, or a pharmaceutically acceptable salt or N-oxide thereof, wherein when Ring A is phenyl.

5. The compound of any one of claims 1 to 4, or a pharmaceutically acceptable salt or N-oxide thereof, wherein Z is CR¹⁰.

6. The compound of any one of claims 1 to 4, or a pharmaceutically acceptable salt or N-oxide thereof, wherein Z is N.

7. The compound of any one of claims 1 to 6, or a pharmaceutically acceptable salt or N-oxide thereof, wherein X is 0.

8. The compound of any one of claims 1 to 7, or a pharmaceutically acceptable salt or N-oxide thereof, wherein R^{1a} is C₁-C₄-alkyl and R^{1b} is H.

9. The compound of any one of claims 1 to 8, or a pharmaceutically acceptable salt or N-oxide thereof, wherein R² is —CONR^{2a}R^{2b}.

10. The compound of claim 9, or a pharmaceutically acceptable salt or N-oxide thereof, wherein R^{2a} is C₁-C₄-alkyl and R^{2b} is H.

11. The compound of any of claims 1 to 10, or a pharmaceutically acceptable salt or N-oxide thereof, wherein R³ is C₁-C₄-alkyl.

12. The compound of any one of claims 1 to 11, or a pharmaceutically acceptable salt or N-oxide thereof, wherein R⁴ is independently selected at each occurrence from C₁-C₄-alkyl, halo, cyano, C₁-C₄-haloalkyl, and C₀-C₄-alkylene-OR⁷.

13. The compound of any one of claims 1 to 12, or a pharmaceutically acceptable salt or N-oxide thereof, wherein m is an integer selected from 0 or 1.

14. The compound of claim 1, or a pharmaceutically acceptable salt or N-oxide thereof, wherein the compound according to formula (I) is selected from: 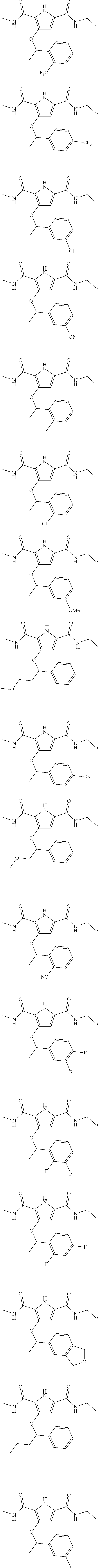 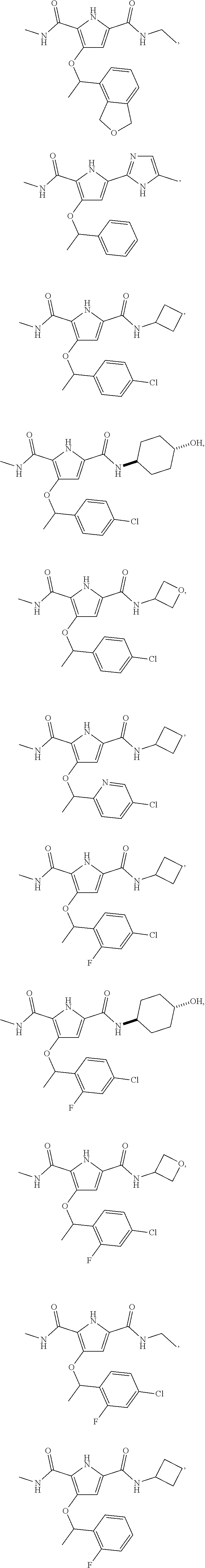 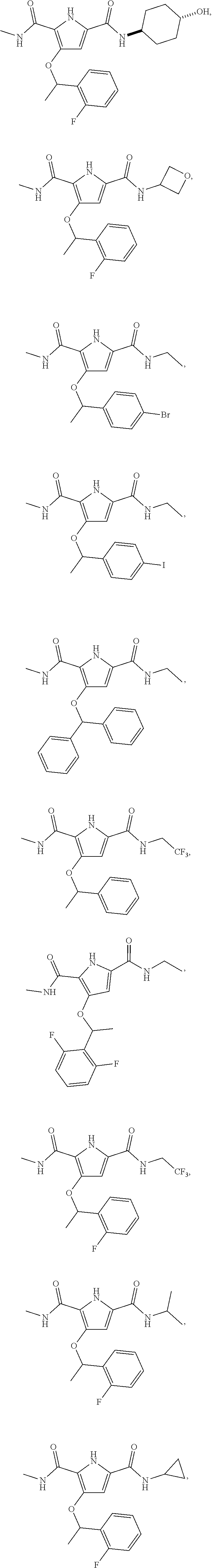 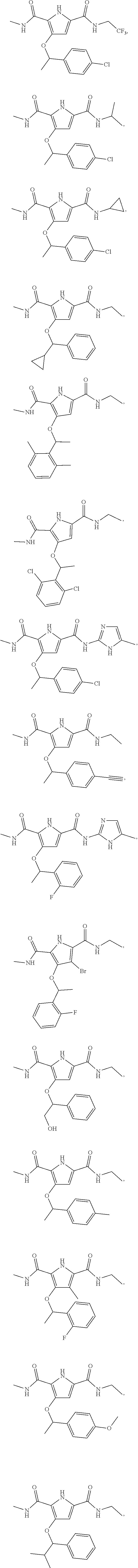 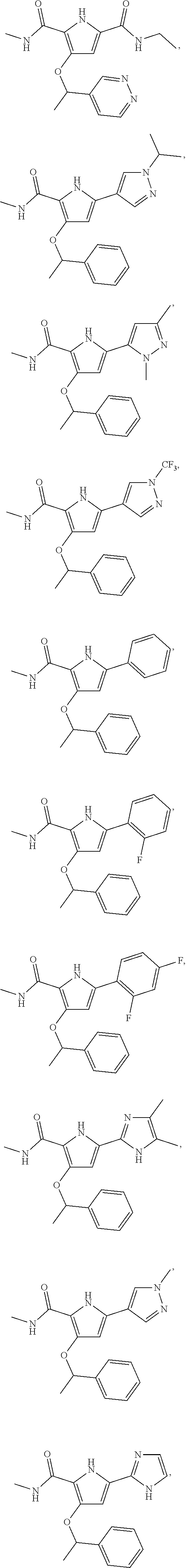 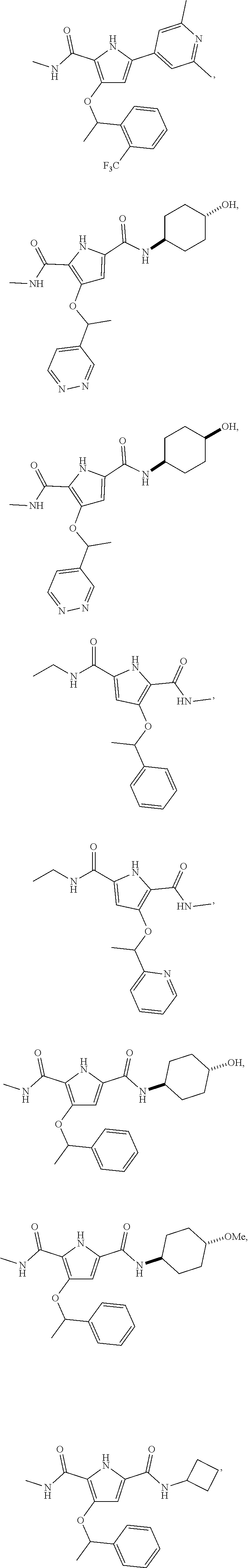 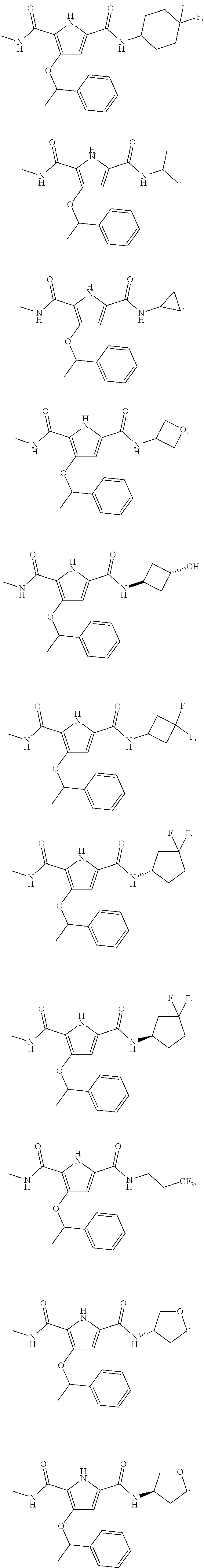 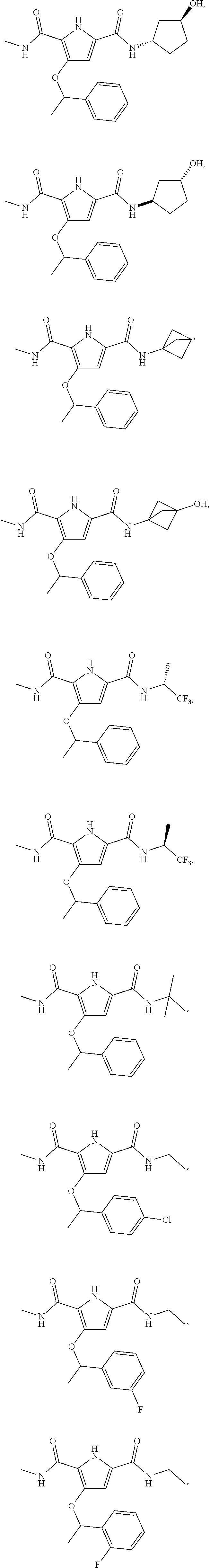 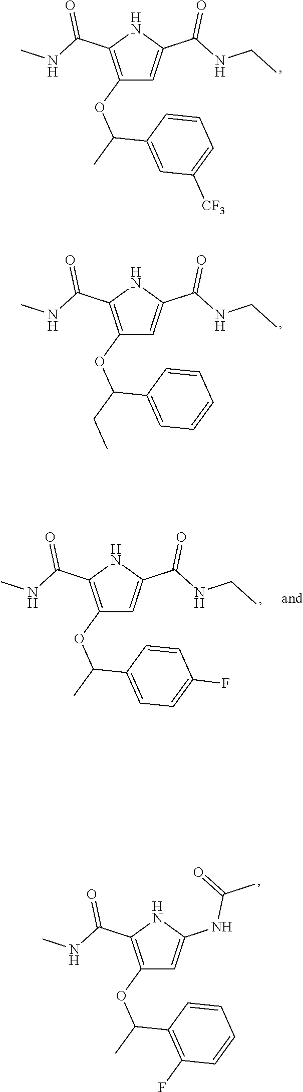
or a stereoisomer or a mixture of stereoisomers thereof.

15. The compound of claim 2, or a pharmaceutically acceptable salt or N-oxide thereof, wherein the compound according to formula (IIA) is selected from: 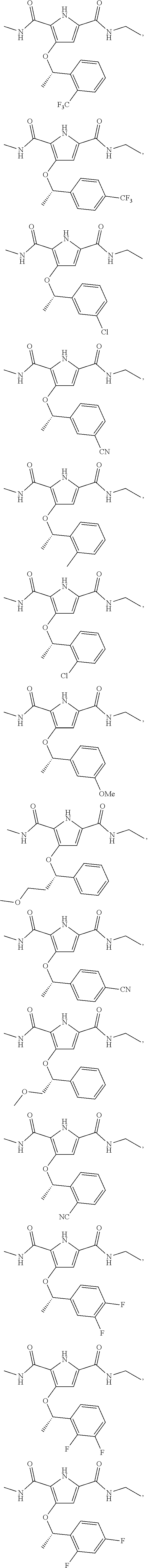 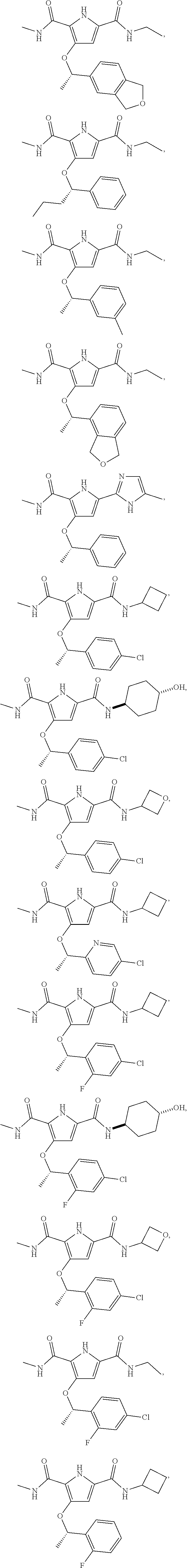 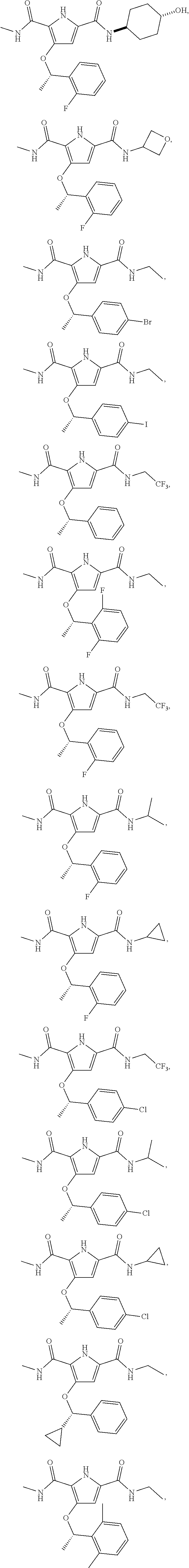 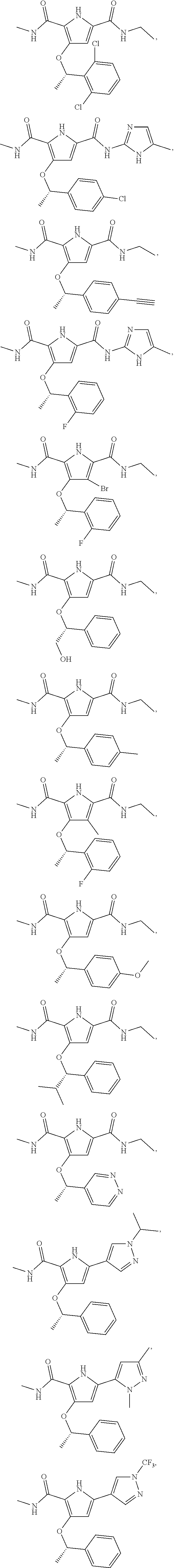 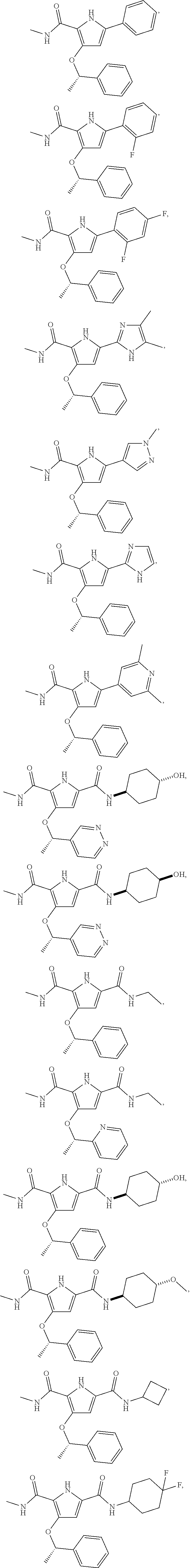 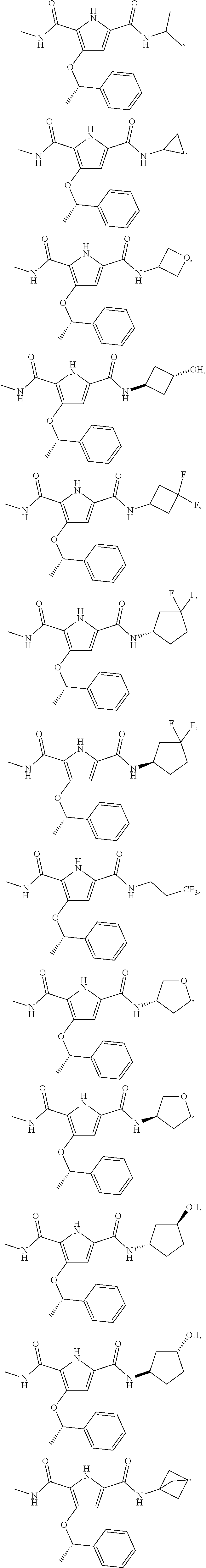 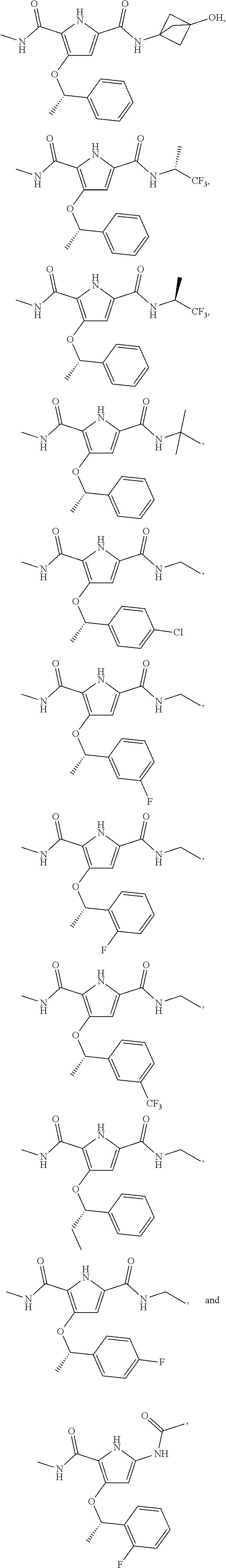
or a stereoisomer thereof.

16. A pharmaceutical composition comprising a compound of any one of claims 1 to 15, or a pharmaceutically acceptable salt or N-oxide thereof, and one or more pharmaceutically acceptable excipients.

17. A compound of any one of claims 1 to 15, or a pharmaceutically acceptable salt or N-oxide thereof, for use as a medicament.

18. A compound of any one of claims 1 to 15, or a pharmaceutically acceptable salt or N-oxide thereof, for use in treating a disease or disorder selected from an inflammatory disorder, an immune disorder, and an autoimmune disorder.

19. A compound of any one of claims 1 to 15, or a pharmaceutically acceptable salt or N-oxide thereof, for use in treating a cancer.

20. A compound of any one of claims 1 to 15, a tautomer, a stereoisomer or a mixture of stereoisomers, a pharmaceutically acceptable salt, a hydrate, a deuterated derivative, or N-oxide thereof, for use in treating a disease or disorder, wherein the disease or disorder is a joint or joint-related disease or disorder.

21. The compound for use according to claim 20, wherein the joint or joint-related disease or disorder is selected from arthritis, bursitis, Ehlers-Danlos syndrome, epicondylitis, Felty Syndrome, gouty arthritis, psoriatic arthritis, osteoarthritis, rheumatoid arthritis, Still's disease, tenosynovitis, synovitis, Sjögren's Syndrome, Lyme disease, Whipple disease, bone cancer, lupus, and other autoimmune joint disorders.

22. The compound for use according to claim 20 or 21, wherein the joint or joint-related disease or disorder comprises an arthritis.

23. The compound for use according to claim 22, wherein the arthritis comprises rheumatoid arthritis.

24. The compound for use according to any one of claims 20 to 23, wherein the disorder is an arthritis and upon administration of a therapeutically effective amount of the compound, tautomer, stereoisomer or mixture of stereoisomers, pharmaceutically acceptable salt, hydrate, deuterated derivative, or N-oxide thereof there is a therapeutic effect associated with reduction in inflammation.

25. The compound for use according to claim 24, wherein the therapeutic effect associated with a reduction in inflammation is a reduction in thickness or girth of a joint or limb.

26. The compound for use according to claim 24 or 25, wherein there is reduction in arthritic scoring or severity, and
wherein the reduction in arthritic scoring or severity is a reduction in:
(a) definite redness and swelling of an ankle/wrist or apparent redness and swelling limited to individual digits, regardless of the number of affected digits;
(b) severe redness and swelling of an ankle/wrist;
(c) redness and swelling of the entire appendage including digits; and/or
(d) maximally inflamed limb with involvement of multiple joints.

27. The compound for use according to any one of claims 24 to 26, wherein the reduction is dose dependent.

28. The compound for use according to any one of claims 24 to 27, wherein the reduction is by >about 50%, or the reduction is about 50%, or the reduction is between about 20% to about 70%.

29. The compound for use according to any one of claims 18 and 20 to 28, wherein the compound, tautomer, stereoisomer or mixture of stereoisomers, pharmaceutically acceptable salt, hydrate, deuterated derivative, or N-oxide thereof is in the form of a pharmaceutical composition which further comprises a pharmaceutically acceptable carrier.

30. The compound for use according to claim 29, wherein the compound, tautomer, stereoisomer, pharmaceutically acceptable salt, hydrate, and/or deuterated derivative thereof is formulated as a suspension or partial suspension in the composition.

31. The compound for use according to claim 30, wherein the compound, tautomer, stereoisomer or mixture of stereoisomers, pharmaceutically acceptable salt, hydrate, deuterated derivative, or N-oxide thereof is micronized.

32. The compound for use according to claim 30 or 31, wherein the compound, tautomer, stereoisomer or mixture of stereoisomers, pharmaceutically acceptable salt, hydrate, deuterated derivative, or N-oxide thereof is in the form of nanoparticles.

33. The compound for use according to any one of claims 18 and 20 to 32, wherein the compound, tautomer, stereoisomer or mixture of stereoisomers, pharmaceutically acceptable salt, hydrate, deuterated derivative, or N-oxide thereof is solubilized or partially solubilized in the composition.

34. The compound for use according to of any one of claims 18 and 20 to 33, wherein the compound, tautomer, stereoisomer or mixture of stereoisomers, pharmaceutically acceptable salt, hydrate, deuterated derivative, or N-oxide thereof or pharmaceutical composition is administered locally, topically or systemically.

35. The compound for use according to of any one of claims 18 and 20 to 34, wherein the compound, tautomer, stereoisomer or mixture of stereoisomers, pharmaceutically acceptable salt, hydrate, deuterated derivative, or N-oxide thereof or pharmaceutical composition is administered orally.

36. The compound for use according to of any one of claims 18 and 20 to 35, wherein the compound, tautomer, stereoisomer or mixture of stereoisomers, pharmaceutically acceptable salt, hydrate, deuterated derivative, or N-oxide thereof or pharmaceutical composition has activity against one or more BET domains.

37. A compound of any one of claims 1 to 15, a tautomer, a stereoisomer or a mixture of stereoisomers, a pharmaceutically acceptable salt, a hydrate, a deuterated derivative, or N-oxide thereof, for use in the treatment of a joint or joint-related disease in which a therapeutic effect associated with a reduction in inflammation is achieved.

38. A compound of any one of claims 1 to 15, a tautomer, a stereoisomer or a mixture of stereoisomers, a pharmaceutically acceptable salt, a hydrate, a deuterated derivative, or N-oxide thereof, for use in the treatment of an arthritic disease in which a therapeutic effect associated with a reduction in inflammation is achieved.

39. A compound of any one of claims 1 to 15, a tautomer, a stereoisomer or a mixture of stereoisomers, a pharmaceutically acceptable salt, a hydrate, a deuterated derivative, or N-oxide thereof, for use in the treatment of a fibrotic disease or disorder.

40. The compound for use according to claim 39, wherein the disease or disorder is renal fibrosis.

41. The compound for use according to claim 39 or 40, wherein, upon administration of a therapeutically effective amount of the compound, tautomer, stereoisomer or mixture of stereoisomers, pharmaceutically acceptable salt, hydrate, deuterated derivative, or N-oxide thereof there is a therapeutic effect associated with a reduction in fibrosis.

42. The compound for use according to claim 41, wherein the reduction in fibrosis comprises a reduction in pathology in the kidneys.

43. The compound for use according to claim 42, wherein the reduction in pathology in the kidney comprises a reduction in interstitial nephritis, collagen fiber deposition, and nephropathy.

44. The compound for use according to any one of claims 41 to 43, wherein the reduction in fibrosis comprises a reduction in inflammatory tissue biomarkers.

45. The compound for use according to claim 44, wherein the inflammatory tissue biomarkers include Col1A1, TGF-b1, MCP-1, IL-1b, IL-6, and Timp1.

46. A compound of any one of claims 1 to 15, a tautomer, a stereoisomer or a mixture of stereoisomers, a pharmaceutically acceptable salt, a hydrate, a deuterated derivative, or N-oxide thereof, for use in the treatment of a fibrotic disease in which a therapeutic effect associated with a reduction in fibrosis is achieved.

47. A compound of any one of claims 1 to 15, a tautomer, a stereoisomer or a mixture of stereoisomers, a pharmaceutically acceptable salt, a hydrate, a deuterated derivative, or N-oxide thereof, for use in the treatment of renal fibrosis in which a therapeutic effect associated with a reduction in fibrosis is achieved.

48. The compound for use of any of claims 40 to 47, wherein the progression of fibrosis severity is slowed or retarded.

49. The compound for use of any of claims 40 to 47, wherein the appearance or increase of one or more indicators of fibrosis severity is slowed or retarded.
